SEQUENCE LISTING (232 sequences)

SEQ ID NO: 1 - protein, 35213 aa, Mus musculus
MTTQAPMFTQPLQSVVVLEGSTATFEAHVSGSPVPEVSWFRDGQVISTSTLPGVQISFSDGRARLMIPAVTKANSGRYSLRATNGSGQATSTAELLVTAETAPPNFSQRLQSMTVRQGSQVRLQVRVTGIPTPVVKFYRDGAEIQSSLDFQISQEGDLYSLLIAEAYPEDSGTYSVNATNSVGRATSTAELVVQGEEVVPAKKTKTIVSTAQISETRQTRIEKKIEAHFDARSIATVEMVIDGATGQLPHKTPPRIPPKPKSRSPTPPSIAAKAQLARQQSPSPIRHSPSPVRHVRAPTPSPVRSVSPAGRISTSPIRSVKSPLLIRKTQTTTMATGPEVPPPWKQEGYVASSTEAEMRETTMTSSTQIRREERWEGRYGVQEQVTISGAAAAAASASVSSSFTAGAITTGTKEVKQETDKSAAVATVVAAVDMARVREPAISAVEQTAQRTTTTAVHIQPAQEQARKEAEKTAVTKVVVAADKAKEQELKSRTREVMVTTQEQTHISHEQIRKETEKAFVPKVVISATKAKEQETRITGEITTKQEQKRITQETIRQETEEIAASMVVVATAKSTKLEAAVGVQEETAIQQDQMHLTHEQMMKETRKTVVPKVIVATPKIKEQDLVSRSREAITTKRDQVQITQEKKRKEVETTALSTIAVATAKAKEQETVLRSREAMATRQEHIQVTHGQVGVGKKAEAVATVVAAVDQARVREPREPTHVEESHSQQTTLEYGYKEHISTTKVPEQPRRPASEPHVVPQAVKPAVIQAPSETHIKTTDQMGMHISSQVKKTTDISTERLVHVDKRPRTASPHFTVSKISVPKTEHGYEASIAGSAIATLQKELSATSSTQKITKSVKAPTVKPGETRVRAEPTPSPQFPFADMPPPDTYKSQAGIEVKKEVGVSISGSTVREEHFEVLRGREAKVTETARVPAPAEVPVTPPTLVSGLKNVTVIEGESVTLECHISGYPSPKVTWYREDYQIESSIDFQITFQGGIARLMIREAFAEDSGRFTCSAVNEAGTVSTSCYLAVQVSEEFDKETTLTEKFATEEKRFVESRDVVMTDTSITEEQAGPGEPAAPFFISKPVVQKLVEGGSVVFECQIGGNPKPHVYWKKSGVPLTTGYRYKVSYNKQTGECRLVISMTFADDAGEYTIVIRNKHGETSASASLLEEADYEALVKTQQEMLYQTQMSTFIQEPKVGEIAPGFAYSEYEKEYEKEQALIRKKMAKDTVMVRTFVEDQEFHISSFEERLIKEIEYRIIKTTLEELLEEDGEEKMAVDISESEAIESGFDIRIKNYRILEGMGVTFHCKMSGYPLPKIAWYKDGKRIRHGERYQMDFLQDGRASLRIPVVLPEDEGIYTAFASNIKGNAICSGKLYVEPAAPFSAPTYMPTPEAVSRIRSVSPRSLSRSPIRMSPAMSPARMSPARMSPARMSPARMSPGRRLEETDESQLERLYKPVFVLKPASFKCLEGQTARFDLKVVGRPMPETFWFHNGQQIVNDYTHKVVIKEDGTQSLIIVPASPSDSGEWTVVAQNRAGKSTISVTLTVEAVEHQIKPAFVEKLKNVNIKEGSRLEMKVRATGNPNPDIVWLKNSDIIVPHKYPRIRIEGTRGEAALKIDSIISQDSAWYTATAINKAGRDTTRCKVNVEVEFAEPEPERKLIIPRGTYRAKEIAAPELEPLHLRYGQEQWEEGDLYDKEKQQKPFFKKKLTSLRLKRFGPAHFECRLTPIGDPTMVVEWLHDGKPLEAANRLRMINEFGYCSLDYGAAYSRDSGVITCRATNKYGTDHTSATLIVKDEKSLVEESQLPDGKKGLQRIEELERMAHEGALTGVTTDQKEKQKPDIVLFPEPVRVLEGETARFRCRVTGYPQPKVNWYLNGQLIRKSKRFRVRYDGIHYLDIVDCKSYDTGEVKVTAENPEGVTEHKVKLEIQQREDFRSVLRRAPEPKPEFHVHEPGKLQFEVQKVDRPVDTSETKEVVKLKRAERITHEKVSEESEELRSKFKRRTEEGYYEAITAVELKSRKKDESYEELLKKTKDELLHWTKELTEEEKKALAEEGKITIPTFKPERIELSPSMEAPKIFERIQSQTVGQGSDAHFRVRVVGKPDPECEWYKNGVKIERSDRIYWYWPEDNVCELVIRDVTAEDSASIMVKAINIAGETSSHAFLLVQAKQLITFTQELQDVVAKEKDTMATFECETSEPFIKVKWYKDGIEVHAGDKYRMHSDRKVHFLSVLTIDTSDAEDYSCVLVEDENIKTTAKLIVEGAVVEFVKELQDIEVPESYSGELECIISPENIEGKWYHNDVELKSNGKYSITSRRGRQNLTVKDVTKEDQGEYSFVVDGKKTTCKLKMKPRPIAILQGLSDQKVCEGDIVQLEVKVSLENVEGVWMKDGQEVQHSDRVHIVIDKQSHMLLIEDMTKEDAGNYSFTIPALGLSTSGNVSVYSVDVITPLKDVNVIEGTKAVLECKVSVPDVTSVKWYLNDEQIKPDDRVQSIVKGTKQRLVINRTHASDEGPYKLMVGRVETSCNLSVEKIKIIRGLRDLTCTETQNVVFEVELSHSGIDVVWNFKGKEIKPSSKYKIEAHGKIYKLTVLNMMKDDEGEYAFYAGENTTSGKLTVAGGAISTPLTDQTVAESQEAVFECEVANPESEGEWLKDGKHLALSNNFRGESDGHKRRLVIAAAKLDDAGEYTYKVATSKTSAKLKVEAVKIKKTLRNLTVTETQDAVFSVELTHPDVKGVQWIKNGVVLDSNDKYEISVKGTLYSLKIKNCAMADESVYGFKLGRLGASARLHVETVKIIKKPKDVTALENATVTFEVSVSHDTVPVKWFHKNVEIKPSDKHRLVSERKVHKLMLQSISPSDAGEYTAMVGQLECKAKLFVETLHITKTMKSIEVPETKAASFECEVSHFNVPSMWLKNGVEIEMSEKFKIVVQGKLHQLIIMNTSTEDSAEYTFVCGNDQVSATLTVTPIMITSMLKDINAEEKDTITFEVTVNYEGISYKWLKNGVEIKSTDRCQMRTKKLTHSLNIRNVHFGDAADYTFVAGKATSTATLYVEARHIEFRKHIKDIKVLEKKRAMFECEVSEPDITVQWMKDGQELQIADRIKIQKEKYVHRLLIPSTRMSDAGKYTVVAGGNMSTANLFVEGRDVRIRSIKKEVQVIEKQRAVVEFEVNEDDVDAHWYKDGIEINFQVQERHQYVVERRIHRMFISETRHSDAGEYTFVAGRNRSSVTLYVNAPEPPQVLQELQPVTVQSGKPARFCAVISGRPQPKISWYKEEQLLSTGFKCKFLHDGQEYTLLLIEAFPEDAAVYTCEAKNDYGVATTSASLSVEVPEVVSPDQEMPVYPPAIVTPLQDTVTSEGRPARFQCQVSGTDLKVSWYCKDKKIKPSRFFRMTQFEDTYQLEIAEAYPEDEGTYAFVANNAVGQVSSTATLRLEAPESILHERIGQQIEMEMKEIASLLSAEEDFQTYSSDLRLPNANETLELLSEPPARSTQFDSRQEGAAPVFIREISDVEISVEDVAKLSVTVTGCPKPKIQWFFNGMLLTPSADYKFVFDGDTHSLIILFTRFQDEGEYTCLASNEYGKAVCSAHLRISPRGERSTEMESGEKKALEKPKGPCPPYFFKELKPVHCGPGIPAVFEYSVHGEPAPTVLWFKEDMPLYTSVCYTIIHSPDGSGTFIVNDPQRGDSGLYLCKAQNLWGESTCAAELLVLPEDTDVPDASCKEESTLGVPGDFLETSARGPLVQGVDSRQEITAFAEGTISKAALIAEETLQLSYERSVDDSEVGTGVTIGAQKLPPVVLSTPQGTGELPSIDGAVHTQPGRGPPPTLNLQAVQAQTTLPKEATLQFEEPEGVFPGASSAAQVSPVTIKPLITLTAEPKGNYPQSSTAAPDHALLSSVAAETLQLGEKKIPEVDKAQRALLLSQSLAEGCVESLEVPDVAVSNMRSEPQVPFQHTCTEGKILMASADTLKSTGQDVALRTEEGKSLSFPLALEEKQVLLKEEQSEVVAVPTSQTSKSEKEPEAIKGVKEVREQELLSKETLFPSMPEEQRLHLKTQVRRALQAAVAREQANLFSEWLRNIDKVEVTAVNFTQEPKRILCTYLITSVSSLTEELTVTIEDIDPQMANLETGLKDALCSIVCEERNILMAEDPRIHEEDKIDVQGGRDHLSDAQKVETVIEAEADSKYLVSKEEVSWSKVESQLKDGDTNEVPQAETLKLAEESGTQKTSTEMSQEEAEGTLADLCPAVLKHLVDTISEEGDTVHLTSSISNAKEVHWYFKGNLVPSDGKFKCLKEQNAYTLVIEAVKTEDEGEYVCEASNDSGKAKTSAKLTVGERAAPVIKRRIEPLEVALGHLAKFTCEIQGAPNVRFQWFKAGREIYESDKCSIRSSNYVSSLEILRTQVVDCGEYTCKASNEYGSVSCTATLTVTEAYPPTFLSRPKALTTFVGKAAKFLCTVSGTPVIEIIWQKDGAALSPSPDCRVTDADNKHSLELSNLTVQDRGIYSCKASNKFGADICQAELTIIDKPHFIKELEAVQSAINKKIHLECQVDEDRKVTITWSKDGQKLPAGKDYKIYFEDKIASLEIPLAKLKDSGTYTCTASNEAGSSSSSAAVAVREPPSFVKKVDPSYLMLPGESARLHCKLKGSPVIQVTWFKNNKELSESNTVRMSFVNSEAILDITDVKVDDSGTYSCEATNDVGSDSCSTEVVIKEPPSFIKTLEPADIVRGANALLQCEIAGTGPFEVNWFKDKKQIRSSKKYRLFTQKTFVYLEISSFNSADVGDYECVVANEVGKCGCVATHLLKEPPTFVKKVDDFTALAGQTVTLQAAVRGSEPISVMWMKGQEVIKEDGKIKMSFSNGVAVLTIPDVQISLGGKYTCLAENEAGSQTSVGELIVKEPAKIIERAELIQVTAGDPATLEYTVSGTPELKPKWYKDGRPLVASKKYRISFKNNIAQLKFYSAELHDSGQYTFEISNEVGSSSCETTFTVLDRDIAPLFTKPLRNVDSVVGGACRLDCKIAGSLPMRVSWFKDGKELTASDRYQIAFVEGTASLEISRVDMNDAGNFTCRATNSVGSKDSSGALIVQEPPSFVTKPGSRDVLPGSAVCLKSAFQGSAPLTIKWFKGDKELVSGGSCYITKETSESSLELYAVKTSDSGTYTCKVSNVAGSVECSADLFVKEPATFIEKLEPSQLLKKGDGTQLACKVTGTPPIKITWFANDRELRESSKHKMSFAESTAVLRLTDVAIEDSGEYMCEAQNEAGSDHCTGIVIVKESPYFTKEFKSIEVLKEYDVMLLAEVAGTPPFEITWFKDNTTLRSGRKYKTFLQDQLVSLQVLKFVAADAGEYQCRVTNEVGSSTCSARVTLREPPSFIKKIEATSSLRGGTAAFQATLKGSLPITVTWLKDNDEITEDDNIRMTFENNVASLYLSGIEVKHDGKYVCQAKNDAGIQRCSALLSVKEPATIMEEAVSIDVTQGDPATLQVKFSGTKEISAKWFKDGQELTLGPKYKISVTDTVSILKIISTEKKDSGEYTFEVQNDVGRSSCKASINVLDLIIPPSFTKKLRKMDSIKGSFIDLECIVAGSHPISIQWFKDDQEISASDKHKFSFHDNTAFLEISQLEGTDSGTYTCSATNKAGHSQCSGHLTVKEPPYFVEKPQSQDVNPGTRVQLKALVGGTAPMTIKWFKDNKELHPGAARSVWKDDTSTILELFSAKAADSGTYICQLSNDVGTTSSKATIFVKEPPQFIKKPSPVLVLRNGQSTTFECQVTGTPEIRVSWYLDGNEITDLRKYGISFVDGLATFQISNARVENSGTYVCEARNDAGTASCSIELKVKEPPIFIRELEPVEVVKDSDVELECEVMGTTPFEVTWLKNNKEIRSGKKYTMSEKMSVFYLHITKCDPSDVGEYQCIIANEGGSCACSARVALKEPPSFIKKIENVTTVLKSSATFQSTVAGSPPISITWLKDDQILEENDNVHISFEDSVATLQVRSVDNGHSGRYTCQAKNESGIERCYAFLLVQEPAQIIEKAKSVDVTEKDPVTLECVVAGTPELKVKWLKDGKQIVPSRYFSMSFENNVASFRIQSVMKQDSGQYTFKVENDFGSSSCDAYLRVLDQDIPPSFTKKLTKMDKVLGSSIHMECKVSGSLPISAQWFKDGKEISTSAKYRLVCHENTVSLEVSNLELEDTANYTCKVSNVAGDNACSGILTVKEPPSFLVKPERQQAIPDSTVEFKAVLKGTPPFKIKWLKDDVELVSGPKCFIGLEGSTSFLNLYSVDSSKTGQYTCQVTNDVGSDSCTTMLLVTEPPKFVKKLEASKIIKAGDSARLECKITGSPEIQVVWYRNEHELTASDKYQMTFIDSVAVIQMNSLGTEDSGDFICEAQNPAGSTSCSTKVIVKEPPVFSSFPPIVETLKNTEVSLECELSGTPPFEVVWYKDKRQLRSSKKYKVASKNFHASIHILNVESTDIGEYHCKAQNEVGSDACVCAVKLKEPPKFISKLNSLTVVAGEPAELQASIEGAQPISVQWLKEKEEVIRESENIRISFVNNVATLQFAKVEPANAGKYICQVKNDGGVRENMATLTVLEPAVIIEKAGSMTVTVGETCALECKVAGTPELSVEWYKDGKLLTSSQKHKFSFYNKISSLKILSVEKEDAGTYTFQVQNNVGKSSCTAVVDVSDRMVPPSFTRRLKDTGGVLGTSCILECKVAGSSPISIAWFHEKTKIVSGAKYQTTFSDNVCTLQLNSLDSSDMGSYTCVAANVAGSDECRALLTVQEPPSFVKEPEPLEVLPGKNITFTSVIRGTPPFKVGWFRGARELVKGNRCNIYFEDTVAELELFNIDISQSGEYTCVVSNNAGQASCTTRLFVKEPATFVKKLSDHSVEPGKSIILEGTYTGTLPISVTWKKDGVSITPSERCNIVTTEKTCILEILSSTKGDAGHYSCEIENEAGRDACDALVSTLEPPYFVTELEPLEASVGDSVSLQCQVAGTPEITVSWFKGDTKLRSTPEYRTYFTNNVATLVFNKVSINDSGEYTCMAENSIGTAASKTIFRIQERQLPPSFARQLKDIEQTVGLPVTLTCRLNGSAPIQVCWYRDGVLLRDDENLQMSFVDNVATLKILQTDLSHSGQYSCSASNPLGTASSTARLTAREPKKSPFFDIKPVSIDVIAGESADFECHVTGAQPMRVTWSKDNKEIRPGGNYTITCVGNTPHLRILKVGKGDSGQYTCQATNDVGKDMCSAQLSVKEPPKFIKKLDASKVAKQGESIQLECKISGSPEIKVVWFRNDSELHESWKYNMSFVNSVALLTINEASAEDTGDYICEAHNGVGDASCSTALKVKAPPVFTQKPPPVGALKGSDVILQCEISGTPPFEVVWVKDRKQVRSSKKFKITSKNFDTSLHIFNLEAPDIGEYHCKATNEVGSDTCACTVKFKEPPRFVKKLSDASTLIGDPVELQAVVEGFQPISVVWLKDKGEVIRESENVRISFVDNIATLQLGSPEASQSGKYVCQIKNDAGMRECSAVLTVLEPATIVEKPEPMTVTTGNPFTLECVVAGTPELSAKWFKDGRELSSGSRHHITFVRNLASLKIPSAEMNDKGLYTFEVENRVGKSSCTVSVHVSDRVVPPSFVRRLKDTSATLGASVVLECRVSGSAPISVGWFLDGNEIISSPKCQSSFADNVCTLTLSSLEPSDTGAYTCVAANVAGQDESSAVLTVQEPPSFEQTPDSVEVLPGMSLTFTSVIRGTPPFKVKWFKGSRELVSGEACTISLEDFVTELELLEVEPGQSGDYSCLVTNDAGSASCTTHLFVKEPATFVKRLADTSVETGSPIVLEATYSGTPPISVSWMKNEYPLSQSPNCGITTTEKSSILEILESTIEDYAQYACLIENEAGQDICEALVSVLEPPYFIEPLEHVEAAIGEPITLQCKVDGTPEIRISWYKEHTKLRSAPAYKMQFKNNVASLVINKVDHSDVGEYTCKAENSVGAVASSAVLVIKERKLPPSFARKLKDVHETLGFPVAFECRINGSEPLQVSWYKDGELLKDDANLQMSFVHHVATLQILQTDQSHVGQYNCSASNPLGTASSSAKLILSEHEVPPFFDLKPVSVDLALGESGSFKCHVTGTAPIKITWAKDNREIRPGGNYKMTLVENTATLTVLKVAKGDAGQYTCYASNVAGKDSCSAQLGVQEPPRFIKKLDQSRIVKQDEYTRYECKIGGSPEIKVLWYKDEVEIQESSKFRMSFEDSVAILEMHSLSVEDSGDYTCEARNAAGSASSSTSLKVKEPPVFRKKPFPVETLKGADVHLECELQGTPPFQVSWHKDKRELRSGKKYKIMSENLLTSIHILNVDTADIGEYQCKATNDVGSDTCVGSVTMKAPPQFVKKLTDISTIIGKEVQLQTTIEGAEPISVAWFKDKGEIVRESDNIWISYSENIATLQFSRAEPANAGKYTCQIKNDAGMQECYATLSVLEPAAIVEKPESIKVTTGDTCTLECTVSGTPELSTKWFKDGKELTSDNKYKISFFNKVSGLKIINVVPGDSGVYSFEVQNPVGKDSCKVSIQVSDRIIPPSFTRKLKETNGLSGSSVVMECKVYGSPPISVLWFHDGNEISSGRKYQTTLTDNTCALTVNMLEEADAGDYTCIATNVAGSDECSAPLTVREPPSFVQKPDPMDVLTGSNVTFTSIVKGSPPFTVSWFKGSTELVPGARCNVSLQDSVGELELFDVDTSQSGEYTCIVSNEAGRASCTTRLFVKAPAIFVKRLNDYSIEKGKPLILEGTFSGTPPISVTWKKNGINVIASQRCNITTTEKSAILEILSSTVEDSGQYNCYIENASGKDSCSAQILILEPPYFVKQLEPVKVTVGDSASLQCQLAGTPEIGVSWYKGDTKLRPTATCKMHFKNNVATLVFTQVDSSDSGEYICRAENSVGEVSSSTFLTVQEQKLPPSFSRQLRDVQETVGLPVVFECAVSGSEPISVSWYKDGKPLKDSPNIQTSFLDNIATLNIFKTDRSLSGQYSCTATNPIGSASSGAKLILTEGKNPPFFDIPLAPMDAVVGESADLECHVTGTQPIKVTWAKDNREIRSGGNYQISYLENSAHLTIVKVDKGDSGQYTCYAVNEVGKDSCTAQLNIKERLIPPSFTKKLSETVEETEGNSFKLEGRVAGSQPITVAWYKNNVEIHPTSNCEIMFKNNALLLQVKRASMADAGLYTCKATNDAGSALCTSSIVIKEPKKPPVFDQHLAPVTASEGDSVQLSCHVQGSEPIRIQWLKAGREVKPSDRCSFSFASGTAMLELKETAKADSGDYVCKASNVAGSDTSKCKVTIKEKPAAAPAAKKAAVDGKLFFVSEPQSIRVVEKTTATFIAKVGGDPIPNVKWTKGKWRQLNQGGRILIHQKGDEAKLEIRDTTKTDSGLYRCVAFNKHGEIESNVNLQVDERKKQEKIEGDLRAMLKKTPALKKGSGEEEEIDIMELLKNVDPKEYEKYARMYGITDFRGLLQAFELLKQSQEEETHRLEIEELEKSERDEKEFEELVAFIQQRLTQTEPVTLIKDIENQTVLKDNDAIFEIDIKINYPEIKLSWYKGTEKLEPSNKYEISIDGDRHTLRVKNCQPKDQGNYRLVCGPHIASAKLTVIEPAWERHLQDVTLKEGQTCTMTCQFSVPNVKSEWFRNGRVLKPQGRVKTEVEHKVHKLTIADVRAEDQGQYTCKHEDLETSAELRIEAEPIQFTKRIQNIVVSEHQSATFECEVSFDDAIVTWYKGPTELTESQKYNFRNDGRCHYMTIHNVTPDDEGVYSVIARLEPRGEARSTAELYLTTKEIKLEMKPPDIPDSRVPIPTMPIRAVPPEEIPPAVAPSIPLLLPLPEEKKPPAKRIEVTKKGVKKDTKKVVTKPKEEAPPPPVAKKPPPPTPMIPAKASEIIDVSSKAEEVKITTITRKKEVHKEKEAVYEREEAVYEKKVHIEPWEEPYEELETEPYTEPYEEPYYEEPDEDYEEIKVEAKKQVHEEWEEDFEEGQEYYEREEGYDEGEEEWEEIYHEREIIQVQKEVHEELHEKKIPAKVPEKKVPPPKVVKKPVVEKVEKTTRRMEEEKVQVIKVPEVSKKIVPQKPSRTPVQEEIIEVKVPAVHTKKMVISEEKMFFASHTEEEVSVSVPEVQKKTVTEEKIHVAVSKKIEPPPKVPEPPKKPVPEEVVPVPIPKKVEPPAAKVPEAPKKPVPEEKKPVPIPKKKEPAAPPQVPEAPKKPAPEEKIPVPVTKKKEAPPAKVPEVQKKVVTEEKIAIITQREESPPPAVPEIPKKKVPEEKRPVPRKEEVPPPKVPVPPKKAVPEAVVPAPIPKKAPPRAEVSKKTVVEEKRFAAEEKLSVAVPQRVELMRHEEEEWTYSEEEERVSVSVYREEERDEEEAEITEYEVLEEPEEYVVEEKMHFISKKVEVEPAKVPEKKIIPKPKVPAKIEEPPPTKVPEPPKKIVPEKKVPAPAPKKVPPAKAPEESKRPVPEKRAPAEEVGIEEPPPTKVAERHMKITQEEKVLVAVTKKEAPPRARVPEEPKKVAPEERFPKLKPRREEEPPAKVTEVRKRAVKEEKVSIEVPKREPRPTKEVTVTEEKKWSYTREEETVSEHREEEYEDYEDYEEYKEFEEYEPTEEYDQYDEYAEREVEHYEEHEEYVTEPKKPVPVKPAQEPVPAKPKAPPPKVLKKAVPEEKAPLPIQKKLKPLPPKAPEEPKKVVEEKIQISITKREKQQVTEPVAKVPMKPKRVVPEAKIPAPTKEVAVPVRVPGVPKKRELEEVVVFKEEVEAHEEYIVEEEEEYVHEEEYVHKEEYVHEEEYVHKEEYIHEEEEHLHEEEETIAEEEVVPVAPVKVPVVPKKPVPEEKKPVPVPKKKEAPPAKVPEIPKKPEEKVPVPIPKKEKAPPAKVPEVPKKPVPEEKPPVPVPKKVEPPPAKVPEVPKKPVPEKKVPAPTPKKVEAPPAKVPEVPKKPIPEEKKPTALLKKMEAPPPKAPKKREVVPVPVALPREEEEEEVPFEEVPEEEILPEEEVPSEEEAPPEEVPPEEEEVLPEEEEVLPEEEEVLPEEEEVQPEEEALPEIKPKVPKPAPVPKKTVPEKKVPVPVPKKVEPPPPPKVPEIKKKVPEKKVVVPKKEEAPPTKVPEVSKKVEERRIIPPKEEEVPPAEVYEEAEEPTPEEIPEEPPSIEEEEIVEEEEEEEEVLPPRAPEVVKKAVPEAPTPVPKKAEAPPAKVPKKIPEEKVPVPVQKKEAPPAKVPEKKKIPEKKVPVPKKEAVPPAKGKAVFEEKISVAYQQEELVQERIELELVEAKVEEAFEEEEFHEVQEYFEEEEFHEVEEFIRVEERRFQEEHKVEEVHRVIEFLEAEEVEVYEKPKIPPKKGPEVSEKVIPPKKPPTKVIPRKEPPAKVPEVTKKTVVEEKIRAPEEPKVPAPKAPEVPKKITPEEKVREAVPKKPEVPPPKVPEVPKKIIQEEKLPVVLPEDTEIYMYEASEETVIEEEHVTLPQKARLKVAKVPAPPQTVVTEEKTYVTIRKTRETLALKESETTREAFPELKSYKAVPEIPEPPSPEDLEIIEDVLPEKRPPASKRRKTQLPTAPEAPREMPPEMNTFEEISVEPEMLPTQVLDTYQEATVEKKTLRISRKKPELPSDEEVPEAPREVVAKKKVLPPQVPEVVPVKVPGAPKEVVSERKSLEEPPKKPAVRPVTVPEEPKEVIPEKKVSLVPPKKPAAPPVTVPEAPEEVFSEDEETLAPPQEPEAPPAKVPEAPKEVVPEKKVSVVPPKKPEAPPAKVPEAPKEAAPEKKVPVAPKKKPEAPPVKVPEAPKKVVPEKKLPVAAPKKPEAPAAEVPEVPKTAVPQKKIPEAIPPKPESPPLEVPEVPPKEVTPEKKVPAAPPTKPEIPPPKVPEAPQAAVVEEKTPEALPKKAEAAPVPVPQVQETVPEKTRPVGPPKKPEATTVPVPKVQKTIPEKTRPEAPPKRPEARTVPETVPEKTRPMAPPKKPEATTLPVPEVQETVPEKTRPVGPPKKPEATTVPVPEVQETIPEKTRPPKKPEATPVPVPEVQETVPEKTRPVGPPKKPEATTVSVPEVQETIPEKTRPAAPPKKPEATAVPETIPEKTRPEAPPKRPEATTVPVPEADQAVVPEKKVPRVPPKKVEAPPITVPEEPKEVIPEKKVSLVPPKKPAAPPVTVPEAPEEVFSEDEETLAPPQEPEAPPAKVPEAPKEVVPEKKVSVVPPKKPEAPPAKVPEAPKEAAPEKKVPVAPKKKPEAPPVKVPEAPKKVVPEKKLPVAAPKKPEAPAAEVPEVPKAAVPQKKIPEAIPPKPESPPPEVYEEPEEEIVPEEPPEEAVEEPVPAPPPKVTEPPKKPVPEKKAPPAVVKKPEPPPAKVPEVPKEAPPEKKVPPKKPEAPPAKVPEVPKEVVTEKKVAVPKKPEVPPAKVPEVPKKPVIEEKPAIPVVEKVASPPAEVYEEPEEVTAEEEEPAPAVEEEEYEAPPPPAPVPEEPKKVVPEKKFPVIKKPEAPPPKVPEVPKKAVPVKKVPVVKKPEPPEAEVPEVPKKLVPVKKEPVPVTKKTEVLPEKVPEAPKKITPEKKESVPVPEEPEAPPASVEETPEETIYEEKATITIGRKETPPVEEREIEKFIQPEEPELEPEPEEIPVQEPEPEKKVIEKPKLKPRPPARPPSPPKEDVKEKMFQLKAVSKKKVPEKPEVVEKVEPAPLKVPTAEKKVRKLLPEPKPQPKEEVVLKSVLRKKPEEEEPKVEPKKVEKAKKPEEPQPPPKAVEVEAPPEPTPKERKVPEPAKVPEIKPAIPLPGPEPKPKPEPEVKTMKAPPIEPAPTPIAAPVTAPVVGKKAEAKPKDEAAKPKGPIKGVAKKTPSPIEAERKKLRPGSGGEKPPDEAPFTYQLKAVPLKFVKEIKDIVLTEAESVGSSAIFECLVSPSTAITTWMKDGSNIRESPKHRFIADGKDRKLHIIDVQLSDAGEYTCVLRLGNKEKTSTAKLIVEELPVRFVKTLEEEVTVVKGQPLYLSCELNKERDVVWRKDGKIVVEKPGRIVPGVIGLMRALTINDADDTDAGTYTVTVENANNLECSSCVKVVEIIREWLVKPIRDQHVKPKGTAVFTCDIAKDTPNIKWFKGYDEIPLEPNDKTEILKEGNHLFLKVKNAMPEDIDEYAVEIEGKRYPAKLTLGEREVELLKPIEDVTIYEKESASFDAEISEEDIPGEWKLKGELLRPSPTCEIKAEGGKRFLTLHKVKLDQAGEVLYQACNAITTAILTVKEIELDFAVPLKDVTVPEKRQARFECVLTREANVIWSKGPDIIKASDKFDIIADGKKHILVINDSQFDDEGVYTAEVEGKKTSAQLFVTGIRLKFISPLEDQTVKEGQTATFVCELSHEKMHVVWFKNDVKLHTTRTVLMSSEGKTYKLEIRETTLDDISQIKAQVKNLSSTANLKVLEADPYFTVKLHDKTGVEKDEIILKCEVSKDVPVKWFKDGEEIVPSPKHSVKTDGLRRILKIKKAELKDKGEYVCDCGTDTTKANVTVEARLIKVEKPLYGVEVFVGETARFEIELSEPDVHGQWKLKGEPLTASPDCEIIEDGKKHVLVLYNCQLDMTGEISFQAANAKSAANLKVKELPLIFITPLSDVKVFEKDEAKFECEVSREPKTFRWLKGTQEITGDDRFELIKDGTRHSLVIKSAAFEDEAKYMFEAEDKRTSGKLIIEGIRLKFLTPLKDVTAKERENAVFTVELSHDNIPVSWFKNDQRLHTSKRVSMHDEGKTHSITFKDLSIDDTSQIRVEAMGISSEAKLTVLEGDPYFTGKLQDYTGVEKDEVILQCEISKADAPVKWFKDGKEIKPSKNVVIKADGKKRMLILKKALKSDIGQYTCDCGTDQTSGKLDIEDREIKLVRPLYSVEVMETETARFETEISEDDIHANWKLKGEALLQTPECEIKEEGKIHVLILHNCRLDQTGGVDFQAANVKSSAHLRVKPRVIGLLRPLKDVTVTAGETATFDCELSYEDIPVEWYLKGKKLEPNDKVVTRSEGRVHTLTLRDVKLEDAGEVQLTAKDFKTQANLFVKEPPVEFTKPLEDQTVEEEATAVLECEVSRENAKVKWFKNGTEILKSKKYEIVADGRVRKLIIHGCTPEDIKTYTCDAKDFKTSCNLNVVPPHVEFLRPLTDLQVKEKETARFECEISKENEKVQWFKDGAEIKKGKKYDIISKGAVRILVINKCLLNDEAEYSCEVRTARTSGMLTVLEEEAVFTKNLANLEVSEGDTIKLVCEVSKPGAEVIWYKGDEEIIETGRFEILTDGRKRILIIQNAQLEDAGSYNCRLPSSRTDSKVKVHELAAEFISKPQNLEILEGEKAEFVCTISKESFEVQWKRDDQTLESGDKYDIIADGKKRVLVVKDATLQDMGTYVVMVGAARAAAHLTVIEKLRIIVPLKDTKVKEQQEVVFNCEVNTEGAKAKWFRNEEAIFDSSKYIILQKDLVYTLRIRDARLDDQANFNVSLTNHRGENVKSAANLIVEEEDLRIVEPLKDIETMEKKSVTFWCKVNRLNVTLKWTKNGEEVAFDNRISYRIDKYKHSLIIKDCGFPDEGEYVVTAGQDKSVAELLIIEAPTEFVEHLEDQTVTEFDDAVFSCQLSREKANVKWYRNGREIKEGKKYKFEKDGSIHRLIIKDCRLEDECEYACGVEDRKSRARLFVEEIPVEIIRPPQDILEAPGADVIFLAELNKDKVEVQWLRNNMIVVQGDKHQMMSEGKIHRLQICDIKPRDQGEYRFIAKDKEARAKLELAAAPKIKTADQDLVVDAGQPLTMVVPYDAYPKAEAEWFKENEPLSTKTVDTTAEQTSFRISEAKKDDKGRYKIVLQNKHGKAEGFINLQVIDVPGPVRNLEVTETFDGEVSLAWEEPLTDGGSKIIGYVVERRDIKRKTWVLVTDRADSCEFTVTGLQKGGVEYLFRVSARNRVGTGEPVETDSPVEARSKYDVPGPPLNVTITDVNRFGVSLTWEPPEYDGGAEITNYVIELRDKTSIRWDTAMTVRAEDLSATVTDVVEGQEYSFRVRAQNRIGVGKPSAATPFVKVADPIERPSPPVNLNASEQTQSSVQLTWEPPLKDGGSPILGYIIERREEGKDNWIRCNMKPVPELTYKVTGLQKGNKYLYRVSAENAAGVSDPSEILGPLTADDAFVEPTMDLSAFKDGLEVIVPNPIKILVPSTGYPRPKATWTFGDQVLEEGDRVKMKTISAYAELVISPSERTDKGIYTLTLENPVKSISGEINVNVIAPPSAPKELKFSDITKDSVHLTWEPPDDDGGSPLTGYVVEKRDMSRKTWTKVMDFVTDLEFTVPDLVQGKEYLFKVCARNKCGPGEPAYTDEPVNMSAPATVPDPPENVKWRDRTANSIFLTWDPPKNDGGSRIKGYIVEKCPRGSDKWVACGEPVPDTKMEVTGLEEGKWYAYRVKALNRQGASKPSKPTEEIQAVDTQEAPEIFLDVKLLAGITVKAGTKIELPATVTGKPEPKITWTKADTLLKPDQRITIENVPKKSTVTITDSKRSDTGTYIIEAVNVCGRATAVVEVNVLDKPGPPAAFDITDVTNESCLLTWNPPRDDGGSKITNYVVERKATDSDVWHKLSSTVKDTNFKATKLTPNKEYIFRVAAENMYGVGEPVQAAPIIAKYQFDPPGPPTRLEPSDITKDAVTLTWCEPDDDGGSPITGYWVERLDPDTDKWVRCNKMPVKDTTYRVKGLTNKKKYRFRVLAENLAGPGKPSRSTEPILIKDPIDPPWPPGKPTVKDIGKTSLVLNWTKPEHDGGAKIESYVIEMLKTGTDEWVRVAEGVPTTEHLLTGLMEGQEYSFRVRAVNKAGESEPSEPSDPVLCREKLYPPSPPRWLEVINITKNTADLKWTVPEKDGGSPITNYIVEKRDVRRKGWQTVDTTVKDTKCTVTPLTEGSLYVFRVAAENAIGQSDYTEIGDSVLAKDTFTTPGPPYALTVVDVTKRHVDLKWEPPKNDGGRPIQRYIIEKKEKLGTRWVKAGKTSGPDCNFRVTDVIEGTEVQFQVRAENEAGVGHPSEPTEILSIEDPTSPPSPPLDLHVTDAGRKHIAIAWKPPEKNGGSPIIGYHVEMCPVGTEKWMRVNSRPIKDLKFKVEEGIVPDKEYVLRVRAVNAVGVSEPSEISENVVAKDPDCKPTIDLETHDIVVIEGEKLNIPVPFRAVPVPTVSWHKDGKEVKASDRLTMKNDHISAHLEVPKSVHADAGVYTITLENKLGSATASINVKVIGLPGPCKDIKASDITKSSCKLTWEPPEFDGGSPILHYVLERREAGRRTYIPVMSGENKLSWTVKDLIPNGEYFFRVKAVNKIGGGEYIELKNPVIAQDPKQPPDPPVDVEVHNPTAKAMTITWKPPLYDGGSKIMGYIIEKITKGEDRWKRCNEHLVPVLTYTAKGLEEGKEYQFRVRAENAAGIGEPSRATPPTKAVDPIDAPKVILRTSLEVKRGDEIALDATISGSPYPTITWIKDENVIVPEEIKKRAAPPVRRKKGEAEEEEPFSLPLTERLSINNSKQGESQLRIRDSLRPDHGQYMIKVENDHGVAKAPCSVSVLDTPGPPINFVFEDIRKDSVLCKWEPPLDDGGSEIINYTLEKKDKTKPDSDWIVITSTLRNCKYSVTKLIEGKEYLFRVRAENRFGPGPPCVSKPLLAKDPFEPPDAPDKPIVEDVTSNSMLVKWNEPKDNGSPILGYWLEKREVNSTHWSRVNKTLLSSLKTKVDGLLEGLTYVFRVCAENAAGPGKFSPPSDPKTARDPISPPGPPVPRVADTSSTTIELEWEPPAFNGGGEIMGYFVDKQLVGTNEWSRCTEKMIKVRQFTVKEIREGADYKLRVSAVNAAGEGPPGETEPVTVAEPQEPPTVELDVSVKGGIQIMAGKTLRIPAEVTGRPVPTKVWTIEEGELDKERVIIENVGTKSELIIKNALRKDHGRYVITATNSCGSKFAAVRVEVFDVPGPVLDLKPVVTNRKMCLLNWSDPADDGGSDITGFIIERKDAKMHTWRQPIETERSKCDITGLIEGQEYKFRVIAKNKFGCGPPVEIGPILAVDPLGPPTSPERLTYTERTKSTITLDWKEPRSDGGSPIQGYIIEKRRHDKPDFERVNKRLCPTTSFLVENLDEHQMYEFRVKAVNDVGESEPSLPLNVVIQDDEVPPTIKLRLAVRGDTIKVKAGEPVNIPADVTGLPMPKIEWSKNEKVIDKPTDTLNITKEEVSRSEAKTELSIPKAAREDKGTYTITASNRLGSVFRNVHVEVYDRPSPPRNLAVTDIKAESCYLTWDAPLDNGGSEITHYIIDKRDASRKKSEWEEVTNTAVERRYGIWKLIPNGQYEFRVRAVNKYGISDECKSDKVVIQDPYRLPGPPGKPKVLERTKGSMLVSWTPPLDNGGSPITGYWLEKREEGGTYWSRVSRAPITKVGLKGVEFNVPRLIEGVKYQFRAMAINAAGIGPPSEPSDPAVAGDPIYPPGPPSCPEVKDKTKSSISLAWKPPAKDGGSPIKGYIVEMQEEGTTDWKPVNEPDKLLTACECVVPNLKELRKYRFRVKAVNEAGESEPSDTTGEIPATDIQEVPEVFIDIGAQDCLVCKAGSQVKIPAVIKGRPTPKSSWEFDGKAKKAMKDGVHDIPEDAQLETAENSSVIIIPECTRAHSGKYSITAKNKAGQKTANCRVKVMDAPGPPKDLKVSDITRGSCRLSWKMPDDDGGDRIKGYVIEKKTIDGKAWTKVNPNCGSTTFVVPDLISEQQYFFRVRAENRFGIGPPAETIQRTTARDPIYPPDPPIKLKIGLITKNTVHLSWKPPKNDGGSPVTHYIVECLAWDPTGKKKEAWRQCNRRDVEELEFTVEDLIEGGEYEFRVKAVNEAGVSKPSATVGPVIVKDQTCPPAIELKEFMEVEEGTDVNIVAKIKGVPFPTLTWFKAPPKKPDSKEPVVYDTHVNKQVVDDTCTLVIPQSRRSDTGLYSITAVNNLGTASKEMRLNVLGRPGPPVGPIKFESISADQMTLSWLPPKDDGGSKITNYVIEKREANRKTWVRVSSEPKECMYTIPKLLEGHEYVFRIMAQNKYGIGEPLDSEPETARNLFSVPGAPDKPTVSSVTRNSMTVNWEEPEYDGGSPVTGYWLEMKDTTSKRWKRVNRDPIKAMTLGVSYKVTGLIEGSDYQFRVYAINAAGVGPASLPSDPVTARDPVAPPGPPFPKVTDWTKSSVDLEWSPPLKDGGSKITGYIVEYKEEGKEEWEKGKDKEVRGTKLVVTGLKEGAFYKFRVRAVNVAGVGEPGEVTDVIEMKDRIVSPDLQLDASVRDRIVVHAGGVIRIIAYVSGKPPPTVTWSMNERALPQEAAIETTAISSSMVIKNCQRSHQGVYSLLAKNEGGERKKTIIVDVLDVPGPVGIPFLSDNLTNDSCKLTWFSPEDDGGSPITNYVIQKREADRRAWTPVTYTVTRQNATVQGLIQGKSYFFRIAAENSIGMGPFVETPNALVIRDPITVPERPEDLEVKEVTKNTVSLTWNPPKYDGGSEIINYVLESRLIGTEKFHKVTNDNLLSRKYTVKGLKEGDTYEYRVSAVNIVGQGKPSFCTKPITCKDELAPPTLDLDFRDKLTVRVGESFALTGRYSGKPKPKIDWFKDEADVLEDDRTHIKTTPTTLALEKTKAKRSDSGKYCVVVENSTGSRKGFCQVNVVDRPGPPVGPVIFDEVTKEYMVISWKPPLDDGGSEITNYIIEKKELGKDIWMPVTSASAKTTCKVPKLLEGKDYIFRIHAENLYGISDPLVSDSMKAKDRFRVPDAPEQPVVTEVTKDSALVTWNKPNDGGKPITNYILEKRETMSKRWVRVTKEPIHPYTKYRVPDLLEGCQYEFRVSAENEIGIGDPSPPSKPVFARDPIAKPSPPINPEAIDTTCNSVELTWQPPRHDGGSKILGYIVEYQKVGDEEWRRANHTPESCPETKYKVTGLRDGQSYKFRVLAVNEAGESDPAHVPEPVLVKDRLEPPELILDANMAREQHIRVGDTLRLSAIIKGVPFPKVTWKKEDREAPTKAQIDVTPVGSKLEIRNAAHEDGGIYSLTVENPAGTKTVSVKVLVLDKPGPPRDLEVSEIRKDSCYLTWKEPLDDGGSVVTNYVVERKDVATAQWSPLSTTSKKKSHMAKHLTEGNQYLFRVAAENQYGRGPFVETPKPIKALDPLHPPGPPKDLHHVDVDKTEVSLVWNKPDRDGGSPITGYLVEYQEEGDKDWIKFKTVKNLDCVVTGLQQGKTYRFRVKAENIIGLGLPDTTIPIECQEKLVPPSVELDVKLIEGLVVKAGTTVRFPAIIRGVPVPTAKWTTDGTEIKTDDHYTVETDSFSSVLTIKNCLRKDTGEYQLTVSNAAGTKTVAVHLTVLDVPGPPTGPINILDVTPEYMTISWQPPKDDGGSPVINYIVEKQDTRKGTWGVVSAGSSKLKLKVPHLQKGCEYVFRVKAENKMGVGPPLDSIPTVAKHKFSPPSPPGKPVVTDITENAATVSWTLPKSDGGSPITGYYVERREITGKWVRVNKTPIADLKFRVTGLYEGNTYEFRVFAENLAGLSNPSPSSDPIKACRPIKPPGPPINPKLKDKSKESADLVWTKPLSDGGSPILGYVVEYQKPGTAQWDRINKDELIRQCAFRVPGLIEGNEYRFRIRAANIVGEGEPRELAESVIAKDILHPPEVELDVTCRDVITVRVGQTIRILARVKGRPEPDITWSKEGKVLVKDKRVDLIHDLPRVELQIKEAVRADHGKYIISAKNSSGHAQGSAIVNVLDRPGPCQNLKVSNVTKENCTISWENPLDNGGSEITNFIVEYRKPNQKGWSIVASDVTKRLVKANLLANNEYYFRVCAENKVGVGPTIETKTPILAINPIDRPGEPENLHIADKGKTFVYLKWRRPDYDGGSPNLSYHVERRLKGSADWERVHKGSIKETHYMVDKCVENQIYEFRVQTKNEGGESDWVRTEEVVVKEDLQKPVLDLKLSGVLTVKAGDTIRLEAGVRGKPFPEVAWTKDKDATDLTRSPRVKIDTSAESSKFSLTKAKRSDGGKYVITATNPAGSFVAYATVNVLDKPGPVRNLKITDVSSDRCTIRWDPPEDDGGCEIQNYILEKCESKRMVWSTYSANVLTPSATVTRLIEGNEYIFRVRAENKIGTGPPTESKPVIAKTKYDRPGRPDPPEVTKVSKEEMTVVWNAPEYDGGKSITGYYLEKKEKHAVRWVPVNKSAIPERRLKVQNLLPGHEYQFRVKAENEVGIGEPSLPSRPVVAKDPIEPPGPPTNFKVVDTTKSSITLAWGKPVYDGGAPIIGYVVEMRPKIADASPDEGWKRCNAAAQLIRMEFTVTSLDENQEYEFRVCAQNQVGIGRPAELKEAIKPKEILEPPEIDLDASMRKLVVVRAGCPIRLFAIVRGRPAPKVTWRKVGIDNVVRKGQVDLVDTMAFLVIPNSTRDDSGKYSLTLVNPAGEKAVFVNVKVLDTPGPVADLKVSDVTKTSCHVSWAPPENDGGSQVTHYIVEKREAERKTWSTVTPEVKKTSFNVTNLVPGNEYFFRVTAVNEYGPGVPTDIPKPVLASDPLSEPDPPRKLEVTEMTKNSATLAWLPPLRDGGAKIDGYIISYREEDQPADRWTEYSVVKDLSLIVTGLKEGKKYKFRVAARNAVGVSMPREAEGVYEAKEQLLPPKILMPEQITIKAGKKLRVEAHVYGKPNPICKWKKGDDEVVTSSHLAIHKADGSSVLIIKDVTRKDSGYYSLTAENSSGSDTQKIKVTVMDAPGPPQPPFDISEIDADACSLSWHIPLEDGGSNITNYIVEKCDVSRGDWVTALASVTKTSCRVGKLIPGQEYIFRVRAENRFGISEPLTSPKMLAKFPFDVPSEPKNARVTKVNKDCIFVAWDRPDSDGGSPITGYLIERKERNSLLWVKANDTIVRSTEYPCAGLVEGLEYSFRIYALNKAGSSPPSKPTEYVTARMPVDPPGKPEVVDVTKNSASLIWARPKHDGGSRIIGYFVEACKLPGDKWVRCNTTPHQIPQEEYTATGLEENAQYQFRAIAKTAVNISQPSEPSDPVTILAENVPPRIELSVEMKSLLTVKAGTNVCLDATVFGKPMPTVSWKKDTTPIKQAEGIKMAMKRNLCTLELFSVNRKDSGDYTITAENSSGSKSATIKLKVLDKPGPPASVKINKMYADRAMLSWEPPLEDGGSEITNYIIDKRETSRPNWAQVSATVPITSCTVEKLIEGHEYQFRICAENKYGVGDPILTEPAIAKNPYDPPGRCDPPVISNITKDHMTVSWKAPADDGGSPITGYLVEKRETQAVNWTKVNRKPVIERTLKATGLQEGTEYEFRVTAINKAGPGKPSDASKAVYAQDPLYPPGPPAFPKVYDTTRSSVSLSWGKPAYDGGSPIIGYLVEVKRADSDHWVRCNLPEKLQKTRFEVTGLMENTEYQFRVYAVNKIGYSDPSDVPDKHCPKDILIPPEGELDAELRKTLILRAGVTMRLYVPVKGRPPPKITWSKPNVNLRERIGLDIKSTDFDTFLRCENVNKYDAGKYILTLENSCGKKEYTIVVKVLDTPGPPVNVTVKEVSKDSAYVTWDPPIIDGGSPIINYVVEKRDAERKSWSTVTTECSKTSFRVSNLEEGKSYFFRVFAENEYGIGDPGETRDAVKASETPGPVVDLKALAITKSSCTIGWKKPRSDGGSRITGYVVDFLTEENKWQRVMKSLSLQYSTKDLKEGKEYTFRVSAENENGEGTPSEIVVVAKDDVVAPDLDLKDLPDLCYLAKENSNFRLKIPIKGKPAPSVSWKKGEDPLATDTRVSVESTAVNTTLVVYDCQKSDAGKYTITLKNVAGTKEGTLSIKVVGKPGIPTGPIKFDEVTAEAMTLKWGPPKDDGGSEITNYVLEKRDSVNNKWVTCASAVQKTTFRVTRLHEGIEYTFRVSAENKYGVGEGLKSEPIVAKHPFDVPDAPPPPNIVDVRHDSVSLTWTDPKKTGGSPITGYHIEFKERNSLLWKRANKTPIRMKDFKVTGLTEGLEYEFRVMAINLAGVGKPSLPSEPVVALDPIDPPGKPEVISVTRNSVTLIWTEPKYDGGHKLTGYIVEKRDLPSKSWMKANHVNVPDCAFTVTDLVEGGKYEFRIRAKNTAGAISAPSESTGTIICKDEYEAPTIVLDPTIKDGLTVKAGDSIVLSAISILGKPLPKSSWSRAGKDIRPSDIAQITSTPTSSMLTVKYATRKDAGEYTITATNPFGTKEEHVKVSVLDVPGPPGPIEISNVSAEKATLTWTPPLEDGGSPIKAYVLEKRETSRLLWTVVSEDIQACRHVVTKLIQGNEYLFRVSAVNHYGKGEPVQSEPVKMVDRFGPPGPPGKPEISNVTKNTATVSWKRPTDDGGSEITGYHVERREKKGLRWVRATKTPVSDLRCKVTGLQEGNTYEFRVSAENRAGIGPPSDASNPVLMKDVAYPPGPPSNAHVTDTTKKSASLAWGKPHYDGGLEITGYVVEHQKVGDDAWIKDTTGTALRITQFVVPDLQTKEKYNFRISAINDAGVGEPAVIPNVEIVEKEVAPDFELDAELRRTLVVRAGLSIRIFVPIKGRPAPEVTWTKDNINLKHRANIENTESFTLLIIPECNRYDTGKFVMTIENPAGKKSGFVNVRVLDTPGPVLNLRPTDITKDSVTLHWDLPLIDGGSRITNYIVEKREATRKSYSTVTTKCHKCTYKVTGLTEGCEYFFRVMAENEYGVGEPTETTEPVRASEAPLPPDSLNIMDITKNTVSLAWPKPRHDGGSKITGYVIEAQRKGSDQWTHISTVKGLECVVRNLTEGEEYTFQVMAVNSAGRSAPRESRPVIVKEQTMLPELDLRGIYQKLVIARAGDNIKVEIPVLGRPKPTVTWKKGDQILKQTQRVNVENTATSTILNINECVRSDSGPYPLTAKNTVGEVGDVITIQVHDIPGPPTGPIKFDEVSSDFVTFSWEPPENDGGVPISNYVVEMRQTDSTTWVELATTVIRTTYKATRLTTGVEYQFRVRAQNRYGVGPGITSASVVANYPFKVPGPPGTPQVTAVTKDSMTISWHEPLSDGGSPILGYHIERKERNGILWQTVSKALVPGNIFKSTGLTDGIAYEFRVIAENMAGKSKPSKPSEPMFALDPIDPPGKPVPLNITRHTVALKWAKPEYTGGFKITSYVVEKRDLPNGRWLKANFSNILENEFTVSGLTEDAAYEFRVIAKNAAGAISPPSEPSDAITCRDDLEAPRIMVDVRFKDTITLKAGEAFKLEADVSGRPPPTMEWTKDGKELEGTGKLEIKIADFSTHLINKDSSRTDSGAYILTATNPGGFAKHIFNVKVLDRPGPPEGPLAVSDVTSEKCVLSWLPPLDDGGAKIDHYIVQKRETSRLAWTNVATEVQVTKLKVTKLLKGNEYIFRVMAVNKYGVGEPLESEPVLAVDPYGPPDPPKNPEVTTITKDSMVVCWGHPDSDGGSEIINYIVERRDKAGQRWVKCNKKALTDLRFKVSGLTEGHEYEFRIMAENAAGISAPSATSPFYKACDTVFKPGPPGNPRVLDTSRSSISIAWNKPIYDGGSEITGYMVEIALPEEDEWQVVTPPAGLKATSYTITSLIENQEYKIRIYAMNSEGLGEPALVPGTPKAEERMLPPEIELDADLRKVVTIRACCTLRLFVPIKGRPAPEVKWAREHGESLDKASIESTSSYTLLVVGNVNRFDSGKYILTVENSSGSKSAFVNVRVLDTPGPPQNLKIKEVTKTSVTLTWEPPLLDGGSKIKNYIVEKRESTRKAYSTVATNCHKTSWKVDQLQEGCSYYFRVLAENEYGIGLPAETAESVKASERPLPPGKITLTDVTRNSVSLSWEKPEHDGGSRILGYIVEMQSKGSDRWATCATVKVTEATITGLIQGEEYSFRVSAQNEKGISDPRQLSVPVIAKDLVIPPAFKLLFNTFTVLAGEDLKIDVPFIGRPPPAVTWHKDDIPLKQTTRVNAESTENNSLLTIKEACREDVGHYTVKLTNSAGEATETLNVIVLDKPGPPTGPVKMDEVTADSVTLSWEPPKYDGGSSINNYIVEKRDTSTTAWQIVSATVARTTIKACRLKTGCEYQFRIAAENRYGKSTYLNSEPVVAQYPFKVPGPPGTPFVTLASKDSMEVQWHEPVSDGGSKVIGYHLERKERNSILWVKLNKTPIPQTKFKTTGLEEGIEYEFRVSAENIVGIGKPSKPSECYAAHDPCDPPGRPEAIIVTRNSVTLQWKKPTYDGGSKITGYIVEKKELPDGRWMKASFTNIIDTQFEVTGLLEDHRYEFRVIARNAAGVFSEPSESTGAITARDEVEPPRISMDPKYRDTVVVQAGESFKIDADIYGKPIPTTQWVKGDQELSSTARLEIKSTDFATSLSVKDAVRVDSGNYILKAKNVAGEKSVTINVKVLDRPGPPEGPVAISGVTAEKCTLAWKPPLQDGGSDITNYIVERRETSRLVWTLVDANVQTLSCKVLKLLEGNEYIFRIMAVNKYGVGEPLESESLIAKNPFVVPDAPKAPEVTAVTKDSMIVVWERPASDGGSEILGYVLEKRDKEGIRWTRCHKRLIGELRLRVTGLLENHNYEFRVSAENAAGLSEPSPPSAYQKACDPIYKPGPPNNPKVMDVTRSSVFLSWTKPIYDGGCEIQGYIVEKCDVSVGEWTMCTPPTGINKTNLEVEKLLEKHEYNFRICAINKAGVGEHADVPGPVMVEEKLEAPDIDLDLELRKVINIRAGGSLRLFVPIKGRPTPEVKWGKVDGDIRDAAIIDVTSSFTSLVLDNVNRYDSGKYTLTLENSSGTKSAFVTVRVLDTPSPPVNLKVTEITKDSVSITWEPPLLDGGSKIKNYIVEKREATRKSYAAVVTNCHKNSWKIDQLQEGCSYYFRVTAENEYGIGLPARTADPIKVAEVPQPPGKITVDDVTRNSVSLSWTKPEHDGGSKIIQYIVEMQAKNTDKWSECARVKSLDAVITNLTQGEEYLFRVIAVNEKGRSDPRSLAVPIIAKDLVIEPDVRPAFSSYSVQVGQDLKIEVPISGRPKPSISWTKDGMPLKQTTRINVTDSLDLTTLSIKETHKDDGGQYGITVSNVVGQKTAAIEIITLDKPDPPKGPVKFDEISAESITLSWNPPLYTGGCQITNYIVQKRDTTTTVWDVVSATVARTTLKVTKLKTGTEYQFRIFAENRYGQSFALESEPVVAQYPYKEPGPPGTPFVTAISKESMVVQWHEPINNGGSPVIGYHLERKERNSILWTKVNKTIIHDTQFKALNLEEGIEYEFRVYAENIVGVGKASKNSECYVARDPCDPPGTPEAIIVKRNEITLQWTKPVYDGGSMITGYIVEKRDLPEGRWMKASFTNVIETQFTVSGLTEDQRYEFRVIAKNAAGAISKPSDSTGPITAKDEVELPRISMDPKFRDTIVVNAGETFRLEADVHGKPLPTIEWLRGDKEIEESARCEIKNTDFKALLIVKDAIRIDGGQYILRASNVAGSKSFPVNVKVLDRPGPPEGPVQVTGVTAEKCTLAWSPPLQDGGSDISHYVVEKRETSRLAWTVVASEVVTNSLKVTKLLEGNKYIFRIMAVNKYGVGEPLESAPVLMKNPFVLPGPPKSLEVTNIAKDSMTVCWNRPDSDGGSEIIGYIVEKRDRSGIRWIKCNKRRITDLRLRVTGLTEDHEYEFRVSAENAAGVGEPSPATVYYKACDPVFKPGPPTNAHVVDTTKNSITLAWSKPIYDGGSEILGYVVEICKADEEEWQIVTPQTGLRVTRFEIAKLIEHQEYKIRVCALNKVGLGEAASVPGTVKPEDKLEAPELDLDSELRKGIVVRAGGSARIHIPFKGRPTPEITWSKEEGEFTDKVQIEKGINFTQLSIDNCDRNDAGKYILKLENSSGSKSAFVTVKVLDTPGPPQNLAVKEVRKDSVLLVWEPPIIDGGAKVKNYVIDKRESTRKAYANVSSKCNKTSFRVENLTEGAIYYFRVMAENEFGVGVPTETSDAVKASEPPSPPGKVTLTDVSQTSASLMWEKPEHDGGSRILGYVVEMQPKGTEKWSVVAESKVCNAVVSGLSSGQEYQFRVKAYNEKGKSDPRVLGIPVIAKDLTIQPSFKLPFNTYSVQAGEDLKIEIPVIGRPRPKISWVKDGEPLKQTTRVNVEETATSTILHIKESSKDDFGKYSVTATNSAGTATENLSVIVLEKPGPPVGPVKFDEVSADFVVISWEPPAYTGGCQISNYIVEKRDTTTTNWQMVSATVARTTIKISKLKTGTEYQFRIFAENRYGKSTPLDSKPVVVQYPFKEPGPPGTPFVTSISKDQMLVQWHEPVNDGGSKVTGYHLEQKEKNSILWVKLNKIPIQDTKFKTTGLDEGLEYEFRVSAENIVGIGKPSKVSECYVARDPCDPPGRPEAIVITRNSVTLKWKKPVYDGGSKITGYIVEKKDLPDGRWMKASFTNVVETEFTVTGLVEDQRYEFRVIARNAADNFSEPSESSGAITARDEIDAPNASLDPKYRDVIIVHAGETFVLEADIRGKPIPDIIWSKDGNELEETAARMEIKSTLQKTTLIVKDCIRTDGGQYTLKLSNVGGTKTIPITVKVLDRPGPPEGPLKVTGVTAEKCYLAWNPPLQDGGASISHYIIEKRETSRLSWTQVSNEVQALNYKVTKLLPGNEYIFRVMAVNKYGIGEALESEPVIACNPYKRPGPPSTPEASAITKDSMVLTWTRPVDDGGAEIEGYILEKRDKEGIRWTKCNKKTLTDLRFRVTGLTEGHSYEFRVAAENAAGVGEPSEPSVFYRACDALYPPGPPSNPKVTDTSRSSVSLAWNKPIYDGGAPVRGYVIELKKAAADEWTTCTPPSGLQGKQFTVTKLKENTEYNFRICAFNTEGVGEPATIPGSVVAQERMEAPEIELDADLRKVVTLRASATLRLFVTIKGRPEPEVKWEKAEGILTERAQIEVTSSYTMLVIDNVTRFDSGRYNLTLENNSGSKTAFVNVRVLDSPSAPVNLTIREVKKDSVTLSWEPPLIDGGAKITNYIVEKRETTRKAYATITNNCTKNTFKIENLQEGCSYYFRVLASNEYGIGLPAETAEPVKVSEPPLPPGRVTLVDVTRNTATIKWEKPESDGGSKITGYVVEMQTKGSEKWSACTQVKTLETTISGLTAGEEYVFRVAAVNEKGRSDPRQLGVPVIAKDIEIKPSVELPFNTFNVKANDQLKIDIPFKGRPQATVAWKKDGQVLRETTRVNVASSKTVTTLSIKEASREDVGTYELCVSNTAGSITVPITVIVLDRPGPPGPIRIDEVSCDNVSISWNPPEYDGGCQISNYIVEKRETTSTTWQVVSQAVARTSIKIVRLTTGSEYQFRVCAENRYGKSSYSESSAVVAEYPFSPPGPPGTPKVVHATKSTMVVSWQVPVNDGGSQVIGYHLEYKERSSILWSKANKVLIADTQMKVSGLDEGLMYEYRVYAENIAGIGKCSKACEPVPARDPCDPPGQPEVTNITRKSVSLKWSKPRYDGGAKITGYIVERRELPDGRWLKCNFTNVQETYFEVTELTEDQRYEFRVFARNAADSVSEPSESTGPITVKDDVEAPRIMMDVKFRDVIIVKAGEVLKINADIAGRPLPVISWAKDGVEIEERAKTEIVSTDYTTTLTVKDCVRRDTGQYVLTLKNVAGTRTMAVNCKVLDKPGPPAGPLEINGLTAEKCSLSWGRPQEDGGADIDYYIVEKRETSRLAWTICEAELRTTSCKVTKLLKGNEYIFRVTGVNKYGVGEPLESMAVKALDPFTTPSPPTSLEITSVTKDSMTLCWSRPETDGGSDISGYIIERREKNSLRWMRVNKKPVYDLRVKSTGLREGCEYEYRVFAENAAGLSLPSETSPLVRAEDPVFLPSPPSKPKIVDSGKTTITIGWVKPLFDGGAPITGYTVEYKKSEETDWKVAIQSFRGTEYTMSGLTTGDEYVFRVRSLNKMGASDPSDSSDPQVAKEREEEPVFDVDSEMRKTLIVKAGSSFTMTVPFRGRPIPNVSWSKPDTDLRTRAYIDSTDSRTLLTIENANRNDSGKYTLTIQNVLSAASMTFVVKVLDSPGPPANITVREVTKETAMLSWDVPENDGGAPVKNYHIEKREASKKAWVSVTNNCNRLSYKVTNLQEGAIYYFRVSGENEFGVGVPAETKEGVKITEKPSPPEKLGVTSVSKDSVSLSWLKPEHDGGSRIIHYVVEALEKGQKTWVKCAVVKTTHHVVSGLRESHEYFFRVFAENQAGLSDPRELLLPVLIKDQLEPPEIDMKNFPSHTVYVRAGSNLKVDIPISGKPLPKVTLSRDGVPLKATMRFNTEITAENLTINLKESVTTDAGRYEITAANSSGTTKTFINIIVLDRPGPPTGPVAISDITEESVTLKWEPPKYDGGSHVTNYIVLKRETSTAVWSEVSATVARTMIKVMKLTTGEEYQFRIKAENRFGISDHIDSVCVVVKLPYTTPGPPSTPWVSNVTRESITVGWHEPVSNGGSAVTGYHLEMKDRNSILWQKANKMIIRTTHFKVTTISAGLIYEFRVYAENAAGIGKPSHPSEPVLAIDACEPPRNVRITDISKNSVNLSWQQPAFDGGSKITGYIVERRDLPDGRWTKASFTNVIETQFTVSGLTQNSQYEFRVFARNAVGSVSNPSEVVGPITCIDSYGGPVIDLPLEYTEVVKYRAGTSVKLRAGISGKPEPTIEWYKDDKELQTNALVCVENSTDLASILIKDANRLNSGSYELKLRNAMGSASATIRVQILDKPGPPGGPIEFKTVTAEKITLLWRPPADDGGAKITHYIVEKRETSRVVWSMVAENLEECIVTTTKIIKGNEYVFRVRAVNKYGIGEPLESEPVVAKNAFVTPGPPSIPEVTKITKNSMTVVWDRPTVDGGSEINGYFLERRDKKSLAWLKVLKETIRDTRQKVTGLTENSDYQYRVCAVNAAGVGPFSEPSDFYKAADPIDPPGPPAKIRIADSTKSSITLGWSKPVYDGGSDVTGYVVEMKQGDEEEWTIVSTRGEVRTTEYVVSNLKPGVNYYFQVSAVNCAGQGEPITMTEPAQAKDVLEEPEIDLDVALRTSVIAKAGEDVQLLIPFKGRPPPTVTWRKDEKNLGSDTRYSIQNTDSSSLLVIPQVTRNDTGKYILTIENGVGQPKSSTVSVKVLDTPAACQKLQVKHVSLGTVTLLWDPPLIDGGSPIINYVIEKRDATKRTWSVVSHKCSGTSFKVTDLSEKTPFFFRVLAENEIGIGEPCETTEPVKAAEVPAPIRDLSMKDSTKTSVVLSWTKPDFDGGSIITDYLVERKGKGEQAWSHAGISKTCEIEIGQLKEQSVLEFRVSARNEKGQSDPVTIGPLTVKELVITPEVDLSEIPGAQISVRIGHNVHLELPYKGKPKPSISWLKDGLPLKESEYVRFSKTENKITLSIKNSKKEHGGKYTVILDNAVCRNSFPITIITLGPPSKPKGPIRFDEIKADSAIMSWDIPEDDGGGEITCYSIEKREASQTNWKMVCSSVARTTFKVSNLVKDSEYQFRVRAENRYGVSEPLASNIIVAKHQFRIPGPPGKPVIYNVTSDGMSLTWDAPVYDGGSEVTGFHVEKKERNSILWQRVNTSPISGREYRATGLIEGLDYQFRVYAENSAGLSSPSDPSKFTLAVSPVDPPGTPDYIDVTRETITLKWNPPLRDGGSKIVAYSIEKRQGSDRWVRCNFTDVSECQYTVTGLSPGDRYEFRIIARNAVGTISPPSQSSGLIMTRDENVPPTVEFGPEYFDGLVIKSGDSLRIKALVQGRPVPRVTWFKDGVEIERRMNMEITDVLGSTSLFVRDATRDHRGVYTVEAKNVSGSTKAEVTVKVQDTPGKVVGPIRFTNITGEKMTLWWEAPLNDGCAPVTHYIIEKRETSRLAWALIEDNCEALSYTAIKLITGNEYQFRISAVNKFGVGRPLESDPVVAQIQYTIPDAPGVPEPSNVTGNSITLTWTRPESDGGSEIQHYILERREKKSTRWVKVISKRPISETRFKVTGLVEGNEYEFHVMAENAAGIGPASGISRLIKCREPVNPPSAPSVVKVTDTSKTTVSLEWARPVFDGGMEIIGYIIEMCKADLGDWHKVNTEPCVKTRYTVTDLQAGEEYKFRVSAVNGAGKGDSCEVTGTIKAVDRLSAPELDIDANFKQTHIVRAGVSIRLFIAYQGRPTPTAVWSKPDSNLSIRADIHTTDSFSTLTVENCNRNDAGKYTLTVENNSGKKSITFTVKVLDSPGPPGPITFKDVTRGSATLMWDAPLLDGGARIHHYVIEKREASRRSWQVVSEKCTRQILKVSELTEGVPYYFRVSAENEYGVGEPYEMPEPIVATEQPAPPRRLDVVDTSKSSAVLAWLKPDHDGGSRITSYLLEMRQKGSDFWVEAGHTKQLTFTVERLVENTEYEFRVKAKNDAGYSEPREAFSSVIIKEPQIEPTADLTGITNQLITCKAGSTFTIDVPISGRPAPKVTWKLEEMRLKETDRMSIATTKDRTTLTVKDSMRGDSGRYFLTLENTAGVKTFTITVVVIGRPGPVTGPIEVSSVSAESCVLSWTEPKDDGGTEITNYIVEKRESGTTAWQLINSSVKRTQIKVTHLTKYKEYCFRVSSENRFGVSKPLESVPIVAEHPFVPPSAPTRPEVYYVSANAMSIRWEEPYHDGGSKIVGYWVEKKERNTILWVKENKVPCLECNYKVTGLVEGLEYQFRTYALNAAGVSKASEASRPIMAQNPVDPPGRPEVTDVTRSTVSLIWSAPVYDGGSKVVGYIIERKPVSEVGDGRWLKCNYTIVSDNFFTVTALSEGDTYEFRVLAKNAAGVISKGSESTGPVTCRDEYAPPKAELDARLQGDLVTIRAGSDLVLDAAVGGKPEPKIIWTKGDKELDLCEKISLQYTGKRATAVIKYCDRSDSGKYTLTVKNASGTKSVSVMVKVLDSPGPCGKLTVSRVTEEKCTLAWSLPQEDGGAEITHYIVERRETSRLNWVIVEGECLTASYVVTRLIKNNEYTFRVRAVNKYGLGVPVESEPIVARNSFTIPSQPGIPEEVGAGKEHIIIQWTKPESDGGNEISNYLVDKREKKSLRWTRVNKDYVVYDTRLKVTSLMEGCDYQFRVTAVNAAGNSEPSEASNFISCREPSYTPGPPSAPRVVDTTKRSISLAWTKPMYDGGTDIIGYVLEMQEKDTDQWCRVHTNATIRNNEFTVPDLKMGQKYSFRVAAVNAKGMSDYSETTAEIEPVERLEIPDLELADDLKKTVIVRAGASLRLMVSVSGRPSPVITWSKKGIDLANRAIIDNTESYSLLIVDKVNRYDAGKYTIEAENQSGKKSATVLVKVYDTPGPCPSVSVKEVSRDSVTITWEIPTIDGGAPVNNYIIEKREAAMRAFKTVTTKCSKTLYRISGLVEGTMYYFRVLPENIYGIGEPCETSDAVLVSEVPLVPTKLEVVDVTKSTVTLAWEKPLYDGGSRLTGYVLEACKAGTERWMKVVTLKPTVLEHTVISLNEGEQYLFRVRAQNEKGVSEPREIVTPVTVQDLRVLPTIDLSTMPQKTIHVPAGRPIELVIPITGRPPPTASWFFAGSKLRESERVTVETHTKVTKLTIRETTIRDTGEYTLELKNVTGTTSETIKVIILDKPGPPTGPIKIDEIDATSVTISWEPPELDGGAPLSGYVVEQRDAHRPGWLPVSESVTRPTFKFTRLTEGNEYVFRVAATNRFGIGSYLQSEVIECRSSISIPGPPETLQIFDVSRDGMTLTWYPPEDDGGSQVTGYIVERKEVRADRWVRVNKVPVTMTRYRSTGLIEGLEYEHRVTAINARGTGKPSRPSKPTVAMDPIAPPGKPQNPRVTDTTRTSVSLAWSVPEDEGGSKVTGYLIEMQKVDQREWTKCNTTPTKIREYTLTHLPQGAEYRFRVLACNAGGPGEPAEVPGTVKVTEMLEYPDYELDERYQEGVFVRQGGVIRLTIPIKGKPFPVCKWTKEGQDISKRAMIATSETHTELVIKEADRNDSGTYDLVLENKCGKKTVYIKVKVIGSPNTPEGPLEYDDIQARSVRVSWRPPADDGGADILGYILERREVPKAAWYTIDSRVRGTSLVVKGLKENVEYHFRVSAENQFGISKPLKSEEPVIPKTPLNPPEPPSNPPEVLDVTKSSVSLSWSRPKDDGGSRVTGYYIERKETSTDKWVRHNKTQITTTMYTVTGLVPDAEYQFRIIAQNDVGLSETSPASEPVVCKDPFDKPSQPGELEILSISKDSVTLQWEKPECDGGKEILGYWVEYRQSGDSAWKKSNKERIKDRQFTIGGLLEATEYEFRVFAENETGLSRPRRTAMSVKTKLTSGEAPGVRKEMADVTTKLGEAAQLSCQIVGRPLPDIKWYRFGKELIQSRKYKMSSDGRTHTLTVMTDEQEDEGVYTCVATNEVGEVESSSKLLLQAAPQFHPGYPLKEKYYGAVGSTLRLHVMYIGRPVPAMTWFHGQKLLQNSEKITIENTEHYTHLVMKNVQRKTHAGKYKVQLSNAFGTVDATLDVEIQDKPDKPTGPIVIEALLKNSVVISWKPPADDGGSWITNYVVEKCEAKEGAEWQLVSSAISVTTCRIVNLTENAGYYFRVSAQNTFGISEPLEVASIVIIKSPFEKPGVPGKPTITAVTKDSCVVAWKPPASDGGAKIRNYYLERREKKQNKWIAVTTEEIRETVFSVQNLIEGLEYEFRVKCENLGGESEWSEISEPVTPKSDVPIQAPHFKEELRNLNVRYQSNATLVCKVTGHPKPIVKWYRQGKEIIADGLKYRIQEFKGGYHQLIIASVTDDDATVYQVRATNQGGSVSGTASLEVEVPAKIHLPKTLEGMGAVHALRGEVVSIKIPFSGKPDPVITWQKGQDLIDNNGHYQVIVTRSFTSLVFSNGVERKDAGFYVVCAKNRFGIDQKTVELDVADVPDPPRGVKVSDVSRDSVNLTWTEPASDGGSKVTNYIVEKCATTAERWLRVGQARETRYTVINLFGKTSYQFRVIAENKFGLSKPSEPSEPTVTKEDKTRAMNYDDEVDETREVTTTKASHSKTKELYEKYMIAEDLGRGEFGIVHRCVETSSKRTFMAKFVKVKGTDQVLVKKEISILNIARHRNILYLHESFESMEELVMIFEFISGLDIFERINTSAFELNEREIVSYVRQVCEALEFLHSQNIGHFDIRPENIIYQTRKNSTIKIIEFGQARQLKPGDNFRLLFTAPEYYAPEVHQHDVVSTATDMWSLGTLVYVLLSGINPFLAETNQQMIENIMNAEYTFDEEAFKEISLEAMDFVDRLLVKERKSRMTASEALQHPWLKQRIDRVSTKVIRTLKHRRYYHTLIKKDLNMVVSAARISCGGAIRSQRGVSVAKVKVASIEIGPVSGQIMHAIGEEGGYVKYVCKIENYDQSTQVTWYFGVRQLENSEKYEITYEDGVATMYVKDITKFDDGTYRCKVVNDYGEDSSYAELFVKGVREVYDYYCRRTKKVKRRTDAMRLLERPPEFTLPLYNKTAYVGENVRFGVTITVHPEPRVTWYKSGQKIKPGDDEKKYTFESDKGLYQLTINSVTTDDDAEYTVVARNKHGEDSCKAKLTVTLHPPPTETTLRPMFKRLLANAECHEGQSVCFEIRVSGIPAPTLKWEKDGQPLSLGPHIEIVHEGLDYYALHIRDTLPEDTGYYRVTATNTAGSTSCQAHLQVERLRYVKQEFQSKEERERHVQKQIDKTLRMAEILSGTETVPLTPVAQEALREAAILYKPAVSTKTVKGEYRLQTEEKKEERKLRMPYEVPEPRRFKQATVEEDQRIKQFVPMSDMKWYKKIRDQYEMPGKLDRVVQKRPKRIRLSRWEQFYVMPLPRITDQYRPKWRIPKLTQDDLEMVRPARRRTPSPDYDLYYYRRRRRSLGDMSDEELLLPIDDYLAMKRTEEERLRLEEELELGFSASPPSRSPPRFELSSLRYSSPPAHVKVEDRRRDFRYSTYHVPTKEETSTSYAELRERHAQASYRQPKLRQRIMAEKEEEELLRPVTTTQRLSEYKSELDYMSKEEKSKKKSKRQRQVTEITEIEEEYEISRRAQRESSSSVSRLLRRRRSLSPTYIELMRPVSELIRSHPRPAEEYEDDAERRSPTPERTRPRSPSPVSSERSLSRFERSARFDIFSRYESMKAALKTQKTSERKYEVLSQQPFTLDHAPRITLRMRSHRVPCGQNTRFILNVQSKPTAEVKWYHNGVELQESSKIHYTNTSGVLTLEILDCQTEDGGTYRAVCTNYKGEASDYATLDVTGGAYTTYASQRRDEEVPKSVFPELTKTEAYAVSSFKRTSELEAASSVREVKSQMTETRESLSTYEHYASAEMKSATSEEKSLEEKATVRKIKTTLAARILTKPRSITVHEGESARFSCDTDGEPVPTVTWLREGQVVSTSARHQVTTTKYKSTFEISSVQASDEGNYSVVVENSDGKQEAQFTLTVQKARVIEKAVTSPPRVKSPEPRVKSPETVKSPKRVKSPEPVTSHPKAVSPTETKPTEKGQHLPVSAPPKITQSLKAEASKDIAKLTCAVESSALCAKEVAWYKDGKKLKENGHFQFHYSADGTYELKIHNLSESDCGEYVCEVSGEGGTSKTSFQFTGQSFKSIHEQVSSISETTKSVQKTAESPEAKKQEPIAPESISSKPVIVTGLRDTTVSSDSVAKFTIKVTGEPQPTITWTKDGKAIAQGSKYKLSSNKEEFILEILKTETSDGGLYACTVTNSAGSVSSSCKLTIKAVKDTEAQKVSTQKTSEVTSQKKASAQEEISQKALTSEEIKMSEVKSHETLAIKEEASKVLIAEEVKRSAAASLEKSIVHEEVTKTSQASEEVKTHAEIKTLSTQMNITKGQRATLKANIAGATDVKWVLNGTELPNSEEYRYGVSGSDQTLTIKQASHREEGILSCIGKTSQGVVKCQFDLTLSEELSDAPSFITQPRSQNINEGQNVLFSCEVSGEPSPEIEWFKNNLPISISSNISVSRSRNVYTLEIRNAAVSDSGKYTIKAKNFHGQCSATASLTVLPLVEEPPREVVLKTSSDVSLHGSVSSQSVQMSASKQEASFSSFSSSSASSMTEMKFASMSAQSMSSMQESFVEMSSSSFMGKSSMTQLESSTSRMLKAGGRGIPPKIEALPSDISIDEGKVLTVACAFTGEPTPEITWSCGGRKIQNQEQQGRFHIENTDDLTTLIIMDVQKQDGGLYTLSLGNEFGSDSATVNINIRSM

SEQ ID NO: 2 - protein, 1939 aa, Mus musculus
MSSDAEMAVFGEAAPYLRKSEKERIEAQNKPFDAKSSVFVVDAKESYVKATVQSREGGKVTAKTEGGATVTVKDDQVFSMNPPKYDKIEDMAMMTHLHEPAVLYNLKERYAAWMIYTYSGLFCVTVNPYKWLPVYNPEVVAAYRGKKRQEAPPHIFSISDNAYQFMLTDRENQSILITGESGAGKTVNTKRVIQYFATIAVTGDKKKEEATSGKMQGTLEDQIISANPLLEAFGNAKTVRNDNSSRFGKFIRIHFGATGKLASADIETYLLEKSRVTFQLKAERSYHIFYQIMSNKKPELIEMLLITTNPYDFAYVSQGEITVPSIDDQEELMATDTAVDILGFSADEKVAIYKLTGAVMHYGNMKFKQKQREEQAEPDGTEVADKAAYLTSLNSADLLKALCYPRVKVGNEYVTKGQTVQQVYNSVGALAKSMYEKMFLWMVTRINQQLDTKQPRQYFIGVLDIAGFEIFDFNTLEQLCINFTNEKLQQFFNHHMFVLEQEEYKKEGIDWEFIDFGMDLAACIELIEKPMGIFSILEEECMFPKATDTSFKNKLYEQHLGKSNNFQKPKPAKGKAEAHFSLVHYAGTVDYNIIGWLDKNKDPLNETVVGLYQKSGLKTLAFLFSGGQAAEAEGGGGKKGGKKKGSSFQTVSALFRENLNKLMTNLKSTHPHFVRCLIPNETKTPGAMEHELVLHQLRCNGVLEGIRICRKGFPSRILYADFKQRYKVLNASAIPEGQFIDSKKASEKLLGSIDIDHTQYKFGHTKVFFKAGLLGTLEEMRDEKLAQLITRTQAVCRGYLMRVEFKKMMERRESIFCIQYNVRAFMNVKHWPWMKLYFKIKPLLKSAETEKEMANMKEDFEKAKEDLAKSEAKRKELEEKMVALMQEKNDLQLQVQAEADGLADAEERCDQLIKTKIQLEAKIKELTERAEDEEEINAELTAKKRKLEDECSELKKDIDDLELTLAKVEKEKHATENKVKNLTEEMAGLDENIAKLTKEKKALQEAHQQTLDDLQAEEDKVNTLTKAKTKLEQQVDDLEGSLEQEKKLRMDLERAKRKLEGDLKLAQESTMDIENDKQQLDEKLKKKEFEMSNLQSKIEDEQALGMQLQKKIKELQARIEELEEEIEAERASRAKAEKQRSDLSRELEEISERLEEAGGATSAQIEMNKKREAEFQKMRRDLEEATLQHEATAAALRKKHADSVAELGEQIDNLQRVKQKLEKEKSELKMEIDDLASNMETVSKAKGNLEKMCRTLEDQLSEVKTKEEEQQRLINELSTQKARLHTESGEFSRQLDEKDAMVSQLSRGKQAFTQQIEELKRQLEEESKAKNALAHALQSARHDCDLLREQYEEEQEAKAELQRAMSKANSEVAQWRTKYETDAIQRTEELEEAKKKLAQRLQDAEEHVEAVNSKCASLEKTKQRLQNEVEDLMIDVERSNAACAALDKKQRNFDKVLAEWKQKYEETQAELEASQKESRSLSTELFKVKNAYEESLDQLETLKRENKNLQQEISDLTEQIAEGGKHIHELEKIKKQIDQEKSELQASLEEAEASLEHEEGKILRIQLELNQVKSEIDRKIAEKDEEIDQLKRNHLRVVESMQSTLDAEIRSRNDALRIKKKMEGDLNEMEIQLNHANRQAAEAIRNLRNTQGMLKDTQLHLDDALRGQDDLKEQLAMVERRANLMQAEIEELRASLEQTERSRRVAEQELLDASERVQLLHTQNTSLINTKKKLETDISQIQGEMEDIVQEARNAEEKAKKAITDAAMMAEELKKEQDTSAHLERMKKNMEQTVKDLQHRLDEAEQLALKGGKKQIQKLEARVRELENEVENEQKRNIEAVKGLRKHERRVKELTYQTEEDRKNVLRLQDLVDKLQTKVKAYKRQAEEAEEQSNVNLAKFRKIQHELEEAEERADIAESQVNKLRVKSREVHTKVISEE

SEQ ID NO: 3 - protein, 7152 aa, Mus musculus
MADEEEYEEVIEYYTEETVYEEVPGETITEVYETTTTRTSDYGQSETATPALAQPQPAKPVERKKVIRKKVDSSKFMTPYIQHSQKMQDLFSSNKYKENYEKAKGQPYAITSDTPELRRIKKVQDQLSEVKYRMDGDVAKTICHVDEKAKDIEHAKKVSQQVSKVLYKQNWEDTKDKYLLPPDAPELVQAVKNTAMFSKKLYTEDWEADKALFYPYNDSPELRRVAQAQKALSDIAYKKGLTEQQTQFTSLPDPPEVEFAKKVTNQVSKQKYKEDYEKKVKGKWSETPCFEIATARMNADNLSSRKYQEDFENMKDQIYFMQTETPEYKMNKQAGVAASKVKYKQDYEKNKGKADYNVLPASENPLLRQLKTAGNVLSDKLYKENYEKTKARSINYCETPKFQLDTVLQNFSSDTKYKDSYLKNILGHYVGSFEDPYHTHCMKVTAQNSDKNYKAEYEEDRGKGFFPQTITQEYEAIKKLDQCKDHAYKVHPDKTKFTQVTDSPVLVQAQVNSKQLSDLNYKAKHESEKFKCHIPPDAPAFIQHRVNAYNLSDNVYKQDWEKSKAKKFDIKVDAIPLLAAKANSKNASDVMYKKDYEKSKGKMIGALSINDDPKMLHSLKTAKNQSDRLYRENYEKTKAKSMNYCETPKYQLDTLLKNFSEAKYKDSYVKNMLGHYVGSFEDPYHTHCMKVSAQNSDKSYKAEYEEDKGKCYFPQTITQEYESIKKLDQCKDHTYKVHPDKTRFTAVTDTPVLLQAQLNTKQLSDLNYKAKHEGEKFKCSVPADAPEFIQHRVNAYNLSDNVYKHDWEKSKAKKFDIKVDAIPLLAAKANSKNASDVMYKKDYEKSKGKMIGALSINDDPKMLHSLKTAKNQSDREYRKDYEKSKTIYTAPLDMIQVTQAKKSQAIASDVDYKHVLHNYSYPPDSINVDLAKKAYALQSDVEYKADYNSWMKGCGWVPFGSLEMEKAKRASEILNEKKYRQHPDTLKFTSIEDAPITVQSKINQAQRSDIAYKAKGEEILHKYNLPADLPQFIQAKVNAYNISENMYKADLKDLSKKGYDLRIDAIPIKAAKAARQAASDIHYKKDYEKAKGKMVGFQSLQDDPKLVHYMNVAKIQSDREYKKDYEKTKTKYNTPHDMFDVVAAKKAQDVISNTNYKHSLHHYTYLPDAMDLELSKNMMHIQSDNAYKEDYNTWMKGIGWIPIGSLDQEKVKKAGDALSEKKYRQHPDTLKFTSIVDSPVMVQAKQNTQQASDILYKAKGEDVKHKYTLNPDLPQFLQAKCNAYNISDVCYKRDWHDLIAKGNNVLADAIPITAAKSSRNIASDYKYKEAYEKAKGKHVGFRSLQDDPKLVHYMNVAKLQSDREYKKNYENTKTSYHTPGDMVSITAARMAQDVATNVNYKQPLHHYTYLPDALSLEHTRNANQIQSDNVYKDEYNSFFKGIGWIPIGSLEVEKAKKAGEALNERKYRQHPDTIKFTSVPDSMGMVLAQQNTKQLSDLNYKVEGEKMKHKYTMDPELPQFIQAKVNAINMSDAHYKADWKKTLAKGYDLRPDAIPIVAAKSSRNIASDCKYKEAYEKARGKQIGFLSLQDDPKLVHYMNVAKIQSDREYKKGYEASKTRYHTPLDMFSVTAAKKSQEVATNSNYRQPYHNYTLLPDALNVEHSRNAMQIQSDNLYKSDFTHWMKGIGWVPIDSLEVEKAKKAGEILSERKYRQHPEKLKFTYSMDTMEQALNKSNKLTMDKRLYTEKWNKDKTSIHVMPDTPDILLSRVNQITMSDKLYKAGWEEEKTKGYDLKPDAIAIKAAKASRDIASDYKYKQAYEQSKGKHIGFRSLEDDPKLVHFMQVAKIQSDREYKKGYEKSKTSFHTPVDMFSVVAAKKSQEVATNANYRNIIHTYNMLPDAMSFELAKNMMQIQSDNQYKADYADFMKGIGWLPLGSLEAEKNKKAMEIISEKKYRQHPDTLKYSTLMDSMNMVLAQNNAKIMNKNLYKQAWEADKTKIHIMPDIPEIVLAKANAINMSDKLYKLSLEESRKKGYDLRTDAIPIKAAKASRDIASDYKYKHSYEKEKGKMVGFRSLEDDPKLVHSMQVAKMQSDREYKKNYEKTKTSYHTPADMLSVTAAKDAQANITNTNYKRLIHKYILLPDAMNIELTRNMNHIQSDNEYKQDYREWYRGLGWSPAGSLEVEKAKKATEYASDQKYRQHPSTFQFKKLTDSMDMVLAKQNAHTMNKHLYTIDWNKDKTKIHVMPDTPDILQAKQNQTLYSQKSYRLGWEEALKKGYDLPVDAISVQLAKTSRDIASDFKYKQGYRKQLGHHIGFLSVQDDPKLVLSMNVAKMQSDREYKKDFEKWKTKYTSPVDMLGVVLAKKCQALVSDADYRNYLHQWTCLPDQNDVIQAKKVYELQSENMYKSDLEWLRGIGWSPLGSLEAEKNKRASEIISEKKYRQPPDRNKFTSIPDAMDIVLAKTNAKNRSDRLYREAWDKDKTQIHIMPDTPDIILAKANLINTSDKLYKMGYEELKKKGYDLPVDAIPIKAAKASREIASEYKYKEGFRKQLGHHIGARNIKDDPKMMWSMHVAKIQSDREYKKDFEKWKTKYSSPVDMLGVVLAKKCQTLVSDADYRNYLHQWTCLPDQNDVIHARQAYDLQSDNVYKADLQWLKGIGWIPSGSLEDEKNKRATQILSDHVYRQHPNKFKFSSLMDSMPMVLAKNNAITMNHHLYTEAWNKDKTTVHIMPDTPEVILAKQNKVNYSEKLYKLGLEEAKRKGYDMRLDAIPIKTAKASRDIASDFKYKEGYRKQLGHHIGARGIHDDPKMMWSMHVAKIQSDREYKKDFEKWKTKYSSPVDMLGVVLAKKCQTLVSDADYRNYLHQWTCLPDQNDVIHARQAYDLQSDNMYKSDLQWMRGIGWVPIGSLDVEKCKRATEILSDKIYRQPPDKFKFTSVTDSLEQVLAKNNAITMNKRLYTEAWDKDKTQIHIMPDTPEIMLARMNKVNYSESLYKLANEEAKKKGYDLRSDAIPIVAAKASRDIASDYKYKDGYRKQLGHHIGARNIKDDPKMMWSMHVAKIQSDREYKKDFEKWKTKYSSPVDMLGVVLAKKCQILVSDIDYKHPLHEWTCLPDQNDVIHARQAYDLQSDNVYKSDLQWMRGIGWVPIGSLDVVKCKRAAEILSDNIYRQPPNKFKFTSVTDSLEQVLAKSNALNMNKRLYTEAWDKDKIQIHVMPDTPEIMLARQNRINYSESLYKLANEEAKKKGYDLRVDAIPIVAAKASRDIASDYKYKVGYRKQLGHHIGARNIEDDPKMMWSMHVAKIQSDREYKKDFEKWKTKYSSPVDMLGVVLAKKCQTLVSDVDYRNYLHQWTCLPDQNDVIHARQAYDLQSDNVYKSDLQWMRGIGWVPIGSLDVVKCKRAAEILSDNIYRQPPNKFKFTSVTDSLEQVLAKSNALNMNKRLYTEAWDKDKTQIHIMPDTPEIMLARQNKINYSESLYRQAMEEAKKEGYDLRSDAIPIVAAKASRDIASDYKYKEAYRKQLGHHIGARAIHDDPKMMWSLHIAKVQSDREYKKEFEKYKTRYSSPVDMLGIVLAKKCQTLVSDVDYKHPLHEWTCLPDQNDVIQARKAYDLQSDNLYKADLEWMKGIGWVPIGSLEVVKAKRATEILSDNIYRQRPDTLKFTSITDSLEQVLAKNNAITMNKRLYTEAWDNDKKTIHVMPDTPEIMLAKLNRINYSDKLYKVSLEEARRQGYDLRVDAIPIRSAKASREIASDYKYKEGYRKQLGHHIGARNIKDDPKMMWSMHVAKIQSDREYKKDFEKWKTKYSSPVDMLGVVLAKKCQILVSDIDYKHPLHEWTCLPDQNDVIQARKAYDLQSDAVYKADLEWLRGIGWVPIGSVEVEKVKRAGEILSDRKYRQPADQLKFTCITDTPEIVLAKSNAITMSKHLYTEAWDADKASIHVMPDTPEILLAKSNSANISHKLYTKGWDESKMKDYDLREDAISIKSAKASRDIASDYKYKEAYEKQKGHHIGAQSVEDDPRILCAMHAGKIYSDREYKKEFQKWKTKFSSPVDMLGVVLAKKCQTLVSDVDYRNYLHNWTCLPDQNDIIQAKKAYELQSDSVYKADLEWLRGIGWMPEGSIEMTRVKGAQDLVNERLYRTRPEALKFTSIVDTPEVVLAKANSLQMSERLYQEAWNKDKTNISLPLDTPAMLQAQINALQISNKLYQKDWDEAKQKGYDLRADAIEIKHAKASREIASEYKYKEGYRKQLGHHVGFRSLQDDPKLVWSIHAAKIQSDREYKKAYEKSKGIYNTPLDMMSIVQAKKCQVLVSDVDYRNYLHQWTCLPDQNDVIQAKKAYELQSDAIYKADLEWLRGIGWMPEGSPEVLRVKNAQEILCDSVYRTPVVKLKYTSIVDTPEVVLAKSNAENISIPKYREVWDKDKTSIHIMPDTPEINLARANALNVSNKLYREGWDEVKMSCDVRLDAIPIQAAKASREIASDYKYKLDHEKQKGHYVGTLTARDDNKIRWALIAGKIQNEREYRLQWAKWKTKFQSPVDMLSILHSKKCQTLVSDIDYRNYLHEWTCMPDQNDVIQAKKAYELQSDAVYKADLEWLRGIGWMPNDSVSVNHAKHAADIFSEKKYRTKIETLNFTPVDDRVDYVTAKQSSEIRDDIKYRKDWNAIKSKYTLTETPLLHTAQEAARILDQYLYKEGWEKQKATGYILPPDAVPFVHAHHSSDVQSELKYKAEHVKQRGHYVGVPTMRDDPKLVWFEHAGQIQNDRLYKESYHKSKAKINIPADMVSVVAAKEGQNLVSDIDYRQYLHQWTCHPDQNDVIQAKIAYDLQSDNVYKADLEWLRGIGWIPLDSVDHVRVTKNQEMVNQIKYKKDALANYPNFTSVEDPPEIVLAKINSVNQSDVKYKETFNKAKGKYIFSPDTPYISHSKDMGKLYSTILYKGAWEGTKAYGYTLDERYIPIVGAKHADLVNSELKYKETYEKQKGHYLAGKEISEFPGVVHCLDFQQMRSALNYRRDYEDTKANVHIPNDMMNHVLAKRCQYILSDLEYRHYFHQWTSLPEEPNVIRARNAQEILSDNVYKDDLNWLKGIGCYVWDTPQILHAKKSYDLQSQLQYTAASKENLQNYNLVTDTPVYVTALQSGINASEVKYKENYHQIKDKYTTVLETADYDRTKHLKSLYSSNIYKEAWDKVKATSYNLPTSTLSLTHAKNQKHLASHIKYREEYEKFKALYTLPKSVDDDPNTARCLRVGKLNIDRLYRSVYEKNKMKINIVPDMVEMVTAKDSQKKVSEIDYRLHLHEWICHPDLQVNSHVRKVTDQISDIVYKDDLTWLKGIGCYVWDTPEILHAKHAYDLRNDIKYKAHVQKTRNDYKLVTDTPVYVQAVKSGKQLSDAVYHYDYVHSVRGKVAPTTKTVDLDRALHAYKLQSENLYKKGLRFLPTGYRLPVDTPHFNHTKDTRYMSSYFKYKEAYEHIKANGYTLGPKDVPFVHVRRVNNVTSERLYRELYHKLKDKIHTTPDTPEIRQVKKTQEAVSELIYKSDFFKMQGHMISLPYTPQVLHCRYVGDITSDIKYKEDLQILKGLGCFLYDTPDMVRSRHLRKLWSNYLYTDNARKMRDKYKVVLDTPEYRKVQELKTHLSELVYRASGKKQKSIFTSVPDTPDLLRAKRGQKLQSQYLYVELATKERPHHHAGNQTTALQHAKDVKDMVSEKKYKIQYEKMKDKYTPVADTPILIRAKRAYWNASDLRYKETFQKTKGRYHTVKDALDIVYHRKVTDDISKVKYKENYMSQLGIWSSIPDRPEHFHHRAVTDAVSDVKYKEDLTWLKGIGCYAYDTPDLTLAEQNKTLYSKYKYKEVFERTKSNFKYVADCPINRHFKYATQLMNEKKYRADYEQRKDKYHLVVDEPRHLLAKTAGDQISQIKYRKKYEKSKDKFTSVVDTPEHLRTTKVNKQISDILYKLEYNKARPRGYTTIHDTPMLLHVRKVKDEVSDLKYKEVYQRNKSNCTIKPDAVHIKAAKDAYKVNTNLDYKKQYEANKAYWKWTPDRPDFIQAAKSSLQQSDFEYKLDREFLKGCKLSVTDDKDMVLALRNSLIESDLKYKEKHVKERGSCHAVPDTPQILLAKTVSNLVSENKYKDHVKKHLAQGSYTTLPETRDTVHVKAVTKHVSDTNYKKKFVKEKGKSNYSIMQEPPEVKHAMEVAKKQSDVAYKKDAKENLHYTTVADRPDIKKATQAAKQASEVEYRAKHRKEGSHGLSMLGRPDIEMAKKAAKLSSQVKYRENFDKEKGKTPKYNPKDSQLYKTMKDANNLASEVKYKADLKKLHKPVTDMKESLIMHHVLNTSQLASSYQYKKNYEKSKGHYHTIPDNLEQLHHREATELQSIVKYKEKYEKERGKPMLDFETPTYITAKESQQMQSGKEYRKDYEESIKGRNLTGLEVTPALLHVRHATKIASEKEYRKDLEESIRGRGLTEMEDTPDMLRAKNATQILNEKEYKRDLELEVKGRGLNAMANETPDFMRARNATDIASQIKYKQSAEMEKANFTSVVDTPEIIHAQQVKNLSSQKKYKEDAEKSMSYYETVLDTPEMQRVRENQKNFSLLQYQCDLKNSKGKITVVQDTPEILRVKENQKNFSSVLYKEDVSPGTAIGKTPEMLRVKQTQDHISSVKYKEAIGQGTPIPDLPEVKRVKETQKHISSVMYKENPGTGIPTTVTPEIERVKRNQENFSSVLYKESLGKGTPTAITPEMERVKRNQENFSSVLYKENMGKGTPLPVTPEMERVKHNQENISSVLYKENVGKATPTPVTPEMQRVKRNQENISSVLYKENMGKATPTPFTPEMERVKRNQENFSSVLYKENMRKATPTPVTPDMERAKRNQENISSVLYSDSFRKQIQGKAAYVLDTPEMRRVRETQRHISTVKYHEDFEKHKGCFTPVVTDPITERVKKNTQDFSDINYRGIQRKVVEMEQKRNDQDQDTITGLRVWRTNPGSVFDYDPAEDNIQSRSLHMINVQAQRRSRDQSRSASALSISGGEEKSEHSEAADQRLSTYSDGGAFFSATSTAYKHVKTTQLPQQRSSSVATQQTTLSSIPSHPSTAGKIFRAIYDYIAADADEVSFKDGDAIVNAQAIDEGWMYGTVQRTGRTGMLPANYVEAI

SEQ ID NO: 4 - protein, 377 aa, Mus musculus
MCDEDETTALVCDNGSGLVKAGFAGDDAPRAVFPSIVGRPRHQGVMVGMGQKDSYVGDEAQSKRGILTLKYPIEHGIITNWDDMEKIWHHTFYNELRVAPEEHPTLLTEAPLNPKANREKMTQIMFETFNVPAMYVAIQAVLSLYASGRTTGIVLDSGDGVTHNVPIYEGYALPHAIMRLDLAGRDLTDYLMKILTERGYSFVTTAEREIVRDIKEKLCYVALDFENEMATAASSSSLEKSYELPDGQVITIGNERFRCPETLFQPSFIGMESAGIHETTYNSIMKCDIDIRKDLYANNVMSGGTTMYPGIADRMQKEITALAPSTMKIKIIAPPERKYSVWIGGSILASLSTFQQMWITKQEYDEAGPSIVHRKCF

SEQ ID NO: 5 - protein, 381 aa, Mus musculus
MPFGNTHNKFKLNYKPQEEYPDLSKHNNHMAKVLTPDLYNKLRDKETPSGFTLDDVIQTGVDNPGHPFIMTVGCVAGDEESYTVFKDLFDPIIQDRHGGYKPTDKHKTDLNHENLKGGDDLDPNYVLSSRVRTGRSIKGYTLPPHCSRGERRAVEKLSVEALNSLTGEFKGKYYPLKSMTEQEQQQLIDDHFLFDKPVSPLLLASGMARDWPDARGIWHNDNKSFLVWVNEEDHLRVISMEKGGNMKEVFRRFCVGLQKIEEIFKKAGHPFMWNEHLGYVLTCPSNLGTGLRGGVHVKLANLSKHPKFEEILTRLRLQKRGTGGVDTAAVGAVFDISNADRLGSSEVEQVQLVVDGVKLMVEMEKKLEKGQSIDDMIPAQK

SEQ ID NO: 6 - protein, 900 aa, Mus musculus
MMMVMQPEGLGAGEGPFSGGGGGEYMEQEEDWDRDLLLDPAWEKQQRKTFTAWCNSHLRKAGTQIENIEEDFRNGLKLMLLLEVISGERLPRPDKGKMRFHKIANVNKALDFIASKGVKLVSIGAEEIVDGNLKMTLGMIWTIILRFAIQDISVEETSAKEGLLLWCQRKTAPYRNVNVQNFHTSWKDGLALCALIHRHRPDLIDYAKLRKDDPIGNLNTAFEVAEKYLDIPKMLDAEDIVNTPKPDEKAIMTYVSCFYHAFAGAEQAETAANRICKVLAVNQENEKLMEEYEKLASELLEWIRRTVPWLENRVGEPSMSAMQRKLEDFRDYRRLHKPPRVQEKCQLEINFNTLQTKLRLSHRPAFMPSEGKLVSDIANAWRGLEQVEKGYEDWLLSEIRRLQRLQHLAEKFQQKASLHEAWTRGKEEMLNQHDYESASLQEVRALLRRHEAFESDLAAHQDRVEHIAALAQELNELDYHEAASVNSRCQAICDQWDNLGTLTQKRRDALERMEKLLETIDQLQLEFARRAAPFNNWLDGAIEDLQDVWLVHSVEETQSLLTAHEQFKATLPEADRERGAILGIQGEIQKICQTYGLRPKSGNPYITLSSQDINNKWDTVRKLVPSRDQTLQEELARQQVNERLRRQFAAQANAIGPWIQGKVEEVGRLAAGLAGSLEEQMAGLRQQEQNIINYKSNIDRLEGDHQLLQESLVFDNKHTVYSMEHIRVGWEQLLTSIARTINEVENQVLTRDAKGLSQEQLNEFRASFNHFDRKRNGMMEPDDFRACLISMGYDLGEVEFARIMTMVDPNAAGVVTFQAFIDFMTRETAETDTAEQVVASFKILAGDKNYITPEELRRELPAEQAEYCIRRMAPYKGSGAPSGALDYVAFSSALYGESDL

SEQ ID NO: 7 - protein, 1942 aa, Mus musculus
MSSDAEMAVFGEAAPYLRKSEKERIEAQNKPFDAKSSVFVVDAKESFVKATVQSREGGKVTAKTEGGTTVTVKDDQVYPMNPPKYDKIEDMAMMTHLHEPAVLYNLKERYAAWMIYTYSGLFCVTVNPYKWLPVYNAEVVAAYRGKKRQEAPPHIFSISDNAYQFMLTDRENQSILITGESGAGKTVNTKRVIQYFATIAVTGEKKKEEATSGKMQGTLEDQIISANPLLEAFGNAKTVRNDNSSRFGKFIRIHFGTTGKLASADIETYLLEKSRVTFQLKAERSYHIFYQIMSNKKPDLIEMLLITTNPYDYAFVSQGEITVPSIDDQEELMATDSAIDILGFTSDERVSIYKLTGAVMHYGNMKFKQKQREEQAEPDGTEVADKAAYLQNLNSADLLKALCYPRVKVGNEYVTKGQTVQQVYNSVGALAKAVYEKMFLWMVTRINQQLDTKQPRQYFIGVLDIAGFEIFDFNSLEQLCINFTNEKLQQFFNHHMFVLEQEEYKKEGIEWEFIDFGMDLAACIELIEKPMGIFSILEEECMFPKATDTSFKNKLYEQHLGKSNNFQKPKPAKGKVEAHFSLVHYAGTVDYNIAGWLDKNKDPLNETVVGLYQKSSMKTLAYLFSGAAAAAEAESGGGGGKKGAKKKGSSFQTVSALFRENLNKLMTNLRSTHPHFVRCIIPNETKTPGAMEHELVLHQLRCNGVLEGIRICRKGFPSRILYADFKQRYKVLNASAIPEGQFIDSKKASEKLLGSIDIDHTQYKFGHTKVFFKAGLLGLLEEMRDDKLAQLITRTQAMCRGYLARVEYQKMVERRESIFCIQYNVRAFMNVKHWPWMKLYFKIKPLLKSAETEKEMANMKEEFEKAKENLAKAEAKRKELEEKMVALMQEKNDLQLQVQSEADSLADAEERCDQLIKTKIQLEAKIKEVTERAEDEEEINAELTAKKRKLEDECSELKKDIDDLELTLAKVEKEKHATENKVKNLTEEMAGLDETIAKLTKEKKALQEAHQQTLDDLQAEEDKVNTLTKAKIKLEQQVDDLEGSLEQEKKIRMDLERAKRKLEGDLKLAQESTMDVENDKQQLDEKLKKKEFEMSNLQSKIEDEQALGMQLQKKIKELQARIEELEEEIEAERASRAKAEKQRSDLSRELEEISERLEEAGGATSAQIEMNKKREAEFQKMRRDLEEATLQHEATAATLRKKHADSVAELGEQIDNLQRVKQKLEKEKSEMKMEIDDLASNMEVISKSKGNLEKMCRTLEDQVSELKTKEEEQQRLINELTAQRGRLQTESGEYSRQLDEKDSLVSQLSRGKQAFTQQIEELKRQLEEEIKAKSALAHALQSSRHDCDLLREQYEEEQEAKAELQRAMSKANSEVAQWRTKYETDAIQRTEELEEAKKKLAQRLQDAEEHVEAVNAKCASLEKTKQRLQNEVEDLMIDVERTNAACAALDKKQRNFDKILAEWKQKYEETHAELEASQKESRSLSTELFKIKNAYEESLDHLETLKRENKNLQQEISDLTEQIAEGGKRIHELEKIKKQIEQEKSELQAALEEAEASLEHEEGKILRIQLELNQVKSEIDRKIAEKDEEIDQLKRNHIRVVESMQSTLDAEIRSRNDAIRLKKKMEGDLNEMEIQLNHSNRMAAEALRNYRNTQGILKDTQLHLDDALRGQEDLKEQLAMVERRANLLQAEIEELRATLEQTERSRKIAEQELLDASERVQLLHTQNTSLINTKKKLETDISQIQGEMEDIVQEARNAEEKAKKAITDAAMMAEELKKEQDTSAHLERMKKNLEQTVKDLQHRLDEAEQLALKGGKKQIQKLEARVRELEGEVENEQKRNVEAIKGLRKHERRVKELTYQTEEDRKNVLRLQDLVDKLQSKVKAYKRQAEEAEEQSNVNLAKFRKIQHELEEAEERADIAESQVNKLRVKSREVHTKIISEE

SEQ ID NO: 8 - protein, 842 aa, Mus musculus [sequence omitted]

SEQ ID NO: 9 - protein, 364 aa, Mus musculus
MPHPYPALTPEQKKELSDIAHRIVAPGKGILAADESTGSIAKRLQSIGTENTEENRRFYRQLLLTADDRVNPCIGGVILFHETLYQKADDGRPFPQVIKSKGGVVGIKVDKGVVPLAGTNGETTTQGLDGLSERCAQYKKDGADFAKWRCVLKIGEHTPSALAIMENANVLARYASICQQNGIVPIVEPEILPDGDHDLKRCQYVTEKVLAAVYKALSDHHVYLEGTLLKPNMVTPGHACTQKFSNEEIAMATVTALRRTVPPAVTGVTFLSGGQSEEEASINLNAINKCPLLKPWALTFSYGRALQASALKAWGGKKENLKAAQEEYIKRALANSLACQGKYTPSGQSGAAASESLFISNHAY

SEQ ID NO: 10 - protein, 188 aa, Mus musculus
MAPKKDVKKPAAAPAPAPAPAPAPAKPKEEKIDLSAIKIEFSKEQQEDFKEAFLLFDRTGECKITLSQVGDVLRALGTNPTNAEVKKVLGNPSNEEMNAKKIEFEQFLPMMQAISNNKDQGGYEDFVEGLRVFDKEGNGTVMGAELRHVLATLGEKMKEEEVEALLAGQEDSNGCINYEAFVKHIMSV

SEQ ID NO: 11 - protein, 333 aa, Mus musculus
MVKVGVNGFGRIGRLVTRAAICSGKVEIVAINDPFIDLNYMVYMFQYDSTHGKFNGTVKAENGKLVINGKPITIFQERDPTNIKWGEAGAEYVVESTGVFTTMEKAGAHLKGGAKRVIISAPSADAPMFVMGVNHEKYDNSLKIVSNASCTTNCLAPLAKVIHDNFGIVEGLMTTVHAITATQKTVDGPSGKLWRDGRGAAQNIIPASTGAAKAVGKVIPELNGKLTGMAFRVPTPNVSVVDLTCRLEKPAKYDDIKKVVKQASEGPLKGILGYTEDQVVSCDFNSNSHSSTFDAGAGIALNDNFVKLISWYDNEYGYSNRVVDLMAYMASKE

SEQ ID NO: 12 - protein, 284 aa, Mus musculus
MDAIKKKMQMLKLDKENALDRAEQAEADKKAAEDRSKQLEDELVSLQKKLKGTEDELDKYSEALKDAQEKLELAEKKATDAEADVASLNRRIQLVEEELDRAQERLATALQKLEEAEKAADESERGMKVIESRAQKDEEKMEIQEIQLKEAKHIAEDADRKYEEVARKLVIIESDLERAEERAELSEGKCAELEEELKTVTNNLKSLEAQAEKYSQKEDKYEEEIKVLSDKLKEAETRAEFAERSVTKLEKSIDDLEDELYAQKLKYKAISEELDHALNDMTSI

SEQ ID NO: 13 - protein, 1136 aa, Mus musculus
MPEAKPAAKKASKGKDAPKEAPAKQTPEEPPKEAPPEDQSPTAEEPTGIFLKKPDSVSVETGKDAVILAKVNGKELPGKPTIKWFKGKWQELGSKSGARFIFKESHDSTSNVYTVELHIGKVVLGDRGDYRLEIKAKDVCDSCSFNVDVEAPRQDSSGQSLESFKRSGDGKSEDAGELDFSGLLKKREVVEEEKKKKKDDDDLGIPPEIWELLKGAKKSEYEKIAFQYGITDLRGMLKRLKKAKVEVKKSAAFTKKLDPAYQVDRGNKIKLVVEISDPDLPLKWFKNGQEIKPSSKYVFENVGKKRILTINKCTLADDAAYEVAVQDEKCFTELFVKEPPVLIVTPLEDQQVFVGDRVEMSVEVSEEGAQVMWMKDGVEMTREDSYKARYRFKKDGKRHILIYSDVAQEDGGRYQVITNGGQCEAELIVEEKQLEVLQDIADLTVKAAEQAVFKCEVSDEKVTGKWYKNGVEVRPSKRITISHVGRFHKLVIDDVRPEDEGDYTFVPDGYALSLSAKLNFLEIKVEYVPKQEPPKIHLDCSGKTSDNSIVVVAGNKLRLDVAITGEPPPTATWLRGDEVFTATEGRTHIEQRPDCSSFVIESAERSDEGRYTIKVTNPVGEDVASIFLRVVDVPDPPEAVRVTSVGEDWAILVWEPPKYDGGQPVTGYLMERKKKGSQRWMKINFEVFTDTTYESTKMIEGVLYEMRVFAVNAIGVSQPSMNTKPFMPIAPTSAPQHLTVEDVTDTTTTLKWRPPDRIGAGGIDGYLVEYCLEGSEEWVPANKEPVERCGFTVKDLPTGARILFRVVGVNIAGRSEPATLLQPVTIREIVEQPKIRLPRHLRQTYIRKVGEALNLVIPFQGKPRPQVVWTKGGAPLDTSRVNVRTSDFDTVFFVRQAARSDSGEYELSVQIENMKDTATIRIRVVEKAGPAENVMVKEVWGTNALVEWQPPKDDGNSEITGYFVQKADKKTMEWFNVYEHNRHTSCTVSDLIVGNEYYFRIFSENICGLSDSPGVSKNTARILKTGITLKPLEYKEHDFRTAPKFLTPLMDRVVVAGYTAALNCAVRGHPKPKVVWMKNKMEIHEDPKFLITNYQGILTLNIRRPSPFDAGTYSCRAFNELGEALAECKLDVRVPQ

SEQ ID NO: 14 - protein, 1270 aa, Mus musculus
MPEPGKKPVSAFNKKPRSAEVTAGSAAVFEAETERSGVMVRWQRDGSDITANDKYGLAAEGKRHTLTVRDASPDDQGSYAVIAGSSKVKFDLKVTEPAPPEKAESEVAPGAPEEVPAPATELEESVSSPEGSVSVTQDGSAAEHQGAPDDPIGLFLMRPQDGEVTVGGSIVFSARVAGASLLKPPVVKWFKGKWVDLSSKVGQHLQLHDSYDRASKVYLFELHITDAQTTSAGGYRCEVSTKDKFDSCNFNLTVHEAIGSGDLDLRSAFRRTSLAGAGRRTSDSHEDAGTPDFSSLLKKRDSFRRDSKLEAPAEEDVWEILRQAPPSEYERIAFQHGVEACHRPLKRLKGMKQDEKKSTAFQKKLEPAYQVNKGHKIRLTVELADPDAEVKWLKNGQEIQMSGSKYIFESVGAKRTLTISQCSLADDAAYQCVVGGEKCSTELFVKEPPVLITRSLEDQLVMVGQRVEFECEVSEEGAQVKWLKDGVELTREETFKYRFKKDGRKHHLIINEATLEDAGHYAVRTSGGQSLAELIVQEKKLEVYQSIADLAVGAKDQAVFKCEVSDENVRGVWLKNGKELVPDNRIKVSHIGRVHKLTIDDVTPADEADYSFVPEGFACNLSAKLHFMEVKIDFVPRQEPPKIHLDCPGSTPDTIVVVTGNKLRLDVPISGDPAPTVVWQKTVTQGKKASAGPHPDAPEDAGADEEWVFDKKLLCETEGRVRVETTKDRSVFTVEGAEKEDEGVYTVTVKNPVGEDQVNLTVKVIDVPDAPAAPKISNVGEDSCTVQWEPPAYDGGQPVLGYILERKKKKSYRWMRLNFDLLRELSHEARRMIEGVAYEMRVYAVNAVGMSRPSPASQPFMPIGPPGEPTHLAVEDVSDTTVSLKWRPPERVGAGGLDGYSVEYCQEGCSEWTPALQGLTERRSMLVKDLPTGARLLFRVRAHNVAGPGGPIVTKEPVTVQEILQRPRLQLPRHLRQTIQKKVGEPVNLLIPFQGKPRPQVTWTKEGQPLAGEEVSIRNSPTDTILFIRAARRTHSGTYQVTVRIENMEDKATLILQIVDKPSPPQDIRIVETWGFNVALEWKPPQDDGNTEIWGYTVQKADKKTMEWFTVLEHYRRTHCVVSELIIGNGYYFRVFSHNMVGSSDKAAATKEPVFIPRPGITYEPPKYKALDFSEAPSFTQPLANRSIIAGYNAILCCAVRGSPKPKISWFKNGLDLGEDARFRMFCKQGVLTLEIRKPCPYDGGVYVCRATNLQGEAQCECRLEVRVPQ

SEQ ID NO: 15 - protein, 1463 aa, Mus musculus
MSLVAVPFYQKRHKHFDQSYRNIQTRYLLDQYALKKQATTQSSSQRSLTERSSSKRASSQSSAGAMTCRLCAKRVSASEEEEVENENRYRSQAASYGEAKRQRFLSELAQLEENVHLARSQARDKLDKYFMEQTVEDNLAWERHSFEDRMSRAPEILVRLRSHTIWERMSVRLCFTVQGFPTPVVQWYKDGSLICQAGEPGKYRIESRYGVHTLEINRANFEDTATYSAVATNSHGQVSTNAAVVVRRYRGEEEPFHSVGLPIGLPLSSVIPYTHFDVQFLEKFGVTFRREGETVTLKCTLLVTPDLKRVQPRAEWYRDDVLLKESKWTKMFFGEGQASLSFSHLNKDDEGLYTLRIVSRGGVSDHSAFMFVRDADPLVTGAPGAPMDLQCHDANRDYVIVTWKPPNTTTESPVIGYFIDKCEVGTNNWVQCNDAPVKICKYPVTGLFEGRSYVFRVRAVNNAGISRPSRISDAVAALDPVDLRRLQAIHLEGEKEIVIYQDDLEGDVQIPGPPTNVQASEVSRNYVVLSWDPPSPRGKDPLMYFIEKSAVGSGSWQRVNAQTAVRSPRYAVFDLAEGKSYVFRVLSANKHGLSDPSEITPPIQAQDMIVVPSAPGRVLASRNTKTSVVVQWDRPKHEEDLLGYYVDCCVAGTNMWEPCNHKPIGYNRFVVHGLTTGEQYIFRVKAVNAVGTSENSQESEVIKVQAALTVPSHPYGITLLNCDGHSMTLGWKVPKFSGGSAIIGYYLDKREVHHKNWHEVNSSPVKERILTVEGLTEGSLYEFKIAATNLAGIGQPSDPSEHFKCEAWTDPEPGPAYDLTFCEVRDTSLVILWKAPVYSGSSPVSGYFVDFKEEDSGEWKTTSEAATPNRYLKVCDLQQGKTYVFRIRAVNASGPGKPSDTSEPVLVEARPGTKEISAGVDEEGNIYLGFDCQEMTDASQFTWCKAYEEIADEERFEVHTEGDHSKLYFKNPDKEDLGTYSVSVSDTDGVSSSFVLDEEELERLMALSNEIKNPTIPLKSELAYEIFDKGQVRFWLQAEHLSPDASFRFIINDREVSDSDTHRIKCDRSTGMIEMVMDRFTIENEGTYTVQIQDGKAKNQSSLVLIGDAFKAVLEEAEFQRKEFLRKQGPHFAEYLHWDVTEECEVRLVCKVANTKRETVFKWLKDDVLYETETPPDLEKGVCELLIPKLSKKDHGEYKATLKDDRGQDVSVLEVGGKVYEDMILAMSRVCGASASPLKVLCTPEGIRLQCFMKYFTEEMKVSWYHKEAKISSSEHMRIGGSEEMAWLQICEPTEKDKGKYTFEIFDGKDSHQRSLDLSGQAFDEAYAEFQQLKAAAFAEKNRGKVIGGLPDVVTIMEGKTLNLTCTVFGNPDPEVVWFKNDKDIELSEHFLVKMEQSKYVSLTIQGVTAEDSGKYSINVKNKYGGEKIDVTVSVYKHGEKIPDISPPQQAKPKLIPASTSSD

SEQ ID NO: 16 - protein, 608 aa, Mus musculus
MKWVTFLLLLFVSGSAFSRGVFRREAHKSEIAHRYNDLGEQHFKGLVLIAFSQYLQKCSYDEHAKLVQEVTDFAKTCVADESAANCDKSLHTLFGDKLCAIPNLRENYGELADCCTKQEPERNECFLQHKDDNPSLPPFERPEAEAMCTSFKENPTTFMGHYLHEVARRHPYFYAPELLYYAEQYNEILTQCCAEADKESCLTPKLDGVKEKALVSSVRQRMKCSSMQKFGERAFKAWAVARLSQTFPNADFAEITKLATDLTKVNKECCHGDLLECADDRAELAKYMCENQATISSKLQTCCDKPLLKKAHCLSEVEHDTMPADLPAIAADFVEDQEVCKNYAEAKDVFLGTFLYEYSRRHPDYSVSLLLRLAKKYEATLEKCCAEANPPACYGTVLAEFQPLVEEPKNLVKTNCDLYEKLGEYGFQNAILVRYTQKAPQVSTPTLVEAARNLGRVGTKCCTLPEDQRLPCVEDYLSAILNRVCLLHEKTPVSEHVTKCCSGSLVERRPCFSALTVDETYVPKEFKAETFTFHSDICTLPEKEKQIKKQTALAELVKHKPKATAEQLKTVMDDFAQFLDTCCKAADKDTCFSTEGPNLVTRCKDALA

SEQ ID NO: 17 - protein, 434 aa, Mus musculus
MAMQKIFAREILDSRGNPTVEVDLHTAKGRFRAAVPSGASTGIYEALELRDGDKARYLGKGVLKAVEHINKTLGPALLEKKLSVVDQEKVDKFMIELDGTENKSKFGANAILGVSLAVCKAGAAEKGVPLYRHIADLAGNPDLVLPVPAFNVINGGSHAGNKLAMQEFMILPVGASSFKEAMRIGAEVYHHLKGVIKAKYGKDATNVGDEGGFAPNILENNEALELLKTAIQAAGYPDKVVIGMDVAASEFYRNGKYDLDFKSPDDPARHISGEKLGELYKNFIQNYPVVSIEDPFDQDDWATWTSFLSGVDIQIVGDDLTVTNPKRIAQAVEKKACNCLLLKVNQIGSVTESIQACKLAQSNGWGVMVSHRSGETEDTFIADLVVGLCTGQIKTGAPCRSERLAKYNQLMRIEEALGDKAVFAGRKFRNPKAK

SEQ ID NO: 18 - protein, 169 aa, Mus musculus
MAPKKAKRRAGAEGSSNVFSMFDQTQIQEFKEAFTVIDQNRDGIIDKEDLRDTFAAMGRLNVKNEELDAMMKEASGPINFTVFLTMFGEKLKGADPEDVITGAFKVLDPEGKGTIKKQFLEELLTTQCDRFSQEEIKNMWAAFPPDVGGNVDYKNICYVITHGDAKDQE

SEQ ID NO: 19 - protein, 574 aa, Mus musculus
MSVQENELPQQLWPWIFKSQKDLAKSALSGAPGGPAGYLRRASVAQLTQELGTAFFQQQQLPAAMADTFLEHLCLLDIDSEPVAARSTSIIATIGPASRSVDRLKEMIKAGMNIARLNFSHGSHEYHAESIANIREAAESFATSPLSYRPVAIALDTKGPEIRTGVLQGGPESEVEIVKGSQVLVTVDPKFRTRGDAKTVWVDYHNITQVVAVGGRIYIDDGLISLVVRKIGPEGLVTEVEHGGFLGNRKGVNLPNAEVDLPGLSEQDLLDLRFGVEHYVDIIFASFVRKASDVVAVRDALGPEGRGIKIISKIENHEGVKKFDEILEVSDGIMMARGDLGIEIPAEKVFLAQKMMIGRCNLAGKPVVCATQMLESMITKARPTRAETSDVANAVLDGADCIMLSGETAKGSFPVEAVKMQHAIAREAEAAVYHRQLFEELRRAAPLSRDPTEVTAIGAVEASFKCCAAAIIVLTKTGRSAQLLSRYRPRAAVIAVTRSAQAARQVHLSRGVFPLLYREPPEAVWADDVDRRVQFGIESGKLRGFLRVGDLVIVVTGWRPGSGYTNIMRVLTIS

SEQ ID NO: 20 - protein, 260 aa, Mus musculus
MAKEWGYASHNGPDHWHELYPIAKGDNQSPIELHTKDIKHDPSLQPWSASYDPGSAKTILNNGKTCRVVFDDTYDRSMLRGGPLSGPYRLRQFHLHWGSSDDHGSEHTVDGVKYAAELHLVHWNPKYNTFGEALKQPDGIAVVGIFLKIGREKGEFQILLDALDKIKTKGKEAPFTHFDPSCLFPACRDYWTYHGSFTTPPCEECIVWLLLKEPMTVSSDQMAKLRSLFSSAENEPPVPLVGNWRPPQPVKGRVVRASFK

SEQ ID NO: 21 - protein, 332 aa, Mus musculus
MATLKDQLIVNLLKEEQAPQNKITVVGVGAVGMACAISILMKDLADELALVDVMEDKLKGEMMDLQHGSLFLKTPKIVSSKDYCVTANSKLVIITAGARQQEGESRLNLVQRNVNIFKFIIPNIVKYSPHCKLLIVSNPVDILTYVAWKISGFPKNRVIGSGCNLDSARFRYLMGERLGVHALSCHGWVLGEHGDSSVPVWSGVNVAGVSLKSLNPELGTDADKEQWKEVHKQVVDSAYEVIKLKGYTSWAIGLSVADLAESIMKNLRRVHPISTMIKGLYGINEDVFLSVPCILGQNGISDVVKVTLTPEEEARLKKSADTLWGIQKELQF

SEQ ID NO: 22 - protein, 1942 aa, Mus musculus
MSSDAEMAVFGEAAPYLRKSEKERIEAQNRPFDAKTSVFVAEPKESFVKGTIQSKDAGKVTVKTEAGATLTVKEDQIFPMNPPKYDKIEDMAMMTHLHEPAVLYNLKERYAAWMIYTYSGLFCVTVNPYKWLPVYNPEVVAAYRGKKRQEAPPHIFSISDNAYQFMLTDRENQSILITGESGAGKTVNTKRVIQYFATIAVTGDKKKEEATSGKMQGTLEDQIISANPLLEAFGNAKTVRNDNSSRFGKFIRIHFGTTGKLASADIETYLLEKSRVTFQLKAERSYHIFYQITSNKKPELIEMLLITTNPYDYPFVSQGEISVASIDDQEELMATDSAIDILGFTNDEKVSIYKLTGAVMHYGNMKFKQKQREEQAEPDGTEVADKAAYLQGLNSADLLKALCYPRVKVGNEYVTKGQTVEQVTNAVGALAKAMYEKMFLWMVTRINQQLDTKQPRQYFIGVLDIAGFEIFDFNSLEQLCINFTNEKLQQFFNHHMFVLEQEEYKKEGIEWTFIDFGMDLAACIELIEKPMGIFSILEEECMFPKATDTSFKNKLYEQHLGKSANFQKPKVVKGKAEAHFSLIHYAGTVDYNITGWLDKNKDPLNETVVGLYQKSSVKTLAYLFSGAQTAEAEASSGGAAKKGAKKKGSSFQTVSALFRENLNKLMTNLRSTHPHFVRCIIPNETKTPGAMEHELVLHQLRCNGVLEGIRICRKGFPSRILYADFKQRYKVLNASAIPEGQYIDSKKASEKLLGSIDIDHTQYKFGHTKVFFKAGLLGLLEEMRDDKLAQLITRTQAMCRGFLARVEYQKMVERRESIFCIQYNIRAFMNVKHWPWMKLFFKIKPLLKSAETEKEMATMKEEFQKTKDDLAKSEAKRKELEEKMVSLLKEKNDLQLQVQAEAEGLADAEERCDQLIKTKIQLEAKIKEVTERAEDEEEINAELTAKKRKLEDECSELKKDIDDLELTLAKVEKEKHATENKVKNLTEEMAGLDETIAKLTKEKKALQEAHQQTLDDLQAEEDKVNTLTKAKIKLEQQVDDLEGSLEQEKKLRMDLERAKRKLEGDLKLAQESIMDIENEKQQLDERLKKKEFEMSNLQSKIEDEQAIGIQLQKKIKELQARIEELEEEIEAERASRAKAEKQRSDLSRELEEISERLEEAGGATSAQIEMNKKREAEFQKMRRDLEEATLQHEATAATLRKKHADSVAELGEQIDNLQRVKQKLEKEKSEMKMEIDDLASNVETVSKAKGNLEKMCRTLEDQVSELKSKEEEQQRLINDLTTQRGRLQTESGEFSRQLDEKEALVSQLSRGKQAFTQQIEELKRQLEEEVKAKNALAHALQSSRHDCDLLREQYEEEQESKAELQRALSKANSEVAQWRTKYETDAIQRTEELEEAKKKLAQRLQAAEEHVEAVNAKCASLEKTKQRLQNEVEDLMLDVERTNAACAALDKKQRNFDKILAEWKQKYEETHAELEASQKEARSLGTELFKMKNAYEESLDQLETLKRENKNLQQEISDLTEQIAEGGKRIHELEKIKKQVEQEKCELQAALEEAEASLEHEEGKILRIQLELNQVKSEIDRKIAEKDEEIDQLKRNHIRVVESMQSTLDAEIRSRNDAIRIKKKMEGDLNEMEIQLNHSNRMAAEALRNYRNTQGILKDTQLHLDDALRGQEDLKEQLAMVERRANLLQAEIEELRATLEQTERSRKIAEQELLDASERVQLLHTQNTSLINTKKKLETDISQIQGEMEDIVQEARNAEEKAKKAITDAAMMAEELKKEQDTSAHLERMKKNMEQTVKDLQLRLDEAEQLALKGGKKQIQKLEARVRELEGEVESEQKRNAEAVKGLRKHERRVKELTYQTEEDRKNILRLQDLVDKLQAKVKSYKRQAEEAEEQSNTNLSKFRKIQHELEEAEERADIAESQVNKLRVKSREVHTKIISEE

SEQ ID NO: 23 - protein, 1124 aa, Mus musculus
MPEPTKKEENEVSAPAPPPEEPNKEKEAGTAPAKEWSLGESPAGGEEQDKQNANSQLSTLFVEKPQTGSVKVGANITFVAKVRAEDLLRKPTVKWFKGKWMDLASKAGKHLQLKETFERQTRVYTFEMQIIKAKENYAGNYRCEVTYKDKFDSCSFDLEVHESTGTTPNIDIRSAFKRSGEGQEDAGELDFSGLLKRREVKQQEEEPEIDVWELLKNANPNEYEKIAFQYGITDLRGMLKRLKRMRRVEKKSAAFAKILDPAYQVDKGGKVRFVVELADPKLEVKWFKNGQEIRPSTKYIFEHKGNERIMFINNCSLTDDSEYYVTAGDEKCSTELFVREPPIMVTKQLEDMNAYCGERVEMEVEVSEDDANVKWFKNGEEIFPGPKSRYKIKVEGKKHTLIIEGATKADSAEYSAMTTGGQSSAKLSVDLRPLKITTPLTDQTVKLGKEVCLKCEISENVPGKWTKNGLPVQEGERLKVVHKGRIHKLVIANALIEDEGEYVFTPDAYNVPLSAKVHVIDPPKIILDGLDADNTVTVIAGSKLRLEIPVTGEPPPKAIWSRADKAIMEGSGRIRAESYPDSSTLVIDVAERDDSGVYNINLKNEAGEAHASIKIKVVDIPDPPVAPNVTEVGDDWCIMNWEPPAYDGGSPILGYFIERKKKQSSRWMRLNFDLCKETTFEPKKMIEGVAYEVRIFAVNAIGISKPSMPSKPFVPLAVTSPPTLLAVDSVTDSSVTMKWRPPDQIGAAGLDGYVLEYCFEGTEDWIVANTDLIDKTKFTINGLPTDAKIFVRVKAINAAGASEPKYYSQPILVKEIIEPPKIRIPRHLKQTYIRRVGEAVNLVIPFQGKPRPELTWKKDGEEIDKNQINIRNSETDTIIFIRKAERSHSGKYDLQVKVDKYVENASIDIQIVDRPGPPQTVTIEDVWGENVALTWTPPKDDGNAAITGYTIQKADKKSMEWFTVIEHYHRTNATITELVIGNEYYFRVFAENMCGLSEDATMTKESAVIAKDGKIYKNPVYEDFNFTEAPMFTQPLVNTYAIAGYNATLNCSVRGNPKPKITWMKNKVAIVDDPRYRMFSNQGVCTLEIRKPSPYDGGTYCCKAVNDLGTVEIECKLEVKVVAQ

SEQ ID NO: 24 - protein, 562 aa, Mus musculus
MVKIVTVKTQAYPDQKPGTSGLRKRVKVFQSNANYAENFIQSIVSTVEPALRQEATLVVGGDGRFYMTEAIQLIVRIAAANGIGRLVIGQNGILSTPAVSCIIRKIKAIGGIILTASHNPGGPNGDFGIKFNISNGGPAPEAITDKIFQISKTIEEYAICPDLKVDLGVLGKQQFDLENKFKPFTVEIVDSVEAYATMLRNIFDFNALKELLSGPNRLKIRIDAMHGVVGPYVKKILCEELGAPANSAVNCVPLEDFGGHHPDPNLTYAADLVETMKSGEHDFGAAFDGDGDRNMILGKHGFFVNPSDSVAVIAANIFSIPYFQQTGVRGFARSMPTSGALDRVANATKIALYETPTGWKFFGNLMDASKLSLCGEESFGTGSDHIREKDGLWAVLAWLSILATRKQSVEDILKDHWQKFGRNFFTRYDYEEVEAEGANKMMKDLEALMLDRSFVGKQFSANDKVYTVEKADNFEYSDPVDGSISKNQGLRLIFADGSRIIFRLSGTGSAGATIRLYIDSYEKDVAKINQDPQVMLAPLISIALKVSQLQERTGRTAPTVIT

SEQ ID NO: 25 - protein, 284 aa, Mus musculus
MDAIKKKMQMLKLDKENAIDRAEQAEADKKQAEDRCKQLEEEQQALQKKLKGTEDEVEKYSESVKDAQEKLEQAEKKATDAEADVASLNRRIQLVEEELDRAQERLATALQKLEEAEKAADESERGMKVIENRAMKDEEKMELQEMQLKEAKHIAEDSDRKYEEVARKLVILEGELERSEERAEVAESKCGDLEEELKIVTNNLKSLEAQADKYSTKEDKYEEEIKLLEEKLKEAETRAEFAERSVAKLEKTIDDLEDEVYAQKMKYKAISEELDNALNDITSL

SEQ ID NO: 26 - protein, 249 aa, Mus musculus
MAPTRKFFVGGNWKMNGRKKCLGELICTLNAANVPAGTEVVCAPPTAYIDFARQKLDPKIAVAAQNCYKVTNGAFTGEISPGMIKDLGATWVVLGHSERRHVFGESDELIGQKVSHALAEGLGVIACIGEKLDEREAGITEKVVFEQTKVIADNVKDWSKVVLAYEPVWAIGTGKTATPQQAQEVHEKLRGWLKSNVNDGVAQSTRIIYGGSVTGATCKELASQPDVDGFLVGGASLKPEFVDIINAKQ

SEQ ID NO: 27 - protein, 253 aa, Mus musculus
MTTHRLVMVRHGESLWNQENRFCGWFDAELSEKGAEEAKRGATAIKDAKIEFDICYTSVLKRAIRTLWTILDVTDQMWVPVVRTWRLNERHYGGLTGLNKAETAAKHGEEQVKIWRRSFDTPPPPMDEKHNYYTSISKDRRYAGLKPEELPTCESLKDTIARALPFWNEEIAPKIKAGQRVLIAAHGNSLRGIVKHLEGMSDQAIMELNLPTGIPIVYELDQNLKPTKPMRFLGDEETVRKAMEAVAAQGKAK

SEQ ID NO: 28 - protein, 472 aa, Mus musculus
MKALLLLCCFLASLLLSGQAEVEDASEEAPLRDRSHIDKTLMLNEDKPADDYSAVLQRLRKIYHTSIKPLEQSYKYNELRQHEITDGEITSKPMVLFLGPWSVGKSTMINYLLGLEDTRYQLYTGAEPTTSEFTVLMHGPKLKTIEGIVMAADSARSFSPLEKFGQNFLEKLIGIEVPHKLLERVTFVDTPGIIENRKQQERGYPFNDVCQWFIDRADLIFVVFDPTKLDVGLELEMLFRQLKGRESQIRIILNKADNLATQMLMRVYGALFWSLAPLINVTEPPRVYVSSFWPQDYKPDTHRELFLKEEISLLEDLNQVIENRLENKIAFIRQHAIRVRIHALLVDRYLQTYKDKMTFFSDGELVFKDIVEDPDKFYIFKTILAKTNVSKFDLPNREAYKDFFGINPISNFKLLSQQCSYMGGCFLEKIERAITQELPSLLGSIGLGKNPGAPNCDKTGCGETPKNRYKKH

SEQ ID NO: 29 - protein, 417 aa, Mus musculus
MSLSNKLTLDKLDVKGKRVVMRVDFNVPMKNNQITNNQRIKAAVPSIKFCLDNGAKSVVLMSHLGRPDGVPMPDKYSLEPVAAELKSLLGKDVLFLKDCVGPEVENACANPAAGTVILLENLRFHVEEEGKGKDASGNKVKAEPAKIDAFRASLSKLGDVYVNDAFGTAHRAHSSMVGVNLPQKAGGFLMKKELNYFAKALESPERPFLAILGGAKVADKIQLINNMLDKVNEMIIGGGMAFTFLKVLNNMEIGTSLYDEEGAKIVKDLMSKAEKNGVKITLPVDFVTADKFDENAKTGQATVASGIPAGWMGLDCGTESSKKYAEAVGRAKQIVWNGPVGVFEWEAFARGTKSLMDEVVKATSRGCITIIGGGDTATCCAKWNTEDKVSHVSTGGGASLELLEGKVLPGVDALSNV

SEQ ID NO: 30 - protein, 4691 aa, Mus musculus
MVAGMLMPLDRLRAIYEVLFREGVMVAKKDRRPRSLHPHVPGVTNLQVMRAMASLKARGLVRETFAWCHFYWYLTNEGIDHLRQYLHLPPEIVPASLQRVRRPVAMVIPARRRSPHVQTMQGPLGCPPKRGPLPAEDPAREERQVYRRKEREEGAPETPVVSATTVGTLARPGPEPAPATDERDRVQKKTFTKWVNKHLIKHWRAEAQRHISDLYEDLRDGHNLISLLEVLSGDSLPREKGRMRFHKLQNVQIALDYLRHRQVKLVNIRNDDIADGNPKLTLGLIWTIILHFQISDIQVSGQSEDMTAKEKLLLWSQRMVEGYQGLRCDNFTTSWRDGRLFNAIIHRHKPMLIDMNKVYRQTNLENLDQAFSVAERDLGVTRLLDPEDVDVPQPDEKSIITYVSSLYDAMPRVPGAQDGVRANELQLRWQEYRELVLLLLQWIRHHTAAFEERKFPSSFEEIEILWCQFLKFKETELPAKEADKNRSKVIYQSLEGAVQAGQLKIPPGYHPLDVEKEWGKLHVAILEREKQLRSEFERLECLQRIVSKLQMEAGLCEEQLNQADALLQSDIRLLASGKVAQRAGEVERDLDKADGMIRLLFNDVQTLKDGRHPQGEQMYRRVYRLHERLVAIRTEYNLRLKAGVGAPVTQVTLQSTQRRPELEDSTLRYLQDLLAWVEENQRRIDSAEWGVDLPSVEAQLGSHRGMHQSIEEFRAKIERARNDESQLSPATRGAYRDCLGRLDLQYAKLLNSSKARLRSLESLHGFVAAATKELMWLNEKEEEEVGFDWSDRNTNMAAKKESYSALMRELEMKEKKIKEIQNTGDRLLREDHPARPTVESFQAALQTQWSWMLQLCCCIEAHLKENTAYFQFFSDVREAEEQLQKLQETLRRKYSCDRTITVTRLEDLLQDAQDEKEQLNEYKGHLSGLAKRAKAIVQLKPRNPAHPVRGHVPLIAVCDYKQVEVTVHKGDQCQLVGPAQPSHWKVLSGSSSEAAVPSVCFLVPPPNQEAQEAVARLEAQHQALVTLWHQLHVDMKSLLAWQSLSRDIQLIRSWSLVTFRTLKPEEQRQALRNLELHYQAFLRDSQDAGGFGPEDRLVAEREYGSCSRHYQQLLQSLEQGEQEESRCQRCISELKDIRLQLEACETRTVHRLRLPLDKDPARECAQRIAEQQKAQAEVEGLGKGVARLSAEAEKVLALPEPSPAAPTLRSELELTLGKLEQVRSLSAIYLEKLKTISLVIRSTQGAEEVLKTHEEQLKEAQAVPATLQELEATKASLKKLRAQAEAQQPVFNTLRDELRGAQEVGERLQQRHGERDVEVERWRERVTQLLERWQAVLAQTDVRQRELEQLGRQLRYYRESADPLSAWLQDAKRRQEQIQAVPIANCQAAREQLRQEKALLEEIERHGEKVEECQKFAKQYINAIKDYELQLITYKAQLEPVASPAKKPKVQSGSESVIQEYVDLRTRYSELTTLTSQYIKFISETLRRMEEEERLAEQQRAEERERLAEVEAALEKQRQLAEAHAQAKAQAELEAQELQRRMQEEVARREEAAVDAQQQKRSIQEELQHLRQSSEAEIQAKAQQVEAAERSRMRIEEEIRVVRLQLETTERQRGGAEGELQALRARAEEAEAQKRQAQEEAERLRRQVQDESQRKRQAEAELALRVKAEAEAAREKQRALQALDELRLQAEEAERRLRQAEAERARQVQVALETAQRSAEVELQSKRASFAEKTAQLERTLQEEHVTVAQLREEAERRAQQQAEAERAREEAERELERWQLKANEALRLRLQAEEVAQQKSLAQADAEKQKEEAEREARRRGKAEEQAVRQRELAEQELEKQRQLAEGTAQQRLAAEQELIRLRAETEQGEQQRQLLEEELARLQHEATAATQKRQELEAELAKVRAEMEVLLASKARAEEESRSTSEKSKQRLEAEAGRFRELAEEAARLRALAEEAKRQRQLAEEDAARQRAEAERVLTEKLAAISEATRLKTEAEIALKEKEAENERLRRLAEDEAFQRRRLEEQAALHKADIEERLAQLRKASESELERQKGLVEDTLRQRRQVEEEIMALKVSFEKAAAGKAELELELGRIRSNAEDTMRSKEQAELEAARQRQLAAEEEQRRREAEERVQRSLAAEEEAARQRKVALEEVERLKAKVEEARRLRERAEQESARQLQLAQEAAQKRLQAEEKAHAFVVQQREEELQQTLQQEQNMLDRLRSEAEAARRAAEEAEEAREQAEREAAQSRKQVEEAERLKQSAEEQAQAQAQAQAAAEKLRKEAEQEAARRAQAEQAALKQKQAADAEMEKHKKFAEQTLRQKAQVEQELTTLRLQLEETDHQKSILDEELQRLKAEVTEAARQRSQVEEELFSVRVQMEELGKLKARIEAENRALILRDKDNTQRFLEEEAEKMKQVAEEAARLSVAAQEAARLRQLAEEDLAQQRALAEKMLKEKMQAVQEATRLKAEAELLQQQKELAQEQARRLQEDKEQMAQQLVEETQGFQRTLEAERQRQLEMSAEAERLKLRMAEMSRAQARAEEDAQRFRKQAEEIGEKLHRTELATQEKVTLVQTLEIQRQQSDHDAERLREAIAELEREKEKLKQEAKLLQLKSEEMQTVQQEQILQETQALQKSFLSEKDSLLQRERFIEQEKAKLEQLFQDEVAKAKQLREEQQRQQQQMEQEKQELMASMEEARRRQREAEEGVRRKQEELQHLEQQRQQQEKLLAEENQRLRERLQRLEEEHRAALAHSEIATTQAASTKALPNGRDAPDGPSVEAEPEYTFEGLRQKVPAQQLQEAGILSQEELQRLAQGHTTVAELTQREDVYRYLKGRSSIAGLLLKPTNEKLSVYTALQRQLLSPGTALILLEAQAASGFLLDPVRNRRLTVNEAVKEGVVGPELHHKLLSAERAVTGYKDPYTGEQISLFQAMKKDLIVRDHGVRLLEAQIATGGIIDPVHSHRVPVDVAYKRGYFDEEMNRILSDPSDDTKGFFDPNTHENLTYLQLLERCVEDPETGLRLLPLTDKAAKGGELVYTDTEARDVFEKATVSAPFGKFQGRTVTIWEIINSEYFTAEQRRDLLQQFRTGHITVEKIIKIVITVVEEHERKGQLCFEGLRALVPAAELLDSGVISHELYQQLQRGERSVREVAEADSVRQALRGTNVIAGVWLEEAGQKLSIYEALKKDLLQPEVAVALLEAQAGTGHIIDPATSARLTVDEAVRAGLVGPELHEKLLSAEKAVTGYRDPYSGQSVSLFQALKKGLIPREQGLRLLDAQLSTGGIVDPSKSHRVPLDVAYARGYLDKETNRALTSPRDDARVYHDPSTQEPVTYSQLQQRCRSDQLTGLSLLPLSEKAVRARQEEVYSELQARETLEQAKVEVPVGSFKGRAMTVWELISSEYFTEEQRQELLRQFRTGKVTVEKVIKIVITIVEEVETRRQERLSFSGLRAPVPASELLDAKILSRAQFDQLKDGKTSVKELSEVGSVRTLLQGSGCLAGIYLEDSKEKVTIYEAMRRGLLRPSTATLLLEAQAATGFLVDPVRNQRLYVHEAVKAGVVGPELHEKLLSAEKAVTGYKDPYSGNTISLFQAMKKGLVLRDHAIRLLEAQVATGGIIDPVHSHRLPVDVAYQRGYFDEEMNRVLADPSDDTKGFFDPNTHENLTYLQLLERCVEDPETGLRLLPLKGAEKTEVVETTQVYTEEETRRAFEETQIDIPGGGSHGGSSMSLWEVMQSNMIPEDQRARLMADFQAGRVTKERMIIIIIEIIEKTEIIRQQNLASYDYVRRRLTAEDLYEARIISLETYNLFREGTKNLREVLEMESAWRYLYGTGAVAGVYLPGSRQTLTIYQALKKGLLSAEVARLLLEAQAATGFLLDPVKGERLTVDEAVRKGLVGPELHDRLLSAERAVTGYRDPYTEQTISLFQAMKKELIPAEEALRLLDAQLATGGIVDPRLGFHLPLEVAYQRGYLNKDTHDQLSEPSEVRSYVDPSTDERLSYTQLLKRCRRDDPSGQMLLLLSDARKLTFRGLRKQITVEELVRSQVMDEATALQLQEGLTSIEEVTKNLQKFLEGTSCIAGVFVDATKERLSVYQAMKKGIIRPGTAFELLEAQAATGYVIDPIKGLKLTVEEAVRMGIVGPEFKDKLLSAERAVTGYKDPYSGKLISLFQAMKKGLILKDHGIRLLEAQIATGGIIDPEESHRLPVEVAYKRGLFDEEMNEILTDPSDDTKGFFDPNTEENLTYLQLMERCITDPQTGLCLLPLKEKKRERKTSSKSSVRKRRVVIVDPETGKEMSVYEAYRKGLIDHQTYLELSEQECEWEEITISSSDGVVKSMIIDRRSGRQYDIDDAITKNLIDRSALDQYRAGTLSITEFADMLSGNAGGFRSRSSSVGSSSSYPISSAGPRTQLASWSDPTEETGPVAGILDTETLEKVSITEAMHRNLVDNITGQRLLEAQACTGGIIDPSTGERFPVTEAVNKGLVDKIMVDRINLAQKAFCGFEDPRTKTKMSAAQALKKGWLYYEAGQRFLEVQYLTGGLIEPDTPGRVSLDEALQRGTVDARTAQKLRDVSAYSKYLTCPKTKLKISYKDALDRSMVEEGTGLRLLEAAAQSSKGYYSPYSVSGSGSTAGSRTGSRTGSRAGSRRGSFDATGSGFSMTFSSSSYSSSGYGRRYASGPSASLGGPESAVA

SEQ ID NO: 31 - protein, 894 aa, Mus musculus
MNQIEPGVQYNYVYDEDEYMIQEEEWDRDLLLDPAWEKQQRKTFTAWCNSHLRKAGTQIENIEEDFRNGLKLMLLLEVISGERLPKPDRGKMRFHKIANVNKALDYIASKGVKLVSIGAEEIVDGNVKMTLGMIWTIILRFAIQDISVEETSAKEGLLLWCQRKTAPYRNVNIQNFHTSWKDGLGLCALIHRHRPDLIDYSKLNKDDPIGNINLAMEIAEKHLDIPKMLDAEDIVNTPKPDERAIMTYVSCFYHAFAGAEQAETAANRICKVLAVNQENERLMEEYERLASELLEWIRRTIPWLENRTPEKTMQAMQKKLEDFRDYRRKHKPPKVQEKCQLEINFNTLQTKLRISNRPAFMPSEGKMVSDIAGAWQRLEQAEKGYEEWLLNEIRRLERLEHLAEKFRQKASTHETWAYGKEQILLQKDYESASLTEVRALLRKHEAFESDLAAHQDRVEQIAAIAQELNELDYHDAVNVNDRCQKICDQWDRLGTLTQKRREALERTEKLLETIDQLHLEFAKRAAPFNNWMEGAMEDLQDMFIVHSIEEIQSLITAHEQFKATLPEADGERQSILAIQNEVEKVIQSYSIRISSSNPYSTVTMDELRNKWDKVKQLVPVRDQSLQEELARQHANERLRRQFAAQANAIGPWIQNKMEEIARSSIQITGALEDQMNQLKQYEHNIINYKNNIDKLEGDHQLIQEALVFDNKHTNYTMEHIRVGWELLLTTIARTINEVETQILTRDAKGITQEQMNEFRASFNHFDRRKNGLMDHEDFRACLISMGYDLGEAEFARIMTLVDPNGQGTVTFQSFIDFMTRETADTDTAEQVIASFRILASDKPYILAEELRRELPPDQAQYCIKRMPPYSGPGSVPGALDYTAFSSALYGESDL

SEQ ID NO: 32 - protein, 780 aa, Mus musculus
MTHEEHHAAKTLGIGKAIAVLTSGGDAQGMNAAVRAVVRVGIFTGARVFFVHEGYQGLVDGGEHIREATWESVSMMLQLGGTVIGSARCKDFREREGRLRAAHNLVKRGITNLCVIGGDGSLTGADTFRSEWSDLLNDLQKDGKITAEEATKSSYLNIVGLVGSIDNDFCGTDMTIGTDSALHRIVEIVDAITTTAQSHQRTFVLEVMGRHCGYLALVTSLSCGADWVFIPECPPDDDWEEHLCRRLSETRTRGSRLNIIIVAEGAIDKNGKPITSEDIKNLVVKRLGYDTRVTVLGHVQRGGTPSAFDRILGSRMGVEAVMALLEGTPDTPACVVSLSGNQAVRLPLMECVQVTKDVTKAMDEKRFDEAIKLRGRSFMNNWEVYKLLAHVRPPVSKGGLHTVAVMNVGAPAAGMNAAVRSTVRIGLIQGNRVLVVHDGFEGLAKGQIEEAGWSYVGGWTGQGGSKLGTKRTLPKKNLEQISANITKFNIQGLVIIGGFEAYTGGLELMEGRKQFDELCIPFVVIPATVSNNVPGSDFSIGADTALNTICTTCDRIKQSAAGTKRRVFIIETMGGYCGYLATMAGLAAGADAAYIFEEPFTIRDLQVNVEHLVQKMKTTVKRGLVLRNEKCNENYTTDFIFNLYSEEGKGIFDSRKNVLGHMQQGGSPTPFDRNFATKMGAKAMNWMSGKIKESYRNGRIFANTPDSGCVLGMRKRALVFQPVTELKDQTDFEHRIPKEQWWLKLRPILKILAKYEIDLDTSDHAHLEHISRKRSGEAAV

SEQ ID NO: 33 - protein, 338 aa, Mus musculus
MLSALARPAGAALRRSFSTSAQNNAKVAVLGASGGIGQPLSLLLKNSPLVSRLTLYDIAHTPGVAADLSHIETRANVKGYLGPEQLPDCLKGCDVVVIPAGVPRKPGMTRDDLFNTNATIVATLTAACAQHCPEAMVCIIANPVNSTIPITAEVFKKHGVYNPNKIFGVTTLDIVRANTFVAELKGLDPARVNVPVIGGHAGKTIIPLISQCTPKVDFPQDQLATLTGRIQEAGTEVVKAKAGAGSATLSMAYAGARFVFSLVDAMNGKEGVVECSFVQSKETECTYFSTPLLLGKKGLEKNLGIGKITPFEEKMIAEAIPELKASIKKGEDFVKNMK

SEQ ID NO: 34 - protein, 154 aa, Mus musculus
MGLSDGEWQLVLNVWGKVEADLAGHGQEVLIGLFKTHPETLDKFDKFKNLKSEEDMKGSEDLKKHGCTVLTALGTILKKKGQHAAEIQPLAQSHATKHKIPVKYLEFISEIIIEVLKKRHSGDFGADAQGAMSKALELFRNDIAAKYKELGFQG

SEQ ID NO: 35 - protein, 147 aa, Mus musculus
MVHLTDAEKAAVSCLWGKVNSDEVGGEALGRLLVVYPWTQRYFDSFGDLSSASAIMGNAKVKAHGKKVITAFNDGLNHLDSLKGTFASLSELHCDKLHVDPENFRLLGNMIVIVLGHHLGKDFTPAAQAAFQKVVAGVATALAHKYH

SEQ ID NO: 36 - protein, 2726 aa, Mus musculus
MMNNSNYSDASGLGLVDEADEMPSTEKDLAEDAPWKKIQQNTFTRWCNEHLKCVGKRLTDLQRDLSDGLRLIALLEVLSQKRMYRKFHPRPNFRQMKLENVSVALEFLEREHIKLVSIDSKAIVDGNLKLILGLIWTLILHYSISMPMWEDEDDEDARKQTPKQRLLGWIQNKVPQLPITNFNRDWQDGKALGALVDNCAPGLCPDWEAWDPNQPVQNAREAMQQADDWLGVPQVIAPEEIVDPNVDEHSVMTYLSQFPKAKLKPGAPVRSKQLNPKKAIAYGPGIEPQGNTVLQPAHFTVQTVDAGVGEVLVYIEDPEGHTEEAKVVPNNDKDRTYAVSYVPKVAGLHKVTVLFAGQNIERSPFEVNVGMALGDANKVSARGPGLEPVGNVANKPTYFDIYTAGAGTGDVAVVIVDPQGRRDTVEVALEDKGDNTFRCTYRPVMEGPHTVHVAFAGAPITRSPFPVHVAEACNPNACRASGRGLQPKGVRVKEVADFKVFTKGAGSGELKVTVKGPKGTEEPVKVREAGDGVFECEYYPVVPGKYVVTITWGGYAIPRSPFEVQVSPEAGAQKVRAWGPGLETGQVGKSADFVVEAIGTEVGTLGFSIEGPSQAKIECDDKGDGSCDVRYWPTEPGEYAVHVICDDEDIRDSPFIAHIQPAPPDCFPDKVKAFGPGLEPTGCIVDRPAEFTIDARAAGKGDLKLYAQDADGCPIDIKVIPNGDGTFRCSYVPTKPIKHTIIVSWGGVNVPKSPFRVNVGEGSHPERVKVYGPGVEKTGLKANEPTYFTVDCSEAGQGDVSIGIKCAPGVVGPVEADIDFDIIKNDNDTFTVKYTPPGAGHYTIMVLFANQEIPASPFHIKVDPSHDASKVKAEGPGLSRTGVEVGKPTHFTVLTKGAGKAKLDVHFAGAAKGEAVRDFEIIDNHDYSYTVKYTAVQQGNMAVTVTYGGDPVPKSPFVVNVAPPLDLSKVKVQGLNSKVAVGQEQAFSVNTRGAGGQGQLDVRMTSPSRRPIPCKLEPGGGAEAQAVRYMPPEEGPYKVDITYDGHPVPGSPFAVEGVLPPDPSKVCAYGPGLKGGLVGTPAPFSIDTKGAGTGGLGLTVEGPCEAKIECQDNGDGSCAVSYLPTEPGEYTINILFAEAHIPGSPFKATIQPVFDPSKVRASGPGLERGKAGEAATFTVDCSEAGEAELTIEILSDAGVKAEVLIQNNADGTYHITYSPAFPGTYTITIKYGGHPIPKFPTRVHVQPAVDTSGIKVSGPGVEPHGVLREVTTEFTVDARSLTATGGNHVTARVLNPSGAKTDTYVTDNGDGTYRVQYTAYEEGVHLVEVLYDEVAVPKSPFRVGVTEGCDPTRVRAFGPGLEGGLVNKANRFTVETRGAGTGGLGLAIEGPSEAKMSCKDNKDGSCTVEYIPFTPGDYDVNITFGGQPIPGSPFRVPVKDVVDPGKVKCSGPGLGTGVRARVPQTFTVDCSQAGRAPLQVAVLGPTGVAEPVEVRDNGDGTHTVHYTPATDGPYTVAVKYADQEVPRSPFKIKVLPSHDASKVRASGPGLNASGIPASLPVEFTIDARDAGQGLLTVQILDPEGKPKKANIRDNGDGTYTVSYLPDMSGRYTITIKYGGDEIPYSPFRIHALPTGDASKCLVTVSIGGHGLGACLGPRIQIGEETVITVDAKAAGKGKVTCTVSTPDGAELDVDVVENHDGTFDIYYTAPEPGKYVITIRFGGEHIPNSPFHVLACDPLPHVEEPAEMLQMRQPYAPLRPGTCPTHWATEEPVVPVEPLESMLRPFNLVIPFTVQKGELTGEVRMPSGKTARPNITDNKDGTITVRYAPTEKGLHQMGIKYDGNHIPGSPLQFYVDAINSRHVSAYGPGLSHGMVNKPATFTIVTKDAGEGGLSLAVEGPSKAEITCKDNKDGTCTVSYLPTAPGDYSIIVRFDDKHIPGSPFTAKITGDDSMRTSQLNVGTSTDVSLKITEGDLSQLTASIRAPSGNEEPCLLKRLPNRHIGISFTPKEVGEHVVSVRKSGKHVTNSPFKILVGPSEIGDASKVRVWGKGLSEGQTFQVAEFIVDTRNAGYGGLGLSIEGPSKVDINCEDMEDGTCKVTYCPTEPGTYIINIKFADKHVPGSPFTVKVTGEGRMKESITRRRQAPSIATIGSTCDLNLKIPGNWFQMVSAQERLTRTFTRSSHTYTRTERTEISKTRGGETKREVRVEESTQVGGDPFPAVFGDFLGRERLGSFGSITRQQEGEASSQDMTAQVTSPSGKTEAAEIVEGEDSAYSVRFVPQEMGPHTVTVKYRGQHVPGSPFQFTVGPLGEGGAHKVRAGGTGLERGVAGVPAEFSIWTREAGAGGLSIAVEGPSKAEIAFEDRKDGSCGVSYVVQEPGDYEVSIKFNDEHIPDSPFVVPVASLSDDARRLTVTSLQETGLKVNQPASFAVQLNGARGVIDARVHTPSGAVEECYVSELDSDKHTIRFIPHENGVHSIDVKFNGAHIPGSPFKIRVGEQSQAGDPGLVSAYGPGLEGGTTGVSSEFIVNTQNAGSGALSVTIDGPSKVQLDCRECPEGHVVTYTPMAPGNYLIAIKYGGPQHIVGSPFKAKVTGPRLSGGHSLHETSTVLVETVTKSSSSRGASYSSIPKFSSDASKVVTRGPGLSQAFVGQKNSFTVDCSKAGTNMMMVGVHGPKTPCEEVYVKHMGNRVYNVTYTVKEKGDYILIVKWGDESVPGSPFKVNVP

SEQ ID NO: 37 - protein, 780 aa, Mus musculus
MAPYSLLVTRLQKALGVRQYHVASVLCQRAKVAMSHFEPSEYIRYDLLEKNINIVRKRLNRPLTLSEKIVYGHLDDPANQEIERGKTYLRLRPDRVAMQDATAQMAMLQFISSGLPKVAVPSTIHCDHLIEAQVGGEKDLRRAKDINQEVYNFLATAGAKYGVGFWRPGSGIIHQIILENYAYPGVLLIGTDSHTPNGGGLGGICIGVGGADAVDVMAGIPWELKCPKVIGVKLTGSLSGWTSPKDVILKVAGILTVKGGTGAIVEYHGPGVDSISCTGMATICNMGAEIGATTSVFPYNHRMKKYLSKTGRTDIANLAEEFKDHLVPDPGCQYDQVIEINLNELKPHINGPFTPDLAHPVADVGTVAEKEGWPLDIRVGLIGSCTNSSYEDMGRSAAVAKQALAHGLKCKSQFTITPGSEQIRATIERDGYAQILRDVGGIVLANACGPCIGQWDRKDIKKGEKNTIVTSYNRNFTGRNDANPETHAFVTSPEIVTALAIAGTLKFNPETDFLTGKDGKKFKLEAPDADELPRSDFDPGQDTYQHPPKDSSGQRVDVSPTSQRLQLLEPFDKWDGKDLEDLQILIKVKGKCTTDHISAAGPWLKFRGHLDNISNNLLIGAINIENGKANSVRNAVTQEFGPVPDTARYYKKHGIRWVVIGDENYGEGSSREHAALEPRHLGGRAIITKSFARIHETNLKKQGLLPLTFADPSDYNKIHPVDKLTIQGLKDFAPGKPLKCVIKHPNGTQETILLNHTFNETQIEWFRAGSALNRMKELQQ

SEQ ID NO: 38 - protein, 110 aa, Mus musculus
MSMTDVLSAEDIKKAIGAFAAADSFDHKKFFQMVGLKKKNPDEVKKVFHILDKDKSGFIEEDELGSILKGFSSDARDLSAKETKTLLAAGDKDGDGKIGVEEFSTLVAES

SEQ ID NO: 39 - protein, 142 aa, Mus musculus
MVLSGEDKSNIKAAWGKIGGHGAEYGAEALERMFASFPTTKTYFPHFDVSHGSAQVKGHGKKVADALASAAGHLDDLPGALSALSDLHAHKLRVDPVNFKLLSHCLLVTLASHHPADFTPAVHASLDKFLASVSTVLTSKYR

SEQ ID NO: 40 - protein, 142 aa, Mus musculus
MSLMKNERAIIMSMWEKMAAQAEPIGTETLERLFCSYPQTKTYFPHFDLHHGSQQLRAHGFKIMTAVGDAVKSIDNLSSALTKLSELHAYILRVDPVNFKLLSHCLLVTMAARFPADFTPEVHEAWDKFMSILSSILTEKYR

SEQ ID NO: 41 - protein, 646 aa, Mus musculus
MSKGPAVGIDLGTTYSCVGVFQHGKVEIIANDQGNRTTPSYVAFTDTERLIGDAAKNQVAMNPTNTVFDAKRLIGRRFDDAVVQSDMKHWPFMVVNDAGRPKVQVEYKGETKSFYPEEVSSMVLTKMKEIAEAYLGKTVTNAVVTVPAYFNDSQRQATKDAGTIAGLNVLRIINEPTAAAIAYGLDKKVGAERNVLIFDLGGGTFDVSILTIEDGIFEVKSTAGDTHLGGEDFDNRMVNHFIAEFKRKHKKDISENKRAVRRLRTACERAKRTLSSSTQASIEIDSLYEGIDFYTSITRARFEELNADLFRGTLDPVEKALRDAKLDKSQIHDIVLVGGSTRIPKIQKLLQDFFNGKELNKSINPDEAVAYGAAVQAAILSGDKSENVQDLLLLDVTPLSLGIETAGGVMTVLIKRNTTIPTKQTQTFTTYSDNQPGVLIQVYEGERAMTKDNNLLGKFELTGIPPAPRGVPQIEVTFDIDANGILNVSAVDKSTGKENKITITNDKGRLSKEDIERMVQEAEKYKAEDEKQRDKVSSKNSLESYAFNMKATVEDEKLQGKINDEDKQKILDKCNEIISWLDKNQTAEKEEFEHQQKELEKVCNPIITKLYQSAGGMPGGMPGGFPGGGAPPSGGASSGPTIEEVD

SEQ ID NO: 42 - protein, 182 aa, Mus musculus
MGDEEKRNRAITARRQHLKSVMLQIAATELEKEESRRESEKENYLSEHCPPLHIPGSMSEVQELCKQLHAKIDVAEEEKYDMEVKVQKSSKELEDMNQKLFDLRGKFKRPPLRRVRMSADAMLKALLGSKHKVCMDLRANLKQVKKEDTEKERDLRDVGDWRKNIEEKSGMEGRKKMFESES

SEQ ID NO: 43 - protein, 405 aa, Mus musculus
MRATDRMGARAVSELRLALLFVLVLGTPRLGVQGEDGLDFPEYDGVDRVINVNAKNYKNVFKKYEVLALLYHEPPEDDKASQRQFEMEELILELAAQVLEDKGVGFGLVDSEKDAAVAKKLGLTEEDSVYVFKGDEVIEYDGEFSADTLVEFLLDVLEDPVELIEGERELQAFENIEDEIKLIGYFKSKDSEHYKAYEDAAEEFHPYIPFFATFDSKVAKKLTLKLNEIDFYEAFMEEPMTIPDKPNSEEEIVSFVEEHRRSTLRKLKPESMYETWEDDLDGIHIVAFAEEADPDGYEFLETLKAVAQDNTENPDLSIIWIDPDDFPLLVPYWEKTFDIDLSAPQIGVVNVTDADSIWMEMDNEEDLPSADELEDWLEDVLEGEINTEDDDDDDDDDDDDDDDDD

SEQ ID NO: 44 - protein, 8886 aa, Mus musculus
MDHSFSGAPRFLTRPKAFVVSVGKDATLSCQIVGNPTPHVSWEKDRQPVEAGARFRLAQDGDVYRLTILDLALGDSGQYVCRARNAIGEAFAAVGLRVDSEGTCAEQAPHFLLRPTSIRVREGADATFRCRVGGSPQPAVSWSKDGRRLGPPDAPHVRVEEHGESSALRIRSARPRDGGTYEVRAENPLGSASAAAALVVDSDAEVAGPPGTSTATLLAHLQQRREAMRAEGIPPSPPGAGTRTCTVTEGKHARLSCFVTGEPKPETVWKKDGQLVTEGRRHVVYEDEQENFVLKILFCKQSDRGLYTCTASNLVGQTYSSVLVVVREPTVPFKKRLQDLEVREKESATFQCEVAQPATEAAWFKEETRLWASAKYDIEEEGTERRLTVRNVSADDDAVYICETTEGSRTVAELSVQGNLTRKLPRKTAVRTGDTAIFWVELAVPEGPVRWLRNQEEMVAGGRVAITAEGTCHTLTIFQCTLEDMGEVAFVSGGCRTTTQFCVSAPRRPPLYPPADPVVKAKTESSVTLSWSAPPHGDRPVTIDGYVLEKRKLGAYAWSRCHEAGWLATTEFTITGVAEEGDFQFRVSAINHFGQGPYLEFPGTMHLVPMLAVKTPLKAVEAVEGGEVTFSVDLTVASSGEWFLDGEALKASSIYVIRCDRTRHMLTIREVPARLHGAQLKFVANGIETSIQMVVRAALGLPSSKLPAAAAREVLAQLHEEAQLLAELSDQAAAVTWLKDGRELSLGPKYEMQVSAGRRALLVRDVAQDDAGLYECVSRGSRTAYQLLVQDITDGYRDWGPAGPQKHMCKCAGAKIARYLGSSCYRFLQYDKGVWHWLEAALDTRQGKGTSSCSLHEKPKLVFAKGQQAHSEVKAEAGNSATLSCEVTQAQTEVTWFKDGKKLSSSSKVRMEASGCSRRLVVQQAGKADAGEYSCEAGGQKLSFRLDVAEPKLVFAKGQQAHSEVKAEAGASATLSCEVAQAQTEVTWFKDGKKLSSSSKVRMEASGCSRRLVVQQAGKADAGEYSCEAGGQKLSFRLDVAEPKMVFAKEQQARSEVKAEAGASATLSCEVAQAQTEVTWFKDGKKLSSSSKVRMEASGCSRRLVVQQAGKADAGEYSCEAGGQKLSFRLDVTEPKLVFAKEQQARSEVKAEVGNSATLSCEVAQAQTEVTWFKDGKKLSSSSKVRMEASGCSRRLVVQQAGKADAGEYSCEAGGQKLSFRLDVAEPKLVFAKEQQARSEVKAEAGNSATLSCEVAQAQTEVTWFKDGKKLSSSSKVRMEASGCSRRLVVQQAGKADAGEYSCEAGGQKLSFHLDVTEPKLVFAKEQQAHSEVKAEAGASATLSCEVAQAQTEVTWFKDGKKLSSSSKVRMEASGCSRRLVVQQAGKADAGEYSCEAEGQKLSFRLDVAEPKLVFAKEQQARSEVKAEAGASATLSCEVAQAQTEVTWFKDGKKLSSSSKVRVEASGCSRRLVVQQAGKADAGEYSCEAGGQKLSFRLDVAEPKLVFAKEQQANSEVKAEAGASATLSCEVAQAQTEVTWFKDGKKLSSSSKVRVEASGCSRRLVVQQAGKADAGEYSCEAGGQKLSFRLDVAEPKLAFAKEQQAHSEVKAEAGASATLSCEVAQAQTEVTWFKDGKKLSSSSKVRVEASGCSRRLVVQQAGKADAGEYSCEAGGQKLSFRLDVAEPKLAFAKEQQAHSEVKAEAGASATLSCEVAQAQTEVTWFKDGKKLSSSSKVRVEASGCSRRLVVQQAGKADAGEYSCEAGGQKLSFRLDVAEPKLAFAKEQQAHSEVKAEAGASATLSCEVAQAQTEVTWFKDGKKLSSSSKVRVEASGCSRRLVVQQAGKADAGEYSCEAGGQKLFFRLDVAEPKLMFAKEQQAHSEVKAEAGASATLSCEVAQAQTEVTWFKDGKKLSSSSKVRVEASGCSRRLVVQQAGKADAGEYSCEAGGQKLSFRLDVAEPKLVFAKEQQAHSEVKAEAGASATLSCEVAQAQTEVTWFKDGKKLSSSSKVRVEASGCSRRLVVQQVGKADAGEYSCEARGQKLSFRLDVADTRLMFAKEQQARTEVKAEAGNSATLSCEVAQAQTEVTWFKDGKKLSSSSKVRVEASGCSRRLVVQQAGKADAGEYSCEAGGQKLSFRLDVAEAESQIPERPSRREPLVVKEHETIILTATIAAPSVAAVTWLKDGVEIRRSKRHEATSLGDTHTLTVRGAQVLDSAIYSCRVGKEGQDFPVQVEEVAAKFSKPLEPVEGELGGTVMLVCELSPEQAEVVWRCGNTQLRPGKRFQMTSEGPRRTLTVSGLREDDAEEYVCESRDDRTSARLTVKVPRVVKFTSGLSAMVAEEGQEATFQCVVSPSDAGVTWYKDGMQLQPSEKFVMVESGASRSLTILGLTLEDAGQVTVEAEGASSSAALRVREAPVLFKKKLEPQTVEERTSVTLEVELTRPWPEVKWTRNAAVLTPSENVEIRAEGARHCLVLRSVGFADRGFFGCETPDDKTQAKLNVEMRQVRLVRGLQEVEAKEQGTASMDVELSHAEVEGSWTRDGLRLQPGPKCHLAVQGPVHILTLSALQPQDSGLVAFRAEGVHTSARLIVTELPVSFTRVLQDVVATQKEKVTLECELSRPVDVRWLKDGVELRAGKAIGIVAQGTCRSLVIYRCETGDQGVYVCDALDAQTSASLRVQGRTYTLIFRRVLAEDAGEVKFVAENAESRAHLRVKELPVTLLRPLRDKIAMEKHRGVLECQVSRASAQVRWFKGGVELQSGPKYEVVSDGLYRKLVINDVQPEDEDTYTCDAGNVKTSAQFFVEEQSITIVRGLKDMTVMEPAPAWFECETSIPSVRPPKWLLGKTVLQAGGNVGLEQDGTVHRLTLHKTCSTMTGPVHFTIGKSRSSAQLVVSDIPVVLTRPLEPKAGRELQSVVLSCDFRPAPKAVQWYKDDTPLSPSEKFKMALEGQMAELRILRLTPADAGVYRCQAGSAQSSAEVTVEAREVTVIQPLQDAEAMEEGRVCFSCELSHKDEDIEWSLNGTPLYNDSFHEISHEGCLHTLVLKSVRQADTGTVCATSPKVSVSARLVVKGKPVVFLKALDDVSAEERGTLTLQCEVSDPEARVVWRKDGVELGPSDKYDFLHKAGARGLTVHDLSHEDAGLYTCQVGSKETQSKVSVHDLHVGITKRLKTVEVLEGESCSFECVLSHESPSDPAVWTVGGKTVGGSGHFHAVRQGRKYTLTVKDAALSDAGEVVFSVLGLTSKASLIIRERPVDITKPLEDQRTTLGEDVMLSCELSRAGTSVRWLKDGKAIRKSQKYDLLSEGTRAVLVVRKASLKDSGEYTCETEASKSTAKLCVEEKANRFTEELADLQVEEKGRAVFTCKTEHPASVVTWRKGLLELRASGKHVPSQEGLTLKLTINALERTDSDTYTCDIGQARTQARLLVHGQKVRVIEDLEDTAVQEGSSAKFCCRIAPADYGPVHWFLDKTPLHSNELNEITVQPGGYHVLTLRQLALKDSGTVYFEAGDQRTSAALRVTEKPSIFSRPLTDVTVTEGEDLTLVCETTTVDSSVRWTKDGKTLRPSARCQLSHEGCQAQLLITATTPQDGGRYKCEIGGASSSSIVRVHALPVRFRESLKDVEVPEGKAATLRCVLSSVAAPVEWRHGDDVLKSSNKYSLRQEGAVLELVIRDLQPQDSGQYSCSFGDQTTSATLTVKTSSAQFVGKLRNKEATEGTTVTLRCELTKEAPVEWKKGTETLRNGDKYSLKQDGAVCELQICSLLVADAGEYSCVCGQEKTSATLTVKALLVHFVRRLRSKEATEGDTTTLQCELSKAAPVEWRKGTETLRDGDRYSLKQDGAVCELQIRSLTIADAGEYLCTCGQEKTSATLTVRALPAKFKDSLKNEEATEGTTATLSCELSKAAPVTWKKGPKTLQSGDKYVLRQDGAVCGLQIHGLTMADAGEYSCVCGQEKTSATLTVRGLPAKFIEDLRSQEATEGATAILRCELSKAAPVEWRKGSETLKDGDRYTLRQDGAVCELQIRGLAVVDTGTYSSLPTKFTEGLRNEEATEGTMATLRCQMSKAAPVEWRKGSETLRDGDRYSLRQDGAMCELQIRGLTIEDSGEYTCVCGQEKTSATLSVKALPSRFIEDLRSQEATEGTMATLRCQMSKTAPVEWKKGSETLRDGGRYSLRQDGPVCELQICDLAVEDAGEYSCVCGQEKTSATLSIKALPPRFIEDLRSQEATEGTMATLRCQMSKAAPVEWRKGSETLGDGGRYSLRQNGAVCELQIHDLAVEDTGEYSCVCGQEKTSATLNVKALPPRFIEDLRSQEATEGTMATLRCQMSKAAPVEWRKGSETLRDGGRYSLRQDGAVCELQIHDLDVEDAGQYSCVCGQEKTSAVLTVDALPPKFTEGLKKEEATEGTMVTLRCQMSKEATVEWRKGAKTLSDGGRYSLRQDGAMCELQICGLAVEDAGEYSCVCGQEKTSATLSVKALPPRFIEDLRSQEATEGTMATLRCQMSKAAPVEWRKGSETLRDGDRYSLRQDGAVCELQIRDLAVEDAGEYLCVCGQEKTSATLSVKALPPRFIEDLRSQEATEGTMATLRCQMSKAAPVEWRKGSKTLRDGDRYSLRQDGAMCELQICDLAVEDTGDYSCVCGQEKTSATLSVKALPPRFIEDLRSQEAREGTVATLRCRMSKAAPVEWRKGSETLKDGDRYSLRQEGNLCELQIRDLAVEDTEEYSCVCGQEKTSATLSVKALPAKFIEDLRSQEAPESSTVTLRCKLSKKASVVWKKGSETLRNGARYSLRQDGAVCELEIRDLTVEDTGEYSCTCGQERTSATLSIMAPQVVFQQPLQNLQAEEGSMASLRCELSVPNAAMVWSKGGLELQGDTRREARQQGCVAELLLRDLRREDAGEYSCTCGSQTTSATLMVTAAPVRFLRELQAQDVDEGATARLRCELSREAVSVEWRKGSLQLFPCAKYQMVQEGTTAELLVHGVEQEDAGEYTCDAGHTQSIARLSVRAPKPKFKTDLQSTEQEAGGTARLCCQLSEAEPGTPVQWLKEGVELHVGSKYEMRRQGAVCELLIHGLEAKDTGEYACLVGGQKTLASLRVKEPEVTIVRGLVDMEVQADEDVEFTCKVSQAGATDVQWHLQGLPLQSNEVTEVAVLADGCTHVLQLKGVTLEDAGTVSFHVGGLSSSAQLTVRVPEVTVLEPLKDVQLSEGQDAHFRCRLSRASGQEARWALGGVPLQCNEMNDITVEQGTLYLLTLHKVTLEDAGTITLQVGSCSSEAQLKVTAKNTVLRGLENVDALEGGEALFECQLSQPEVAAHTWLLDDEPVHTSEKVEVVYFENGLRHLLLLKNLKPQDSCRVTFLAGDVVTSAFLTVRGWRLEVLEPPHDASVKAGMQVRFTCILSEAVPVGEATWYINGAAIQPDDTDWTVTTDGSHHALTLSNAQPQHAGEVTFAARDAVASARLSVLALPDPPEDAEVVGRSDHSVTLSWVAPMSDGGGGLCGYRVEMKEASTGQWQLCHDLVPGPECVVDDLVPGKTYRFRVAAVGPAGAGEPVHLPQMVKIAPAPAPAPAPAPAPAPETRQAVVGEDICLELEVAADGGEVVWHKGTERIQPGGHFEVLSRGQRQMLVIKGFRTEDQGEYRCGPIQGLPSSGAATFNVVMTSGSGDEVPAQPSLPPEAAQEGDLHLLWEALARKRRMSREPTLDSISELPEEDSRVQHLRQEAEETAPDLSEGYSTADELARTGEADLSHTSSDDESRAGTPSLVTYLKKAGGPGISPLASKHEAQVTTSVKPQKQQEPVVPTCPPPGDLSAADLMDPSLDKAAVKIQAAFKGYKVRKEMKQQEGPVFSRTFGDTEAQVGDVLRLECVLATKTDMRACWLKDGIELTDGRHYHIDQLKDGTCSLLVTGLAPTDSGRYTCQVSTKSGRVSHSACVVVSGTESEAESSSGGELDDAFRRAARRLHRLFRTKSPAELSEEELFLSADEGPGEPEEPADWQTYREDENFVCIRFESLAEARQAVTCFRNMFATMGIGVEISLGEQGPRGVEMRIGKVAPTVTPAVPLAKTPGLQTSDAAPVFLTELQNQDVQDGYPMSFDCVVTGQPVPSVRWFKDGKLLEEDDHYMINEDQQGGHQLIITAVVPADMGVYRCLAENSMGVSSTKAELRVELTSTDYDTAADATETSSYFSAQGYLSSREQEGTESDEGQLPQVLEELKDLQVAPGTRLAKFQLKVKGYPAPKLYWFKDGQPLTTSDHIRMTDKKTLHTLEIVSVTREDSGQYAAYISNAVGAAYSSARLLVRGPSEPEEKPASDVHERLVPPRILEKFTPKKVKRGSSITFSVKVEGHPAPSVHWLKEEAEKGVLWIGPDTPGYTMASSSKQHSLVLLDVGRQHQGTYTCIATNPAGQALCSASLHISGLAKEEEQERVKEALISSFLQGTSQAVSAQMSESAGFADLVGQSKGESLVAEEAHSHLSLAEVGTEEFLQKLTSQITEMVSAKISQAKLQVPGGDSDEETKTPSASPRHGRSRPSSSVQESSSESEDGDSRGEIFDIYVVTADYLPLGAEQDAIILREGQYVEVLDSAHPLRWLVRTKPTKSSPSRQGWVSPAYLDKRLKLSPEWGPTEAPEFPGEAVSEDEYRTRLSSVIQELLSSEQAFVGELQFLESHHMKHLERSPRVPAAVASQKTVIFRNVQDISHFHSSFLKELQGCGTDDDVAMCFIKNQEAFEKYLEFLVGRVQAESVVVSTPVQEFYKKYAEETLSAKDPTQPPPPPLQHYLEQPVERVQKYQALLKELIRNKARNRQNCALLEQAYAVVSALPQRAENKLHVSLMENYPGTLEALGEPIRQGHFIVWEGAPGARMPWKGHNRHVFLFRNYLVICKPRRDSRTDTFSYVFRNMMKLSSIDLNDQVEGDDRAFEVWHEREDSVRKYLLQARTVIIKNSWVKEICGIQQRLAQPVWRPPEFEEELADCTAELGETVKLACRVTGTPKPIVSWYKDGKPVEVDPHHILIEDPDGSCTLILDNLTGIDSGQYMCFAASAAGNASTLGKILVQVPPRFVNKVRATPFVEGEDAQITCTVEGAPYPQIRWYKDGTLLAPGNRYRMLNEPRSGVLVLVIQAASKEDLGHYECELVNRLGSTRGGGELYMQSPALRARDQHHREQIVAAVEDTSVEGSAHSAQDGADQQAASVLWRLLGSEALGPSPGDLPNTRQSEPPAFEEAASQIPGAASGTPEVSQPGTHKGLEQETTSSGSQGWTVPIRVEGTAWPGAGTGQLLLDVHSQVIMETTQRTYVCQAPDTGVTRAPSMQVTIEDVQVQVGDMAQFDAVIEGHPPPIVTWYKGSTQLTSSARLSQRQDGTTYSLVLTDVAPHDAGVYTCVANNAGGQVLCKAELLVHGGDKLDAENQVYRRKLHSFYDVQEEIGRGVFGFVKRVQHKGNKMFCAAKFIPLRSKTRAQAYQERDILATLGHPLVTGLLDQFETRKTLILILELCSSEELLDRLFKKGVVTEAEVKVYIQQLVEGLHYLHSHGILHLDIKPPNILMVHPAREDIKICDFGFAQKITPSEPQYSKYGSPEFVSPEIIEQNPVSEGSDIWAMGVISYLSLTCSSPFAGESDRATLLNVLEGRVSWSSPTAAHLSEDAKDFIKATLQRTPRARPSTSQCLAHPWFLKSMPAEEAHFINTKQLKFLLARSRWQRSLMSYKSILVMRSIPELLQGPPDSPSLGVARHLRGEASGASSSSSSSDNELAPFARAKSLPPSPVTHSPLLHPRGFLRPSASLPEETEASMPTADAAVPASPQSAGPPASPGCVPRHSVISSLFYQQAGEGAERGNKTSGAKRHPARRRHLLKGGYIARALPGLREPLMEYSLLEEEAAREEQASLMTKTPSFETALRLPSSSVREVPGRSHSLDNPPVTTGPSPEACKEQLLFPPSTGLTHETTAKDRGHKEGFLQESVPFPPMSGDSRPGKQEGSSQDSCRGKPASSCHSELGSGSQEGCGPPSSQSLGSLPPQSLKKELSTSCGPLFSEQPQAAPFPTQVSPLLGSEKEPQDGSLSEGPVPVPSSSPGSASQVDASLDTEGLSEAGDTCDFTPPPQRPQEQATTRKFSLESRGGYAGVAGYGTFAFGGDAGGMLGQGPLWARMAWAVSQSSEEQDEAATESPQPLESLGPIAEASGVPLRTSPSLTPWEEVEQVSLVQIRDLSGDAEAADTISLDISEVDPAYLNLSDLYDIKYLPFEFMIFRRVPKPIEQPESPGSETEAGQGLADFLEEAAWPWPGELGLRAGLEITEEPEEPGDLEALLGEAAVGRKRKWSPSRGLFQFPGRCLSGEEPVELGLRQRVKASMAHISRILKGRPEGPEREGPPRKKAGLASFRLSGLKGRDQELSDEAVVLGQSVTLACQVLAQPTAQATWSKDGVLLESSGHLLISSTLKNFQLLTILVVKEEDLGTYTCCVSNPLGTAVTTGVLRKAERPSSSPRPEVGELYKDAVLLVWKPVESCGPVTYIVQCCIEGGSWTTLASDISDCCYLTGKLSRGGMYIFRTACVSKAGMGPYSSPSEQVLLGGPNHLASEEESSRGRPAQLLPSTKTFAFQMQIRRGRFSVVRQCREKASGRALAAKIVPYQPEDKTAVLREYEALKRLHHPHLAQLHAAYLSPRHLVLILELCSGPELLPSLAERESYSESDVKDYLWQMLSATQYLHAQHILHLDLRSENMMVTEYNLLKVIDLGNAQSLDQEKVPAPENFKDYLETMAPELLEGQGAVPQTDIWAIGVTAFIMLSGEYPESSEGTRDLQKGLRKGLIRLSRCYAGLSGGAVAFLQSSLCAQPWGRPCASTCLQCGWLTEEGPTGSRPTPVTFPTVRLRAFVREREKRRALLYKKHNLAQVR

SEQ ID NO: 45 - protein, 1532 aa, Mus musculus
MEHSKQIRILLLNEMEKLEKTLFRLEQGFELQFRLGPTLQGKAVTVYTNYPLPGEAFNREKFRSLDWENPTEREDDSDKYCKLHLQQSGSFQYYFLQGNEKSGGGYIVVDPILRIGVDNHVLPLDCVTLQTFLAKCLGPFDEWESRLRVAKESGYNMIHFTPLQTLGLSRSCYSLADQLELNPDFSRPSKRYTWSDVGQLVEKLKREWNILCITDVVYNHTATNSKWILEHPESAYNLVNSPHLKPAWVLDRALWHFSCDVADGKYREKGVPALIENDQHMNCIRKIIWEDIFPRIQLWEFFQVDVHKAVEQFRRLLSQENRRVTKSEPKEHLKIIQDPEYRRRGCAVDMDTALATFIPHDNGPAAIEECCNWFRKRLEELNSEKHHLTSCHQEQAVNCLLGNVFYERLAGHGPKLGPVTRKYPLVTRYFTFPFGEMALSAEEALIHLPDKACFLMAHNGWVMGDDPLRNFAEPGSDVYLRRELICWGDSVKLRYGNKPEDCPYLWAHMKKYTEITATHFQGVRLDNCHSTPLHVAEYMLDAARKLQPNLYVVAELFTGSEELDNIFVTRLGISSLIREAMSAYNSHEEGRLVYRYGGEPVGSFVQPCLRPLMPAIAHALFMDITHDNECPIVHRSAYDALPSTTVVSMACCASGSTRGYDELVPHQISVVAEERFYTKWNPGASPADTGDVNVHSGIIAARCAINRLHQELGAKGFIQVYVDQVDEDIVAVTRHSPSIHQSVVAVSRTAFRNPKTSFYSKEVPQMCIPGKIEEVVLEARTIERNTKPYKKDENSINGMPNMTVELREHIQLHESKIVRQAGVATKGPNEYIQEIEFENLSPGSVIIFRVSLDPHAQVAVGILRNHLTQFSSHFKSGSLAVDNADPILKIPFASIASKLTLAELNQVLYRCESEEQEDGGGCYDIPNWSSLKYAGLQGLMSVLAEIRPKNDLGHPFCENLRSGDWMIDYVSGRLISRSGSIAEVGKWLQAMFFYLKQIPRYLIPCYFDAILIGAYTTLLDVAWKQMSSFVQNGSTFVKHLSLGSVQMCGVGKCPCLPLLSPSLLDVPCRLNEITKEKEQCCASLAAGLPHFSSGLFRCWGRDTFIALRGMLLVTGRYLEARNIILAFASTLRHGLIPNLLGEGTYARYNCRDAVWWWLQCIQDYCRTVPNGLDILKCPVSRMYPTDDSAPLPAGTLDQPLFEVIQEAMQRHMQGIQFRERNAGPQIDRNMKDEGFNITAGIDEETGFVYGGNRFNCGTWMDKMGESDRARNRGIPATPRDGSAVEIVGLCKSAVRWLLELSKKNIFPYHEVRVKRHGKVVAVSYDEWNRKIQNNFEKLFHVSEDPSDPNEKHPNLVHKRGIYKDSYGASSPWCDYQLRPNFTIAMVVAPELFTAEKAWKALEIAEKKLLGPLGMKTLDPDDMVYCGVYDNALDNDNYNLARGFNYHQGPEWLWPIGYFLRAKLYFSKMMGPETAAKTVFLVKNVLSRHYVHLERSPWKGLPELTNENGQYCPFSCETQAWSMAVVLETLYDL

SEQ ID NO: 46 - protein, 463 aa, Mus musculus
MGKEKTHINIVVIGHVDSGKSTTTGHLIYKCGGIDKRTIEKFEKEAAEMGKGSFKYAWVLDKLKAERERGITIDISLWKFETTKYYITIIDAPGHRDFIKNMITGTSQADCAVLIVAAGVGEFEAGISKNGQTREHALLAYTLGVKQLIVGVNKMDSTEPAYSEKRYDEIVKEVSAYIKKIGYNPATVPFVPISGWHGDNMLEPSPNMPWFKGWKVERKEGNASGVSLLEALDTILPPTRPTDKPLRLPLQDVYKIGGIGTVPVGRVETGILRPGMVVTFAPVNITTEVKSVEMHHEALSEALPGDNVGFNVKNVSVKDIRRGNVCGDSKADPPQEAAQFTSQVIILNHPGQISAGYSPVIDCHTAHIACKFAELKEKIDRRSGKKLEDNPKSLKSGDAAIVEMVPGKPMCVESFSQYPPLGRFAVRDMRQTVAVGVIKNVEKKSGGAGKVTKSAQKAQKAGK

SEQ ID NO: 47 - protein, 413 aa, Mus musculus
MAPPSVFAQVPQAPPVLVFKLTADFRDDPDPRKVNLGVGAYRTDESQPWVLPVVRKVEQKIANDNSLNHEYLPILGLAEFRSCASRLVLGDNSLAIRENRVGGVQSLGGTGALRIGADFLGRWYNGTDNKNTPIYVSSPTWENHNAVFSAAGFKDIRPYCYWDAEKRGLDLQGFLNDLENAPEFSIFVLHACAHNPTGTDPTPEQWKQIAAVMQRRFLFPFFDSAYQGFASGDLEKDAWAIRYFVSEGFELFCAQSFSKNFGLYNERVGNLTVVGKESDSVLRVLSQMEKIVRITWSNPPAQGARIVAATLSDPELFKEWKGNVKTMADRILTMRSELRARLEALKTPGTWSHITEQIGMFSFTGLNPKQVEYLVNEKHIYLLPSGRINMCGLTTKNLDYVATSIHEAVTKIQ

SEQ ID NO: 48 - protein, 194 aa, Mus musculus
MEEKLKKAKIIFVVGGPGSGKGTQCEKIVQKYGYTHLSTGDLLRAEVSSGSERGKKLSAIMEKGELVPLDTVLDMLRDAMLAKVDSSNGFLIDGYPREVKQGEEFEQKIGQPTLLLYVDAGAETMTQRLLKRGETSGRVDDNEETIKKRLETYYNATEPVISFYDKRGIVRKVNAEGTVDTVFSEVCTYLDSLK

SEQ ID NO: 49 - protein, 558 aa, Mus musculus
MAALTRNPQFQKLLEWHRANSANLKLRELFEADPERFNNFSLNLNTNHGHILVDYSKNLVNKEVMQMLVELAKSRGVEAARDNMFSGSKINYTENRAVLHVALRNRSNTPIKVDGKDVMPEVNRVLDKMKSFCQRVRSGDWKGYTGKSITDIINIGIGGSDLGPLMVTEALKPYSKGGPRVWFVSNIDGTHIAKTLASLSPETSLFIIASKTFTTQETITNAETAKEWFLEAAKDPSAVAKHFVALSTNTAKVKEFGIDPQNMFEFWDWVGGRYSLWSAIGLSIALHVGFDHFEQLLSGAHWMDQHFLKTPLEKNAPVLLALLGIWYINCYGCETHALLPYDQYMHRFAAYFQQGDMESNGKYITKSGARVDHQTGPIVWGEPGTNGQHAFYQLIHQGTKMIPCDFLIPVQTQHPIRKGLHHKILLANFLAQTEALMKGKLPEEARKELQAAGKSPEDLEKLLPHKVFEGNRPTNSIVFTKLTPFILGALIAMYEHKIFVQGIMWDINSFDQWGVELGKQLAKKIEPELEGSSAVTSHDSSTNGLISFIKQQRDTKLE

SEQ ID NO: 50 - protein, 469 aa, Mus musculus
MSQAYSSSQRVSSYRRTFGGAPGFSLGSPLSSPVFPRAGFGTKGSSSSMTSRVYQVSRTSGGAGGLGSLRSSRLGTTRAPSYGAGELLDFSLADAVNQEFLATRTNEKVELQELNDRFANYIEKVRFLEQQNAALAAEVNRLKGREPTRVAELYEEEMRELRRQVEVLTNQRARVDVERDNLIDDLQRLKAKLQEEIQLREEAENNLAAFRADVDAATLARIDLERRIESLNEEIAFLKKVHEEEIRELQAQLQEQQVQVEMDMSKPDLTAALRDIRAQYETIAAKNISEAEEWYKSKVSDLTQAANKNNDALRQAKQEMMEYRHQIQSYTCEIDALKGTNDSLMRQMRELEDRFASEANGYQDNIARLEEEIRHLKDEMARHLREYQDLLNVKMALDVEIATYRKLLEGEESRINLPIQTFSALNFRETSPEQRGSEVHTKKTVMIKTIETRDGEVVSEATQQQHEVL

SEQ ID NO: 51 - protein, 419 aa, Mus musculus
MASAFSKLLTGRNASLLFTTLGTSALTTGYLLNRQKVSADAREQHKLFPPSADYPDLRKHNNCMAECLTPTIYAKLRNKMTPSGYTLDQCIQTGVDNPGHPFIKTVGMVAGDEESYEVFADLFDPVIKLRHNGYDPRVMKHPTDLDASKITHGQFDERYVLSSRVRTGRSIRGLSLPPACSRAERREVENVAITALEGLKGDLAGRYYKLSEMTEQDQQRLIDDHFLFDKPVSPLLTCAGMARDWPDARGIWHNYDKTFLIWINEEDHTRVISMEKGGNMKRVFERFCRGLKEVERLIQERGWEFMWNERLGYILTCPSNLGTGLRAGVHVRIPKLSKDPRFSKILENLRLQKRGTGGVDTAAVADVYDISNIDRIGRSEVELVQIVIDGVNYLVDCEKKLERGQDIKVPPPLPQFGRK

SEQ ID NO: 52 - protein, 3284 aa, Mus musculus
MRKHRHLPLVAVFSLLLSGIATTHAQQHGDVKNGAAADIVFLVDSSWSAGKDRFLLVQEFLSDVVESLAVGDNDFHFALVRLNGNPHTEFLLNTYHSKQEVLSHIVNMSYIGGSNQTGKGLEYVIHSHLTEASGSRAADGVPQVIIVLTDGQSEDGFALPSAELKSADVNVFAVGVEGADERALGEVASEPLSMHVFNLENVTSLHGLVGNLVSCIHSSVNPERAGDKESLKDITAQDSADIIFLIDGSQNTGNANFDVIRDFLVNVLERLSVGNQQVQVGVVQYSEEPITMFSLNSYPSKAAVLDAVKGLSLVGGESANIGQALDFVVENHFTRAGGSRVEEGVPQVLVLISAGPSSDEIRDSVVALKQASVFSFGLGAQAASRAELQHIATDDSLVFTVPEFRSFGDLQEQILPYLVGVAQRHIVLQPPAIVTQVMEVNKRDIVFLVDGSSSLGPSNFNAIRDFVTRVIQRLEIGQDLVQVSVAQYADTVKPEFYLNSYTNKRDAITAVRKMRALNGSALYTGSSLDFVRNNLFTSSAGHRAAEGVPKLLVLITGGKSLDEVSQPAQELKRGSIMALAVGSKAADEDELKEIAFDSSLVFIPAEFRPAPLQNMLPSLMAPLRTLTGTTEVHVNKRDIIFLLDGSDNVGKNNFPYVRDFVTNLVNSLDVGSDNIRVGLVQFSDTPVTEFSLDTYQTKSELLAHLRRLQLKGGSGLNAGSALSYIHANHFTEAGGSRTREHVPQLLLLLMAGPSEDAYLQAANALVRSGVLTFCVGTNRADKAELEHIAFNPSLVYLMDDFRSLPSLPQQLIQPLTTYVSGGVQEVPLSQPESKRDILFLFDGSVNVLGQFPAVRDFLYRIIEELDVKPDGTRVAIAQFSDDVRLESRFSEHQTKAEILNLVKKMKLKTGKALNLGYALDYALRNIFVRSAGSRIEDNVQQFLVLLVAGRSSDAVAGPASSLKQRGVVPFIFQAKNANPSELEQIVLSPAFILAAESLPKIGDLQSQIVSLLKAEQGSGPVSGEKDVVFLIDGSEGVRSGFPLLKDFVQRVVESLDVGPDRVRVALVQYSDRTRPEFYLNSHMDQQGVISAIRRLTLLGGPTPNTGAALEFVLRNILTSSTGSRIAEGVPQLLIVLTAEPSGDDVRGPSVVLKQGGAVPIGIGIGNADISEMQTISFIPDFAVAIPTFRELGTIQQVISERVIQLNREELSSLKPILTPSTGAGVGSKKDVVFLIDGSRNAGPEFQYIRTLIERIVEYLDIGFDTTRVAVIQFSEDSKMEFPLNAHFSKDEVQNAVRRLRPKGGSQVYIGNALEYVLKNIFQRPLGSRIEEGVPQFLVLISSGKSDDEVDDSAVELKQFGVAPLTIARHTDQEELVKISLSPEYVYSVSTFRELPRLEQKLLTPITTLTSQQIHQILASTRYPPSVVESDAADIVFLIDSSDAVKPDGIAHIRDFVSRIVRRLNIGPSKVRIGVVQFSNDVFPEFYLKTHKSQSSVLEAIRRLRFKGGSPLNTGRALEFVARNLFVKSAGSRIEDGVPQHLVLFLGGKSQDDVARHAQVISSSGIVSLGIGDRNIDRTDLQTITNDPRLVFTVREFRELPNIEERVMLSFGPSGATPQPPGVDLPSPSRPEKKKADIVFLLDGSINFRRDSFQEVLRFASEIVDTVYEDGDSIRVGLVQYNSDPTDEFFLRDFSTKRQIIDAINKVVYKGGRHANTRVGIEHLLRNHFVPEAGSRLDERVPQIAFVITGGKSVEDAQDVSLALTQKGVKVFAVGVRNIDSEEVGKIASNSATAFRVGSVQELSELSETVLETLHDAMHETLCPGVTDVSKACNLEVILGFDGSRDQNVFVSQKGLESKVDIILNRISQIQRISCSGNQLPTVRVSVMANTPSGPVEAFDFAEYQPELFEKFRNMRSQRPYVLTADTLKLYQNKFRQSSPDTVKVVIHFTDGADGDMADLYRASEELRQAGAQALILVGLERVANLERLMHLEFGRGFMYDRPLRLNLLDLDYELAEQLDNIAEKACCGVPCKCSGERGDRGPIGSIGPKGISGEDGYRGYPGDEGGPGERGPPGVNGTQGFQGCPGQRGVKGSRGFPGEKGELGEIGLDGLDGEEGDKGLPGSSGEKGSPGRRGDKGPKGDKGERGDVGIRGDPGDSGRDSQQRGPKGETGDIGPMGLPGRDGIPGSPGDPGKDGGSGRRGPAGAKGNRGGPGQPGFEGEQGTRGSQGPPGPIGPPGLIGEQGIPGPRGGGGTAGAPGERGRTGPLGRKGEPGEPGPKGSIGNRGPRGETGDDGRDGVGSEGRRGKKGERGFPGYPGPKGTPGEPGADGPPGPKGIRGRRGNSGPPGATGQKGDPGYPGPSGHKGNRGDSVDQCALIQSIRDKCPCCYGPLECPVFPTELAFALDTSEGVTQDTFSRMREVLLGIVGDLTIAESNCPRGARVAVVTYNNEVTTEIRFADSKKKSALLDSIQNLQVALTSKQQSLETAMSFVARNTFKRVRSGFLMRKVAVFFSNKPTRASPQLREAVLKLSDAGITPLFLTSQEDRQLINALQINNTAVGHALVLPARRDLTDFLKNVLTCHVCLDICNIDPSCGFGSWRPSFRDRRAAGSDVDIDLAFILDSSEATTLFQFNEMKKYIGYVIRQLDLSPDPKASQHFARVAVVQQSTYESVDNASVPPVKVEFSLTDYGAKEKLLDFLSRRMTQLQGTMGLGNAIEYTIENIFESAPNPRDLKIMVLMLTGDMQRQQLEEAQRAILQAKCKGYFFVVLGIGRKVNIKEVYSFASEPNDVFFKFVDKSTELNEEPLMRFGRLLPSFVSSENAFYLPPDLRKQCDWFQGDQPVKNGVKFGHKQINPPHTANSSLTSKVVTTMKPVTTTKPTAIVNLPPAKPAPARPAPAQPVLAKPDPAKPAQARPAPAKPASAKLVPPQPVHVQPAPAQTASVRPAPAKPAPPQPAAAKPVPAKPAVPAQPAPPQPAAAKPVPAKPAVPAQPAPPQPAAAKPVPAKPAVPAQPAAAQPMPAQPVLTKSAAVKPASANKPVAAKPVATNTATATARPALAAKPAAAKPAATRPLAAAIRPVATKPEAPRQQAKPAATKPATTKPLARVSREVQVSEVTENSARLHWERPEPSSSFFYDLTVTSAHDQSLVLRQNLTVTDRVIGGLLAGQLYHVVVVSYLQSQVRAIYQGSFNTKKTQPPPLQAAHRASSSTINLMVNTEPLFLTKTDICKLSRDAGTCVDFKLLWHYDLESKSCKRFWYGGCGGNENRFHSQEECEKMCSPDLLV

SEQ ID NO: 53 - protein, 745 aa, Mus musculus
MPLFKLTGQGKQIDDAMRSFAEKVFASEVKDEGGRHEISPFDVDEICPISLHEMQAHIFHMENLSMDGRRKRRFQGRKTVNLSIPQSETSSTKLSHIEEFISSSPTYESVPDFQRVQITGDYASGVTVEDFEVVCKGLYRALCIREKYMQKSFQRFPKTPSKYLRNIDGEALVGNESFYPVFTPPPKKGEDPFRTEDLPANLGYHLKMKAGVIYIYPDEAAANRDEPKPYPYPNLDDFLDDMNFLLALIAQGPVKTYAHRRLKFLSSKFQVHQMLNEMDELKELKNNPHRDFYNCRKVDTHIHAAACMNQKHLLRFIKKSYHIDADRVVYSTKEKSLTLKELFAKLNMHPYDLTVDSLDVHAGRQTFQRFDKFNDKYNPVGASELRDLYLKTDNYINGEYFATIIKEVGADLVEAKYQHAEPRLSIYGRSPDEWNKLSSWFVCNRIYCPNMTWMIQVPRIYDVFRSKNFLPHFGKMLENIFLPVFEATINPQAHPDLSVFLKHITGFDSVDDESKHSGHMFSSKSPKPEEWTMENNPSYTYYAYYMYANITVLNSLRKERGMNTFLFRPHCGEAGALTHLMTAFMIADNISHGLNLKKSPVLQYLFFLAQIPIAMSPLSNNSLFLEYAKNPFLDFLQKGLMISLSTDDPMQFHFTKEPLMEEYAIAAQVFKLSTCDMCEVARNSVLQCGISHEEKAKFLGNNYLEEGPVGNDIRRTNVAQIRMAYRYETWCYELNLIAEGLKATE

SEQ ID NO: 54 - protein, 379 aa, Oryctolagus cuniculus
FGNTHNKYKLNYKSEEEYPDLSKHNNHMAKVLTPDLYKKLRDKETPSGFTLDDVIQTGVDNPGHPFIMTVGCVAGDEESYTVFKDLFDPIIQDRHGGFKPTDKHKTDLNHENLKGGDDLDPHYVLSSRVRTGRSIKGYTLPPHCSRGERRAVEKLSVEALNSLTGEFKGKYYPLKSMTEQEQQQLIDDHFLFDKPVSPLLLASGMARDWPDARGIWHNDNKSFLVWVNEEDHLRVISMEKGGNMKEVFRRFCVGLQKIEEIFKKAGHPFMWNEHLGYVLTCPSNLGTGLRGGVHVKLAHLSKHPKFEEILTRLRLQKRGTGGVDTAAVGSVFDISNADRLGSSEVEQVQLVVDGVKLMVEMEKKLEKGQSIDDMIPAQK

SEQ ID NO: 55 - protein, 900 aa, Oryctolagus cuniculus
MMMVMQPEGLGAREGPFAGGGGGGEYMEQEEDWDRDLLLDPAWEKQQRKTFTAWCNSHLRKAGTQIENIEEDFRNGLKLMLLLEVISGERLPRPDKGKMRFHKIANVNKALDFIASKGVKLVSIGAEEIVDGNLKMTLGMIWTIILRFAIQDISVEETSAKEGLLLWCQRKTAPYRNVNVQNFHTSWKDGLALCALIHRHRPDLIDYAKLRKDDPIGNLNTAFEVAEKYLDIPKMLDAEDIVNTPKPDEKAIMTYVSCFYHAFAGAEQAETAANRICKVLAVNQENEKLMEEYEKLASELLEWIRRTVPWLENRVGEPSMSAMQRKLEDFRDYRRLHKPPRVQEKCQLEINFNTLQTKLRLSHRPAFMPSEGKLVSDIANAWRGLEQVEKGYEDWLLSEIRRLQRLQHLAEKFQQKASLHETWTRGKEDMLTQRDYESASLQEVRALLRRHEAFESDLAAHQDRVEHIAALAQELNELDYHEAASVNSRCQAICDQWDNLGTLTQKRRDALERMEKLLETIDQLQLEFARRAAPFNNWLDGAIEDLQDVWLVHSVEETQSLLTAHDQFKATLPEADRERGAILGIQGEIQKICQTYGLRPSSTNPYINLSPQDINTKWDTVRKLVPSRDQTLQEELARQQMNERLRRQFAAQANAVGPWIQAKVEEVGRLAAGLAGSLEEQMAGLRQQEQNIINYKSNIDRLEGDHQLLQESLVFDNKHTVYSMEHIRVGWEQLLTSIARTINEVENQVLTRDAKGLSQEQLNEFRASFNHFDRKRNGMMEPDDFRACLISMYDLGEVEFARIMTMVDPNAAGVVTFQAFIDFMTRETAETDTAEQVVASFKILAGDKNYITPEELRRELPAEQAEYCIHRMAPYKGAGAPAGALDYVAFSSALYGESDL

SEQ ID NO: 56 - protein, 842 aa, Oryctolagus cuniculus
SRPLSDQEKRKQISVRGLAGVENVTELKKNFNRHLHFTLVKDRNVATPRDYYFALAHTVRDHLVGRWIRTQQHYYEKDPKRIYYLSLEFYMGRTLQNTMVNLALENACDEATYQLGLDMEELEEIEEDAGLGNGGLGRLAACFLDSMATLGLAAYGYGIRYEFGIFNQKICGGWQMEEADDWLRYGNPWEKARPEFTLPVHFYGRVEHTSQGAKWVDTQVVLAMPYDTPVPGYRNNVVNTMRLWSAKAPNDFNLKDFNVGGYIQAVLDRNLAENISRVLYPNDNFFEGKELRLKQEYFVVAATLQDIIRRFKSSKFGCRDPVRTNFDAFPDKVAIQLNDTHPSLAIPELMRVLVDLERLDWDKAWEVTVKTCAYTNHTVLPEALERWPVHLLETLLPRHLQIIYEINQRFLNRVAAAFPGDVDRLRRMSLVEEGAVKRINMAHLCIAGSHAVNGVARIHSEILKKTIFKDFYELEPHKFQNKTNGITPRRWLVLCNPGLAEIIAERIGEEYISDLDQLRKLLSYVDDEAFIRDVAKVKQENKLKFAAYLEREYKVHINPNSLFDVQVKRIHEYKRQLLNCLHVITLYNRIKKEPNKFVVPRTVMIGGKAAPGYHMAKMIIKLITAIGDVVNHDPVVGDRLRVIFLENYRVSLAEKVIPAADLSEQISTAGTEASGTGNMKFMLNGALTIGTMDGANVEMAEEAGEENFFIFGMRVEDVDRLDQRGYNAQEYYDRIPELRQIIEQLSSGFFSPKQPDLFKDIVNMLMHHDRFKVFADYEEYVKCQERVSALYKNPREWTRMVIRNIATSGKFSSDRTIAQYAREIWGVEPSRQRLPAPDEKIP

SEQ ID NO: 57 - protein, 362 aa, Oryctolagus cuniculus
HSHPALTPEQKKELSDIAHRIVAPGKGILAADESTGSIAKRLQSIGTENTEENRRFYRQLLLTADDRVNPCIGGVILFHETLYQKADDGRPFPQVIKSKGGVVGIKVDKGVVPLAGTNGETTTQGLDGLSERCAQYKKDGADFAKWRCVLKIGEHTPSALAIMENANVLARYASICQQNGIVPIVEPEILPDGDHDLKRCQYVTEKVLAAVYKALSDHHIYLEGTLLKPNMVTPGHACTQKYSHEEIAMATVTALRRTVPPAVTGVTFLSGGQSEEEASINLNAINKCPLLKPWALTFSYGRALQASALKAWGGKKENLKAAQEEYVKRALANSLACQGKYTPSGQAGAAASESLFISNHAY

SEQ ID NO: 58 - protein, 191 aa, Oryctolagus cuniculus
APKKDVKKPAAAAAPAPAPAPAPAPAPAKPKEEKIDLSAIKIEFSKEQQDEFKEAFLLYDRTGDSKITLSQVGDVLRALGTNPTNAEVKKVLGNPSNEEMNAKKIEFEQFLPMLQAISNNKDQGTYEDFVEGLRVFDKEGNGTVMGAELRHVLATLGEKMKEEEVEALMAGQEDSNGCINYEAFVKHIMSI

SEQ ID NO: 59 - protein, 333 aa, Oryctolagus cuniculus
MVKVGVNGFGRIGRLVTRAAFNSGKVDVVAINDPFIDLHYMVYMFQYDSTHGKFHGTVKAENGKLVINGKAITIFQERDPANIKWGDAGAEYVVESTGVFTTMEKAGAHLKGGAKRVIISAPSADAPMFVMGVNHEKYDNSLKIVSNASCTTNCLAPLAKVIHDHFGIVEGLMTTVHAITATQKTVDGPSGKLWRDGRGAAQNIIPASTGAAKAVGKVIPELNGKLTGMAFRVPTPNVSVVDLTCRLEKAAKYDDIKKVVKQASEGPLKGILGYTEDQVVSCDFNSATHSSTFDAGAGIALNDHFVKLISWYDNEFGYSNRVVDLMVHMASKE

SEQ ID NO: 60 - protein, 284 aa, Oryctolagus cuniculus; identical to SEQ ID NO: 12

SEQ ID NO: 61 - protein, 1134 aa, Oryctolagus cuniculus
EAKPAARKAPKGKDAPKEEPPKEAAAEPPKEAPPEDQSPTAEEPTGIFLKKPDSVSVETGKDAVIMAKLNGKELPAKPTIKWFKGKWLELGSKSGARFSFKETHDAASNVYSLELHIGKVVLGDRGDYRLEVKAKDVCDSCSFNIDVEAPRQDSAGQSLESFKRAGDAKPDGAGELDFSGLLKKREVVEEEKKKKKDDDDLGIPPEIWELLKGAKKSEYERIAFQYGITDLRGMLKRLKKAKVEVKKSAAFTKKLDPAYQVDRGNKVKLVVEISDPDLPLKWFKNGQEIKPSLKYVFENVGKKRILTINKCTLADDAAYEVVVNDEKCFTELFVKEPPVLIVTPLEDQQVFVGDRVEMSVEVSEEGAQVMWMKDGVELTREGSFKSNYRFKKDGKRHILIYSEVNLEDRGHYQVMTNGGQCEAELIVEEKQLEVLQDIADLTVKASEQAMFKCEVSDEKVTGKWYKNGVEVRPSKRITISHVGRFHKLVIDDVRPEDEGDYTFVPDGYALSLSAKLNFLEIKVEYVPKQEPPKIHLDCSGKTSDNAIVVVAGNKLRLDVSITGEPPPVATWMKGDEVFTVTEGRVRIEQRPDSSSFVIESAERGDEGRYTIKVTNPAGEDVATVMLRVVDVPDPPEAVRVTSVGEDWAILVWDPPKYDGGQPVTGYLLERKKKGSQRWMKLNFEVFTETTYESTKMIEGILYEMRVFAVNAIGVSQPSMNTKPFMPIAPTSAPQHLTVEDVTDTTTTLKWRPPDRIGAGGIDGYLVEYCVEGSDEWIPANKEPVERCGFTVKGLPTGAKILFRVIGVNIAGRSEPATLVQPVTIREIVEQPKIRLPRHLRQTYVRKVGEHINLVIPFQGKPRPQVVWTKGGAPVDTSRVHVRTSDFDTVFFVREAARSDSGEYELSVQIENMKDTATVRIRVVEKAGPAENVMVKEVWGTNALVEWQPPKDNGNSEITGYLVQKADKKTMEWFTVYEHNRHTSCTVSDLIVGNEYYFRVYTENVCGLSDSPGVSKNTAHILKTGITFKPFEYKEHDFRSPPKFLTPLIDRVVVAGYAAALNCAVRGHPKPKVVWMKNKVEIHEDPKFLMTNHQGVLTLNIRRPSPFDAGTYSCRAFNELGEALAECRLDVRVPQ

SEQ ID NO: 62 - protein, 1415 aa, Oryctolagus cuniculus
MSLVAVPFYQKRHKHFDQSYRNIQTKYLMDEYTAKKRASTQMSTHKSASTQMATHKSALQKASSQTSSGGTTCRLCAKRLSAAEEEEQESKNRYRSLVASYGEAKRQRFLSELAQLEESVHLARSQARDVVDKYALQQMVEDKMAWERHVFEERLSRAPEILVQLRSHTVWEKMSVKLCFTVQGFPTPVVQWYKDGSLICQAGEPGKYRIDSKYGVHTLEISRASFDDTATYSAVATNAHGQVSTNAAVVVRRYRGDEEPFYSVGLPIGYVLVKESKWTRMFFGEGQASLSFSHLNKDDEGLYTLRIVSRGGVSDHSAFLFVRDADPLVTGAPGAPMDLQCHDANRDYVIVTWKPPNTTTESPVIGYFVDRCEVGTNNWVQCNDAPVKICKYPVTGLYEGRSYIFRVRAVNSAGISRPSRVSDAVAALDPADLRRLQAIHLEGEKEVVIYQDDLEGDVQVPGPPTNVHASEISRNYVVLSWEPPVPRGKEPLMYFIEKSVVGSGSWQRVNAQTAVRSPRYAVFDLAEGKPYVFRVLSANKHGLSDPSEITSPIQAQDTIAVPSAPGRVLASRNTKTSVVVQWDRPKHEEDLLGYYVDCCVAGTNAWEPCNHKPIGYNRFVVHGLTTGEQYIFRVKAVNALGTSENSQESEVIKVQAALTVPSHPYGVTLLNCDGHSMTLGWKVPKFSGGSPILGYYVDKREVHHKNWHEVNSSPIQERILTVEGLTEGSFYEFKIAATNLAGIGQPSDPSEHFKCEAWTAPEPGPAYDLTFCEVRDTSLVMLWKAPVYSGSSPVSGYFVDFKEEDSGEWSTVNEAATPNRYLKVSDLHQGRTYVFRVRAVNASGVGKPSDVSEPVLVEARPGTKEISAGVDEEGNIYLGFDCQEMTDASHFTWCKSYEEIADPERFHVETKGDHSKLYFKNPDKADLGTYSVSVSDTDGVSSSFVLDEEELERLLALSNEIKNPTIPLKSELAYEIFDKGQVRFWLQTEPLSPDASFRFVINDREVSDSETHRMKCDKASGIIEMVMDRFTIENEGTYTVQIHDGKAKNQSSLVLIGDAFRKVLEEAEFQRKEFLRKQGPHFAEYLHWDVTEECEVRLICKVANTKKETVFKWLKDDVLYETETPPDLEKGVCELLIPKLSKKDHGEYKATLKDERGQDASILEIAGKVYEDMILAMSRVCGMSASPLKILCTPEGIRLQCFMKYFTEEMRVNWYHKDAKISSSEHMRIGGSEDMAWLQICEPTEKDKGKYTFEIFDGKDNHQRSLDLSGQAFDEAFAEFQRLKAAAFAEKNRGKVIGGLPDVVTIMEGKTLNLTCTVFGNPDPEVVWFKNDQDIELSEHFSVKVEQAKYVSLTIKGVTSEDSGKYSINVKNKYGGEKIDVTVSVYKHGEAIPEVAPPQQAKPKLIPASASATAP

SEQ ID NO: 63 - protein, 433 aa, Oryctolagus cuniculus
AMQKIFAREILDSRGNPTVEVDLHTAKGRFRAAVPSGASTGIYEALELRDGDKSRYLGKGVLKAVEHINKTLGPALLEKKLSVVDQEKVDKFMIELDGTENKSKFGANAILGVSLAVCKAGAAEKGVPLYRHIADLAGNHDLVLPVPAFNVINGGSHAGNKLAMQEFMILPVGASSFREAMRIGAEVYHHLKGVIKAKYGKDATNVGDEGGFAPNILENNEALELLKTAIQAAGYPDKVVIGMDVAASEFHRNGKYDLDFKSPDDPARHITGQKLGELYKSFIKNYPVVSIEDPFDQDDWATWTSFLSGVDIQIVGDDLTVTNPKRIAQAVEKKACNCLLLKVNQIGSVTESIQACKLAQSNGWGVMVSHRSGETEDTFIADLVVGLCTGQIKTGAPCRSERLAKYNQLMRIEEALGDKAVFAGRKFRNPKAK

SEQ ID NO: 64 - protein, 169 aa, Oryctolagus cuniculus
APKKAKRRAAAEGGSSNVFSMFDQTQIQEFKEAFTVIDQNRDGIIDKEDLRDTFAAMGRLNVKNEELDAMMKEASGPINFTVFLTMFGEKLKGADPEDVITGAFKVLDPEGKGTIKKQFLEELLTTQCDRFSQEEIKNMWAAFPPDVGGNVDYKNICYVITHGDAKDQE

SEQ ID NO: 65 - protein, 1273 aa, Oryctolagus cuniculus
IFSISDNAYQFMLTDRENQSILITGESGAGKTVNTKRVIQYFATIAITGDKKKEEPTPGKMQGTLEDQIISANPLLEAFGNAKTVRNDNSSRFGKFIRIHFGATGKLASADIETYLLEKSRVTFQLKAERSYHIFYQIMSNKKPELIEMLLITTNPYDFAFVSQGEITVPSIDDQEELMATDSAVDILGFTSDEKVAIYKLTGAVMHYGNLKFKQKQREEQAEPDGTEVADKAAYLTSLNSADLLKALCYPRVKVGNEYVTKGQTVQQVYNAVGALAKAIYEKMFLWMVTRINQQLDTKQPRQYFIGVLDIAGFEIFDFNSLEQLCINFTNEKLQQFFNHHMFVLEQEEYKKEGIEWEFIDFGMDLAACIELIEKPMGIFSILEEECMFPKATDTSFKNKLYEQHLGKSNNFQKPKPAKGKAEAHFSLVHYAGTVDYNITGWLDKNKDPLNETVVGLYQKSGMKTLAFLFSGAQTGEEGGGGGKKGGKKKGSSFQTVSALFRENLNKLMTNLRSTHPHFVRCLIPNETKTPGAMEHELVLHQLRCNGVLEGIRICRKGFPSRILYADFKQRYKVLNASAIPEGQFIDSKKASEKLLGSIDVDHTQYKFGHTKVFFKAGLLGLLEEMRDEKLAQLITRTQAICRGYLTRVEFKKMMERRESIFCIQYNIRAFMNVKHWPWMKLYFKIKPLLKSAETEKEMANMKEEFEKTKEELAKSEAKRKELEEKMVALMQEKNDLQLQVQAEADGLADAEERCDQLIKTKIQLEAKIKEVTERAEDEEEINAELTAKKRKLEDECSELKKDIDDLELTLAKVEKEKHATENKVKNLTEEMAGLDENIAKLTKEKKALQEAHQQTLDDLQAEEDKVNTLTKAKTKLEQQVDDLEGSLEQEKKLRMDLERAKRKLEGDLKLAQESTMDIENDKQQLDEKLKKKEFEMTNLQSKIEDEQALAMQLQKKIKELQARIEELEEEIEAERASRAKAEKQRSDLSRELEEISERLEEAGGATSAQIEMNKKREAEFQKMRRDLEEATLQHEATAAALRKKHADSVAELGEQIDNLQRVKQKLEKEKSELKMEIDDLAGNMETVSKAKGNLEKMCRTLEDQLSEVKTKEEEHQRLINELSAQKARLHTESGEFSRQLDEKDAMVSQLSRGKQAFTQQIEELKRQLEEETKAKSALAHALQSSRHDCDLLREQYEEEQEAKAELQRAMSKANSEVSQWRTKYETDAIQRTEELEEAKKKLAQRLQDAEEHVEAVNSKCASLEKTKQRLQN

SEQ ID NO: 66 - protein, 1161 aa, Oryctolagus cuniculus
IFYQIMSNKKPELIEMLLITTNPYDFAFVSQGEITVPSIDDQEELMATDSAVDILGFTSDEKVAIYKLTGAVMHYGNLKFKQKQREEQAEPDGTEVADKAAYLTSLNSADLLKALCYPRVKVGNEYVTKGQTVQQVYNAVGALAKAIYEKMFLWMVTRINQQLDTKQPRQYFIGVLDIAGFEIFDFNSLEQLCINFTNEKLQQFFNHHMFVLEQEEYKKEGIEWEFIDFGMDLAACIELIEKPMGIFSILEEECMFPKATDTSFKNKLYEQHLGKSNNFQKPKPAKGKAEAHFSLVHYAGTVDYNITGWLDKNKDPLNETVVGLYQKSGMKTLAFLFSGAQTGEEGGGGGKKGGKKKGSSFQTVSALFRENLNKLMTNLRSTHPHFVRCLIPNETKTPGAMEHELVLHQLRCNGVLEGIRICRKGFPSRILYADFKQRYKVLNASAIPEGQFIDSKKASEKLLGSIDVDHTQYKFGHTKVFFKAGLLGLLEEMRDEKLAQLITRTQAICRGYLTRVEFKKMMERRESIFCIQYNIRAFMNVKHWPWMKLYFKIKPLLKSAETEKEMANMKEEFEKTKEELAKSEAKRKELEEKMVALMQEKNDLQLQVQAEADGLADAEERCDQLIKTKIQLEAKIKEVTERAEDEEEINAELTAKKRKLEDECSELKKDIDDLELTLAKVEKEKHATENKVKNLTEEMAGLDENIAKLTKEKKALQEAHQQTLDDLQAEEDKVNTLTKAKTKLEQQVDDLEGSLEQEKKLRMDLERAKRKLEGDLKLAQESTMDIENDKQQLDEKLKKKEFEMTNLQSKIEDEQALAMQLQKKIKELQARIEELEEEIEAERASRAKAEKQRSDLSRELEEISERLEEAGGATSAQIEMNKKREAEFQKMRRDLEEATLQHEATAAALRKKHADSVAELGEQIDNLQRVKQKLEKEKSELKMEIDDLAGNMETVSKAKGNLEKMCRTLEDQLSEVKTKEEEHQRLINELSAQKARLHTESGEFSRQLDEKDAMVSQLSRGKQAFTQQIEELKRQLEEETKAKSALAHALQSSRHDCDLLREQYEEEQEAKAELQRAMSKANSEVSQWRTKYETDAIQRTEELEEAKKKLAQRLQDAEEHVEAVNSKCASLEKTKQRLQNEVEDLMIDVERSNAACAALDK

SEQ ID NO: 67 - protein, 667 aa, Oryctolagus cuniculus
TLDDLQAEEDKVNTLTKAKTKLEQQVDDLEGSLEQEKKLRMDLERAKRKLEGDLKLAQESTMDIENDKQQLDEKLKKKEFEMTNLQSKIEDEQALAMQLQKKIKELQARIEELEEEIEAERASRAKAEKQRSDLSRELEEISERLEEAGGATSAQIEMNKKREAEFQKMRRDLEEATLQHEATAAALRKKHADSVAELGEQIDNLQRVKQKLEKEKSELKMEIDDLAGNMETVSKAKGNLEKMCRTLEDQLSEVKTKEEEHQRLINELSAQKARLHTESGEFSRQLDEKDAMVSQLSRGKQAFTQQIEELKRQLEEETKAKSALAHALQSSRHDCDLLREQYEEEQEAKAELQRAMSKANSEVSQWRTKYETDAIQRTEELEEAKKKLAQRLQDAEEHVEAVNSKCASLEKTKQRLQNEVEDLMIDVERSNAACAALDKKQRNFDKVLAEWKHKYEETQAELEASQKESRSLSTEVFKVKNAYEESLDHLETLKRENKNLQQEISDLTEQIAESGKHIHELEKVKKQIDQEKSELQAALEEAEGSLEHEEGKILRIQLELNQVKSEIDRKIAEKDEEIDQLKRNHLRVVESMQSTLDAEIRSRNDALRIKKKMEGDLNEMEIQLNHANRQAAEAIKNLRNTQGILKDTQLHLDDAVRGQDDLKEQLA

SEQ ID NO: 68 - protein, 830 aa, Oryctolagus cuniculus
LAQESTMDIENDKQQLDEKLKKKEFEMTNLQSKIEDEQALAMQLQKKIKELQARIEELEEEIEAERASRAKAEKQRSDLSRELEEISERLEEAGGATSAQIEMNKKREAEFQKMRRDLEEATLQHEATAAALRKKHADSVAELGEQIDNLQRVKQKLEKEKSELKMEIDDLAGNMETVSKAKGNLEKMCRTLEDQLSEVKTKEEEHQRLINELSAQKARLHTESGEFSRQLDEKDAMVSQLSRGKQAFTQQIEELKRQLEEETKAKSALAHALQSSRHDCDLLREQYEEEQEAKAELQRAMSKANSEVSQWRTKYETDAIQRTEELEEAKKKLAQRLQDAEEHVEAVNSKCASLEKTKQRLQNEVEDLMIDVERSNAACAALDKKQRNFDKVLAEWKHKYEETQAELEASQKESRSLSTEVFKVKNAYEESLDHLETLKRENKNLQQEISDLTEQIAESGKHIHELEKVKKQIDQEKSELQAALEEAEGSLEHEEGKILRIQLELNQVKSEIDRKIAEKDEEIDQLKRNHLRVVESMQSTLDAEIRSRNDALRIKKKMEGDLNEMEIQLNHANRQAAEAIKNLRNTQGILKDTQLHLDDAVRGQDDLKEQLAMVERRANLMQAEIEELRASLEQTERSRRVAEQELLDASERVQLLHTQNTSLINTKKKLETDISQIQGEMEDIVQEARNAEEKAKKAITDAAMMAEELKKEQDTSAHLERMKKNMEQTVKDLQHRLDEAEQLALKGGKKQIQKLEARVRELENEVESEQKRNVEAVKGLRKHERRVKELTYQTEEDRKNVLRLQDLVDKLQSKVKAYKRQAEEAEEQSN

SEQ ID NO: 69 - protein, 1218 aa, Oryctolagus cuniculus
VFVADPKESFVKATVQSREGGKVTAKTEAGATVTVKEDQVFPMNPPKYDKIEDMAMMTHLHEPAVLYNLKERYAAWMIYTYSGLFCVTVNPYKWLPVYNAEVVTAYRGKKRQEAPPHIFSISDNAYQFMLTDRENQSILITGESGAGKTVNTKRVIQYFATIAVTGDKKKEEATSGKMQGTLEDQIISANPLLEAFGNAKTVRNDNSSRFGKFIRIHFGTTGKLASADIETYLLEKSRVTFQLKAERSYHIFYQIMSNKKPDLIEMLLITTNPYDYAFVSQGEITVPSIDDQEELMATDSAIDILGFTSDERVSIYKLTGAVMHYGNMKFKQKQREEQAEPDGTEVADKAAYLQSLNSADLLKALCYPRVKVGNEYVTKGQTVQQVYNAVGALAKAVYEKMFLWMVTRINQQLDTKQPRQYFIGVLDIAGFEIFDFNSLEQLCINFTNEKLQQFFNHHMFVLEQEEYKKEGIEWEFIDFGMDLAACIELIEKPMGIFSILEEECMFPKATDTSFKNKLYEQHLGKSNNFQKPKPAKGKVEAHFSLVHYAGTVDYNITGWLDKNKDPLNETVVGLYQKSAMKTLAFLFTGTAAAEAEGGGKKGGKKKGSSFQTVSALFRENLNKLMTNLRSTHPHFVRCIIPNETKTPGAMEHELVLHQLRCNGVLEGIRICRKGFPSRILYADFKQRYKVLNASAIPEGQFIDSKKASEKLLGSIDVDHTQYKFGHTKVFFKAGLLGLLEEMRDDKLAQLITRTQAMCRGFLARVEYKKMVERRESIFCIQYNIRAFMNVKHWPWMKLYFKIKPLLKSAETEKEMANMKEEFEKTKESLAKAEAKRKELEEKMVALMQEKNDLQLQVQAEADSLADAEERCDQLIKTKIQLEAKIKEVTERAEDEEEINAELTAKKRKLEDECSELKKDIDDLELTLAKVEKEKHATENKVKNLTEEMAGLDETIAKLTKEKKALQEAHQQTLDDLQAEEDKVNTLTKAKTKLEQQVDDLEGSLEQEKKIRMDLERAKRKLEGDLKLAQESTMDIENDKQQLDEKLKKKEFEMSNLQSKIEDEQALAMQLQKKIKELQARIEELEEEIEAERASRAKAEKQRSDLSRELEEISERLEEAGGATSAQIEMNKKREAEFQKMRRDLEEATLQHEATAATLRKKHADSVAELGEQIDNLQRVKQKLEKEKSELKMEIDDLASNMETVSKAKGNLEKMCRTL

SEQ ID NO: 70 - protein, 1574 aa, Oryctolagus cuniculus
TLVSDVDYRNYLHQWTCMPDQNDVIQAKKAYELQSDAVYKADLEWLRGIGWMPSDSVSVNHAKHAADIFSEKKYRTKIETLNFTPVDDRVDYVTAKQSGEILDDIKYRKDWNSIKSKYTLTETPLLHTVQEAARILDQYLYKESWEKQKATGYILPPDAVPFVHAHHSSDVQSELKYKAEHVKQRGHYVGVPTMRDDPKLVWFEHAGQIQNDRLYKEDYHKTKAKINIPADMVSVVAAKEGQNLVSDIDYRNYLHQWTCHPDQNDVIQARKAYDLQSDNVYKADLEWLRGIGWIPLDSVDHVRVTKNQEMVNQIKYKKDALDNYPNFTSVVDPPAIVLAKINAVNQSDVKYKETFNKAKGKYTFSPDTPHISHSKDMGKLYSTILYKGAWEGTKAYGYTLDERYIPIVGAKHADFVNSELKYKETYEKQKGHYLAGKVISEFPGVVHCLDFQKMRSVLNYRRDYENTKANVHIPNDMMNHVLAKKCQYILSDLEYRHYFHQWTSLPDEPKVLLARNAQEILSDNVYKDDLNWLKGIGCYVWDTPQILHAKKSYDLQSQLQYTAAGKENLQNYNLVTDTPLYVTALQSGINASEVKYKENYHQIKDKYTTVLETVDYDRTRNLKDLYSSNLYKEAWNKVKATSYILPTSTLSLTHAKNQKHLASHIKYREEYEKFKALYTLPKSVEDDPNTARCLRVGKLNIDRLYRSVYEKNKMKIHIVPDMVEMVTAKDTQKKVSEIDYRLHLHEWICHPDLQVNSHVRKVTDQISDIVYKDDLNWLKGIGCYVWDTPEILHAKHAYDLRNDIKYKAHMMKTRNDYKLVTDTPVYVQAVKSGKQLSDAVYHHDYVHSVRGKVAPTTKTVDLERALHAYKLQSENLYKTSLRTLPTGYRLPGDTPHFKHNKDIRYMSSYFKYKEVYEHIKANGYTLGPNDVPFVNVRRVNKVTSERLYRELYHKLKDKIHTTPDTPEIRQVKKTQEAVSELIYKSDFFKMQGHMISLPYTPHVLHCRYVGDITSDIKYKEDLQILKGLGCFLYDTPDMVRSRHLRKLWSNYLYTDNARKMRDKYKVVLDTPEYRKVQELKTHLSELVYRASGRKQKSIFTSVPDTPDLLRAKRGQKLQSQYLYVELATKERPHHHAGNQTTALKHARDVKDMVSEKKYKVQYEKMKDKYTPIPDTPILIRAKNAYWNASDLRYKETFQKTKGKYHTVKDALDIVYHRKVTDDISKVKYKENYMRHLGIWRSIPDRPEHFHHRAVTDAVSDIKYKEDLTWLKGIGCYAYDTPDFTLAEKNKTLYSKYKYKEVFERTKSDFKYVADCPINRHFKYATQLMNEKKYRADYEQRKDKYQLVVDEPRHLLAKTAGDQISQIKYRKQYEKSKDKFTSIVDTPEHLRTTKVNKQISDILYKLEYNKAKPRGYTTIHDTPMLLHVRKVKDEVSDLKYKEVYQRNKSNCTIKPDAVHIKAAKDAYKVNTNLDYKKQYEANKAHWRWTPDRPDFIQAAKSSLQQSDFEYKLDREFLKGCKLSVTDDKDMVLALKNSLIESDLKYKEKHVKERGSCHAVPDTPQILLAKTVSNLV

SEQ ID NO: 71 - protein, 1500 aa, Oryctolagus cuniculus
EHTRNVNQIQSDNVYKDEYNNFFKGIGWIPIGSLEVEKVKKAGDALNEKKYRQHPDTIKFTSVPDSMGMVLAQHNTKQLSDLNYKVEGEKVKHKYTMDPEVPQFIQARVNAFNLSDAHYKADWKKTLAKGYDLRPDAIPIVAAKSSRNIASDCKYKEAYEKAKGKQIGFLSLQDDPKLVHYMNVAKIQSDREYRKGYEASKTRYHTPLDMLSVTAAKKSQEVATNANYKQPFHNYTLLPDALPVEHSRNAMQIQSDNLYKSDFTGWMKGIGWIPIQSLEVEKAKKAGEILSEKRYRQHPEKLKFTYAMDTMEQALNKANKLNMDKRLYTKKWDKDKTTIHVMPDTPDILLSRVNQITMSDKLYRAGWEEEKKKGYDLRPDAISIKAAKASRDIASDYKYKQAYEQAKGKHIGFRSLEDDPKLVHFMQVAKMQSDREYKKAYEKSKTSFHTPVDMLSVVAAKKSQEVATNANYRNVIHTYNMLPDAMGFELAKNMMQIQSDNQYKADYADFMKGIGWLPLGSLEAEKNKKAMEIISEKKYRQHPDTLKYSTLMDSMNMVLAQNNAKIMNEHLYKQAWEADKTKVHIMPDIPQIVLAKANAINMSDKLYKLSMEEAKKKGYDLRTDAIPIKAAKASRDIASDYKYKYNYEKQKGKMVGFRSLEDDPKLVHSMQVAKMQSDREYKKNYEKTKTSYHTPADMLSVTAAKDAQANITNTNYKHLIHKYILLPDAMSLELSRNMNRIQSDNEYKQDYNEWYKGLGWSPAGSLDVEKAKKATEYASDQKYRQHPSNFQFKKLTDSMDMVLAKQNAHTMNKHLYTIDWNKDKTKIHVMPDTPDILQAKQNQTLFSQKFYKLGWEEALKKGYDLPVDAISVQLAKASRDIASDFKYKQGYRKQLGHHVGFRSLQDDPKLVLSMNVAKMQSEREYKKDFEKWKTKFSSPVDMLGVVLAKKCQALVSDVDYKNYLHQWTCLPDQNDVIHAKKAYELQSENMYKSDLEWLKGIGWSPLGSLEAEKNKRASEILSEKKYRQPPDTNKFTSIPDAMDIVLAKTNAKNRSDRLYREAWDKDKTQVHIMPDTPDIILAKANLINTSDKLYRMGYEELKKKGYDLPVDAIPIKAAKASREIASEYKYKEGFRKQLGHHIGARNIKDDPKMMWSMHVAKIQSDREYKKDFEKWKTKFSSPVDMLGVVLAKKCQTLVSDIDYKNYLHQWTCLPDQNDVTHARHAYDLQSDNLYKSDLQWLKGIGWIPTGSIEDEKNKRATQILSDHVYRQHPDKFKFSILMDSIPMVLAKNNATNMNHRLYTEAWDKDKTTVHIMPDTPEVLLAKQNQVNCSEKLYKLGLEETKRKGYDMRVDAIPIKAAKASRDIASEFKYKEGYRKQLGHHIGARAIHDDPKMMWSMHVAKIQSDREYKKDFEKWKTKFSSPVDMLGVVLAKKCQALVSDVDYKNYLHQWTCLPDQNDVMHARQAYDLQSDNLYKSDLQWLRGIGWVPIGSLDVEKC

SEQ ID NO: 72 - protein, 1400 aa, Oryctolagus cuniculus
DVTHARHAYDLQSDNLYKSDLQWLKGIGWIPTGSIEDEKNKRATQILSDHVYRQHPDKFKFSILMDSIPMVLAKNNATNMNHRLYTEAWDKDKTTVHIMPDTPEVLLAKQNQVNCSEKLYKLGLEETKRKGYDMRVDAIPIKAAKASRDIASEFKYKEGYRKQLGHHIGARAIHDDPKMMWSMHVAKIQSDREYKKDFEKWKTKFSSPVDMLGVVLAKKCQALVSDVDYKNYLHQWTCLPDQNDVMHARQAYDLQSDNLYKSDLQWLRGIGWVPIGSLDVEKCKKATEILSDKIYRQPPDRFKFTSVTDSLEQVLAKNNAINMNKRLYTEAWDKDKTQIHIMPDTPEIMLARMNKINYSESLYKLANEEAKKKGYDLRSDAIPIVAAKASRDIASDYKYKDGYRKQLGHHIGARDIKDDPKMMWSMHVAKIQSDREYKKDFEKWKTKFSSPVDMLGVVLAKKCQTLVSDIDYKNYLHQWTCLPDQNDVMHARQAYDLQSDNIYKSDLQWLRGIGWVPIGSLEVAKCKRAAEILSDNIYRQPPDKLKFTSVPDSLEQVLAKNNAINMNKRLYTEAWDKDKTQIHIMPDTPEIMLARQNKINYSESLYKLANEEAKKKGYDLRSDAIPIVAAKASRDIASDYKYKDGYRRQLGHHIGARDIKDDPKMMWSMHVAKIQSDREYKKDFEKWKTKFSSPVDMLGVVLAKKCQTLVSDIDYKNYLHQWTCLPDQNDVMHARQAYDLQSDNIYKSDLQWLRGIGWVPIGSVDLVKCKRAAEILSDNIYRQSPDKLKFTSVTDSLEQVLAKNNAINMNKRLYTEAWDKDKTQIHMMPDTPEIMLARQNKMNYSENFYRQAMEEAKKEGYDLRSDAIPIVAAKASRDIASDYKYKEAYRKQLGHHIGARAIHDDPKMMWSLHIAKVQSDREYKKEFEKYKTRYSSPVDMLGIVLAKKCQTLVSDVDYKNLLHEWTCLPDQNDVIQARKAYDLQSDNLYKSDLEWLRGIGWVPIGSLEVVKAKRATEILSDNLYRTRPDTLKFTSVTDSLEQVLAKNNAINMNKRLYTEAWENDKKTIHVMPDTPEIMLAKLNKINYSDKLYKLALEESKKEGYDLRVDAIPIQAAKASRDIASDYKYKEGYRKQLGHHIGARNIKDDPKMMWSMHVAKIQSDREYKKDFEKWKTKFSSPVDMLGVVLAKKCQILVSDIDYKHPLHEWICLPDQNDVIQARKAYDLQSDAVYKADLEWLRGIGWVPIGSVEVEKVKRAGEILSDRKYRQPADKLKFTSITDTPEIVLAKSNALTMSKHLYTEAWDADKTSIHVMPDTPEILLAKSNSANISQKLYTKGWDESKMKDYDLKADAIPIKSAKASRDIASDYKYKEAYEKQKGHHIGAQSIEDDPKIMCAIHA

SEQ ID NO: 73 - protein, 1500 aa, Oryctolagus cuniculus
DKRETSRPNWAQVSANVPITSCSVEKLIEGHEYQFRICAENKYGVGDPVFTEPAIAKNPYDPPGRCDPPVISNVTKDHMTVSWKPPADDGGSPITGYLLEKRETHAVNWTKVNRKPVIERTIKATGLQEGTEYEFRVTAINKAGPGKPSDASKAVYAQDPLYPPGPPAFPKVYDTTRSSVSLSWGKPAYDGGSPIIGYLVEVKRADTDNWVRCNLPQKLQKTRFEVTGLMENTEYQFRVYAVNKVGYSDPSDVPDKHCPKDILIPPEGELDADLRKTLILRAGVTMRLYVPVKGRPPPKITWSKPNVNLRERIGLDIKSTDFDTFLRCENVNKYDAGKYILTLENSCGKKEYTIVVKVLDTPGPPVNVTVKEISRDSAYITWDPPIVDGGSPIINYVVEKRDAERKSWSTVTTECSKTSFRVSNLEEGKSYFFRVFAENEYGIGDPGETRDAVKASETPGPVVDLKVLTVTKSSCNIGWKKPRSDGGSRITGYVVDFLTEENKWQRVMKSLSLQYSTKDLNEGKQYTFRVSAENENGEGTPSEITVVAKDDVVAPDLDLKDLPDLCYLAKENSNFRLKIPIKGKPAPSVTWKKGEDPLATDTRVSVESSAVNTTLVVYDCQKSDAGKYTITLKNVAGTKEGTLSIKVVGKPGIPTGPIKFDEVTAEAITLKWGPPKDDGGSEITNYILEKRDSVNNKWVTCASAVQKTTFRVTRLHEGMEYTFRVSAENKYGVGEGLKSEPIVAKHPFDVPDAPPPPNIVDVRHDSVSLTWTDPRKTGGSPITGYHIEFKERNSLLWKRANKTPIRMKDFKVTGLTEGLEYEFRVMAINLAGVGKPSLPSEPVVALDPIDPPGKPEVINVTRNSVTLIWTEPKYDGGHKLTGYIVEKRDLPSKTWMKANHINVPDCAFTVTDLVEGGKYEFRIRAKNTAGAISAPSESTGTIICKDEYEAPTIVLDPTIKDGLTIKAGDTIVLNAISILGKPLPKSSWSKAGKDIRPSDITQITSTPTSSMLTVKYASRKDAGEYTITATNPFGTKEEHVRVTVLDVPGPPGPIEISNVSAEKATLTWTPPLEDGGSPIKSYVLEKRETSRLLWTVVAEDIQSCRHVVTKLIQGNEYLFRVSAVNHYGKGEPVQSEPVKMVDRFGPPGPPGKPEVSNVTKNTATVSWKRPTDDGGSEITGYYVERREKKGLRWVRATKTPVSDLRCKVTGLQEGNTYEFRVSAENRAGIGPPSDASNYVLMKDVAYPPGPPSNARVTDTTKKSASLAWGKPHYDGGLEITGYVVEHQKVGDETWVKDTTGPALRITEFVVPDLHTKEKYNFRISAINDAGVGEPAVIPDVEIVEREMAPDFELDAELRRTLVVRAGLSIRIFVPIKGRPAPEVTWTKDDINLKTRANIENTESFTLLIIPECNRYDTGKFVMTIENPAGKKSGFVNVRVLDTPGPVLNLRPTDITKDSVTLHWDLPLIDGGSRITNYIVEKREATRKSYSTVTTKCHKCT

SEQ ID NO: 74 - protein, 338 aa, Oryctolagus cuniculus
DKETPSGFTLDDVIQTGVDNPGHPFIMTVGCVAGDEESYTVFKDLFDPIIQDRHGGFKPTDKHKTDLNHENLKGGDDLDPHYVLSSRVRTGRSIKGYTLPPHCSRGERRAVEKLSVEALNSLTGEFKGKYYPLKSMTEQEQQQLIDDHFLFDKPVSPLLLASGMARDWPDARGIWHNDNKSFLVWVNEEDHLRVISMEKGGNMKEVFRRFCVGLQKIEEIFKKAGHPFMWNEHLGYVLTCPSNLGTGLRGGVHVKLAHLSKHPKFEEILTRLRLQKRGTGGVDTAAVGSVFDISNADRLGSSEVEQVQLVVDGVKLMVEMEKKLEKGQSIDDMIPAQK

SEQ ID NO: 75 - protein, 808 aa, Oryctolagus cuniculus
GGGGGGEYMEQEEDWDRDLLLDPAWEKQQRKTFTAWCNSHLRKAGTQIENIEEDFRNGLKLMLLLEVISGERLPRPDKGKMRFHKIANVNKALDFIASKGVKLVSIGAEEIVDGNLKMTLGMIWTIILRFAIQDISVEETSAKEGLLLWCQRKTAPYRNVNVQNFHTSWKDGLALCALIHRHRPDLIDYAKLRKDDPIGNLNTAFEVAEKYLDIPKMLDAEDIVNTPKPDEKAIMTYVSCFYHAFAGAEQAETAANRICKVLAVNQENEKLMEEYEKLASELLEWIRRTVPWLENRVGEPSMSAMQRKLEDFRDYRRLHKPPRVQEKCQLEINFNTLQTKLRLSHRPAFMPSEGKLVSDIANAWRGLEQVEKGYEDWLLSEIRRLQRLQHLAEKFQQKASLHETWTRGKEDMLTQRDYESASLQEVRALLRRHEAFESDLAAHQDRVEHIAALAQELNELDYHEAASVNSRCQAICDQWDNLGTLTQKRRDALERMEKLLETIDQLQLEFARRAAPFNNWLDGAIEDLQDVWLVHSVEETQSLLTAHDQFKATLPEADRERGAILGIQGEIQKICQTYGLRPSSTNPYINLSPQDINTKWDTVRKLVPSRDQTLQEELARQQMNERLRRQFAAQANAVGPWIQAKVEEVGRLAAGLAGSLEEQMAGLRQQEQNIINYKSNIDRLEGDHQLLQESLVFDNKHTVYSMEHIRVGWEQLLTSIARTINEVENQVLTRDAKGLSQEQLNEFRASFNHFDRKRNGMMEPDDFRACLISMYDLGEVEFARIMTMVDPNAAGVVTFQAFIDFMTR

SEQ ID NO: 76 - protein, 761 aa, Oryctolagus cuniculus
IYYLSLEFYMGRTLQNTMVNLALENACDEATYQLGLDMEELEEIEEDAGLGNGGLGRLAACFLDSMATLGLAAYGYGIRYEFGIFNQKICGGWQMEEADDWLRYGNPWEKARPEFTLPVHFYGRVEHTSQGAKWVDTQVVLAMPYDTPVPGYRNNVVNTMRLWSAKAPNDFNLKDFNVGGYIQAVLDRNLAENISRVLYPNDNFFEGKELRLKQEYFVVAATLQDIIRRFKSSKFGCRDPVRTNFDAFPDKVAIQLNDTHPSLAIPELMRVLVDLERLDWDKAWEVTVKTCAYTNHTVLPEALERWPVHLLETLLPRHLQIIYEINQRFLNRVAAAFPGDVDRLRRMSLVEEGAVKRINMAHLCIAGSHAVNGVARIHSEILKKTIFKDFYELEPHKFQNKTNGITPRRWLVLCNPGLAEIIAERIGEEYISDLDQLRKLLSYVDDEAFIRDVAKVKQENKLKFAAYLEREYKVHINPNSLFDVQVKRIHEYKRQLLNCLHVITLYNRIKKEPNKFVVPRTVMIGGKAAPGYHMAKMIIKLITAIGDVVNHDPVVGDRLRVIFLENYRVSLAEKVIPAADLSEQISTAGTEASGTGNMKFMLNGALTIGTMDGANVEMAEEAGEENFFIFGMRVEDVDRLDQRGYNAQEYYDRIPELRQIIEQLSSGFFSPKQPDLFKDIVNMLMHHDRFKVFADYEEYVKCQERVSALYKNPREWTRMVIRNIATSGKFSSDRTIAQYAREIWGVEPSRQRLPAPDEKIP

SEQ ID NO: 77 - protein, 351 aa, Oryctolagus cuniculus
SHPALTPEQKKELSDIAHRIVAPGKGILAADESTGSIAKRLQSIGTENTEENRRFYRQLLLTADDRVNPCIGGVILFHETLYQKADDGRPFPQVIKSKGGVVGIKVDKGVVPLAGTNGETTTQGLDGLSERCAQYKKDGADFAKWRCVLKIGEHTPSALAIMENANVLARYASICQQNGIVPIVEPEILPDGDHDLKRCQYVTEKVLAAVYKALSDHHIYLEGTLLKPNMVTPGHACTQKYSHEEIAMATVTALRRTVPPAVTGVTFLSGGQSEEEASINLNAINKCPLLKPWALTFSYGRALQASALKAWGGKKENLKAAQEEYVKRALANSLACQGKYTPSGQAGAAAS

SEQ ID NO: 78 - protein, 584 aa, Oryctolagus cuniculus
EAHKSEIAHRFNDVGEEHFIGLVLITFSQYLQKCPYEEHAKLVKEVTDLAKACVADESAANCDKSLHDIFGDKICALPSLRDTYGDVADCCEKKEPERNECFLHHKDDKPDLPPFARPEADVLCKAFHDDEKAFFGHYLYEVARRHPYFYAPELLYYAQKYKAILTECCEAADKGACLTPKLDALEGKSLISAAQERLRCASIQKFGDRAYKAWALVRLSQRFPKADFTDISKIVTDLTKVHKECCHGDLLECADDRADLAKYMCEHQETISSHLKECCDKPILEKAHCIYGLHNDETPAGLPAVAEEFVEDKDVCKNYEEAKDLFLGKFLYEYSRRHPDYSVVLLLRLGKAYEATLKKCCATDDPHACYAKVLDEFQPLVDEPKNLVKQNCELYEQLGDYNFQNALLVRYTKKVPQVSTPTLVEISRSLGKVGSKCCKHPEAERLPCVEDYLSVVLNRLCVLHEKTPVSEKVTKCCSESLVDRRPCFSALGPDETYVPKEFNAETFTFHADICTLPETERKIKKQTALVELVKHKPHATNDQLKTVVGEFTALLDKCCSAEDKEACFAVEGPKLVESSKATLG

SEQ ID NO: 79 - protein, 381 aa, Bos taurus
MPFGNTHNKHKLNFKAEEEYPDLSKHNNHMAKALTLEIYKKLRDKETPSGFTLDDVIQTGVDNPGHPFIMTVGCVAGDEESYTVFKDLFDPIIQDRHGGFKPTDKHKTDLNHENLKGGDDLDPNYVLSSRVRTGRSIKGYALPPHCSRGERRAVEKLSVEALNSLTGEFKGKYYPLKSMTEQEQQQLIDDHFLFDKPVSPLLLASGMARDWPDARGIWHNDNKSFLVWVNEEDHLRVISMEKGGNMKEVFRRFCVGLQKIEEIFKKAGHPFMWNEHLGYVLTCPSNLGTGLRGGVHVKLAHLSKHPKFEEILTRLRLQKRGTGGVDTAAVGSVFDVSNADRLGSSEVEQVQLVVDGVKLMVEMEKKLEKGQSIDDMIPAQK

SEQ ID NO: 80 - protein, 901 aa, Bos taurus
MMMVLQPEGLGTGEGPFAGGRGGGEYMEQEEDWDRDLLLDPAWEKQQRKTFTAWCNSHLRKAGTQIENIEEDFRNGLKLMLLLEVISGERLPRPDKGKMRFHKIANVNKALDFIASKGVKLVSIGAEEIVDGNLKMTLGMIWTIILRFAIQDISVEETSAKEGLLLWCQRKTAPYRNVNVQNFHTSWKDGLALCALIHRHRPDLIDYAKLRKDDPIGNLNTAFEVAEKYLDIPKMLDAEDIVNTPKPDEKAIMTYVSCFYHAFAGAEQAETAANRICKVLAVNQENEKLMEEYEKLASELLEWIRRTVPWLENRVGEPSMSAMQRKLEDFRDYRRLHKPPRVQEKCQLEINFNTLQTKLRLSHRPAFMPSEGKLVSDIANAWRGLEQAEKGYEDWLLSEIRRLQRLQHLAEKFQQKASLHEAWTRGKEDMLSQRDYETASLQEVRALLRRHEAFESDLAAHQDRVEHIAALAQELNELDYHEAASVNSRCQAICDQWDNLGTLTQKRRDALERMEKLLETIDQLQLEFARRAAPFNNWLDGAVEDLQDVWLVHSVEETQSLVTAHDQFKATLPEADRERGAILGIQGEIQKICQTYGLRPSSTNPYITLTPQDINTKWDTVRKLVPSRDQMLQEELTRQQVNERLRRQFAAQANAIGPWIQGKVEEVGRLAAGMAGSLEEQMAGLRQQEQNIINYKSNIDRLEGDHQLLQESLVFDNKHTVYSMEHIRVGWEQLLTSIARTINEVENQVLTRDAKGLSQEQLNEFRASFNHFDRKRNGMMEPDDFRACLISMGYDLGEVEFARIMTMVDPNAAGVVTFQAFIDFMTRETAETDTAEQVVASFKILAGDKNYITAEELRRELPAEQAEYCIRRMAPYKGAGAPAGALDYVAFSSALYGESDL

SEQ ID NO: 81 - protein, 842 aa, Bos taurus
MSRPLTDQEKRKQISVRGLAGVENVTELKKNFNRHLHFTLVKDRNVATPRDYYFALAYTVRDHLVGRWIRTQQHYYEKDPKRIYYLSLEFYIGRTLQNTMVNLALENACDEATYQLGLDMEELEEIEEDAGLGNGGLGRLAACFLDSMATLGLAAYGYGIRYEFGIFNQKISGGWQMEEADDWLRYGNPWEKARPEFTLPVHFYGRVEHTSQGAKWVDTQVVLAMPYDTPVPGYRNNVVNTMRLWSAKAPNDFNLKDFNVGGYIQAVLDRNLAENISRVLYPNDNFFEGKELRLKQEYFVVAATLQDIIRRFKSSKFGCLDPVRTNFDAFPDKVAIQLNDTHPSLAIPELMRILVDQERLEWEKAWEVTVKTCAYTNHTVLPEALERWPVHLIETLLPRHLQIIYEINQRFLNRVAAAFPGDVDRLRRMSLVEEGAVKRINMAHLCIAGSHAVNGVARIHSEILKKTIFKDFYELEPHKFQNKTNGITPRRWLVMCNPGLAEIIAERIGEEYIADLDQLRKLLSYVDDESFIRDVAKVKQENKLKFSAYLEKEYKVHINPNSLFDIQVKRIHEYKRQLLNCLHVITLYNRIKKEPNKFFVPRTVMIGGKAAPGYHMAKMIIKLITAIGDVVNHDPVVGDRLRVIFLENYRVSLAEKVIPAADLSEQISTAGTEASGTGNMKFMLNGALTIGTMDGANVEMAEEAGEENFFIFGMRVEDVERLDQKGYNAQEYYDRIPELRHVIDQLSSGFFSPKQPDLFKDIVNMLMHHDRFKVFADYEEYIKCQERVSALYKNPREWTRMVIRNIATSGKFSSDRTIAQYAREIWGVEPTRQRMPAPDEKI

SEQ ID NO: 82 - protein, 364 aa, Bos taurus
MPHQYPALTPEQKKELCDIAHRIVAPGKGILAADESTGSIAKRLQSIGTENTEENRRFYRQLLLTADDRVNPCIGGVILFHETLYQKADDGRPFPQVIKAKGGVVGIKVDKGVVPLAGTNGETTTQGLDGLSERCAQYKKDGADFAKWRCVLKIGEHTPSSLAIMENANVLARYASICQQNGIVPIVEPEILPDGDHDLKRCQYVTEKVLAAVYKALSDHHIYLEGTLLKPNMVTPGHACTQKYSHEEIAMATVTALRRTVPPAVPGITFLSGGQSEEEASINLNAINKCPLLKPWALTFSYGRALQASALKAWGGKKENLKAAQEEYVKRALANSLACQGKYTPSGKAGAAASESLFISNHAY

SEQ ID NO: 83 - protein, 192 aa, Bos taurus
MAPKKDVKKPAAAAAPAPAPAPAPAPAPAPPKEEKIDLSAIKIEFSKQQQDEFKEAFLLFDRTGECKITLSQVGDVLRALGTNPTNAEVKKVLGNPSNEEMNAKKIEFEQFLPMLQAISNNKDQGTYEDFVEGLRVFDKEGNGTVMGAELRHVLATLGEKMKEEEVEALMAGQEDSNGCINYEAFVKHIMSN

SEQ ID NO: 84 - protein, 333 aa, Bos taurus
MVKVGVNGFGRIGRLVTRAAFNSGKVDIVAINDPFIDLHYMVYMFQYDSTHGKFNGTVKAENGKLVINGKAITIFQERDPANIKWGDAGAEYVVESTGVFTTMEKAGAHLKGGAKRVIISAPSADAPMFVMGVNHEKYNNTLKIVSNASCTTNCLAPLAKVIHDHFGIVEGLMTTVHAITATQKTVDGPSGKLWRDGRGAAQNIIPASTGAAKAVGKVIPELNGKLTGMAFRVPTPNVSVVDLTCRLEKPAKYDEIKKVVKQASEGPLKGILGYTEDQVVSCDFNSDTHSSTFDAGAGIALNDHFVKLISWYDNEFGYSNRVVDLMVHMASKE

SEQ ID NO: 85 - protein, 284 aa, Bos taurus
MDAIKKKMQMLKLDKENALDRAEQAEADKKAAEDRSKQLEDELVSLQKKLKATEDELDKYSEALKDAQEKLELAEKKATDAEADVASLNRRIQLVEEELDRAQERLATALQKLEEAEKAADESERGMKVIESRAQKDEEKMEIQEIQLKEAKHIAEDADRKYEEVARKLVIIESDLERAEERAELSEGKCAELEEELKTVTNNLKSLEAQAEKYSQKEDKYEEEIKVLSDKLKEAETRAEFAERSVTKLEKSIDDLEDELYAQKLKYKAISEELDHALNDMTSI

SEQ ID NO: 86 - protein, 1141 aa, Bos taurus
MPEAKPAAKKAPAGKDAAAKPAPKEATPQEAPAAPPTEAPPEDQSPTAEEPTGVFLKKPDSVSVETGKDTVIVAKLNGKELPAKPAVKWFKGKWLELGSKSGARFSFKESHDAASNVYTIELHITKVVLGDRGDYRIEVKAKDFCDSCAFNIDVEAPRQDSAGQSLESFKRSGEAKSDTAGELDFSGLLKKRQVVEEEKKKKKDDDDLGIPPEIWELLKGAKKSEYERIAFQYGITDLRGMLKRLKKAKVEVKKSAAFTKKLDPAYQVDRGNKIKLVVEISDPDLPLKWFKNGQEIKPSSKYVFENVGKKRILTINKCTLADDAAYEVVVKDEKCFTELFVKEPPVLIVTPLEDQQVFVGDRVEMAVEVSEEGAQVMWLKDGVELTREDSFKARYRLKKDGKRHILIYSEVTMEDKGHYQVMTNGGQCEAELIVEEKQLEVLQDIADLTVKASEQAVFKCEVSDEKVTGKWYKNGVEVRPSKRITISHTGRFHKLVIDDVRPEDEGDYTFVPDGYALSLSAKLKFLEIKVEYVPKQEPPKIHLDCSGQTSENAIVVVAGNKLRMDVSITGEPRPVATWMKEDEVFTGTEGRVRIEQRGDISSFVIESAERGDEGRYTIKVTNPVGEDVASILLKVVDVPDPPEAVRVTSVGEDWAILVWEPPKYDGGRPVTGYLLERKKKGSQRWMKLNFEVFTETTYESTNMIEGILYEMRVFAVNVIGVSQPSMNTKPFMPIAPTSEPQHLTVEDVTDTTTTLKWRPPDKIGAGGIDGYLIEYCVEGSDEWIPANTEPVERCGFTVKNLPTGAKITFRVVGVNIAGRSQPATLAQPVTIREIVEQPKIRLPRHLRQTYVRKVGEHLNLVIPFQGKPRPQVVWTKGGAPIDTSHVHVRTSDFDTVFFVRQAARSDSGEYELTVQIENMKDTATVCIRVVEKAGPPQNVMVKEVWGTNALVEWQPPKDDGNSEITGYFVQKADKKTMEWFTVYERNRHTSCTVSDLIMGNEYYFRVYSENVCGLSDLPGVSKNTARIVKTGMTLKLPEYKEHDFRTPPKFLTPLPDRVVVAGYAAALNCAVRGHPKPKVVWMKNKMEIREDPKFLMTNQQGVLTLNIRRPSPFDSGTYSCRAVNELGEALAECKLEVRVPQ

SEQ ID NO: 87 - protein, 1495 aa, Bos taurus
MSLVAVPFYQKRHKHFDQSYRNIQTRYLLDEYSAKKKRASAQSSLQRSVTRKSSSQRESSRAALGESTLGETTLGETSLGDTTLGRTTCRLCAKRMSSALEEEAQERKRRYQSMVAAYGEAKRERYLSELAQLEEDVHVARTHARDQLDRLALQHAGRHGLHHAVDDRLAWERHSFEERISRAPEILVRLRSHTVWERMSVKLCFTVQGFPTPVVQWYKDGSLICQAGEPGKYRIDSKYGVHTLEINRADFDDTATYSAVATNVHGQVSTNAAVVVRRFRGDEEPFHSVGLPIGLPLSSVIPYTHFDVQFIEKFGVTFRREGETLTLKCTLLVTPDLKRVQPRAEWYRDDVLLKESKWTKMFFGEGQASLSFSHLNKDDEGLYTLRIVSRGGITDHSAFLFVRDADPLVTGAPGAPMDVHCHDANRDYVIVTWKPPNATKESPVIGYFIDRCEVGTDNWIQCNDAPVKICKYPVTGLFEGRSYIFRVRAVNSAGISRPSRVSEAVAALDPVDLRRLQAVHLEGEKDIVVYQEELEGEVQIPGPPTNVHASETSRTYVVLSWDPPEPRGKEPLMYFIEKSMVGSGSWQRVNAQTAVRSPRYAVFDLAEGKPYVFRVLSANKHGLSDPSEITAPIQAQDTIVVPSAPGRVLASRNTRTSVVVQWDRPKHEEDLLGYYVDCSVAGSNVWEPCNHKPIGYNRFVVHGLTTGEQYIFRVKAVNAVGTSENSQESDVIKVQAALTVPSHPYGITLLNCDGHSMILGWKVPKFSGGSPILGYYVDKREAHHKNWHEVNSSPLKERILTVEGLTEGSLYEFKIAAANMAGIGQPSDPSELFKCEAWTMPEPGPAYDLTFCEVRDTSLVLLWKPPVYSGSSPVSGYFVDYKEEDSGEWLTVHETATPHRYLKVCDLHQGKTYVFRVRAVNASGVGRPSDTSEPVLVEARPGTKEVSAGVDEEGNVYLSFDCPEVTDASHFTWCKSYEDIADDDRFKVETAGDHSKLYFKNLDKEDLGTYSVSVSDTDGVSSSFVLDEEELERLKALSNEIKNPTIPLKSELAYEIFDKGQVRFWLQAEHLSPDSSYRFIINDREVADSETHRIKCDKSTGIIEMVMDRFTIDNEGTYTVQIQDGKAKSQSSLVLIGDAFKAVLKEAEFQRKEFLRKQGPHFAEYLHWDVTEECEVRLVCKVANTKKETVFKWLKDDVLYETEKMPDLEKGVCELLIPKLSKKDHGEYKATLKDDRGQDVSTLEIAGKVYEDMILAMSRVCGASASPLKILCTPEGIRLQCFMKYFTEEMKVNWYHKDAKITSSEHMRIGGNEQMAWLQICEPTEKDKGKYTFEILDGKKNHQRSLDLSGQAFDEAFAEFQQLKAAAFAEKNRGKVIGGLPDVVTIMEGKTLNLTCTVFGNPDPEVVWFKNDKDIELSDHFSVKVEQGKYVSMTIKGVTSEDSGKYSIHVKNKYGGEKIDVTVSVYKHGEEIPDVVLPQQAKPKLLPAAAPSPAH

SEQ ID NO: 88 - protein, 434 aa, Bos taurus
MAMQKIFAREILDSRGNPTVEVDLHTAKGRFRAAVPSGASTGIYEALELRDGDKSRYLGKGVLKAVEHINKTLGPALLEKKLSVVDQEKVDKFMIELDGTENKSKFGANAILGVSLAVCKAGAAEKGVPLYRHIADLAGNPELILPVPAFNVINGGSHAGNKLAMQEFMILPVGASSFREAMRIGAEVYHHLKGVIKAKYGKDATNVGDEGGFAPNILENNEALELLKTAIQAAGYPDKVVIGMDVAASEFYRNGKYDLDFKSPDDPARHISGEKLGELYKNFIKNYPVVSIEDPFDQDDWATWTSFLSGVNIQIVGDDLTVTNPKRIAQAVEKKACNCLLLKVNQIGSVTESIQACKLAQSNGWGVMVSHRSGETEDTFIADLVVGLCTGQIKTGAPCRSERLAKYNQLMRIEEALGDKAVFAGRKFRNPKAK

SEQ ID NO: 89 - protein, 170 aa, Bos taurus
MAPKKAKRRAAAEGGSSSVFSMFDQTQIQEFKEAFTVIDQNRDGIIDKEDLRDTFAAMGRLNVKNEELDAMMKEASGPINFTVFLNMFGEKLKGADPEDVITGAFKVLDPEGKGTIKKKFLEELLTTQCDRFSQEEIKNMWAAFPPDVGGNVDYKNICYVITHGDAKDQE

SEQ ID NO: 90 - protein, 1938 aa, Bos taurus
MSSDQEMAVFGEAAPYLRKSEKERIEAQNKPFDAKTSVFVADPKESFVKATVQSREGGKVTAKTEAGATVTVKEDQVFPMNPPKFDKIEDMAMMTHLHEPAVLYNLKERYAAWMIYTYSGLFCVTVNPYKWLPVYNAEVVTAYRGKKRQEAPPHIFSISDNAYQFMLTDRENQSILITGESGAGKTVNTKRVIQYFATIAVTGEKKKEEPTSGKMQGTLEDQIISANPLLEAFGNAKTVRNDNSSRFGKFIRIHFGTTGKLASADIETYLLEKSRVTFQLKAERSYHIFYQIMSNKKPELIEMLLITTNPYDYAYVSQGEITVPSIDDQEELMATDSAIEILGFTSDERVSIYKLTGAVMHYGNLKFKQKQREEQAEPDGTEVADKAAYLQGLNSADLLKALCYPRVKVGNEFVTKGQTVEQVYNAVGALAKAVYDKMFLWMVARINQQLDTKQPRQYFIGVLDIAGFEIFDFNSLEQLCINFTNEKLQQFFNHHMFVLEQEEYKKEGIEWEFIDFGMDLAACIELIEKPMGIFSILEEECMFPKATDMSFKNKLYEQHLGKSNNFQKPKPAKGKAEAHFSLIHYAGTVDYNITGWLDKNKDPLNETVVGLYQKSSVKTLALLFSGPASGEAEGGPKKGGKKKGSSFQTVSALFRENLNKLMTNLRSTHPHFVRCIIPNETKTPGAMEHELVLHQLRCNGVLEGIRICRKGFPSRILYADFKQRYKVLNASAIPEGQFIDSKKASEKLLASIDVDHTQYKFGHTKVFFKAGLLGLLEEMRDEKLAQLITRTQARCRGFLARVEYQKMVERRESIFCIQYNVRAFMNVKHWPWMKLYFKIKPLLKSAETEKEMANMKEEFEKTKEELAKSEAKRKELEEKMVTLTQEKNDLQLQVQSEADALADAEERCDQLIKTKIQLEAKIKEVTERAEDEEEINAELTAKKRKLEDECSELKKDIDDLELTLAKVEKEKHATENKVKNLTEEMAGLDETIAKLTKEKKALQEAHQQTLDDLQAEEDKVNTLTKAKTKLEQQVDDLEGSLEQEKKLRMDLERAKRKLEGDLKLAQESTMDIENDKQQLDEKLKKKEFEMSNLQSKIEDEQALAMQLQKKIKELQARIEELEEEIEAERASRAKAEKQRSDLSRELEEISERLEEAGGATSAQIEMNKKREAEFQKMRRDLEEATLQHEATAAALRKKHADSVAELGEQIDNLQRVKQKLEKEKSEMKMEIDDLASNMETVSKAKGNLEKMCRALEDQLSELKTKEDEQQRLINDLTTQRARLQTESGEFSRQLDEKDALVSQLSRGKQAFTQQIEELKRQLEEEIKAKSALAHALQSARHDCDLLREQYEEEQEGKAELQRAMSKANSEVAQWRTKYETDAIQRTEELEEAKKKLAQRLQDAEEHVEAVNAKCASLEKTKQRLQNEVEDLMIDVERTNAACAALDKKQRNFDKILSEWKQKYEETHAELEASQKESRSLSTELFKIKNAYEESLDQLETLKRENKNLQQEISDLTEQIAEGGKRIHELEKVKKQVEQEKSEIQAALEEAEASLEHEEGKILRIQLELNQVKSEIDRKIAEKDEEIDQLKRNHIRIVESMQSTLDAEIRSRNDAIRLKKKMEGDLNEMEIQLNHANRMAAEALKNYRSTQAILKDTQIHLDDALRGQEDLKEQLAMVERRANLLQAEIEELRATLEQTERSRKIAEQELLDASERVQLLHTQNTSLINTKKKLETDITQIQGEMEDIIQEARNAEEKAKKAITDAAMMAEELKKEQDTSAHLERMKKNLEQTVKDLQHRLDEAEQLALKGGKKQIQKLEARVRELEGEVESEQKRNVEAVKGLRKHERRVKELTYQTEEDRKNILRLQDLVDKLQAKVKSYKRQAEEAEEQSNVNLSKFRKLQHELEEAEERADIAESQVNKLRVKSREVHTKIISEE

SEQ ID NO: 91 - protein, 7100 aa, Bos taurus
MPNFQTRTRFYETTTTRTSDYEQSETSRPALAQPVPEKPVERKRVIRKKVDPSKFMTPYIAHSQKMQNLFSTNKYKENYEKAKGKPYAITTDTPELRRIKKVQDQLSEVKYRVDGDVAKTICHVDEKAKDIEHAKKVSQQVSKVLYKQNWEDTKDKYLLPPDAPELVQAIKNTAMFSKKLYTEDWEADKTMFYPYNDSPELRRVAQAQKALSDIAYKKGLAEQQTQFTSLPDPPDIEFAKKVTNQVSKQKYKEDYENKVKGKWSETPCFEIATARMNSNNISTKKYQEDFEHMKDQIYFMQTETPEYKVNKQAGVAASKVKYKQDYEKNKGKADYNVLPASENPLLRQLKAAGDALSDKLYKENYEKTKAKSINYCETPKFKLDTVLHNFSSDTKYKDSYLKDILGHYVGSYEDPYHTHCMRVSAQNSDKNYKAEYEEDRGKGFFPQTITQEYEAIKKLDQCKDHTYKVHPDKTKFTQVTDSPVLMQAQVNSKQLSDLNYKAKHENEKFKCHIPPDTPAFIQHKLNAYNLSDNIYKHDWEKTKAKKFDIKVDAIPLLAAKANTKIASDVMYKKDYEKSKGKMIGALSINDDPKMLHSLKTAKNQSDRLYKENYEKTKAKSMNYCETPKYQLDTLLKNFSEAKYKDSYVQNVLGHYIGSFEDPYQVHCLKISAQNSDKNYKAEYEEDKGKCYFPQTITQEYEAIKKLDQCKDHTYKVHPDKTKFTAVTDTPVLLQAQLNTKQLSDLNYKAKHEGEKFKCHIPADAPQFIQHRVNAYNLSDNIYKHDWEKTKAKKFDIKVDAIPLLAAKANTKIASDVMYKKDYEKSKGKMIGALSINDDPKMLHSLKTAKNQSDHEYRKDYEKSKTIYTAPLDMLPLTHAKKSQAIASDVDYKHLLHNYSYPPDSVNVDLAKKAYALQSDVEYKADYNSWMKGCGWMPFGSLEMEKAKRASDILNEKKYRQHPDTLKFTSIEDAPIIVQSKINQAQRSDVAYKAKGEEVIHKYSLPADLPQFIQAKVNAYNISENLYKADLKDLSKKGYDLRIDAIPIKAAKAARQAASDVQYKKDYEKAKGKMVGFQSLQDDPKLVHYMNVAKIQSDREYKKAYEKTKTRHNTPHDMVNIVAAKKAQDVASNVNYKHSLHHYTYLPDAMDLELSKNMMHIQSDNVYKEDYNNWMKGIGWIPIGSLEVEKVKKAGDALNEKKYRQHPDTLKFTSIVDSPVMVQAKQNTQQVSDILYKAKGEDVKHKYTMSPDLPQFLQAKCNAYNLSDVCYKRDWHDLIAKGTNVLGDAIPITAAKASRNIASDYKYKEAYEKAKGKQVGFRSLQDDPKLVHYMNVAKLQSDREYKKNYENTKTSYHTPGDMVSITAAKMAQDVATNVNYKQPIHHYTYLPDALSLEHIRNVNQIQSDNVYKDEYNHFFKGMGWIPIGSLEVEKVKKAGDALNEKKYRQHPDTIKFTSVPDSMGMVLAQHNTKQLSDLNYKVEGEKVKHKYTMDPDVPQFIQAKVNAYNMSDSHYKADWKKTLAKGYDLRPDAIPIVAAKSSRNIASDFKYKEAYEKTKGKQIGFRSLQDDPKLVHFMNVAKMQSDREYKKGYEASKTKYHTPLDMLSVTAAKKSQEVATNTNYKQPFHHYTLLPDALNVEHSRNAMQIQSDNLYKSDFTNWMKGIGWLPLESLEVEKAKKAGEILSEKKYRQHPEKLKFTYAMDTMEQALNKSNKLIMDKRLYTEKWNKDKTTIHVMPDTPDILLSRVNQITMSDKLYKAGWEEEKKKGYDLRPDAISIKAARASRDIASDYKYKQAYEQAKGKQIGFRSLEDDPKLVHFMQVAKMQSDREYKKAYEKSKTSFQTPVDMLSVVAAKKSQEVATNANYRNVIHTYNMLPDAMSLELAKNMMQIQSDNQYKADYADFMKGIGWLPLGSLEAEKNKKAMEIISEKKYRQHPDTLKYSTLMDSMNMVLAKNNAKIMNEHLYKQAWEADKTKVHIMPDIPQIILAKANAINISDKLYKLSLEEAKKKGYDLRTDAIPIKAAKASRDIASDYKYKHSYEKERGKMVGFRSLEDDPKLVHSMQVAKMQSDREYKKNYEKTKTSYHTPADMLSVTAAKDAQANITNTNYKHLIHKYILLPDAMNIQLSKNMNRIQSDNEYKQDYNEWYKGLGWSPAGSLEVEKAKKATEYASDQKYRQHPSNFQFTKLNDSMDMVLAKQNAHTMNKYLYTVDWNKDKTKIHVMPDTPDILQAKQNQTMYSQKLYKLGWEEALKKGYDLPVDAISVQAAKASRDIVSDFKYKQGYRKQIGHHIGFRSLQDDPKLVLSMNVAKMQSEREYKKDFEKWKTKFSSPVDMLGVVLAKKCQALVSDVDYKNYLHGWTCLPDQNDVIHAKKAYDLQSENLYKSDLEWLKGIGWSPLGSLEAEKNKRASEIISEKKYRQPPDRNKFTSIPDAMDIVLAKTNAKNRSDILYREAWDKDKTQIHIMPDTPDIILAKANLINTSDKFYRMGYEELRKKGYDLPVDAIPIKAAKASREIASEYKYKEGFRMQLGHHIGARNIKDDPKMMWSMHVAKIQSDREYKKDFEKWKTKFSSPVDMLGVVLAKKCQTLVSDIDYKNYLHQWTCLPDQSDVIHARRAYDLQSDNLYKSDLQWLRGIGWLPSGSLEDEKNKRASQILSDHVYRQHPDQFKFSSLMDSIPMVLAKNNAITMNHRLYTEAWDKDKTTVHIMPDTPEVLLAKQNQINYSEKLYKLGLDEAKRKGYDMRIDAIPIRAAKASRDIASEFKYKEGYRRQLGHHIGARAIHDDPKMMWSMHVAKIQSDREYKKDFEKWKTKFSSPVDMLGVVLAKKCQTLVSDIDYKNYLHQWTCLPDQSDVIHARQAYDLQSDNVYKSDLQWLRGIGWVPIGSLDVEKSKRASEILSDKLYRQPPDKFKFTSVTDSLEQVLAKNNAINMNKRLYTEAWDKDKTQVHIMPDTPEITLARTNKVNYSENLYKLAHEEAKKKGYDLRSDAIPIVAAKASRDIISDYKYKDGYRKQLGHHIGARDIKDDPKMMWSMHVAKIQSDREYKKDFEKWKTKFSSPVDMLGVVLAKKCQTLVSDIDYKNYLHQWTCLPDQSDVIHARQAYDLQSDNVYKSDLQWLRGIGWVPIGSVDVVKCKRAAEILSDNLYRQPPDTLKFTSVPDSLEQVLAKNNAINMNKRLYTEAWDKDKTQIHIMPDTPEIMLARQNKLNYSETLYKLANEEAKKKGYDLRSDAIPIVAAKASRDIISDYKYKDGYRKQLGHHIGARDIKDDPKMMWSMHVAKIQSDREYKKDFEKWKTKFSSPVDMLGVVLAKKCQTLVSDIDYKNYLHQWTCLPDQSDVIHARRAYDLQSDNIYKSDLQWLRGIGWVPIGSVDVVKCKRAAEILSDNLYRQRPDTLKFTSVPDSLEQVLAKNNAINMNKRLYTEAWDKDKTQIHMMPDTPEITLARQNKINYSENLYRQAMEEAKKEGYDLRSDAIPIVAAKASREIASDYKYKEAYRKQLGHHIGARAIHDDPKMMWSVHVAKMQSDREYKKDFEKYKTRFSSPVDMLGIVLAKKCQTLVSDVDYKHPLHEWTCLPDQNDIIHARKAYDLQSDNLYKSDLEWLKGIGWVPIGSVEVLKAKRAGEILSDNIYRQRPDTLKFTSVTDSPEQVLAKNNAINMNKRLYTEAWDNDKKTIHVMPDTPEIMLAKLNRINYSDKLYKLALEESRKEGYDLRLDAIPIQAAKASREIASDYKYKEGYRKQLGHHIGARNIKDDPKMMWSIHAGKLQSDLEYKKDFEKWKTKFSSPVDMMGLVQAKKCQILVSDIDYKNLLHEWTCLPDQNDIIQARKAYDLQSDAIYKADLEWLRGIGWVPIDSVGVEHAKRAGEILSERKYRQPAVQLKFTSITDTPEIVLAKNNALNVSKHLYTEAWDADKTSIHVMPDTPEILLAKSNSANISHKLYTKGWDESKMKDYDLRADAISIKSAKASRDIASDYKYKEAYEKHKGHHIGARSVEDDPRIMCAMNAGRIQSEREYKKEFQKWKTKFSSPVDMLGILLAKKCQTLVSDIDYRNYLHHWTCLPDQNDIIQARKAYDLQSDNVYKSDLEWLRGIGWMPEGSVEMNRVKNAQDLINERRYRTRPEALTFTSIVDTPEVVLAKANSLQMSEKLYQEAWNKDKTNISIPSDTPLILQAQINAMQISNKLYQKDWDEAKQKGYDLRADAIEIKHAKASREIASEYKYKEGYRKQLGHHVGFRTLQDDPKLVWSIHAAKIQSDREYRKAYEKSKGIHNTPLDMMSIVQAKKCQVLVSDIDYRNYLHQWICLPDQNDVIQAKKAYELQSDAIYKADLEWLRGIGWMPEGSPEVLRVKNAQHIFRDSVYRTPVVKLKYTSIVDTPEVVLAKSNAENISIPKYREVWDKDKTSIHIMPDTPEINLARTNALNVSNKLYREGWDEMKMSCDVRLDAIPIQAAKASREIASDYKYKLDHEKQKGHYVGTRTARDDNKIRWALIAGKIQNEREYRLHWAKWKSKFQSPPDMLSIEHSKNSQTLVSDIDYRHYLHQWTCMPDQNDVIQARKAYDLQSDAIYKADLEWLRGIGWMPADSVSVNHAKHMADIFNEKKYRTKIETLSFTPVDDRVDYVTAKHSGEILNDIKYRKDWNDTKSKYTLTETPQLHTAQEAARILDQYLYKESWEKQKATGYILPPDAVPFVHAHHSGDVQSELKYKAEHVKQKGHYVGVPTMRDDPKLVWFEHAGQIQNDRLYKENYHKTKAKIHIPPDMVSVLAAKEGQALASDIDYRNYLHQWICHPDQNDVIQARKAYDLQSDNIYKADLEWLRGIGWIPLDSVDHVRVTRNQEMMNQIKYKKDALANYPNFTSVVDPPEIVLAKINSVNQSDVKYKETFNKLIKGKYIFSPDTPYITHSKDMEKLYSTILYKRAWDGTKAYGYTLDERYIPIVGAKHADFVNSELKYKETYEKLKGHYLAGKEISEFPNVVHCLDFQKMRSLLNYRRHYEDTKANVHIPQDMMNHVLAKRCQYILSDLEYRHYFHQWTSLPEEPNVVRVRHAQEILSDNVYKDDLNWLKGIGCYVWDTPQILHAKKSYDLQSQILYTAAGKENLKNYNLVTDTPLYVTALQSGINASEVKYKENYHQTKDKYTTVLETVDYDRIKNLKDLFSSNLYKEAWDKVKATSYILPPNTVSLTHAKNQKYMASHIKYREEYEKFKALYTLPRSVEDDPNTARCLRVGKFNIDRLYRSVYEKNKMKIHIVPDMVEMVTAKDSQKKVSEIDYRLHLHEWICHPDLQVNSHVRKVTDQISDIVYKDDLTWLKGIGCYVWDTPEILHAKHAYDLRNDIKYKAHVLKTRNNYKLVTDTPVYVQAVKSGKQLSDAVYHYDYVHSIRGRVAPTTKTVDLDRALHAYKLQSENLYRKAGLHALPTGYRLPVDTPHFKHTKDTRYMSSYFKYKEVYEHMKAYGYTLGPNDVPFVNVRRVNNITSERLYRQLYHKLKDKIHTTPDTPEIRQVKKTQEAVSELIYKSDFFKMQGHMISLPYTPQVLHCRYVGDITSDIKYKEDLQVLRGMGCFLYDTPDMVRSRHLRKLWSHYLYTDKARKMRDKYKVVLDTPEYRKVQELKTHLSELVYRAAGRKQKSIFTSVPDTPDLTRAKRGQKLQSQYLYVELATKERPHHHAGNQTTALKHARHVKDMVSENKYKIQYEKMKDKYTPVPDTPILIRAKRAYWNASDLRYKETFQKTKGKYHTVKDALDIVYHRTVTDHISKIKYKENYMSQLGIWRSIPDRPEHFHHRAVTDAVSDVKYKQDLTWLKGIGCYAYDTPDFTLAEKNKTLYSKYKYKEVFERTKSNFKYVADCPINRHFKFATQLMNEKKYRADYEQRKDKYHLVVDEPRHLLAKIAGDQISQIKYRKNYEETKDKFTSIVDTPEHLRTTKVNKQISDILYKLEYNKAKPRGYTTIHDTPMLLHVRKVKDEVSDLKYKEVYQRTKSNCTIEPDAVHIKAAKDAYKVNTNLDYKKKYEATKAYWKWTPDRPDFIQAAKSTLQQSDFEYKLDREYLKGCKLSVTDDKDMVLALKNSIIESDLKYKEKHVKERGSCHAVPDTPQILLAKTVSSLVSENKYKSYVKKHLAQGSYTTLPETRDTIHVKEVTKNVSDTNYKKKFVKEKGKSNYSIMLEPPDVKHAMDVAKKQSNVAYKKDAKENLHYTTVADRPDIKKATQAAKQASEVEYRAKHRKEGSHGLSMLGRPDIEMAKKAAKLSSQVQYRENFNKEKGKTPKYNPKDSQLYKVMKDANTLASEVKYKADLKKLHKPVTDMKESLIMNHVLNTSHLASSYQYKKNYEKSKGHYHTIPDNLEQLHLKEATELQSIVKYKEKYEKERGKPMLDFETPTYITAKESQQMQSGKEYRKDYEESIKGRNLTGLEVTPALLHVKYATKIASEKEYRKDLEESIRGKGLSEMEDTPDMLRAKNATQILNEKEYKRDLELEVKGRGLNAMANETPDFLRARNATDIASQIKYKQSAEMEKANFTSVVDTPEIIHAQQVKNLSSQKKYKEDAEKCMSYYETVLDTPEMQRVRENQKNFSLLQYQYDLKNSKGKITVVQDTPEILRVKENQKNFSSVLYKEDVSPGTAIGKTPEMMRVKQTQDHISSVKYKEVIGQGTPIPDLPEVKRVKQTQKHISSVMYKENLGTGIPTPVTPEIERVKRNQENFSSNLSLKHEVTSKSIILTKYNSLKLPPHTPLPPVLYKENLGKGTPTPITPEMERVKRNQENFSSVLYKENMGKGTPLPVTPEMERIKHNQENISSVLYKENMRKATPTPVTPEMERAKRNQENISSVLYSDSFRKQIQGKAAYVLDTPEMRRVRETQRHISTVKYHEDFEKHKGCFTPVVTDPITERVKKNTQDFSDICYRGIQRKVVEMEQKRNDQDQETITGLRVWRTNPGSVFDYDPAEDNIQSRSLHMINAQAQRRSREQSRSASGLSISGGEEKSEHSEAAHLSTYSDGGVFFSAASTGYKHARTTELPQQRSSSVATQQTTVSSIPSHPSTAGKIFRAMYDYMAADADEVSFKDGDAIVNVQAIDEGWMYGTVQRTGRTGMLPANYVEAI

SEQ ID NO: 92 - protein, 1935 aa, Bos taurus
MSSDQEMAIFGEAAPYLRKSEKERIEAQNKPFDAKTSVFVVDPKESFVKATVQSREGGKVTAKTEAGATVTVKEDQVFPMNPPKYDKIEDMAMMTHLHEPAVLYNLKERYAAWMIYTYSGLFCVTVNPYKWLPVYNAEVVTAYRGKKRQEAPPHIFSISDNAYQFMLTGELDVSIGESGAGKTVNTKRVIQYFATIAVTGEKKKEEPTSGKMQGTLEDQIISANPLLEAFGNAKTVRNDNSSRFGKFIRIHFGTTGKLASADIETYLLEKSRVTFQLKAERSYHIFYQIMSNKKPELIEMLLITTNPYDYAYVSQGEITVPSIDDQEELIATDSAIDILGFTSDERVSIYKLTGAVMHYGNLKFKQKQREEQAEPDGTEVADKAAYLQSLNSADLLKALCYPRVKVGNEFVTKGQTVQQVYNAVGALAKAVYEKMFLWMVTRINQQLDTKQPRQYFIGVLDIAGFEIFDFNSLEQLCINFTNEKLQQFFNHHMFVLEQEEYKKEGIEWEFIDFGMDLAACIELIEKPMGIFSILEEECMFPKATDTSFKNKLYEQHLGKSNNFQKPKPAKGKAEAHFSLVHYAGTVDYNIAGWLDKNKDPLNETVVGLYQKSAMKTLAFLFAGGPSAEEGGGGKKGGKKKGSSFQTVSALFRENLNKLMTNLRSTHPHFVRCIIPNETKTPGAMEHELVLHQLRCNGVLEGIRICRKGFPSRILYADFKQRYKVLNASAIPEGQFIDSKKASEKLLGSIDIDHTQYKFGHTKVFFKAGLLGILEEMRDEKLAQLITRTQAICRGFLMRVEFRKMMERRESIFCIQYNVRAFMNVKHWPWMKLYFKIKPLLKSAETEKEMANMKEEFEKTKEELAKSEAKRKELEEKMVTLMQEKNDLQLQVQSEADGLADAEERCDQLIKTKIQLEAKIKELTERAEDEEEINAELTAKKRKLEDECSELKKDIDDLELTLAKVEKEKHATENKVKNLTEEMAGLDENIAKLTKEKKALQEAHQQTLDDLQAEEDKVNTLTKSKTKLEQQVEDLEGSLEQEKKLRMDLERAKRKLEGDLKLAQESTMDIENDKQQLDEKLKKKEFEMSNLQSKIEDEQALAMQLQKKIKELQARIEELEEEIEAERASRAKAEKLRSDLSRELEEISERLEEAGGATSAQIEMNKKREAEFQKMRRDLEEATLQHEATAAALRKKHADSVAELGEQIDNLQRVKQKLEKEKSEMKMEIDDLASNMETVSKAKGNLEKMCRTLEDQLSEVKSKEEEHQRLINELSVQKAHLHTESGEFSRQLEEKDALVSQLSRGKQAFTQQIEELKRQLEEETKAKSALAHALQSARHDCDLLREQYEEEQEGKAELQRALSKANSEVAQWRTKYETDAIQRTEELEEAKKKLAQRLQDAEEHVEVVNAKCASLEKTKQRLQNEVEDLMLDVERSNAACAALDKKQRNFDKVLSEWKQKYEETQADLEASQKESRSLSTELFKVKNAYEESLDQLETLKRENKNLQQEISDLTEQIAEGGKHIHELEKVKKQIEQEKGELQAALEEAEASLEHEEGKILRIQLELNQVKSEIDRKIAEKDEEIDQLKRNHLRVVESMQSTLDAEIRSRNDALRIKKKMEGDLNEMEIQLNHANRQATEAIKNLRNTQGVLKDTQLHLDDAIRGQDDLKEQLAMVERRANLMQAEIEELRASLEQTERSRRVAEQELLDASERVQLLHTQNTSLINTKKKLETDITQIQGEMEDIVQEARNAEEKAKKAITDAAMMAEELKKEQDTSAHLERMKKNMEQTVKDLQHRLDEAEQLALKGGKKQIQKLEARVRELENEVENEQKRNAEAVKGLRKHERRVKELTYQTEEDRKNVLRLQDLVDKLQSKVKAYKRQAEEAEEQSNVNLAKFRKLQHELEEAEERADIAESQVNKLRVKSREVHTKVISEE

SEQ ID NO: 93 - protein, 607 aa, Bos taurus
MKWVTFISLLLLFSSAYSRGVFRRDTHKSEIAHRFKDLGEEHFKGLVLIAFSQYLQQCPFDEHVKLVNELTEFAKTCVADESHAGCEKSLHTLFGDELCKVASLRETYGDMADCCEKQEPERNECFLSHKDDSPDLPKLKPDPNTLCDEFKADEKKFWGKYLYEIARRHPYFYAPELLYYANKYNGVFQECCQAEDKGACLLPKIETMREKVLASSARQRLRCASIQKFGERALKAWSVARLSQKFPKAEFVEVTKLVTDLTKVHKECCHGDLLECADDRADLAKYICDNQDTISSKLKECCDKPLLEKSHCIAEVEKDAIPENLPPLTADFAEDKDVCKNYQEAKDAFLGSFLYEYSRRHPEYAVSVLLRLAKEYEATLEECCAKDDPHACYSTVFDKLKHLVDEPQNLIKQNCDQFEKLGEYGFQNALIVRYTRKVPQVSTPTLVEVSRSLGKVGTRCCTKPESERMPCTEDYLSLILNRLCVLHEKTPVSEKVTKCCTESLVNRRPCFSALTPDETYVPKAFDEKLFTFHADICTLPDTEKQIKKQTALVELLKHKPKATEEQLKTVMENFVAFVDKCCAADDKEACFAVEGPKLVVSTQTALA

SEQ ID NO: 94 - protein, 381 aa, Ovis aries
MPFGNTHNKHKLNFKAEEEYPDLSKHNNHMAKALTLEIYKKLRDKETPSGFTLDDVIQTGVDNPGHPFIMTVGCVAGDEESYTVFKDLFDPIIQDRHGGFKPTDKHKTDLNHENLKGGDDLDPHYVLSSRVRTGRSIKGYALPPHCSRGERRAVEKLSVEALNSLTGEFKGKYYPLKSMTEQEQQQLIDDHFLFDKPVSPLLLASGMARDWPDARGIWHNDNKSFLVWVNEEDHLRVISMEKGGNMKEVFRRFCVGLQKIEEIFKKAGHPFMWNEHLGYVLTCPSNLGTGLRGGVHVKLAHLSKHPKFEEILTRLRLQKRGTGGVDTAAVGSVFDVSNADRLGSSEVEQVQLVVDGVKLMVEMEKKLEKGQSIDDMIPAQK

SEQ ID NO: 95 - protein, 897 aa, Ovis aries
MMMVLQPEGLGTGEGPFAGGGRGGGEYMEQEEDWDRDLLLDPAWEKQQRKTFTAWCNSHLRKAGTQIENIEEDFRNGLKLMLLLEVISGERLPRPDKGKMRFHKIANVNKALDFIASKGVKLVSIGAEEIVDGNLKMTLGMIWTIILRFAIQDISVEETSAKEGLLLWCQRKTAPYRNVNVQNFHTSWKDGLALCALIHRHRPDLIDYAKLRKDDPIGNLNTAFEVAEKYLDIPKMLDAEDIVNTPKPDEKAIMTYVSCFYHAFAGAEQAETAANRICKVLAVNQENEKLMEEYEKLASELLEWIRRTVPWLENRVGEPSMSAMQRKLEDFRDYRRLHKPPRVQEKCQLEINFNTLQTKLRLSHRPAFMPSEGKLVSDIANAWRGLEQAEKGYEDWLLSEIRRLQRLQHLAEKFQQKASLHEAWTRGKEEMLSQRDYETASLQEVRALLRRHEAFESDLAAHQDRVEHIAALAQELNELDYHEAASVNSRCQAICDQWDNLGTLTQKRRDALERMEKLLETIDQLQLEFARRAAPFNNWLDGAVEDLQDVWLVHSVEETQSLVTAHDQFKATLPEADRERGAILGIQGEIQKICQTYGLRPSSTNPYIALTPQDINTKWDTVRKLVPSRDQMLQEELTRQQMNERLRRQFAAQANAIGPWIQGKVEEGGRLAAGMAGSLEEQMAGLRQQEQNIINYKSNIDRLEGDHQLLQESLVFDNKHTVYSMEHIRVGWEQLLTSIARTINEVENQVLTRDAKGLSQEQLNEFRASFNHFDRKRNGMMEPDDFRACLISMGYDLGEVEFARIMTMVDPNAAGVVTFQAFIDFMTRETAETDTAEQVVASFKILAGDKNYITAEELRRELPAEQARRAPDRGAGAPAGALDYVAFSSALYGESDL

SEQ ID NO: 96 - protein, 842 aa, Ovis aries
MSRPLTDQEKRKQISVRGLAGVENVTELKKNFNRHLHFTLVKDRNVATPRDYYFALAYTVRDHLVGRWIRTQQHYYEKDPKRIYYLSLEFYIGRTLQNTMVNLALENACDEATYQLGLDMEELEEIEEDAGLGNGGLGRLAACFLDSMATLGLAAYGYGIRYEFGIFNQKISGGWQMEEADDWLRYGNPWEKARPEFTLPVHFYGRVEHTSQGAKWVDTQVVLAMPYDTPVPGYRNNVVNTMRLWSAKAPNDFNLKDFNVGGYIQAVLDRNLAENISRVLYPNDNFFEGKELRLKQEYFVVAATLQDIIRRFKSSKFGCLDPVRTNFDAFPDKVAIQLNDTHPSLAIPELMRILVDQERLEWEKAWEVTVKTCAYTNHTVLPEALERWPVHLIETLLPRHLQIIYEINQRFLNRVAAAFPGDVDRLRRMSLVEEGAVKRINMAHLCIAGSHAVNGVARIHSEILKKTIFKDFYELEPHKFQNKTNGITPRRWLVMCNPGLAEVIAERIGEEYIADLDQLRKLLSYVDDESFIRDVAKVKQENKLKFSAYLEKEYKVHINPNSLFDIQVKRIHEYKRQLLNCLHVITLYNRIKKEPNKFFVPRTVMIGGKAAPGYHMAKMIIRLITAIGDVVNHDPVVGDRLRVIFLENYRVSLAEKVIPAADLSEQISTAGTEASGTGNMKFMLNGALTIGTMDGANVEMAEEAGEENFFIFGMRVEDVERLDQKGYNAQEYYDRIPELRHIIDQLSSGFFSPKQPDLFKDIVNMLMHHDRFKVFADYEEYVKCQERVSALYKNPREWTRMVIRNIATSGKFSSDRTIAQYAREIWGVEPTRQRMPAPDEKI

SEQ ID NO: 97 - protein, 364 aa, Ovis aries; identical to SEQ ID NO: 82

SEQ ID NO: 98 - protein, 197 aa, Ovis aries
ILLNKMAPKKDVKKPAAAAAPAPAPAPAPAPAPAPPKEEKIDLSAIKIEFSKQQQDEFKEAFLLYDRTGDGKITLSQVGDVLRALGTNPTNAEVKKVMGNPSNEEMNAKKIEFEQFLPMLQAISNNKDQGTYEDFVEGLRVFDKEGNGTVMGAELRHVLATLGEKMKEEEVEALMAGQEDSNGCINYEAFVKHIMSN

SEQ ID NO: 99 - protein, 322 aa, Ovis aries
IGRLVTRAAFNTGKVDIVAINDPFIDLHYMVYMFQYDSTHGKFHGTVKAENGKLVINGKAITIFQERDPANIKWGDAGAEYVVESTGVFTTMEKAGAHLKGGAKKVIISAPSADAPMFVMGVNHEKYNNTLKIVSNASCTTNCLAPLAKVIHDHFGIVEGLMTTVHAITATQKTVDGPSGKLWRDGRGAAQNIIPASTGAAKAVGKVIPELNGKLTGMAFRVPTPNVSVVDLTCRLEKPAKYDEIKKVVKQASEGPLKGILGYTEDQVVSCDFNSDTHSSTFDAGAGIALNDHFVKLISWYDNEFGYSNRVVDLMVHMASKE

SEQ ID NO: 100 - protein, 294 aa, Ovis aries
MDAIKKKMQMLKLDKENALDRAEQAEADKKAAEDRSKQLEDELVSLQKKLKATEDELDKYSEALKDAQEKLELAEKKATDAEADVASLNRRIQLVEEELDRAQERLATALQKLEEAEKAADESERGMKVIESRAQKDEEKMEIQEIQLKEAKHIAEDADRKYEEVARKLVIIESDLERAEERAELSEGQVRQLEEQLRIMDQTLKALMAAEDKYSQKEDKYEEEIKVLSDKLKEAETRAEFAERSVTKLEKSIDDLEDELYAQKLKYKAISEELDHALNDMTSIESGSCQRRKP

SEQ ID NO: 101 - protein, 1141 aa, Ovis aries
MPEAKPAAKKAPAGKDAAAKPAPKEAPPQEAPAAPSTEAPPEDQSPTAEEPTGVFLKKPDSVSVENGKDTVIVAKLNGKELPAKPAVKWFKGKWLELGSKSGARFSFKESHDAASNVYTVELHITKVVLGDRGDYRIEVKAKDFCDSCTFNIDVESPRQDSAGQSLESFKRSSEAKSDTAGELDFSGLLKKRQVVEEEKKKKKDDDDLGIPPEIWELLKGAKKSEYERIAFQYGITDLRGMLKRLKKAKVEVKKSAAFTKKLDPAYQVDKGNKIKLVVEISDPDLPLKWFKNGQEIKPSSKYVFENVGKKRILTINKCTLADDAAYEVVVKDEKCFTELFVKVEPPVLIVTPLEDQQVFVGNRVEMAVEVSEEGAQVMWLKDGVELTREDSFKSRYRLKKDGKRHILIYAEVALEDKGHYKVMTNGDQCEAELIVEEKQLEVLQDIADLTVKASEQAVFKCEVSDEKVTGKWYKNGVEVRPSKRITISHTGRIHKLVIDDVRPEDEGDYTFVPDGYALSLSAKLNFLGAGLSPLPPEPPKIHLDCSGQTSENSVVVVAGNKLRLDVSITGEPRPVATWMKEDEVFTGTEGRVRIEQRGDISSFVIESAERGDEGRYTIKVTNPAGEDVASILLKVVDVPDPPEAVRVTSVGEDWAVLVWEPPKYDGGRPVTGYLLERKKKGSQRWMKLNFEVFTETTYESTNMIEGILYEMRVFAVNAIGVSQPSMNTKPFMPIAPTSEPQHLTVEDVTDTTTTLKWRPPEKIGAGGIDGYLIEYCVEGSDEWIPANTEPVERCGFTVKNLPTGAKITFRVVGVNIAGRSQPATLAQPVTIREIVEQPKIRLPRHLRQTYVRKVGEHLNLVIPFQGKPRPQVVWTKGGAPIDTNHVHVRTSDFDTVFFVRQAARSDSGEYELTVQIENMKDTATIRIRVVEKAGPPENVMVKEVWGTNALVEWQPPRDDGNSEITGYLVQKADKKTMEWFTVYERNRHTSCTVSDLIMGNEYYFRVYSENICGLSDLPGVSKNTARIVKTGMTLKLPEYKEHDFRTPPKFLTPLFDRVVVAGYAAALNCAVRGHPKPKVVWMKNKMEIREDPKFLMTNHQGVLTLNIRRPSPFDSGTYSCRAVNELGEALAECKLEVRGEP

SEQ ID NO: 102 - protein, 1483 aa, Ovis aries
MSLVAVPFYQKRHKHFDQSYRNIQTRYLLDEYAAKKKRASAQSSLQRSVTRKSSSQRESSRAALGETTLGETTLGRTTCPLCAKRMSSALEEEAQERKRRYQSMVAAYGEAKRERYLSELAQLEEDVHVARTHAREELDRLALQHAGRHGLHHAVDDRLAWERHWFEDRISRAPEILVRLRSHTVWERMSVKLCFTVQGFPTPVVQWYKDGSLICQAGEPGKYRIDSKYGVHTLEINRADFDDTATYSAVATNVHGQVSTNAAVVVRRFRGDEEPFHSVGLPIGLPLSSVIPYTHFDVQFIEKFGVTFRREGETLTLKCTLLVTPDLKRVQPRAEWYRDDVLLKESKWTKMFFGEGQASLSFSHLNKDDEGLYTLRIVSRGGVTDHSAFLFVRDADPLVTGAPGAPMDVHCHDANRDYVIVTWKPPNTTKESPVIGYFIDRCEVGTDNWVQCNDAPVKICKYPVTGLFEGRSYIFRVRAVNSAGISRPSRVSEAVAALDPVDLRRLQAVHLEGEKDIVIYQEDLEGEVQIPGPPTNVHASETSRTYVVLSWDPPEPRGKEPLMYFIEKSMVGSGSWQRVNAQTAVRSPRYAVFDLAEGKPYVFRVLSANKHGLSEPSEITAPIQAQDTIVVPSAPGRVLASRNTRTSVVVQWDRPKHEEDLLGYYVDCSVAGSNVWEPCNHKPIGYNRFVVHGLTTGEQYIFRVKAVNAVGTSENSQESDVIKVQAALTVPSHPYGITLLNCDGHSMILGWKVPKFSGGSPILGYYIDKREAHHKNWHEVNSSPLKERILKVEGLTEGSLYEFKIAAANMAGMGSQQTELFKCEAWTMPEPGPAYDLTFCEVRDTSLVLLWKPPVYSGSSPVSGYFVDYKEEDSGEWLTVNEVTTPHRYLKVCDLHQGKTYVFRVRAVNASGVGRPSDTSEPVLVEARPGTKEVSAGVDEEGNIYLSFDCPEVTDASHFTWCKSYEDIADDDRFKVETAGDHSKLYFKNLDKEDLGTYSVSVSDTDGVSSSFVLDEEELERLKALSNEIKNPTIPLKSELAYEIFDKGQVRFWLQAEHLSPDSSYRFIINDREVADSETHRIKCDKSTGVIEMVMDRFTVDNEGTYTVQIQDGKAKSQSSLVLIGDAFKAVLEEAEFQRKEFLRKQGPHFAEYLHWDVTEECEVRLVCKVANTKKETVFKWLKDDVLYETEKMPDLEKGVCELLIPKLSKKDHGEYKATLKDDRGQDVSTLEIAGKVYEDMILAMSRVCGASASPLKILCTPEGIRLQCFMKYFTEEMKVNWYHKDAKIASSEHMRIGGSEEMAWLQICEPTEKDKGKYTFEILDGKKNHQRSLDLSGQAFDEAFAEFQQLKAAAFAEKNRGKVIGGLPDVVTIMEGKTLNLTCTVFGNPDPEVVWFKNDKDIELSDHFSVKVEQAKYVSMTIKGVTSEDSGKYSMHVKNKYGGEKIDVTVSVYKHGEEIPDVVLPQQAKPKLLPAAAPSPAH

SEQ ID NO: 103 - protein, 434 aa, Ovis aries
MAMQKIFAREILDSRGNPTVEVDLHTAKGRFRAAVPSGASTGIYEALELRDGDKSRYLGKGVLKAVEHINKTLGPALLEKKLSVVDQEKVDKFMIELDGTENKSKFGANAILGVSLAVCKAGAAEKGVPLYRHIADLAGNPELILPVPAFNVINGGSHAGNKLAMQEFMILPVGASSFREAMRIGAEVYHHLKGVIKAKYGKDATNVGDEGGFAPNILENNEALELLKTAIQAAGYPDKVVIGMDVAASEFYRNGKYDLDFKSPDDPARHISGEKLGELYKSFIKNYPVVSIEDPFDQDDWATWTSFLSGVNIQIVGDDLTVTNPKRIAQAVEKKACNCLLLKVNQIGSVTESIQACKLAQSNGWGVMVSHRSGETEDTFIADLVVGLCTGQIKTGAPCRSERLAKYNQLMRIEEALGDKAVFAGRKFRNPKAK

SEQ ID NO: 104 - protein, 170 aa, Ovis aries
QAPKKAKRRAAAEGGSSNVFSMFDQTQIQEFKEAFTVIDQNRDGIIDKEDLRDTFAAMGRLNVKNEELDAMMKEASGPINFTVFLTMFGEKLKGADPEDVITGAFKVLDPEGKGTIKKQFLEELLTTQCDRFSQEEIRNMWAAFPPDVGGNVDYKNICYVITHGDAKDQE

SEQ ID NO: 105 - protein, 1937 aa, Ovis aries
MSSDQEMAIFGEAAPYLRKSEKERIEAQNKPFDAKTSVFVADPKESFVKATVQSREGGKVTAKTEAGATVTVKEDQVFPMNPPKFDKIEDMAMMTHLHEPAVLYNLKERYAAWMIYTYSGLFCVTVNPYKWLPVYNAEVVAAYRGKKRQEAPPHIFSISDNAYQFMLTDRENQSILITGESGAGKTVNTKRVIQYFATIAVTGEKKKEEPTSGKMQGTLEDQIISANPLLEAFGNAKTVRNDNSSRFGKFIRIHFGTTGKLASADIETYLLEKSRVTFQLKAERSYHIFYQIMSNKKPELIEMLLITTNPYDYAFVSQGEITVPSIDDQEELMATDSAIEILGFTSDERVSIYKLTGAVMHYGNLKFKQKQREEQAEPDGTEVADKAAYLQGLNSADLLKALCYPRVKVGNEFVTKGQTVEQVYNAVGALAKAIYDKMFLWMVTRINQQLDTKQPRQYFIGVLDIAGFEIFDFNSLEQLCINFTNEKLQQFFNHHMFVLEQEEYKKEGIEWEFIDFGMDLAACIELIEKPMGIFSILEEECMFPKATDTSFKNKLYEQHLGKSNNFQKPKPAKGKAEAHFSLIHYAGTVDYNITGWLDKNKDPLNETVVGLYQKSSVKTLAFLFSGAASAEAEGGGAKKGGKKKGSSFQTVSALFRENLNKLMTNLRSTHPHFVRCIIPNETKTPGAMEHELVLHQLRCNGVLEGIRICRKGFPSRILYADFKQRYKVLNASAIPEGQFIDSKKASEKLLGSIDVDHTQYKFGHTKVFFKAGLLGLLEEMRDEKLAQLITRTQARCRGFLARVEYQRMVERRESIFCIQYNVRAFMNVKHWPWMKLYFKIKPLLKSAETEKEMANMKEEFEKTKEELAKSEAKRKELEEKMVTLTQEKNDLQLQVQSEADALADAEERCDQLIKTKIQLEAKIKEVTERAEDEEEINAELTAKKRKLEDECSELKKDIDDLELTLAKVEKEKHATENKVKNLTEEMAGLDETIAKLTKEKKALQEAHQQTLDDLQAEEDKVNTLTKAKTKLEQQVDDLEGSLEQEKKLRMDLERAKRKLEGDLKLAQESTMDIENDKQQLDEKLKKKEFEMSNLQSKIEDEQALGMQLQKKIKELQARIEELEEEIEAERASRAKAEKQRSDLSRELEEISERLEEAGGATSAQIEMNKKREAEFQKMRRDLEEATLQHEATAAALRKKQGGVGEQLDNLQRVKQKLEKEKSEMKMEIDDLASNMETVSKAKGNLEKMCRTLEDQLSELKTKEDEQQRLINDLTTQRARLQTESGEFSRQLDEKDALVSQLSRGKQAFTQQIEELKRQLEEEIKAKSALAHALQSARHDCDLLREQYEEEQEAKAELQRALSKANSEVAQWRTKYETDAIQRTEELEEAKKKLAQRLQDAEEHVEAVNAKCASLEKTKQRLQNEVEDLMIDVERTNAACAALDKKQRNFDKILSEWKQKYEETHAELEASQKESRSLSTELFKIKNAYEESLDQLETLKRENKNLQPTEEISDLTEQIAEGGKRIHELEKVKKQVEQEKSEIQAALEEAEASLEHEEGKILRIQLELNQVKSEIDRKIAEKDEEIDQLKRNHIRVVESMQSTLDAEIRSRNDAIRLKKKMEGDLNEMEIQLNHANRMAAEALKNYRNTQAILKDTQLHLDDALRGQEDLKEQLAMVERRANLLQAEIEELRATLEQTERSRKIAEQELLDASERVQLLHTQNTSLINTKKKLETDITQIQGEMEDIIQEARNAEEKAKKAITDAAMMAEELKKEQDTSAHLERMKKNLEQTVKDLQHRLDEAEQLALKGGKKQIQKLEARVRELEAEVESEQKRNVETVKGLRKHERRVKELTYQTEEDRKNILRLQDLVDKLQAKVKSYKRQAEEAEEQSNVNLSKFRKLQHELEEAEERADIAESQVNKLRVKSREVHTKIISEE

SEQ ID NO: 106 - protein, 6911 aa, Ovis aries
MADDEEYEEVVEYYTEETVYEEVPGETITEFYETTTTRTSDYEQSDTSRPALAKPVPEKPVERKRVIRKKVDPSKFMTPYIAHSQKMQDLFSTNKYKENYEKAKGQPYAITTDTPELRRIKKVQDQLSEVKYRTDGDVAKTICHVDEKAKDIEHAKKVSQQVSKVLYKQNWEDTKDKYLLPPDAPELVQAIKNTAMFSKKLYTEDWEADKTLFYPYNDSPELRRVAQAQKALSDITYKKGLAEQQTQFTSLPDPPDIEFAKKVTNQVSKQKYKEDYENKVKGKWSETPCFEIATARMNSQNISTKKYQEDFENTKDQIYFMQTETPEYKVNKQAGVAASKVKYKQDYEKNKGKADYNVLPASENPLLRQLKAAGDALSDKLYKENYEKTKAKSINYCETPKFKLDTVLHNFSSDTKYKDSYLKNILGHYVGSYEDPYHTHCMRVSAQNSDKNYKAEYEEDRGKGFFPQTITQEYEAIKKLDQCKDHTYKVHPDKTKFTQVTDSPVLVQAQVNSKQLSDLNYKAKHENEKFKCHIPPDTPAFIQHKVNAYNLSDNIYKHDWEKTKAKKFDIKVDAIPLLAAKANTKIASDVMYKKDYEKSKGKMIGALSINDDPKMLHSLKTAKNQSDRLYKENYEKTKAKSMNYCETPKYQLDTLLKNFSEAKYKDSYVQNVLGHYIGSFEDPYREHCMKVAAQNSDKNYKAEYEEDKGKCYFPQTITQEYEAIKKLDQCKDHTYKVHPDKTKFTAVTDTPVLLQAQLNTKQLSDLNYKAKHEGEKFKCHIPADAPQFIQHRVNAYNLSDNIYKHDWEKTKAKKFDIKVDAIPLLAAKANTKIASDVMYKKDYEKSKGKMIGALSINDDPKMLHSLKTAKNQSDREYRKDYEKSKTIYTAPLDMLPLTHAKKSQEIASDVDYKHLLHNYSYPPDSVNVDLAKKAYALQSDVEYKADYNSWMKGCGWVPFGSLEMEKAKRASDILNEKKYRQHPDTLKFTSIEDAPIIVQSKINQAQRSDVAYKAKGEEVIHKYSLPADLPQFIQAKVNAYNISENMYKADLKDLSKKGYDLRIDAIPIKAAKAARQAASDVQYKKDYEKAKGKMVGFQSLQDDPKLVHYMNVAKIQSDREYKKDYEKTKTRHNTPHDMVNIVAAKKAQDVASNVNYKHSLHHYTYLPDAMDLELSKNMMHIQSDNVYKEDYNNWMKGIGWIPIGSLEVEKVKKAGDALNEKKYRQHPDTLKFTSIVDSPVMVQAKQNTQQVSDILYKAKGEDVKHKYTMSPDLPQFLQAKCNAYNLSDVCYKRDWHDLIAKGTNVLGDAIPITAAKASRNIASDYKYKEAYEKAKGKQVGFRSLQDDPKLVHYMNVAKLQSDREYKKNYENTKTSYHTPGDMVSITAAKMAQDVATNVNYKQPIHHYTYLPDALSLEHIRNVNQIQSDKVYKDEYNHFFKGIGWIPIGSLEVEKVKKAGDALNEKKYRQHPDTIKFTSVPDSMGMVLAQHNTKQLSDLNYKVEGEKVKHKYTLDPDVPQFIQAKVNAFNMSDSHYKADWKKTLAKGYDLRPDAIPIVAAKSSRNIASDFKYKEGYEKAKGKQIGFRSLQDDPKLVHFMNVAKMQSDREYKKGYEASKTKYHTPLDMLSVTAAKKSQEVATNTNYKQPFHHYTLLPDALNVEHSRNAMQIQSDNLYKSDFTSWMKGIGWMPLESLEVEKAKKAGEILSEKKYRQHPEKLKFTYAMDTMEQALNKSNKLIMDKRRYTEKWNKDKTTIHVMPDTPDILLSRVNQITMSDKLYKAGWEEEKKKGYDLRPDAIPIKAARASRDIASDYKYKQAYEQAKGKQIGFRSLEDDPKLVHFMQVAKMQSDREYKKAYEKSKTSFHTPVDMLSVVAAKKSQEVATNANYRNVIHNYNMLPDAMSLELAKNMMQIQSDNQYKADYADFMKGIGWLPLGSLEAEKNKKAMEIISEKKYRQHPDTLKYSTLMDSMNMVLAKNNAKIMNEHLYKQAWEADKTKVHIMPDIPQIILAKANAINMSDKLYKLSLEEAKKKGYDLRTDAIPIKAAKASRDIASDYKYKHNYEKERGKMVGFRSLEDDPKLVHSMQVAKMQSDREYKKNYEKTKTSYHTPADMLSVTAAKDAQANITNTNYKHLIHKYILLPDAMNIQLSKNMNRLQSDNEYKQDYNEWYKGLGWSPAGSLEVEKAKRATEYASDQKYRQHPSNFQFSKLNDSMDMVLAKQNAHTMNKYLYTVDWNKDKTKIHVMPDTPDILQAKQNQTIYSQKLYKLGWEEALKKGYDLPVDAISVQAAKASRDIVSDFKYKQGYRKQIGHHIGFRSLQDDPKLVLSMNVAKMQSEREYKKDFEKWKTKFSSPVDMLGVVLAKKCQALVSDVDYKNYLHGWTCLPDQNDVIHAKKAYDLQSENLYKSDLEWLKGIGWSPLGSLEAEKNKRASEIISEKKYRQPPDRNKFTSIPDAMDIVLAKTNAKNRSDILYREAWDKDKTQVHIMPDTPDIILAKANLINASDKFYRMGYEELRKKGYDLPVDAIPIKAAKASREIASEYKYKEGFRKQLGHHIGARNIKDDPKMMWSMHVAKIQSDREYKKDFEKWKTKFSSPVDMLGVVLAKKCQTLVSDIDYKNYLHQWTCLPDQSDVIHARRAYDLQSDNLYKSDLQWLKGIGWLPSGSLEDEKNKRATQILSDHVYRQHPDKFKFSSLMDSIPMVLAKNNAITMNHRLYTEAWDKDKTTVHIMPDTPGVLLAKQNQINYSEKLYKLGLDEAKRKGYDMRIDAIPIRAAKASRDIASEFKYKEGYRKQLGHHIGARAIHDDPKMMWSMHVAKIQSDREYKKDFEKWKTKFSSPVDMLGVVLAKKCQTLVSDIDYKNYLHQWTCLPDQSDVIHARQAYDLQSDNVYKSDLQWLKGIGWVPIGSLDVEKCKRASEILSDKLYRQPPDKFKFTSVTDSLEQVLAKNNAINMNKRLYTEAWDKDKTQVHIMPDTPEITLARMNKVNYSESLYKLAHEEAKKKGYDLRSDAIPIVAAKASRDIISDYKYKDGYRKQLGHHIGARDIQDDPKMMWSMHVAKIQSDREYKKDFEKWKTKFSSPVDMLGVVLAKKCQTLVSDIDYKNYLHQWTCLPDQSDVIHARRAYDLQSDNVYKSDLQWLRGIGWVPIGSVDVVKCKRAAEILSDNLYRQPPDTLKFTSVPDSLEQVLAKNNAINMNKRLYTEAWDKDKTQVHIMPDTPEIMLARQNKLNYSETLYKLANEEAKKKGYDLRSDAIPIVAAKASRDIISDYKYKDGYRKQLGHHIGARDIKDDPKMMWSMHVAKIQSDREYKKDFEKWKTKFSSPVDMLGVVLAKKCQTLVSDIDYKNYLHQWTCLPDQSDVIHARQAYDLQSDNIYKSDLQWLRGIGWVPIGSVDVVKCKRAAEILSDNLYRQRPDTLKFTSVPDSLEQVLAKNNAINMNKRLYTEAWDKDKTQIHMMPDTPEITLARQNKINYSENLYRQAMEEAKKEGYDLRSDAIPIVAAKASREIASDYKYKEAYRKQLGHHIGARAIHDDPKMMWSVHVAKMQSDREYKKDFEKYKTRFSSPVDMLGIVLAKKCQTLVSDVDYKHPLHEWTCLPDQNDIIHARKAYDLQSDNLYKSDLEWLKGIGWVPIGSFDVVKAKRAGEILSDNIYRQRPDTLKFTSVTDSPEQVLAKNNAINMSKRLYTEAWDNDKKTIHVMPDTPEIMLAKLNRINYSDKLYKLALEESKKEGYDLRLDAIPIQAAKASREIASDYKYKEGYRKQLGHHIGARNIKDDPKMMWSIHATKLQSDREYKKDFEKWKTKFSSPVDMMGLVQAKKCQILVSDIDYKNLLHEWICLPDQNDVIQARKAYDLQSDAIYKADLEWLRGIGWVPIGSVGVENVKRAGEILSERKYRQPAVQLKFTSITDTPEIVLAKNNALNMSKHLYTEAWDADKTSIHVMPDTPEILLAKSNSANISHKLYTKGWDESKMKDYDLRADAISIKSAKASRDIASDYKYKEAYEKQKGHHIGARSVEDDPKIMCAMNAGKIQSEREYKKEFQKWKTKFSSPVDMLGILQAKKCQILVSDIDYRNYLHHWTCLPDQNDIIQARKAYDLQSDAIYKADLEWLRGIGWMPEGSPEVLRVKNAQHIFRDSVYRTPVVKLKYTSIVDTPEVVLAKSNAENISIPKYREVWDKDKTSIHIMPDTPEINLARTNALNVSNKLYREGWDEMKMSCDVRLDAIPIQAAKASREIASDYKYKLDHEKQKGHYVGTRTARDDNKIRWALIAGKIQNEREYRMHWAKWKSKFQSPPDMLSIEHSKNSQTLVSDIDYRHYLHQWTCMPDQNDVIQARKAYDLQSDAIYKADLEWLRGIGWMPADSVSVNHAKHMGDIFNEKKYRTKIETLNFTPVDDRVDYVTAKHSGEILNDIKYRKDWNDTKSKYTLTETPQLHAAQEAARILDQYLYKESWEKQKATGYILPPDAVPFVHAHHSGDVQSELKYKAEHVKQKGHYVGVPTMRDDPKLVWFEHAGQIQNDRLYKESYHKTKAKIHIPPDMVSVLAAKEGQALASDIDYRTYLHQWICHPDQNDVIQARKAYDLQSDNIYKADLEWLRGIGWMPLDSVDHVRVTRNQEMVNQIKYKKDALANYPNFTSVVDPPEIVLAKINAVNQSDVKYKETFNKLIKGKYIFSPDTPYITHSKDMEKLYSTILYKRAWEGTKAYGYTLDERYIPIVGAKHADFVNSELKYKETYEKLKGHYLAGKEISEFPNVVHCLDFQKMRSLLNYRRHYEDTKANVHIPQDMMNHVLAKRCQYILSDLEYRHYFHQWTSLPEEPNVVRVRHAQEILSDNVYKDDLNWLKGIGCYVWDTPQILHAKKSYDLQSQILYTAAGKENLKNYNLVTDTPLYVTALQSGINASEVKYKENYHQIKDKYTTVLETVDYDRTRNLKDLFSSNLYKEAWDKVKATSYILPPNTVSLTHAKNQKHMASYVKYREEYDKFKALYTLPRSVEDDPNTARCLRVGKFNIDRLYRSVYEKNKMNIHIVPDMVEMVTAKDSQKKVSEIDYRLHLHEWICHPDLQVNSHVRKVTDQISDIVYKDDLTWLKGIGCYVWDTPEILHAKHAYDLRNDIKYKAHVLKTRNNYKLVTDTPVYVQAVKSGKQLSDAVYHYDYVHSIRGRVAPTTKTVDLDRALHAYKLQSENLYRKAGLHALPTGYRLPVDTPHFKHTKDTRYMSSYFKYKEVYEHMKAYGYTLGPNDVPFVNVRRVNNITSERLYRQLYHKLKDKIHTTPDTPEIRQVKKTQEAVSELIYKSDFFKMQGHMISMPYTPHVLHCRYVGDITSDIKYKEDLQILRGLGCFLYDTPDMVRSRHLRKLWSHYLYTDKARKMRDKYQVVLDTPEYRTVQELKTHLSELVYRASGRKQKSIFTSVPDTPDLIRAKRGQKLQSQYLYVELATKERPHHHAGNQTTALKHARHVKDMVSENKYKIQYEKMKDKYTPVPDTPILIRAKRAYWNASDLRYKETFQKTKGRYHTVKDALDIVYHRTVTDHISKIKYKENYMSQLGIWRSIPDRPEHFHHRAVTDAVSDVKYKADLTWLKGIGCYAYDTPDFTLAEKNKTLYSKYKYKEVFERTKSNFKYVADCPINRHFKFATQLMNEKKYRADYEQRKDKYHLVVDEPRHLLAKIAGDQISQIRYRKNYEKTKDKFTSIVDTPEHLRTTKVNKQISDILYKLEYNKAKPRGYTTIHDTPMLLHVRKVKDEVSDLKYKEVYQRTKSNCTIEPDAVHIKAAKDAYKVNTNLDYKKQYEATKAYWKWTPDRPDFIQAAKSTLQQSDFEYKLDREYLKGCKLSVTDDKDMVLALKNSIIESDLKYKEKHVKERGSCHAVPDTPQILLAKTVSSLVSENKYKQYVKKHLAQGSYTTLPETRDTIHVKEVTKNVSDTNYKKKFVKEKGKSNYSIMLEPPDVKHAMDVAKKQSNMKVAYKKDAKENLHYTTVADRPDIKKATQAAKQASEVEYRAKHRKEGSHGLSMLGRPDIEMAKKAAKLSSQVKYRENFNKEKGKTPKYNPKDSQLYKVMKDANTLASEVKYKADLKKLHKPVTDMKESLIMNHVLNTSHLASSYQYKKNYEKSKGHYHTIPDNLEQLHLKEATELQSIVKYKEKYEKERGKPMLDFETPTYITAKESQQMQSGKEYRRDYEESIKGRNLTGLEVTPALLHVKYATKIASEKEYRKDLEESIRGKGLSEMEDTPDMLRAKNATQILNEKEYKRDLELEVKGRGLNAMANETPDFLRARNATDIASQIKYKQSAEMEKANFTSVVDTPEIIHAQQVKNLSSQKKYKEDAEKCMSYYETVLDTPEMQRVRENQKNFSLLQYQYDLKNSKGKITVVQDTPEILRVKENQKNFSSVLYKEDVSPGTAIGKTPEMMRVKQTQDHISSVKYKEVIGQGTPIPDLPEVKRVKQTQKHISSVMYKENLGTGIPTPVTPEIERVKRNQENFSSVLYKENLGKGTPTPITPEMERVKRNQENFSSILYKENLSKGTPLPVTPEMERVKRNQENFSSVLYKENVGKGTPTPVTPEMQRVKRNQENISSVLYKENLGKATPTPFTPEMERVKRNQENFSSVLYKENMRKATPTPVTPEMERAKRNQENISSVLYSDSFRKQIQGKAAYVLDTPEMRRVRETQRHISTVKYHEDFEKHKGCFTPVVTDPITERVKKNMQDFSDISYRGIQRKVVEMEQKRNDQDQETVTGLRVWRTNPGSVFDYDPAEDNIQSRSLHMINAQAQRRSREQSRSASGLSVSGGEEKSEHSEAAHLSTYSDGGVFFSATSTGYKHARTTELPQQRSSSVATQQTTVSSIPSHPSTAGKIFRAMYDYMAADADEVSFKDGDAIVNVQAIDEGWMYGTVQRTGRTGMLPANYVEAI

SEQ ID NO: 107 - protein, 1948 aa, Ovis aries
MSSDQEMAIFGEAAPYLRKSEKERIEAQNKPFDAKTSVFVADPKESFVKATVQSREGGKVTAKTEAGATVTVKEDQVFPMNPPKFDKIEDMAMMTHLHEPGVLYNLKERYAAWMIYTYSGLFCVTVNPYKWLPVYNAEVVTAYRGKKRQEAPPHIFSISDNAYQFMLTDRENQSILITGESGAGKTVNTKRVIQYFATIAVTGEKKKEEPTSGKMQGTLEDQIISANPLLEAFGNAKTVRNDNSSRFGKFIRIHFGTTGKLASADIETYLLEKSRVTFQLKAERSYHIFYQIMSNKKPELIEMLLITTNPYDYAFVSQGEITVPSIDDQEELMATDSAIEILGFTSDERVSIYKLTGAVMHYGNLKFKQKQREEQAEPDGTEVADKAAYLQSLNSADLLKALCYPRVKVGNEFVTKGQTVQQVYNAVGALAKAVYEKMFLWMVTRINQQLDTKQPRQYFIGVLDIAGFEIFDFNSLEQLCINFTNEKLQQFFNHHMFVLEQEEYKKEGIEWEFIDFGMDLAACIELIEKPMGIFSILEEECMFPKATDTSFKNKLYEQHLGKSNNFQKPKPAKGKAEAHFSLVHYAGTVDYNIAGWLDKNKDPLNETVVGLYQKSAMKTLAFLFAGGPSAEVHVSEGGGGKKGGKKKGSSFQTVSALFRENLNKLMTNLRSTHPHFVRCIIPNETKTPGAMEHELVLHQLRCNGVLEGIRICRKGFPSRILYADFKQRYKVLNASAIPEGQFIDSKKASEKLLGSIDIDHTQYKFGHTKVFFKAGLLGILEEMRDEKLAQLITRTQAICRGFLTRVEFRKMMERSRESIFCIQYNVRAFMNVKHWPWMKLYFKIKPLLKSAETEKEMANMKEEFEKTKEELAKSEAKRKELEEKMVTLMQEKNDLQLQVQSEADGLADAEERCDQLIKTKIQLEAKIKELTERAEDEEEINAELTAKKRKLEDECSELKKDIDDLELTLAKVEKEKHATENKVKNLTEEMAGLDENIAKLTKEKKALQEAHQQTLDDLQAEEDKVNTLTKSKTKLEQQVDDLEGSLEQEKKLRMDLERAKRKLEGDLKLAQESTMDIENDKQQLDEKLKKKEFEMSNLQSKIEDEQALGMQLQKKIKELQARIEELEEEIEAERASRAKAEKQRSDLSRELEEISERLEEAGGATSAQIEMNKKREAEFQKMRRDLEEATLQHEATAAALRKKHADSVAELSEQIDNLQRVKQKLEEEGSNLTVQALDIISNFKIINRYYYIEGNLEKMCRTLEDQLSEVKSKEEEHQRLINELSAQKARLHTESGEFSRQLDEKDALVSQLSRGKQAFTQQIEELKRQLEEETKAKSALAHALQSARHDCDLLREQYEEEQEAKAELQRSMSKANSEVAQWRTKYETDAIQRTEELEEAKKKLAQRLQEAEEHVEAVNAKCASLEKTKQRLQNEVEDLMLDVERSNAACAALDKKQRNFDKVLSEWKQKYEETQAELEASQKESRSLSTELFKVKNAYEESLDQLETLKRENKNLQQEISDLTEQIAEGGKHIHELEKVKKQIEQEKGELQAALEEAEASLEHEEGKILRIQLELNQVKSEIDRKIAEKDEEIDQLKRNHLRVVESMQSTLDAEIRSRNDALRIKKKMEGDLNEMEIQLNHANRQATEATKNLRNTQGVLKDTQLHLDDAIRGQDDLKEQLAMVERRANLMQAEIEELRASLEQTERSRRVAEQELLDASERVQLLHTQNTSLINTKKKLETDITRVHGSVVGSGIISKHLTFVYISVGCAFQAAMMAEELKKEQDTSAHLERMKKNLEQTVKDLQHRLDEAEQLALKGGKKQIQKLEARVRELENEVENEQKRNAEAVKGLRKHERRVKELTYQTEEDRKNVLRLQDLVDKLQSKVKAYKRQAEEAEEQANVNLSKFRKLQHELEEAEERADIAESQVNKLRVKSREVHTKVISEE

SEQ ID NO: 108 - protein, 607 aa, Ovis aries
MKWVTFISLLLLFSSAYSRGVFRRDTHKSEIAHRFNDLGEENFQGLVLIAFSQYLQQCPFDEHVKLVKELTEFAKTCVADESHAGCDKSLHTLFGDELCKVATLRETYGDMADCCEKQEPERNECFLNHKDDSPDLPKLKPEPDTLCAEFKADEKKFWGKYLYEVARRHPYFYAPELLYYANKYNGVFQECCQAEDKGACLLPKIDAMREKVLASSARQRLRCASIQKFGERALKAWSVARLSQKFPKADFTDVTKIVTDLTKVHKECCHGDLLECADDRADLAKYICDHQDALSSKLKECCDKPVLEKSHCIAEVDKDAVPENLPPLTADFAEDKEVCKNYQEAKDVFLGSFLYEYSRRHPEYAVSVLLRLAKEYEATLEDCCAKEDPHACYATVFDKLKHLVDEPQNLIKKNCELFEKHGEYGFQNALIVRYTRKAPQVSTPTLVEISRSLGKVGTKCCAKPESERMPCTEDYLSLILNRLCVLHEKTPVSEKVTKCCTESLVNRRPCFSDLTLDETYVPKPFDEKFFTFHADICTLPDTEKQIKKQTALVELLKHKPKATDEQLKTVMENFVAFVDKCCAADDKEGCFVLEGPKLVASTQAALA

SEQ ID NO: 109 - protein, 381 aa, Sus scrofa
MPFGNTHNKYKLNFKAEEEYPDLSKHNNHMAKALTLEIYKKLRDKETPSGFTLDDVIQTGVDNPGHPFIMTVGCVAGDEESYVVFKDLFDPIIQDRHGGYKPTDKHKTDLNHENLKGGDDLDPNYVLSSRVRTGRSIKGYTLPPHCSRGERRAVEKLSVEALNSLTGEFKGKYYPLKSMTEQEQQQLIDDHFLFDKPVSPLLLASGMARDWPDARGIWHNDNKSFLVWVNEEDHLRVISMEKGGNMKEVFRRFCVGLQKIEEIFKKAGHPFMWNEHLGYVLTCPSNLGTGLRGGVHVKLAHLSKHPKFEEILTRLRLQKRGTGGVDTAAVGSVFDVSNADRLGSSEVEQVQLVVDGVKLMVEMEKKLEKGQSIDDMIPAQK

SEQ ID NO: 110 - protein, 902 aa, Sus scrofa
MMMVMQPEGLGTREGPFAGGSGGGGEYMEQEEDWDRDLLLDPAWEKQQRKTFTAWCNSHLRKAGTQIENIEEDFRNGLKLMLLLEVISGERLPRPDKGKMRFHKIANVNKALDFIASKGVKLVSIGAEEIVDGNLKMTLGMIWTIILRFAIQDISVEETSAKEGLLLWCQRKTAPYRNVNVQNFHTSWKDGLALCALIHRHRPDLIDYAKLRKDDPIGNLNTAFEVAEKYLDIPKMLDAEDIVNTPKPDEKAIMTYVSCFYHAFAGAEQAETAANRICKVLAVNQENEKLMEEYEKLASELLEWIRRTVPWLENRVGEPSMSAMQRKLEDFRDYRRLHKPPRVQEKCQLEINFNTLQTKLRLSHRPAFMPSEGKLVSDIANAWRGLEQVEKGYEDWLLSEIRRLQRLQHLAEKFQQKASLHEAWTRGKEDMLSQRDYESASLQEVRALLRRHEAFESDLAAHQDRVEHIAALAQELNELDYHEAASVNSRCQAICDQWDNLGTLTQKRRDALERMEKLLETIDQLQLEFARRAAPFNNWLDGAVEDLQDVWLVHSVEETQSLVTAHEQFKATLPEADRERGAILGIQGEIQKICQTYGLRPSSTNPYIALSPQDINTKWDTVRKLVPSRDQMLQEELTRQQVNERLRRQFAAQANAIGPWIQGKVEEVGRLAAGMAGSLEEQMAGLRQQEQNIINYKSNIDRLEGDHQLLQESLVFDNKHTVYSMEHIRVGWEQLLTSIARTINEVENQVLTRDAKGLSQEQLNEFRASFNHFDRKRNGMMEPDDFRACLISMGYDLGEVEFARIMTMVDPNAAGVVTFQAFIDFMTRETAETDTAEQVVASFKILAGDKNYITAEELRRELPAEQAEYCIRRMAPYKGAGAPAGALDYVAFSSALYGESDL

SEQ ID NO: 111 - protein, 844 aa, Sus scrofa
MAMSRPLTDQEKRKQISVRGLAGVENVAELKKNFNRHLHFTLVKDRNVANARDYYFALAYTVRDHLVGRWIRTQQHYYEKDPKRIYYLSLEFYMGRTLQNTMVNLALENACDEATYQLGLDMEELEEMEEDAGLGNGGLGRLAACFLDSMATLGLAAYGYGIRYEFGIFNQKITGGWQMEEADDWLRYGNAWEKARPEFTLPVHFYGRVEHTNQGAKWVDTQVVLAMPYDTPVPGYRNNVVNTMRLWSAKAPNDFNLKDFNVGGYIQAVLDRNLAENISRVLYPNDNFFEGKELRLKQEYFVVAATLQDIIRRFKSSKFGCRDPVRTSFDAFPDKVAIQLNDTHPSLAIPELMRILVDEERLEWEKAWDVTVRTCAYTNHTVLPEALERWPVHLMETLLPRHLQIIYEINQRFLNRVAAAYPGDVDRLRRMSLVEEGAVKRINMAHLCIAGSHAVNGVARIHSEILKKTIFKDFYELEPHKFQNKTNGITPRRWLVMCNPGLAEVIAERIGEEYIADLDQLRKLLSYVDDEAFIRDVAKVKQENKLKFSAYLEREYKVHINPNSLFDIQVKRIHEYKRQLLNCLHVITLYNRIKKEPNKFVVPRTVMIGGKAAPGYHMAKMIIKLITAIGDVVNHDPVVGDRLRVIFLENYRVSLAEKVIPAADLSEQISTAGTEASGTGNMKFMLNGALTIGTMDGANVEMAEEAGEENFFIFGMRVEDVERLDQKGYNAQEYYDRIPELRHVIDQLSSGFFSPKQPDLFKDIVNMLMHHDRFKVFADYEDYIKCQERVSALYKNPREWTRMVIRNIATSGKFSSDRTIAQYAREIWGVEPSRQRLPAPDEKI

SEQ ID NO: 112 - protein, 368 aa, Sus scrofa
MPHQYPALTPEQKKELSDIAHRIVAPGKGILAADESTGSIAKRLQSIGTENTEENRRFYRQLLLTADDRVNPCIGGVILFHETLYQKADDGRPFPQVIKAKGGVVGIKVDKGVVPLAGTNGETTTQGLDGLSERCAQYKKDGADFAKWRCVLKIGEHTPSSLAIMENANVLARYASICQQVGLQNGIVPIVEPEILPDGDHDLKRCQYVTEKVLAAVYKALSDHHIYLEGTLLKPNMVTPGHACTQKYSHEEIAMATVTALRRTVPPAVPGITFLSGGQSEEEASINLNAINKCPLLKPWALTFSYGRALQASALKAWGGKKENLKAAQEEYVKRALANSLACQGKYTPSGKAGAAASESLFISNHAY

SEQ ID NO: 113 - protein, 191 aa, Sus scrofa
MAPKKDVKKPAAAAAPAPAPAPAPAPAPAPPKEEKIDLSAIKSFSADQIAEFKEAFLLFDRTGECKITLSQVGDVLRALGTNPTNAEVKKVLGNPSNEEMNAKKIEFEQFLPMLQAISNNKDQGSYEDFVEGLRVFDKEGNGTVMGAELRHVLATLGEKMKEEEVEALMAGQEDSNGCINYEAFVKHIMSI

SEQ ID NO: 114 - protein, 333 aa, Sus scrofa
MVKVGVNGFGRIGRLVTRAAFNSGKVDIVAINDPFIDLHYMVYMFQYDSTHGKFHGTVKAENGKLVINGKAITIFQERDPANIKWGDAGATYVVESTGVFTTMEKAGAHLKGGAKRVIISAPSADAPMFVMGVNHEKYDNSLKIVSNASCTTNCLAPLAKVIHDHFGIVEGLMTTVHAITATQKTVDGPSGKLWRDGRGAAQNIIPASTGAAKAVGKVIPELNGKLTGMAFRVPTPNVSVVDLTCRLEKPAKYDDIKKVVKQASEGPLKGILGYTEDQVVSCDFNSDTHSSTFDAGAGIALNDHFVKLISWYDNEFGYSNRVVDLMVHMASKE

SEQ ID NO: 115 - protein, 284 aa, Sus scrofa
MDAIKKKMQMLKLDKENALDRAEQAEADKKAAEDRSKRLEDELVSLQKKLKATEDELDKYSEAPKDAQEKLELAEKKATDAEADVASLNRRIQLVEEELDRAQERLATALQKLEEAEKAADESERGMKVIESRAQKDEEKMEIQEIQLKEAKHIAEDADRKYEEVARKLVIIESDLERAEERAELSEGKCAELEEELKTVTNNLKSLEAQAEKYSQKEDKYEEEIKVLSDKLKEAETRAEFAERSVTKLEKSIDDLEDELYAQKLKYKAISEELDHALNDMTSI

SEQ ID NO: 116 - protein, 1220 aa, Sus scrofa
MPEAKPATKKAPAGKDAAKPAPKEAPPKGAPEEPPKEAPPEDQSPTAEEPTGIFLKKPDSVSVENGKDALIVAKVNGKELPGKPEIKWFKGKWLELGSKSGARFSFKESHDSASNVYTLELRIGKVVLGDRGDYRLEVKAKDFCDSCAFNIDVEAPRQDSAGQSLESFKRSGEGKPEGAGELDFSGLLKKRQVVEEEKKKKKDDDDLGIPPEIWELLKGAKKSEYEKIAFQYGITDLRGMLKRLKKAKVEVKKSAAFTKKLDPAYQVDQGNKIKLVVEISDPDLPLKWYKNGQEIKPSSKYVFENVGKKRILCINKCTLADDAAYEVAVKDEKCFTELFVREPPVLIVTPLEDQQVYVGDRVEMTVEVSEEGAQVMWLKDGVELTREDSFKSRYRFKKDGKRHILIYSDVSLEDRGRYQVMTNGGQCEAELSVEEKQLEVLQDIADLTVKASEQAVFKCEVSDEKVTGKWYKNGVEVRPSKRITISHVGRFHKLVIDDVRPEDEGDYTFVPDGYALSLSAKLNFLEIKVEYVPKQEPPKIHLDCSGKTSENAVVVVAGNKLRLDVSITGEPPPVATWMKEDEVFSGTEGRVRLEQRADISSFVIESAERGDEGRYTIKVTNPAGEDVASIFLRVVDVPDPPEAVRVTTVGEDWAILVWEPPKYDGGQPVTGYLLERKKKGSQRWMKLNFEVFTETTYESTNMIEGILYEMRVFAVNAIGVSQPSVNTKPFMPIAPTSEPQHLTVEDVTDTTTTLKWRPPDRIGAGGIDGYLVEYCVEGSDEWIPANTEPVERCGFTVKNLPTGARITFRVVGVNIAGRSQPATLAQPVTIREIVGEPPRQSSVLIRASPDSLVATTLSVPLRRVPQPYAEPAPGIVSPEQPKIRLPRHLRQTYIRKVGEHLNLVIPFQGKPRPQVVWTKGGAPIDTSRVHVRTSNFDTVFFVRQAARSDSGEYELSVQIENMKDTATVHIRVVEKAGPPQNVMVKEVWGTNALVEWQPPKDDGNTEITGYFVQKADKKTMEWFNVYEHNRHTSCTVSDLIMGNEYYFRVYSENICGLSDSPGVSKNTARILKTGITFKLPEFKEHDFRTPPKFLTPLPDRVVVAGYTAALNCAVRGHPKPKVVWMKNQMEIGEDPKFLMTNHQGVLTLNIRRPSPFDSGTYSCRASTSWGRPWPSASWKSECRSEAPASTQGSWLLSPDPDLPEPLSLSRVITRFLSRTQ

SEQ ID NO: 117 - protein, 869 aa, Sus scrofa
TMQHVYTKMSLVAVPFYQKRHRHFDQSYRNIQTRYLLDEYAAKKRASTQSSSQKSVTQESSSQRESSSERASSQTLLGGTTCRLCAKRVSTALEEEEQERKRRYHSLLAAYGEAKRERYLSELAQLEEDVHLARTHARDKLDRFALQHVDTHALRHAVGDRLAWERHSFEDRISRAPEILVRLRSHTVWERMSVKLCFTVQGFPTPVVQWYKDGSLICQAGEPGKYRIESKYGVHTLEINRADFDDTATYSAVATNVHGQVSTNAAVVVRRFHGDEEPFHSVGLPIRLPLSSVIPYTHFDVQFLEKFGVTFRREGETLTLKCTLLVTPDLKRVQPRAEWYRDDVLLKESKWTKMFFGEGQASLSFSHLNKDDEGLYTLRIVSRGGISDHSAFLFVRDADPLVMGAPGAPMDVHCHDANRDYVIVTWKPPNTTKESPVLGYFIDRCEVGTDNWVQCNDAPVKICKYPVSGLVEGRSYTFRVRAVNSAGVSRPSRASEAVAALDPLDLKRLQGIHWEGDKDIVIYQDDLEGEVQIPRPPTNVHASEISRTYVVLSWDPPTPRGKEPLMYFIEKSVVGSGTWQRENAQTAVRSPRYAVFDLAEGKSYVFRVLSANKHGLSDPSEITSPIQAQDTIVVPSAPGRVLASRNTKTSVVVQWDQPKHGEDLLGYYVDCCVAGSNVWEPCNHKPIGYNRFVVHGLTTGEQYIFRVKAVNAVGTSENSQESDVIKVQAALTVPSHPYGITLLNCDGHSMTLGWKVPKFSGGSPIIGYYVDKREGHHKNWHEVNSSPLKERILTVEGLTEGSTYEFKIAAPTWWGSGCRRIPVSSSSVRPGQRLSQVPTSGWVGTLGFFLVLLSLYRRYLLGSGEHGDS

SEQ ID NO: 118 - protein, 434 aa, Sus scrofa
MAMQKIFAREILDSRGNPTVEVDLHTAKGRFRAAVPSGASTGIYEALELRDGDKSRYLGKGVLKAVEHINKTLGPALLEKKLSVVDQEKVDKFMIELDGTENKSKFGANAILGVSLAVCKAGAAEKGVPLYRHIADLAGNPDLVLPVPAFNVINGGSHAGNKLAMQEFMILPVGASSFREAMRIGAEVYHHLKGVIKGKYGKDATNVGDEGGFAPNILENNEALELLKTAIQAAGYPDKVVIGMDVAASEFYRNGKYDLDFKSPDDPSRHITGEKLGELYKSFIKNYPVVSIEDPFDQDDWKTWTSFLSGVNIQIVGDDLTVTNLKRIAQAVEKKACNCLLLKVNQIGSVTESIQACKLAQSNGWGVMVSHRSGETEDTFIADLVVGLCTGQIKTGAPCRSERLAKYNQLMRIEEALGDKAVFAGRKFRNPKAK

SEQ ID NO: 119 - protein, 169 aa, Sus scrofa
MAPKKAKRRAAAEGSSNVFSMFDQTQIQEFKEAFTVIDQNRDGIIDKEDLRDTFAAMGRLNVKNEELDAMMKEASGPINFTVFLTMFGEKLKGADPEDVITGAFKVLDPEGKGTIKKHFLEELLTTQCDRFSQEEIKNMWAAFPPDVGGNVDYKNICYVITHGDAKDQE

SEQ ID NO: 120 - protein, 1939 aa, Sus scrofa
MSSDQEMAIFGEAAPYLRKSEKERIEAQNKPFDAKTSVFVAEPKESFVKGTVQSREGGKVTVKTEAGATLTVKEDQVFPMNPPKFDKIEDMAMMTHLHEPAVLYNLKERYAAWMIYTYSGLFCVTVNPYKWLPVYNAEVVTAYRGKKRQEAPPHIFSISDNAYQFMLTDRENQSILITGESGAGKTVNTKRVIQYFATIAVTGEKKKEEPTSGKMQGTLEDQIISANPLLEAFGNAKTVRNDNSSRFGKFIRIHFGTTGKLASADIETYLLEKSRVTFQLKAERSYHIFYQIMSNKKPELIEMLLITTNPYDYAFVSQGEITVPSIDDQEELMATDSAIEILGFTSDERVSIYKLTGAVMHYGNLKFKQKQREEQAEPDGTEVADKAAYLQGLNSADLLKALCYPRVKVGNEFVTKGQTVQQVYNAVGALAKAVYDKMFLWMVTRINQQLDTKQPRQYFIGVLDIAGFEIFDFNSLEQLCINFTNEKLQQFFNHHMFVLEQEEYKKEGIEWEFIDFGMDLAACIELIEKPMGIFSILEEECMFPKATDTSFKNKLYEQHLGKSNNFQKPKPAKGKVEAHFSLIHYAGTVDYNITGWLDKNKDPLNETVVGLYQKSSVKTLAFLFTGAAGADAEAGGGKKGGKKKGSSFQTVSALFRENLNKLMTNLRSTHPHFVRCIIPNETKTPGAMEHELVLHQLRCNGVLEGIRICRKGFPSRILYADFKQRYKVLNASAIPEGQFIDSKKASEKLLGSIDIDHTQYKFGHTKVFFKAGLLGLLEEMRDEKLAQLITRTQARCRGFLARVEYQKMVERRESIFCIQYNIRAFMNVKHWPWMKLYFKIKPLLKSAETEKEMANMKEEFEKTKESLAKAEAKRKELEEKMVALMQEKNDLQLQVQAEADSLADAEERCDQLIKTKIQLEAKIKEVTERAEDEEEINAELTAKKRKLEDECSELKKDIDDLELTLAKVEKEKHATENKVKNLTEEMAGLDETIAKLTKEKKALQEAHQQTLDDLQAEEDKVNTLTKAKTKLEQQVDDLEGSLEQEKKLRMDLERAKRKLEGDLKLAQESTMDIENDKQQLDEKLKKKEFEMSNLQSKIEDEQALAMQLQKKIKELQARIEELEEEIEAERASRAKAEKQRSDLSRELEEISERLEEAGGATSAQIEMNKKREAEFQKMRRDLEEATLQHEATAATLRKKHADSVAELGEQIDNLQRVKQKLEKEKSEMKMEIDDLASNMETVSKAKGNLEKMCRTLEDQLSELKTKEEEQQRLINDLTAQRARLQTESGEYSRQLDEKDTLVSQLSRGKQAFTQQIEELKRQLEEEIKAKSALAHAVQSSRHDCDLLREQYEEEQEAKAELQRAMSKANSEVAQWRTKYETDAIQRTEELEEAKKKLAQRLQDAEEHVEAVNAKCASLEKTKQRLQNEVEDLMIDVERSNAACAALDKKQRNFDKILAEWKQKYEETHAELEASQKESRSLSTELFKVKNAYEESLDQLETLKRENKNLQQEISDLTEQIAEGGKRIHELEKIKKQVEQEKSEIQAALEEAEASLEHEEGKILRIQLELNQVKSEVDRKIAEKDEEIDQLKRNHVRVVESMQSMLDAEIRSRNDAIRLKKKMEGDLNEMEIQLNHANRMAAEALRNYRNTQGILKDTQIHLDDALRSQEDLKEQLAMVERRANLLQAEIEELRATLEQTERSRKVAEQELLDASERVQLLHTQNTSLINTKKKLETDISQIQGEMEDIIQEARNAEEKAKKAITDAAMMAEELKKEQDTSAHLERMKKNLEQTVKDLQHRLDEAEQLALKGGKKQIQKLEARVRELEGEVESEQKRNVETVKGLRKHERRVKELTYQTEEDRKNILRLQDLVDKLQAKVKSYKRQAEEAEEQSNVNLSKFRKLQHELEEAEERADIAESQVNKLRVKSREVHTKIISEE

SEQ ID NO: 121 - protein, 6665 aa, Sus scrofa
MADDEEYEEVVEYYTEETVYEEVPGETITEVYETTTTRTSGYEQSDTSQPAVAQPMPSKPVERKKVIRKKVDPSKFMTPYIAHSQKMQDLFSTNKYKKNYEKAKGKPYAITTDTPELRRIKKVQDQLSEVKYRIDGDVAKTICHVDEKAKDIEHAKKVSQQVSKVLYKQNWEDTKDKYLLPPDAPELVQAIKNTAMFSKKLYTEDWEADKSLFYPYNDSPELRRVAQAQKALSDIAYKKGHAEQQTQFTSLPDPPDIEFAKKVTNQVSKQKYKEDYENKIKGKWSETPCFEIATARMNAKNISTRKYQEDFENMKDQIYFMQTETPEYKVNKQAGTAASKVKYKEDYEKNKGKADYNVLPASENPLLRHLKAAGDALSDKRYKENYEKTKAKSINYCETPKFKLDTVLHNFSSDTKYKDSYLKNILGHYVGSFEDPYHTHCMKVAAENSDKNYKAEYEEDRGKGFFPQTITQEYEAIKKLDQCKDHTYKVHPDKTKFTQVTDSPVLVQAQVNSKQLSDLNYKAKHENEKFKCHIPPDTPAFIQHKVNAYNLSDNVYKHDWEKTKAKKFDIKVDAIPLLAAKANTKNTSDVMYKKDYEKSKGKMIGALSINDDPKMLHSLKTAKNQSDRLYKENYEKTKAKSMNYCETPKYQLDTLLKNFSEAKYKDSYVQNVLGHYIGSFEDPYQLHCMKVSAQNSDKNYKAEYEEDKGKCYFPQTITQEYEAIKKLDQCKDHTYKVHPDKTKFTAVTDTPVLLQAQLNTKQLSDLNYKAKHEGEKFKCNIPADAPQFIQHRVNAYNLSDNVYKHDWEKTKAKKFDIKVDAIPLLAAKANTKNTSDVMYKKDYEKSKGKMIGALSINDDPKMLHSLKTAKNQSDREYRKDYEKSKTIYTAPLDMLQVTQAKKSQAIASDVDYKHLLHNYSYPPDSINVDLAKKAYALQSDVEYKADYNSWMKGCGWIPFGSLEMEKAKRASDILNEKKYRQHPETLKFTSIEDAPIIVQSKINQAQRSDIAYKAKGEEIIHKYSLPADLPFIQAKVNAYNISESMYKADLKDLSKKGYDLRTDAIPIKAAKAARQAASDVQYKKDYEKAKGKMVGFQSLQDDPKLVHYMNVAKIQSDREYKKDYEKTKTKYNTPHDMFNVVAAKKAQDVASNVNYRHSLHHYTYLPDAMDLELSKNMMHIQSDNAYKEDYNTWMKGIGWIPLGSLEVEKVKKAGDALNEKKYRQHPDTLKFTSIVDSPVMVQAKQNTRQVSDILYKAKGEDVKHKYTMGPDLPQFLQAKCNAYNISDVCYKRDWHDLIAKGNNVLGDAIPITAAKASRNIASDYKYKEAYEKAKGKHVGFRSLQDDPKLVHYMNVAKLQSDREYKKNYENTKTSYHTPGDMVSITAAKMAQDVATNVNYKQPIHHYTYLPDALSLEHTRNVNQIQSDNVYKDEYNHFFKGIGWIPLGSLEVEKVKKAGDALNERKYRQHPDTIKFTSVPDSMGMVLAQHNTKQLSDLNYKVEGEKLKHKYTMDPDVPQFIQAKVNAINMSDAHYKADWKKTLAKGYDLTPDAIPIVAAKSSRNIASDFKYKEAYEKAKGKQVGFRSLQDDPKLVHYMNVAKIQSDREYKKGYEASKTRYHTPLDMLSVTAAKKSQEVATNTNYKQPFHHYTLLPDALNVEHSRNAMQIQSDNLYKSDFTNWMKGIGWIPMESLEVEKAKKAGEILSEKKYRQHPEKLKFTYAMDTMEQALNKSNKLIMDKRLYTEKWNKDKTTIHVMPDTPDILLSRVNQITLSDKLYKAGWEEEKKKGYDLRPDAISIKAARASRDIASDYKYKQAYEQAKGKHIGFRSLEDDPKLVHFMQVAKMQSDREYKKAYEKSKTSFHTPVDMLSVVAAKKSQEVATNANYRNVIHTYNMLPDAMSFELAKNMMQLQSDNQYKADYADFMKGIGWLPLGSLEAEKNKKAMEIISEKKYRQHPDTLKYSTLMDSMNMVLAKNNAKIMNEHLYKQAWEADKTKVHIMPDIPQIILAKANAVNMSDKLYKLSLEESKKKGYDLRTDAISIQAAKASRDIASDYKYKHSYEKERGKMVGFRSLEDDPKLVHSMQVAKMQSDREYKKSYEKTKTSYHTPADMLSVTAARDAQANITNTNYKHLIHKYILLPDAMNIQLSKNMNHIQSDNEYKQDYNECYKGLGWSPAGSLEVEKAKKATEYASDQKYRQHPSNFQFQKLNDSMDMVLAKQNAHTMNKYLYTADWNKDKTKIHVMPDTPDILQAKQNQTLFSQKLYKLGWEEALKKGYDLPVDAISVQLAKVSRDNASDFKYKQGYRKQLGHHIGFRNLQDDPKLVLSMNVAKMQSEREYKKDFEKWKTKFTSPVDMLGVVLAKKCQALVSDVDYKNYLHQWTCLPDQNDVIQAKRSYELQSENLYKSDLEWLRGIGWSPLGSLEAEKNKRASEIISEKKYRQPPDRNKFTSIPDAMDIVLAKTNAKNRSDRLYREAWDKDKTQVHIMPDTPDIILAKANLINSSDKLYRMGYEELKKKGYDLPVDAIPIKAAKASREIASEYKYKEGFRKQLGHHVGARNIEDDPKMMWSMHVAKIQSDREYKKDFEKWKTKFSSPVDMLGVVLAKKCQTLVSDIDYKNYLHQWTCLPDQSDVIHARRAYDLQSDNLYKSDLQWLKGIGWLPSGSLEDEKNKRATQILSDHVYRQHPDKFKFSSLMDSIPMVLAKNNAITMNHRLYTEAWDKDKTTVHIMPDTPEVLLAKQNKINYSEKLYKLGLEEAKRKGYDMRLDAIPIRAAKASRDIASEFKYKEGYRKQLGHHIGARAIHDDPKMMWSMHVAKVQSDREYKKDFEKWKTKFSSPVDMLGVVLAKKCQTLVSDIDYKNYLHQWTCLPDQSDVIHARRAYDLQSDNMYKSDLQWLRGIGWVPIGSLDVEKCKRATEILSDKIYRQPPDTFKFTSVPDSLEQVLAKNNAINMNKRLYTEAWDKDKTQVHIMPDTPEIMLARQNRLNYSETLYKLANEEAKKKGYDLRSDAIPIVAAKASRDIVSDYKYKDGYRKQLGHHIGARNIEDDPKMMWSMHVAKIQSDREYKKDFEKWKTKFSSPVDMLGLVQAKKCQILVSDVDYKNYLHQWTCLPDQNDVIHARQAYDLQSDNIYKSDLQWLKGIGWVPIGSVDVVKCKRAAEILSDHIYRQPPDKLKFTSVPDSLEQVLAKNNAINMNKRLYTEAWDKDKTQIHIMPDTPEIMLARQNKINYSESLYRQAMEEAKKEGYDLRSDAIPIVAAKASRDIASDYKYKEAYRKQLGHHIGARAVHDDPKMMWSLHVAKMQSDREYKKDFEKYKTRFSSPVDMLGIVLAKKCQTLVSDVDYKHPLHEWTCLPDQNDIIHARKAYDLQSDNLYKSDLEWMKGIGWIPLGSVEVVKAKRAGEILSDNIYRQRPDTLKFTSVTDSPELVLAKNNALNMNKRLYTEAWDNDKKTIHVMPDTPEIMLAKLNRINYSDKLYRLALEESKKEGCDLRVDAIPIQAAKASREIASDYKYKEAYRKQLGHHIGARNIEDDPKMMWSIHAAKIQSDREYKKDFEKWKTRFSSPVDMLGLVQAKKCQILVSDIDYKHPLHEWICLPDQNDIIQARKAYDLQSDAIYKADLEWLRGIGWVPLGSVEVEKVKRAGEILSERKYRQPADQLKFTCITDTPEIVLAKNNALTMSKHLYTEAWDTDKTSIHVMPDTPEILLAKSNSANISQKLYTKGWDESKMKDYDLKADAISIKSAKASRDIASDYKYKEAYEKQKGHHIGAQSVEDDPKIMCAIHAGKIQSEREYKKDFQKWKTKFTSPVDMLGILLAKKCQTLVSDIDYRNYLHHWTCLPDQNDIIQARKAYELQSDALYKADLEWLRGIGWMPQGSPEVLRVKNAQEILRDSVYRTPVVKLKYTSIVDTPEVVLAKSNAENISIPKYREVWDKDKTSVHIMPDTPEINLARANALNVSNKIYREGWDEMKMSCDVRLDAIPIQAAKASREIASDYKYKLDHEKQKGHYVGTLTARDDNKIRWALIAGKIQNEREYRLHWAKWKSKFQSPADMLSITHSKNCQALVSDIDYRNYLHQWTCLPDQHDVIQARKAYDLQSDAIYKADLEWLRGIGWMPSDSVSVNHAKHAADIFSEKKYRTKIETLNFTPVDDRVDYVTAKQSGEILNDIKYRKDWNDTKSKYTLTETPQLHAAQEAARILDQYLYKESWEKQKATGYILPPDAVPFVHAHHSSDVQSELKYKADHVKQKGHYVGVPTMRDDPKLVWFEHAGQIQNDRLYKEHYHKTKAKINIPPDMVSVVAAKEGQILVSDIDYRNYLHQWTCHPDQNDVIQARKAYDLQSDNVYKADLEWLRGIGWIPLDSVDHVRVTRNQEMVNQIKYKKDALANYPNFTSVVDPPAVVLAKINSVNQSDVKYKETFNKLLKGKYIFSPDTPYITHSKDMGKLYSTILYKGAWEGTKAYGYTLDERYIPIVGAKHADFVNSELKYKETYEKQKGHYLAGKEISEFPNVVHCLDFQKMRSVLNYRRHYEDTKANVHIPNDMMNHVLAKRCQYILSDLEYRHYFHQWTSLPEEPNVIRARNAQEILSDNVYKDDLNWLKGIGCYVWDTPQILHAKKSYDLQSQILYTADGKENLKNYNMVTDTPLYVTALQSGINASEVKYKENYHQIKDKYTTVLETVDYDRTRNLKDLYSSNVYKEAWDKVKATSYILPSNTVSLTHAKNQKHLASHIKYREEYEKFKALYTLPKSVDDDPNTARCLRVGKFNIDRLYRSVYEKNKMKIHMVPDMVEMVTAKDSQKKVSEIDYRLHLHEWICHPDLQVNSHVRKVTDQISDIVYKDDLNWLKGIGCYVWDTPEILHAKHAYDLRNDIKYQAHVKKTRNNYKLVTDTPLYVQAVKSGKQLSDAVYHYDYVHSVRGKVAPTTKTVDLDRALHAYKLQSENLYRKDGLNSLPTGYRLPGDTPHFKHNKDIRYMSSYFKYKEVYEHFKANGYTLGPKDVPFVHVRRVNNVTSERLYRQLYHKLKDKIHTTPDTPEIRQVKKTQEAVSELIYKSDFFKMQGHMISMPYTPHVLHCRYVGDITSDIKYKEDLQILRGLGCFLYDTPDMVRSRHLRKLWSNYLYTDNARKMRDKYKVVLDTPEYRKVQELKTHLSELVYRAAGKKQKSIFTSVPDTPDLLRAKRGQKLQSQYLYVELATKERPHHHAGNQTTALKHARDVKDMVSEKKYKVQYEKMKDKYTPVPDTPILIRAKKAYWNASDLRYKETFQKTKGKYHTVKDALDIVYHRKVTDHISKIKYKENYMSQLGIWRSIPDRPEHFHHRAVTDAVSDVKYKEDLTWLKGIGCYAYDTPDFTLAEKNKTLYSKYKYKEVFERTKSDFKYEADCPINRHFKFATQLMNEKKYRADYEQRKDKYQLVVDEPRHLLAKIAGDQISQIKYRKNYEKTKDKFTSVVDTPEHLRTTKVNKQISDILYKLEYNKAKPRGYTTIHDTPMLLHVRKVKDEVSDLKYKEVYQRTKSNCTIEPDAVHIKAAKDAYKVNTNLDYKKQYEANKAHWRWTPDRPDFIQAAKSSLQQSDFEYKLDREFLKGCKLSVTDDKDMVLALRNSVIESDLKYKEKHVKERGTCHAVPDTPQILLAKTVSNLVSENKYKRHVKKHLAQGSYTTLPETRDTVHVKEVTKHVSDTNYKKKFVKERGKSNYSIMLEPPEVKHAMEVAKKQSDVAYKKDAKENLHYTTVADRPDIKKATQAAKQASEVEYRAKHRKEGSHGLSMLGRPDIEMAKKAATLSSQVKYRENFNKEKGKTPKYNPKESQLYKIMKDANNLASEVKYKADLKKLHKPVTDMKESLIMNHVLNTSHLASSYQYKKNYEKSKGHYHTIPDNLEQLHLKEATELQSIVKYKEKYEKERGKPMLDFETPTYITAKESQQMQSGKEYRKDYEESIKGRNLTGLEVTPALLHVKYATKIASEKEYRKDLEESIRGKGLSEMEDTPDMLRAKNATQILNEKEYKRDLELEVKGRGLNAMANETPDFMRARNATDIASQIKYKQLAEMEKANFTSVVDTPEIIHAQQVKNLSSQKKYKEDAEKCMSYYETVLDTPEMQRVRENQKNFSLLQYQCDLKNSKGKITVVQDTPEILRVKENQKNFSSVLYKEDVSPGTAIGKTPEMMRVKQTQDHISSVKYKETIGQGTPIPDLPEVKRVKETQKHISSVMYKENLGTGIPTPVTPEIERVKRNQENFSSVLYKENLGKGTPTPITPEMERVKRNQENFSSVLYKENMGKGTPLPVTPEMERVKHNQENISSILYKENVGKGTPTPVTPEMQRVKRNQENISSVIYKENLGKATPTPFTPEMERVKRNQENFSSVLYKENMRKATPTPVTPEMERAKRNQENISSVLYSDSFRKQIQGKAAYVLDTPEMRRVRETQRHISTVKYHEDFEKHKGCFTPVVTDPITERVKKNTQDFSDINYRGIQRKVVEMEQKRNDQDQETITGLRVWRTNPGSVFDYDPAEDNIQSRSLHMLNAQAQRRSREQSRSASALSISGGEEKSEHSEAARLSTYSDGGVFFSATSAAYKHAKTTELPQQRSSSVATQQTTVSSIPSHPSTAGKIFRAMYDYMAADADEVSFRDGDAIVNVQAIDEGWMYGTVQRTGRTGMLPANYVEAI

SEQ ID NO: 122 - protein, 1937 aa, Sus scrofa
MSSDQEMAIFGEAAPYLRKSEKERIEAQNKPFDAKTSVFVAEPKESFVKGTVQSREGGKVTVKTEAGATLTVKEDQVFPMNPPKFDKIEDMAMMTHLHEPAVLYNLKERYAAWMIYTYSGLFCVTVNPYKWLPVYNAEVVTAYRGKKRQEAPPHIFSISDNAYQFMLTDRENQSILITGESGAGKTVNTKRVIQYFATIAVTGEKKKEEPTPGKMQGTLEDQIISANPLLEAFGNAKTVRNDNSSRFGKFIRIHFGTTGKLASADIETYLLEKSRVTFQLKAERSYHIFYQIMSNKKPELIEMLLITTNPYDYAFVSQGEITVPSIDDQEELMATDSAIEILGFTSDERVSIYKLTGAVMHYGNLKFKQKQREEQAEPDGTEVADKAAYLQGLNSADLLKALCYPRVKVGNEFVTKGQTVQQVYNAVGALAKAVYDKMFLWMVTRINQQLDTKQPRQYFIGVLDIAGFEIFDFNSLEQLCINFTNEKLQQFFNHHMFVLEQEEYKKEGIEWEFIDFGMDLAACIELIEKPMGIFSILEEECMFPKATDTSFKNKLYEQHLGKSNNFQKPKPAKGKAEAHFSLIHYAGTVDYNITGWLDKNKDPLNETVVGLYQKSSVKTLAFLFAERQSSEEGGTKKGGKKKGSSFQTVSALFRENLNKLMTNLRSTHPHFVRCIIPNETKTPGAMEHELVLHQLRCNGVLEGIRICRKGFPSRILYADFKQRYKVLNASAIPEGQFIDSKKASEKLLGSIDIDHTQYKFGHTKVFFKAGLLGTLEEMRDEKLAQLITRTQAMCRGFLMRVEFRKMMERRESIFCIQYNIRAFMNVKHWPWMKLYFKIKPLLKSAETEKEMANMKEEFEKTKEDLAKSEAKRKELEEKMVALMQEKNDLQLQVQAEADGLADAEERCDQLIKTKIQLEAKIKEVTERAEDEEEINAELTAKKRKLEDECSELKKDIDDLELTLAKVEKEKHATENKVKNLTEEMAGLDENIAKLTKEKKALQEAHQQTLDDLQAEEDKVNTLTKAKTKLEQQVDDLEGSLEQEKKLRMDLERAKRKLEGDLKLAQESTMDIENDKQQLDEKLKKKEFEMSNLQSKIEDEQALAMQLQKKIKELQARTEELEEEIEAERASRAKAEKQRSDLSRELEEISERLEEAGGATSAQIEMNKKREAEFQKMRRDLEEATLQHEATAAALRKKHADSVAELGEQIDNLQRVKQKLEKEKSELKMEIDDLASNMETVSKAKGNLEKMCRTLEDQLSEVKTKEEEHQRLINELSAQKARLQTESGEFSRQLDEKEALVSQLSRGKQAFTQQIEELKRQLEEETKAKSALAHAVQSSRHDCDLLREQYEEEQEAKAELQRAMSKANSEVAQWRTKYETDAIQRTEELEEAKKKLAQRLQDAEEHVEAVNAKCASLEKTKQRLQNEVEDLMLDVERSNAACAALDKKQRNFDKILAEWKHKYEETQAELEASQKESRSLSTELFKVKNAYEESLDQLETLKRENKNLQQEISDLTEQIAEGGKHIHELEKVKKQIEQEKSELQAALEEAEASLEHEEGKILRIQLELNQVKSEIDRKIAEKDEEIDQMKRNHIRVVESMQSTLDAEIRSRNDALRIKKKMEGDLNEMEIQLNHANRQATEAIRNLRNTQGVLKDTQLHLDDAIRGQDDLKEQLAMVERRANLMQAEIEELRASLEQTERSRRVAEQELLDASERVQLLHTQNTSLINTKKKLETDISQIQGEMEDIVQEARNAEEKAKKAITDAAMMAEELKKEQDTSAHLERMKKNMEQTVKDLQHRLDEAEQLALKGGKKQIQKLEARVRELENEVENEQKRNVEAVKGLRKHERRVKELTYQTEEDRKNVLRLQDLVDKLQSKVKAYKRQAEEAEEQSNVNLSKFRKLQHELEEAEERADIAESQVNKLRVKSREVHTKVISEE

SEQ ID NO: 123 - protein, 607 aa, Sus scrofa
MKWVTFISLLFLFSSAYSRGVFRRDTYKSEIAHRFKDLGEQYFKGLVLIAFSQHLQQCPYEEHVKLVREVTEFAKTCVADESAENCDKSIHTLFGDKLCAIPSLREHYGDLADCCEKEEPERNECFLQHKNDNPDIPKLKPDPVALCADFQEDEQKFWGKYLYEIARRHPYFYAPELLYYAIIYKDVFSECCQAADKAACLLPKIEHLREKVLTSAAKQRLKCASIQKFGERAFKAWSLARLSQRFPKADFTEISKIVTDLAKVHKECCHGDLLECADDRADLAKYICENQDTISTKLKECCDKPLLEKSHCIAEAKRDELPADLNPLEHDFVEDKEVCKNYKEAKHVFLGTFLYEYSRRHPDYSVSLLLRIAKIYEATLEDCCAKEDPPACYATVFDKFQPLVDEPKNLIKQNCELFEKLGEYGFQNALIVRYTKKVPQVSTPTLVEVARKLGLVGSRCCKRPEEERLSCAEDYLSLVLNRLCVLHEKTPVSEKVTKCCTESLVNRRPCFSALTPDETYKPKEFVEGTFTFHADLCTLPEDEKQIKKQTALVELLKHKPHATEEQLRTVLGNFAAFVQKCCAAPDHEACFAVEGPKFVIEIRGILA

SEQ ID NO: 124 - protein, 381 aa, Bison bison; identical to SEQ ID NO: 79

SEQ ID NO: 125 - protein, 894 aa, Bison bison
MNQIEPGVQYNYVYEDDEYMIQEEEWDRDLLLDPAWEKQQRKTFTAWCNSHLRKAGTQIENIEEDFRNGLKLMLLLEVISGERLPKPDRGKMRFHKIANVNKALDYIASKGVKLVSIGAEEIVDGNVKMTLGMIWTIILRFAIQDISVEETSAKEGLLLWCQRKTAPYRNVNIQNFHTSWKDGLGLCALIHRHRPDLIDYSKLNKDDPIGNINLAMEIAEKHLDIPKMLDAEDIVNTPKPDERAIMTYVSCFYHAFAGAEQAETAANRICKVLAVNQENERLMEEYERLASELLEWIRRTIPWLENRTPEKTMQAMQKKLEDFRDYRRKHKPPKVQEKCQLEINFNTLQTKLRISNRPAFMPSEGKMVSDIAGAWQRLEQAEKGYEEWLLNEIRRLERVEHLAEKFRQKASTHETWAYGKEQILLQKDYESSTLTEVRALLRKHEAFESDLAAHQDRVEQIAAIAQELNELDYHDAVNVNDRCQKICDQWDRLGTLTQKRREALERTEKLLETIDQLHLEFAKRAAPFNNWMEGAMEDLQDMFIVHSIEEIQSLITAHEQFKATLPEADGERQSILAIQNEVEKVIQSYSIRISSSNPYSTVTVDEIRSKWDKVKQLVPIRDQSLQEELARQHANERLRRQFAAQANAIGPWIQNKMEEIARSSIQITGALEDQMNQLKQYEHNIINYKNNIDKLEGDHQLIQEALVFDNKHTNYTMEHIRVGWELLLTTIARTINEVETQILTRDAKGITQEQMNEFRASFNHFDRRKNGLMDHEDFRACLISMGYDLGEAEFARIMTLVDPNGQGTVTFQSFIDFMTRETADTDTAEQVIASFRILASDKPYILAEELRRELPPDQAQYCIKRMPAYSGPGSVPGALDYTAFSSALYGESDL

SEQ ID NO: 126 - protein, 842 aa, Bison bison
MSRPLTDQEKRKQISVRGLAGVENVTELKKNFNRHLHFTLVKDRNVATPRDYYFALAYTVRDHLVGRWIRTQQHYYEKDPKRIYYLSLEFYIGRTLQNTMVNLALENACDEATYQLGLDMEELEEIEEDAGLGNGGLGRLAACFLDSMATLGLAAYGYGIRYEFGIFNQKISGGWQMEEADDWLRYGNPWEKARPEFTLPVHFYGRVEHTSQGAKWVDTQVVLAMPYDTPVPGYRNNVVNTMRLWSAKAPNDFNLKDFNVGGYIQAVLDRNLAENISRVLYPNDNFFEGKELRLKQEYFVVAATLQDIIRRFKSSKFGCLDPVRTNFDAFPDKVAIQLNDTHPSLAIPELMRILVDQERLEWEKAWEVTVKTCAYTNHTVLPEALERWPVHLIETLLPRHLQIIYEINQRFLNRVAAAFPGDVDRLRRMSLVEEGAVKRINMAHLCIAGSHAVNGVARIHSEILKKTIFKDFYELEPHKFQNKTNGITPRRWLVMCNPGLAEIIAERIGEEYIADLDQLRKLLSYVDDESFIRDVAKVKQENKLKFSAYLEKEYKVHINPNSLFDIQVKRIHEYKRQLLNCLHVITLYNRIKKEPNKFFVPRTVMIGGKAAPGYHMAKMIIKLITAIGDVVNHDPVVGDRLRVIFLENYRVSLAEKVIPAADLSEQISTAGTEASGTGNMKFMLNGALTIGTMDGANVEMAEEAGEENFFIFGMRVEDVERLDQKGYNAQEYYDRIPELRHIIDQLSSGFFSPKQPDLFKDIVNMLMHHDRFKVFADYEEYVKCQERVSALYKNPREWTRMVIRNIATSGKFSSDRTIAQYAREIWGVEPTRQRMPAPDEKI

SEQ ID NO: 127 - protein, 364 aa, Bison bison; identical to SEQ ID NO: 82

SEQ ID NO: 128 - protein, 192 aa, Bison bison
MAPKKDVKNPAAAAAPAPAPAPAPAPAPAPPKEEKIDLSAIKIEFSKQQQDEFKEAFLLFDRTGECKITLSQVGDVLRALGTNPTNAEVKKVLGNPSNEEMNAKKIEFEQFLPMLQAISNNKDQGTYEDFVEGLRVFDKEGNGTVMGAELRHVLATLGEKMKEEEVEALMAGQEDSNGCINYEAFVKHIMSN

SEQ ID NO: 129 - protein, 333 aa, Bison bison
  VARIANT 158: note = Any amino acid
  VARIANT 290: note = Any amino acid
MVKVGVNGFGHIGRLVTRAAFNSGKVDVIAINDPFIDLHYIVYMFQYDTTHGKFHGTVKAENGKLIINGKAVTIFQERDPASIKWGDAGAEYVVESTGVFTTMEKAGAHLKGGAKSVIISAPSADAPMFVLGMNHEKYNNNLKIVSNASCTTNCLAPXAKVIHDHFGIVEGLMTTFHAITVTQKTVDGPSGKLWHDGGGAAQNIIPASTGAAKAVGKVIPELNRKLTGMAFCVPTPNVSVMDLTYHLEKPAKYDEIKKVVKQTSEGPLEGILGYTQDQVVSCDFNSDSYXSTFDAGAGIALNDHFVKLISWYDNEFGYSNRVVDLMVHMASKE

SEQ ID NO: 130 - protein, 284 aa, Bison bison
  VARIANT 30: note = Any amino acid
MDAIKKKMQMLKLDKENALDRAEQAEADKXPVPHGHSQLEDELVSLQKKLKATEDELDKYSEALKDAQEKLELAEKKATDAEADVASLNRRIQLVEEELDRAQERLATALQKLEEAEKAADESERGMKVIESRAQKDEEKMEIQEIQLKEAKHIAEDADRKYEEVARKLVIIESDLERAEERAELSEGKCAELEEELKTVTNNLKSLEAQAEKYSQKEDKYEEEIKVLSDKLKEAETRAEFAERSVTKLEKSIDDLEDELYAQKLKYKAISEELDHALNDMTSM

SEQ ID NO: 131 - protein, 1183 aa, Bison bison
MPEAKPAAKKAPAGKDAAAKPAPKEATPQEAPAAPPTGEIDGAPAGRAEPGGSSAASRVVPPRPLLRSVFPTAPGLTALPYSQPCRAPQRFQGGGLTVPLPPHSPLLPGKDTVIVAKLNGKELPAKPAVKWFKGKWLELGSKSGARFSFKESHDAASNVYTIELHITKVVLGDRGDYRIEVKAKDFCDSCAFNIDVEAPRQDSAGQSLESFKRSGEAKSDTAGELDFSGLLKKRQVVEEEKKKKKDDDDLGIPPEIWELLKGAKKSEYERIAFQYGITDLRGMLKRLKKAKVEVKKSAAFTKKLDPAYQVDRGNKIKLVVEISDPDLPLKWFKNGQEIKPSSKYVFENVGKKRILTINKCTLADDAAYEVVVKDEKCFTELFVKEPPVLIVTPLEDQQVFVGDRVEMAVEVSEEGAQVMWLKDGVELTREDSFKARYRLKKDGKRHILIYSEVTMEDKGHYQVMTNGGQCEAELIVEEKQLEVLQDIADLTVKASEQAVFKCEVSDEKVTGKWYKNGVEVRPSKRITISHTGRFHKLVIDDVRPEDEGDYTFVPDGYALSLSAKLKFLEIKVEYVPKQEPPKIHLDCSGQTSENAIVVVAGNKLRMDVSITGEPRPVATWMKEDEVFTGTEGRVHIEQRGDISSFVIESAERGDEGRYTIKVTNPVGEDVASILLKVVDVPDPPEAVRVTSVGEDWAVLVWEPPKYDGGRPVTGYLLERKKKGSQRWMKLNFEVFTETTYESTNMIEGILYEMRVFAVNVIGVSQPSMNTKPFMPIAPTSEPRHLTVEDVTDTTTTLKWRPPDKIGAGGIDGYLIEYCVEGSDEWIPANTEPVERCGFTVKNLPTGAKITFRVVGVNIAGRSQPATLAQPVTIREIVEQPKIRLPRHLRQTYVRKVGEHLNLVIPFQGKPRPQVVWTKGGAPIDTSHVHVRTSDFDTVFFVRQAARSDSGEYELTVQIENMKDTATVCIRVVEKAGPPQNVMVKEVWGTNALVEWQPPKDDGNSEITGYFVQKADKKTMEWFTVYERNRHTSCTVSDLIMGNEYYFRVYSENVCGLSDLPGVSKNTARIVKTGMTLKLPEYKEHDFRTPPKFLTPLPDRVVVAGYAAALNCAVRGHPKPKVVWMKNKMEIREDPKFLMTNQQGVLTLNIRRPSPFDSGTYSCRAVNELGEALAECKLEVRVPQ

SEQ ID NO: 132 - protein, 1430 aa, Bison bison
MSLVAVPFYQKRHKHFDQSYRNIQTRYLLDEYSAKKKRASAQSSLQRSVTRKSSSQRESSRAALGESTLGETTLGETSLGDTTLGRTTCRLCAKRMSSALEEEAQERKRRYQSMVAAYGEILVRLRSHTVWERMSVKLCFTVQGFPTPVVQWYKDGSLICQAGEPGKYRIDSKYGVHTLEINRADFDDTATYSAVATNVHGQVSTNAAVVVRRFRGDEEPFHSVGLPIGLPLSSVIPYTHFDVQFIEKFGVTFRREGETLTLKCTLLVTPDLKRVQPRAEWYRDDVLLKESKWTKMFFGEGQASLSFSHLNKDDEGLYTLRIVSRGGITDHSAFLFVRDADPLVTGAPGAPMDVHCHDANRDYVIVTWKPPNTTKESPVIGYFIDRCEVGTDNWIQCNDAPVKICKYPVTGLFEGRSYIFRMRAVNSAGISRPSRVSEAVAALDPVDLRRLQAVHLEGEKDIVIYQEELEGEVQIPGPPTNVHASETSRTYVVLSWDPPEPRGKEPLMYFIEKSMVGSGSWQRVNAQTAVRSPRYAVFDLAEGKPYVFRVLSANKHGLSDPSEITAPIQAQDTIVVPSAPGRVLASRNTRTSVVVQWDRPKHEEDLLGYYVDCSVAGSNVGEPCNHKPIGYNRFVVHGLTTGEQYIFRVKAVNAVGTSENSQESDVIKVQAALTVPSHPYGITLLNCDGHSMILGWKVPKFSGGSPILGYYVDKREAHHKNWHEVNSSPLKERILTVEGLTEGSLYEFKIAAANMAGIGQPSDPSELFKCEAWTMPEPGPAYDLTFCEVRDTSLVLLWKPPVYSGSSPVSGYFVDYKEEDSGEWLTVHETATPHRYLKVCDLHQGKTYVFRVRAVNASGVGRPSDTSEPVLVEARPGTKEVSAGVDEEGNVYLSFDCPEVTDASHFTWCKSYEDIADDDRFKVETAGDHSKLYFKNLDKEDLGTYSVSVSDTDGVSSSFVLDEEELERLKALSNEIKNPTIPLKSELAYEIFDKGQVRFWLQAEHLSPDSSYRFIINDREVADSETHRIKCDKSTGIIEMVMDRFTIDNEGTYTVQIQDGKAKSQSSLVLIGDAFKAVLKEAEFQRKEFLRKQGPHFAEYLHWDVTEECEVRLVCKVANTKKETVFKWLKDDVLYETEKMPDLEKGVCELLIPKLSKKDHGEYKATLKDDRGQDVSTLEIAGKVYEDMILAMSRVCGASASPLKILCTPEGIRLQCFMKYFTEEMKVNWYHKDAKITSSEHMRIGGSEQMAWLQICEPTEKDKGKYTFEILDDKKNHQRSLDLSGQAFDEAFAEFQQLKAAAFAEKNRGKVIGGLPDVVTIMEGKTLNLTCTVFGNPDPEVVWFKNDKDIELSDHFSVKVEQAKYVSMTIKGVTSEDSGKYSIHVKNKYGGEKIDVTVSVYKHGEEIPDVVLPQQAKPKLLPAAAPSPAH

SEQ ID NO: 133 - protein, 440 aa, Bison bison
MRTLRAMAMQKIFAREILDSRGNPTVEVDLHTAKGRFRAAVPSGASTGIYEALELRDGDKSRYLGKGVLKAVEHINKTLGPALLEKKLSVVDQEKVDKFMIELDGTENKSKFGANAILGVSLAVCKAGAAEKGVPLYRHIADLAGNPELILPVPAFNVINGGSHAGNKLAMQEFMILPVGASSFREAMRIGAEVYHHLKGVIKAKYGKDATNVGDEGGFAPNILENNEALELLKTAIQAAGYPDKVVIGMDVAASEFYRNGKYDLDFKSPDDPARHISGEKLGELYKNFIKNYPVVSIEDPFDQDDWATWTSFLSGVNIQIVGDDLTVTNPKRIAQAVEKKACNCLLLKVNQIGSVTESIQACKLAQSNGWGVMVSHRSGETEDTFIADLVVGLCTGQIKTGAPCRSERLAKYNQLMRIEEALGDKAVFAGRKFRNPKAK

SEQ ID NO: 134 - protein, 170 aa, Bison bison
MAPKKAKRRAAAEGGSSSVFSMFDQTQIQEFKEAFTVIDQNRDGIIDKEDLRDTFAAMGRLNVKNEELDAMMKEASGPINFTVFLNMFGEKLKGADPEDVITGAFKVLDPEGKGTIKKQFLEELLTTQCDRFSHEEIKNMWAAFPPDVGGNVDYKNICYVITHGDAKDQE

SEQ ID NO: 135 - protein, 1938 aa, Bison bison
MSSDQEMAIFGEAAPYLRKSEKERIEAQNKPFDAKTSVFVADPKESFVKATVQSREGGKVTAKTEAGATVTVKEDQVFPMNPPKFDKIEDMAMMTHLHEPAVLYNLKERYAAWMIYTYSGLFCVTVNPYKWLPVYNAEVVTAYRGKKRQEAPPHIFSISDNAYQFMLTDRENQSILITGESGAGKTVNTKRVIQYFATIAVTGEKKKEEPTSGKMQGTLEDQIISANPLLEAFGNAKTVRNDNSSRFGKFIRIHFGTTGKLASADIETYLLEKSRVTFQLKAERSYHIFYQIMSNKKPELIEMLLITTNPYDYAYVSQGEITVPSIDDQEELMATDSAIEILGFTSDERVSIYKLTGAVMHYGNLKFKQKQREEQAEPDGTEVADKAAYLQGLNSADLLKALCYPRVKVGNEFVTKGQTVEQVYNAVGALAKAVYDKMFLWMVARINQQLDTKQPRQYFIGVLDIAGFEIFDFNSLEQLCINFTNEKLQQFFNHHMFVLEQEEYKKEGIEWEFIDFGMDLAACIELIEKPMGIFSILEEECMFPKATDMSFKNKLYEQHLGKSNNFQKPKPAKGKAEAHFSLIHYAGTVDYNITGWLDKNKDPLNETVVGLYQKSSVKTLALLFSGPASGEAEGGPKKGGKKKGSSFQTVSALFRENLNKLMTNLRSTHPHFVRCIIPNETKTPGAMEHELVLHQLRCNGVLEGIRICRKGFPSRILYADFKQRYKVLNASAIPEGQFIDSKKASEKLLASIDVDHTQYKFGHTKVFFKAGLLGLLEEMRDEKLAQLITRTQARCRGFLARVEYQKMVERRESIFCIQYNVRAFMNVKHWPWMKLYFKIKPLLKSAETEKEMANMKEEFEKTKEELAKSEAKRKELEEKMVTLTQEKNDLQLQVQSEADALADAEERCDQLIKTKIQLEAKIKEVTERAEDEEEINAELTAKKRKLEDECSELKKDIDDLELTLAKVEKEKHATENKVKNLTEEMAGLDETIAKLTKEKKALQEAHQQTLDDLQAEEDKVNTLTKAKTKLEQQVDDLEGSLEQEKKLRMDLERAKRKLEGDLKLAQESTMDIENDKQQLDEKLKKKEFEMSNLQSKIEDEQALAMQLQKKIKELQARIEELEEEIEAERASRAKAEKQRSDLSRELEEISERLEEAGGATSAQIEMNKKREAEFQKMRRDLEEATLQHEATAAALRKKHADSVAELGEQIDNLQRVKQKLEKEKSEMKMEIDDLASNMETVSKAKGNLEKMCRALEDQLSELKTKEDEQQRLINDLTTQRARLQTESGEFSRQLDEKDALVSQLSRGKQAFTQQIEELKRQLEEEIKAKSALAHALQSARHDCDLLREQYEEEQEGKAELQRAMSKANSEVAQWRTKYETDAIQRTEELEEAKKKLAQRLQDAEEHVEAVNAKCASLEKTKQRLQNEVEDLMIDVERTNAACAALDKKQRNFDKILSEWKQKYEETHAELEASQKESRSLSTELFKIKNAYEESLDQLETLKRENKNLQQEISDLTEQIAEGGKRIHELEKVKKQVEQEKSEIQAALEEAEASLEHEEGKILRIQLELNQVKSEIDRKIAEKDEEIDQLKRNHIRVVESMQSTLDAEIRSRNDAIRLKKKMEGDLNEMEIQLNHANRMAAEALKNYRNTQAILKDTQIHLDDALRGQEDLKEQLAMVERRANLLQAEIEELRATLEQTERSRKIAEQELLDASERVQLLHTQNTSLINTKKKLETDITQIQGEMEDIIQEARNAEEKAKKAITDAAMMAEELKKEQDTSAHLERMKKNLEQTVKDLQHRLDEAEQLALKGGKKQIQKLEARVRELEGEVESEQKRNVEAVKGLRKHERRVKELTYQTEEDRKNILRLQDLVDKLQAKVKSYKRQAEEAEEQSNVNLSKFRKLQHELEEAEERADIAESQVNKLRVKSREVHTKIISEE

SEQ ID NO: 136 - protein, 6909 aa, Bison bison
MADDEEYEEVVEYYTEETVYKEVPGETITGFYETTTTRTSDYEQSETSRPALAQPVPEKPVERKRVIRKKVDPSKFMTPYIAHSQKMQNLFSTNKYKENYEKAKGKPYAITTDTPELRRIKKVQDQLSEVKYRVDGDVAKTICHVDEKAKDIEHAKKVSQQVSKVLYKQNWEDTKDKYLLPPDAPELVQAIKNTAMFSKKLYTEDWEADKTMFYPYNDSPELRRVAQAQKALSDIAYKKGLAEQQTQFTSLPDPPDIEFAKKVTNQVSKQKYKEDYENKVKGKWSETPCFEIATARMNSNNISTKKYQEDFEHMKDQIYFMQTETPEYKVNKQAGVAASKVKYKQDYEKNKGKADYNVLPASENPLLRQLKAAGDALSDKLYKENYEKTKAKSINYCETPKFKLDTVLHNFSSDTKYKDSYLKDILGHYVGSYEDPYHTHCMRVSAQNSDKNYKAEYEEDRGKGFFPQTITQEYEAIKKLDQCKDHTYKVHPDKTKFTQVTDSPVLMQAQVNSKQLSDLNYKAKHENEKFKCHIPPDTPAFIQHKLNAYNLSDNIYKHDWEKTKAKKFDIKVDAIPLLAAKANTKIASDVMYKKDYEKSKGKMIGALSINDDPKMLHSLKTAKNQSDRLYKENYEKTKAKSMNYCETPKYQLDTLLKNFSEAKYKDSYVQNVLGHYIGSFEDPYQVHCLKISAQNSDKNYKAEYEEDKGKCYFPQTITQEYEAIKKLDQCKDHTYKVHPDKTKFTAVTDTPVLLQAQLNTKQLSDLNYKAKHEGEKFKCHIPADAPQFIQHRVNAYNLSDNIYKHDWEKTKAKKFDIKVDAIPLLAAKANTKIASDVMYKKDYEKSKGKMIGALSINDDPKMLHSLKTAKNQSDHEYRKDYEKSKTIYTAPLDMLPLTHAKKSQAIASDVDYKHLLHNYSYPPDSVNVDLAKKAYALQSDVEYKADYNSWMKGCGWMPFGSLEMEKAKRASDILNEKKYRQHPDTLKFTSIEDAPIIVQSKINQAQRSDVAYKAKGEEVIHKYSLPADLPQFIQAKVNAYNISENLYKADLKDLSKKGYDLRIDAIPIKAAKAARQAASDVQYKKDYEKAKGKMVGFQSLQDDPKLVHYMNVAKIQSDREYKKAYEKTKTRHNTPHDMVNIVAAKKAQDVASNVNYKHSLHHYTYLPDAMDLELSKNMMHIRSDNVYKEDYNNWMKGIGWIPIGSLEVEKVKKAGDALNEKKYRQHPDTLKFTSIVDSPVMVQAKQNTQQVSDILYKAKGEDVKHKYTMSPDLPQFLQAKCNAYNLSDVCYKRDWHDLIAKGTNVLGDAIPITAAKASRNIASDYKYKEAYEKAKGKQVGFRSLQDDPKLVHYMNVAKLQSDREYKKNYENTKTSYHTPGDMVSITAAKMAQDVATNVNYKQPIHHYTYLPDALSLEHIRNVNQIQSDNVYKDEYNHFFKGMGWIPIGSLEVEKVKKAGDALNEKKYRQHPDTIKFTSVPDSMGMVLAQHNTKQLSDLNYKVEGEKVKHKYTMDPDVPQFIQAKVNAYNMSDSHYKADWKKTLAKGYDLRPDAIPIVAAKSSRNIASDFKYKEAYEKTKGKQIGFRSLQDDPKLVHFMNVAKMQSDREYKKGYEARKTKYHTPLDMLSVTAAKKSQEVATNTNYKQPFHHYTLLPDALNVEHSRNAMQIQSDNLYKSDFTNWMKGIGWLPLESLEVEKAKKAGEILSEKKYRQHPEKLKFTYAMDTMEQALNKSNKLIMDKRLYTEKWNKDKTTIHVMPDTPDILLSRVNQITMSDKLYKAGWEEEKKKGYDLRPDAISIKAARASRDIASDYKYKQAYEQAKGRQIGFRSLEDDPKLVHFMQVAKMQSDREYKKAYEKSKTSFHTPVDMLSVVAAKKSQEVATNANYRNVIHTYNMLPDAMSLELAKNMMQIQSDNQYKADYADFMKGIGWLPLGSLEAEKNKKAMEIISEKKYRQHPDTLKYSTLMDSMNMVLAKNNAKIMNEHLYKQAWEADKTKVHIMPDIPQIILAKANAINISDKLYKLSLEEAKKKGYDLRTDAIPIKAAKASRDIASDYKYKHSYEKERGKMVGFRSLEDDPKLVHSMQVAKMQSDREYKKNYEKTKTSYHTPADMLSVTAAKDAQANITNTNYKHLIHKYILLPDAMNIQLSKNMNRIQSDNEYKQDYNEWYKGLGWSPAGSLEVEKAKKATEYASDQKYRQHPSNFQFTKLNDSMDMVLAKQNAHTMNKYLYTIDWNKDKTKIHVMPDTPDILQAKQNQTMYSQKLYKLGWEEALKKGYDLPVDAISVQAAKASRDIVSDFKYKQGYRKQIGHHIGFRSLQDDPKLVLSMNVAKMQSEREYKKDFEKWKTKFSSPVDMLGVVLAKKCQALVSDVDYKNYLHGWTCLPDQNDVIHAKKAYDLQSENLYKSDLEWLKGIGWSPLGSLEAEKNKRASEIISEKKYRQPPDRNKFTSIPDAMDIVLAKTNAKNRSDILYREAWDKDKTQIHIMPDTPDIILAKANLINTSDKFYRMGYEELRKKGYDLPVDAIPIKAAKASREIASEYKYKEGFRMQLGHHIGARNIKDDPKMMWSMHVAKIQSDREYKKDFEKWKTKFSSPVDMLGVVLAKKCQTLVSDIDYKNYLHQWTCLPDQSDVIHARRAYDLQSDNLYKSDLQWLRGIGWLPSGSLEDEKNKRASQILSDHVYRQHPDQFKFSSLMDSIPMVLAKNNAITMNHRLYTEAWDKDKTTVHIMPDTPEVLLAKQNQINYSEKLYKLGLDEAKRKGYDMRIDAIPIRAAKASRDIASEFKYKEGYRRQLGHHIGARAIHDDPKMMWSMHVAKIQSDREYKKDFEKWKTKFSSPVDMLGVVLAKKCQTLVSDIDYKNYLHQWTCLPDQSDVIHARRAYDLQSDNVYKSDLQWLRGIGWVPIGSLDVEKSKRASEILSDKLYRQPPDKFKFTSVTDSLEQVLAKNNAINMNKRLYTEAWDKDKTQVHIMPDTPEITLARTNKVNYSENLYKLAHEEAKKKGYDLRSDAIPIVAAKASRDIISDYKYKDGYRKQLGHHIGARDIKDDPKMMWSMHVAKIQSDREYKKDFEKWKTKFSSPVDMLGVVLAKKCQTLVSDIDYKNYLHQWTCLPDQSDVIHARRAYDLQSDNVYKSDLQWLRGIGWVPIESVDVVKCKRAAEILSDNLYRQPPDTLKFTSVPDSLEQVLAKNNAINMNKRLYTEAWDKDKTQIHIMPDTPEIMLARQNKLNYSETLYKLANEEAKKKGYDLRSDAIPIVAAKASRDIISDYKYKDGYRKQLGHHIGARDIKDDPKMMWSMHVAKIQSDREYKKDFEKWKTKFSSPVDMLGVVLAKKCQTLVSDIDYRNYLHQWTCLPDQSDVIHARQAYDLQSDNIYKSDLQWLRGIGWVPIGSVDVVKCKRAAEILSDNLYRQRPDTLKFTSVPDSLEQVLAKNNAINMNKRLYTEAWDKDKTQIHMMPDTPEITLARQNKINYSENLYRQAMEEAKKEGYDLRSDAIPIVAAKASREIASDYKYKEAYRKQLGHHIGARAIHDDPKMMWSVHVAKMQSDREYKKDFEKYKTRFSSPVDMLGIVLAKKCQTLVSDVDYKHPLHEWTCLPDQNDIIHARKAYDLQSDNLYKSDLEWLKGIGWVPIGSVEVLKAKRAGEILSDNIYRQRPDTLKFTSVTDSPEQVLAKNNAINMNKRLYTEAWDNDKKTIHVMPDTPEIMLAKLNRINYSDKLYKLALEESRKEGYDLRLDAIPIQAAKASREIASDYKYKEGYRKQLGHHIGARNIKDDPKMMWSIHAGKIQSDLEYKKDFEKWKTKFSSPVDMMGLVQAKKCQILVSDIDYKNLLHEWTCLPDQNDIIQARKAYDLQSDAIYKADLEWLRGIGWVPIDSVGVEHAKRAGEILSERKYRQPAVQLKFTSITDTPEIVLAKNNALNVSKHLYTEAWDADKTSIHVMPDTPEILLAKSNSANISHKLYTKGWDESKMKDYDLRADAISIKSAKASRDIASDYKYKEAYEKHKGHHIGARSVEDDPRIMCAMNAGRIQSEREYKKEFQKWKTKFSSPVDMLGILLAKKCQTLVSDIDYRNYLHHWTCLPDQNDIIQARKAYDLQSDAIYKADLEWLRGIGWMPEGSPEVLRVKNAQHIFRDSVYRTPVVKLKYTSIVDTPEVVLAKSNAENISIPKYREVWDKDKTSIHIMPDTPEINLARTNALNVSNKLYREGWDEMKMSCDVRLDAIPIQAAKASREIASDYKYKLDHEKQKGHYVGTRTARDDNKIRWALIAGKIQNEREYRLHWAKWKSKFQSPPDMLSIEHSKNSQTLVSDIDYRHYLHQWTCMPDQNDVIQARKAYDLQSDAIYKADLEWLRGIGWMPADSVSVNHAKHMADIFNEKKYRTKIETLSFTPVDDRVDYVTAKHSGEILNDIKYRKDWNDTKSKYTLTETPQLHTAQEAARILDQYLYKESWEKQKATGYILPPDAVPFVHAHHSGDVQSELKYKAEHVKQKGHYVGVPTMRDDPKLVWFEHAGQIQNDRLYKENYHKTKAKIHIPPDMVSVLAAKEGQALASDIDYRNYLHQWICHPDQNDVIQARKAYDLQSDNIYKADLEWLRGIGWIPLDSVDHVRVTRNQEMMNQIKYKKDALANYPNFTSVVDPPEIVLAKINSVNQSDVKYKETFNKLIKGKYIFSPDTPYITHSKDMEKLYSTILYKRAWDGTKAYGYTLDERYIPIVGAKHADFVNSELKYKETYEKLKGHYLAGKEISEFPNVVHCLDFQKMRSLLNYRRHYEDTKANVHIPQDMMNHVLAKRCQYILSDLEYRHYFHQWTSLPEEPNVVRVRHAQEILSDNVYKDDLNWLKGIGCYVWDTPQILHAKKSYDLQSQILYTAAGKENLKNYNLVTDTPLYVTALQSGINASEVKYKENYHQIKDKYTTVLETVDYDRIKNLKDLFSSNLYKEAWDKVKATSYILPPNTVSLTHAKNQKYMASHIKYREEYEKFKALYTLPRSVEDDPNTARCLRVGKFNIDRLYRSVYEKNKMKIHIVPDMVEMVTAKDSQKKVSEIDYRLHLHEWICHPDLQVNSHVRKVTDQISDIVYKDDLTWLKGIGCYVWDTPEILHAKHAYDLRNDIKYKAHVLKTRNNYKLVTDTPVYVQAVKSGKQLSDAVYHYDYVHSIRGRVAPTTKTVDLDRALHAYKLQSENLYRKAGLHALPTGYRLPVDTPHFKHTKDTRYMSSYFKYKEVYEHMKAYGYTLGPNDVPFVNVRRVNNITSERLYRQLYHKLKDKIHTTPDTPEIRQVKKTQEAVSELIYKSDFFKMQGHMISLPYTPQVLHCRYVGDITSDIKYKEDLQVLRGMGCFLYDTPDMVRSRHLRKLWSHYLYTDKARKMRDKYKVVLDTPEYRKVQELKTHLSELVYRAAGRKQKSIFTSVPDTPDLTRAKRGQKLQSQYLYVELATKERPHHHAGNQTTALKHARHVKDMVSENKYKIQYEKMKDKYTPVPDTPILIRAKRAYWNASDLRYKETFQKTKGKYHTVKDALDIVYHRTVTDHVSKIKYKENYMSQLGIWRSIPDRPEHFHHRAVTDAVSDVKYKQDLTWLKGIGCYAYDTPDFTLAEKNKTLYSKYKYKEVFERTKSNFKYVADCPINRHFKFATQLMNEKKYRADYEQRKDKYHLVVDEPRHLLAKIAGDQISQIKYRKNYEETKDKFTSIVDTPEHLRTTKVNKQISDILYKLEYNKAKPRGYTTIHDTPMLLHVRKVKDEVSDLKYKEVYQRTKSNCTIEPDAVHIKAAKDAYKVNTNLDYKKKYEATKAYWKWTPDRPDFIQAAKSTLQQSDFEYKLDREYLKGCKLSVTDDKDMVLALKNSIIESDLKYKEKHVKERGSCHAVPDTPQILLAKTVSSLVSENKYKSYVKKHLAQGSYTTLPETRDTIHVKEVTKNVSDTNYKKKFVKEKGKSNYSIMLEPPDVKHAMDVAKKQSNVAYKKDAKENLHYTTVADRPDIKKATQAAKQASEVEYRAKHRKEGSHGLSMLGRPDIEMAKKAAKLSSQVQYRENFNKEKGKTPKYNPKDSQLYKVMKDANTLASEVKYKADLKKLHKPVTDMKESLIMNHVLNTSHLASSYQYKKNYEKSKGHYHTIPDNLEQLHLKEATELQSIVKYKEKYEKERGKPMLDFETPTYITAKESQQMQSGKEYRKDYEESIKGRNLTGLEVTPALLHVKYATKIASEKEYRKDLEESIRGKGLSEMEDTPDMLRAKNATQILNEKEYKRDLELEVKGRGLNAMANETPDFLRARNATDIASQIKYKQSAEMEKANFTSVVDTPEIIHAQQVKNLSSQKKYKEDAEKCMSYYETVLDTPEMQRVRENQKNFSLLQYQYDLKNSKGKITVVQDTPEILRVKENQKNFSSVLYKEDVSPGTAIGKTPEMMRVKQTQDHISSVKYKEVIGQGTPIPDLPEVKRVKQTQKHISSVMYKENLGTGIPTPVTPEIERVKRNQENFSSVLYKENLGKGTPTPITPEMERVKRNQENFSSVLYKENMGKGTPLPVTPEMERIKHNQENISSVLYKENVGKGTPTPVTPEMQRVKRNQENISSVLYKENLGKATPTPFTPEMERVKRNQENFSSVLYKENMRKATPTPVTPEMERAKRNQENISSVLYSDSFRKQIQGKAAYVLDTPEMRRVRETQRHISTVKYHEDFEKHKGCFTPVVTDPITERVKKNTQDFSDICYRGIQRKVVEMEQKRNDQDQETITGLRVWRTNPGSVFDYDPAEDNIQSRSLHMINAQAQRRSREQSRSASGLSISGGEEKSEHSEAAHLSTYSDGGVFFSAASTGYKHARTTELPQQRSSSVATQQTTVSSIPSHPSTAGKIFRAMYDYMAADADEVSFKDGDAIVNVQAIDEGWMYGTVQRTGRTGMLPANYVEAI

SEQ ID NO: 137 - protein, 1938 aa, Bison bison
MSSDQEMAIFGEAAPYLRKSEKERIEAQNKPFDAKTSVFVVDPKESFVKATVQSREGGKVTAKTEAGATVTVKEDQIFPMNPPKYDKIEDMAMMTHLHEPAVLYNLKERYAAWMIYTYSGLFCVTVNPYKWLPVYNAEVVTAYRGKKRQEAPPHIFSISDNAYQFMLTDRENQSILITGESGAGKTVNTKRVIQYFATIAVTGEKKKEEPTSGKMQGTLEDQIISANPLLEAFGNAKTVRNDNSSRFGKFIRIHFGTTGKLASADIETYLLEKSRVTFQLKAERSYHIFYQIMSNKKPELIEMLLITTNPYDYAFVSQGEITVPSIDDQEELIATDSAIDILGFTSDERVSIYKLTGAVMHYGNLKFKQKQREEQAEPDGTEVADKAAYLQSLNSADLLKALCYPRVKVGNEFVTKGQTVQQVYNAVGALAKAVYEKMFLWMVTRINQQLDTKQPRQYFIGVLDIAGFEIFDFNSLEQLCINFTNEKLQQFFNHHMFVLEQEEYKKEGIEWEFIDFGMDLAACIELIEKPMGIFSILEEECMFPKATDTSFKNKLYEQHLGKSNNFQKPKPAKGKAEAHFSLVHYAGTVDYNIAGWLDKNKDPLNETVVGLYQKSAMKTLAFLFAGGPSAEEGGGGKKGGKKKGSSFQTVSALFRENLNKLMTNLRSTHPHFVRCIIPNETKTPGAMEHELVLHQLRCNGVLEGIRICRKGFPSRILYADFKQRYKVLNASAIPEGQFIDSKKASEKLLGSIDIDHTQYKFGHTKVFFKAGLLGILEEMRDEKLAQLITRTQAICRGFLMRVEFRKMMERRESIFCIQYNVRAFMNVKHWPWMKLYFKIKPLLKSAETEKEMANMKEEFEKTKEELAKSEAKRKELEEKMVTLMQEKNDLQLQVQSEADGLADAEERCDQLIKTKIQLEAKIKELTERAEDEEEINAELTAKKRKLEDECSELKKDIDDLELTLAKVEKEKHATENKVKNLTEEMAGLDENIAKLTKEKKALQEAHQQTLDDLQAEEDKVNTLTKSKTKLEQQVDDLEGSLEQEKKLRMDLERAKRKLEGDLKLAQESTMDIENDKQQLDEKLKKKEFEMSNLQSKIEDEQALAMQLQKKIKELQARIEELEEEIEAERASRAKAEKQRSDLSRELEEISERLEEAGGATSAQIEMNKKREAEFQKMRRDLEEATLQHEATAAALRKKHADSVAELGEQIDNLQRVKQKLEKEKSEMKMEIDDLASNMETVSKAKGNLEKMCRTLEDQLSEVKSKEEEHQRLINELSAQKARLHAESGEFSRQLEEKDALVSQLSRGKQAFTQQIEELKRQLEEETKAKSALAHALQSARHDCDLLREQYEEEQEGKAELQRALSKANSEVAQWRTKYETDAIQRTEELEEAKKKLAQRLQDAEEHVEAVNAKCASLEKTKQRLQNEVEDLMLDVERSNAACAALDKKQRNFDKVLSEWKQKYEETQAELEASQKESRSLSTELFKVKNACEESLDQLETLKRENKNLQQEISDLTEQIAEGGKHIHELEKVKKQIEQEKGELQAALEEAEASLEHEEGKILRIQLELNQVKSEIDRKIAEKDEEIDQLKRNHLRVMESTQSTLDAEIRSRNDALRIKKKMEGDLNEMEIQLNHANRQATEAIKNLRNTQGVLKDTQLHLDDAIRGQDDLKEQLAMVERRANLMQAEIEELRASLEQTERSRRVAEQELLDASERVQLLHTQNTSLINTKKKLETDITQIQGEMEDILQEARNAEEKAKKAITDAAMMAEELKKEQDTSAHLERMKKNMEQTVKDLQHRLDEAEQLALKGGKKQIQKLEARVRELENEVENEQKRNAEAVKGLRKHERRVKELTYQTEEYRKNVLRLQDLVDKLQSKVKAYKRQAEEAEEQSNVNLAKFRKLQHELEEAEERADIAESQVNKLRVKSREVHTKVISEE

SEQ ID NO: 138 - protein, 607 aa, Bison bison
MKWVTFISLLLLFSSAYSRGVFRRDTHKSEIAHRFKDLGEEHFKGLVLIAFSQYLQQCPFDEHVKLVNELTEFAKTCVADESHAGCEKSLHTLFGDELCKVASLRETYGNMADCCEKQEPERNECFLSHKDDSPDLPKLKPDPNTLCDEFKADEKKFWGKYLYEIARRHPYFYAPELLYYANKYNGVFQECCQAEDKGACLLPKIETMREKVLTSSARQRLRCASIQKFGERALKAWSVARLSQKFPKAEFVEVTKLVTDLTKVHKECCHGDLLECADDRADLAKYICDNQDTISSKLKECCDKPLLEKSHCIAEVEKDAVPENLPPLTADFAEDKDVCKNYQEAKDAFLGSFLYEYSRRHPEYAVSVLLRLAKEYEATLEECCAKDDPHACYSTVFDKLKHLVDEPQNLIKQNCDQFEKLGEYGFQNALIVRYTRKVPQVSTPTLVEVSRSLGKVGTRCCTKPESERMPCTEDYLSLILNRLCVLHEKTPVSEKVTKCCTESLVNRRPCFSALTPDETYVPKAFDEKLFTFHADICTLPDTEKQIKKQTALVELLKHKPKATEEQLKTVMENFVAFVDKCCAADDKEACFAVEGPKLVVSTQTALA

SEQ ID NO: 139 - protein, 667 aa, Cervus elaphus
MLDAEDIVNTPKPDEKAIMTYVSCFYHAFAGAEQAETAANRICKVLAVNQENEKLMEEYEKLASELLEWIRRTVPWLENRVGEPSMSAMQRKLEDFRDYRRLHKPPRVQEKCQLEINFNTLQTKLRLSHRPAFMPSEGKLVSDIAKAWRGLEQVEKGYEDWLLSEIRRLQRLQHLAEKFQQKASLHEAWTRGKEEMLSQRDYETASLQEVRALLRRHEAFESDLAAHQDRVEHIAALAQELNELDYHEAASVNSRCQAICDQWDNLGTLTQKRRDALERMEKLLETIDQLQLEFARRAAPFNNWLDGAVEDLQDVWLVHSVEETQSLVTAHDQFKATLPEADRERGAILGIQGEIQKICQTYGLRPSSTNPYIALTPQDINTKWDTVRKLVPSRDQMLQEELTRQQVNERLRRQFAAQANAIGPWIQGKVEEVGRLAAGMAGSLEEQMAGLRQQEQNIINYKSNIDRLEGDHQLLQESLVFDNKHTVYSMEHIRVGWEQLLTSIARTINEVENQVLTRDAKGLSQEQLNEFRASFNHFDRKRNGMMEPDDFRACLISMGYDLGEVEFARIMTMVDPNAAGVVTFQAFIDFMTRETAETDTAEQVVASFKILAGDKNYITAEELRRELPAEQAEYCIRRMAPYKGAGAPAGALDYVAFSSALYGESDL

SEQ ID NO: 140 - protein, 792 aa, Cervus elaphus
MSRPLTDQEKRKQISVRGLAGVENVTELKKNFNRHLHFTLVKDRNVATPRDYYFALAYTVRDHLVGRWIRTQQHYYEKDPKRIYYLSLEFYMGRTLQNTMVNLALENACDEATYQLGLDMEELEEIEEDAGLGNGGLGRLAACFLDSMATLGLAAYGYGIRYEFGIFNQKISGGWQMEEADDWLRYGNPWEKARPEFTLPVHFYGRVEHTSQGAKWVDTQVVLAMPYDTPVPGYRNNVVNTMRLWSAKAPNDFNLKDFNVGGYIQAVLDRNLAENISRVLYPNDNFFEGKELRLKQEYFVVAATLQDIIRRFKSSKFGCLDPVAIQLNDTHPSLAIPELMRILVDQERLEWEKAWEVTVKTCAYTNHTVLPEALERWPVHLIETLLPRHLQIIYEINQRFLNRVAAAFPGDVDRLRRMSLVEEGAVKRINMAHLCIAGSHAVNGVARIHSEILKKTMTSKSWSLINSRIRPMGSPLGAGWLCRIGEEYIADLDQLRKLLSYVDDESFIRDVAKVKQENKLKFSAYLEKEYKVHINPNSLFDIQVKRIHEYKRQLLNCLHAAPGYHMAKMIIKLITAIGDVVNHDPVVGDRLRVIFLENYRVSLAEKVIPAADLSEQISTAGTEASGTGNMKFMLNGALTIGTMDGANVEMAEEAGEENFFIFGMRVEDVERLDQRGYNAQEYYDRIPELRHVIDQLSSGFFSPKQPDLFKDIVNMLMHHDRFKVFADYEEYVKCQERVSALYKNPREWTRMVIRNIATSGKFSSDRTIAQYAREIWGVEPNRQRMPAPDEKI

SEQ ID NO: 141 - protein, 412 aa, Cervus elaphus
MAQRKPEGSGFHMTCLSMALAYSFRPDANIQPHPQLGNTQKQTELGKELTGTMPHQYPALTPEQKKELCDIAHRIVAPGKGILAADESTGSIAKRLQSIGTENTEENRRFYRQLLLTADDRVNPCIGGVILFHETLYQKADDGRPFPQVIKAKGGVVDKGVVPLAGTNGETTTQGLDGLSERCAQYKKDGADFAKWRCVLKIGEHTPSSLAIMENANVLARYASICQQNGIVPIVEPEILPDGDHDLKRCQYVTEKVLAAVYKALSDHHIYLEGTLLKPNMVTPGHACTQKYSHEEIAMATVTALRRTVPPAVPGITFLSGGQSEEEASINLNAINKCPLLKPWALTFSYGRALQASALKAWGGKKENLKAAQEEYVKRALANSLACQGKYTPSGKAGAAASESLFISNHAY

SEQ ID NO: 142 - protein, 167 aa, Cervus elaphus
PAPAPPKEEKIDLSAIKIEFSKQQQDEFKEAFLLFDRTGECKITLNQVGDVLRALGTNPTNAEVKKVLGNPSNEEMNAKKIEFEQFLPMLQAISNNKDQGTYEDFVEGLRVFDKEGNGTVMGAELRHVLATLGEKMKEEEVEALMAGQEDSNGCINYEAFVKHIMSN

SEQ ID NO: 143 - protein, 206 aa, Cervus elaphus
MFVMGVNHEKYNNTLKIVSNASCTTNCLAPLAKVIHDHFGIVEGLMTTVHAITATQKTVDGPSGKLWRDGRGAAQNIIPASTGAAKAVGKVIPELNGKLTGMAFRVPTPNVSVVDLTCRLEKPAKYDEIKKVVKQASEGPLKGILGYTEDQVVSCDFNSDTHSSTFDAGAGIALNDHFVKLISWYDNEFGYSNRVVDLMVHMASKE

SEQ ID NO: 144 - protein, 428 aa, Cervus elaphus
MRTLRAMAMQKIFAREILDSRGNPTVEVDLHTAKGRFRAAVPSGASTGIYEALELRDGDKSRYLGKGVLKAVEHINKTLGPALLEKASLGRLYLLPPAKFGANAILGVSLAVCKAGAAEKGVPLYRHIADLAGNPELILPVPAFNVINGGSHAGNKLAMQEFMILPVGASSFKEAMRIGAEVYHHLKGVIKAKYGKDATNVGDEGGFAPNILENNEALELLKTAIQAAGYPDKVVIGMDVAASEFYRNGKYDLDFKSPDDPARHISGEKLGELYKNFIKNYPVVSIEDPFDQDDWATWTSFLSGVNIQIVGDDLTVTNPKRIAQAVEKKACNCLLLKVNQIGSVTESIQACKLAQSNGWGVMVSHRSGETEDTFIADLVVGLCTGQIKTGAPCRSERLAKYNQLMRIEEALGDKAVFAGRKFRNPKAK

SEQ ID NO: 145 - protein, 181 aa, Cervus elaphus
MFDQTQIQEFKEAPKKAKRRAAAEGGSSSVFSMFDQTQIQEFKEAFTVIDQNRDGIIDKEDLRDTFAAMGRLNVKNEELDAMMKEASGPINFTVFLNMFGEKLKGADPEDVITGAFKVLDPEGKGTIKKQFLEELLTTQCDRFSQEEMRNMWAAFPPDVGGNVDYKNICYVITHGDAKDQE

SEQ ID NO: 146 - protein, 340 aa, Cervus elaphus
MCEEEDSTALVCDNGSGLCKAGFAGDDAPRAVFPSIVGRPRHQGVMVGMGQKDSYVGDEAQSKRGILTLKYPIEHGIITNWDDMEKIMFETFNVPAMYVAIQAVLSLYASGRTTGIVLDSGDGVTHNVPIYEGYALPHAIMRLDLAGRDLTDYLMKILTERGYSFVTTAEREIVRDIKEKLCYVALDFENEMATAASSSSLEKSYELPDGQVITIGNERFRCPETLFQPSFIGMESAGIHETTYNSIMKCDIDIRKDLYANNVLSGGTTMYPGIADRMQKEITALAPSTMKIKIIAPPERKYSVWIGGSILASLSTFQQMWISKQEYDEAGPSIVHRKCF

SEQ ID NO: 147 - protein, 605 aa, Cervus elaphus
MKWVTFISLLLLFSSAYSRGVFRRDTHKSEIAHRFKDLGEDNFQGLVLIAFSQYLQQCPFDEHVKLVKELTDFAKECVADESHAGCEKSLHTLFGDELCKVASLRETYGDMADCCEKQEPERNECFLKHKDDNPDLPKLKPDPDTLCAEFKADEKKFWGKYLYEVARRHPYFYAPELLYYANKYNGVFQECCQADDKAACLLPKIDTMREKVLTSSARQRLKCASIQKFGERALKAWTVALLSQKFPKADFAEVTKIVTDLTKVHKECCHGDLLECADDRADLAKYICDNQDTLSSKLKECCDKPVLEKSHCIAEIEKDAVPENLPPLTADFAEDKEVCKNYQEAKDVFLGTFLYEYARRHPEYAVSVLLRVAKEYEATLEDCCAKDDPHACYATVFDKLQHLVDEPQNLIKQNCDLFEKVGEYGFQNALIVRYTKKAPQVSTPTLVEVSRNLGRVGTKCCTKPESERMSCTEDYLSLVLNRLCVLHEKTPVSEKVTKCCTESLVNRRPCFSALTLDETYVPKAFDEKLFTFHADICTVPDAEKQIKKQTALVELLKHKPKATDEQLKTVMENFVAFVDKCCAAVDKEACFAVEPTLRVRGKKEK

SEQ ID NO: 148 - protein, 381 aa, Gallus gallus
MPFSSTHNKHKLKFSAEEEFPDLSKHNNHMAKVLTPELYKRLRDKETPSGFTLDDVIQTGVDNPGHPFIMTVGCVAGDEESYEVFKDLFDPVIQDRHGGYKPTDKHRTDLNHENLKGGDDLDPKYVLSSRVRTGRSIKGYSLPPHCSRGERRAVEKLSVEALNSLEGEFKGRYYPLKAMTEQEQQQLIDDHFLFDKPVSPLLLASGMARDWPDARGIWHNDNKTFLVWVNEEDHLRVISMEKGGNMKEVFRRFCVGLKKIEEIFKKAGHPFMWTEHLGYILTCPSNLGTGLRGGVHVKLPKLSQHPKFEEILHRLRLQKRGTGGVDTAAVGAVFDISNADRLGFSEVEQVQMVVDGVKLMVEMEKKLEQNQPIDDMIPAQK

SEQ ID NO: 149 - protein, 897 aa, Gallus gallus
MNSMNQIETNMQYTYNYEEDEYMTQEEEWDRDLLLDPAWEKQQRKTFTAWCNSHLRKAGTQIENIEEDFRNGLKLMLLLEVISGERLPKPDRGKMRFHKIANVNKALDYIASKGVKLVSIGAEEIVDGNVKMTLGMIWTIILRFAIQDISVEETSAKEGLLLWCQRKTAPYRNVNIQNFHLSWKDGLGLCALIHRHRPDLIDYSKLNKDDPIGNINLAMEIAEKHLDIPKMLDAEDIVNTPKPDERAIMTYVSCFYHAFAGAEQAETAANRICKVLAVNQENERLMEEYERLASELLEWIRRTIPWLENRTPEKTMQAMQKKLEDFRDYRRKHKPPKVQEKCQLEINFNTLQTKLRISNRPAFMPSEGKMVSDIAGAWQRLEQAEKGYEEWLLNEIRRLERLEHLAEKFRQKASTHEQWAYGKEQILLQKDYESASLTEVRAMLRKHEAFESDLAAHQDRVEQIAAIAQELNELDYHDAASVNDRCQKICDQWDSLGTLTQKRREALERTEKLLETIDQLHLEFAKRAAPFNNWMEGAMEDLQDMFIVHSIEEIQSLISAHDQFKATLPEADGERQAILSIQNEVEKVIQSYSMRISASNPYSTVTVEEIRTKWEKVKQLVPQRDQSLQEELARQHANERLRRQFAAQANVIGPWIQTKMEEIARSSIEMTGPLEDQMNQLKQYEQNIINYKHNIDKLEGDHQLIQEALVFDNKHTNYTMEHIRVGWELLLTTIARTINEVETQILTRDAKGITQEQMNDFRASFNHFDRRKNGLMDHDDFRACLISMGYDLGEAEFARIMSLVDPNGQGTVTFQSFIDFMTRETADTDTAEQVIASFRILASDKPYILADELRRELPPEQAQYCIKRMPQYTGPGSVPGALDYTSFSSALYGESDL

SEQ ID NO: 150 - protein, 843 aa, Gallus gallus
MAAPLSDGERRKQISVRGIAGLGDVAEVRKSFNRHLHFTLVKDRNVATPRDYFFALAHTVRDHLVGRWIRTQQHYYERDPKRIYYLSLEFYMGRTLQNTMVNLGLQNACDEAIYQLGLDLEELEEIEEDAGLGNGGLGRLAACFLDSMATLGLAAYGYGIRYEFGIFNQKIIDGWQVEEADDWLRYGNPWEKARPEYMLPVHFYGRVEHSPEGVKWVDTQVVLAMPYDTPVPGYKNNTVNTMRLWSAKAPNDFNLQEFNVGDYIEAVLDRNLAENISRVLYPNDNFFEGKELRLKQEYFVVAATLQDIIRRFKSSKFGCQDPVRTSFETFPDKVAIQLNDTHPALSIPELMRILVDVEKVDWDKAWEITKQTCAYTNHTVLPEALERWPVSMFEKLLPRHLEIIYALNQMHLDRVAALYPGDVDRLRRMSVIEEGDCKRINMAHLCVIGSHAVNGVARIHSDIVKNTVFKDFYELEPEKFQNKTNGITPRRWLLLCNPGLADVIAEKIGEDFVTDLSQLKKLLDFINNETFIRDVAKVKQENKLKFSAYLEERYKVKINPSSMFDIQVKRIHEYKRQLLNCLHAITLYNRIRSDPSKSFVPRTIMIGGKAAPGYHMAKMIIKLITSIGEVVNNDPYVGDKLKVIFLENYRVSLAEKVIPAADLSQQISTAGTEASGTGNMKFMLNGALTIGTMDGANVEMAEEAGEENLFIFGMRVEEVEALDRRGYNAREYYDRIPELRQAIDQISSGFFSPRDPGCFRDVVNMLMHHDRFKVFADYEAYIKCQGQVDQLFMDPREWTKKVIRNIACSGKFSSDRTITEYAREIWGVEPSATKIPPPNLPRD

SEQ ID NO: 151 - protein, 364 aa, Gallus gallus
MTHQYPALTAEQKKELSDIALRIVAPGKGILAADESVGSMAKRLNQIGVENTEENRRLYRQILFSADSRVKKCIGGVIFFHETMYQKADDGTPFVQMIKDKGIVVGIKVDKGVVPLAGTDGETTTQGLDGLSERCAQYKKDGADFAKWRCVLKISDNTPSALAIMENANVLARYASICQQNGIVPIVEPEILPDGDHDLKRCQYVTEKVLAAVYKALSDHHVYLEGTLLKPNMVTPGHSCPTKYSPEEIAMATVTALRRTVPPAVPGVTFLSGGQSEEEASINLNAINTCPLVRPWALTFSYGRALQASALSAWRGQRDNANAATEEFVKRAEVNGLAALGKYEGSGDDSGAAGQSLYVANHAY

SEQ ID NO: 152 - protein, 192 aa, Gallus gallus
MAPKKDVKKPAAAAAPAPAPAPAPAPAPAKPKEPAIDLKSIKIEFSKEQQDDFKEAFLLFDRTGDAKITLSQVGDIVRALGQNPTNAEINKILGNPSKEEMNAKKITFEEFLPMLQAAANNKDQGTFEDFVEGLRVFDKEGNGTVMGAELRHVLATLGEKMTEEEVEELMKGQEDSNGCINYEAFVKHIMSV

SEQ ID NO: 153 - protein, 333 aa, Gallus gallus
MVKVGVNGFGRIGRLVTRAAVLSGKVQVVAINDPFIDLNYMVYMFKYDSTHGHFKGTVKAENGKLVINGHAITIFQERDPSNIKWADAGAEYVVESTGVFTTMEKAGAHLKGGAKRVIISAPSADAPMFVMGVNHEKYDKSLKIVSNASCTTNCLAPLAKVIHDNFGIVEGLMTTVHAITATQKTVDGPSGKLWRDGRGAAQNIIPASTGAAKAVGKVIPELNGKLTGMAFRVPTPNVSVVDLTCRLEKPAKYDDIKRVVKAAADGPLKGILGYTEDQVVSCDFNGDSHSSTFDAGAGIALNDHFVKLVSWYDNEFGYSNRVVDLMVHMASKE

SEQ ID NO: 154 - protein, 284 aa, Gallus gallus
MDAIKKKMQMLKLDKENALDRAEQAEADKKAAEERSKQLEDELVALQKKLKGTEDELDKYSESLKDAQEKLELADKKATDAESEVASLNRRIQLVEEELDRAQERLATALQKLEEAEKAADESERGMKVIENRAQKDEEKMEIQEIQLKEAKHIAEEADRKYEEVARKLVIIEGDLERAEERAELSESKCAELEEELKTVTNNLKSLEAQAEKYSQKEDKYEEEIKVLTDKLKEAETRAEFAERSVTKLEKSIDDLEDELYAQKLKYKAISEELDHALNDMTSI

SEQ ID NO: 155 - protein, 1132 aa, Gallus gallus
MPEPSKAAPKKEAKKKEEKKEEKKEAPPPQEHKDEAPDDVHPPETPDPEGLFLSKPQNVMVESGRDVTVSARVAGAALPCAPAVKWFKGKWAELGDKSARCRLRHSVDDDKVHTFELTITKVAMGDRGDYRCEVTAKEQKDSCSFSIDVEAPRSSEGNVLQAFKRTGEGKDDTAGELDFSGLLKKREVQVEEKKKKKDEDDQFPPEIWELLKGVTKKSEYERIAFQYGITDLRGMLKRLKKVHVEPKKSEAFIRKLDPAYQVDKGNKIKLVVELSDPDLPLKWYKNGQLLKPSTKYVFENVGLKRILTIHKCSLADDAAYECRVNDEKCFTEVFVKEPPVTVVRGLEDQQVVVGDRVVLEAEVSEEGAQVMWLKDGVDVTRDDAFKYRFKKDGKKHFLIINEAELSDSAHYKIMTNGGESEAELSVEEKQLEVLQDMADLTVKASEQAVFKCEVSDEKVTGRWFRNGVEVKPSKRIHISHNGRFHKLVIDDVRPEDEGDYTFIPDGYALSLSAKLNFLEIKVEYVPKQEPPKIHLDCSGKAAENTIVVVAGNKVRLDVPISGEPAPTVTWKRGDQLFTATEGRVHIDSQADLSSFVIESAERSDEGRYCITVTNPVGEDSATLHVRVVDVPDPPQSVRVTSVGEDWAVLSWEAPPFDGGMPITGYLMERKKKGSMRWMKLNFEVFPDTTYESTKMIEGVFYEMRVFAVNAIGVSQPSLNTQPFMPIAPTSEPTHVVLEDVTDTTATIKWRPPERIGAGGVDGYLVEWCREGSNEWVAANTELVERCGLTARGLPTGERLLFRVISVNMAGKSPPATMAQPVTIREIVERPKIRLPRHLRQTYIRRVGEQVNLVIPFQGKPRPQVTWSREGGALPAEVQTRTSDVDSVFFIRSAARPLSGNYEMRVRIDNMEDCATLRLRVVERPGPPQAVRVMEVWGSNALLQWEPPKDDGNAEISGYTVQKADTRTMEWFTVLEHSRPTRCTVSELVMGNEYRFRVYSENVCGTSQEPATSHNTARIAKEGLTLKMVPYKERDLRAAPQFLTPLVDRSVVAGYTVTLNCAVRGHPKPKVTWLKNSVEIGADPKFLSRHGLGVLSLLIRRPGPFDGGTYGCRAVNEMGEATTECRLDVRVPQ

SEQ ID NO: 156 - protein, 1450 aa, Gallus gallus
MSSVAVPFYQRRHKHFDQSYRNIQTRYVLEEYAARKAASRQAAHYESTGLGKTTCRLCARRARSLAHEAMQESRKRTHEQKSHASDEKRIKFASELSSLEREIHMARHHAREQLDRLAIQRMVEENMALERHVVEEKISRAPEILVRLRSHTVWEKMSVRLCFTVQGFPSPVVQWYKNEELITPASDPAKYSVENKYGVHVLHINRADFDDSATYSAVATNIHGQASTNCAVVVRRFRESEEPHPAGIMPFHLPLSYDVCFTHFDVQFLEKFGVTFATEGETLTLKCSVLVTPELKRLRPRAEWYRDDVLIKDSKWTKLYFGEGQAALSFTHLNKDDEGLYTLRMVTKGGVNECSAFLFVRDADALIAGAPGAPMDVKCHDANRDYVIVTWKPPNTTSQNPVIGYFVDKCEVGLENWVQCNDAPVKICKYPVTGLYEGRSYIFRVRAVNSAGISRPSRVSEPVAALDPVDLERTQTVHVDEGRKIVISKDDLEGDIQIPGPPTNVHASEISKTYVVLSWDPPVPRGREPLTYFIEKSMVGSGSWQRVNAQVAVKSPRYAVFDLAEGKPYVFRVLSANKHGISDPSEITEPIQPQDIVVVPSAPGRVVATRNTKTSVVVQWDKPKHEENLYGYYIDYSVVGSNQWEPANHKPINYNRFVVHGLETGEQYIFRVKAVNAVGFSENSQESEAIKVQAALTCPSYPHGITLLNCDGHSMTLGWKAPKYSGGSPILGYYIDKREANHKNWHEVNSSVISRTIYTVEDLTEDAFYEFKIAAANVVGIGHPSDPSEHFKCKAWTMPEPGPAYDLTVCEVRNTSLVLLWKAPVYEGKSPITGYLVDYKEVDTEDWITANEKPTSHRYFKVTDLHQGHTYVFKVRAVNDAGVGKSSEISEPVFVEASPGTKEIFSGVDEEGNIYLGFECKEATDASHFLWGKSYEEIEDSDKFKIETKGDHSKLYFKHPDKSDLGTYCISVSDTDGVSSSFVLDEEELERLMTLSNEIKNPTIPLKSELAYEVLDKGEVRFWIQAESLSPNSTYRFVINDKEVENGDRHKISCDHSNGIIEMVMDKFTIDNEGTYTVQIQDGKAKNQSSLVLIGDAFKAILAESELQRKEFLRKQGPHFSEFLYWEVTEECEVLLACKIANTKKETVFKWYRNGSGIDVDEAPDLQKGECHLTVPKLSRKDEGVYKATLSDDRGHDVSTLELSGKVYNDIILALSRVSGKTASPLKILCTEEGIRLQCFLKYYNEEMKVTWSHRESKISSGEKMKIGGGEDVAWLQITEPTEKDKGNYTFEIFSDKESFKRTLDLSGQAFDDALTEFQRLKAAAFAEKNRGKVIGGLPDVVTIMDGKTLNLTCTVFGNPDPEVVWFKNDKALELNEHYLVSLEQGKYASLTIKGVTSEDSGKYSIYVKNKYGGETVDVTVSVYRHGEKIPEVNQGQLAKPRLIPPSSST

SEQ ID NO: 157 - protein, 434 aa, Gallus gallus
MSIQKIHAREILDSRGEPTVEVDLHTAKGHFRAAVPSGASTGIHEALEPRDGDKKRFLGKGVLKAVEHINKTIGPALIEKKISVVEQEKIDKVMIEMDGTENKSKFGANAILGVSLAVCKAGAAEKGVPLYRHIADLAGNTELILPVPAFNVINGGSHAGNKLAMQEFMVLPVGAASFHDAMRVGAEVYHSLKGVIKAKYGKDATNVGGEGGFAPNILDNHEALELLKAAIAQAGYTDKVVIGMDVAASEFCRDGRYHLDFKSPPHTKRYITGEQLGEIYRGFIKDYPVVSIEDPFDQDDWEAWKRFVFHVDIQVVGDDLTVTNPKRIAHGAEQHACNCLLLKVNQIGSVTESIQACKLAQSHGWGVMVSHRSGETEDTFIADLVVGLCTGQIKTGAPCRSERLAKYNQLMRIEEALGDKAKFAGRKFRNPKAK

SEQ ID NO: 158 - protein, 168 aa, Gallus gallus
MAPKKAKRRAAEGSSNVFSMFDQTQIQEFKEAFTVIDQNRDGIIDKDDLRETFAAMGRLNVKNEELDAMIKEASGPINFTVFLTMFGEKLKGADPEDVIMGAFKVLDPDGKGSIKKSFLEELLTTQCDRFTPEEIKNMWAAFPPDVAGNVDYKNICYVITHGEDKEGE

SEQ ID NO: 159 - protein, 1940 aa, Gallus gallus
MATDADMAIFGEAAPYLRKSEKERIEAQNKPFDAKSSVFVVHAKESYVKSTIQSKESGKVTVKTEGGETLTVKEDQIFSMNPPKYDKIEDMAMMTHLHEPAVLYNLKERYAAWMIYTYSGLFCVTVNPYKWLPVYNPEVVLAYRGKKRQEAPPHIFSISDNAYQFMLTDRENQSILITGESGAGKTVNTKRVIQYFATIAASGDKKKEEQPAGKMQGTLEDQIISANPLLEAFGNAKTVRNDNSSRFGKFIRIHFGATGKLASADIETYLLEKSRVTFQLKAERSYHIFYQIMSNKKPELIEMLLITTNPYDYQYVSQGEITVPSINDQEELMATDSAIDILGFTPDEKTAIYKLTGAVMHYGNLKFKQKQREEQAEPGGTEVADKAAYLMGLNSADLLKALCYPRVKVGNEYVTKGQTVQQVYNSVGALAKSVFEKMFLWMVVRINQQLDTKQPRQYFIGVLDIAGFEIFDFNSLEQLCINFTNEKLQQFFNHHMFVLEQEEYKKEGIEWEFIDFGMDLAACIELIEKPMGIFSILEEECMFPKATDTSFKNKLYDQHLGKSNNFQKPKPGKGKAEAHFSLVHYAGTVDYNITGWLEKNKDPLNETVVGLYQKSSLKTLALLFASVGGAEAESGAGGKKGGKKKGSSFQTVSALFRENLNKLMSNLRSTHPHFVRCLIPNETKTPGAMEHELVLHQLRCNGVLEGIRICRKGFPIRILYADFKQRYKVLNASAIPEGQFIDSKKASEKLLGSIDVDHTQYKFGHTKVFFKAGLLGLLEEMRDEKLAQLITRTQARCRGFLMRVEFKKMMERRESIFCIQYNVRAFMNVKHWPWMKLFFKIKPLLKSAESEKEMANMKEEFEKTKEELAKSEAKRKELEEKMVSLLQEKNDLQLQVQAEADGLADAEERCDQLIKTKIQLEAKIKELTERAEDEEEMNAELTAKKRKLEDECSELKKDIDDLELTLAKVEKEKHATENKVKNLTEEMAALDETIAKLTKEKKALQEAHQQTLDDLQAEEDKVNTLTKAKTKLEQQVDDLEGSLEQEKKLRMDLERAKRKLEGDLKMTQESTMDLENDKQQLDEKLKKKDFEISQIQSKIEDEQALGMQLQKKIKELQARIEELEEEIEAERTSRAKAEKHRADLSRELEEISERLEEAGGATAAQIDMNKKREAEFQKMRRDLEEATLQHEATAAALRKKHADSTADVGEQIDNLQRVKQKLEKEKSELKMEIDDLASNMESVSKAKANLEKMCRSLEDQLSEIKTKEEEQQRTINDISAQKARLQTESGEYSRQVEEKDALISQLSRGKQAFTQQIEELKRHLEEEIKAKKCPAHALQSARHDCDLLREQYEEEQEAKGELQRALSKANSEVAQWRTKYETDAIQRTEELEEAKKKLAQRLQDAEEHVEAVNSKCASLEKTKQRLQNEVEDLMIDVERSNAACAALDKKQKNFDKILSEWKQKYEETQAELEASQKESRSLSTELFKMKNAYEESLDHLETLKRENKNLQQEISDLTEQIAEGGKAIHELEKVKKQIEQEKSELQTALEEAEASLEHEEGKILRVQLELNQVKSDIDRKIAEKDEEIDQLKRNHLRVVDSMQSTLDAEIRSRNEALRLKKKMEGDLNEIEIQLSHANRQAAEAQKNLRNTQGVLKDTQIHLDDALRSQEDLKEQVAMVERRANLLQAEIEELRAALEQTERSRKVAEQELLDASERVQLLHTQNTSLINTKKKLESDISQIQSEMEDTIQEARNAEEKAKKAITDAAMMAEELKKEQDTSAHLERMKKNLDQTVKDLQHRLDEAEQLALKGGKKQIQKLEARVRELEGEVDAEQKRSAEAVKGVRKYERRVKELTYQSEEDRKNVLRLQDLVDKLQMKVKSYKRQAEEAEELSNVNLSKFRKIQHELEEAEERADIAESQVNKLRAKSREIGKKAESEE

SEQ ID NO: 160 - protein, 1940 aa, Gallus gallus
MATDADMAIFGEAAPYLRKSEKERIEAQNKPFDAKSSVFVVHAKESYVKSTIQSKESGKVTVKTEGGETLTVKEDQIFSMNPPKYDKIEDMAMMTHLHEPAVLYNLKERYAAWMIYTYSGLFCVTVNPYKWLPVYNPEVVLAYRGKKRQEAPPHIFSISDNAYQFMLTDRENQSILITGESGAGKTVNTKRVIQYFATIAASGDKKKEEQPAGKMQGTLEDQIISANPLLEAFGNAKTVRNDNSSRFGKFIRIHFGATGKLASADIETYLLEKSRVTFQLKAERSYHIFYQIMSNKKPELIEMLLITTNPYDYQYVSQGEITVPSINDQEELMATDSAIDILGFTPDEKTAIYKLTGAVMHYGNLKFKQKQREEQAEPDGTEVADKAAYLMGLNSADLLKALCYPRVKVGNEYVTKGQTVQQVYNSVGALAKSVFEKMFLWMVVRINQQLDTKQPRQYFIGVLDIAGFEIFDFNSLEQLCINFTNEKLQQFFNHHMFVLEQEEYKKEGIEWEFIDFGMDLAACIELIEKPMGIFSILEEECMFPKATDTSFKNKLYDQHLGKSNNFQKPKPGKGKAEAHFSLVHYAGTVDYNITGWLEKNKDPLNETVVGLYQKSSLKTLALLFASVGGAEAESGAGGKKGGKKKGSSFQTVSALFRENLNKLMSNLRSTHPHFVRCLIPNETKTPGAMEHELVLHQLRCNGVLEGIRICRKGFPSRILYADFKQRYKVLNASAIPEGQFIDSKKASEKLLGSIDVDHTQYKFGHTKVFFKAGLLGLLEEMRDEKLAQLITRTQARCRGFLMRVEFKKMMERRESIFCIQYNVRAFMNVKHWPWMKLFFKIKPLLKSAESEKEMANMKEEFEKTKEELAKSEAKRKELEEKMVSLLQEKNDLQLQVQAEADGLADAEERCDQLIKTKIQLEAKIKELTERAEDEEEMNAELTAKKRKLEDECSELKKDIDDLELTLAKVEKEKHATENKVKNLTEEMAALDETIAKLTKEKKALQEAHQQTLDDLQAEEDKVNTLTKAKTKLEQQVDDLEGSLEQEKKLRMDLERAKRKLEGDLKMTQESTMDLENDKQQLDEKLKKKDFEISQIQSKIEDEQALGMQLQKKIKELQARIEELEEEIEAERTSRAKAEKHRADLSRELEEISERLEEAGGATAAQIDMNKKREAEFQKMRRDLEEATLQHEATAAALRKKHADSTAELGEQIDNLQRVKQKLEKEKSELKMEIDDLASNMESVSKAKANLEKMCRSLEDQLSEIKTKEEEQQRTINDISAQKARLQTESGEYSRQVEEKDALISQLSRGKQAFTQQIEELKRHLEEEIKAKNALAHALQSARHDCDLLREQYEEEQEAKGELQRALSKANSEVAQWRTKYETDAIQRTEELEEAKKKLAQRLQDAEEHVEAVNSKCASLEKTKQRLQNEVEDLMIDVERSNAACAALDKKQKNFDKILSEWKQKYEETQAELEASQKESRSLSTELFKMKNAYEESLDHLETLKRENKNLQQEISDLTEQIAEGGKAIHELEKVKKQIEQEKSELQAALEEAEASLEHEEGKILRVQLELNQVKSDIDRKIAEKDEEIDQLKRNHLRVVDSMQSTLDAEIRSRNEALRLKKKMEGDLNEIEIQLSHANRQAAEAQKNLRNTQGVLKDTQIHLDDALRSQEDLKEQVAMVERRANLLQAEIEELRAALEQTERSRKVAEQELLDASERVQLLHTQNTSLINTKKKLESDISQIQSEMEDTIQEARNAEEKAKKAITDAAMMAEELKKEQDTSAHLERMKKNLDQTVKDLQHRLDEAEQLALKGGKKQIQKLEARVRELEGEVDAEQKRSAEAVKGVRKYERRVKELTYQSEEDRKNVLRLQDLVDKLQMKVKSYKRQAEEAEELSNVNLSKFRKIQHELEEAEERADIAESQVNKLRAKSREIGKKAESEE

SEQ ID NO: 161 - protein, 615 aa, Gallus gallus
MKWVTLISFIFLFSSATSRNLQRFARDAEHKSEIAHRYNDLKEETFKAVAMITFAQYLQRCSYEGLSKLVKDVVDLAQKCVANEDAPECSKPLPSIILDEICQVEKLRDSYGAMADCCSKADPERNECFLSFKVSQPDFVQPYQRPASDVICQEYQDNRVSFLGHFIYSVARRHPFLYAPAILSFAVDFEHALQSCCKESDVGACLDTKEIVMREKAKGVSVKQQYFCGILKQFGDRVFQARQLIYLSQKYPKAPFSEVSKFVHDSIGVHKECCEGDMVECMDDMARMMSNLCSQQDVFSGKIKDCCEKPIVERSQCIMEAEFDEKPADLPSLVEKYIEDKEVCKSFEAGHDAFMAEFVYEYSRRHPEFSIQLIMRIAKGYESLLEKCCKTDNPAECYANAQEQLNQHIKETQDVVKTNCDLLHDHGEADFLKSILIRYTKKMPQVPTDLLLETGKKMTTIGTKCCQLGEDRRMACSEGYLSIVIHDTCRKQETTPINDNVSQCCSQLYANRRPCFTAMGVDTKYVPPPFNPDMFSFDEKLCSAPAEEREVGQMKLLINLIKRKPQMTEEQIKTIADGFTAMVDKCCKQSDINTCFGEEGANLIVQSRATLGIGA

SEQ ID NO: 162 - protein, 380 aa, Meleagris gallopavo
MPFSNSHNLLKMKHSADDEFPDLSAHNNHMAKVLTLDLYKKLRDRQTSSGFTLDDVIQTGVDNPGHPFIMTVGCVAGDEESYEVFKELFDPVIEDRHGGYKPTDEHKTDLNADNLQGGDDLDPNYVLSSRVRTGRSIRGFCFPHCSRGERRAIEKLSVEALGSLGGDLKGKYYALRNMTDAEQQQLIDDHFLFDKPVSPLLLASGMARDWPDARGIWHNDNKTFLVWINEEDHLRVISMQKGGNMKEVFTRFCTGLTQIETLFKSKNYEFMWNPHLGYILTCPSNLGTGLRAGVHIKLPNLGKHEKFGEVLKRLRLQKRGTGGVDTAAVGGVFDVSNADRLGFSEVELVQMVVDGVKLLIEMEKRLEKGQSIDDLMPAQK

SEQ ID NO: 163 - protein, 897 aa, Meleagris gallopavo; identical to SEQ ID NO: 149

SEQ ID NO: 164 - protein, 839 aa, Meleagris gallopavo
MAAPLSDGERRKQISVRGIAGLGDVAEVRKSFNRHLHFTLVKDRNVATPRDYFFALAHTVRDHLVGRWIRTQQHYYERDPKRIYYLSLEFYMGRTLQNTMVNLGLQNACDEAIYQLGLDLEELEEIEEDAGLGNGGLGRLAACFLDSMATLGLAAYGYGIRYEFGIFNQKIVDGWQVEEADDWLRYGNPWEKARPEYMLPVHFYGRVEHSPEGVKWVDTQVVLAMPYDTPVPGYKNNTVNTMRLWSAKAPNDFNLQEFNVGDYIEAVLDRNLAENISRVLYPNDNFFEGKELRLKQEYFVVAATLQDIIRRFKSSKFGCQDPVRTSFETFPDKVAIQLNDTHPALSIPELMRILVDVEKVDWDKAWEITKQTCAYTNHTVLPEALERWPVSMFEKLLPRHLEIIYALNQMHLDRVAALYPGDIDRLRRMSVIEEGDCKRINMAHLCVIGSHAVNGVARIHSDIVKNTVFKDFYELDPEKFQNKTNGITPRRWLLLCNPGLADVIAEKIGEDFVTDLSQLKKLLDFIDDETFIRDVAKVKQENKLKFSAYLEEHYKVKINPSSMFDIQVKRIHEYKRQLLNCLHAITLYNRKSDPSKSFVPRTIMIGGKAAPGYHMAKMIIKLITSIGEVVNNDPYVGDKLKVIFLENYRVSLAEKVIPAADLSQQISTAGTEASGTGNMKFMLNGALTIGTMDGANVEMAEEAGEENLFIFGMRVEEVEALDRQGYNAREYYDRIPELRQAIDQISSGFFSPRDPGCFRDVVNMLMHHDRFKVFADYEAYIKCQGQVDQLFMDPREWTKKVIRNIACSGKFSSDRTITEYAREIWGVEPSGDVFPAWEA

SEQ ID NO: 165 - protein, 364 aa, Meleagris gallopavo; identical to SEQ ID NO: 151

SEQ ID NO: 166 - protein, 192 aa, Meleagris gallopavo
MAPKKDVKKPAAAAAPAPAPAPAPAPAPAKPKEPAIDLKSIKIEFSKEQQDDFKEAFLLFDKTGDAKITLSQVGDIVRALGQNPTNAEMNKILGNPSKEEMNAKKITFEEFLPMLQAAANNKDQGTYEDFVEGLRVFDKEGNGTVMGAELRHVLATLGEKMTEEEVEELMKGQEDSNGCINYEAFVKHIMSV

SEQ ID NO: 167 - protein, 333 aa, Meleagris gallopavo; identical to SEQ ID NO: 153

SEQ ID NO: 168 - protein, 284 aa, Meleagris gallopavo
MEAIKKKMQMLKLDKENALDRAEQAEAEQKQAEERSKQLEDELAAMQKKLKGTEDELDKYSEALKDAQEKLELAEKKAADAEAEVASLNRRIQLVEEELDRAQERLATALQKLEEAEKAADESERGMKVIENRALKDEEKMELQEIQLKEAKHIAEEADRKYEEVARKLVIIEGDLERTEERAELAESKCSELEEELKNVTNNLKSLEAQAEKYSQKEDKYEEEIKILTDKLKEAETRAEFAERSVAKLEKTIDDLEDELYAQKLKYKAISEELDHALNDMTSI

SEQ ID NO: 169 - protein, 968 aa, Meleagris gallopavo
MCLQLFVFSELPPPFSFLKNKELTFVYCLHKALMVECWCHSICSSPTLADQIREVVGLESLVSSGKCILERIKHWLIIDIFLFLCISVWSIGESPPEETEKHDDSQRSTLFIEKPQSGSVSVGGNITFIAKVEAKDLLRKPNVKWFKGKWMDLASKAGKHLQLKESFERHTKIHTFEMHIIQAKENYAGNYRCEVSYKDKFDSCSFDLEVTESSQAAPSIDIRSAFKRSGDGQDDAGELDFSGLLKRREIKQQEEEPEVDVWELLKNANPSEYEKIAFQYGITDLRGMLKRLKRMRREVKKSAAFSKGLDPAYQVDKGGKVRFMVELADPTVELKWYKNGQEIRPSAKYIFEHKGNQRILFINNCTMTDDARYYVTAGDEKCSTELFVRDPPILVTKGLEDTNTYVGERVELSCEVSEDDANVKWFKNGVELTNEPKSRYRIKVEGKKHTLIIEEAAKNDNATYSVMTTGGQSEAKLSVDLRPLKISLALEDQTVRLGQEIHLKCEISENVEGKWYKNGQLIEASDRVKLYHKGRIHRFIIASAAVDDEGEYMFVPDAYNINIPCKVHVVDPPKLHLDGLGENNTVTVVAGTKLRLEIPITGEPTPKVMWSKGDKWITDSGRIRAETYSDSSCLVIDTAEREDSGPFRITLKNEAGEDSALINIKVVDVPDPPQAPNVTEVGEDWCVMTWEPPANDGGSPILGYFIERKKKQSSRWMRLNFELCKETTFEPKKMIEGVAYEVRVFAVNAIGTSKPSMPSKSFVPLAVTSPPTLLAVDSVTDTSVTMKWRPPDHIGAAGLDGYVVEYCFEGTDEWIVANPELTDKTKFTINGLPTGSKILVRVKAVNAAGESEPRYHPQPILVKEVIEPPKIRLPRHLKQTYTRRVGETVNLVIPFQVRTKNTDRKTLNTHFSYATASYLLFLYVNLGYWTAFVNILHPEGKSHDKASGIIHSYLGTKIGHYHMSSQSN

SEQ ID NO: 170 - protein, 1309 aa, Meleagris gallopavo
MNVKGEISAYASLVVKRYKGEIDASHLHAGTFSMPLSLGVTPHGFASRFEIKFIDKFEVMFGREGETMSLGCTVVISPDIKRFKPDIEWYRNGVLITPSKWVQMHWTGERASLTLTHANKEDEGLYTLRVVMGEYYEEYSGYVFVRDADAEIPGAPGAPLDVQCLDANKDYVIVSWKQPAVDGGNPILGYFIDRCEVGTTHWTQCNETPVKFARFPVTGLIEGRSYIFRVRAVNRAGVSLPSRVSEAVAALDPADRARLRSHPSAPWTGQIIVTEEEPAEGVIPGPPTDLQVIEATKNYVVLSWKPPGQRGHEGIMYFVEKCIAGTEDWQRVNTEIPVKSPRFAVFDLSEGKSYCFRVRCCNSAGIGEPSEATEATVVDDKLDIPKPPGRIMPTRNTDTSVVVTWTEPPDAKELVGYYIESSVVGSGHWEPCNNNPVKGTRFICHGLVNGEKYIFRVRAVNAAGLSEFSQDSEPIEVQAAIAAPSPPYDITVLESVRDSMVLGWKQPKATGGTEITGYYVNYREVIGGVPGKWREANIKAISERAYRIENLKENMVYQFQVAAANLAGVGTPSLPSKPFKCEEWTIAVPGPPHDVTYTEVRKDSLVLLWKEPVYTGRSPIAGYYIDMKETEAKEEHWRSVNEKPLQKKFLKIAGLTEGVSYVFRVRATNQAGVGRPSDITDPVVAETRPGTKEVVVNVDDDGVISLNFECDQMSPNSKFVWSKNYEPIEDDSRLDIDSKGGKSKAIFKNLGEDDLGIYSCVVTDTDGVSSGYTIDEEEMKRLLALSHERKFPTIPLASELAVEILEKGEVRFWLQAEKLSGNAKANFVFNDKEIFNGEKYKMKVDQRTGLVEMIMDKLEDKDEGTYTFQLQDGKATNQSSLVLIGDVFKKLRDEAEFQRKEWHRKQGPHFVDQLGWEVTDECNVMLKCKVANIKKETHIVWYKDDREIMVDEEHDFKDGVCTLLISEFSKKDTGIYEVILKDDRGKDVSELKLTDTAFADLMNEVCRRIALSATDLKIQSTAEGIRLYSFVTYYVDDLRVGWVHNDTQIRYTDRVKTGVTGEQIWLQINEPTPQDKGKYTMELFDGKTGHKKTVDLSGQAFDEAFAEFQRLKQAAIAEKNRARVLGGLPDVVTIQEGKALNLSCTVWGDPTPEVSWLKNEKSFVSDANCILKFEAGKNVSFTISAVSTHDSGKYSLVVKNKYGTETSDVTVIVITVSGQTDTAKHTVSISGQSQNNITVSKKKKATTMSKPQQEKEKKDIKPHVDEIIATEETSDSKEIPRVEKEKNTENNQQTAKKTHGKAAAARTVP

SEQ ID NO: 171 - protein, 434 aa, Meleagris gallopavo
MSILKIQAREIFDSRGNPTVEVDLYTNKGLFRAAVPSGASTGIYEALELRDNDKTRYLGKGVSKAVEHVNKTIAPALISKNVNVVEQEKIDKLMLEMDGTENKSKFGANAILGVSLAVCKAGAAEKGVPLYRHIADLAGNPEVILPVPAFNVINGGSHAGNKLAMQEFMILPVGADNFKEAMRIGAEVYHNLKNVIKEKYGKDATNVGDEGGFAPNILENKEALELLKTAIGKAGYSDKVVIGMDVAASEFYRDGKYDLDFKSPDDPSRYISPDQLADLYKGFVKNYPLVSIEDPFDQDDWAAWKKFTASVGIQVVGDDLTVTNPKRIAKAVEEKSCNCLLLKVNQIGSVTESLQACKLAQSNGWGVMVSHRSGETEDTFIADLVVGLCTGQIKTGAPCRSERLAKYNQLLRIEEELGSKARFAGRNFRNPRIN

SEQ ID NO: 172 - protein, 165 aa, Meleagris gallopavo
MAPKKAKKRIEGANSNVFSMFEQAQIQEFKEAFTIMDQNRDGFIDKADLRDTFAALGRLNVKNEEIDEMIKEAPGPINFTVFLTMFGEKLKGADPEETILNAFKVFDPEGKGLKSAYIKEMLMTQGERFSQEEIDQMFAAFPPDVSGNLDYKNLVHVITHGEEKD

SEQ ID NO: 173 - protein, 1974 aa, Meleagris gallopavo
MSSDAEMAIFGEAAPYLRKSEKERIEAQNRPFDAKAACFVVDQKQMYVKGTIQSREGGKVTVKTYDDTTVTVKDDEVFPMNPPKYDKIEDMAMMTHLHEPAVLYNLKERYAAWMIYTYSGLFCVTVNPYKWLPVYNPEVVSGYRGKKRQEAPPHIFSISDNAYQFMLTDRDNQSILITGESGAGKTVNTKRVIQYFATIAVTGEKKKDQQPSKMQGTLEDQIISANPLLEAFGNAKTVRNDNSSRFGKFIRIHFGTTGKLASADIETYLLEKSRVTFQLASERSYHIFYQIMSNKKPELIGEYLLLITTNPYDFTYVSQGEITVASIDDSEELLATDNAVDILGFSPDEKVGMYKLTGAVMHYGNMKFKQKQREEQAEPDGTEVADKAAYLMGLNSAELLKALCYPRVKVGNEYVTKGQNVQQVYNSVGALAKSVYEKMFLWMVTRINQQLDTKQPRQHFIGVLDIAGFEIFDFNSLEQLCINFTNEKLQQFFNHHMFVLEQEEYKKEGIDWEFIDFGMDLAACIELIEKPMGIFSILEEECMFPKATDTSFKNKLYDQHLGKSNNFQKPKPAKGKAEAHFSLIHYAGTVDYNITGWLEKNKDPLNETVVGLYQKSSLKLLSFLFSNYAGAETSDSGSGKKGGKKKGGSFQTVSAVFRENLNKLMTNLRSTHPHFVRCLIPNETKTPGEMDHYLVMHQLRCNGVLEGIRICRKGFPSRILYADFKQRYKILNASAIPEGQFIDSKKASEKLLSSIDVDHNQYRFGHTKVFFKAGLLGLLEEMRDEKLVSLITHMQAMCRGYLMRTEFKKMNARRESIYIIQYNVRAFMNVKHWPWMKLYFKMKPMLKSVESEKEMANMKEEFEKTKEELAKSEAKRKELEEKMVSLLQEKNDLQLQVQSESENLADAEERCEGLIKSKIQLEARIKELNERMEDEEEMNAELTAKKRKLEDECSELKKDIDDLELTLAKVEKEKHATENKVKNLTEEMAALDENISKLTKEKKALQEAHQQTLEYLQAEEDKVSILTKIKTKLEQQVDDLEGSLEQERKQRMDLERAKRKLDGDLKMSQDSIMDLKNDKQQTDERLKKKDFEISHLQSKIEDEQAQSSQLQKKIKELQARTEELEEEIEAERANCAKTEKQRADLSRELEEISERLEEAGGATAAQIEMNKKREAEFQKMRRDLEEATLQHEATAAALRKKHADSTAELGEQIDNLQRVKQKLEKEKSELKMEIDDLASNMESVSKAKSNLEKMCRALEDQYSEVRTKDDEHMRLINDMNTQKTRLQTENGELTRQLEEKESLISQLTRGKQAITQQTEELKRQLEEENKAKNALAHGLQSARHDCDLLREQYEEELEAKGELQRALSKANSEVAQWRTKYETDAIQCMEELEDAKKKLAQRLQDSEEQIEAVNSKCASLEKTKQRLQGEVDDLMIAVERSNAACAAFDKKQKNFDKVMAEWKQKYQESQAELEAAQKESRTLSTEIFRMKNAYEEVLDQVETVRRENKNLQQEISDLTEQLAEAGKANHGLEKAKKQTEQEKYDLQAALEEAEVSLEHEEGKILRVQLELNQVKSGVDRRSAEKDEEIQQLKKNHQRVLESMQTMLDAEIRSRNDALRLKKKMEGDLNDMEIQLSRANCQIAEIQKHLKGMQGQLKDSQLHLDDALRENDDLKEQLAVVERRNNLMMTELEQMRAALEQTERARKVSEQELTDTSERVQILHSQNTSLLNTKKKLEVDITHLQNEVEDSIQEASNAEEKAKKAITDAAMMAEELKKEQDTSAHLERMKRNLEQMVKDLQHRLDEAEQLTLKGGKKQLQKLEARIHELENELDIEQKRGTESLKGARKYERRLKELTYQSEEDKKNILRLQNLVDKLQLKVKAYKKQAEEAEEQASTNLSRCRKTQHELEEAEERADIAECQVNKLRAKSRDTGRQKSGLSSELGDNTFLPVIWHNMCNIDRPETTPQVILLHQ

SEQ ID NO: 174 - protein, 615 aa, Meleagris gallopavo
MKWVTLISFIFLFSSATSRNLQRVARDAEHKSEIAHRYNDLKEETFKAVAMITFAQYLQRCSYDGLSKLVKDVVDLAQKCVANEDAPECSKSLPSIILDEICQVEKLRDSYGAMADCCSKADPERNECFLSFKVSQPDFIQPYQRPASDVICKEYQDNRVSLLGHFIYSVARRNPYMYAPTILSFAADYEHALQSCCQESDVGACLDAKETAMREKAKKISVKQQYSCGILKKFGDRVFQAEKLVRLSQKYPKAPFSEVAKLVRESKDVHKECCEGDMVECMDDMAEIINNMCSKQDVFSSKIKGCCEKPIMERSQCIMEAEFDEKPADLPSLVEKYIQDKEVCKSFEAGHDAFMSEFVYEYSRRHPEFSTQLILRIAKGYESLLEKCCKTDNPAECYANAQDQLNQHIKETQDVVKTNCDLLSAHGEPDFLKSILIRYTKKMPQVSTETLLEIGKKMTAIGTKCCQLSEDRRMACSEGYLSIVIHDMCRRQETTPINDNVSHCCSDSYAHRRPCFTAMGVDTKYVPPPFNPDMFSFDEKLCSAPAEEREVGQMKLLINLIKRKPQMTEEQIKTIADGFTAMVDKCCKQSDINTCFGEEGANLIVQSRATLGIGV

SEQ ID NO: 175 - protein, 381 aa, Anas platyrhynchos
MPFSNSHNLLKMKHSATDEFPDLSAHNNHMAKVLTLDLYKKLRDKQTPSGFTLDDVIQTGVDNPGHPFIMTVGCVAGDEESYEVFKELFDPVIEDRHGGYKPTDQHKTDLNAGNLQGGDDLDPNYVLSSRVRTGRSIRGFCLPPHCSRGERRAIEKLSVEALGSLGGDLKGKYYALRNMTDAEQQQLIDDHFLFDKPVSPLLLASGMARDWPDARGIWHNDNKTFLVWINEEDHLRVISMQKGGNMKEVFTRFCTGLTQIENLFKSKNHEFMWNPHLGYILTCPSNLGTGLRAGVHIKLPHLGKHEKFGEVLKKLRLQKRGTGGVDTAAVGGVFDVSNADRLGFSEVELVQMVVDGVKLLIEMEKRLEKGQSIDDLIPAQK

SEQ ID NO: 176 - protein, 897 aa, Anas platyrhynchos
MNSMNQIETNMQYTYNYEEDEYMTQEEEWDRDLLLDPAWEKQQRKTFTAWCNSHLRKAGTQIENIEEDFRNGLKLMLLLEVISGERLPKPDRGKMRFHKIANVNKALDYIASKGVKLVSIGAEEIVDGNVKMTLGMIWTIILRFAIQDISVEETSAKEGLLLWCQRKTAPYRNVNIQNFHLSWKDGLGLCALIHRHRPDLIDYSKLNKDDPIGNINLAMEIAEKHLDIPKMLDAEDIVNTPKPDERAIMTYVSCFYHAFAGAEQAETAANRICKVLAVNQENERLMEEYERLASELLEWIRRTIPWLENRTPEKTMQAMQKKLEDFRDYRRKHKPPKVQEKCQLEINFNTLQTKLRISNRPAFMPSEGKMVSDIAGAWQRLEQAEKGYEEWLLNEIRRLERLEHLAEKFRQKASTHEQWAYGKEQILLQKDYESASLTEVRAMLRKHEAFESDLAAHQDRVEQIAAIAQELNELDYHDAASVNDRCQKICDQWDSLGTLTQKRREALERTEKLLETIDQLHLEFAKRAAPFNNWMEGAMEDLQDMFIVHSIEEIQSLISAHDQFKATLPEADGERQAILSIQNEVEKVIQSYSMRISASNPYSTVTVEEIRTKWEKVKQLVPQRDQSLQEELARQHANERLRRQFAAQANVIGPWIQTKMEEIARSSIEMTGPLEDQMNQLKQYEQNIINYKHNIDKLEGDHQLIQEALVFDNKHTNYTMEHIRVGWELLLTTIARTINEVETQILTRDAKGITQEQMNDFRASFNHFDRRKNGLMDHDDFRACLISMGYDLGEAEFARIMSLVDPNGQGTVTFQSFIDFMTRETADTDTAEQVIASFRILASDKPYILADELRRELPPEQAQYCIKRMPPYTGPGSVPGALDYTSFSSALYGESDL

SEQ ID NO: 177 - protein, 843 aa, Anas platyrhynchos
MAAPLSDGEKRKQISVRGIAGLGDVAEVRKSFNRHLHFTLVKDRNVATPRDYFFALAHTVRDHLVGRWIRTQQHYYERDPKRIYYLSLEFYMGRTLQNTMVNLGLQNACDEAIYQLGLDLEELEEIEEDAGLGNGGLGRLAACFLDSMATLGLAAYGYGIRYEFGIFNQKIVDGWQVEEADDWLRYGNPWEKARPEYMLPVHFYGRVEHTPEGVKWVDTQVVLAMPYDTPVPGYKNNTVNTMRLWSAKAPNDFNLQEFNVGDYIEAVLDRNLAENISRVLYPNDNFFEGKELRLKQEYFVVAATLQDIIRRFKSSKFGCRDPVRTSFETFPDKVAIQLNDTHPALSIPELMRILVDVEKVDWDKAWEITKRTCAYTNHTVLPEALERWPVSMFEKLLPRHLEIIYALNQMHLDRVAALYPGDVDRLRRMSVIEEGDCKRINMAHLCVIGSHAVNGVARIHSDIVKHSVFKDFYELEPEKFQNKTNGITPRRWLLLCNPGLADVIAEKIGEGFVTDLSQLKKLLDFIDNEMFIRDVAKVKQENKLKFSAYLEEQYKVKINPSSMFDVQVKRIHEYKRQLLNCLHAITLYNRIRSDPSKSFVPRTIMIGGKAAPGYHMAKMIIKLITSIGEVINNDPYVGDKLKVIFLENYRVSLAEKVIPAADLSQQISTAGTEASGTGNMKFMLNGALTIGTMDGANVEMAEEAGEENLFIFGMRVEDVEALDRKGYNALEYYERLPELRQAIDQISSGFFSPRDPGCFRDVVNMLMHHDRFKVFADYEAYIKCQGQVDQLFMDPREWTKKVIRNIACSGKFSSDRTITEYAQEIWGVEPSSTKIPPPNIPRD

SEQ ID NO: 178 - protein, 364 aa, Anas platyrhynchos
MTHQFPALSPEQKKALSDIAQRIVASGKGILAADESVGTMGNRLQRINVENTEENRRAFREILFSSDTSINQSIGGVILFHETLYQKDSSGKPFPAIIKEKGIVVGIKLDKGTSPLAGTNGETTIEGLDGLSERCAQYKKDGVDFGKWRAVLKITSTTPSQLAIQENANTLARYASICQQNGLVPIVEPEVLPDGDHDLQRCQYVTEKVLAAVYKALNDHHVYLEGTLLKPNMVTAGHACTKKYTPQDVAAATVTTLLRTVPAAVPGICFLSGGQSEEEASLNLNAMNQSSLPKPWKLTFSYGRALQASALAAWLGKSENKKAAQEAFRKRAQINSLACRGQYVVSGKTDAAATQSLFTASYTY

SEQ ID NO: 179 - protein, 192 aa, Anas platyrhynchos
MAPKKDVKKPAAAAAPAPAPAPAPAPAPAKPKEPAIDLKSIKIEFSKEQQDDFKEAFLLFDRTGDAKITLSQVGDIVRALGQNPTNAEINKILGNPSKEEMNAKKITFEEFLPMLQAAANNKDQGTYEDFVEGLRVFDKEGNGTVMGAELRHVLATLGEKMTEEEVDELMKGQEDSNGCINYEAFVKHILSV

SEQ ID NO: 180 - protein, 339 aa, Anas platyrhynchos
MVKVGVNGFGRIGRLVTRAAILSGKVQVVAINDPFIDLNYMVYMFKYDSTHGHFRGTVKAENGKLVINGNAITIFQERDPSNIKWADAGAEYVVESTGVFTTMEKAGAHLKGGAKRVIISAPSADAPMFVMGVNHEKYDKSLKIVSNASCTTNCLAPLAKVIHDNFGIVEGLMTTVHAITATQKTVDGPSGKLWRDGRGAAQNIIPASTGAAKAVGKVIPELNGKLTGMAFRVPTPNVSVVDLTCRLEKPAKYDDIKRVVKAAADGPLKGILAYTEDQVVSCDFNGDSHSSTFDAGAGIALNDHFVKLVSWYDNEYGYSNRVLALSTLLHAANIFPSSR

SEQ ID NO: 181 - protein, 284 aa, Anas platyrhynchos; identical to SEQ ID NO: 168

SEQ ID NO: 182 - protein, 1247 aa, Anas platyrhynchos
MPEPVKKTVSAFTKKPKTTEVTAGSTAVFEAETEKASVKVKWQRAGTEITDSEKYVIKAEGNKHSLTINNVVKEDDVTYAVIAGSSKVKFELKVKEPEKSETVTPAEAAPAPAASELPAPPAESSQNTEVPSAGTQPEEPADPIGLFVTRPQDGEVTVGGNITFTAKVAGESLLKKPSVKWFKGKWMDLASKVGQHLQLHDSYDRNNKVYTFELEIIEANMTFAGGYRCEVSTKDKFDSSNFNLTVNEAPLSGDLDIRAAFRRTTEGHDEAGELNFSALLKKRGDGKTESQPDVDVWEILRKAPPSEYEKIAFQYGITDLRGMLKRLKRIKKEEKKSTAFLKKLDPAYQVDKGQKIKLMVEVANPDADVKWLKNGQEIQVSGSRYIFEAVGNKRILTINHCSLADDAAYECVVGEEKSFTELFVKEPPILITRPLEDQMVMVGERVEFECEVSEEGATVKWEKDGVELTREETFKYRFKKDGKKQYLIINESTKEDSGHYTVKTNGGVSVAELIVQEKKLEVYQSIADLTVKARDQAVFKCEVSDENVKGIWLKNGKEVVPDERIKISHIGRIHKLTIEDVTPGDEADYSFIPQGFAYNLSAKLQFLEVKIDFVPREEPPKIHLDCLGQSPDTIVVVAGNKLRLDVPISGDPTPTVIWQKVNKKTDLLRSNDDSMTPSENSSDLNTDSKLLFESEGRVRVEKHEDHCVFIIEGAEKEDEGVYRVIVKNPVGEDKADITVKVIDVPDPPEAPKISNIGEDYCTVQWQPPKYDGGQPVLGYILERKKKKSYRWMRLNFDLLKELTYEAKRMIEGVVYEMRIYAVNSIGMSRPSPASQPFMPIAPPSEPTHFTVEDVSDSTVALKWRPPERIGAGGLDGYIVEYCKEGSTEWIPALPGLTERTSALIKDLVTGDKLHFRIKAINLAGESGAAIIKEPITVQEILQRPKIWLPRHLRQTLVKKVGETINIVIPFQGKPRPKITWTKDGQSLDAKDVGIRNSNTDTILFIRKAELHHSGAYEITLQIENMTDKVAITIQIIDKPGPPQNIKLTDVWGFNAALEWTPPQDDGNAQILGYTVQKADKKTMEWYTVFDHYRRTNCVVSELIMGNEYFFRVFSENLCGLSETAATTKNPAYIQKTGTTYKPPCYKEHDFSEPPKFTHPLVNRSVIAGYNTTLSCAVRGIPKPKIYWYKNKVDLSGDAKYRMFSKQGVLTLEIRKPTPFDGGFYTCKAVNECGEAEIECKLDVRVPQ

SEQ ID NO: 183 - protein, 1451 aa, Anas platyrhynchos
MSSLAVPFYQRRHKHFDQSYRNIQTRYVLEEYAARKAASRQAAYYESSGLGKTTCRLCARRARTLAHETMQESRKRTHEQKSHASEEKRIRFASELSALEKEIHTARHRAREQLDRLAIQRMVEENMAMERHAVEERISRAPEILVRLRSHTVWEKMSVSLCFTVQGFPTPVVQWYKNEELITPASDPAKYRVESKYGVHVLHINRADFDDSATYSAVATNVHGQASTNCAVVVRRFREGEEPHPAGIMPFHLPLSYDVCFTHFDVQFLEKFGVTFATEGETLTLKCSVLVTPELKRLRPRAEWYRDDVLIKDSKWTKLYFGEGQAALSFTHLNKDDEGLYTLRMITKGGVNECSAYLFVRDADALIAGAPGAPMDVKCHDANRDYVIVTWKPPNTTSQSPVIGYFVDKCEVGLENWVQCNDAPVKICKYPVTGLYEGRSYIFRVRAVNNAGISRPSRVSEPVAALDPVDLERTQTIHVDEGRKIVISKDDLEGDIQIPGPPTNVHASEISKTYVVLSWDPPVPRGREPLTYFIEKSMVGSGSWQRVNAQVAVKSPRYAVFDLAEGKPYVFRVVSANKHGISDPSEITEPIQPQDTVVVPSAPGRVVATRNTKTSVVVQWDKPKHEEDLYGYYIDYSVVGSNHWEPSNHKPIKYNRFVVHGLETGEQYIFRVKAVNAVGFSENSQESEAIKVQAALTCPSYPHGITLLNCDGHSMTLGWKAPKYSGGSPILGYYMDKREANHQNWHEVNSSLITRTIYTVEDLTEDAFYEFKIAAANIVGIGQPSDPSEHFKCKAWTMPEPGPAYDLTVCEVRNTSLVLLWKAPVYEGKSPITGYLVDYKEVDKEDWITANEKPTAQRYFKVTDLHEGHTYVFKVRAVNDAGVGKSSEVSEPVLVEAKPGTKEIFSGVDDEGNIYLAFECKEATDASHFAWGKSYEEIEDSDKYKIETEGDHSKLYFKNPDKSDLGTYSISVSDTDGVSSSFVMDDEELERLMAISNEIKNPTIPLKSELAYEVLEKGEVRFWIQAESLSPDSTFRFVINDKEVENSDRHKINCDRPNGIIEMVMDRFTIDNEGTYTVQIQDGKAKNQSSLVLIGDAFKAVLAESELQRKEFLRKQGPHFSEFLYWEVTEECEVLLACKVANTKKETVFKWYKDGSGIDVDEMPDLQKGECHLSIPRLSRKDEGVYKATLSDDRGHDVSTLELSGKVYDDIILALSRVSGKTASPLKILCTEEGIRLQCFLKYYNEEMKISWSHRESKISSGEKMKIGGGENIAWLQITEPTEKDKGNYTFEIFGDKESFKRTLDLSGQAFDDAYAEFQRLKAAAFAEKNRGKVIGGLPDVVTIMDGKTLNLTCTVFGNPDPEVVWFKNDKALELNEHYVVSLEQGKYASLTIKGVTSEDSGKFSIHVKNKYGGETVDVTVSVYRHGEKVPEVKPGQLAKPRPIPPSEDSS

SEQ ID NO: 184 - protein, 408 aa, Anas platyrhynchos
MPERSWIPVGTPLLRWTCTHTKACFEQRSPAVPPLVSTKHWSSETTTSPVSLEKGLSVVDQEKIDNLMLEMDGTENKSKFGANAILGVSLAVCKAGAAEKDVPLYRHIADLAGNSDLILPVPAFNVINGGSHAGNKLAMQEFMILPVGAESFRDAMRIGAEVYHNLKSVIKEKYGKDATNVGDEGGFAPNILENSEALELLKEAIDKAGYTDKIVIGMDVAASEFYRDGKYDLDFKSPDDPSRYISADELGDLYQSFVRDYPVVSIEDPFDQDDWEAWSKFTANVGIQIVGDDLTVTNPKRIERAVEEKACNCLLLKVNQIGSVTEAIQACKLAQENGWGVMVSHRSGETEDTFIADLVVGLCTGQIKTGAPCRSERLAKYNQLMRIEEELGDEARFAGHNFRNPSVL

SEQ ID NO: 185 - protein, 171 aa, Anas platyrhynchos
MQGENHRAPKKAKKRIEGANSNVFSMFEQAQIQEFKEAFTIMDQNRDGFIDKADLRDTFAALGRLNVKNEEIDEMIKEAPGPINFTVFLTMFGEKLKGADPEETILNAFKVFDPEGKGLKSAYIKEMLMTQGERFSQEEVDQMFAAFPPDVSGNLDYKNLVHVITHGEEKD

SEQ ID NO: 186 - protein, 1626 aa, Anas platyrhynchos
FSISDNAYQFMLTDRENQSILITGESGAGKTVNTKRVIQYFATIAASGEKKKEEQSGKMQGTLEDQIISANPLLEAFGNAKTVRNDNSSRFGKFIRIHFGATGKLASADIETYLLEKSRVTFQLKAERSYHIFYQIMSNKKPELIDMLLITTNPYDYHFVSQGEITVASINDQEELMATDSAIDILGFTADEKVAIYKLTGAVMHYGNLKFKQKQREEQAEPDGTEVADKAAYLMGLNSADLLKALCYPRVKVGNEYVTKGQTVQQVNNSVGALAKAVYEKMFLWMVVRINQQLDTKQPRQYFIGVLDIAGFEIFDFNSLEQLCINFTNEKLQQFFNHHMFVLEQEEYKKEGIEWEFIDFGMDLAACIELIEKPMGIFSILEEECMFPKATDTSFKNKLYDQHLGKSSNFQKPKPAKGKVEAHFSLVHYAGTVDYNITGWLEKNKDPLNETVIGLYQKSSVKTLALLFANYGGAEINPQKIIIKDTFKGSSFQTVSALFRENLNKLMTNLRSTHPHFVRCIIPNETKTPGAMEHELVLHQLRCNGVLEGIRICRKGFPSRVLYADFKQRYKVLNASAIPEGQFIDSKKASEKLLGSIDVDHTQYKFGHTKVFFKAGLLGLLEEMRDEKLAQLITRTQARCRGFLMRVEYQRMVERRESIFCIQYNVRAFMNVKHWPWMKLFFKIKPLLKSAESEKEMANMKEEFEKTKEELAKSEAKKKELEEKMVKLVQEKNDLQLQVQAEADALADAEERCDQLIKTKIQLEAKIKEVTERAEDEEEINAELTAKKRKLEDECSELKKDIDDLELTLAKVEKEKHATENKVKNLTEEMAALDETIAKLTKEKKALQEAHQQTLDDLQAEEDKVNTLTKAKTKLEQQVDDLEGSLEQEKKLRMDLERAKRKLEGDLKLSQDSIMDLENDKQQLDEKLKKKDFEISQIQSKIEDEQALGMQLQKKIKELQARIEELEEEIEAERTSRAKAEKHRADLSRELEEISERLEEAGGATAAQIEMNKKREAEFQKMRRDLEEATLQHEATAAALRKKHADSTAELGEQIDNLQRVKQKLEKEKSELKMEIDDLASNMESVSKAKANLEKMCRTLEDQLSEIKTKEEEHQRMINDLTAQRARLQTESGEYSRQVEEKDSQISQLSRSKQAFTQQIEELKRHLEEEIKAKNALAHALQSARHDCDLLREQYEEEQEAKGELQRALSKANSEVAQWRTKYETDAIQRTEELEEAKKKLAQRLQDAEEHVEAVNAKCASLEKTKQRLQNEVEDLMIDVERSNAACAALDKKQKNFDKILAEWKQKYEETQAELEASQKESRSLSTELFKMKNAYEESLDHLETLKRENKNLQQEISDLTEQIAEGGKAIHELEKVKKQIEQEKSEIQAALEEAEASLEHEEGKILRLQLELNQVKSEIDRKIAEKDEEIDQLKRNHLRIVESMQSTLDAEIRSRNEALRLKKKMEGDLNEMEIQLSHANRQAAEAQKNLRNTQGVLKDTQIHLDDALRTQEDLKEQVAMVERRANLLQAEIEELRAALEQTERSRKVAEQELMDASERVQLLHTQNTSLINTKKKLETDITQIQSEMEDAIQEARNAEEKAKKAITDASMMAEELKKEQDTSAH

SEQ ID NO: 187 - protein, 565 aa, Anas platyrhynchos
MITFAQYLQRCSYDGLSKLVKDVVDLAQKCVANEDAPECSKSLPSIFLDEICQVEKLRDSYGAMADCCAKSDPERNECFLSFKIPQPDFVQPYQRPASDVICKEYEDNRVSFLGHFIYSVARRNPFLYAPTILGVAADYEHALKSCCKESDVGACLDGKETGIREKVKKISVKQQYSCGILKKFGDRIFQADKLALLSQKYPKASFAEISKLIHDVKDVYKECCEGDMVECMDDRAELVNYMCSKHEAFSSKIKDCCEKPIVERSQCIMEAEFDEKPADLPSLVEKYIQDKEVCKSFEAGHDAFMSEFVYEYSRRHPEFSTQLILRIAKGYETLLEKCCKTDNPAECYANAQDQLNEHIKETQDVVKTNCDLLTTHGEADFLKALLIRYTKKMPQVSTETLLEIGKKMTGVGTKCCKLPEDRRLPCSEGYLSMVIQDMCRRQETSPINDNVSHCCSDSYAYRRPCFSAMGVDTKYVPPPFNPDVFNFDEKLCSAPAEERAEGQMKLLINLIKRKPQMTEEQIKTIADGFTAMVDKCCKQSDIETCFGEEGANLIVQSRATLGIGA

SEQ ID NO: 188 - protein, 418 aa, Cairina sp.
MCCLKCQQGFWDFCAPAVLARGPCFGCGALVTAVGSAMPFSNSHNLLKMKHSATDEFPDLSAHNNHMAKVLTLDLYKKLRDKQTPSGFTLDDVIQTGVDNPGHPFIMTVGCVAGDEESYEVFKELFDPVIEDRHGGYKPTDQHKTDLNAGNLQGGDDLDPNYVLSSRVRTGRSIRGFCLPPHCSRGERRAIEKLSVEALGSLGGDLKGKYYALRNMTDAEQQQLIDDHFLFDKPVSPLLLASGMARDWPDARGIWHNDNKTFLVWINEEDHLRVISMQKGGNMKEVFTRFCTGLTQIENLFKSKNHEFMWNPHLGYILTCPSNLGTGLRAGVHIKLPHLGKHEKFGEVLKKLRLQKRGTGGVDTAAVGGVFDVSNADRLGFSEVELVQMVVDGVKLLIEMEKRLEKGQSIDDLIPAQK

SEQ ID NO: 189 - protein, 885 aa, Cairina sp.
RAGDKPQDEYMTQEEEWDRDLLLDPAWEKQQRKTFTAWCNSHLRKAGTQIENIEEDFRNGLKLMLLLEVISGERLPKPDRGKMRFHKIANVNKALDYIASKGVKLVSIGAEEIVDGNVKMTLGMIWTIILRFAIQDISVEETSAKEGLLLWCQRKTAPYRNVNIQNFHLSWKDGLGLCALIHRHRPDLIDYSKLNKDDPIGNINLAMEIAEKHLDIPKMLDAEDIVNTPKPDERAIMTYVSCFYHAFAGAEQAETAANRICKVLAVNQENERLMEEYERLASELLEWIRRTIPWLENRTPEKTMQAMQKKLEDFRDYRRKHKPPKVQEKCQLEINFNTLQTKLRISNRPAFMPSEGKMVSDIAGAWQRLEQAEKGYEEWLLNEIRRLERLEHLAEKFRQKASTHEQWAYGKEQILLQKDYESASLTEVRAMLRKHEAFESDLAAHQDRVEQIAAIAQELNELDYHDAASVNDRCQKICDQWDSLGTLTQKRREALERTEKLLETIDQLHLEFAKRAAPFNNWMEGAMEDLQDMFIVHSIEEIQSLITAHDQFKATLPEADGERQAILSIQNEVEKVIQSYSMRISASNPYSTVTVEEIRTKWEKVKQLVPQRDQSLQEELARQHANERLRRQFAAQANVIGPWIQTKMEEIARSSIEMTGPLEDQMNQLKQYEQNIINYKHNIDKLEGDHQLIQEALVFDNKHTNYTMEHIRVGWELLLTTIARTINEVETQILTRDAKGITQEQMNDFRASFNHFDRRKNGLMDHDDFRACLISMGYDLGEAEFARIMSLVDPNGQGTVTFQSFIDFMTRETADTDTAEQVIASFRILASDKPYILADELRRELPPEQAQYCIKRMPPYTGPGSVPGALDYTSFSSALYGESDL

SEQ ID NO: 190 - protein, 843 aa, Cairina sp.
MAAPLSDGEKRKQISVRGIAGLGDVAEVRKSFNRHLHFTLVKDRNVATPRDYFFALAHTVRDHLVGRWIRTQQHYYERDPKRIYYLSLEFYMGRTLQNTMVNLGLQNACDEAIYQLGLDLEELEEIEEDAGLGNGGLGRLAACFLDSMATLGLAAYGYGIRYEFGIFNQKIVDGWQVEEADDWLRYGNPWEKARPEYMLPVHFYGRVEHNPEGVKWVDTQVVLAMPYDTPVPGYKNNTVNTMRLWSAKAPNDFNLQEFNVGDYIEAVLDRNLAENISRVLYPNDNFFEGKELRLKQEYFVVAATLQDIIRRFKSSKFGCRDPVRTSFETFPDKVAIQLNDTHPALSIPELMRILVDVEKVDWDKAWEITKRTCAYTNHTVLPEALERWPVSMFEKLLPRHLEIIYALNQMHLDRVAALYPGDVDRLRRMSVIEEGDCKRINMAHLCVIGSHAVNGVARIHSDIVKHSVFKDFYELEPEKFQNKTNGITPRRWLLLCNPGLADVIAEKIGEGFVTDLSQLKKLLDFIDNKMFIRDVAKVKQENKLKFSAYLEEQYKVKINPSSMFDVQVKRIHEYKRQLLNCLHAITLYNRIRSDPSKSFVPRTIMIGGKAAPGYHMAKMIIKLITSIGEVINNDPYVGDKLKVIFLENYRVSLAEKVIPAADLSQQISTAGTEASGTGNMKFMLNGALTIGTMDGANVEMAEEAGEENLFIFGMRVEDVEALDRKGYNALEYYERLPELRQAIDQISSGFFSPGDPGCFRDVVNMLMHHDRFKVFADYEAYIKCQGQVDQLFMDPWEWTKKVIRNIACSGKFSSDRTITEYAQEIWGVEPSSTKIPPPNIPRD

SEQ ID NO: 191 - protein, 364 aa, Cairina sp.
MTHQFPALSPEQKKALSDIAQRIVASGKGILAADESVGTMGNRLQRINVENTEENRRAFREILFSSDTSINQSIGGVILFHETLYQKDSSGKPFPAIIKEKGIVVGIKLDKGTSPLAGTNGETTIEGLDGLSERCAQYKKDGVDFGKWRAVLKITSTTPSQLAIQENANTLARYASICQQNGLVPIVEPEVLPDGDHDLQRCQYVTEKVLAAVYKALNDHHVYLEGTLLKPNMVTAGHACTKKYTPQDVAAATVTTLLRTVPAAVPGICFLSGGQSEEEASLNLNAMNQSSLPKPWKLTFSYGRALQASALAAWLGKPENKKAAQEAFRKRAQINSLACRGQYVVSGKTDAAATQSLFTASYTY

SEQ ID NO: 192 - protein, 333 aa, Cairina sp.
MVKVGVNGFGRIGRLVTRAAILSGKVQVVAINDPFIDLNYMVYMFKYDSTHGHFRGTVKAENGKLVINGNAITIFQERDPSNIKWADAGAEYVVESTGVFTTMEKAGAHLKGGAKRVIISAPSADAPMFVMGVNHEKYDKSLKIVSNASCTTNCLAPLAKVIHDNFGIVEGLMTTVHAITATQKTVDGPSGKLWRDGRGAAQNIIPASTGAAKAVGKVIPELNGKLTGMAFRVPTPNVSVVDLTCRLEKPAKYDDIKRVVKAAADGPLKGILAYTEDQVVSCDFNGDSHSSTFDAGAGIALNDHFVKLVSWYDNEYGYSNRVVDLMVHMASKE

SEQ ID NO: 193 - protein, 284 aa, Cairina sp.
MDAIKKKMQMLKLDKENALDRAEQAEADKKAAEERSKQLEDELVALQKKLKGTEDELDKYSESLKDAQEKLELADKKATDAESEVASLNRRIQLVEEELDRAQERLATALQKLEEAEKAADESERGMKVIENRAQKDEEKMEIQEIQLKEAKHIAEEADRKYEEVARKLVIIESDLERAEERAELSESKCAELEEELKTVTNNLKSLEAQAEKYSQKEDKYEEEIKVLTDKLKEAETRAEFAERSVTKLEKSIDDLEDELYAQKLKYKAISEELDHALNDMTSM

SEQ ID NO: 194 - protein, 1258 aa, Cairina sp.
YPTTKPKTTEVTAGSTAVFEAETEKAGVKVKWQRAGTEITDSEKYVIKAEGNKHSLTINNVVKEDDVTYAVIAGSSKVKFELKVKEPEKSETVTPAEAAPAPAASELPAPPAESSQNTEVPSAGTQPEEPVDPIGLFVTRPQDGEVTVGGNITFTAKVAGESLLKKPSVKWFKGKWMDLASKVGQHLQLHDSYDRNNKVYTFELEIIEANMTFAGGYRCEVSTKDKFDSSNFNLTVNEIYHLCHDIVSFPFPSKTNYIFLLLRSTEGHDEAGELNFSALLKKRGDGKTESQPDVDVWEILRKAPPSEYEKIAFQYGITDLRGMLKRLKRIKKEEKKSTAFLKKLDPAYQVDKGQKIKLMVEVANPDADVKWLKNGQEIQVSGRYIFEAVGNKRILTINHCSLADDAAYECVVGEEKSFTELFVKEPPILITHPLEDQMVMVGERVEFECEVSEEGATVKWEKDGVELTREETFKYRFKKDGKKQYLIINESTKEDSGHYTVKTNGGVSVAELIVQEKKLEVYQSIADLTVKARDQAVFKCEVSDENVKGIWLKNGKEVVPDERIKISHIGRIHKLTIEDVTPGDEADYSFIPQGFAYNLSAKLQFLEVKIDFVPREEPPKIHLDCLGQSPDTIVVVAGNKLRLDVPISGDPTPTVIWQKVNKKTDLLRSNDDSMTPSENSSDLNTDSKLLFESEGRVRVEKHEDHCVFIIEGAEKEDEGVYRVIVKNPVGEDKADITVKVIDVPDPPEAPKISNIGEDYCTVQWQPPKYDGGQPVLGYILERKKKKSYRWMRLNFDLLKELTYEAKRMIEGVVYEMRIYAVNSIGMSRPSPASQPFMPIAPPSEPTHFTVEDVSDSTVALKWRPPERIGAGGLDGYIVEYCKEGSTEWIPALPGLTERTSALIKDLVTGDKLHFRIKAINLAGESGAAIIKEPITVQEILQRPKIWLPRHLRQTLVKKVGETINIVIPFQGKPRPKITWTKDGQSLDAKDVGIRNSNTDTILFIRKAELHHSGAYEITLQIENMTDKVAITIQIIDKPGPPQNIKLTDVWGFNAALEWTPPQDDGNAQILGYTVQKADKKTMEWYTVFDHYRRTNCVVSELIMGNEYFFRVFSENLCGLSETAATTKNPAYIQKTGTTYKPPCYKEHDFSEPPKFTHPLVNRSVIAGYNTTLSCAVRGIPKPKIYWYKNKMDLSGDAKYRMFSKQGVLTLEIRKPTPFDGGFYTCKAVNERGEAEIECKLDVRGNHMSIMHNTNTAVK

SEQ ID NO: 195 - protein, 1451 aa, Cairina sp.
MSSLAVPFYQRRHKHFDQSYRNIQTRYVLEEYAARKAASRQAAYYESSGLGKTTCRLCARRARTLAHETMQESRKRTHEQKSHASEEKRIRFASELSALEKEIHTARHRAREQLDRLAIQRMVEENMAMERHAVEERISRAPEILVRLRSHTVWEKMSVSLCFTVQGFPTPVVQWYKNEELITPASDPAKYRVESKYGVHVLHINRADFDDSATYSAVATNVHGQASTNCAVVVRRFREGEEPHPAGIMPFHLPLSYDVCFTHFDVQFLEKFGVTFATEGETLTLKCSVLVTPELKRLRPRAEWYRDDVLIKDSKWTKLYFGEGQAALSFTHLNKDDEGLYTLRMITKGGVNECSAYLFVRDADALIAGAPGAPMDVKCHDANRDYVIVTWKPPNTTSQSPVIGYFVDKCEVGLENWVQCNDAPVKICKYPVTGLYEGRSYIFRVRAVNNAGISRPSRVSEPVAALDPVDLERTQTIHVDEGRKIVISKDDLEGDIQIPGPPTNVHASEISKTYVVLSWDPPVPRGREPLTYFIEKSMVGSGSWQRVNAQVAVKSPRYAVFDLAEGKPYVFRVVSANKHGISDPSEITEPIQPQDTVVVPSAPGRVVATRNTKTSVVVQWDKPKHEEDLYGYYIDYSVVGSNHWEPSNHKPIKYNRFVVHGLETGEQYIFRVKAVNAVGFSENSQESEAIKVQAALTCPSYPHGITLLNCDGHSMTLGWKAPKYSGGSPILGYYMDKREANHQNWHEVNSSLITRTIYTVEDLTEDAFYEFKIAAANIVGIGQPSDPSEHFKCKAWTMPEPGPAYDLTVCEVRNTSLVLLWKAPVYEGKSPITGYLVDYKEVDKEDWITANEKPTAQRYFKVTDLHEGHTYVFKVRAVNDAGVGKSSEVSEPVLVEAKPGTKEIFSGVDDEGNIYLAFECKEATDASHFAWGKSYEEIEDSDKYKIETEGDRSKLYFKNPDKSDLGTYSISVSDTDGVSSSFVMDDEELERLMAISNEIKNPTIPLKSELAYEVLEKGEVRFWIQAESLSPDSTFRFVINDKEVENSDRHKINCDRPNGIIEMVMDRFTIDNEGTYTVQIQDGKAKNQSSLVLIGDAFKAVLAESELQRKEFLRKQGPHFSEFLYWEVTEECEVLLACKVANTKKETVFKWYKDGSGIDVDEMPDLQKGECHLSIPRLSRKDEGVYKATLSDDRGHDVSTLELSGKVYDDIILALSRVSGKTASPLKILCTEEGIRLQCFLKYYNEEMKISWSHRESKISSGEKMKIGGGENIAWLQITEPTEKDKGNYTFEIFGDKESFKRTLDLSGQAFDDAYAEFQRLKAAAFAEKNRGKVIGGLPDVVTIMDGKTLNLTCTVFGNPDPEVVWFKNDKALELNEHYVVSLEQGKYASLTIKGVTSEDSGKFSIHVKNKYGGETVDVTVSVYRHGEKVPEVKPGQLAKPRPIPPSEGSS

SEQ ID NO: 196 - protein, 438 aa, Cairina sp.
MVFKMSILKIHAREIFDSRGNPTVEVDLYTNKGLFRAAVPSGASTGIYEALELRDNDKTRYMGKGVSKAVEHVNKTIAPALISKNVNVVEQEKIDKLMLDMDGSENKSKFGANAILGVSLAVCKAGAAEKGVPLYRHIADLAGNPEVILPVPAFNVINGGSHAGNKLAMQEFMILPVGADSFKEAMRIGAEVYHNLKNVIKEKYGKDATNVGDEGGFAPNILENKEALELLKTAIGKAGYSDKVVIGMDVAASEFYRDGKYDLDFKSPDDPSRYISPDQLADLYKGFVKNYPVVSIEDPFDQDDWAAWKKFTGSVGIQVVGDDLTVTNPKRIAKAVEEKSCNCLLLKVNQIGSVTESLQACKLAQSNGWGVMVSHRSGETEDTFIADLVVGLCTGQIKTGAPCRSERLAKYNQLLRIEEELGSKARFAGRNFRNPRIN

SEQ ID NO: 197 - protein, 165 aa, Cairina sp.
MAPKKAKKRIEGANSNVFSMFEQAQIQEFKEAFTIMDQNRDGFIDKADLRDTFAALGRLNVKNEEIDEMIKEAPGPINFTVFLTMFGEKLKGADPEETILNAFKVFDPEGKGLKSAYIKEMLMTQGERFSQEEVDQMFAAFPPDVSGNLDYKNLVHVITHGEEKD

SEQ ID NO: 198 - protein, 1945 aa, Cairina sp.
MTSPDAEMAAFGEAAPYLRKSEKERIEAQNKPFDAKSSVFVAHPKESFVKGTIQSKESGKVTVKTEGGETLTVKEDQIFSMNPPKYDKIEDMAMMTHLHEPAVLYNLKERYAAWMIYTYSGLFCVTVNPYKWLPVYNPEVVLAYRGKKRQEAPPHIFSISDNAYQFMLTDRENQSILITGESGAGKTVNTKRVIQYFATIAASGEKKKEEQSGKMQGTLEDQIISANPLLEAFGNAKTVRNDNSSRFGKFIRIHFGATGKLASADIETYLLEKSRVTFQLKAERSYHIFYQIMSNKKPELIDMLLITTNPYDYHFVSQGEITVASINDQEELMATDSAIDILGFTADEKVAIYKLTGAVMHYGNLKFKQKQREEQAEPDGTEVADKAAYLMGLNSADLLKALCYPRVKVGNEYVTKGQTVQQVNNSVGALAKAVYEKMFLWMVVRINQQLDTKQPRQYFIGVLDIAGFEIFDFNSLEQLCINFTNEKLQQFFNHHMFVLEQEEYKKEGIEWEFIDFGMDLAACIELIEKPMGIFSILEEECMFPKATDTSFKNKLYDQHLGKSSNFQKPKPAKGKAEAHFSLVHYAGTVDYNITGWLEKNKDPLNETVIGLYQKSSVKTLALLFANYGGAEAEASGGGASGGKKGGKKKGSSFQTVSALFRENLNKLMTNLRSTHPHFVRCIIPNETKTPGAMEHELVLHQLRCNGVLEGIRICRKGFPSRVLYADFKQRYKVLNASAIPEGQFIDSKKASEKLLGSIDVDHTQYKFGHTKVFFKAGLLGLLEEMRDEKLAQLITRTQARCRGFLMRVEYQRMVERRESIFCIQYNVRAFMNVKHWPWMKLFFKIKPLLKSAESEKEMANMKEEFEKTKEELAKSEAKRKELEEKMVKLVQEKNDLQLQVQAEADALADAEERCDQLIKTKIQLEAKIKEVTERAEDEEEINAELTAKKRKLEDECSELKKDIDDLELTLAKVEKEKHATENKVKNLTEEMASLDETIAKLTKEKKALQEAHQQTLDDLQAEEDKVNTLTKAKTKLEQQVDDLEGSLEQEKKLRMDLERAKRKLEGDLKLSQDTIMDLENDKQQLDEKLKKKDFEISQIQSKIEDEQALGMQLQKKIKELQARIEELEEEIEAERTSRAKAEKHRADLSRELEEISERLEEAGGATAAQVEMNKKREAEFQKMRRDLEEATLQHEATAATLRKKHADSTAELGEQIDNLQRVKQKLEKEKSELKMEIDDLASNMESVSKAKANLEKMCRTLEDQLSEIKTKEEEHQRMINDLTAQRARLQTESGEYSRQVEEKDSQISQLSRSKQAFTQQIEELKRHLEEEIKAKNALAHALQSARHDCDLLREQYEEEQEAKGELQRALSKANSEVAQWRTKYETDAIQRTEELEEAKKKLAQRLQDAEEHVEAVNAKCASLEKTKQRLQNEVEDLMIDVERTNAACAALDKKQKNFDKILAEWKQKYEETQAELEASQKESRSLSTELFKMKNAYEESLDHLETLKRENKNLQQEISDLTEQIAEGGKAIHELEKVKKQIEQEKSEIQAALEEAEASLEHEEGKILRLQLELNQVKSEIDRKIAEKDEEIDQLKRNHLRIVESMQSTLDAEIRSRNEALRLKKKMEGDLNEMEIQLSHANRQAAEAQKNLRNTQAVLKDTQIHLDDALRTQEDLKEQVAMVERRANLLQAEIEELRAALEQTERSRKVAEQELMDASERVQLLHTQNTSLINTKKKLETDIVQIQGEMEETIQEARNAEEKAKKAITDASMMAEELKKEQDTSAHLERMKKNLDQTVKDLQHRLDEAEQLALKGGKKQIQKLEARVRELEGDVDAEQKRSAEAVKGVRKYERRVKELTYQSEEDRKNILRLQDLVDKLQMKVKSYKRQAEEAEELSNVNLSKFRKIQHELEEAEERADIAESQVNKLRAKSREFHGKKIEEEE

SEQ ID NO: 199 - protein, 1945 aa, Cairina sp.; identical to SEQ ID NO: 198

SEQ ID NO: 200 - protein, 615 aa, Cairina sp.
MKWVTLISFIFLFSSATSRNLQRVARDADHKSEIAHRYNDLKEETFKAVAMITFAQYLQRCSYDGLSKLVKDVVDLAQKCVANEDAPECSKSLPSIFLDEICQVEKLRDSYGAMADCCAKSDPERNECFLSFKIPQPDFVQPYQRPASDVICKEYEDNRVSFLGHFIYSVARRNPFLYAPTILGVAADYEHALKSCCKESDVGACLDGKETAIREKVKKISVKQQYSCGILQKFGDRIFQADKLAFLSQKYPKASFTEISKLIHDVKDVYKECCEGDMVECMDDRAELVNYICSKHESFSSKIKDCCEKPIVERSQCIMEAEFDEKPADLPSLVEKYIQDKEVCKSFEAGHDAFMSEFVYEYSRRHPEFSTQLILRIAKGYETLLEKCCKTDNPAECYANAQDQLNEHIKETQDVVKTNCDLLTTHGEADFLKALLIRYTKKMPQVSTETLLEIGKKMTGVGTKCCKLPEDRRLPCSEGYLSMVIQDMCRRQETSPINDNVSHCCSDSYAYRRPCFSAMGVDTKYVPPPFNPEMFNFDEKLCSAPAEERAAGQMKLLVNLIKRKPQMTEEQIKTIADGFTAMVDKCCKQSDIETCFGEEGANLIVQSRATLGIGA

SEQ ID NO: 201 - protein, 381 aa, Salmo salar
MPFGNTHNNFKLNFKVEEEYPDLTKHNNHMAKVLTKDMYAKLRDKQTPSGFTLDDVIQTGVDNPGHPFIMTVGCVAGDEESYEIFKDLLDPIISDRHSGYKPTDKHKTDLNFENLKGGDDLDPNYVLSSRVRTGRSIKGYTLPPHNSRGERRAVEKLSVEALNTLDGEFKGKYYPLNKMTDAEQEQLIADHFLFDKPVSPLLLGAGMARDWPDARGIWHNDAKSFLVWVNEEDHLRVISMEKGGNMKEVFRRFCVGLKRIEETFKKHNHGFMWNEHLGYVLTCPSNLGTGLRGGVHVKLPKLSTHPKFEEILTRLRLQKRGTGGVDTASVGGVFDISNADRLGSSEVDQVQMVVDGVKLMVEMEKKLEKGEAIDGMIPAQK

SEQ ID NO: 202 - protein, 895 aa, Salmo salar
MTAIETQVQYGSYMTSEQVYMTQEDDWDRDLLLDPAWEKQQRKTFTAWCNSHLRKAGTQIENIEEDFRNGLKLMLLLEVISGERLPKPDKGKMRFHKIANVNKALDFICSKGVKLVSIGAEEIVDGNVKMTLGMIWTIILRFAIQDISVEETSAKEGLLLWCQRKTAPYRNVNVQNFHISWKDGLALCALIHRHRPDLIDYSKLRKDDPMGNLNTAFEVAEKYLDIPKMLDAEDIVNTPKPDEKAIMTYVSCFYHAFAGAEQAETAANRICKVLAVNQENEKLMEEYEKLASELLEWIRRTIPWLENRVAEQTMRAMQQKLEDFRDYRRVHKPPKVQEKCQLEINFNTLQTKLRLSNRPAFMPSEGKMVSDIANAWKGLEQVEKGYEEWLLTEIRRLERLDHLAEKFKQKSSLHESWTSGKVELLSMKDYESASLMEIRALMRKHEAFESDLAAHQDRVEQIAAIAQELNELDYHDAVTINARCQGICDQWDNLGTLTQKRRDSLERVEKLWETIDQLYLEFAKRAAPFNNWMDGAMEDLQDMFIVHSIEEIQSLITAHDQFKATLPEADKERVATMGIQNEIVKIAQTYGIKLSGVNPYTNLSPQDITDKWDAVKHLVPLRDQMLQEEVARQQSNERLRRQFAAQANIIGPWIQTKMEEISHVSVDIAGSLEEQMSNLKQYEQNIINYKCNIDKLEGDHQLSQESLIFDNKHTNYTMEHVRVGWEQLLTTIARTINEVENQILTRDAKGISQEQLNEFRASFNHFDRKRNGMMDPDDFRACLISMGYDLGEVEFARIMTLVDSNNTGVVTFQAFIDFMTRETAETDTADQVMASFKILASDKTYITVEELRRELPPEQADYCISRMTSYIGSGAPPGALDYISFSSALYGESDL

SEQ ID NO: 203 - protein, 843 aa, Salmo salar
MPKPLTDQDKRKQISVRGLAGVENVSDLKTNFNRHLHFTLVKDRNVATKRDYYFALANTVRDHLVGRWIRTQQHYYEKDPKRVYYLSLEFYMGRTLQNTMVNLALENACDEATYQLGLDMEELQDIEEDAGLGNGGLGRLAACFLDSMASLGLAAYGYGIRYEFGIFNQKIVNGWQVEEADDWLRYGNPWEKARPEYMRPVHFYGRVEHTPEGVKWVDTQVVLALPYDTPVPGYRNNVVNTMRLWSAKAPCDFNLKDFNVGGYIQAVLDRNLAENISRVLYPNDNFFEGKELRLKQEYFVVAATLQDIVRRFKSSKFGSTEVVRVDLSTLPDKVAIQLNDTHPAMAIPELMRILLDTEHQTWEKAWDICTRTCAYTNHTVLPEALERWPTDLLQNLLPRHLEIIYEINRRHLERIAKLYPGDHDRLRRMSLVEEGDQKKINMAHLCIVGSHAVNGVARIHSDILKATLFKDFYEVDPHKFQNKTNGITPRRWLVMCNPGLAEVIAERIGEDYIRDLDQLKKLLTFVDDDSLIHDIAKVKQENKLKFSAYLEEHYKVKINPNSMFDVQVKRIHEYKRQLLNCLHIITLYNRIKKEPNKNWTPRTIMIGGKAAPGYHTAKLIIRLITAIGEIVNHDTVVGDRLKVIFLENYKVTLAEKIIPASDLSEQISTAGTEASGTGNMKFMLNGALTIGTMDGANVEMAEEAGEENLFIFGMRVDDVDAMDKSGYDAMDYYNRIPELKQAMDQIAGGFFSPDQHDLFKDIVNMLLHHDRFKVFADYEAYIKCQEKVSALYKNPKEWTKMVIHNIAGCGKFSSDRTISQYAREIWGMEPSLERIPAPDDPRQ

SEQ ID NO: 204 - protein, 363 aa, Salmo salar
MPHSFPFLSTEQKKELSDIAQKIVAPGKGILAADESTGSVAKRFQSINAENTEENRRLYRQLLFTADRVEPCIGGVIFFHETLYQKTDDGKLFPQLIKERDMVVGIKVDKGVVPLAGTNGETTTQGLDGLYERCAQYKKDGADFAKWRCVLKITDTTPSELAIKENANVLARYASICQMHGIVPIVEPEILPDGDHDLKRCQYVTEKVLAAVYKALSDHHVYLEGTLLKPNMVTPGHSCSQKYSNQEIAMATVTALRRTVPPAVPGITFLSGGQSEEEASINLNAMNQCPLHRPWAITFSYGRALQASALKAWGGKPENGKACQEEFIKRALNNSKACQGKYVSSGDKGAAGGESLYVANHAY

SEQ ID NO: 205 - protein, 193 aa, Salmo salar
MAPKKDAKAPAKKAEPAKAAAPAPAPEPDVVAAPPPLDLSTVKVEFTPDQQEDYKEAFGLFDRVGDAKVAYNQVADIMRALGQNPTNKEVRKVLGNPSDEDMAAKRLEFEAFLPMLQHIVNDPNKGTFDDYVEGLRVFDKEGNGTVMGAELRIVLGTLGEKMSEAEIDALMQGQEDENGSINFEAFVKHIMSI

SEQ ID NO: 206 - protein, 334 aa, Salmo salar
MVKVGVNGFGRIGRLVTRAAFHSKKGVEIVAINDPFIDLDYMVYMFKYDSTHGRFHGEVKAEGGKLVIDGHKITVFHERDPANIKWGDAGATYVVESTGVFTTIEKASTHLKGGAKRVVISAPSADAPMFVMGVNHEKYDNSLKVVSNASCTTNCLAPLAKVIHDNYHIIEGLMSTVHAVTATQKTVDGPSGKLWRDGRGASQNIIPASTGAAKAVGKVIPELNGKITGMAFRVPTPNVSVVDLTVRLEKPASYDAIKKVVKAAADGPMKGILGYTEQQVVSSDFNGDTHSSIFDAGAGIALNDHFVKLVTWYDNEFGYSNRVIDLMAHMATKE

SEQ ID NO: 207 - protein, 284 aa, Salmo salar
MDAIKKKMQMLKLDKENALDRAEGAEGDKKAAEDKSKQLEDDLVALQKKLKGTEDELDKYSESLKDAQEKLEVAEKTATDAEADVASLNRRIQLVEEELDRAQERLATALTKLEEAEKAADESERGMKVIENRASKDEEKMELQDIQLKEAKHIAEEADRKYEEVARKLVIIESDLERTEERAELSEGKCSELEEELKTVTNNLKSLEAQAEKYSQKEDKYEEEIKVLTDKLKEAETRAEFAERSVAKLEKTIDDLEDELYAQKLKYKAISEELDNALNDMTSI

SEQ ID NO: 208 - protein, 1138 aa, Salmo salar
MPEAKKPAAKTDKAAAAPADKEARSAIEEGSECRDELPDDETVPGGQSELTGLFVERPESTTVIKGKNVTFVAKVDSSDLLRKPNMKWLKGKWLDLGSKAGKHVQFKEAYDRNSKVYTYEMSIIKVVEGDAGGYRCEVTSKDKCDSCTFEVTVEAVQEEQPANILDAFKRSGDAGEDAGDLDFSALLKKREKKARDEPKEDVDVWEILKDAKPCDYEKIAFDYGITDLRGLLKRLKKMKKVEPKKSDAFLKKLEQCYSVDKGKRTQMHVELHDPNVQVKWLKNGVEIKPSAKYVFECVGNKRTLTINKCNLSDDAAYECVVGEEKSFTEVFVKEPPVTITKLLDDVHVVVGEKVEFECEVSEEGANVKWMKDGVELTKDGKIRIKKDGKKHTLVINEATIEDIGMYYVYTNGGESKGELEVEAKELEVLQSIADLSVKACEQAMFKCEVSDEKVVGKWFKDGVEVKPGNRIKMSHIGRIHKLTIDDVKPQDEGNYTFVPEGYALSLSAKLNFIEIKIEYVPRQDPPKIHLDTSSTGSKNTIVVVAGNKLRLDVEITGEPVPTVCWMKGDTVISEAEGRVRVETRTTLSSFVIEGAERPDEGQYSIIVTNPAGEDRAELTVKIVDVPNPPENVRCMGVGEDTATITWDPPKFDGGAPIKGYLMERKKQGSSRWTKLNFEVFESTTYEAKKMIEGVFYEMRVFAVNGIGISQPSGNSKPFMPIAPTSEPTRLTVEDVTDSTCALKWRPPERVGAGGVDGYIIEWCKEGEDNWVAANKEPVDKNTYRVKGLPTGEKLLFRVVAMNIAGRSPPCTMKQSVTIREIMEYPKIRLPRQLRTKFIRKVGEKINLVIPFQGKPRPVVNWLKDGEPLENKSVGIRTSDFDTILFIRSAERDHSGTYTLSVQIDNMQDKADIHIQIVDKPGPPINVMVTDVWGFNAALEWKPPKDTGNTDITGYTIQKADKKTQGWFTVYEHNRRPSCTASDLVMGNEYSFRVFSENICGLSDEVAFSKNTAIIGKTDLEYNKPAFKERDMSSSPKFTAPLVDRVVVAGYSTAISCAVRAYPKAKIVWMKNKMIIGEDPKFLMQNNQGVLTLNIRKPGQFDGGKYSCKAINDLGEDEVECRLEVRVLKEKKEGDEEKK

SEQ ID NO: 209 - protein, 1502 aa, Salmo salar
MATKVMHFNQKKHVSHVSHHSSHTTKHVVKEQSSRKSSSKSVNQVQHTHATEAMAEPAYTVPAFRQRSADEIEEYQRVSSHVGKGLASIQKELHRMRVVTKTQVENIAIKREVQEMMHKKMTLSTETDKAPDFMVALRPHTVWEKTPVKLFCTVDGHPRPIVKWYKGGKPVDPLSAPGKYKIESKYGVHSLIISRCMVSDTAEYSAVATNSHGSTTSKASVIVKRPAGGAYGSCQLFGQVPHLTVIHHSKLEITMLDRFGVSFGTEGGSISLVCTMVVVPDLPNVPPLAQWYRDDKLLKAGKLAEIKVGGGAATLTLPHLAKDDEGLYTLRMWTKDGTTEHSAYLFVKDAAPSVAGAPGAPISVKAFDINSDYVLVAWKPPNTTNEAAITGYFVDKRESGSATWSQCNDAPVQICKYPVHGLNVGHAYHFRVRAVNSAGISRPSRESDKVTALDPAERERLQVIKLDGKHEVVIKDDDLEGYVTLPGEPTDMHASEIFKSYIVLSWKPPSPRGRAPLWYIIEKSVSGTGAWQRINTAVKLQSPRYPVFDLEAGKKYQFRVYSENLYGASEASKPTDPIEKVDEHGVPSAPGQVVATRNTKSSVFVQWDPPKHPNHLMGYYIDASLVGSKTWAPCNHKPYKNTRFVVHGLTPGETYVFRVQAVNVYGLSDESQESTPIAVEPALTTPSAPHGITMLSCDGASMIIAWNSPKHRGGSKINAYYVDKRDADSLAWKEVNSAPTNTRTYTVDGLTEGTFYEFKVQAGNLAGVGVPSAPSTPLKCEAWTSAVPGPAYDLAFREVRGDSLVILWKAPVYTGASAVTGYIVEMAKKGHHYHPLNEEAIGHCYLQVTGLEAGAEYTFKVKAVNAEGVGKASQTSEPVFAKALPGTQEIQCGVDEDTGDIFLSFEACEISETSNFAWSKNYKEISDCTRVSIETKGKHSKLTFVNPDVSDLGAFSVHVTDTDGVSASHTVTEDALNTMLELSYNIRHPIVPLKHQLNYEVLEKGHVRFWLQCLKMSPAVNYRFIVNDKEVTSSDSHKISHDVNTGVIQMTVDHFSKASEATYTVQIHDGRAKNQSSLVLVGDVFKAALKEAEFQRSEHIRKQGPHFAEYLSYHVDDDCTVLLVCKVANVKKETVFHWFKDEEEIVPETPPNVMSGSVALPISLFSRKDQGHYKAKLSDDRGKDTSVFDISGQVFDDIINAIAHIAGSSASELVLQCTPEGIRLQCYMKYYTEEIRTAWLHKDSKISSSEKMRIAGTAEMAWMQIREPSEKEKGHYSIQIMDATKSHTRTFDLSGQAYTDAYEEYLRLKAAAFAEKNRGRVVGGLPDVVTIMEQKTLSLTCTVWGDPSPEVTWFKNENEVVSTEHAKITLEANKFASLTITAVTSEDSGKYSINVRNKYGGEFVEITVSVYKQGESPPEPKMGGRGATPAPLASKTPAKTPTPAQTPTPSLKSPTPSLKSPTPSLRSPAKSPTPTPKSPTPPRRVKSPSPARSLKSPTPPRK

SEQ ID NO: 210 - protein, 434 aa, Salmo salar
MSITKIHAREILDSRGNPTVEVDLYTAKGRFRAAVPSGASTGVHEALELRDGDKSRYLGKGTVKAVDHVNKDIAAKLIEKKFSVVDQEKIDHFMLELDGTENKSKFGANAILGVSLAVCKAGAAEKGVPLYRHIADLAGHKDVILPCPAFNVINGGSHAGNKLAMQEFMILPIGASNFHEAMRIGAEVYHNLKNVIKAKYGKDATNVGDEGGFAPNILENNEALELLKTAIEKAGYPDKIIIGMDVAASEFYKAGKYDLDFKSPDDPARYITGDQLGDLYKSFIKGYPVQSIEDPFDQDDWAAWTKFTAAVDIQVVGDDLTVTNPKRIQQAVEKKACNCLLLKVNQIGSVTESIKACKLAQSNGWGVMVSHRSGETEDTFIADLVVGLCTGQIKTGAPCRSERLAKYNQLMRIEEELGAKAKFAGKDYRHPKIN

SEQ ID NO: 211 - protein, 170 aa, Salmo salar
MAPKKAKRRGAAAEGGSSNVFSMFEQSQIQEYKEAFTIIDQNRDGIISKDDLRDVLASMGQLNVKNEELEAMVKEASGPINFTVFLTMFGEKLKGADPEDVIVSAFKVLDPEATGFIKKEFLQELLTTQCDRFSAEEMKNLWAAFPPDVAGNVDYKQICYVITHGEEKEE

SEQ ID NO: 212 - protein, 1979 aa, Salmo salar
MTTLGLTEGHPAKYQHLTNLVPAISYELGQKKERVIMSTDAEMAAFGPAAIYLRKPERERIASQAAPFDAKTAFFVVEPKEMYLKGVLQSKEGGKATVKTLCNKVLTVKEDDIHPMNPPKYDKIEDMAMMTHLNEATVLYNLKERYAAWMIYTYSGLFCVTVNPYKWLPVYDSVVVNAYRGKKRIEAPPHIFSISDNAYQFMLTDHENQSILITGESGAGKTVNTKRVIQYFATIAVAGGKKEQTAAATSGKIKGSLEDQIIAANPLLEAYGNAKTVRNDNSSRFGKFIRIHFGTTGKLASADIETYLLEKSRVTFQLSAERSYHIFYQLMTGHQPQLLEALLITTNPYDYPIISHGEIAVKSIDDTEEFIATDTAIDILGFTAEEKIGIYKLTGSVMHHGAMKFKQKQREEQAEPDGTEEADKISYLMGLNSADLLKALCYPRVKVGNEMVTKGQTVPQVNNSTMALCKSVYEKMFLWMVVRINEMLDTKQSRQFFIGVLDIAGFEIFDYNSLEQLCINFTNEKLQQFFNHHMFVLEQEEYKKEGIEWAFIDFGMDLAACIELIEKPMGIFSILEEECMFPKASDTTFKSKLYDQHIGKTKAFEKPKPGKGKAEAHFALVHYAGTVDYNVTGWLDKNKDPLNDSVVQLYQKSSVKLLAMLYVGAAASQADDAARGGGGKKKKGGSFQTVSALFRENLGKLMTNLRSTHPHFVRCLIPNESKTPGLMENFLVIHQLRCNGVLEGIRICRKGFPSRIQYGDFKQRYRVLNASVIPDGQFIDNKKASEKLLGSIDVDKSQYKFGHTKVFFKAGLLGTLEEMRDEKLATLVTMTQALCRGYVMRKEFVKMMARREAIYSIQYNIRSFMNVKHWPWMKLYFKIKPLLKSAETEKEMAAMKENFEKMKEDLAKALAKIKGLEEKIVSLLQENNDLQLQIAAEENTLCDAEERCEGLIKSKIQMEAKLKETFERLEDEEELNAELTAKKRKLEDECSELKKDIDDLELTLAKVEKEKHATENKVKNLTEEMSSQDESLIKLTKEKKALQEVHQQVLDDLQAEEDKVNNLTKAKTKLEQQVDDLEGSLEQEKKVRMDLERAKRKLEGDLKLALESLMDLENDKQQSDEKIKKKDFETSQLLSKIEDEQALGAQLQKKIKELQARIEELEEEIEAERAARAKVEKQRSDLSRELEEISERLEEAGGATSVQIEMSKKREAEFQKLRRDLEESTLQHEATAAALRRKQADTVAELGEQIDNLQRVKQKLEKEKSEYKMEIDDLSSNMEAIAKAKGNLEKMCRSLEDQLSELKTKNDEHVRQLNDINVQRARLLTENGELGRQMEEKESLVSQLTRAKQTFTQQIEDLKRQIEEEVKAKNALAHGLQSARHDCDLLREQYEEEQEAKAELQRALSKANSEVAQWRSRYETDAIQRTEELEDAKKKLTQRLQDAQEAVEATNAKCASLEKTKQRLLGEVEDLMIDVERANASAAQLDKKQRNFDKVLSEWKQKYEEGQAELEGSLKEARSLSTELFKMKNSYEEALDHLETLKRENKNLQHEISDLTELVGESGKSIHELDKAKKTVENEKAEIQAALEEAEGTLEHEESKILRIQLELNQIKGEVDRKLAEKDEETEQIKRNSQRMMDSMQSTLDSEIRSRNDAMRVKKKMEGDLNEMEIQLSHANRQAAESQKQLRNVQGALKDAQLQLDDAIRVADDMKEQVAMVERRNNLMMAEIEELRAALEQTERGRKVAEQELVDASERVGLLHSQNTSLINTKKKLEVDLTQVQGEMEDTVQEARNAEEKAKKAITDAAMMAEELKKEQDTSSHLERMKKNLEVTVKDLQHRLDEAENLAMKGGKKQLQKLEARVRELESEVDAEQKRGVEAIKGVRKYERRVKELTYQTEEDNKNVSRLQDLVDRLQLKMKAYKRAAEDAEEQSNIHLSRYRKVQHELEEAQERADISESQVNKLRAKSREIGSKGNVAEE

SEQ ID NO: 213 - protein, 5447 aa, Salmo salar
MDENEEFEEEFEEEEIIEEEYFEETVTSTTSHQTTSRAAAVTQQPGVPVRRVVRKKVKVDTSKFMTPYLAHSQKMLDLFSYNKYRRAYDKAKGKPYAITADTPEMMRIKKAQEQLSEVKYRMEGNKVRCTSLYDGEAREIAHVKHVSELISKVLYRAKWDETKDRYLLPPDAPELVLAVKNAANYSKKLYTEQWDEDKTMCYPYSDSPELRRVAKAQEVLSDILYKKGHAERTAKYTSLAVPNDMELAKKNFEHRSDLKYHEDYNKNVKGHWCETPYIEVAVARVAMDNLSNKMYTAAYEDIKDQINFMQTETPEYKQHKKAGHAASSIKYKKDYENSKSVADYNVLPATDNPLLRQLRYAGTILSDKVYKASYEKSRGSSINYCDTPKFKMDSVVQQFSDAHYKDKYENEVKGHYIGSYEDIYMQHCKKMEEIKSEQNYKADYEDIKTRCFYPQTMSPEYEAHKKVAQCKDKVYRQHPDTVKFTQVTDSPVQLQAAINAKQLSDLNYHAAYELDKFKCALPPDYPFFIQSRANAYNLSDSCYRYDWEKVKAKQFEVKGDAISILAARAHTNIASDVKYKKEFEKNKGKMVGALSIHDDPKILHSMHVAKIQSDREYKKNYEKTKTKYITTLDMMSNTLAKKSQGIASMTGYRTIHNRYLLPHDAMSLDLAKKANIIQSDNEYHSDYNNSTKGTGWVAFGSMEVEKAKKAGDILNERKYRQHPDTVPFTSIADHPVMMQAQVNQIMRSDLHYKKGLEEIHQKYSLPHNDPQFLQAKCNAYNISKNYYKQSWEETIAKGYDMRPDAISVIHAKHGRHIASDIQYKKDYVKGKGKQIGCRNLQDDPLLVHYMEVARLQSDKNYKKDYHKSKLKYHSPVDMMSVVHAKHASTVQTFAGYRKIQHHYSLLPGAMNLELARTMNTNASDVNYKADWENSVKGTGWIPIGSIPVELAKQAGAVLDEHKYRQHPENFKFSSKMDGMNMELCKHNQKIMNVQEYKASGEKFLHTYNLPADAPEFMQAKCNAVNISKNYYTYAHRQDILKGHQVKADAIPILAARACTNIASNYKYKLAYEKGRGKHVGFRNVKDDPLLVHYMEVAKMQSEQNYKKDYHKSKLKYHSPVDMMSLVHAKHASAAQTFAGYRKSLHNYTLLPEAMPVQLARTMMEIQSNNAYKSDYNNMCRGSGWVPIGSMQVELAKTAMAARDEQGYRVHPSTLKFTSLTGQMNMALAMTNTKLLDDASYRASGEKFKHTYNLPADSPQFLQAKCNAQTMSESHYKHKWVEDIAKGYDIRPDAISVIHAKHGRHIASDIQYKKDYVKGKGKQIGCRNLQDDPLLVHYMEVAKMQSDKNYKKDYHKSKLKYHSPVDMMSLEHAKHASTVQTFAGYRKIQHHYTLTADSMNLALARTMNASSSDYEYKSDWNNSVRGTGWIPIGSLPVELAKVGGDIQSEQKYRTHPSKFQFKKLMDSMDIVLATDNNKIMNKQEYTKQWHNDKLTVHVMPDAPEIVLAKANAINMSQKTYKAGLAKMYKNGYDLKVDAIAIQAAKQGTKIASDYKYKLSHRMNRGKQIGCRNLQDDPLLVHYMEVARLQSDKNYKKDYHKSKLKYHSPVDMMSVVHAKHASTVQTFAGYRKIQHHYTVLADAMNVELAKQMQTIASDNLYKSDYETSVKGTGWIPIGSLDVEKAKTAGLIGSEKLYRQHPSSFKFTKDMHSMDVVLANANNAIMNKQTYVDAWNKDKTSVHIMPDSMEVVLARANKINCSATLYKLANEQDKKKGYDLRNDAIAILAAKQGTSIASDFKYRAGYRKQVGHHIGARCVEEDPLLMLAINSAKIASDALYKKDFHQSKTKFHLPLDMIAFELAKKNQIQVNHAKYVTRLHNWTCLPDSTDVVHARHVYDIASDAVYKADLKCLQGIGWVPIGSLDVEKAKKAGEVLSEKLYRQHPSNFKFTSSVQDIGMVLAKQNNDNANKQAYTEAWETDKTKNHIMPDAMDIVLANANKSNYSWNQYRKAMVEDKKKGYDMRNDAISIIAAKHGREIISDHKYKSSYRKQVGHHIGARCVEDDPLLVLALNSAKIASDALYKKDFHQSKTKFHLPLDMIAFELCKKNQIQVNDFNYRTHLHNWTCLPDSTDVVHARHVYDIASEAVYRADMKWLRGIGWVPTGSLDVEKAKKAGEILSEKKYRQHPSTLDFKSPLDGMDIVLAKHNGQIMNKRQYVEQWDADKIKIHVGNELPELVLAKANAITMSNKLYKQGFDETIKKGYYLPVDAISVKAAKHGREIISDYKYKAGYSKQVGHHIGARCVEEDPLLMLAINSAKIASDALYKKDFHQSKTKFHLPLDMIAFELCKKNQIQVNDFNYRTHLHNWTCLPDSTDVVHARRVYDLHSDAVYRADMEFIRGTGWVPIGSLDVLKAKKAAEILSDRLYKQKPDSLKYTTDMHSMPMVLAKANAEIINKGNYVAQWEADKIKIHVGNELPELVLAKANAINNSYKLYKAELEELYKKGYNLKSDCIAIKAAKHGREIISDYKYKAGYSEQVGHHIGARCVEEDPLIMLALNSAKIASDALYKKDFHQSKTKFHLPVDMLTIELAKKCQMQVNDFNYRTRLHNWTCLPDSSDVVQARKVYALHSDAVYRADMEWLKGTGWVPIGSIDVEKAKKAGEILSERKYREHPSNFKFTATTDDMPYALATANAQIMDKRQYVQQWEADKIQIHVGNELPELVLAKANAITMSNKLYREGFNEMIGQGYHLDKDAIAVQHAKKSREIISDYKYKAGFRNQCGHHIGAHCVEDDPLLVLAANAARISSDINYKKDFHLTKTKFSIPLDMMSFDLAKKSQFQIQEFTYRTKLHNWTCMPDNTDVVRARKVYSLHSDAVYRADLEWLRGCGWLPQGSVDVIKAKNAQTILNERLYRQPPSSIKFTSVVDSPEVVLSKQNADILSDRKYREVWDTEKTNIHLNNDLPSLVLSKANAIAISNKLYTKDWDADKAKGYHIKEDAIAVLKARKSRDIISDYKYHEGYRKQVGHHIGARCVEEDPLIMLALNSAKIASDALYKKDFNKSKTKFHLPVDMLTIELAKKCQVQVNDFNYRTHLHNWTCLPDSTDVVQARKTYDLQSDAVYKADLEWLRGCGWNPHESVEVLKVKHAQAVLADRGYRVKLDQQKFTAPVDRIDMVCAKNAAEILNEANYRALWHTEKTKYTITDTPGLATAREVAKNLHPKLYTKDWDKVKATGFFMPGDAVPIKQCIHNNKVQSDHKYKADYRKQAGHHIGARCVEDDPLIMLAINSAKIASDALYKKDFNKSKTKFHLPVDMLTIELAKKCQVQVNDDHYRTRLHNWTCLPDSTDVTQARKAYDLQSDVVYKADLEWLRGCGWVAADCVDHVKVRNAQKIMNERLYKKDAKDNFAKYTHIVDRPEIILATINAFNLSDLKYKETFNAEKGKYIGSEDTPQLAHSREVAKVISEKYYKLAWDESKATGYQLDAHYIPVVGAKKTREIVSDVKYHDTHEKAKGHYLAGTLVDFPEVMRCGEQEKQKGLRDYHKEYNTNKAKNHIPADIIPNMVAKRCQDMLSDVLYRTLLHNWTCHPDQQDIIRARKNNEILSDVYYKDDLNWIKGIGCYAWDTPELVRAKNAHLLQSDLKYKEEGKKEFNNYSIVTDTPVYVTALLGHTWASDLNYREAYHKDKHMYTTILDTYNYAHNATLKDIYSTKTYTSAWDKIKAKDYTLPSDSHALKHAKQQKVILSNVKYREDYEKFKSLYTLPKSLEDDPQTLRCLKAGKMGLDRLYKADYEKTKAKNHIPPDMLDVLSARQTQNYVSEIGYRKHLHQWICMPDMQVYVQARKVNEQLSDIFYKDDLNWLRGIGCYAWDTPEILRVKAADKLQSDNKYRAKGIEAFKDYSVVIDTPTYETAKLSSQNLSDLHYRNDYATNVKGTNSSPAVTVDTERARLANFNQSDNLYKEANKNFMPTGYSLPHDNPLMKQAKQNSIVASSVKYKNDYEKSKATNYTMDPDGVSFVNIRNANKVTNPRLYRELYEKGKGKIHSTYDTPEIRQVKMTQEAVSDLCYKEKYFANRGTMIPMGVTPQMIHCNHVNEITSDLRYKEDLLWLRGIGCFLYDTPEMSTVRNITKFRSNYEVEAKKNHANFTVVLDTPEYQRVTELKTHMSDLMYRAQGNVEKTKCTTTLDSLDVKRVKWAQQLSNKYMYTDLASKERAHFTPEKDTPIMSHARAMKVVFSEIKYKDQYEKMKHKYTSIHDTPILVRAKKAYLQSSDLRYKESFERSKGHYHSVKDALDIVCHRRVTDDISEVKYREKYINSLGTWRSMPDRPEFFHSRIIGENVSDIKYKEDLDWLKGIGCYVWDRPEMVQAEKNKVLFSERHYKETFEKNRHRFKYTCDTPFFQAAKKASVIINDRHYRAAYEKTKDKYSSVLDDPRNLLAKENAKNSQWKYREQYERAKNKFTSILETPEHESHKRSKAIGDNVYKMDYNKNKAKGYTLGHDTPHSTHMKKVKEITSVLKYKELYEKSKAQINMDPESFEIRAAKEAYKNISNLDYRKKYQATKNKWIWTVDRPDFIQAAKANFQQSDIEYTYDKEMMKGCVISIVDDKLAVLAKMNADLSNVKYREKFEKAKGQYKTVLDTPTNLHAKSVRILASESKYKLAGKMEQQTGQFTTLPTTRDTIHAKDMGKLVSEKQYKQKFEKEKGKSKYNSMRVPPDVQHAMDVAKNQSNFAYRQEAKAKLNYTSVAMRPDIMRATQASKLASNIGYREKARQEAAHGGSLVHRPDIALATAVSKLNSQVQYKAKYDKEKGLRPKYDMKESVVYKTQKDAHQLASEVKYKGDLKKLHKPVTDMAESLSMQHNLSTSKLSSDYQYKKKHEDSKGHYLMIADSPEQLHHKEASELQSHVKYKEKYEKAKGKAMLDFETPTYVTAKEAQHMQSAKDYHKEYEESIKGKNLSGLEVTPAMLHVRHATKIASERDYRRDLEEGVKGKGLTVFEETPELLRARNATAILSQKEYKKTLETEIKGKGMLALATDTPDFMRARNATDILSQQKYKQTAEADRASYTSVIDTPDILHAQNIRNMVSQKKYREEAEKCMSHYVPVLDTPEMMRVRENQKNFSTIKYKDLLCRCTAISDLPEVKRVREMQKNISSVLYKDSSAKGTPQVFTPEMERAKRNQEQISSVLYSDSFRKQVQGKAAFVLDTPEMRRVKETQRNISGVKYHQDFEKSKGSFTPTTSDPVMDRVKKNTQEFSDINYRGIQRRVVEMEKRRVVEHEQETVTDLRVWRTNPGSVFDYDPAEDNIQSRSLHMMSVQAQRRSKEHSRSTSAMSGVGDEKSEVSEDPAHHVSLYSNGFATSSMGYTHTKTVELQQRSSSVATQQTTVSSVPSHPSTTGKTVRAMYDYSSADMDEVSFKDGDVIVNVQSIDEGWMYGTVQRSGKTGMLPANYVEAI

SEQ ID NO: 214 - protein, 377 aa, Salmo salar
MCDDDETTALVCDNGSGLVKAGFAGDDAPRAVFPSIVGRPRHQGVMVGMGQKDSYVGDEAQSKRGILTLKYPIEHGIITNWDDMEKIWHHTFYNELRVAPEEHPTLLTEAPLNPKANREKMTQIMFETFNVPAMYVAIQAVLSLYASGRTTGIVLDSGDGVTHNVPIYEGYALPHAIMRLDLAGRDLTDYLMKILTERGYSFVTTAEREIVRDIKEKLCYVALDFENEMATAASSSSLEKSYELPDGQVITIGNERFRCPETLFQPSFIGMESAGIHETAYNSIMKCDIDIRKDLYANNVLSGGTTMYPGIADRMQKEITALAPSTMKIKIIAPPERKYSVWIGGSILASLSTFQAMWITKQEYDEAGPSIVHRKCF

SEQ ID NO: 215 - protein, 608 aa, Salmo salar
MQWLSVCSLLVLLSVLSRSQAQNQICTIFTEAKEDGFKSLILVGLAQNLPDSTLGDLVPLIAEALAMGVKCCSDTPPEDCERDVADLFQSAVCSSETLVEKNDLKMCCEKTAAERTHCFVDHKAKIPRDLSLKAELPAADQCEDFKKDHKAFVGRFIFKFSKSNPMLPPHVVLAIAKGYGEVLTTCCGEAEAQTCFDTKKATFQHAIAKRVAELKSLCIVHKKYGDRVVKAKKLVQYSQKMPQASFQEMAGMVDKIVATVAPCCSGDMVTCMKERKTLVDEVCADESVLSRAAGLSACCKEDAVHRGSCVEAMKPDPKPDGLSEHYDVHADIAAVCQTFTKTPDVAMGKLVYEISVRHPESSQQVILRFAKEAEQALLQCCDMEDHAECVKTALAGSDIDKKITDETDYYKKMCAAEAAVSDDNFEKSMMVYYTRIMPQASFDQLHMVSETVHDVLHACCKDEPGHFVLPCAEEKLTDAIDATCDDYDPSSINPHIAHCCNQSYSMRRHCILAIQPDTEFTPPELDASSFHMGPELCTKDSKDLLLSGKKLLYGVVRHKTTITEDHLKTISTKYHTMKDKCCAAEDQAACFTEEAPKLVSESAELVKV

SEQ ID NO: 216 - protein, 608 aa, Salmo salar
MQWLSVCSLLVLLSVLSRSQAQNQICTIFTEAKEDGFKSLILVGLAQNLPDSTLGDLVPLIAEALAMGVKCCSDTPPEDCERDVADLFQSAVCSSETLVEKNDLKMCCEKTAAERTHCFVDHKAKIPRDLSLKAELPAADQCEDFKKDHKAFVGRFIFKFSKSNPMLPPHVVLAIAKGYGEVLTTCCGEAEAQTCFDTKKATFQHAVMKRVAELRSLCIVHKKYGDRVVKAKKLVQYSQKMPQASFQEMGGMVDKIVATVAPCCSGDMVTCMKERKTLVDEVCADESVLSRAAGLSACCKEDAVHRGSCVEAMKPDPKPDGLSEHYDIHADIAAVCQTFTKTPDVAMGKLVYEISVRHPESSQQVILRFAKEAEQALLQCCDMEDHAECVKTALAGSDIDKKITDETDYYKKMCAAEAAVSDDSFEKSMMVYYTRIMPQASFDQLHMVSETVHDVLHACCKDEQGHFVLPCAEEKLTDAIDATCDDYDPSSINPHIAHCCNQSYSMRRHCILAIQPDTEFTPPELDASSFHMGPELCTKDSKDLLLSGKKLLYGVVRHKTTITEDHLKTISTKYHTMKEKCCAAEDQAACFTEEAPKLVSESAELVKV

SEQ ID NO: 217 - protein, 380 aa, Gadus morhua
MAKNCHNDYKMKFSGDEEFPDLSLHNNHMAKVLTKDIYNKLRGKSTPSGFTVDDIIQTGVDNPGHPFIMTVGCVAGDEESYEVFKDLLDPIISDRHSGYKPTDKHRTDLNFENLKGGDDLDPAYVLSSRVRTGRSIKGFTLPPHNSRGERRTIEKLSIEALATLSGEFKGKYYPLNGMTDKEQDQLINDHFLFDKPVSPLLTCAGMARDWPDARGIWHNDAKTFLVWVNEEDHLRVISMQQGGNMKEVFRRFCTGLLKIEETFKKHNHGFMWNEHLGYVLTCPSNLGTGLRGGVHVKLPKLSTHAKFEEILTRLRLQKRGTGGVDTASVGGVFDISNADRLGSSEVDQVQMVVDGVKLMVAMEKKLEKSESIDDMIPAQK

SEQ ID NO: 218 - protein, 892 aa, Gadus morhua
FHKLLQTNGKGMIMLAYMTQEEGWDRDLLLDPAWEKQQRKTFTAWCNSHLRKAGTQIENIEEDFRNGLKLMLLLEVISGERLPKPDKGKMRFHKIANVNKALDYICSKGVKLVSIGAEEIVDGNAKMTLGMIWTIILRFAIQDISVEETSAKEGLLLWCQRKTAPYRNVNVQNFHISWKDGLALCALIHRHRPDLIDYSKLRKDDPIGNLNTAFEVAEKFLDIPKMLDAEDIVNTPKPDEKAIMTYVSCFYHAFAGAEQAETAANRICKVLAVNQENEKLMEEYEKLASELLEWIRRTIPWLENRAAEQTMRAMQQKLEDFRDYRRVHKPPRVQEKCQLEINFNTLQTKLRLSNRPAFMPSEGKMVSDIANAWKGLEQVEKGYEEWLLTEIRRLERLDHLAEKFKQKCSLHETWTSGKEELLSQKDFESASLMEIRALMRKHEAFESDLAAHQDRVEQIAAIAQELNELDYHDAVTVNARCQGICDQWDNLGTLTQKRRDSLERVEKLWETIDQLYLEFAKRAAPFNNWMDGAMEDLQDMFIVHSIEEIQSLITAHDQFKATLPEADKERMATMGIQSEILKIAQTYGIKLSGINPYTNLSPQDISNKWDAVKHLVPLRDQMLQEEVARQQANERLRRQFAAQANIIGPWIQTKMEEIGHVSADITGSLEDQMNNLKQYEQNIINYKSNIDKLEGDHQLSQESLIFDNKHTNYTMEHIRVGWEQLLTTIARTINEVENQILTRDAKGISQEQLNEFRASFNHFDRKRNGMMDPDDFRACLISMGYDLGEVEFARIMTLVDANNTGVVTFQAFIDFMTRETAETDTAEQVMASFKILASDKSYITVDELRRELPLEQAEYCISRMTRYVGADGTTGALDYISFSSALYGESDL

SEQ ID NO: 219 - protein, 843 aa, Gadus morhua
MAKPLTDQEKRKQISVRGLAGVENVTDIKSNFNRHLHYTLVKDRNVATKRDYYFALANTVRDHLVGRWIRTQQHYYEKDPKRVYYLSLEFYMGRTLQNTMVNLALENACDEATYQLGLDMEELQDIEEDAGLGNGGLGRLAACFLDSMASLGLAAYGYGIRYEFGIFNQKIVDGWQVEEADDWLRYGNPWEKARPEYMRPVHYYGRVEHTPGGVKWVDTQVVLALPYDTPVPGYRNNVVNTMRLWSAKAPCDFNLKDFNVGGYIQAVLDRNLAENISRVLYPNDNFFEGKELRLKQEYFVVAATLQDIIRRFKSSKFGSTEFVRMDLSTLPDKVAIQLNDTHPALAIPELMRILMDLEELPWEKAWDIVVRTCAYTNHTVLPEALERWPIDLFQNLLPRHLDIVYEINRRHLERVAQQFPGDEGRLRRMSLIEEGDHKKINMAHLCIVGSHAVNGVARIHSEIIKDTVFKDFYEMDPEKFQNKTNGITPRRWLVMSNPGLAEVIAERIGDDYIRDLDQLKNLLEFVDDDSLIRDVAKVKQENKMKFSAYLEEHYKVKINPESMFDVQVKRIHEYKRQLLNCLHIITFYNRIKKQPNKAWTPRTIMIGGKAAPGYHTAKMIIKLITAIGDIVNHDAVVGDRLKVIFLENYKVTLAEKIIPASDLSEQISTAGTEASGTGNMKFMLNGALTIGTMDGANVEMAEEAGEENLFIFGMRVDDVKAMDQTGYDAKSYYDRIPELKQAMDQISGGFFSPGQPDLFKDIVNMLMNHDRFKVFADYEEYIKCQDKVSALYKDPKEWTKMVIRNIGSSGKFSSDRTIAQYAREIWGMEPSLEKIPAPDDPRD

SEQ ID NO: 220 - protein, 367 aa, Gadus morhua
MPHAYPFLSTEQKKELSDIAKKIVAPGKGILAADESTGSVAKRFQGINAENTEENRRLYRQLLFTADDRVAPCIGGVIFFHETLYQKTDDGKLFPQLIRERGQVVGIKVDKGVMPLAGTDGETTTQGLDGLYERCAQYKKDGADFAKWRCVLKITENTPSELAIMENANVLARYASICQMVRHHGIVPIVEPEILPDGDHDLQRTQYVTEKVLAAVYKALSDHHVYLEGTLLKPNMVTAGHSCTRKYSAQEVAMATVTALRRTVPPAVPGVTFLSGGQSEEQASINLNAINQCPLGRPWALTFSYGRALQASALKAWGGKKENGKACQEEYIKRALNNNLASQGKYVSSGDKGAAGGESLYVANHAY

SEQ ID NO: 221 - protein, 194 aa, Gadus morhua
MAPKKDVKAPAAAAKKAEPAKKAEPAPEPVAVPAPKTVDLSAVKVEFTPDQMEDYREAFGLFDRVGDNKVCYNQIADIMRALGQNPTNKEVKAILGNPSDEDMNSKRVDFEGFLPMMQTIVNSPNKGTLDDYVEGLRVFDKEGNGTVMGAELRIVLSTLGEKMTEAEIDALMQGQEDENGCINYESFVKHIMSI

SEQ ID NO: 222 - protein, 333 aa, Gadus morhua
MVKVGINGFGRIGRLVTRAGFQSKKVEIVAINDPFIDLEYMVYMFKYDSTHGRFHGEVKIEGDKLVIDGHKISVFHERDPANIKWGDAGAQYVVESTGVFTTIEKASAHLKGGAKRVIISAPSADAPMFVMGVNHEKYENSLQVVSNASCTTNCLAPLAKVIHDNFGIVEGLMSTVHAITATQKTVDGPSGKLWRDGRGASQNIIPASTGAAKAVGKVIPELNGKITGMAFRVPTPNVSVVDLTVRLEKPAKYDDIKKVIKAASEGPMKGILGYTEDQVVSTDFNGDIRSSIFDAGAGIALNDHFVKLVTWYDNEFGYSNRVIDLMAHMSCKE

SEQ ID NO: 223 - protein, 284 aa, Gadus morhua
MDAIKKKMQMLKLDKENALDRAEQAEGDKKAAEDRSKQLEDDLVALQKKLKGTEDELDKYSEALKDAQEKLELAEKKAADAESDVASLNRRIQLVEEELDRAQERLATALTKLEEAEKAADESERGMKVIENRAMKDEEKMELQEIQLKEAKHIAEEADRKYEEVARKLVIIESDLERTEERAELSEGKCSELEEELKTVTNNLKSLEAQAEKYSQKEDKYEEEIKVLTDKLKEAETRAEFAERSVAKLEKTIDDLEDELYAQKLKYKAISEELDHALNDMTSI

SEQ ID NO: 224 - protein, 1119 aa, Gadus morhua
MPEAADAGELPTAEEDSGDEEGQGTELTGLFVERPQENVVIIAGADVVITAKVDSTTLTRKPTVKWLKGKWLDLASKAGKHLQFKETYDRETKIYTYEMKVIKAKVGAAGAAGAYRCEVTSKDKFDSASFEITVEAAQQSDDVDILSSFKRADAGEDDGNLDFSALLKAAKKKKRPEDDKPVIDVWELLKSAHPSEYEKIAFEYGITDLRGMLKRLKKMKAEPKPSEAFLKRMESCYSVEKGKKIVLTCEVFDPNVQVKWLKNGQEIKGSAKYIMETIGNIRKLTIMKTTLADDAAYECVVGEDKCFTEVFVKEPPVTITKLMDDYHVVVGERVEFEVEVSEEGANAMWFFEDIEIFKDTNPAKYRIKKDGRKHMLIILEAGLEDIGMYHCWTNGGHTKGELEVEEKELLVLQDIADLTVKATDQAIFKCEVSDEKVTGKWFKDGVEVLPDERHKMTHIGRFHKLLIDNVKPQDAGDYTFIPNGYALSLSAKLNFLEIKIDYVPRQDPPKIHLDTTGNMVSQNTIIVVAGNKLRLDVEITGEPAPTVVWSKGEEVITATEGRVRVDNRKTLSSFVIEGAEREDEANYTICVTNPAGEDKAKLFVKIVDVPDPPQNVKCLSVGEDSSSMVWEPPAFDGGAPLKGYLMERKKKGSSRWTKLNFDVYEATTYEAKRMIEGVYYEMRVFAINSIGMSQPSITSKPFMPLAPTSEPTRLLVHDVTDGTCTLKWMAPERIGAGGLDGYIIEYCKEGETEWVVANKELCERQGYVVRGLPVGEKINFRVFAKNLAGVSPPATLAQPVTVREIVDLPKIRLPRDLRTKYIRKVGDKINLVIPFQGKPRPVVKWYKDGAPVDEKIVGVRNSTVDSILFIRSAERDHSGKYELVVQIENMEDRATIDLRVVERPGPPTKVKVTEVWGFNAALEWEAPKDDGNCEITGYTIQKADKKTMEWYTIYEHNRRPSCTSSDLIMGNEYMFRVYSENLVGPSEEPGQSKNTAVIGKTGLAYNPPEFKEKDVSCIPTFTQPLLDRSVVAGYSTAMSCSVKGFPKPKIVWMKNKMILGGDPKYIMLNNQGVLTLNIRKPSTFDGGRYSCMAVNDLGQALVECKLEIRIPTDPTAEVK

SEQ ID NO: 225 - protein, 1413 aa, Gadus morhua
MAMRSGNYNHEYYHKQSKYVVKEYSSSESVVDDYQHKAAVRIQGVVRKKMVEKYVREEPMIRGPKFVVRLRSHTVFENSPIKLFCTVEGFPVPIVTWYKDGVILEHGSAGRYIVDDNVGMRSLEIPRCGTDDTAQYSAVASNIHGQASSQASIIVKRFRQEDESCLYAWLPYTVPMLPSIQYTKINITFTEKFGPVFASEGGSASLTATMHLEPNLANLQPDSQWYRDDTRLVDSKWVKINTGRGTSTLTIPNLFKDDEGLYTLRLVTKGGTAEHSAFISVSDGPPPVVGAPGAPMDIRIHDANRDYVIVSWKPPNTTTEGPILGYFVDKCEVGTENWAQCNDHPIKICKYPVPGLFEGHSYYFRVRAVNARGISKPSRMSDPIAALDPTEFERLHALKLGGKLDVVTYDDDMEAEGKPPGVSSGIYASETDRTYVVLSWKAPAYSSKAPMWYYIEKCQVGTAAWQRVNTQVPIRSPRYAVFDLAEGKEYQFRVLSANMYGTSEPSEPSKLIQTQALKGVPSAPGQVISTRETDTSLLIQWAPPKEPNNLIGYYIDQCVKGSKDWTSANHKPHSSTKFVVSGLTTGETYVFRVQAINELGLSDESQESSPLSVKAALTSPTAPYDVALLGCDGQSMVLNWKKPLHTGGTAINAYFVDKRRSGATMWREVHIPAVTERVYQVEGLTQGAIYQFRVYAANLAGLGKSSKASADFSCEAWTMPEPGPAYDLTASEVRDTSLVLEWKKPVYYGSGPITGYHVEYAKKGSSDWTRANETAVSHRYLKVEGLEEGAKYVFRVRGVNEAGVGMASLPSDSVVAKALAGAQEVSCSVDEKSGDIVFAFESCQMNVGSEFLWKKNYKEITEFSSSIVVHTEGNLCKLMFKNAVKEDFGTYSVSVSHTDGVSSSCSISAEELQKMLELSYSIRHPIIPLKSELAYKILERGRMRFWLQAEEISSKVTYKFFANNKELSGADPNKMAHDVSTGVIEFILDHFTEDSEGTYTCQITDGGGKAQSSLVLIGDAFKAALAEADYQRREYIRVKEGPHFSEFLTVKVGEDCSVTLVCKVANLKKESEFHWYKEDTEIIPDVKPDLGSGVCKLPIHLFSRKTTGVYKATISDDRGKDMSQVDISGKVFDDVINKINQLAGASAAELVINCTATGIQLQCHMKYYTSEMNIHWMHGESKVGHSEKCVIGGTPAMATMEVIEPNDKDKGVYSIVITDLENTNKRTLDLSGDVYDKAYAQFQKLKAEAYAEMNRGKVMGGLPDVVTIMEKKTLSLTCTVCGDPKPLVSWLKNGNDVEPDNQYVVSLDQGKFACLTINGIGMDDSGKYTMIVQNKYGGECVDIVVSVYRHGDTIPVAKPNLTAKTIIPPKMPIELPQPKTAAPSPAPPASPSPAPPKAVGGRGMKSPTPTRRK

SEQ ID NO: 226 - protein, 457 aa, Gadus morhua
MKSNPLTSNTGEHASYLLHVNGVMSITKIHAREILDSRGNPTVEVDLWTAKGLFRAAVPSGASTGIHEALELRDGDKTRYLGKGTQKAVDHVNKDISPKLIEKNFSVVDQEKIDKFMLELDGTENKSKFGANAILGVSLAVCKAGAAEKGVPLYRHIADLAGNKDVILPVPAFNVINGGSHAGNKLAMQEFMILPVGAANFHEAMRIGAEVYHNLKNVIKAKYGKDATNVGDEGGFAPNILENNEALELLKSAIEKAGYPDKIIIGMDVAASEFYRSGKYDLDFKSPDDPARHISGKELGDLYKTFIANYPVQSIEDPFDQDDWANWADFTAAVDIQVVGDDLTVTNPKRIQQAVEKKACNCLLLKVNQIGSVTESIQACKLAQSNGWGVMVSHRSGETEDTFISDLVVGLCTGQIKTGAPCRSERLSKYNQLMRIEEELGDKAKFAGKDYRHPKIN

SEQ ID NO: 227 - protein, 170 aa, Gadus morhua
MAPKKAKRRAGGGDSGSSNVFSMFEQSQIQEYKEAFTIIDQNRDGIISKDDLRDVLASMGQINVKNEELEAMVKEASGPINFTVFLTMFGEKLKGADPEDVIVAAFKILDPEATGSIKKEFLQELLTTQCDRFSPEEIKNMWSAFPPDVAGNVDYKNICYVITHGEEKEE

SEQ ID NO: 228 - protein, 1935 aa, Gadus morhua
IMGDGEMQCFGEAAVYLRKPERERLEAQTSPFDAKTAFFVSEPKEMYLKGVLQSRDGGKATVKTLCDKTLTVKEEEIYPMNPPKYDKIEDMAMMTHLNEPTVLYNLKERYAAWMIYTYSGLFCVTVNPYKWLPVYDSSVVNAYRGKKRIEAPPHIFSISDNAYQAMLTDRENQSILITGESGAGKTVNTKRVIQYFATIAVSGPKKTEQVAGKMQGSLEDQIIAANPLLEAYGNAKTVRNDNSSRFGKFIRIHFATSGKLASADIETYLLEKSRVTFQLSAERSYHIFYQLMTGHKPELIDALLITKNPFDYHMISQGEITVKSIDDVEEFIATDDAIDILGFTADEKIGIYKLTGAVLHHGNMKFKQKQREEQAEPDGNEEADKIGYLMGLNSADVLKALCYPRVKVGNEYVTKGQTCPQVHNSVMALCKSVYEKMFLWMVVRINEMLDTKQPRQFFIGVLDIAGFEIFNYNSLEQLCINFTNEKLQQFFNHHMFVLEQEEYKKEGIEWEFIDFGMDLAACIELIEKPMGIFSILEEECMFPKASDTTFKNKLHDQHLGKTKAFEKPKPAKGKAEAHFSLVHYAGTVDYNITGWLDKNKDPLNDSVIQLYQKSSVKLLALLYASHALPDADAASKKGAKKKGGSFQTVSALFRENLGKLMTNLRSTHPHFVRCLIPNESKTPGLMENFLVIHQLRCNGVLEGIRICRKGFPSRVLYGDFKQRYKVLNASVIPEGQFIDNKKASEKLLGSIDVDSTQYKFGHTKVFFKAGLLGALEEMRDEKLAALVTLTQALCRGFNMRKEFVKMMERREAVFTIQYNIRSFMNVKTWPWMKVYFKIKPLLKSAETEKEMAQMKETFGKTKEDLAKALSKKKELEEKMVSLLQEKNDLLLQVQSEAETLSDSEERCEGLIKSKIQLEARLKETSERLEDEEEINSELTAKKRKLEDECSELKKDIDDLELTLAKVEKEKHATENKVKNLTEEMSSQDEIIAKLTKEKKALQEAHQQTLDDLQAEEDKVNTLSKAKCKLEQQVDDLEGSLEQEKKLRMDLERAKRKLEGDLKLAQESIMDLENDKQQSDEKIKKRDFETSQLLNKIEDEQTLSAQFQKKIKELQARIEELEEEIEAERAARAKVEKQRADLSRELEEISERLEEAGGATSLQIEMNKKREAEFQKLRRDLEESTLQHEATSAALRKKQADSVAELGEQIDNLQRVKQKLEKEKSECKMEIDDLSSNMEAVAKSKANLEKLARTLEDQLSELKSKNDEHVRQLNDLSVQRARLQTENGEFGRQQEEKEALISQLTRGKQAYTQQIEELKRHIEEEVKAKNALAHSVQSARHDCDLLREQFEEEQEAKAELQRGMSKANSEVAQWRSKYETDAIQRTEELEEAKKKLAQRLQDAEEAVEATNSKCASLEKTKQRLLGEVEDLMIDVERANALAANLDKKQRNFDKVLAEWKQKYEESQAELEGAQKEARSLSTEMFKLKNSYEESLDQLETLKRENKNLQQEISDLSEQMGETGKTIHELEKGKKTVEVEKSELQTSLEEAEATLEHEESKILRVQLELTQIKGEVDRKMAEKDEEMEQIKRNSLRVIDSMQSNLDSEIRSRNDALRIKKKMEGDLNEMEIQLSHANRQASEAQKQLRNVQGQLKDAQLHLDDALRGQEDMKEQVAMVERRNNLMLAEIEELRAALEQTERGRKVAEQELVDASERLGLLHSQNTSLINTKKKLETDLIQIQGEVEDTVQEARNAEEKAKKAITDAAMMAEELKKEQDTSSHLERMKKNLELTVKDLQHRLDEAESLAMKGGKKQLQKLEARVRELETECEAEQRRGSEAIKGVRKYERKVKELSYQCDEDKKNICRLQDLVDKLQLKMKAYKRQAEESEEQANTHLSRYRKVQHELEEAQERADVSESQVNKLRTKSRNGITKIIN

SEQ ID NO: 229 - protein, 6118 aa, Gadus morhua
MATAGMPTRKVRKRVAVDTSKFMTPYLAHSMKMLEQYSHNMYRKAYDLSRGTPPVHIADTPEMVRIRKAQEQLSEVKYRMEGNKARAGSLYDGEAKEIAHVKHMSELLSKVLYRQKWDETKDRYLLPPDAPELVLAVKNAANYSKKLYTEDWDLEKASFLPYSDSPELRRVAKAQEALSDIQYKKGYDQRKLKFTSLADRPDVELAKKVFTQRSDLKYKEDYNKNVKGQWCETPYFDVATARVAMENLSSRRYTQDWEAAKDQINFMQTETPVYDTNKKAGTSASSVKYRKDYEKTKDKSDYNTLPATENPLLRQLRYAGTILSDKVYKKNYEKSRGFSINYCDTPKFKMDSVLKKFTDTHYQDKYKNEVQGHYIGSYEDIYMLHCQKVEGFKSEQNYKAEYEDIKTRCFFPQTITPEYEVDKKLAQCKDKVYRQHPDKVKFTQVTDSPVQVQAALNAIQLSDINYKAKYEQTKTKNSLPADYPFFIQSRVNAFNLSENCYKYNWEKEKAKRFEMKGDAISILAARAHTNIASDVKYKKEYEKNRGRMVGALSIHDDPKILHSVHVAKIVSNREYTKDYEKVKTKYHTSLDMMSVTLAKKSQAIASMTGYKKISNRYLMPYDAILVDLAKKANSIQSDNEYKSDYNQYTKGSPWITFGSMDVELAKKAGEILSEKKYRKHPDTVPFTAIDDHPVMLQARVNQLQRSDLFYKKGLEEIHQKYSLPIDMPELQLARANSYNISKNYYKAAWEETISKGYDLKLDAISIKAAKAARHAASDIQYKKAYEKARGHHVGFRNIKDDPLLVHYMQVAKMQSEKNYKKDYHKSKLKYHSPVDMMSLVHAKQASKAQTYAGYRKIQHHYSLMPDAMNLVLARKMNTDASDCEYKSEWNNYVRGTGWVPIGSIGVETAKIGGQIQSENKYRTHPSTFKFSKLMDSMDLELATQAYTSAWEKDKRTIHVMPDSPEIVLAKANAITMSDVREYYYKLHQNGYHLKGDAIPIVAAKLGTKIASNYKYKLDYEKARGHHVGFRNIKDDPLLVHYMQVAKMQSEKNYKKDYEKAKLKYHSPVDMMSLVHAKEASKAQTYAGYRKIPHTYFLLPDNMQLQLCRKMQEIQSDNEYKSEYNNLYKGTGWVPIGSIDVELAKTANSALREHGYRQHPSSFKFTSKTDSMDMVLALANSKIIDQAAYKASGEKVKHTYHLPADAPEFIQAKFNAQTMSKSHYKQKWVEHIAKGYDMRPDAISILHAKQGRHIASDIQYKKAYEKARGHHVGFRSIKDDPLLVHYMQVAKMQSEKNYKKDYEKAKLKYHSPVDMMSLVHAKEASKAQTYAGYRKIQHHYNLMPDAMNLVLARKMNTDASDCEYKSEWNNYVRGTGWVPIGSIGVETAKIGGEIQSENKYRTHPSTFKFSKLMDSMDLELATANNKIMNKDKRTIHVMPDSPEIVLAKANAITMSDKLYKAGVMEMAKKGYHLKGDAIPIVAAKLGTKIASNYKYKLDYEKARGHHVGFRNIKDDPLLVHYMQVAKMQSERNYTKDYQKSKLKYHSPVDMMSLVHAKEASKAQTYAGYRKIEHHYNLMPDAMNLVLARKMNTDASDCEYKSEWNNYVRGTGWVPIGSIGVETAKIGGEIQSENKYRTHPSTFKFSKLMDSMDLELATANNKIMNKQAYTSAWEKDKRTIHVMPDSPEIVLAKANAITMSDKLYKAGVMEMAKKGYHLKGDAIPIVAAKLGTKIASNYKYKLDYEKARGHHVGFRCIQDDPLLVHYMQLAKQQSEKNYKKDYEKAKLKYHSPVDMMSLVHAKDATKAQTYAGYRKIQHHYSLLPDAMNLVLARNMQVNISDNQYKSDYESYCKGIGWIPIGSLDVEKAKIAGKIQSEKLYRQHPSNLKFTKDMFSMDLTLATANNQIMNKVLLALYCVDYTLMNILSSGNNLTNNFVVSFSKLYKLDNELSKKKGHNVRKDAIAILAAKNCTTIASDYKYKAGYRKQVGHHIGARCVEDDPLLVLALNSARIASDALYKKDFNKSKTKFNLPVDMMAFELAKKNQIQVNHANYITRLHNWTCLPDSTDVVNARRAYEIQSDNYYKEDQKLMQGIGWVPIGSLDVEKAKKAGEILSEHLYRKHPSNYKFTSKCDAMPLVLAKANAQITNKKAYHDAWDKEKSNIHIMPDAMDVVLAKANRANYSIKRYKQANEDAKKKGYDLRNDAIGIIAARAQRNIISDIKYKAGYRKQVGHHIGARCVEDDPLLMLALNSARIASDYLYKKDFNKSKTKFNLPVDMMAFELAKKNQIQVNHANYITRLHNWTCLPDSTDVVNARRAYEIQSDNYYKEDQKLMQGIGWVPIGSLDVEKAKKAGEILSEHLYRKHPSNYKFTSKCDAMPLVLAKANAQITNKKAYHDAWDKEKSNIHIMPDAMDVVLAKANRANYSIKGYKQANEDAKKKGYDLRNDAIGIIAARAQRNIISDIKYKAGYRKQVGHHIGARCVEDDPLLVLALNSARIASDYLYKKDFNKSKTKFNLPVDMMAFELAKKNQIQVNHANYITRLHNWTCLPDSTDVVNARRAYEIQSDNYYKEDQKLMQGIGWVPIGSLDVEKAKKAGQILSEHLYRKHPSNYKFTSKCDAMPLVLAKANAQITNKKAYHEAWDKEKSNIHIMPDAMDVVLAKANRANYSIKKYKEANEESKKKGYDLRNDAISIIAARAQRNIVSDYKYKAGYRKQTGHHIGARCVEDDPLLVLALNSARIASDALYKKDFNKSKTKFNLPVDMMAFELAKKNQIQVNHANYITRLHNWTCLPDSTDVVHARHVYEIQSDAVYKADLKWLQGMGWVPIGSLDVEKTKKAGEILSERKYRQPASSHKFTSPLDAMEIVLAKNNALTMNQRLYTEAWEKDKTKLHITSETPEIILSQQNAINMSNKKYKQGYEDTIKKGYYLPGDAISVKSAKHSRNIISDYKYKAGYRTQVGHHIGARCVEDDPLLMLALNSARIASDYLYKKDFNKSKTKFNLPVDMMAFELAKKNQIQVNHANYITRLHNWTCLPDSTDVVNARKAYDLQSDAVYKADMEFMKGIGWVPIGSLDVVKAKHAGKILSDHLYKQKTDKLKYTSDTKAMPMLLAKANAEVMNKKSYVAAWEKDKIKTHLIPDLPEILLSRSNAYNLSNKMYKEDVVKMFRKGYHLKGDAIAIVAAKKGREIISDIKYKAGYRKQVGHHIGALSVRDDPLIMLALNSARIASDALYKKDFNQSKTKFHLPVDMLTIELAKKCQVQVNDFNYRTRLHNWTCLPDSTDVVQARKAYDLQSDAVYKADMEWIKGQGWVPIGSLDVEKAKKAAEILSERKYREHPSKFKFTAKTDDMPYALAKANAEIMNKQSYVAAWESDKTKLHIMPDTPEIILGQQNKLNNSRKYYREAYEETLKKGYYLPKDAISVLAARASRNIISDYKYKAGFRKQCGHHIGALSVQDDPLLMMAANAAKIFSANLYKQDFHKTKTKYKLPVDMVAFDLAKTAQSHMKEVTYRTRLHNWTCMPDNTDVVHARKVYDLHSDAVYRADMEWIRGCGWSPQDSVDVVKARNAQKILNENLYRQAPSTVKFTSVVDLPAIVLSKQNAENISEMKYTAAWNKDKLTFHLKPDIPSVQLAKANAINISNKLYTKAWDNQKAKAYHIKEDTIAVLKARASRDIISDYKYKTGYRKQVGHHIGALSVRDDPLIMLALNSARIASDALYKKDFNQSKTKFHLPVDMLTIELAKKCQIQVNDFNYRTRLHNWTCLPDSTDVVQARKAYDLQSDAVYKADMEWIRGCGWVPHECVDVVKVKHAQKILADRGYRVKLDTQKFTAPVDRMDFVCAKNAAEVLNDAKYREAWHTDKSKFSLVDTPGLATSREVAKIMHPKLYTKDWDKVKATGYFMPGDAVPIKQCTQTTKVQSSHKYLEGYRKQVGHHIGALSVFDDPLIMLAMNSAKIASEALYKKDFNKSKTKFHLPVDMLTIELAKKCQIQVNDFNYRTRLHNWTCLPDSTDVVNARKAYDLQSDFVYKADLEWIRGCGWVPAESVHHVKVRKAQEILNERLYKKGAIENFSKYTHVVDRPELILARVAAANLSDLKYKETFNNEKGHYIGSEDTPQLAHSRECAKNISEAKYHESHEKAKGHYMANTLVDFPEVVRCGEQEKAKALRVYQKEYHESKTNNHIPSDIIANQVAKRCQDMLSDVIYRTHLHQWTCPPDQLDTVRARKGNEILSDVFYKEDLTWMKGMGCYVWDTPEIVRAKKSYELQSELKYKEAGKKEFNNYSIVTDTPVYVTAILGSTWASELNYREAYHKEKHLYTTILDTHDYARCHDFKHFFSNKIYTAAWDKIKAKGYKIPHDSHAMMHAKQQKVILSGVKYKEDYEKFKSLYSLPKCLQDDPATARCIKAGKLNLDRLYKEDYEKTKAKNHIPADMLDIVSGREVQRNVSEINYRKRLHQWVCLPDMQVFVQARKVNDQLSDIFYKDDLNYLRGIGCYAWDTPEILRVKTAGDQQSENKYKAKAVEAYKDYTVVLDTPTYETAKQNAVNLSDLHYRLDYNANVKGTNTAPAVTMETERARLANYNQSDNLYKEANKNSMATGYLMTNDTPVVKQVKNNSLVASSVKYKAAYEMMKATGYTLHPEGVTFVNTRKVNQVINPRLYREQYEKDKDKIHSTLDTPDIRQVKMNQEHLSELCYREKYNNTKGQLINMPMTPQLMHCQHVNQITSDLRYKEDLLWLRGLGCFMYDTPEMVNVRNITKFKNNYQVEAKKNHSKFSVVLDTPEYQRVTELKSHMSMVSPTSNTSLNTLDSVDIQRSKWAQQLSNKFLYTDLASKERAHFSQEVETPAMGLARTMKVTNSYNKYKEQYEKMKHRYTSIADTPILIRAKKAYLQSSDLRYKETFERSKGHYHTVKDALDILCHRRVTDDISEVKYREKYINSLGTWKSIPDRPEFFFNRIVGENVSSVKYKEDLEWMKGIGCFVWDTPEMVQADINKTLYSGRLYKASFEKNRYNFKYTCDTPFFHAAKNASSLINERSYRSSYEKNKDKFTITTDDPRYQLARENNKMSQAKYKSRAKELLKSGCNELHRPDILTALYQSVLGSKWKYREQYERVKSKFTSILETPEYESHKRSKKIGDIVYRMEYNKNKAKGYSLPYDTPHQNHMKKVQALTSNLKYREVYEKAKSQINMDPDAFEIRAAKEAYKNTTNLDYKKKYEATKNKWIWTVDRPDFISHSRNTLQQSDVEYKYDKEMMKGCVIPIVDDKITLLAKKGCEMASDIKYKEKYEKAKGHYNPVADSPQILHAKAVRTLASESKYKEASKKEMQSGSFTTLPETRDTVHSKEVNKLISGKLYKSKFEKEKGQSKYNRMIVPPDVQHAMDVAKSQSSYAYKRDAKDNLHYTTTADRPDIRKATQAAKLISDVGYREKARAEASRGGSLTYRPDIALATEVSKLTSLVDAKSALHGAASYDTLQMRHSKKMSALTSNVKYHEKFDKEMKGKKPKYDLKDSKIYQTLKDATNLASEVKYKGDLKKLHKPVTDMAESLSMQHNLSTSKLSSDYRYKKKFKDSKGHYLVIADTPEQLHHKEATELQSSVSGPSLCGVHVCKDYKKDYEEGMRGKNLCGLEVTPAMIHVRHATKIASEREYRKDLEEGVKGKGLSAPEETPEHLRAKNATHILSEREYKKSLELEIKGKGMLALATDTPDFMRARNATDILSQTKYKHTAELDRASFTSVIDTPDIIHAQQMRNIISQNKYKAEAGKTMSHYVPVFDTPEMQRVRENQKNFSTLQYQADHKIIKGKVLTVKDTPEMLRVKENTKNFSSVLYKDSKAKGTPVVFTPEMERVKRNQEHISSVLYSDSFRKQVQGKAAFVLDTPEMRRVKESQLNISGVRYHQDHEKSKGSFTAMASYPEMERVKKNTQEFSDINYRGIQRRVVEMERRRVVEHEQETTTDLRVWRTNPGSVFDYDPAEDNIQSRSLHMMSVHAQRRSKEHSRSTSAMSGPADEKSEMSHEGDHHMSTYSNGFGASSVGYQTTKTVELQQRSSSVATQQTTVSSIPSHPSTTGKTVRAMYDYGAADNDEVTFKDGDVIVNVQSIDEGWMYGTVQRTGKTGMLPANYVEAV

SEQ ID NO: 230 - protein, 1935 aa, Gadus morhua
MSTDAEMAVYGQAAIYLRKPEKERLEAQSKPFDAKSAAYVTDVKELYLKCTILKKEGGKVTVKVHDTEEEKTVKDDDVTPMNPPKYDKIEDMAMMTHLNEASVLYNLKERYAAWMIYTYSGLFCATVNPYKWLPVYDQHVVCAYRGKKRMEAPPHIFSVSDNAYQFMLTDRENQSVLITGESGAGKTVNTKRVIQYFATISVGGDKKKDIGAGKIQGSLEDQIIAANPLLEAYGNAKTVRNDNSSRFGKFIRIHFGTSGKLSSADIETYLLEKSRVTFQLPAERGYHIFYQMMTNHKPEIIEMTLITTNPYDFPTCSMGQIAVASIDDKEELVATDTAIDILGFNNDEKMGIYKLTGAVLHHGNMKFKQKQREEQAEPEGTEDADKVAYLLGLNSADMLKGLCYPRVKVGNEYVTKGQTVPQVLNSVSALAKSIYERMFLWMVIRINQMLDTKQPRSSFIGVLDIAGFEIFDYNSMEQLCINFTNEKLQQFFNHHMFVLEQEEYKKEGIIWEFIDFGMDLAACIELIEKPMGIFSILEEECMFPKASDVTFKAKLYDQHLGKTKAFEKPKPAKGKAEAHFSLVHYAGTVDYNITGWLDKNKDPLNDSVVQLYQKSGVKLLPVLYPPVASEDSTKKGGKKKGGSMQTVSSQFRENLGKLMTNLRSTHPHFVRCLIPNESKTPGLMENFLVIHQLRCNGVLEGIRICRKGFPSRILYADFKQRYKVLNASVIPEGQFIDNKKASEKLLGSIDVNHEEYKFGHTKVFFKAGLLGTLEEMRDEKLATLVGMIQALCRGFQMRKEFKKMMERRHAVYTIQYNIRSFMNVKTWPWMKVYYKIKPLLKSAETEKELSAMKENYEKMKKDLATALAKKKELEEKMVSLLQEKNDLSLQVASEGENLNDAEERCEGLIKSKIQLEARLKETTERLEDEEEMNADLTAKKRKLEDECSELKKDIDDLELTLAKVEKEKHATENKVKNLTEEMASQDESVAKLSKEKKALQEAHQQTLDDLQAEEDKVNTLSKAKTKLEQQVDDLEGSLEQEKKLRMDLERSKRKLEGDLKLAQESIMDLENDKQQSDEKIKKKDFETSQLLSKIEDEQSLAIQLQKKIKELQARIEELEEEIEAERAARAKVEKQRADLSRELEEISERLEEAGGATSAQIEMNKKREAEFQKLRRDLEESTLQHEATAAALRKKQADSVAELGEQIDNLQRVKQKLEKEKSEYKMEIDDLSSNMETVAKAKGNLEKICRTLEDQLSEIKAKSDENSRQINDIGAQRARLLTENGEFGRQLEEKEALVSQLTRGKQAYTQQIEELKRQIEEEVKAKNALAHGVQSARHDCDLLREQFEEEQEAKAELQRGMSKANGEVAQWRSKYETDAIQRTEELEESKKKLAQRLQEAEEQIEAVNSKCASLEKTKQRLQGEVEDLMVDVERANGLAANLDKKQRNFDKVLAEWKQKYEEGQAELEGTQKEARSLSTELFKMKNSYEEALDQLETLKRENKNLQQEISDLSEQIGETGKSIHELEKSKKQVETEKTEIQTALEEAEGTLEHEESKILRVQLELNQIKGEVDRKLAEKDEEMEQIKRNSLRITDSMQSTLDSEVRSRNDALRIKKKMEGDLNEMEIQLSHANRQAAEAQKQLRNVQGQLKDAQLHLDDAVRGGEDLKEQAAMVDRRNGLMVAEIEELRAALEQTERGRKVAEQELIDASERVGLLHSQNTSLLNTKKKLETDLVQVQGEVDDTVQEARNAEEKAKKAITDAAMMAEELKKEQDTSSHLERMKKNLEVTVKDLQHRLDEAENLAMKGGKKQLQKLESRVRELETEVEGEQRRGVDAVKGVRKYERRVKELTYQTEEDKKNGARLQDLVDKLQLKVKAYKRQAEEAEEQANSYLSKCRKVQHELEEAEERADIAETQVNKLRSKSRDGGKGKDPAE

SEQ ID NO: 231 - protein, 377 aa, Gadus morhua
MCDDEETTALVCDNGSGLVKAGFAGDDAPRAVFPSIVGRPRHQGVMVGMGQKDSYVGDEAQSKRGILTLKYPIEHGIITNWDDMEKIWHHTFYNELRVAPEEHPTLLTEAPLNPKANREKMTQIMFETFNVPAMYVAIQAVLSLYASGRTTGIVLDSGDGVTHNVPVYEGYALPHAIMRLDLAGRDLTDYLMKILTERGYSFVTTAEREIVRDIKEKLCYVALDFENEMATAASSSSLEKSYELPDGQVITIGNERFRCPETLFQPSFIGMESAGIHETAYNSIMKCDIDIRKDLYANNVLSGGTTMYPGIADRMQKEITALAPSTMKIKIIAPPERKYSVWIGGSILASLSTFQQMWISKQEYDEAGPSIVHRKCF

SEQ ID NO: 232 - protein, 377 aa, Thunnus maccoyii
MCDDDETTALVCDNGSGLVKAGFAGDDAPRAVFPSIVGRPRHQGVMVGMGQKDSYVGDEAQSKRGILTLKYPIEHGIITNWDDMEKIWHHTFYNELRVAPEEHPTLLTEAPLNPKANREKMTQIMFETFNVPAMYVAIQAVLSLYASGRTTGIVLDAGDGVTHNVPVYEGYALPHAIMRLDLAGRDLTDYLMKILTERGYSFVTTAEREIVRDIKEKLCYVALDFENEMATAASSSSLEKSYELPDGQVITIGNERFRCPETLFQPSFIGMESAGIHETAYNSIMKCDIDIRKDLYANNVLSGGTTMYPGIADRMQKEITALAPSTMKIKIIAPPERKYSVWIGGSILASLSTFQQMWISKQEYDEAGPSIVHRKCF

(19) United States
(12) Patent Application Publication
(10) Pub. No.: US 2025/0275551 A1
(43) Pub. Date: Sep. 4, 2025

(54) COMPOSITIONS AND METHODS FOR PRODUCING PROTEIN

(71) Applicant: Terraferma Foods, Inc., San Francisco, CA (US)

(72) Inventors: Ruby YU, San Francisco, CA (US); Victoria MO, San Francisco, CA (US)

(21) Appl. No.: 19/036,854

(22) Filed: Jan. 24, 2025

Related U.S. Application Data
(63) Continuation of application No. PCT/US2023/028715, filed on Jul. 26, 2023.
(60) Provisional application No. 63/392,613, filed on Jul. 27, 2022.
(60) Provisional application No. 63/402,544, filed on Aug. 31, 2022.

(51) Int. Cl. A23J 3/04; A23K 20/147; A23K 50/40; C12P 21/02

(52) U.S. Cl. CPC A23J 3/04; A23K 20/147; A23K 50/40; C12P 21/02

(57) ABSTRACT

Disclosed herein is a food product comprising an animal-free composition comprising a meat protein.

### CROSS-REFERENCE

This application is a continuation of International Application No. PCT/US2023/028715, filed Jul. 26, 2023, which claims the benefit of U.S. Provisional Application No. 63/392,613, filed Jul. 27, 2022, and U.S. Provisional Application No. 63/402,544, filed Aug. 31, 2022, each of which is entirely incorporated herein by reference.

### SEQUENCE LISTING

The instant application contains a Sequence Listing which has been submitted electronically in XML format and is hereby incorporated by reference in its entirety. Said XML copy, created on Jan. 21, 2025, is named 63116-701_601_SL.xml and is 458,343 bytes in size.

### BACKGROUND

Livestock is the world's largest user of land resources on Earth. Land dedicated to the production of meat and dairy, which includes animal feed production, encompasses nearly 80% of global agricultural land. One-fifth of the word's meat production is consumed by companion animals (e.g., pets). Recombinant protein production can be used to supplement or offset meat production.

### SUMMARY

An aspect of the present disclosure provides a method for producing a food product, the method comprising: a) contacting a plurality of Pichia cells with a recombinant nucleic acid; b) expressing a protein encoded by the recombinant nucleic acid; and c) isolating the protein from the plurality of Pichia cells, wherein the protein comprises an amino acid sequence that is at least 80% identical to any one of SEQ ID NOs: 54-232.

In some embodiments, the food product is a cat food product. In some embodiments, the protein is an albumin protein. In some embodiments, the albumin protein is a rabbit albumin protein. In some embodiments, the albumin protein is a duck albumin protein. In some embodiments, the duck albumin protein is a mallard duck albumin protein. In some embodiments, the duck albumin protein is a Muscovy duck albumin protein. In some embodiments, the albumin protein is a cow albumin protein. In some embodiments, the albumin protein is a sheep albumin protein. In some embodiments, the albumin protein is a pig albumin protein. In some embodiments, the albumin protein is a bison albumin protein. In some embodiments, the albumin protein is a deer albumin protein. In some embodiments, the albumin protein is a chicken albumin protein. In some embodiments, the albumin protein is a turkey albumin protein. In some embodiments, the albumin protein is a salmon albumin protein. In some embodiments, the albumin protein is a cod albumin protein. In some embodiments, the albumin protein is a tuna albumin protein.

In some embodiments, the Pichia cells are Pichia pastoris cells. In some embodiments, the protein comprises an amino acid sequence that is at least 90% identical to any one of SEQ ID NOs: 54-232. In some embodiments, the protein comprises an amino acid sequence that is at least 95% identical to any one of SEQ ID NOs: 54-232. In some embodiments, the food product is substantially animal-free.

Another aspect of the present disclosure provides a food product comprising a recombinant protein, wherein the recombinant protein comprises an amino acid sequence that is at least 80% identical to any one of SEQ ID NOs: 54-232. In some embodiments, the food product is substantially animal-free. In some embodiments, the recombinant protein comprises an amino acid sequence that is at least 90% identical to any one of SEQ ID NOs: 54-232. In some embodiments, the recombinant protein comprises an amino acid sequence that is at least 95% identical to any one of SEQ ID NOs: 54-232. In some embodiments, the food product is a cat food product.

In some embodiments, the recombinant protein is a recombinant albumin protein. In some embodiments, the recombinant albumin protein is a recombinant rabbit albumin protein. In some embodiments, the albumin protein is a recombinant duck albumin protein. In some embodiments, the recombinant duck albumin protein is a recombinant mallard duck albumin protein. In some embodiments, the recombinant duck albumin protein is a recombinant Muscovy duck albumin protein. In some embodiments, the recombinant albumin protein is a recombinant cow albumin protein. In some embodiments, the recombinant albumin protein is a recombinant sheep albumin protein. In some embodiments, the recombinant albumin protein is a recombinant pig albumin protein. In some embodiments, the recombinant albumin protein is a recombinant bison albumin protein. In some embodiments, the recombinant albumin protein is a recombinant deer albumin protein. In some embodiments, the recombinant albumin protein is a recombinant chicken albumin protein. In some embodiments, the recombinant albumin protein is a recombinant turkey albumin protein. In some embodiments, the recombinant albumin protein is a recombinant salmon albumin protein. In some embodiments, the recombinant albumin protein is a recombinant cod albumin protein. In some embodiments, the recombinant albumin protein is a recombinant tuna albumin protein.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “figure” and “FIG.” herein), of which:

FIG. 1 shows a Coomassie stained sodium dodecyl sulfate-polyacrylamide gel electrophoresis (SDS-PAGE) gel of an example media from Pichia pastoris cells including a construct for secreted recombinant rabbit albumin or wildtype cells.

FIG. 2 shows a Coomassie stained SDS-PAGE gel of an example media from Pichia pastoris cells including a construct for secreted recombinant rabbit Tropomyosin alpha-1 chain or wildtype cells.

FIG. 3 shows a Coomassie stained SDS-PAGE gel of an example recombinant rabbit albumin.

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

Generation of animal-free protein is a way to reduce global meat production and offset the agricultural and environmental consequences of animal farming. Animal free protein can be made using microorganisms designed to produce meat proteins using recombinant protein expression. Foods, such as pet food (e.g., cat food, dog food, or food for other companion animals) or foods for human consumption can be developed from animal-free meat proteins as an alternative or supplement to traditional meat-based foods.

Using recombinant protein expression methods, microorganisms (e.g., a yeast or bacteria) can be contacted with a DNA expression cassette. This DNA sequence can contain a gene or genes of interest, as well as regulatory sequences which will help in the production of the gene or genes of interest. Regulatory sequences (e.g., UTRs, promoters, signal sequences, etc.) can aid in, for example, transcription or translation efficiency, protein product yield, protein expression, or protein secretion.

Microorganisms can be induced to express and secrete meat proteins of specific animals (e.g., a rabbit protein or a duck protein). Microorganisms can alternatively or additionally be induced to express and secrete specific types of meat proteins (e.g., an albumin protein). Animal-free meat proteins can be purified and formulated into food products (e.g., a pet food or food for human consumption).

### Definitions

Whenever the term “at least,” “greater than,” or “greater than or equal to” precedes the first numerical value in a series of two or more numerical values, the term “at least,” “greater than” or “greater than or equal to” applies to each of the numerical values in that series of numerical values. For example, greater than or equal to 1, 2, or 3 is equivalent to greater than or equal to 1, greater than or equal to 2, or greater than or equal to 3.

Whenever the term “no more than,” “less than,” or “less than or equal to” precedes the first numerical value in a series of two or more numerical values, the term “no more than,” “less than,” or “less than or equal to” applies to each of the numerical values in that series of numerical values. For example, less than or equal to 3, 2, or 1 is equivalent to less than or equal to 3, less than or equal to 2, or less than or equal to 1.

The terms “companion animal” and “pet” are used interchangeably herein. A pet is a domestic or tamed animal that is kept for companionship. A pet can be, but is not limited to, a dog, a cat, a pig, a rat, a mouse, a snake, a turtle, a cow, a sheep, a llama, a donkey, a goat, a horse, a chicken, a parrot, a ferret, a guinea pig, a hamster, a rabbit, a fish, a frog, a toad, a salamander, an iguana, a lizard, a duck, a goose, or an insect.

The term “meat” can be defined as skeletal muscle and its associated tissues, including blood, derived from mammalian, avian, reptilian, amphibian, and aquatic species harvested for consumption. Edible offal comprising organs and non-skeletal muscle tissue can also be considered meat.

The term “animal-free” can be defined as something that is not harvested directly from an animal, living or dead (e.g., meat harvested from a dead animal).

### Food Products

Described herein are food products comprising animal-free proteins. An animal-free protein can be a protein or a fragment or truncation of a protein that is not obtained from an animal (e.g., by harvesting meat from an animal). An animal-free protein can be obtained through in vitro or ex vivo methods such as cell-free protein synthesis (CFPS). Alternatively, an animal-free protein can be obtained through in vivo methods such as genetic engineering of cells for recombinant protein expression. Genetically engineered cells used for recombinant protein expression can be, but are not limited to, microorganism cells (e.g., bacteria, yeast), plant cells, insect cells, or animal cells.

In recombinant protein expression, a cell is contacted with a DNA sequence. The DNA sequence will be incorporated by the cell, and the cell's internal protein expression machinery will transcribe and translate the DNA sequence into a peptide.

A cell used in recombinant protein expression can be a microorganism cell. Microorganisms are microscopic organisms such as bacterium, viruses, or fungi (e.g., yeast). A microorganism cell used in recombinant protein expression can be a bacterial cell. A bacterial cell can be, but is not limited to, a cell from Escherichia coli, Bacillus licheniformis, Bacillus subtilis, or Lactococcus lactis. A microorganism cell used in recombinant protein expression can be a yeast cell. A yeast cell can be, but is not limited to, a cell from Schizosaccharomyces pombe, Saccharomyces cerevisiae, Hansenula polymorpha (also known as Ogataea polymorpha), Pichia farinosa, Pichia anomala, Pichia heedii, Pichia guilliermondii, Pichia kluyveri, Pichia membranifaciens, Pichia norvegensis, Pichia ohmeri, Pichia pastoris (also known as Komagataella phaffii), Yarrowia lipolytica, Pichia methanolica, or Pichia subpelliculosa. A microorganism cell used in recombinant protein expression can be a filamentous fungal cell. A filamentous fungal cell can be, but is not limited to, a cell from Trichoderma reesei (also known as Hypocrea jecorina), Aspergillus niger, Aspergillus oryzae, Aspergillus tubingensis, or Ashbya gossypii (also known as Eremothecium gossypii).

P. pastoris cells can be used in recombinant protein expression methods. P. pastoris cells have robust protein secretion; the ability to grow to high biomass density, allowing higher yields; have a high protein yield with up to 35 g/L yield for secreted proteins; have a low endogenous secretome, allowing for less downstream purification; grow in relatively inexpensive feedstock/media (e.g., glycerol or methanol); have high genetic tractability; have a proven ability to properly express and fold distantly-related eukaryotic (e.g., plant or animal) proteins; have a 90 minute doubling time; have an ability to grow in large-scale fermenters; and have an approximately one-week fermentation time from start to harvest.

A cell used in recombinant protein expression can be chosen because it displays a desired characteristic such as having a rapid doubling time, the ability to properly fold a target recombinant protein; the ability to make appropriate post-translational modifications to a target recombinant protein; the ability to efficiently secrete the target recombinant protein; the ability to be grown cheaply; the ability to be grown on easily accessible media; or genetic tractability. Alternatively, a cell used in recombinant protein expression may not secrete or may not efficiently secrete the target recombinant protein. In an example, cells that do not secrete or do not efficiently secrete the target recombinant protein may be lysed to release the protein. Cell lysis may include mechanical disruption, liquid homogenization, sonication, freeze/thaw cycling, manual grinding, or any combination thereof. In some examples, cells that secrete the target recombinant protein may be further lysed to increase protein yield.

During recombinant protein expression, a cell is contacted with a DNA sequence that can comprise an expression cassette. An expression cassette can be a component of vector DNA which can comprise at least one gene and/or at least one regulatory sequence.

An expression cassette can comprise at least one gene of interest. An expression cassette can comprise at least 1, at least 2, at least 3, at least 4, at least 5, at least 6, at least 7, at least 8, at least 9, at least 10 or more genes of interest. Alternatively, or in addition to, an expression cassette can comprise at least one additional gene other than a gene of interest. An expression cassette can comprise at least 1, at least 2, at least 3, at least 4, at least 5, at least 6, at least 7, at least 8, at least 9, at least 10 or more additional genes other than a gene of interest. An expression cassette can be a linearized plasmid. Alternatively, an expression cassette can be a linear PCR product.

A DNA sequence (e.g., a vector or an expression cassette) can be transfected into a cell using various chemical and physical methods such as electroporation, calcium phosphate exposure, spheroplasting, polyethylene glycol (PEG) exposure, alkali cation exposure, or liposome-based transfection.

A gene of interest can be an animal protein-coding gene. An animal protein-coding gene can encode a full length or a fragment or truncated version of a naturally occurring protein. Alternatively, or in addition to, an animal protein-coding gene can encode a modified version (e.g., a codon-optimized version) of a naturally occurring protein. Alternatively, an animal protein-coding gene can encode a synthetic or novel protein.

An animal protein-coding gene can encode an antibody, a contractile protein, an enzyme, a hormonal protein, a structural protein, a storage protein, or a transport protein. An animal protein-coding gene can be specific to a particular type of tissue such as, but not limited to, connective tissue, epithelial tissue, muscle tissue, or nervous tissue.

An animal protein-coding gene can encode proteins represented in meat or other parts of animals that are eaten. Examples of proteins found in meat can include, but are not limited to: actin, myosin, myosin regulatory protein, albumin, carbonic anhydrase, myoglobin, collagen, elastin, titin, nebulin, sarcalumenin, creatin kinase, phosphoglucomutase, phosphoglycerate mutase, triosephosphate isomerase, alpha-actinin, glycogen phosphorylase, fructose-biphosphate adolase, glyceraldehyde-3-phosphate dehydrogenase, L-lactate dehydrogenase, tropomyosin, myosin-binding proteins, myomesin, beta-enolase, pyruvate kinase, phosphoglycerate kinase, plectin, ATP-dependent 6-phosphofructokinase, malate dehydrogenase, hemoglobin, filamin, aconitate hydratase, parvalbumin, heat shock cognate 71, troponin, calsequestrin, obscurin, glycogen debranching enzyme, elongation factor 1-alpha 2, aspartate aminotransferase, adenylate kinase isoenzyme, glucose-6-phosphate isomerase, desmin, AMP deaminase, and globulin.

An animal protein-coding gene can encode albumin. Albumin is a family of globular proteins, the most common of which are serum albumins. Albumin proteins are water-soluble and are commonly found in blood plasma. Albumin proteins differ from other blood proteins in that they are not glycosylated. Albumin proteins include, but are not limited to: serum albumin, alpha-fetoprotein, vitamin D-binding protein, afamin, ovalbumin and extracellular matrix protein 1.

An animal protein-coding gene can be a gene from a specific animal such as, but not limited to: a canine, a feline, a bird (e.g., a songbird, a pigeon, a chicken, a duck, a goose, a swan, a crow, a dove, a parrot, a turkey), a bovine (e.g., a cow, a bison), a camel, a goat, a pig, a deer, a sheep, a horse, a rodent (e.g., a mouse, a rat, a squirrel, a hamster, a guinea pig, a chipmunk, a gopher, a vole), a fish (e.g., a tuna, a salmon, a trout, a sardine, a tilefish, a halibut, a cod, a flounder), a rabbit, a lizard, a snake, or a frog.

An animal protein can comprise a mouse protein (Table 1). A mouse protein can comprise a mouse muscle protein. A mouse protein can comprise a mouse blood protein. A mouse protein can comprise a mouse albumin protein. A mouse protein can comprise a sequence that has at least or about 70%, at least or about 75%, at least or about 80%, at least or about 90%, at least or about 91%, at least or about 92%, at least or about 93%, at least or about 94%, at least or about 95%, at least or about 96%, at least or about 97% at least or about 98% at least or about 99%, or about 100% sequence identity to any one of SEQ ID NOs: 1-53.

A food product may comprise a fragment or a truncation of a mouse protein. The food product may include a fragment or truncation of any one of SEQ ID Nos: 1-53. A sequence of the fragment or truncation may be identical or substantially identical to a portion of any one of SEQ ID Nos: 1-53. A sequence of the fragment or truncation may be at least about 70%, 80%, 90%, 95%, 98%, or more identical to a portion of a sequence of any one of SEQ ID Nos: 1-53. A length of the fragment or truncation may be at least about 10%, 15%, 20%, 30%, 40%, 50%, 60%, 70%, 80%, 90%, or more of the length of any one of SEQ ID Nos: 1-53. A length of the fragment or truncation may be less than or equal to about 90%, 80%, 70%, 60%, 50%, 40%, 30%, 20%, 15%, 10%, or less than a length of any one of SEQ ID Nos: 1-53.

TABLE 1
Mouse Proteins

| SEQ | | |
|---|---|---|
| ID | | |
| NO | Description | Sequence |
| 1 | Mouse TITIN | MTTQAPMFTQPLQSVVVLEGSTATFEAHVSGSPVPEVSWFRDGQVISTSTLPGVQISF |
| | | SDGRARLMIPAVTKANSGRYSLRATNGSGQATSTAELLVTAETAPPNFSQRLQSMTV |
| | | RQGSQVRLQVRVTGIPTPVVKFYRDGAEIQSSLDFQISQEGDLYSLLIAEAYPEDSGT |
| | | YSVNATNSVGRATSTAELVVQGEEVVPAKKTKTIVSTAQISETRQTRIEKKIEAHFDA |
| | | RSIATVEMVIDGATGQLPHKTPPRIPPKPKSRSPTPPSIAAKAQLARQQSPSPIRHSPSP |
| | | VRHVRAPTPSPVRSVSPAGRISTSPIRSVKSPLLIRKTQTTTMATGPEVPPPWKQEGYV |
| | | ASSTEAEMRETTMTSSTQIRREERWEGRYGVQEQVTISGAAAAAASASVSSSFTAGAI |
| | | TTGTKEVKQETDKSAAVATVVAAVDMARVREPAISAVEQTAQRTTTTAVHIQPAQE |
| | | QARKEAEKTAVTKVVVAADKAKEQELKSRTREVMVTTQEQTHISHEQIRKETEKAF |
| | | VPKVVISATKAKEQETRITGEITTKQEQKRITQETIRQETEEIAASMVVVATAKSTKLE |
| | | AAVGVQEETAIQQDQMHLTHEQMMKETRKTVVPKVIVATPKIKEQDLVSRSREAITT |
| | | KRDQVQITQEKKRKEVETTALSTIAVATAKAKEQETVLRSREAMATRQEHIQVTHG |
| | | QVGVGKKAEAVATVVAAVDQARVREPREPTHVEESHSQQTTLEYGYKEHISTTKVP |
| | | EQPRRPASEPHVVPQAVKPAVIQAPSETHIKTTDQMGMHISSQVKKTTDISTERLVHV |
| | | DKRPRTASPHFTVSKISVPKTEHGYEASIAGSAIATLQKELSATSSTQKITKSVKAPTV |
| | | KPGETRVRAEPTPSPQFPFADMPPPDTYKSQAGIEVKKEVGVSISGSTVREEHFEVLR |
| | | GREAKVTETARVPAPAEVPVTPPTLVSGLKNVTVIEGESVTLECHISGYPSPKVTWYR |
| | | EDYQIESSIDFQITFQGGIARLMIREAFAEDSGRFTCSAVNEAGTVSTSCYLAVQVSEE |
| | | FDKETTLTEKFATEEKRFVESRDVVMTDTSITEEQAGPGEPAAPFFISKPVVQKLVEG |
| | | GSVVFECQIGGNPKPHVYWKKSGVPLTTGYRYKVSYNKQTGECRLVISMTFADDAG |
| | | EYTIVIRNKHGETSASASLLEEADYEALVKTQQEMLYQTQMSTFIQEPKVGEIAPGFA |
| | | YSEYEKEYEKEQALIRKKMAKDTVMVRTFVEDQEFHISSFEERLIKEIEYRIIKTTLEE |
| | | LLEEDGEEKMAVDISESEAIESGFDIRIKNYRILEGMGVTFHCKMSGYPLPKIAWYKD |
| | | GKRIRHGERYQMDFLQDGRASLRIPVVLPEDEGIYTAFASNIKGNAICSGKLYVEPAA |
| | | PFSAPTYMPTPEAVSRIRSVSPRSLSRSPIRMSPAMSPARMSPARMSPARMSPARMSP |
| | | GRRLEETDESQLERLYKPVFVLKPASFKCLEGQTARFDLKVVGRPMPETFWFHNGQ |
| | | QIVNDYTHKVVIKEDGTQSLIIVPASPSDSGEWTVVAQNRAGKSTISVTLTVEAVEHQ |
| | | IKPAFVEKLKNVNIKEGSRLEMKVRATGNPNPDIVWLKNSDIIVPHKYPRIRIEGTRGE |
| | | AALKIDSIISQDSAWYTATAINKAGRDTTRCKVNVEVEFAEPEPERKLIIPRGTYRAKE |
| | | IAAPELEPLHLRYGQEQWEEGDLYDKEKQQKPFFKKKLTSLRLKRFGPAHFECRLTPI |
| | | GDPTMVVEWLHDGKPLEAANRLRMINEFGYCSLDYGAAYSRDSGVITCRATNKYG |
| | | TDHTSATLIVKDEKSLVEESQLPDGKKGLQRIEELERMAHEGALTGVTTDQKEKQKP |
| | | DIVLFPEPVRVLEGETARFRCRVTGYPQPKVNWYLNGQLIRKSKRFRVRYDGIHYLDI |
| | | VDCKSYDTGEVKVTAENPEGVTEHKVKLEIQQREDFRSVLRRAPEPKPEFHVHEPGK |
| | | LQFEVQKVDRPVDTSETKEVVKLKRAERITHEKVSEESEELRSKFKRRTEEGYYEAIT |
| | | AVELKSRKKDESYEELLKKTKDELLHWTKELTEEEKKALAEEGKITIPTFKPERIELSP |
| | | SMEAPKIFERIQSQTVGQGSDAHFRVRVVGKPDPECEWYKNGVKIERSDRIYWYWP |
| | | EDNVCELVIRDVTAEDSASIMVKAINIAGETSSHAFLLVQAKQLITFTQELQDVVAKE |
| | | KDTMATFECETSEPFIKVKWYKDGIEVHAGDKYRMHSDRKVHFLSVLTIDTSDAED |
| | | YSCVLVEDENIKTTAKLIVEGAVVEFVKELQDIEVPESYSGELECIISPENIEGKWYHN |
| | | DVELKSNGKYSITSRRGRQNLTVKDVTKEDQGEYSFVVDGKKTTCKLKMKPRPIAIL |
| | | QGLSDQKVCEGDIVQLEVKVSLENVEGVWMKDGQEVQHSDRVHIVIDKQSHMLLIE |
| | | DMTKEDAGNYSFTIPALGLSTSGNVSVYSVDVITPLKDVNVIEGTKAVLECKVSVPD |
| | | VTSVKWYLNDEQIKPDDRVQSIVKGTKQRLVINRTHASDEGPYKLMVGRVETSCNL |
| | | SVEKIKIIRGLRDLTCTETQNVVFEVELSHSGIDVVWNFKGKEIKPSSKYKIEAHGKIY |
| | | KLTVLNMMKDDEGEYAFYAGENTTSGKLTVAGGAISTPLTDQTVAESQEAVFECEV |
| | | ANPESEGEWLKDGKHLALSNNFRGESDGHKRRLVIAAAKLDDAGEYTYKVATSKTS |
| | | AKLKVEAVKIKKTLRNLTVTETQDAVFSVELTHPDVKGVQWIKNGVVLDSNDKYEI |
| | | SVKGTLYSLKIKNCAMADESVYGFKLGRLGASARLHVETVKIIKKPKDVTALENATV |
| | | TFEVSVSHDTVPVKWFHKNVEIKPSDKHRLVSERKVHKLMLQSISPSDAGEYTAMV |
| | | GQLECKAKLFVETLHITKTMKSIEVPETKAASFECEVSHFNVPSMWLKNGVEIEMSE |
| | | KFKIVVQGKLHQLIIMNTSTEDSAEYTFVCGNDQVSATLTVTPIMITSMLKDINAEEK |
| | | DTITFEVTVNYEGISYKWLKNGVEIKSTDRCQMRTKKLTHSLNIRNVHFGDAADYTF |
| | | VAGKATSTATLYVEARHIEFRKHIKDIKVLEKKRAMFECEVSEPDITVQWMKDGQEL |
| | | QIADRIKIQKEKYVHRLLIPSTRMSDAGKYTVVAGGNMSTANLFVEGRDVRIRSIKKE |
| | | VQVIEKQRAVVEFEVNEDDVDAHWYKDGIEINFQVQERHQYVVERRIHRMFISETRH |
| | | SDAGEYTFVAGRNRSSVTLYVNAPEPPQVLQELQPVTVQSGKPARFCAVISGRPQPKI |
| | | SWYKEEQLLSTGFKCKFLHDGQEYTLLLIEAFPEDAAVYTCEAKNDYGVATTSASLS |
| | | VEVPEVVSPDQEMPVYPPAIVTPLQDTVTSEGRPARFQCQVSGTDLKVSWYCKDKKI |
| | | KPSRFFRMTQFEDTYQLEIAEAYPEDEGTYAFVANNAVGQVSSTATLRLEAPESILHE |
| | | RIGQQIEMEMKEIASLLSAEEDFQTYSSDLRLPNANETLELLSEPPARSTQFDSRQEGA |
| | | APVFIREISDVEISVEDVAKLSVTVTGCPKPKIQWFFNGMLLTPSADYKFVFDGDTHS |
| | | LIILFTRFQDEGEYTCLASNEYGKAVCSAHLRISPRGERSTEMESGEKKALEKPKGPCP |
| | | PYFFKELKPVHCGPGIPAVFEYSVHGEPAPTVLWFKEDMPLYTSVCYTIIHSPDGSGT |
| | | FIVNDPQRGDSGLYLCKAQNLWGESTCAAELLVLPEDTDVPDASCKEESTLGVPGDF |
| | | LETSARGPLVQGVDSRQEITAFAEGTISKAALIAEETLQLSYERSVDDSEVGTGVTIGA |
| | | QKLPPVVLSTPQGTGELPSIDGAVHTQPGRGPPPTLNLQAVQAQTTLPKEATLQFEEP |
| | | EGVFPGASSAAQVSPVTIKPLITLTAEPKGNYPQSSTAAPDHALLSSVAAETLQLGEK |
| | | KIPEVDKAQRALLLSQSLAEGCVESLEVPDVAVSNMRSEPQVPFQHTCTEGKILMAS |
| | | ADTLKSTGQDVALRTEEGKSLSFPLALEEKQVLLKEEQSEVVAVPTSQTSKSEKEPEA |
| | | IKGVKEVREQELLSKETLFPSMPEEQRLHLKTQVRRALQAAVAREQANLFSEWLRNI |
| | | DKVEVTAVNFTQEPKRILCTYLITSVSSLTEELTVTIEDIDPQMANLETGLKDALCSIV |
| | | CEERNILMAEDPRIHEEDKIDVQGGRDHLSDAQKVETVIEAEADSKYLVSKEEVSWS |
| | | KVESQLKDGDTNEVPQAETLKLAEESGTQKTSTEMSQEEAEGTLADLCPAVLKHLV |
| | | DTISEEGDTVHLTSSISNAKEVHWYFKGNLVPSDGKFKCLKEQNAYTLVIEAVKTED |
| | | EGEYVCEASNDSGKAKTSAKLTVGERAAPVIKRRIEPLEVALGHLAKFTCEIQGAPN |
| | | VRFQWFKAGREIYESDKCSIRSSNYVSSLEILRTQVVDCGEYTCKASNEYGSVSCTAT |
| | | LTVTEAYPPTFLSRPKALTTFVGKAAKFLCTVSGTPVIEIIWQKDGAALSPSPDCRVTD |
| | | ADNKHSLELSNLTVQDRGIYSCKASNKFGADICQAELTIIDKPHFIKELEAVQSAINKK |
| | | IHLECQVDEDRKVTITWSKDGQKLPAGKDYKIYFEDKIASLEIPLAKLKDSGTYTCTA |
| | | SNEAGSSSSSAAVAVREPPSFVKKVDPSYLMLPGESARLHCKLKGSPVIQVTWFKNN |
| | | KELSESNTVRMSFVNSEAILDITDVKVDDSGTYSCEATNDVGSDSCSTEVVIKEPPSFI |
| | | KTLEPADIVRGANALLQCEIAGTGPFEVNWFKDKKQIRSSKKYRLFTQKTFVYLEISS |
| | | FNSADVGDYECVVANEVGKCGCVATHLLKEPPTFVKKVDDFTALAGQTVTLQAAV |
| | | RGSEPISVMWMKGQEVIKEDGKIKMSFSNGVAVLTIPDVQISLGGKYTCLAENEAGS |
| | | QTSVGELIVKEPAKIIERAELIQVTAGDPATLEYTVSGTPELKPKWYKDGRPLVASKK |
| | | YRISFKNNIAQLKFYSAELHDSGQYTFEISNEVGSSSCETTFTVLDRDIAPLFTKPLRN |
| | | VDSVVGGACRLDCKIAGSLPMRVSWFKDGKELTASDRYQIAFVEGTASLEISRVDM |
| | | NDAGNFTCRATNSVGSKDSSGALIVQEPPSFVTKPGSRDVLPGSAVCLKSAFQGSAPL |
| | | TIKWFKGDKELVSGGSCYITKETSESSLELYAVKTSDSGTYTCKVSNVAGSVECSAD |
| | | LFVKEPATFIEKLEPSQLLKKGDGTQLACKVTGTPPIKITWFANDRELRESSKHKMSF |
| | | AESTAVLRLTDVAIEDSGEYMCEAQNEAGSDHCTGIVIVKESPYFTKEFKSIEVLKEY |
| | | DVMLLAEVAGTPPFEITWFKDNTTLRSGRKYKTFLQDQLVSLQVLKFVAADAGEYQ |
| | | CRVTNEVGSSTCSARVTLREPPSFIKKIEATSSLRGGTAAFQATLKGSLPITVTWLKDN |
| | | DEITEDDNIRMTFENNVASLYLSGIEVKHDGKYVCQAKNDAGIQRCSALLSVKEPATI |
| | | MEEAVSIDVTQGDPATLQVKFSGTKEISAKWFKDGQELTLGPKYKISVTDTVSILKIIS |
| | | TEKKDSGEYTFEVQNDVGRSSCKASINVLDLIIPPSFTKKLRKMDSIKGSFIDLECIVA |
| | | GSHPISIQWFKDDQEISASDKHKFSFHDNTAFLEISQLEGTDSGTYTCSATNKAGHSQ |
| | | CSGHLTVKEPPYFVEKPQSQDVNPGTRVQLKALVGGTAPMTIKWFKDNKELHPGAA |
| | | RSVWKDDTSTILELFSAKAADSGTYICQLSNDVGTTSSKATIFVKEPPQFIKKPSPVLV |
| | | LRNGQSTTFECQVTGTPEIRVSWYLDGNEITDLRKYGISFVDGLATFQISNARVENSG |
| | | TYVCEARNDAGTASCSIELKVKEPPIFIRELEPVEVVKDSDVELECEVMGTTPFEVTW |
| | | LKNNKEIRSGKKYTMSEKMSVFYLHITKCDPSDVGEYQCIIANEGGSCACSARVALK |
| | | EPPSFIKKIENVTTVLKSSATFQSTVAGSPPISITWLKDDQILEENDNVHISFEDSVATL |
| | | QVRSVDNGHSGRYTCQAKNESGIERCYAFLLVQEPAQIIEKAKSVDVTEKDPVTLEC |
| | | VVAGTPELKVKWLKDGKQIVPSRYFSMSFENNVASFRIQSVMKQDSGQYTFKVEND |
| | | FGSSSCDAYLRVLDQDIPPSFTKKLTKMDKVLGSSIHMECKVSGSLPISAQWFKDGKE |
| | | ISTSAKYRLVCHENTVSLEVSNLELEDTANYTCKVSNVAGDNACSGILTVKEPPSFLV |
| | | KPERQQAIPDSTVEFKAVLKGTPPFKIKWLKDDVELVSGPKCFIGLEGSTSFLNLYSV |
| | | DSSKTGQYTCQVTNDVGSDSCTTMLLVTEPPKFVKKLEASKIIKAGDSARLECKITGS |
| | | PEIQVVWYRNEHELTASDKYQMTFIDSVAVIQMNSLGTEDSGDFICEAQNPAGSTSC |
| | | STKVIVKEPPVFSSFPPIVETLKNTEVSLECELSGTPPFEVVWYKDKRQLRSSKKYKV |
| | | ASKNFHASIHILNVESTDIGEYHCKAQNEVGSDACVCAVKLKEPPKFISKLNSLTVVA |
| | | GEPAELQASIEGAQPISVQWLKEKEEVIRESENIRISFVNNVATLQFAKVEPANAGKYI |
| | | CQVKNDGGVRENMATLTVLEPAVIIEKAGSMTVTVGETCALECKVAGTPELSVEWY |
| | | KDGKLLTSSQKHKFSFYNKISSLKILSVEKEDAGTYTFQVQNNVGKSSCTAVVDVSD |
| | | RMVPPSFTRRLKDTGGVLGTSCILECKVAGSSPISIAWFHEKTKIVSGAKYQTTFSDN |
| | | VCTLQLNSLDSSDMGSYTCVAANVAGSDECRALLTVQEPPSFVKEPEPLEVLPGKNI |
| | | TFTSVIRGTPPFKVGWFRGARELVKGNRCNIYFEDTVAELELFNIDISQSGEYTCVVS |
| | | NNAGQASCTTRLFVKEPATFVKKLSDHSVEPGKSIILEGTYTGTLPISVTWKKDGVSI |
| | | TPSERCNIVTTEKTCILEILSSTKGDAGHYSCEIENEAGRDACDALVSTLEPPYFVTELE |
| | | PLEASVGDSVSLQCQVAGTPEITVSWFKGDTKLRSTPEYRTYFTNNVATLVFNKVSIN |
| | | DSGEYTCMAENSIGTAASKTIFRIQERQLPPSFARQLKDIEQTVGLPVTLTCRLNGSAP |
| | | IQVCWYRDGVLLRDDENLQMSFVDNVATLKILQTDLSHSGQYSCSASNPLGTASSTA |
| | | RLTAREPKKSPFFDIKPVSIDVIAGESADFECHVTGAQPMRVTWSKDNKEIRPGGNYT |
| | | ITCVGNTPHLRILKVGKGDSGQYTCQATNDVGKDMCSAQLSVKEPPKFIKKLDASKV |
| | | AKQGESIQLECKISGSPEIKVVWFRNDSELHESWKYNMSFVNSVALLTINEASAEDTG |
| | | DYICEAHNGVGDASCSTALKVKAPPVFTQKPPPVGALKGSDVILQCEISGTPPFEVVW |
| | | VKDRKQVRSSKKFKITSKNFDTSLHIFNLEAPDIGEYHCKATNEVGSDTCACTVKFKE |
| | | PPRFVKKLSDASTLIGDPVELQAVVEGFQPISVVWLKDKGEVIRESENVRISFVDNIAT |
| | | LQLGSPEASQSGKYVCQIKNDAGMRECSAVLTVLEPATIVEKPEPMTVTTGNPFTLE |
| | | CVVAGTPELSAKWFKDGRELSSGSRHHITFVRNLASLKIPSAEMNDKGLYTFEVENR |
| | | VGKSSCTVSVHVSDRVVPPSFVRRLKDTSATLGASVVLECRVSGSAPISVGWFLDGN |
| | | EIISSPKCQSSFADNVCTLTLSSLEPSDTGAYTCVAANVAGQDESSAVLTVQEPPSFEQ |
| | | TPDSVEVLPGMSLTFTSVIRGTPPFKVKWFKGSRELVSGEACTISLEDFVTELELLEVE |
| | | PGQSGDYSCLVTNDAGSASCTTHLFVKEPATFVKRLADTSVETGSPIVLEATYSGTPP |
| | | ISVSWMKNEYPLSQSPNCGITTTEKSSILEILESTIEDYAQYACLIENEAGQDICEALVS |
| | | VLEPPYFIEPLEHVEAAIGEPITLQCKVDGTPEIRISWYKEHTKLRSAPAYKMQFKNN |
| | | VASLVINKVDHSDVGEYTCKAENSVGAVASSAVLVIKERKLPPSFARKLKDVHETLG |
| | | FPVAFECRINGSEPLQVSWYKDGELLKDDANLQMSFVHHVATLQILQTDQSHVGQY |
| | | NCSASNPLGTASSSAKLILSEHEVPPFFDLKPVSVDLALGESGSFKCHVTGTAPIKITW |
| | | AKDNREIRPGGNYKMTLVENTATLTVLKVAKGDAGQYTCYASNVAGKDSCSAQLG |
| | | VQEPPRFIKKLDQSRIVKQDEYTRYECKIGGSPEIKVLWYKDEVEIQESSKFRMSFEDS |
| | | VAILEMHSLSVEDSGDYTCEARNAAGSASSSTSLKVKEPPVFRKKPFPVETLKGADV |
| | | HLECELQGTPPFQVSWHKDKRELRSGKKYKIMSENLLTSIHILNVDTADIGEYQCKA |
| | | TNDVGSDTCVGSVTMKAPPQFVKKLTDISTIIGKEVQLQTTIEGAEPISVAWFKDKGE |
| | | IVRESDNIWISYSENIATLQFSRAEPANAGKYTCQIKNDAGMQECYATLSVLEPAAIV |
| | | EKPESIKVTTGDTCTLECTVSGTPELSTKWFKDGKELTSDNKYKISFFNKVSGLKIINV |
| | | VPGDSGVYSFEVQNPVGKDSCKVSIQVSDRIIPPSFTRKLKETNGLSGSSVVMECKVY |
| | | GSPPISVLWFHDGNEISSGRKYQTTLTDNTCALTVNMLEEADAGDYTCIATNVAGSD |
| | | ECSAPLTVREPPSFVQKPDPMDVLTGSNVTFTSIVKGSPPFTVSWFKGSTELVPGARC |
| | | NVSLQDSVGELELFDVDTSQSGEYTCIVSNEAGRASCTTRLFVKAPAIFVKRLNDYSI |
| | | EKGKPLILEGTFSGTPPISVTWKKNGINVIASQRCNITTTEKSAILEILSSTVEDSGQYN |
| | | CYIENASGKDSCSAQILILEPPYFVKQLEPVKVTVGDSASLQCQLAGTPEIGVSWYKG |
| | | DTKLRPTATCKMHFKNNVATLVFTQVDSSDSGEYICRAENSVGEVSSSTFLTVQEQK |
| | | LPPSFSRQLRDVQETVGLPVVFECAVSGSEPISVSWYKDGKPLKDSPNIQTSFLDNIAT |
| | | LNIFKTDRSLSGQYSCTATNPIGSASSGAKLILTEGKNPPFFDIPLAPMDAVVGESADL |
| | | ECHVTGTQPIKVTWAKDNREIRSGGNYQISYLENSAHLTIVKVDKGDSGQYTCYAV |
| | | NEVGKDSCTAQLNIKERLIPPSFTKKLSETVEETEGNSFKLEGRVAGSQPITVAWYKN |
| | | NVEIHPTSNCEIMFKNNALLLQVKRASMADAGLYTCKATNDAGSALCTSSIVIKEPK |
| | | KPPVFDQHLAPVTASEGDSVQLSCHVQGSEPIRIQWLKAGREVKPSDRCSFSFASGTA |
| | | MLELKETAKADSGDYVCKASNVAGSDTSKCKVTIKEKPAAAPAAKKAAVDGKLFF |
| | | VSEPQSIRVVEKTTATFIAKVGGDPIPNVKWTKGKWRQLNQGGRILIHQKGDEAKLEI |
| | | RDTTKTDSGLYRCVAFNKHGEIESNVNLQVDERKKQEKIEGDLRAMLKKTPALKKG |
| | | SGEEEEIDIMELLKNVDPKEYEKYARMYGITDFRGLLQAFELLKQSQEEETHRLEIEE |
| | | LEKSERDEKEFEELVAFIQQRLTQTEPVTLIKDIENQTVLKDNDAIFEIDIKINYPEIKLS |
| | | WYKGTEKLEPSNKYEISIDGDRHTLRVKNCQPKDQGNYRLVCGPHIASAKLTVIEPA |
| | | WERHLQDVTLKEGQTCTMTCQFSVPNVKSEWFRNGRVLKPQGRVKTEVEHKVHKL |
| | | TIADVRAEDQGQYTCKHEDLETSAELRIEAEPIQFTKRIQNIVVSEHQSATFECEVSFD |
| | | DAIVTWYKGPTELTESQKYNFRNDGRCHYMTIHNVTPDDEGVYSVIARLEPRGEARS |
| | | TAELYLTTKEIKLEMKPPDIPDSRVPIPTMPIRAVPPEEIPPAVAPSIPLLLPLPEEKKPP |
| | | AKRIEVTKKGVKKDTKKVVTKPKEEAPPPPVAKKPPPPTPMIPAKASEIIDVSSKAEE |
| | | VKITTITRKKEVHKEKEAVYEREEAVYEKKVHIEPWEEPYEELETEPYTEPYEEPYYE |
| | | EPDEDYEEIKVEAKKQVHEEWEEDFEEGQEYYEREEGYDEGEEEWEEIYHEREIIQV |
| | | QKEVHEELHEKKIPAKVPEKKVPPPKVVKKPVVEKVEKTTRRMEEEKVQVIKVPEVS |
| | | KKIVPQKPSRTPVQEEIIEVKVPAVHTKKMVISEEKMFFASHTEEEVSVSVPEVQKKT |
| | | VTEEKIHVAVSKKIEPPPKVPEPPKKPVPEEVVPVPIPKKVEPPAAKVPEAPKKPVPEE |
| | | KKPVPIPKKKEPAAPPQVPEAPKKPAPEEKIPVPVTKKKEAPPAKVPEVQKKVVTEEK |
| | | IAIITQREESPPPAVPEIPKKKVPEEKRPVPRKEEVPPPKVPVPPKKAVPEAVVPAPIPK |
| | | KAPPRAEVSKKTVVEEKRFAAEEKLSVAVPQRVELMRHEEEEWTYSEEEERVSVSV |
| | | YREEERDEEEAEITEYEVLEEPEEYVVEEKMHFISKKVEVEPAKVPEKKIIPKPKVPAK |
| | | IEEPPPTKVPEPPKKIVPEKKVPAPAPKKVPPAKAPEESKRPVPEKRAPAEEVGIEEPPP |
| | | TKVAERHMKITQEEKVLVAVTKKEAPPRARVPEEPKKVAPEERFPKLKPRREEEPPA |
| | | KVTEVRKRAVKEEKVSIEVPKREPRPTKEVTVTEEKKWSYTREEETVSEHREEEYED |
| | | YEDYEEYKEFEEYEPTEEYDQYDEYAEREVEHYEEHEEYVTEPKKPVPVKPAQEPVP |
| | | AKPKAPPPKVLKKAVPEEKAPLPIQKKLKPLPPKAPEEPKKVVEEKIQISITKREKQQV |
| | | TEPVAKVPMKPKRVVPEAKIPAPTKEVAVPVRVPGVPKKRELEEVVVFKEEVEAHEE |
| | | YIVEEEEEYVHEEEYVHKEEYVHEEEYVHKEEYIHEEEEHLHEEEETIAEEEVVPVAP |
| | | VKVPVVPKKPVPEEKKPVPVPKKKEAPPAKVPEIPKKPEEKVPVPIPKKEKAPPAKVP |
| | | EVPKKPVPEEKPPVPVPKKVEPPPAKVPEVPKKPVPEKKVPAPTPKKVEAPPAKVPEV |
| | | PKKPIPEEKKPTALLKKMEAPPPKAPKKREVVPVPVALPREEEEEEVPFEEVPEEEILP |
| | | EEEVPSEEEAPPEEVPPEEEEVLPEEEEVLPEEEEVLPEEEEVQPEEEALPEIKPKVPKP |
| | | APVPKKTVPEKKVPVPVPKKVEPPPPPKVPEIKKKVPEKKVVVPKKEEAPPTKVPEVS |
| | | KKVEERRIIPPKEEEVPPAEVYEEAEEPTPEEIPEEPPSIEEEEIVEEEEEEEEVLPPRAPE |
| | | VVKKAVPEAPTPVPKKAEAPPAKVPKKIPEEKVPVPVQKKEAPPAKVPEKKKIPEKK |
| | | VPVPKKEAVPPAKGKAVFEEKISVAYQQEELVQERIELELVEAKVEEAFEEEEFHEVQ |
| | | EYFEEEEFHEVEEFIRVEERRFQEEHKVEEVHRVIEFLEAEEVEVYEKPKIPPKKGPEV |
| | | SEKVIPPKKPPTKVIPRKEPPAKVPEVTKKTVVEEKIRAPEEPKVPAPKAPEVPKKITPE |
| | | EKVREAVPKKPEVPPPKVPEVPKKIIQEEKLPVVLPEDTEIYMYEASEETVIEEEHVTL |
| | | PQKARLKVAKVPAPPQTVVTEEKTYVTIRKTRETLALKESETTREAFPELKSYKAVPE |
| | | IPEPPSPEDLEIIEDVLPEKRPPASKRRKTQLPTAPEAPREMPPEMNTFEEISVEPEMLPT |
| | | QVLDTYQEATVEKKTLRISRKKPELPSDEEVPEAPREVVAKKKVLPPQVPEVVPVKV |
| | | PGAPKEVVSERKSLEEPPKKPAVRPVTVPEEPKEVIPEKKVSLVPPKKPAAPPVTVPE |
| | | APEEVFSEDEETLAPPQEPEAPPAKVPEAPKEVVPEKKVSVVPPKKPEAPPAKVPEAP |
| | | KEAAPEKKVPVAPKKKPEAPPVKVPEAPKKVVPEKKLPVAAPKKPEAPAAEVPEVP |
| | | KTAVPQKKIPEAIPPKPESPPLEVPEVPPKEVTPEKKVPAAPPTKPEIPPPKVPEAPQAA |
| | | VVEEKTPEALPKKAEAAPVPVPQVQETVPEKTRPVGPPKKPEATTVPVPKVQKTIPEK |
| | | TRPEAPPKRPEARTVPETVPEKTRPMAPPKKPEATTLPVPEVQETVPEKTRPVGPPKK |
| | | PEATTVPVPEVQETIPEKTRPPKKPEATPVPVPEVQETVPEKTRPVGPPKKPEATTVSV |
| | | PEVQETIPEKTRPAAPPKKPEATAVPETIPEKTRPEAPPKRPEATTVPVPEADQAVVPE |
| | | KKVPRVPPKKVEAPPITVPEEPKEVIPEKKVSLVPPKKPAAPPVTVPEAPEEVFSEDEE |
| | | TLAPPQEPEAPPAKVPEAPKEVVPEKKVSVVPPKKPEAPPAKVPEAPKEAAPEKKVP |
| | | VAPKKKPEAPPVKVPEAPKKVVPEKKLPVAAPKKPEAPAAEVPEVPKAAVPQKKIPE |
| | | AIPPKPESPPPEVYEEPEEEIVPEEPPEEAVEEPVPAPPPKVTEPPKKPVPEKKAPPAVV |
| | | KKPEPPPAKVPEVPKEAPPEKKVPPKKPEAPPAKVPEVPKEVVTEKKVAVPKKPEVPP |
| | | AKVPEVPKKPVIEEKPAIPVVEKVASPPAEVYEEPEEVTAEEEEPAPAVEEEEYEAPPP |
| | | PAPVPEEPKKVVPEKKFPVIKKPEAPPPKVPEVPKKAVPVKKVPVVKKPEPPEAEVPE |
| | | VPKKLVPVKKEPVPVTKKTEVLPEKVPEAPKKITPEKKESVPVPEEPEAPPASVEETPE |
| | | ETIYEEKATITIGRKETPPVEEREIEKFIQPEEPELEPEPEEIPVQEPEPEKKVIEKPKLKP |
| | | RPPARPPSPPKEDVKEKMFQLKAVSKKKVPEKPEVVEKVEPAPLKVPTAEKKVRKLL |
| | | PEPKPQPKEEVVLKSVLRKKPEEEEPKVEPKKVEKAKKPEEPQPPPKAVEVEAPPEPT |
| | | PKERKVPEPAKVPEIKPAIPLPGPEPKPKPEPEVKTMKAPPIEPAPTPIAAPVTAPVVGK |
| | | KAEAKPKDEAAKPKGPIKGVAKKTPSPIEAERKKLRPGSGGEKPPDEAPFTYQLKAV |
| | | PLKFVKEIKDIVLTEAESVGSSAIFECLVSPSTAITTWMKDGSNIRESPKHRFIADGKD |
| | | RKLHIIDVQLSDAGEYTCVLRLGNKEKTSTAKLIVEELPVRFVKTLEEEVTVVKGQPL |
| | | YLSCELNKERDVVWRKDGKIVVEKPGRIVPGVIGLMRALTINDADDTDAGTYTVTV |
| | | ENANNLECSSCVKVVEIIREWLVKPIRDQHVKPKGTAVFTCDIAKDTPNIKWFKGYD |
| | | EIPLEPNDKTEILKEGNHLFLKVKNAMPEDIDEYAVEIEGKRYPAKLTLGEREVELLK |
| | | PIEDVTIYEKESASFDAEISEEDIPGEWKLKGELLRPSPTCEIKAEGGKRFLTLHKVKL |
| | | DQAGEVLYQACNAITTAILTVKEIELDFAVPLKDVTVPEKRQARFECVLTREANVIW |
| | | SKGPDIIKASDKFDIIADGKKHILVINDSQFDDEGVYTAEVEGKKTSAQLFVTGIRLKF |
| | | ISPLEDQTVKEGQTATFVCELSHEKMHVVWFKNDVKLHTTRTVLMSSEGKTYKLEIR |
| | | ETTLDDISQIKAQVKNLSSTANLKVLEADPYFTVKLHDKTGVEKDEIILKCEVSKDVP |
| | | VKWFKDGEEIVPSPKHSVKTDGLRRILKIKKAELKDKGEYVCDCGTDTTKANVTVE |
| | | ARLIKVEKPLYGVEVFVGETARFEIELSEPDVHGQWKLKGEPLTASPDCEIIEDGKKH |
| | | VLVLYNCQLDMTGEISFQAANAKSAANLKVKELPLIFITPLSDVKVFEKDEAKFECEV |
| | | SREPKTFRWLKGTQEITGDDRFELIKDGTRHSLVIKSAAFEDEAKYMFEAEDKRTSG |
| | | KLIIEGIRLKFLTPLKDVTAKERENAVFTVELSHDNIPVSWFKNDQRLHTSKRVSMHD |
| | | EGKTHSITFKDLSIDDTSQIRVEAMGISSEAKLTVLEGDPYFTGKLQDYTGVEKDEVIL |
| | | QCEISKADAPVKWFKDGKEIKPSKNVVIKADGKKRMLILKKALKSDIGQYTCDCGTD |
| | | QTSGKLDIEDREIKLVRPLYSVEVMETETARFETEISEDDIHANWKLKGEALLQTPEC |
| | | EIKEEGKIHVLILHNCRLDQTGGVDFQAANVKSSAHLRVKPRVIGLLRPLKDVTVTA |
| | | GETATFDCELSYEDIPVEWYLKGKKLEPNDKVVTRSEGRVHTLTLRDVKLEDAGEV |
| | | QLTAKDFKTQANLFVKEPPVEFTKPLEDQTVEEEATAVLECEVSRENAKVKWFKNG |
| | | TEILKSKKYEIVADGRVRKLIIHGCTPEDIKTYTCDAKDFKTSCNLNVVPPHVEFLRPL |
| | | TDLQVKEKETARFECEISKENEKVQWFKDGAEIKKGKKYDIISKGAVRILVINKCLLN |
| | | DEAEYSCEVRTARTSGMLTVLEEEAVFTKNLANLEVSEGDTIKLVCEVSKPGAEVIW |
| | | YKGDEEIIETGRFEILTDGRKRILIIQNAQLEDAGSYNCRLPSSRTDSKVKVHELAAEFI |
| | | SKPQNLEILEGEKAEFVCTISKESFEVQWKRDDQTLESGDKYDIIADGKKRVLVVKD |
| | | ATLQDMGTYVVMVGAARAAAHLTVIEKLRIIVPLKDTKVKEQQEVVENCEVNTEGA |
| | | KAKWFRNEEAIFDSSKYIILQKDLVYTLRIRDARLDDQANFNVSLTNHRGENVKSAA |
| | | NLIVEEEDLRIVEPLKDIETMEKKSVTFWCKVNRLNVTLKWTKNGEEVAFDNRISYRI |
| | | DKYKHSLIIKDCGFPDEGEYVVTAGQDKSVAELLIIEAPTEFVEHLEDQTVTEFDDAV |
| | | FSCQLSREKANVKWYRNGREIKEGKKYKFEKDGSIHRLIIKDCRLEDECEYACGVED |
| | | RKSRARLFVEEIPVEIIRPPQDILEAPGADVIFLAELNKDKVEVQWLRNNMIVVQGDK |
| | | HQMMSEGKIHRLQICDIKPRDQGEYRFIAKDKEARAKLELAAAPKIKTADQDLVVDA |
| | | GQPLTMVVPYDAYPKAEAEWFKENEPLSTKTVDTTAEQTSFRISEAKKDDKGRYKIV |
| | | LQNKHGKAEGFINLQVIDVPGPVRNLEVTETFDGEVSLAWEEPLTDGGSKIIGYVVER |
| | | RDIKRKTWVLVTDRADSCEFTVTGLQKGGVEYLFRVSARNRVGTGEPVETDSPVEA |
| | | RSKYDVPGPPLNVTITDVNRFGVSLTWEPPEYDGGAEITNYVIELRDKTSIRWDTAM |
| | | TVRAEDLSATVTDVVEGQEYSFRVRAQNRIGVGKPSAATPFVKVADPIERPSPPVNL |
| | | NASEQTQSSVQLTWEPPLKDGGSPILGYIIERREEGKDNWIRCNMKPVPELTYKVTGL |
| | | QKGNKYLYRVSAENAAGVSDPSEILGPLTADDAFVEPTMDLSAFKDGLEVIVPNPIKI |
| | | LVPSTGYPRPKATWTFGDQVLEEGDRVKMKTISAYAELVISPSERTDKGIYTLTLENP |
| | | VKSISGEINVNVIAPPSAPKELKFSDITKDSVHLTWEPPDDDGGSPLTGYVVEKRDMS |
| | | RKTWTKVMDFVTDLEFTVPDLVQGKEYLFKVCARNKCGPGEPAYTDEPVNMSAPA |
| | | TVPDPPENVKWRDRTANSIFLTWDPPKNDGGSRIKGYIVEKCPRGSDKWVACGEPVP |
| | | DTKMEVTGLEEGKWYAYRVKALNRQGASKPSKPTEEIQAVDTQEAPEIFLDVKLLA |
| | | GITVKAGTKIELPATVTGKPEPKITWTKADTLLKPDQRITIENVPKKSTVTITDSKRSD |
| | | TGTYIIEAVNVCGRATAVVEVNVLDKPGPPAAFDITDVTNESCLLTWNPPRDDGGSK |
| | | ITNYVVERKATDSDVWHKLSSTVKDTNFKATKLTPNKEYIFRVAAENMYGVGEPVQ |
| | | AAPIIAKYQFDPPGPPTRLEPSDITKDAVTLTWCEPDDDGGSPITGYWVERLDPDTDK |
| | | WVRCNKMPVKDTTYRVKGLTNKKKYRFRVLAENLAGPGKPSRSTEPILIKDPIDPPW |
| | | PPGKPTVKDIGKTSLVLNWTKPEHDGGAKIESYVIEMLKTGTDEWVRVAEGVPTTEH |
| | | LLTGLMEGQEYSFRVRAVNKAGESEPSEPSDPVLCREKLYPPSPPRWLEVINITKNTA |
| | | DLKWTVPEKDGGSPITNYIVEKRDVRRKGWQTVDTTVKDTKCTVTPLTEGSLYVFR |
| | | VAAENAIGQSDYTEIGDSVLAKDTFTTPGPPYALTVVDVTKRHVDLKWEPPKNDGG |
| | | RPIQRYIIEKKEKLGTRWVKAGKTSGPDCNFRVTDVIEGTEVQFQVRAENEAGVGHP |
| | | SEPTEILSIEDPTSPPSPPLDLHVTDAGRKHIAIAWKPPEKNGGSPIIGYHVEMCPVGTE |
| | | KWMRVNSRPIKDLKFKVEEGIVPDKEYVLRVRAVNAVGVSEPSEISENVVAKDPDC |
| | | KPTIDLETHDIVVIEGEKLNIPVPFRAVPVPTVSWHKDGKEVKASDRLTMKNDHISAH |
| | | LEVPKSVHADAGVYTITLENKLGSATASINVKVIGLPGPCKDIKASDITKSSCKLTWE |
| | | PPEFDGGSPILHYVLERREAGRRTYIPVMSGENKLSWTVKDLIPNGEYFFRVKAVNKI |
| | | GGGEYIELKNPVIAQDPKQPPDPPVDVEVHNPTAKAMTITWKPPLYDGGSKIMGYIIE |
| | | KITKGEDRWKRCNEHLVPVLTYTAKGLEEGKEYQFRVRAENAAGIGEPSRATPPTKA |
| | | VDPIDAPKVILRTSLEVKRGDEIALDATISGSPYPTITWIKDENVIVPEEIKKRAAPPVR |
| | | RKKGEAEEEEPFSLPLTERLSINNSKQGESQLRIRDSLRPDHGQYMIKVENDHGVAKA |
| | | PCSVSVLDTPGPPINFVFEDIRKDSVLCKWEPPLDDGGSEIINYTLEKKDKTKPDSDWI |
| | | VITSTLRNCKYSVTKLIEGKEYLFRVRAENRFGPGPPCVSKPLLAKDPFEPPDAPDKPI |
| | | VEDVTSNSMLVKWNEPKDNGSPILGYWLEKREVNSTHWSRVNKTLLSSLKTKVDGL |
| | | LEGLTYVFRVCAENAAGPGKFSPPSDPKTARDPISPPGPPVPRVADTSSTTIELEWEPP |
| | | AFNGGGEIMGYFVDKQLVGTNEWSRCTEKMIKVRQFTVKEIREGADYKLRVSAVNA |
| | | AGEGPPGETEPVTVAEPQEPPTVELDVSVKGGIQIMAGKTLRIPAEVTGRPVPTKVWT |
| | | IEEGELDKERVIIENVGTKSELIIKNALRKDHGRYVITATNSCGSKFAAVRVEVFDVPG |
| | | PVLDLKPVVTNRKMCLLNWSDPADDGGSDITGFIIERKDAKMHTWRQPIETERSKCD |
| | | ITGLIEGQEYKFRVIAKNKFGCGPPVEIGPILAVDPLGPPTSPERLTYTERTKSTITLDW |
| | | KEPRSDGGSPIQGYIIEKRRHDKPDFERVNKRLCPTTSFLVENLDEHQMYEFRVKAVN |
| | | DVGESEPSLPLNVVIQDDEVPPTIKLRLAVRGDTIKVKAGEPVNIPADVTGLPMPKIE |
| | | WSKNEKVIDKPTDTLNITKEEVSRSEAKTELSIPKAAREDKGTYTITASNRLGSVFRN |
| | | VHVEVYDRPSPPRNLAVTDIKAESCYLTWDAPLDNGGSEITHYIIDKRDASRKKSEW |
| | | EEVTNTAVERRYGIWKLIPNGQYEFRVRAVNKYGISDECKSDKVVIQDPYRLPGPPG |
| | | KPKVLERTKGSMLVSWTPPLDNGGSPITGYWLEKREEGGTYWSRVSRAPITKVGLK |
| | | GVEFNVPRLIEGVKYQFRAMAINAAGIGPPSEPSDPAVAGDPIYPPGPPSCPEVKDKT |
| | | KSSISLAWKPPAKDGGSPIKGYIVEMQEEGTTDWKPVNEPDKLLTACECVVPNLKEL |
| | | RKYRFRVKAVNEAGESEPSDTTGEIPATDIQEVPEVFIDIGAQDCLVCKAGSQVKIPA |
| | | VIKGRPTPKSSWEFDGKAKKAMKDGVHDIPEDAQLETAENSSVIIIPECTRAHSGKYS |
| | | ITAKNKAGQKTANCRVKVMDAPGPPKDLKVSDITRGSCRLSWKMPDDDGGDRIKG |
| | | YVIEKKTIDGKAWTKVNPNCGSTTFVVPDLISEQQYFFRVRAENRFGIGPPAETIQRTT |
| | | ARDPIYPPDPPIKLKIGLITKNTVHLSWKPPKNDGGSPVTHYIVECLAWDPTGKKKEA |
| | | WRQCNRRDVEELEFTVEDLIEGGEYEFRVKAVNEAGVSKPSATVGPVIVKDQTCPPA |
| | | IELKEFMEVEEGTDVNIVAKIKGVPFPTLTWFKAPPKKPDSKEPVVYDTHVNKQVVD |
| | | DTCTLVIPQSRRSDTGLYSITAVNNLGTASKEMRLNVLGRPGPPVGPIKFESISADQM |
| | | TLSWLPPKDDGGSKITNYVIEKREANRKTWVRVSSEPKECMYTIPKLLEGHEYVFRI |
| | | MAQNKYGIGEPLDSEPETARNLFSVPGAPDKPTVSSVTRNSMTVNWEEPEYDGGSPV |
| | | TGYWLEMKDTTSKRWKRVNRDPIKAMTLGVSYKVTGLIEGSDYQFRVYAINAAGV |
| | | GPASLPSDPVTARDPVAPPGPPFPKVTDWTKSSVDLEWSPPLKDGGSKITGYIVEYKE |
| | | EGKEEWEKGKDKEVRGTKLVVTGLKEGAFYKFRVRAVNVAGVGEPGEVTDVIEMK |
| | | DRIVSPDLQLDASVRDRIVVHAGGVIRIIAYVSGKPPPTVTWSMNERALPQEAAIETT |
| | | AISSSMVIKNCQRSHQGVYSLLAKNEGGERKKTIIVDVLDVPGPVGIPFLSDNLTNDS |
| | | CKLTWFSPEDDGGSPITNYVIQKREADRRAWTPVTYTVTRQNATVQGLIQGKSYFFR |
| | | IAAENSIGMGPFVETPNALVIRDPITVPERPEDLEVKEVTKNTVSLTWNPPKYDGGSEI |
| | | INYVLESRLIGTEKFHKVTNDNLLSRKYTVKGLKEGDTYEYRVSAVNIVGQGKPSFC |
| | | TKPITCKDELAPPTLDLDFRDKLTVRVGESFALTGRYSGKPKPKIDWFKDEADVLED |
| | | DRTHIKTTPTTLALEKTKAKRSDSGKYCVVVENSTGSRKGFCQVNVVDRPGPPVGPV |
| | | IFDEVTKEYMVISWKPPLDDGGSEITNYIIEKKELGKDIWMPVTSASAKTTCKVPKLL |
| | | EGKDYIFRIHAENLYGISDPLVSDSMKAKDRFRVPDAPEQPVVTEVTKDSALVTWNK |
| | | PNDGGKPITNYILEKRETMSKRWVRVTKEPIHPYTKYRVPDLLEGCQYEFRVSAENEI |
| | | GIGDPSPPSKPVFARDPIAKPSPPINPEAIDTTCNSVELTWQPPRHDGGSKILGYIVEYQ |
| | | KVGDEEWRRANHTPESCPETKYKVTGLRDGQSYKFRVLAVNEAGESDPAHVPEPVL |
| | | VKDRLEPPELILDANMAREQHIRVGDTLRLSAIIKGVPFPKVTWKKEDREAPTKAQID |
| | | VTPVGSKLEIRNAAHEDGGIYSLTVENPAGTKTVSVKVLVLDKPGPPRDLEVSEIRKD |
| | | SCYLTWKEPLDDGGSVVTNYVVERKDVATAQWSPLSTTSKKKSHMAKHLTEGNQY |
| | | LFRVAAENQYGRGPFVETPKPIKALDPLHPPGPPKDLHHVDVDKTEVSLVWNKPDR |
| | | DGGSPITGYLVEYQEEGDKDWIKFKTVKNLDCVVTGLQQGKTYRFRVKAENIIGLGL |
| | | PDTTIPIECQEKLVPPSVELDVKLIEGLVVKAGTTVRFPAIIRGVPVPTAKWTTDGTEI |
| | | KTDDHYTVETDSFSSVLTIKNCLRKDTGEYQLTVSNAAGTKTVAVHLTVLDVPGPPT |
| | | GPINILDVTPEYMTISWQPPKDDGGSPVINYIVEKQDTRKGTWGVVSAGSSKLKLKV |
| | | PHLQKGCEYVFRVKAENKMGVGPPLDSIPTVAKHKFSPPSPPGKPVVTDITENAATV |
| | | SWTLPKSDGGSPITGYYVERREITGKWVRVNKTPIADLKFRVTGLYEGNTYEFRVFA |
| | | ENLAGLSNPSPSSDPIKACRPIKPPGPPINPKLKDKSKESADLVWTKPLSDGGSPILGY |
| | | VVEYQKPGTAQWDRINKDELIRQCAFRVPGLIEGNEYRFRIRAANIVGEGEPRELAES |
| | | VIAKDILHPPEVELDVTCRDVITVRVGQTIRILARVKGRPEPDITWSKEGKVLVKDKR |
| | | VDLIHDLPRVELQIKEAVRADHGKYIISAKNSSGHAQGSAIVNVLDRPGPCQNLKVSN |
| | | VTKENCTISWENPLDNGGSEITNFIVEYRKPNQKGWSIVASDVTKRLVKANLLANNE |
| | | YYFRVCAENKVGVGPTIETKTPILAINPIDRPGEPENLHIADKGKTFVYLKWRRPDYD |
| | | GGSPNLSYHVERRLKGSADWERVHKGSIKETHYMVDKCVENQIYEFRVQTKNEGGE |
| | | SDWVRTEEVVVKEDLQKPVLDLKLSGVLTVKAGDTIRLEAGVRGKPFPEVAWTKD |
| | | KDATDLTRSPRVKIDTSAESSKFSLTKAKRSDGGKYVITATNPAGSFVAYATVNVLD |
| | | KPGPVRNLKITDVSSDRCTIRWDPPEDDGGCEIQNYILEKCESKRMVWSTYSANVLT |
| | | PSATVTRLIEGNEYIFRVRAENKIGTGPPTESKPVIAKTKYDRPGRPDPPEVTKVSKEE |
| | | MTVVWNAPEYDGGKSITGYYLEKKEKHAVRWVPVNKSAIPERRLKVQNLLPGHEY |
| | | QFRVKAENEVGIGEPSLPSRPVVAKDPIEPPGPPTNFKVVDTTKSSITLAWGKPVYDG |
| | | GAPIIGYVVEMRPKIADASPDEGWKRCNAAAQLIRMEFTVTSLDENQEYEFRVCAQN |
| | | QVGIGRPAELKEAIKPKEILEPPEIDLDASMRKLVVVRAGCPIRLFAIVRGRPAPKVTW |
| | | RKVGIDNVVRKGQVDLVDTMAFLVIPNSTRDDSGKYSLTLVNPAGEKAVFVNVKVL |
| | | DTPGPVADLKVSDVTKTSCHVSWAPPENDGGSQVTHYIVEKREAERKTWSTVTPEV |
| | | KKTSFNVTNLVPGNEYFFRVTAVNEYGPGVPTDIPKPVLASDPLSEPDPPRKLEVTEM |
| | | TKNSATLAWLPPLRDGGAKIDGYIISYREEDQPADRWTEYSVVKDLSLIVTGLKEGK |
| | | KYKFRVAARNAVGVSMPREAEGVYEAKEQLLPPKILMPEQITIKAGKKLRVEAHVY |
| | | GKPNPICKWKKGDDEVVTSSHLAIHKADGSSVLIIKDVTRKDSGYYSLTAENSSGSDT |
| | | QKIKVTVMDAPGPPQPPFDISEIDADACSLSWHIPLEDGGSNITNYIVEKCDVSRGDW |
| | | VTALASVTKTSCRVGKLIPGQEYIFRVRAENRFGISEPLTSPKMLAKFPFDVPSEPKNA |
| | | RVTKVNKDCIFVAWDRPDSDGGSPITGYLIERKERNSLLWVKANDTIVRSTEYPCAG |
| | | LVEGLEYSFRIYALNKAGSSPPSKPTEYVTARMPVDPPGKPEVVDVTKNSASLIWARP |
| | | KHDGGSRIIGYFVEACKLPGDKWVRCNTTPHQIPQEEYTATGLEENAQYQFRAIAKT |
| | | AVNISQPSEPSDPVTILAENVPPRIELSVEMKSLLTVKAGTNVCLDATVFGKPMPTVS |
| | | WKKDTTPIKQAEGIKMAMKRNLCTLELFSVNRKDSGDYTITAENSSGSKSATIKLKV |
| | | LDKPGPPASVKINKMYADRAMLSWEPPLEDGGSEITNYIIDKRETSRPNWAQVSATV |
| | | PITSCTVEKLIEGHEYQFRICAENKYGVGDPILTEPAIAKNPYDPPGRCDPPVISNITKD |
| | | HMTVSWKAPADDGGSPITGYLVEKRETQAVNWTKVNRKPVIERTLKATGLQEGTEY |
| | | EFRVTAINKAGPGKPSDASKAVYAQDPLYPPGPPAFPKVYDTTRSSVSLSWGKPAYD |
| | | GGSPIIGYLVEVKRADSDHWVRCNLPEKLQKTRFEVTGLMENTEYQFRVYAVNKIG |
| | | YSDPSDVPDKHCPKDILIPPEGELDAELRKTLILRAGVTMRLYVPVKGRPPPKITWSK |
| | | PNVNLRERIGLDIKSTDFDTFLRCENVNKYDAGKYILTLENSCGKKEYTIVVKVLDTP |
| | | GPPVNVTVKEVSKDSAYVTWDPPIIDGGSPIINYVVEKRDAERKSWSTVTTECSKTSF |
| | | RVSNLEEGKSYFFRVFAENEYGIGDPGETRDAVKASETPGPVVDLKALAITKSSCTIG |
| | | WKKPRSDGGSRITGYVVDFLTEENKWQRVMKSLSLQYSTKDLKEGKEYTFRVSAEN |
| | | ENGEGTPSEIVVVAKDDVVAPDLDLKDLPDLCYLAKENSNFRLKIPIKGKPAPSVSW |
| | | KKGEDPLATDTRVSVESTAVNTTLVVYDCQKSDAGKYTITLKNVAGTKEGTLSIKV |
| | | VGKPGIPTGPIKFDEVTAEAMTLKWGPPKDDGGSEITNYVLEKRDSVNNKWVTCAS |
| | | AVQKTTFRVTRLHEGIEYTFRVSAENKYGVGEGLKSEPIVAKHPFDVPDAPPPPNIVD |
| | | VRHDSVSLTWTDPKKTGGSPITGYHIEFKERNSLLWKRANKTPIRMKDFKVTGLTEG |
| | | LEYEFRVMAINLAGVGKPSLPSEPVVALDPIDPPGKPEVISVTRNSVTLIWTEPKYDG |
| | | GHKLTGYIVEKRDLPSKSWMKANHVNVPDCAFTVTDLVEGGKYEFRIRAKNTAGAI |
| | | SAPSESTGTIICKDEYEAPTIVLDPTIKDGLTVKAGDSIVLSAISILGKPLPKSSWSRAG |
| | | KDIRPSDIAQITSTPTSSMLTVKYATRKDAGEYTITATNPFGTKEEHVKVSVLDVPGPP |
| | | GPIEISNVSAEKATLTWTPPLEDGGSPIKAYVLEKRETSRLLWTVVSEDIQACRHVVT |
| | | KLIQGNEYLFRVSAVNHYGKGEPVQSEPVKMVDRFGPPGPPGKPEISNVTKNTATVS |
| | | WKRPTDDGGSEITGYHVERREKKGLRWVRATKTPVSDLRCKVTGLQEGNTYEFRVS |
| | | AENRAGIGPPSDASNPVLMKDVAYPPGPPSNAHVTDTTKKSASLAWGKPHYDGGLE |
| | | ITGYVVEHQKVGDDAWIKDTTGTALRITQFVVPDLQTKEKYNFRISAINDAGVGEPA |
| | | VIPNVEIVEKEVAPDFELDAELRRTLVVRAGLSIRIFVPIKGRPAPEVTWTKDNINLKH |
| | | RANIENTESFTLLIIPECNRYDTGKFVMTIENPAGKKSGFVNVRVLDTPGPVLNLRPTD |
| | | ITKDSVTLHWDLPLIDGGSRITNYIVEKREATRKSYSTVTTKCHKCTYKVTGLTEGCE |
| | | YFFRVMAENEYGVGEPTETTEPVRASEAPLPPDSLNIMDITKNTVSLAWPKPRHDGG |
| | | SKITGYVIEAQRKGSDQWTHISTVKGLECVVRNLTEGEEYTFQVMAVNSAGRSAPRE |
| | | SRPVIVKEQTMLPELDLRGIYQKLVIARAGDNIKVEIPVLGRPKPTVTWKKGDQILKQ |
| | | TQRVNVENTATSTILNINECVRSDSGPYPLTAKNTVGEVGDVITIQVHDIPGPPTGPIK |
| | | FDEVSSDFVTFSWEPPENDGGVPISNYVVEMRQTDSTTWVELATTVIRTTYKATRLT |
| | | TGVEYQFRVRAQNRYGVGPGITSASVVANYPFKVPGPPGTPQVTAVTKDSMTISWH |
| | | EPLSDGGSPILGYHIERKERNGILWQTVSKALVPGNIFKSTGLTDGIAYEFRVIAENMA |
| | | GKSKPSKPSEPMFALDPIDPPGKPVPLNITRHTVALKWAKPEYTGGFKITSYVVEKRD |
| | | LPNGRWLKANFSNILENEFTVSGLTEDAAYEFRVIAKNAAGAISPPSEPSDAITCRDDL |
| | | EAPRIMVDVRFKDTITLKAGEAFKLEADVSGRPPPTMEWTKDGKELEGTGKLEIKIA |
| | | DFSTHLINKDSSRTDSGAYILTATNPGGFAKHIFNVKVLDRPGPPEGPLAVSDVTSEK |
| | | CVLSWLPPLDDGGAKIDHYIVQKRETSRLAWTNVATEVQVTKLKVTKLLKGNEYIF |
| | | RVMAVNKYGVGEPLESEPVLAVDPYGPPDPPKNPEVTTITKDSMVVCWGHPDSDGG |
| | | SEIINYIVERRDKAGQRWVKCNKKALTDLRFKVSGLTEGHEYEFRIMAENAAGISAPS |
| | | ATSPFYKACDTVFKPGPPGNPRVLDTSRSSISIAWNKPIYDGGSEITGYMVEIALPEED |
| | | EWQVVTPPAGLKATSYTITSLIENQEYKIRIYAMNSEGLGEPALVPGTPKAEERMLPP |
| | | EIELDADLRKVVTIRACCTLRLFVPIKGRPAPEVKWAREHGESLDKASIESTSSYTLLV |
| | | VGNVNRFDSGKYILTVENSSGSKSAFVNVRVLDTPGPPQNLKIKEVTKTSVTLTWEPP |
| | | LLDGGSKIKNYIVEKRESTRKAYSTVATNCHKTSWKVDQLQEGCSYYFRVLAENEY |
| | | GIGLPAETAESVKASERPLPPGKITLTDVTRNSVSLSWEKPEHDGGSRILGYIVEMQSK |
| | | GSDRWATCATVKVTEATITGLIQGEEYSFRVSAQNEKGISDPRQLSVPVIAKDLVIPP |
| | | AFKLLFNTFTVLAGEDLKIDVPFIGRPPPAVTWHKDDIPLKQTTRVNAESTENNSLLTI |
| | | KEACREDVGHYTVKLTNSAGEATETLNVIVLDKPGPPTGPVKMDEVTADSVTLSWE |
| | | PPKYDGGSSINNYIVEKRDTSTTAWQIVSATVARTTIKACRLKTGCEYQFRIAAENRY |
| | | GKSTYLNSEPVVAQYPFKVPGPPGTPFVTLASKDSMEVQWHEPVSDGGSKVIGYHLE |
| | | RKERNSILWVKLNKTPIPQTKFKTTGLEEGIEYEFRVSAENIVGIGKPSKPSECYAAHD |
| | | PCDPPGRPEAIIVTRNSVTLQWKKPTYDGGSKITGYIVEKKELPDGRWMKASFTNIID |
| | | TQFEVTGLLEDHRYEFRVIARNAAGVFSEPSESTGAITARDEVEPPRISMDPKYRDTV |
| | | VVQAGESFKIDADIYGKPIPTTQWVKGDQELSSTARLEIKSTDFATSLSVKDAVRVDS |
| | | GNYILKAKNVAGEKSVTINVKVLDRPGPPEGPVAISGVTAEKCTLAWKPPLQDGGSD |
| | | ITNYIVERRETSRLVWTLVDANVQTLSCKVLKLLEGNEYIFRIMAVNKYGVGEPLESE |
| | | SLIAKNPFVVPDAPKAPEVTAVTKDSMIVVWERPASDGGSEILGYVLEKRDKEGIRW |
| | | TRCHKRLIGELRLRVTGLLENHNYEFRVSAENAAGLSEPSPPSAYQKACDPIYKPGPP |
| | | NNPKVMDVTRSSVFLSWTKPIYDGGCEIQGYIVEKCDVSVGEWTMCTPPTGINKTNL |
| | | EVEKLLEKHEYNFRICAINKAGVGEHADVPGPVMVEEKLEAPDIDLDLELRKVINIRA |
| | | GGSLRLFVPIKGRPTPEVKWGKVDGDIRDAAIIDVTSSFTSLVLDNVNRYDSGKYTLT |
| | | LENSSGTKSAFVTVRVLDTPSPPVNLKVTEITKDSVSITWEPPLLDGGSKIKNYIVEKR |
| | | EATRKSYAAVVTNCHKNSWKIDQLQEGCSYYFRVTAENEYGIGLPARTADPIKVAE |
| | | VPQPPGKITVDDVTRNSVSLSWTKPEHDGGSKIIQYIVEMQAKNTDKWSECARVKSL |
| | | DAVITNLTQGEEYLFRVIAVNEKGRSDPRSLAVPIIAKDLVIEPDVRPAFSSYSVQVGQ |
| | | DLKIEVPISGRPKPSISWTKDGMPLKQTTRINVTDSLDLTTLSIKETHKDDGGQYGITV |
| | | SNVVGQKTAAIEIITLDKPDPPKGPVKFDEISAESITLSWNPPLYTGGCQITNYIVQKR |
| | | DTTTTVWDVVSATVARTTLKVTKLKTGTEYQFRIFAENRYGQSFALESEPVVAQYPY |
| | | KEPGPPGTPFVTAISKESMVVQWHEPINNGGSPVIGYHLERKERNSILWTKVNKTIIH |
| | | DTQFKALNLEEGIEYEFRVYAENIVGVGKASKNSECYVARDPCDPPGTPEAIIVKRNE |
| | | ITLQWTKPVYDGGSMITGYIVEKRDLPEGRWMKASFTNVIETQFTVSGLTEDQRYEF |
| | | RVIAKNAAGAISKPSDSTGPITAKDEVELPRISMDPKFRDTIVVNAGETFRLEADVHG |
| | | KPLPTIEWLRGDKEIEESARCEIKNTDFKALLIVKDAIRIDGGQYILRASNVAGSKSFP |
| | | VNVKVLDRPGPPEGPVQVTGVTAEKCTLAWSPPLQDGGSDISHYVVEKRETSRLAW |
| | | TVVASEVVTNSLKVTKLLEGNKYIFRIMAVNKYGVGEPLESAPVLMKNPFVLPGPPK |
| | | SLEVTNIAKDSMTVCWNRPDSDGGSEIIGYIVEKRDRSGIRWIKCNKRRITDLRLRVT |
| | | GLTEDHEYEFRVSAENAAGVGEPSPATVYYKACDPVFKPGPPTNAHVVDTTKNSITL |
| | | AWSKPIYDGGSEILGYVVEICKADEEEWQIVTPQTGLRVTRFEIAKLIEHQEYKIRVC |
| | | ALNKVGLGEAASVPGTVKPEDKLEAPELDLDSELRKGIVVRAGGSARIHIPFKGRPTP |
| | | EITWSKEEGEFTDKVQIEKGINFTQLSIDNCDRNDAGKYILKLENSSGSKSAFVTVKV |
| | | LDTPGPPQNLAVKEVRKDSVLLVWEPPIIDGGAKVKNYVIDKRESTRKAYANVSSKC |
| | | NKTSFRVENLTEGAIYYFRVMAENEFGVGVPTETSDAVKASEPPSPPGKVTLTDVSQ |
| | | TSASLMWEKPEHDGGSRILGYVVEMQPKGTEKWSVVAESKVCNAVVSGLSSGQEY |
| | | QFRVKAYNEKGKSDPRVLGIPVIAKDLTIQPSFKLPFNTYSVQAGEDLKIEIPVIGRPRP |
| | | KISWVKDGEPLKQTTRVNVEETATSTILHIKESSKDDFGKYSVTATNSAGTATENLSV |
| | | IVLEKPGPPVGPVKFDEVSADFVVISWEPPAYTGGCQISNYIVEKRDTTTTNWQMVS |
| | | ATVARTTIKISKLKTGTEYQFRIFAENRYGKSTPLDSKPVVVQYPFKEPGPPGTPFVTS |
| | | ISKDQMLVQWHEPVNDGGSKVTGYHLEQKEKNSILWVKLNKIPIQDTKFKTTGLDE |
| | | GLEYEFRVSAENIVGIGKPSKVSECYVARDPCDPPGRPEAIVITRNSVTLKWKKPVYD |
| | | GGSKITGYIVEKKDLPDGRWMKASFTNVVETEFTVTGLVEDQRYEFRVIARNAADN |
| | | FSEPSESSGAITARDEIDAPNASLDPKYRDVIIVHAGETFVLEADIRGKPIPDIIWSKDG |
| | | NELEETAARMEIKSTLQKTTLIVKDCIRTDGGQYTLKLSNVGGTKTIPITVKVLDRPG |
| | | PPEGPLKVTGVTAEKCYLAWNPPLQDGGASISHYIIEKRETSRLSWTQVSNEVQALN |
| | | YKVTKLLPGNEYIFRVMAVNKYGIGEALESEPVIACNPYKRPGPPSTPEASAITKDSM |
| | | VLTWTRPVDDGGAEIEGYILEKRDKEGIRWTKCNKKTLTDLRFRVTGLTEGHSYEFR |
| | | VAAENAAGVGEPSEPSVFYRACDALYPPGPPSNPKVTDTSRSSVSLAWNKPIYDGGA |
| | | PVRGYVIELKKAAADEWTTCTPPSGLQGKQFTVTKLKENTEYNFRICAFNTEGVGEP |
| | | ATIPGSVVAQERMEAPEIELDADLRKVVTLRASATLRLFVTIKGRPEPEVKWEKAEGI |
| | | LTERAQIEVTSSYTMLVIDNVTRFDSGRYNLTLENNSGSKTAFVNVRVLDSPSAPVNL |
| | | TIREVKKDSVTLSWEPPLIDGGAKITNYIVEKRETTRKAYATITNNCTKNTFKIENLQE |
| | | GCSYYFRVLASNEYGIGLPAETAEPVKVSEPPLPPGRVTLVDVTRNTATIKWEKPESD |
| | | GGSKITGYVVEMQTKGSEKWSACTQVKTLETTISGLTAGEEYVFRVAAVNEKGRSD |
| | | PRQLGVPVIAKDIEIKPSVELPFNTFNVKANDQLKIDIPFKGRPQATVAWKKDGQVLR |
| | | ETTRVNVASSKTVTTLSIKEASREDVGTYELCVSNTAGSITVPITVIVLDRPGPPGPIRI |
| | | DEVSCDNVSISWNPPEYDGGCQISNYIVEKRETTSTTWQVVSQAVARTSIKIVRLTTG |
| | | SEYQFRVCAENRYGKSSYSESSAVVAEYPFSPPGPPGTPKVVHATKSTMVVSWQVPV |
| | | NDGGSQVIGYHLEYKERSSILWSKANKVLIADTQMKVSGLDEGLMYEYRVYAENIA |
| | | GIGKCSKACEPVPARDPCDPPGQPEVTNITRKSVSLKWSKPRYDGGAKITGYIVERRE |
| | | LPDGRWLKCNFTNVQETYFEVTELTEDQRYEFRVFARNAADSVSEPSESTGPITVKD |
| | | DVEAPRIMMDVKFRDVIIVKAGEVLKINADIAGRPLPVISWAKDGVEIEERAKTEIVS |
| | | TDYTTTLTVKDCVRRDTGQYVLTLKNVAGTRTMAVNCKVLDKPGPPAGPLEINGLT |
| | | AEKCSLSWGRPQEDGGADIDYYIVEKRETSRLAWTICEAELRTTSCKVTKLLKGNEY |
| | | IFRVTGVNKYGVGEPLESMAVKALDPFTTPSPPTSLEITSVTKDSMTLCWSRPETDGG |
| | | SDISGYIIERREKNSLRWMRVNKKPVYDLRVKSTGLREGCEYEYRVFAENAAGLSLP |
| | | SETSPLVRAEDPVFLPSPPSKPKIVDSGKTTITIGWVKPLFDGGAPITGYTVEYKKSEET |
| | | DWKVAIQSFRGTEYTMSGLTTGDEYVFRVRSLNKMGASDPSDSSDPQVAKEREEEP |
| | | VFDVDSEMRKTLIVKAGSSFTMTVPFRGRPIPNVSWSKPDTDLRTRAYIDSTDSRTLL |
| | | TIENANRNDSGKYTLTIQNVLSAASMTFVVKVLDSPGPPANITVREVTKETAMLSWD |
| | | VPENDGGAPVKNYHIEKREASKKAWVSVTNNCNRLSYKVTNLQEGAIYYFRVSGEN |
| | | EFGVGVPAETKEGVKITEKPSPPEKLGVTSVSKDSVSLSWLKPEHDGGSRIIHYVVEA |
| | | LEKGQKTWVKCAVVKTTHHVVSGLRESHEYFFRVFAENQAGLSDPRELLLPVLIKD |
| | | QLEPPEIDMKNFPSHTVYVRAGSNLKVDIPISGKPLPKVTLSRDGVPLKATMRENTEIT |
| | | AENLTINLKESVTTDAGRYEITAANSSGTTKTFINIIVLDRPGPPTGPVAISDITEESVTL |
| | | KWEPPKYDGGSHVTNYIVLKRETSTAVWSEVSATVARTMIKVMKLTTGEEYQFRIK |
| | | AENREGISDHIDSVCVVVKLPYTTPGPPSTPWVSNVTRESITVGWHEPVSNGGSAVTG |
| | | YHLEMKDRNSILWQKANKMIIRTTHFKVTTISAGLIYEFRVYAENAAGIGKPSHPSEP |
| | | VLAIDACEPPRNVRITDISKNSVNLSWQQPAFDGGSKITGYIVERRDLPDGRWTKASF |
| | | TNVIETQFTVSGLTQNSQYEFRVFARNAVGSVSNPSEVVGPITCIDSYGGPVIDLPLEY |
| | | TEVVKYRAGTSVKLRAGISGKPEPTIEWYKDDKELQTNALVCVENSTDLASILIKDA |
| | | NRLNSGSYELKLRNAMGSASATIRVQILDKPGPPGGPIEFKTVTAEKITLLWRPPADD |
| | | GGAKITHYIVEKRETSRVVWSMVAENLEECIVTTTKIIKGNEYVFRVRAVNKYGIGEP |
| | | LESEPVVAKNAFVTPGPPSIPEVTKITKNSMTVVWDRPTVDGGSEINGYFLERRDKKS |
| | | LAWLKVLKETIRDTRQKVTGLTENSDYQYRVCAVNAAGVGPFSEPSDFYKAADPID |
| | | PPGPPAKIRIADSTKSSITLGWSKPVYDGGSDVTGYVVEMKQGDEEEWTIVSTRGEV |
| | | RTTEYVVSNLKPGVNYYFQVSAVNCAGQGEPITMTEPAQAKDVLEEPEIDLDVALRT |
| | | SVIAKAGEDVQLLIPFKGRPPPTVTWRKDEKNLGSDTRYSIQNTDSSSLLVIPQVTRN |
| | | DTGKYILTIENGVGQPKSSTVSVKVLDTPAACQKLQVKHVSLGTVTLLWDPPLIDGG |
| | | SPIINYVIEKRDATKRTWSVVSHKCSGTSFKVTDLSEKTPFFFRVLAENEIGIGEPCETT |
| | | EPVKAAEVPAPIRDLSMKDSTKTSVVLSWTKPDFDGGSIITDYLVERKGKGEQAWSH |
| | | AGISKTCEIEIGQLKEQSVLEFRVSARNEKGQSDPVTIGPLTVKELVITPEVDLSEIPGA |
| | | QISVRIGHNVHLELPYKGKPKPSISWLKDGLPLKESEYVRFSKTENKITLSIKNSKKEH |
| | | GGKYTVILDNAVCRNSFPITIITLGPPSKPKGPIRFDEIKADSAIMSWDIPEDDGGGEIT |
| | | CYSIEKREASQTNWKMVCSSVARTTFKVSNLVKDSEYQFRVRAENRYGVSEPLASNI |
| | | IVAKHQFRIPGPPGKPVIYNVTSDGMSLTWDAPVYDGGSEVTGFHVEKKERNSILWQ |
| | | RVNTSPISGREYRATGLIEGLDYQFRVYAENSAGLSSPSDPSKFTLAVSPVDPPGTPDY |
| | | IDVTRETITLKWNPPLRDGGSKIVAYSIEKRQGSDRWVRCNFTDVSECQYTVTGLSPG |
| | | DRYEFRIIARNAVGTISPPSQSSGLIMTRDENVPPTVEFGPEYFDGLVIKSGDSLRIKAL |
| | | VQGRPVPRVTWFKDGVEIERRMNMEITDVLGSTSLFVRDATRDHRGVYTVEAKNVS |
| | | GSTKAEVTVKVQDTPGKVVGPIRFTNITGEKMTLWWEAPLNDGCAPVTHYIIEKRET |
| | | SRLAWALIEDNCEALSYTAIKLITGNEYQFRISAVNKFGVGRPLESDPVVAQIQYTIPD |
| | | APGVPEPSNVTGNSITLTWTRPESDGGSEIQHYILERREKKSTRWVKVISKRPISETRF |
| | | KVTGLVEGNEYEFHVMAENAAGIGPASGISRLIKCREPVNPPSAPSVVKVTDTSKTTV |
| | | SLEWARPVFDGGMEIIGYIIEMCKADLGDWHKVNTEPCVKTRYTVTDLQAGEEYKF |
| | | RVSAVNGAGKGDSCEVTGTIKAVDRLSAPELDIDANFKQTHIVRAGVSIRLFIAYQGR |
| | | PTPTAVWSKPDSNLSIRADIHTTDSFSTLTVENCNRNDAGKYTLTVENNSGKKSITFT |
| | | VKVLDSPGPPGPITFKDVTRGSATLMWDAPLLDGGARIHHYVIEKREASRRSWQVVS |
| | | EKCTRQILKVSELTEGVPYYFRVSAENEYGVGEPYEMPEPIVATEQPAPPRRLDVVDT |
| | | SKSSAVLAWLKPDHDGGSRITSYLLEMRQKGSDFWVEAGHTKQLTFTVERLVENTE |
| | | YEFRVKAKNDAGYSEPREAFSSVIIKEPQIEPTADLTGITNQLITCKAGSTFTIDVPISG |
| | | RPAPKVTWKLEEMRLKETDRMSIATTKDRTTLTVKDSMRGDSGRYFLTLENTAGVK |
| | | TFTITVVVIGRPGPVTGPIEVSSVSAESCVLSWTEPKDDGGTEITNYIVEKRESGTTAW |
| | | QLINSSVKRTQIKVTHLTKYKEYCFRVSSENRFGVSKPLESVPIVAEHPFVPPSAPTRP |
| | | EVYYVSANAMSIRWEEPYHDGGSKIVGYWVEKKERNTILWVKENKVPCLECNYKV |
| | | TGLVEGLEYQFRTYALNAAGVSKASEASRPIMAQNPVDPPGRPEVTDVTRSTVSLIW |
| | | SAPVYDGGSKVVGYIIERKPVSEVGDGRWLKCNYTIVSDNFFTVTALSEGDTYEFRV |
| | | LAKNAAGVISKGSESTGPVTCRDEYAPPKAELDARLQGDLVTIRAGSDLVLDAAVG |
| | | GKPEPKIIWTKGDKELDLCEKISLQYTGKRATAVIKYCDRSDSGKYTLTVKNASGTK |
| | | SVSVMVKVLDSPGPCGKLTVSRVTEEKCTLAWSLPQEDGGAEITHYIVERRETSRLN |
| | | WVIVEGECLTASYVVTRLIKNNEYTFRVRAVNKYGLGVPVESEPIVARNSFTIPSQPGI |
| | | PEEVGAGKEHIIIQWTKPESDGGNEISNYLVDKREKKSLRWTRVNKDYVVYDTRLK |
| | | VTSLMEGCDYQFRVTAVNAAGNSEPSEASNFISCREPSYTPGPPSAPRVVDTTKRSISL |
| | | AWTKPMYDGGTDIIGYVLEMQEKDTDQWCRVHTNATIRNNEFTVPDLKMGQKYSF |
| | | RVAAVNAKGMSDYSETTAEIEPVERLEIPDLELADDLKKTVIVRAGASLRLMVSVSG |
| | | RPSPVITWSKKGIDLANRAIIDNTESYSLLIVDKVNRYDAGKYTIEAENQSGKKSATV |
| | | LVKVYDTPGPCPSVSVKEVSRDSVTITWEIPTIDGGAPVNNYIIEKREAAMRAFKTVT |
| | | TKCSKTLYRISGLVEGTMYYFRVLPENIYGIGEPCETSDAVLVSEVPLVPTKLEVVDV |
| | | TKSTVTLAWEKPLYDGGSRLTGYVLEACKAGTERWMKVVTLKPTVLEHTVISLNEG |
| | | EQYLFRVRAQNEKGVSEPREIVTPVTVQDLRVLPTIDLSTMPQKTIHVPAGRPIELVIPI |
| | | TGRPPPTASWFFAGSKLRESERVTVETHTKVTKLTIRETTIRDTGEYTLELKNVTGTTS |
| | | ETIKVIILDKPGPPTGPIKIDEIDATSVTISWEPPELDGGAPLSGYVVEQRDAHRPGWLP |
| | | VSESVTRPTFKFTRLTEGNEYVFRVAATNRFGIGSYLQSEVIECRSSISIPGPPETLQIFD |
| | | VSRDGMTLTWYPPEDDGGSQVTGYIVERKEVRADRWVRVNKVPVTMTRYRSTGLI |
| | | EGLEYEHRVTAINARGTGKPSRPSKPTVAMDPIAPPGKPQNPRVTDTTRTSVSLAWS |
| | | VPEDEGGSKVTGYLIEMQKVDQREWTKCNTTPTKIREYTLTHLPQGAEYRFRVLAC |
| | | NAGGPGEPAEVPGTVKVTEMLEYPDYELDERYQEGVFVRQGGVIRLTIPIKGKPFPV |
| | | CKWTKEGQDISKRAMIATSETHTELVIKEADRNDSGTYDLVLENKCGKKTVYIKVK |
| | | VIGSPNTPEGPLEYDDIQARSVRVSWRPPADDGGADILGYILERREVPKAAWYTIDSR |
| | | VRGTSLVVKGLKENVEYHFRVSAENQFGISKPLKSEEPVIPKTPLNPPEPPSNPPEVLD |
| | | VTKSSVSLSWSRPKDDGGSRVTGYYIERKETSTDKWVRHNKTQITTTMYTVTGLVP |
| | | DAEYQFRIIAQNDVGLSETSPASEPVVCKDPFDKPSQPGELEILSISKDSVTLQWEKPE |
| | | CDGGKEILGYWVEYRQSGDSAWKKSNKERIKDRQFTIGGLLEATEYEFRVFAENETG |
| | | LSRPRRTAMSVKTKLTSGEAPGVRKEMADVTTKLGEAAQLSCQIVGRPLPDIKWYRF |
| | | GKELIQSRKYKMSSDGRTHTLTVMTDEQEDEGVYTCVATNEVGEVESSSKLLLQAA |
| | | PQFHPGYPLKEKYYGAVGSTLRLHVMYIGRPVPAMTWFHGQKLLQNSEKITIENTEH |
| | | YTHLVMKNVQRKTHAGKYKVQLSNAFGTVDATLDVEIQDKPDKPTGPIVIEALLKN |
| | | SVVISWKPPADDGGSWITNYVVEKCEAKEGAEWQLVSSAISVTTCRIVNLTENAGYY |
| | | FRVSAQNTFGISEPLEVASIVIIKSPFEKPGVPGKPTITAVTKDSCVVAWKPPASDGGA |
| | | KIRNYYLERREKKQNKWIAVTTEEIRETVFSVQNLIEGLEYEFRVKCENLGGESEWSE |
| | | ISEPVTPKSDVPIQAPHFKEELRNLNVRYQSNATLVCKVTGHPKPIVKWYRQGKEIIA |
| | | DGLKYRIQEFKGGYHQLIIASVTDDDATVYQVRATNQGGSVSGTASLEVEVPAKIHL |
| | | PKTLEGMGAVHALRGEVVSIKIPFSGKPDPVITWQKGQDLIDNNGHYQVIVTRSFTSL |
| | | VFSNGVERKDAGFYVVCAKNRFGIDQKTVELDVADVPDPPRGVKVSDVSRDSVNLT |
| | | WTEPASDGGSKVTNYIVEKCATTAERWLRVGQARETRYTVINLFGKTSYQFRVIAEN |
| | | KFGLSKPSEPSEPTVTKEDKTRAMNYDDEVDETREVTTTKASHSKTKELYEKYMIAE |
| | | DLGRGEFGIVHRCVETSSKRTFMAKFVKVKGTDQVLVKKEISILNIARHRNILYLHES |
| | | FESMEELVMIFEFISGLDIFERINTSAFELNEREIVSYVRQVCEALEFLHSQNIGHFDIRP |
| | | ENIIYQTRKNSTIKIIEFGQARQLKPGDNFRLLFTAPEYYAPEVHQHDVVSTATDMWS |
| | | LGTLVYVLLSGINPFLAETNQQMIENIMNAEYTFDEEAFKEISLEAMDFVDRLLVKER |
| | | KSRMTASEALQHPWLKQRIDRVSTKVIRTLKHRRYYHTLIKKDLNMVVSAARISCGG |
| | | AIRSQRGVSVAKVKVASIEIGPVSGQIMHAIGEEGGYVKYVCKIENYDQSTQVTWYF |
| | | GVRQLENSEKYEITYEDGVATMYVKDITKFDDGTYRCKVVNDYGEDSSYAELFVKG |
| | | VREVYDYYCRRTKKVKRRTDAMRLLERPPEFTLPLYNKTAYVGENVRFGVTITVHP |
| | | EPRVTWYKSGQKIKPGDDEKKYTFESDKGLYQLTINSVTTDDDAEYTVVARNKHGE |
| | | DSCKAKLTVTLHPPPTETTLRPMFKRLLANAECHEGQSVCFEIRVSGIPAPTLKWEKD |
| | | GQPLSLGPHIEIVHEGLDYYALHIRDTLPEDTGYYRVTATNTAGSTSCQAHLQVERLR |
| | | YVKQEFQSKEERERHVQKQIDKTLRMAEILSGTETVPLTPVAQEALREAAILYKPAVS |
| | | TKTVKGEYRLQTEEKKEERKLRMPYEVPEPRRFKQATVEEDQRIKQFVPMSDMKWY |
| | | KKIRDQYEMPGKLDRVVQKRPKRIRLSRWEQFYVMPLPRITDQYRPKWRIPKLTQD |
| | | DLEMVRPARRRTPSPDYDLYYYRRRRRSLGDMSDEELLLPIDDYLAMKRTEEERLRL |
| | | EEELELGFSASPPSRSPPRFELSSLRYSSPPAHVKVEDRRRDFRYSTYHVPTKEETSTS |
| | | YAELRERHAQASYRQPKLRQRIMAEKEEEELLRPVTTTQRLSEYKSELDYMSKEEKS |
| | | KKKSKRQRQVTEITEIEEEYEISRRAQRESSSSVSRLLRRRRSLSPTYIELMRPVSELIRS |
| | | HPRPAEEYEDDAERRSPTPERTRPRSPSPVSSERSLSRFERSARFDIFSRYESMKAALK |
| | | TQKTSERKYEVLSQQPFTLDHAPRITLRMRSHRVPCGQNTRFILNVQSKPTAEVKWY |
| | | HNGVELQESSKIHYTNTSGVLTLEILDCQTEDGGTYRAVCTNYKGEASDYATLDVTG |
| | | GAYTTYASQRRDEEVPKSVFPELTKTEAYAVSSFKRTSELEAASSVREVKSQMTETR |
| | | ESLSTYEHYASAEMKSATSEEKSLEEKATVRKIKTTLAARILTKPRSITVHEGESARFS |
| | | CDTDGEPVPTVTWLREGQVVSTSARHQVTTTKYKSTFEISSVQASDEGNYSVVVENS |
| | | DGKQEAQFTLTVQKARVIEKAVTSPPRVKSPEPRVKSPETVKSPKRVKSPEPVTSHPK |
| | | AVSPTETKPTEKGQHLPVSAPPKITQSLKAEASKDIAKLTCAVESSALCAKEVAWYK |
| | | DGKKLKENGHFQFHYSADGTYELKIHNLSESDCGEYVCEVSGEGGTSKTSFQFTGQS |
| | | FKSIHEQVSSISETTKSVQKTAESPEAKKQEPIAPESISSKPVIVTGLRDTTVSSDSVAKF |
| | | TIKVTGEPQPTITWTKDGKAIAQGSKYKLSSNKEEFILEILKTETSDGGLYACTVTNSA |
| | | GSVSSSCKLTIKAVKDTEAQKVSTQKTSEVTSQKKASAQEEISQKALTSEEIKMSEVK |
| | | SHETLAIKEEASKVLIAEEVKRSAAASLEKSIVHEEVTKTSQASEEVKTHAEIKTLSTQ |
| | | MNITKGQRATLKANIAGATDVKWVLNGTELPNSEEYRYGVSGSDQTLTIKQASHRE |
| | | EGILSCIGKTSQGVVKCQFDLTLSEELSDAPSFITQPRSQNINEGQNVLFSCEVSGEPSP |
| | | EIEWFKNNLPISISSNISVSRSRNVYTLEIRNAAVSDSGKYTIKAKNFHGQCSATASLT |
| | | VLPLVEEPPREVVLKTSSDVSLHGSVSSQSVQMSASKQEASFSSFSSSSASSMTEMKF |
| | | ASMSAQSMSSMQESFVEMSSSSFMGKSSMTQLESSTSRMLKAGGRGIPPKIEALPSDI |
| | | SIDEGKVLTVACAFTGEPTPEITWSCGGRKIQNQEQQGRFHIENTDDLTTLIIMDVQK |
| | | QDGGLYTLSLGNEFGSDSATVNINIRSM |
| 2 | Mouse MYH4 | MSSDAEMAVFGEAAPYLRKSEKERIEAQNKPFDAKSSVFVVDAKESYVKATVQSRE |
| | (Myosin-4) | GGKVTAKTEGGATVTVKDDQVFSMNPPKYDKIEDMAMMTHLHEPAVLYNLKERY |
| | | AAWMIYTYSGLFCVTVNPYKWLPVYNPEVVAAYRGKKRQEAPPHIFSISDNAYQFM |
| | | LTDRENQSILITGESGAGKTVNTKRVIQYFATIAVTGDKKKEEATSGKMQGTLEDQII |
| | | SANPLLEAFGNAKTVRNDNSSRFGKFIRIHFGATGKLASADIETYLLEKSRVTFQLKA |
| | | ERSYHIFYQIMSNKKPELIEMLLITTNPYDFAYVSQGEITVPSIDDQEELMATDTAVDI |
| | | LGFSADEKVAIYKLTGAVMHYGNMKFKQKQREEQAEPDGTEVADKAAYLTSLNSA |
| | | DLLKALCYPRVKVGNEYVTKGQTVQQVYNSVGALAKSMYEKMFLWMVTRINQQL |
| | | DTKQPRQYFIGVLDIAGFEIFDFNTLEQLCINFTNEKLQQFFNHHMFVLEQEEYKKEGI |
| | | DWEFIDFGMDLAACIELIEKPMGIFSILEEECMFPKATDTSFKNKLYEQHLGKSNNFQ |
| | | KPKPAKGKAEAHFSLVHYAGTVDYNIIGWLDKNKDPLNETVVGLYQKSGLKTLAFL |
| | | FSGGQAAEAEGGGGKKGGKKKGSSFQTVSALFRENLNKLMTNLKSTHPHFVRCLIP |
| | | NETKTPGAMEHELVLHQLRCNGVLEGIRICRKGFPSRILYADFKQRYKVLNASAIPEG |
| | | QFIDSKKASEKLLGSIDIDHTQYKFGHTKVFFKAGLLGTLEEMRDEKLAQLITRTQAV |
| | | CRGYLMRVEFKKMMERRESIFCIQYNVRAFMNVKHWPWMKLYFKIKPLLKSAETE |
| | | KEMANMKEDFEKAKEDLAKSEAKRKELEEKMVALMQEKNDLQLQVQAEADGLAD |
| | | AEERCDQLIKTKIQLEAKIKELTERAEDEEEINAELTAKKRKLEDECSELKKDIDDLEL |
| | | TLAKVEKEKHATENKVKNLTEEMAGLDENIAKLTKEKKALQEAHQQTLDDLQAEE |
| | | DKVNTLTKAKTKLEQQVDDLEGSLEQEKKLRMDLERAKRKLEGDLKLAQESTMDIE |
| | | NDKQQLDEKLKKKEFEMSNLQSKIEDEQALGMQLQKKIKELQARIEELEEEIEAERA |
| | | SRAKAEKQRSDLSRELEEISERLEEAGGATSAQIEMNKKREAEFQKMRRDLEEATLQ |
| | | HEATAAALRKKHADSVAELGEQIDNLQRVKQKLEKEKSELKMEIDDLASNMETVSK |
| | | AKGNLEKMCRTLEDQLSEVKTKEEEQQRLINELSTQKARLHTESGEFSRQLDEKDA |
| | | MVSQLSRGKQAFTQQIEELKRQLEEESKAKNALAHALQSARHDCDLLREQYEEEQE |
| | | AKAELQRAMSKANSEVAQWRTKYETDAIQRTEELEEAKKKLAQRLQDAEEHVEAV |
| | | NSKCASLEKTKQRLQNEVEDLMIDVERSNAACAALDKKQRNFDKVLAEWKQKYEE |
| | | TQAELEASQKESRSLSTELFKVKNAYEESLDQLETLKRENKNLQQEISDLTEQIAEGG |
| | | KHIHELEKIKKQIDQEKSELQASLEEAEASLEHEEGKILRIQLELNQVKSEIDRKIAEKD |
| | | EEIDQLKRNHLRVVESMQSTLDAEIRSRNDALRIKKKMEGDLNEMEIQLNHANRQA |
| | | AEAIRNLRNTQGMLKDTQLHLDDALRGQDDLKEQLAMVERRANLMQAEIEELRAS |
| | | LEQTERSRRVAEQELLDASERVQLLHTQNTSLINTKKKLETDISQIQGEMEDIVQEAR |
| | | NAEEKAKKAITDAAMMAEELKKEQDTSAHLERMKKNMEQTVKDLQHRLDEAEQL |
| | | ALKGGKKQIQKLEARVRELENEVENEQKRNIEAVKGLRKHERRVKELTYQTEEDRK |
| | | NVLRLQDLVDKLQTKVKAYKRQAEEAEEQSNVNLAKFRKIQHELEEAEERADIAES |
| | | QVNKLRVKSREVHTKVISEE |
| 3 | Mouse | MADEEEYEEVIEYYTEETVYEEVPGETITEVYETTTTRTSDYGQSETATPALAQPQPA |
| | E9Q1W3 | KPVERKKVIRKKVDSSKFMTPYIQHSQKMQDLFSSNKYKENYEKAKGQPYAITSDTP |
| | (Nebulin) | ELRRIKKVQDQLSEVKYRMDGDVAKTICHVDEKAKDIEHAKKVSQQVSKVLYKQN |
| | | WEDTKDKYLLPPDAPELVQAVKNTAMFSKKLYTEDWEADKALFYPYNDSPELRRV |
| | | AQAQKALSDIAYKKGLTEQQTQFTSLPDPPEVEFAKKVTNQVSKQKYKEDYEKKVK |
| | | GKWSETPCFEIATARMNADNLSSRKYQEDFENMKDQIYFMQTETPEYKMNKQAGV |
| | | AASKVKYKQDYEKNKGKADYNVLPASENPLLRQLKTAGNVLSDKLYKENYEKTKA |
| | | RSINYCETPKFQLDTVLQNFSSDTKYKDSYLKNILGHYVGSFEDPYHTHCMKVTAQN |
| | | SDKNYKAEYEEDRGKGFFPQTITQEYEAIKKLDQCKDHAYKVHPDKTKFTQVTDSP |
| | | VLVQAQVNSKQLSDLNYKAKHESEKFKCHIPPDAPAFIQHRVNAYNLSDNVYKQDW |
| | | EKSKAKKFDIKVDAIPLLAAKANSKNASDVMYKKDYEKSKGKMIGALSINDDPKML |
| | | HSLKTAKNQSDRLYRENYEKTKAKSMNYCETPKYQLDTLLKNFSEAKYKDSYVKN |
| | | MLGHYVGSFEDPYHTHCMKVSAQNSDKSYKAEYEEDKGKCYFPQTITQEYESIKKL |
| | | DQCKDHTYKVHPDKTRFTAVTDTPVLLQAQLNTKQLSDLNYKAKHEGEKFKCSVP |
| | | ADAPEFIQHRVNAYNLSDNVYKHDWEKSKAKKFDIKVDAIPLLAAKANSKNASDV |
| | | MYKKDYEKSKGKMIGALSINDDPKMLHSLKTAKNQSDREYRKDYEKSKTIYTAPLD |
| | | MIQVTQAKKSQAIASDVDYKHVLHNYSYPPDSINVDLAKKAYALQSDVEYKADYNS |
| | | WMKGCGWVPFGSLEMEKAKRASEILNEKKYRQHPDTLKFTSIEDAPITVQSKINQAQ |
| | | RSDIAYKAKGEEILHKYNLPADLPQFIQAKVNAYNISENMYKADLKDLSKKGYDLRI |
| | | DAIPIKAAKAARQAASDIHYKKDYEKAKGKMVGFQSLQDDPKLVHYMNVAKIQSD |
| | | REYKKDYEKTKTKYNTPHDMFDVVAAKKAQDVISNTNYKHSLHHYTYLPDAMDLE |
| | | LSKNMMHIQSDNAYKEDYNTWMKGIGWIPIGSLDQEKVKKAGDALSEKKYRQHPD |
| | | TLKFTSIVDSPVMVQAKQNTQQASDILYKAKGEDVKHKYTLNPDLPQFLQAKCNAY |
| | | NISDVCYKRDWHDLIAKGNNVLADAIPITAAKSSRNIASDYKYKEAYEKAKGKHVG |
| | | FRSLQDDPKLVHYMNVAKLQSDREYKKNYENTKTSYHTPGDMVSITAARMAQDVA |
| | | TNVNYKQPLHHYTYLPDALSLEHTRNANQIQSDNVYKDEYNSFFKGIGWIPIGSLEV |
| | | EKAKKAGEALNERKYRQHPDTIKFTSVPDSMGMVLAQQNTKQLSDLNYKVEGEKM |
| | | KHKYTMDPELPQFIQAKVNAINMSDAHYKADWKKTLAKGYDLRPDAIPIVAAKSSR |
| | | NIASDCKYKEAYEKARGKQIGFLSLQDDPKLVHYMNVAKIQSDREYKKGYEASKTR |
| | | YHTPLDMFSVTAAKKSQEVATNSNYRQPYHNYTLLPDALNVEHSRNAMQIQSDNLY |
| | | KSDFTHWMKGIGWVPIDSLEVEKAKKAGEILSERKYRQHPEKLKFTYSMDTMEQAL |
| | | NKSNKLTMDKRLYTEKWNKDKTSIHVMPDTPDILLSRVNQITMSDKLYKAGWEEEK |
| | | TKGYDLKPDAIAIKAAKASRDIASDYKYKQAYEQSKGKHIGFRSLEDDPKLVHFMQ |
| | | VAKIQSDREYKKGYEKSKTSFHTPVDMFSVVAAKKSQEVATNANYRNIIHTYNMLP |
| | | DAMSFELAKNMMQIQSDNQYKADYADFMKGIGWLPLGSLEAEKNKKAMEIISEKK |
| | | YRQHPDTLKYSTLMDSMNMVLAQNNAKIMNKNLYKQAWEADKTKIHIMPDIPEIVL |
| | | AKANAINMSDKLYKLSLEESRKKGYDLRTDAIPIKAAKASRDIASDYKYKHSYEKEK |
| | | GKMVGFRSLEDDPKLVHSMQVAKMQSDREYKKNYEKTKTSYHTPADMLSVTAAK |
| | | DAQANITNTNYKRLIHKYILLPDAMNIELTRNMNHIQSDNEYKQDYREWYRGLGWS |
| | | PAGSLEVEKAKKATEYASDQKYRQHPSTFQFKKLTDSMDMVLAKQNAHTMNKHLY |
| | | TIDWNKDKTKIHVMPDTPDILQAKQNQTLYSQKSYRLGWEEALKKGYDLPVDAISV |
| | | QLAKTSRDIASDFKYKQGYRKQLGHHIGFLSVQDDPKLVLSMNVAKMQSDREYKK |
| | | DFEKWKTKYTSPVDMLGVVLAKKCQALVSDADYRNYLHQWTCLPDQNDVIQAKK |
| | | VYELQSENMYKSDLEWLRGIGWSPLGSLEAEKNKRASEIISEKKYRQPPDRNKFTSIP |
| | | DAMDIVLAKTNAKNRSDRLYREAWDKDKTQIHIMPDTPDIILAKANLINTSDKLYKM |
| | | GYEELKKKGYDLPVDAIPIKAAKASREIASEYKYKEGFRKQLGHHIGARNIKDDPKM |
| | | MWSMHVAKIQSDREYKKDFEKWKTKYSSPVDMLGVVLAKKCQTLVSDADYRNYL |
| | | HQWTCLPDQNDVIHARQAYDLQSDNVYKADLQWLKGIGWIPSGSLEDEKNKRATQI |
| | | LSDHVYRQHPNKFKFSSLMDSMPMVLAKNNAITMNHHLYTEAWNKDKTTVHIMPD |
| | | TPEVILAKQNKVNYSEKLYKLGLEEAKRKGYDMRLDAIPIKTAKASRDIASDFKYKE |
| | | GYRKQLGHHIGARGIHDDPKMMWSMHVAKIQSDREYKKDFEKWKTKYSSPVDML |
| | | GVVLAKKCQTLVSDADYRNYLHQWTCLPDQNDVIHARQAYDLQSDNMYKSDLQW |
| | | MRGIGWVPIGSLDVEKCKRATEILSDKIYRQPPDKFKFTSVTDSLEQVLAKNNAITMN |
| | | KRLYTEAWDKDKTQIHIMPDTPEIMLARMNKVNYSESLYKLANEEAKKKGYDLRSD |
| | | AIPIVAAKASRDIASDYKYKDGYRKQLGHHIGARNIKDDPKMMWSMHVAKIQSDRE |
| | | YKKDFEKWKTKYSSPVDMLGVVLAKKCQILVSDIDYKHPLHEWTCLPDQNDVIHAR |
| | | QAYDLQSDNVYKSDLQWMRGIGWVPIGSLDVVKCKRAAEILSDNIYRQPPNKFKFT |
| | | SVTDSLEQVLAKSNALNMNKRLYTEAWDKDKIQIHVMPDTPEIMLARQNRINYSESL |
| | | YKLANEEAKKKGYDLRVDAIPIVAAKASRDIASDYKYKVGYRKQLGHHIGARNIED |
| | | DPKMMWSMHVAKIQSDREYKKDFEKWKTKYSSPVDMLGVVLAKKCQTLVSDVDY |
| | | RNYLHQWTCLPDQNDVIHARQAYDLQSDNVYKSDLQWMRGIGWVPIGSLDVVKCK |
| | | RAAEILSDNIYRQPPNKFKFTSVTDSLEQVLAKSNALNMNKRLYTEAWDKDKTQIHI |
| | | MPDTPEIMLARQNKINYSESLYRQAMEEAKKEGYDLRSDAIPIVAAKASRDIASDYK |
| | | YKEAYRKQLGHHIGARAIHDDPKMMWSLHIAKVQSDREYKKEFEKYKTRYSSPVD |
| | | MLGIVLAKKCQTLVSDVDYKHPLHEWTCLPDQNDVIQARKAYDLQSDNLYKADLE |
| | | WMKGIGWVPIGSLEVVKAKRATEILSDNIYRQRPDTLKFTSITDSLEQVLAKNNAITM |
| | | NKRLYTEAWDNDKKTIHVMPDTPEIMLAKLNRINYSDKLYKVSLEEARRQGYDLRV |
| | | DAIPIRSAKASREIASDYKYKEGYRKQLGHHIGARNIKDDPKMMWSMHVAKIQSDR |
| | | EYKKDFEKWKTKYSSPVDMLGVVLAKKCQILVSDIDYKHPLHEWTCLPDQNDVIQA |
| | | RKAYDLQSDAVYKADLEWLRGIGWVPIGSVEVEKVKRAGEILSDRKYRQPADQLKF |
| | | TCITDTPEIVLAKSNAITMSKHLYTEAWDADKASIHVMPDTPEILLAKSNSANISHKL |
| | | YTKGWDESKMKDYDLREDAISIKSAKASRDIASDYKYKEAYEKQKGHHIGAQSVED |
| | | DPRILCAMHAGKIYSDREYKKEFQKWKTKFSSPVDMLGVVLAKKCQTLVSDVDYR |
| | | NYLHNWTCLPDQNDIIQAKKAYELQSDSVYKADLEWLRGIGWMPEGSIEMTRVKG |
| | | AQDLVNERLYRTRPEALKFTSIVDTPEVVLAKANSLQMSERLYQEAWNKDKTNISLP |
| | | LDTPAMLQAQINALQISNKLYQKDWDEAKQKGYDLRADAIEIKHAKASREIASEYK |
| | | YKEGYRKQLGHHVGFRSLQDDPKLVWSIHAAKIQSDREYKKAYEKSKGIYNTPLDM |
| | | MSIVQAKKCQVLVSDVDYRNYLHQWTCLPDQNDVIQAKKAYELQSDAIYKADLEW |
| | | LRGIGWMPEGSPEVLRVKNAQEILCDSVYRTPVVKLKYTSIVDTPEVVLAKSNAENIS |
| | | IPKYREVWDKDKTSIHIMPDTPEINLARANALNVSNKLYREGWDEVKMSCDVRLDAI |
| | | PIQAAKASREIASDYKYKLDHEKQKGHYVGTLTARDDNKIRWALIAGKIQNEREYRL |
| | | QWAKWKTKFQSPVDMLSILHSKKCQTLVSDIDYRNYLHEWTCMPDQNDVIQAKKA |
| | | YELQSDAVYKADLEWLRGIGWMPNDSVSVNHAKHAADIFSEKKYRTKIETLNFTPV |
| | | DDRVDYVTAKQSSEIRDDIKYRKDWNAIKSKYTLTETPLLHTAQEAARILDQYLYKE |
| | | GWEKQKATGYILPPDAVPFVHAHHSSDVQSELKYKAEHVKQRGHYVGVPTMRDDP |
| | | KLVWFEHAGQIQNDRLYKESYHKSKAKINIPADMVSVVAAKEGQNLVSDIDYRQYL |
| | | HQWTCHPDQNDVIQAKIAYDLQSDNVYKADLEWLRGIGWIPLDSVDHVRVTKNQE |
| | | MVNQIKYKKDALANYPNFTSVEDPPEIVLAKINSVNQSDVKYKETFNKAKGKYIFSP |
| | | DTPYISHSKDMGKLYSTILYKGAWEGTKAYGYTLDERYIPIVGAKHADLVNSELKYK |
| | | ETYEKQKGHYLAGKEISEFPGVVHCLDFQQMRSALNYRRDYEDTKANVHIPNDMM |
| | | NHVLAKRCQYILSDLEYRHYFHQWTSLPEEPNVIRARNAQEILSDNVYKDDLNWLK |
| | | GIGCYVWDTPQILHAKKSYDLQSQLQYTAASKENLQNYNLVTDTPVYVTALQSGIN |
| | | ASEVKYKENYHQIKDKYTTVLETADYDRTKHLKSLYSSNIYKEAWDKVKATSYNLP |
| | | TSTLSLTHAKNQKHLASHIKYREEYEKFKALYTLPKSVDDDPNTARCLRVGKLNIDR |
| | | LYRSVYEKNKMKINIVPDMVEMVTAKDSQKKVSEIDYRLHLHEWICHPDLQVNSHV |
| | | RKVTDQISDIVYKDDLTWLKGIGCYVWDTPEILHAKHAYDLRNDIKYKAHVQKTRN |
| | | DYKLVTDTPVYVQAVKSGKQLSDAVYHYDYVHSVRGKVAPTTKTVDLDRALHAY |
| | | KLQSENLYKKGLRFLPTGYRLPVDTPHFNHTKDTRYMSSYFKYKEAYEHIKANGYT |
| | | LGPKDVPFVHVRRVNNVTSERLYRELYHKLKDKIHTTPDTPEIRQVKKTQEAVSELIY |
| | | KSDFFKMQGHMISLPYTPQVLHCRYVGDITSDIKYKEDLQILKGLGCFLYDTPDMVR |
| | | SRHLRKLWSNYLYTDNARKMRDKYKVVLDTPEYRKVQELKTHLSELVYRASGKKQ |
| | | KSIFTSVPDTPDLLRAKRGQKLQSQYLYVELATKERPHHHAGNQTTALQHAKDVKD |
| | | MVSEKKYKIQYEKMKDKYTPVADTPILIRAKRAYWNASDLRYKETFQKTKGRYHT |
| | | VKDALDIVYHRKVTDDISKVKYKENYMSQLGIWSSIPDRPEHFHHRAVTDAVSDVK |
| | | YKEDLTWLKGIGCYAYDTPDLTLAEQNKTLYSKYKYKEVFERTKSNFKYVADCPIN |
| | | RHFKYATQLMNEKKYRADYEQRKDKYHLVVDEPRHLLAKTAGDQISQIKYRKKYE |
| | | KSKDKFTSVVDTPEHLRTTKVNKQISDILYKLEYNKARPRGYTTIHDTPMLLHVRKV |
| | | KDEVSDLKYKEVYQRNKSNCTIKPDAVHIKAAKDAYKVNTNLDYKKQYEANKAY |
| | | WKWTPDRPDFIQAAKSSLQQSDFEYKLDREFLKGCKLSVTDDKDMVLALRNSLIESD |
| | | LKYKEKHVKERGSCHAVPDTPQILLAKTVSNLVSENKYKDHVKKHLAQGSYTTLPE |
| | | TRDTVHVKAVTKHVSDTNYKKKFVKEKGKSNYSIMQEPPEVKHAMEVAKKQSDVA |
| | | YKKDAKENLHYTTVADRPDIKKATQAAKQASEVEYRAKHRKEGSHGLSMLGRPDIE |
| | | MAKKAAKLSSQVKYRENFDKEKGKTPKYNPKDSQLYKTMKDANNLASEVKYKAD |
| | | LKKLHKPVTDMKESLIMHHVLNTSQLASSYQYKKNYEKSKGHYHTIPDNLEQLHHR |
| | | EATELQSIVKYKEKYEKERGKPMLDFETPTYITAKESQQMQSGKEYRKDYEESIKGR |
| | | NLTGLEVTPALLHVRHATKIASEKEYRKDLEESIRGRGLTEMEDTPDMLRAKNATQI |
| | | LNEKEYKRDLELEVKGRGLNAMANETPDFMRARNATDIASQIKYKQSAEMEKANFT |
| | | SVVDTPEIIHAQQVKNLSSQKKYKEDAEKSMSYYETVLDTPEMQRVRENQKNFSLL |
| | | QYQCDLKNSKGKITVVQDTPEILRVKENQKNFSSVLYKEDVSPGTAIGKTPEMLRVK |
| | | QTQDHISSVKYKEAIGQGTPIPDLPEVKRVKETQKHISSVMYKENPGTGIPTTVTPEIE |
| | | RVKRNQENFSSVLYKESLGKGTPTAITPEMERVKRNQENFSSVLYKENMGKGTPLPV |
| | | TPEMERVKHNQENISSVLYKENVGKATPTPVTPEMQRVKRNQENISSVLYKENMGK |
| | | ATPTPFTPEMERVKRNQENFSSVLYKENMRKATPTPVTPDMERAKRNQENISSVLYS |
| | | DSFRKQIQGKAAYVLDTPEMRRVRETQRHISTVKYHEDFEKHKGCFTPVVTDPITER |
| | | VKKNTQDFSDINYRGIQRKVVEMEQKRNDQDQDTITGLRVWRTNPGSVFDYDPAED |
| | | NIQSRSLHMINVQAQRRSRDQSRSASALSISGGEEKSEHSEAADQRLSTYSDGGAFFS |
| | | ATSTAYKHVKTTQLPQQRSSSVATQQTTLSSIPSHPSTAGKIFRAIYDYIAADADEVSF |
| | | KDGDAIVNAQAIDEGWMYGTVQRTGRTGMLPANYVEAI |
| 4 | Mouse ACTS | MCDEDETTALVCDNGSGLVKAGFAGDDAPRAVFPSIVGRPRHQGVMVGMGQKDSY |
| | (Actin) | VGDEAQSKRGILTLKYPIEHGIITNWDDMEKIWHHTFYNELRVAPEEHPTLLTEAPLN |
| | | PKANREKMTQIMFETFNVPAMYVAIQAVLSLYASGRTTGIVLDSGDGVTHNVPIYEG |
| | | YALPHAIMRLDLAGRDLTDYLMKILTERGYSFVTTAEREIVRDIKEKLCYVALDFEN |
| | | EMATAASSSSLEKSYELPDGQVITIGNERFRCPETLFQPSFIGMESAGIHETTYNSIMK |
| | | CDIDIRKDLYANNVMSGGTTMYPGIADRMQKEITALAPSTMKIKIIAPPERKYSVWIG |
| | | GSILASLSTFQQMWITKQEYDEAGPSIVHRKCF |
| 5 | Mouse KCRM | MPFGNTHNKFKLNYKPQEEYPDLSKHNNHMAKVLTPDLYNKLRDKETPSGFTLDDV |
| | (Creatine | IQTGVDNPGHPFIMTVGCVAGDEESYTVFKDLFDPIIQDRHGGYKPTDKHKTDLNHE |
| | Kinase) | NLKGGDDLDPNYVLSSRVRTGRSIKGYTLPPHCSRGERRAVEKLSVEALNSLTGEFK |
| | | GKYYPLKSMTEQEQQQLIDDHFLFDKPVSPLLLASGMARDWPDARGIWHNDNKSFL |
| | | VWVNEEDHLRVISMEKGGNMKEVFRRFCVGLQKIEEIFKKAGHPFMWNEHLGYVL |
| | | TCPSNLGTGLRGGVHVKLANLSKHPKFEEILTRLRLQKRGTGGVDTAAVGAVFDISN |
| | | ADRLGSSEVEQVQLVVDGVKLMVEMEKKLEKGQSIDDMIPAQK |
| 6 | Mouse ACTN3 | MMMVMQPEGLGAGEGPFSGGGGGEYMEQEEDWDRDLLLDPAWEKQQRKTFTAW |
| | (Alpha-Actinin- | CNSHLRKAGTQIENIEEDFRNGLKLMLLLEVISGERLPRPDKGKMRFHKIANVNKAL |
| | 3) | DFIASKGVKLVSIGAEEIVDGNLKMTLGMIWTIILRFAIQDISVEETSAKEGLLLWCQR |
| | | KTAPYRNVNVQNFHTSWKDGLALCALIHRHRPDLIDYAKLRKDDPIGNLNTAFEVA |
| | | EKYLDIPKMLDAEDIVNTPKPDEKAIMTYVSCFYHAFAGAEQAETAANRICKVLAVN |
| | | QENEKLMEEYEKLASELLEWIRRTVPWLENRVGEPSMSAMQRKLEDFRDYRRLHKP |
| | | PRVQEKCQLEINFNTLQTKLRLSHRPAFMPSEGKLVSDIANAWRGLEQVEKGYEDW |
| | | LLSEIRRLQRLQHLAEKFQQKASLHEAWTRGKEEMLNQHDYESASLQEVRALLRRH |
| | | EAFESDLAAHQDRVEHIAALAQELNELDYHEAASVNSRCQAICDQWDNLGTLTQKR |
| | | RDALERMEKLLETIDQLQLEFARRAAPFNNWLDGAIEDLQDVWLVHSVEETQSLLT |
| | | AHEQFKATLPEADRERGAILGIQGEIQKICQTYGLRPKSGNPYITLSSQDINNKWDTV |
| | | RKLVPSRDQTLQEELARQQVNERLRRQFAAQANAIGPWIQGKVEEVGRLAAGLAGS |
| | | LEEQMAGLRQQEQNIINYKSNIDRLEGDHQLLQESLVFDNKHTVYSMEHIRVGWEQ |
| | | LLTSIARTINEVENQVLTRDAKGLSQEQLNEFRASFNHFDRKRNGMMEPDDFRACLIS |
| | | MGYDLGEVEFARIMTMVDPNAAGVVTFQAFIDFMTRETAETDTAEQVVASFKILAG |
| | | DKNYITPEELRRELPAEQAEYCIRRMAPYKGSGAPSGALDYVAFSSALYGESDL |
| 7 | Mouse MYH1 | MSSDAEMAVFGEAAPYLRKSEKERIEAQNKPFDAKSSVFVVDAKESFVKATVQSRE |
| | (Myosin-1) | GGKVTAKTEGGTTVTVKDDQVYPMNPPKYDKIEDMAMMTHLHEPAVLYNLKERY |
| | | AAWMIYTYSGLFCVTVNPYKWLPVYNAEVVAAYRGKKRQEAPPHIFSISDNAYQFM |
| | | LTDRENQSILITGESGAGKTVNTKRVIQYFATIAVTGEKKKEEATSGKMQGTLEDQIIS |
| | | ANPLLEAFGNAKTVRNDNSSRFGKFIRIHFGTTGKLASADIETYLLEKSRVTFQLKAE |
| | | RSYHIFYQIMSNKKPDLIEMLLITTNPYDYAFVSQGEITVPSIDDQEELMATDSAIDILG |
| | | FTSDERVSIYKLTGAVMHYGNMKFKQKQREEQAEPDGTEVADKAAYLQNLNSADL |
| | | LKALCYPRVKVGNEYVTKGQTVQQVYNSVGALAKAVYEKMFLWMVTRINQQLDT |
| | | KQPRQYFIGVLDIAGFEIFDFNSLEQLCINFTNEKLQQFFNHHMFVLEQEEYKKEGIE |
| | | WEFIDFGMDLAACIELIEKPMGIFSILEEECMFPKATDTSFKNKLYEQHLGKSNNFQK |
| | | PKPAKGKVEAHFSLVHYAGTVDYNIAGWLDKNKDPLNETVVGLYQKSSMKTLAYL |
| | | FSGAAAAAEAESGGGGGKKGAKKKGSSFQTVSALFRENLNKLMTNLRSTHPHFVRC |
| | | IIPNETKTPGAMEHELVLHQLRCNGVLEGIRICRKGFPSRILYADFKQRYKVLNASAIP |
| | | EGQFIDSKKASEKLLGSIDIDHTQYKFGHTKVFFKAGLLGLLEEMRDDKLAQLITRTQ |
| | | AMCRGYLARVEYQKMVERRESIFCIQYNVRAFMNVKHWPWMKLYFKIKPLLKSAE |
| | | TEKEMANMKEEFEKAKENLAKAEAKRKELEEKMVALMQEKNDLQLQVQSEADSL |
| | | ADAEERCDQLIKTKIQLEAKIKEVTERAEDEEEINAELTAKKRKLEDECSELKKDIDD |
| | | LELTLAKVEKEKHATENKVKNLTEEMAGLDETIAKLTKEKKALQEAHQQTLDDLQA |
| | | EEDKVNTLTKAKIKLEQQVDDLEGSLEQEKKIRMDLERAKRKLEGDLKLAQESTMD |
| | | VENDKQQLDEKLKKKEFEMSNLQSKIEDEQALGMQLQKKIKELQARIEELEEEIEAE |
| | | RASRAKAEKQRSDLSRELEEISERLEEAGGATSAQIEMNKKREAEFQKMRRDLEEAT |
| | | LQHEATAATLRKKHADSVAELGEQIDNLQRVKQKLEKEKSEMKMEIDDLASNMEVI |
| | | SKSKGNLEKMCRTLEDQVSELKTKEEEQQRLINELTAQRGRLQTESGEYSRQLDEKD |
| | | SLVSQLSRGKQAFTQQIEELKRQLEEEIKAKSALAHALQSSRHDCDLLREQYEEEQEA |
| | | KAELQRAMSKANSEVAQWRTKYETDAIQRTEELEEAKKKLAQRLQDAEEHVEAVN |
| | | AKCASLEKTKQRLQNEVEDLMIDVERTNAACAALDKKQRNFDKILAEWKQKYEET |
| | | HAELEASQKESRSLSTELFKIKNAYEESLDHLETLKRENKNLQQEISDLTEQIAEGGK |
| | | RIHELEKIKKQIEQEKSELQAALEEAEASLEHEEGKILRIQLELNQVKSEIDRKIAEKDE |
| | | EIDQLKRNHIRVVESMQSTLDAEIRSRNDAIRLKKKMEGDLNEMEIQLNHSNRMAAE |
| | | ALRNYRNTQGILKDTQLHLDDALRGQEDLKEQLAMVERRANLLQAEIEELRATLEQ |
| | | TERSRKIAEQELLDASERVQLLHTQNTSLINTKKKLETDISQIQGEMEDIVQEARNAEE |
| | | KAKKAITDAAMMAEELKKEQDTSAHLERMKKNLEQTVKDLQHRLDEAEQLALKG |
| | | GKKQIQKLEARVRELEGEVENEQKRNVEAIKGLRKHERRVKELTYQTEEDRKNVLR |
| | | LQDLVDKLQSKVKAYKRQAEEAEEQSNVNLAKFRKIQHELEEAEERADIAESQVNK |
| | | LRVKSREVHTKIISEE |
| 8 | Mouse PYGM | MSRPLSDQDKRKQISVRGLAGVENVSELKKNFNRHLHFTLVKDRNVATPRDYYFAL |
| | (glycogen | AHTVRDHLVGRWIRTQQHYYEKDPKRIYYLSLEFYMGRTLQNTMVNLALENACDE |
| | Phosphorylase) | ATYQLGLDMEELEEIEEDAGLGNGGLGRLAACFLDSMATLGLAAYGYGIRYEFGIFN |
| | | QKICGGWQMEEADDWLRYGNPWEKARPEFTLPVHFYGRVEHTSQGAKWVDTQVV |
| | | LAMPYDTPVPGYRNNVVNTMRLWSAKAPNDFNLKDFNVGGYIQAVLDRNLAENIS |
| | | RVLYPNDNFFEGKELRLKQEYFVVAATLQDIIRRFKSSKFGSRDPVRTNFDAFPDKVA |
| | | IQLNDTHPSLAIPELMRILVDLERLDWDKAWDVTVKTCAYTNHTVLPEALERWPVH |
| | | LMETLLPRHLQIIYEINQRFLNRVAAAFPGDVDRLRRMSLVEEGAVKRINMAHLCIA |
| | | GSHAVNGVARIHSEILKKTIFKDFYELEPHKFQNKTNGITPRRWLVLCNPGLAEVIAE |
| | | RIGEDYISDLDQLRKLLSYVDDEAFIRDVAKVKQENKLKFSAYLEREYKVHINPNSLF |
| | | DVQVKRIHEYKRQLLNCLHIITLYNRIKREPNRFMVPRTIMIGGKAAPGYHMAKMIIK |
| | | LITAIGDVVNHDPAVGDRLRVIFLENYRVSLAEKVIPAADLSEQISTAGTEASGTGNM |
| | | KFMLNGALTIGTMDGANVEMAEEAGEENFFIFGMRVEDVERLDQRGYNAQEYYDRI |
| | | PELRQIIEQLSSGFFSPKQPDLFKDIVNMLMHHDRFKVFADYEEYIKCQDKVSELYKN |
| | | PREWTRMVIRNIATSGKFSSDRTIAQYAREIWGVEPSRQRLPAPDEKI |
| 9 | Mouse ALDOA | MPHPYPALTPEQKKELSDIAHRIVAPGKGILAADESTGSIAKRLQSIGTENTEENRRFY |
| | (fructose- | RQLLLTADDRVNPCIGGVILFHETLYQKADDGRPFPQVIKSKGGVVGIKVDKGVVPL |
| | bisphosphate | AGTNGETTTQGLDGLSERCAQYKKDGADFAKWRCVLKIGEHTPSALAIMENANVLA |
| | aldolase A) | RYASICQQNGIVPIVEPEILPDGDHDLKRCQYVTEKVLAAVYKALSDHHVYLEGTLL |
| | | KPNMVTPGHACTQKFSNEEIAMATVTALRRTVPPAVTGVTFLSGGQSEEEASINLNAI |
| | | NKCPLLKPWALTFSYGRALQASALKAWGGKKENLKAAQEEYIKRALANSLACQGK |
| | | YTPSGQSGAAASESLFISNHAY |
| 10 | Mouse MYL 1 | MAPKKDVKKPAAAPAPAPAPAPAPAKPKEEKIDLSAIKIEFSKEQQEDFKEAFLLFDR |
| | (myosin light | TGECKITLSQVGDVLRALGTNPTNAEVKKVLGNPSNEEMNAKKIEFEQFLPMMQAIS |
| | chain) | NNKDQGGYEDFVEGLRVFDKEGNGTVMGAELRHVLATLGEKMKEEEVEALLAGQE |
| | | DSNGCINYEAFVKHIMSV |
| 11 | Mouse G3P | MVKVGVNGFGRIGRLVTRAAICSGKVEIVAINDPFIDLNYMVYMFQYDSTHGKFNG |
| | (glyceraldehyde- | TVKAENGKLVINGKPITIFQERDPTNIKWGEAGAEYVVESTGVFTTMEKAGAHLKGG |
| | 3-phosphate | AKRVIISAPSADAPMFVMGVNHEKYDNSLKIVSNASCTTNCLAPLAKVIHDNFGIVE |
| | dehydrogenase) | GLMTTVHAITATQKTVDGPSGKLWRDGRGAAQNIIPASTGAAKAVGKVIPELNGKL |
| | | TGMAFRVPTPNVSVVDLTCRLEKPAKYDDIKKVVKQASEGPLKGILGYTEDQVVSC |
| | | DFNSNSHSSTFDAGAGIALNDNFVKLISWYDNEYGYSNRVVDLMAYMASKE |
| 12 | Mouse TPM1 | MDAIKKKMQMLKLDKENALDRAEQAEADKKAAEDRSKQLEDELVSLQKKLKGTE |
| | (tropomyosin | DELDKYSEALKDAQEKLELAEKKATDAEADVASLNRRIQLVEEELDRAQERLATAL |
| | alpha-1) | QKLEEAEKAADESERGMKVIESRAQKDEEKMEIQEIQLKEAKHIAEDADRKYEEVAR |
| | | KLVIIESDLERAEERAELSEGKCAELEEELKTVTNNLKSLEAQAEKYSQKEDKYEEEI |
| | | KVLSDKLKEAETRAEFAERSVTKLEKSIDDLEDELYAQKLKYKAISEELDHALNDMT |
| | | SI |
| 13 | Mouse MYPC2 | MPEAKPAAKKASKGKDAPKEAPAKQTPEEPPKEAPPEDQSPTAEEPTGIFLKKPDSVS |
| | (myosin-binding | VETGKDAVILAKVNGKELPGKPTIKWFKGKWQELGSKSGARFIFKESHDSTSNVYTV |
| | protein C) | ELHIGKVVLGDRGDYRLEIKAKDVCDSCSFNVDVEAPRQDSSGQSLESFKRSGDGKS |
| | | EDAGELDFSGLLKKREVVEEEKKKKKDDDDLGIPPEIWELLKGAKKSEYEKIAFQYG |
| | | ITDLRGMLKRLKKAKVEVKKSAAFTKKLDPAYQVDRGNKIKLVVEISDPDLPLKWF |
| | | KNGQEIKPSSKYVFENVGKKRILTINKCTLADDAAYEVAVQDEKCFTELFVKEPPVLI |
| | | VTPLEDQQVFVGDRVEMSVEVSEEGAQVMWMKDGVEMTREDSYKARYRFKKDGK |
| | | RHILIYSDVAQEDGGRYQVITNGGQCEAELIVEEKQLEVLQDIADLTVKAAEQAVFK |
| | | CEVSDEKVTGKWYKNGVEVRPSKRITISHVGRFHKLVIDDVRPEDEGDYTFVPDGYA |
| | | LSLSAKLNFLEIKVEYVPKQEPPKIHLDCSGKTSDNSIVVVAGNKLRLDVAITGEPPPT |
| | | ATWLRGDEVFTATEGRTHIEQRPDCSSFVIESAERSDEGRYTIKVTNPVGEDVASIFLR |
| | | VVDVPDPPEAVRVTSVGEDWAILVWEPPKYDGGQPVTGYLMERKKKGSQRWMKIN |
| | | FEVFTDTTYESTKMIEGVLYEMRVFAVNAIGVSQPSMNTKPFMPIAPTSAPQHLTVED |
| | | VTDTTTTLKWRPPDRIGAGGIDGYLVEYCLEGSEEWVPANKEPVERCGFTVKDLPTG |
| | | ARILFRVVGVNIAGRSEPATLLQPVTIREIVEQPKIRLPRHLRQTYIRKVGEALNLVIPF |
| | | QGKPRPQVVWTKGGAPLDTSRVNVRTSDFDTVFFVRQAARSDSGEYELSVQIENMK |
| | | DTATIRIRVVEKAGPAENVMVKEVWGTNALVEWQPPKDDGNSEITGYFVQKADKK |
| | | TMEWFNVYEHNRHTSCTVSDLIVGNEYYFRIFSENICGLSDSPGVSKNTARILKTGITL |
| | | KPLEYKEHDFRTAPKFLTPLMDRVVVAGYTAALNCAVRGHPKPKVVWMKNKMEIH |
| | | EDPKFLITNYQGILTLNIRRPSPFDAGTYSCRAFNELGEALAECKLDVRVPQ |
| 14 | Mouse MYPC3 | MPEPGKKPVSAFNKKPRSAEVTAGSAAVFEAETERSGVMVRWQRDGSDITANDKY |
| | (myosin-binding | GLAAEGKRHTLTVRDASPDDQGSYAVIAGSSKVKFDLKVTEPAPPEKAESEVAPGAP |
| | protein C) | EEVPAPATELEESVSSPEGSVSVTQDGSAAEHQGAPDDPIGLFLMRPQDGEVTVGGSI |
| | | VFSARVAGASLLKPPVVKWFKGKWVDLSSKVGQHLQLHDSYDRASKVYLFELHITD |
| | | AQTTSAGGYRCEVSTKDKFDSCNFNLTVHEAIGSGDLDLRSAFRRTSLAGAGRRTSD |
| | | SHEDAGTPDFSSLLKKRDSFRRDSKLEAPAEEDVWEILRQAPPSEYERIAFQHGVEAC |
| | | HRPLKRLKGMKQDEKKSTAFQKKLEPAYQVNKGHKIRLTVELADPDAEVKWLKNG |
| | | QEIQMSGSKYIFESVGAKRTLTISQCSLADDAAYQCVVGGEKCSTELFVKEPPVLITR |
| | | SLEDQLVMVGQRVEFECEVSEEGAQVKWLKDGVELTREETFKYRFKKDGRKHHLII |
| | | NEATLEDAGHYAVRTSGGQSLAELIVQEKKLEVYQSIADLAVGAKDQAVFKCEVSD |
| | | ENVRGVWLKNGKELVPDNRIKVSHIGRVHKLTIDDVTPADEADYSFVPEGFACNLSA |
| | | KLHFMEVKIDFVPRQEPPKIHLDCPGSTPDTIVVVTGNKLRLDVPISGDPAPTVVWQK |
| | | TVTQGKKASAGPHPDAPEDAGADEEWVFDKKLLCETEGRVRVETTKDRSVFTVEGA |
| | | EKEDEGVYTVTVKNPVGEDQVNLTVKVIDVPDAPAAPKISNVGEDSCTVQWEPPAY |
| | | DGGQPVLGYILERKKKKSYRWMRLNFDLLRELSHEARRMIEGVAYEMRVYAVNAV |
| | | GMSRPSPASQPFMPIGPPGEPTHLAVEDVSDTTVSLKWRPPERVGAGGLDGYSVEYC |
| | | QEGCSEWTPALQGLTERRSMLVKDLPTGARLLFRVRAHNVAGPGGPIVTKEPVTVQ |
| | | EILQRPRLQLPRHLRQTIQKKVGEPVNLLIPFQGKPRPQVTWTKEGQPLAGEEVSIRNS |
| | | PTDTILFIRAARRTHSGTYQVTVRIENMEDKATLILQIVDKPSPPQDIRIVETWGFNVA |
| | | LEWKPPQDDGNTEIWGYTVQKADKKTMEWFTVLEHYRRTHCVVSELIIGNGYYFRV |
| | | FSHNMVGSSDKAAATKEPVFIPRPGITYEPPKYKALDFSEAPSFTQPLANRSIIAGYNA |
| | | ILCCAVRGSPKPKISWFKNGLDLGEDARFRMFCKQGVLTLEIRKPCPYDGGVYVCRA |
| | | TNLQGEAQCECRLEVRVPQ |
| 15 | Mouse Q14BI5 | MSLVAVPFYQKRHKHFDQSYRNIQTRYLLDQYALKKQATTQSSSQRSLTERSSSKRA |
| | (myomesin 2) | SSQSSAGAMTCRLCAKRVSASEEEEVENENRYRSQAASYGEAKRQRFLSELAQLEEN |
| | | VHLARSQARDKLDKYFMEQTVEDNLAWERHSFEDRMSRAPEILVRLRSHTIWERMS |
| | | VRLCFTVQGFPTPVVQWYKDGSLICQAGEPGKYRIESRYGVHTLEINRANFEDTATY |
| | | SAVATNSHGQVSTNAAVVVRRYRGEEEPFHSVGLPIGLPLSSVIPYTHFDVQFLEKFG |
| | | VTFRREGETVTLKCTLLVTPDLKRVQPRAEWYRDDVLLKESKWTKMFFGEGQASLS |
| | | FSHLNKDDEGLYTLRIVSRGGVSDHSAFMFVRDADPLVTGAPGAPMDLQCHDANRD |
| | | YVIVTWKPPNTTTESPVIGYFIDKCEVGTNNWVQCNDAPVKICKYPVTGLFEGRSYV |
| | | FRVRAVNNAGISRPSRISDAVAALDPVDLRRLQAIHLEGEKEIVIYQDDLEGDVQIPGP |
| | | PTNVQASEVSRNYVVLSWDPPSPRGKDPLMYFIEKSAVGSGSWQRVNAQTAVRSPR |
| | | YAVFDLAEGKSYVFRVLSANKHGLSDPSEITPPIQAQDMIVVPSAPGRVLASRNTKTS |
| | | VVVQWDRPKHEEDLLGYYVDCCVAGTNMWEPCNHKPIGYNRFVVHGLTTGEQYIF |
| | | RVKAVNAVGTSENSQESEVIKVQAALTVPSHPYGITLLNCDGHSMTLGWKVPKFSG |
| | | GSAIIGYYLDKREVHHKNWHEVNSSPVKERILTVEGLTEGSLYEFKIAATNLAGIGQP |
| | | SDPSEHFKCEAWTDPEPGPAYDLTFCEVRDTSLVILWKAPVYSGSSPVSGYFVDFKEE |
| | | DSGEWKTTSEAATPNRYLKVCDLQQGKTYVFRIRAVNASGPGKPSDTSEPVLVEARP |
| | | GTKEISAGVDEEGNIYLGFDCQEMTDASQFTWCKAYEEIADEERFEVHTEGDHSKLY |
| | | FKNPDKEDLGTYSVSVSDTDGVSSSFVLDEEELERLMALSNEIKNPTIPLKSELAYEIF |
| | | DKGQVRFWLQAEHLSPDASFRFIINDREVSDSDTHRIKCDRSTGMIEMVMDRFTIENE |
| | | GTYTVQIQDGKAKNQSSLVLIGDAFKAVLEEAEFQRKEFLRKQGPHFAEYLHWDVT |
| | | EECEVRLVCKVANTKRETVFKWLKDDVLYETETPPDLEKGVCELLIPKLSKKDHGE |
| | | YKATLKDDRGQDVSVLEVGGKVYEDMILAMSRVCGASASPLKVLCTPEGIRLQCFM |
| | | KYFTEEMKVSWYHKEAKISSSEHMRIGGSEEMAWLQICEPTEKDKGKYTFEIFDGKD |
| | | SHQRSLDLSGQAFDEAYAEFQQLKAAAFAEKNRGKVIGGLPDVVTIMEGKTLNLTC |
| | | TVFGNPDPEVVWFKNDKDIELSEHFLVKMEQSKYVSLTIQGVTAEDSGKYSINVKNK |
| | | YGGEKIDVTVSVYKHGEKIPDISPPQQAKPKLIPASTSSD |
| 16 | Mouse ALBU | MKWVTFLLLLFVSGSAFSRGVFRREAHKSEIAHRYNDLGEQHFKGLVLIAFSQYLQK |
| | (albumin) | CSYDEHAKLVQEVTDFAKTCVADESAANCDKSLHTLFGDKLCAIPNLRENYGELAD |
| | | CCTKQEPERNECFLQHKDDNPSLPPFERPEAEAMCTSFKENPTTFMGHYLHEVARRH |
| | | PYFYAPELLYYAEQYNEILTQCCAEADKESCLTPKLDGVKEKALVSSVRQRMKCSS |
| | | MQKFGERAFKAWAVARLSQTFPNADFAEITKLATDLTKVNKECCHGDLLECADDR |
| | | AELAKYMCENQATISSKLQTCCDKPLLKKAHCLSEVEHDTMPADLPAIAADFVEDQ |
| | | EVCKNYAEAKDVFLGTFLYEYSRRHPDYSVSLLLRLAKKYEATLEKCCAEANPPAC |
| | | YGTVLAEFQPLVEEPKNLVKTNCDLYEKLGEYGFQNAILVRYTQKAPQVSTPTLVEA |
| | | ARNLGRVGTKCCTLPEDQRLPCVEDYLSAILNRVCLLHEKTPVSEHVTKCCSGSLVE |
| | | RRPCFSALTVDETYVPKEFKAETFTFHSDICTLPEKEKQIKKQTALAELVKHKPKATA |
| | | EQLKTVMDDFAQFLDTCCKAADKDTCFSTEGPNLVTRCKDALA |
| 17 | Mouse ENOB | MAMQKIFAREILDSRGNPTVEVDLHTAKGRFRAAVPSGASTGIYEALELRDGDKARY |
| | (beta-enolase) | LGKGVLKAVEHINKTLGPALLEKKLSVVDQEKVDKFMIELDGTENKSKFGANAILG |
| | | VSLAVCKAGAAEKGVPLYRHIADLAGNPDLVLPVPAFNVINGGSHAGNKLAMQEF |
| | | MILPVGASSFKEAMRIGAEVYHHLKGVIKAKYGKDATNVGDEGGFAPNILENNEAL |
| | | ELLKTAIQAAGYPDKVVIGMDVAASEFYRNGKYDLDFKSPDDPARHISGEKLGELYK |
| | | NFIQNYPVVSIEDPFDQDDWATWTSFLSGVDIQIVGDDLTVTNPKRIAQAVEKKACN |
| | | CLLLKVNQIGSVTESIQACKLAQSNGWGVMVSHRSGETEDTFIADLVVGLCTGQIKT |
| | | GAPCRSERLAKYNQLMRIEEALGDKAVFAGRKFRNPKAK |
| 18 | Mouse MLRS | MAPKKAKRRAGAEGSSNVFSMFDQTQIQEFKEAFTVIDQNRDGIIDKEDLRDTFAAM |
| | (myosin | GRLNVKNEELDAMMKEASGPINFTVFLTMFGEKLKGADPEDVITGAFKVLDPEGKG |
| | regulatory light | TIKKQFLEELLTTQCDRFSQEEIKNMWAAFPPDVGGNVDYKNICYVITHGDAKDQE |
| | chain 2) | |
| 19 | Mouse KPYR | MSVQENELPQQLWPWIFKSQKDLAKSALSGAPGGPAGYLRRASVAQLTQELGTAFF |
| | (pyruvate kinase | QQQQLPAAMADTFLEHLCLLDIDSEPVAARSTSIIATIGPASRSVDRLKEMIKAGMNI |
| | PKLR) | ARLNFSHGSHEYHAESIANIREAAESFATSPLSYRPVAIALDTKGPEIRTGVLQGGPES |
| | | EVEIVKGSQVLVTVDPKFRTRGDAKTVWVDYHNITQVVAVGGRIYIDDGLISLVVRK |
| | | IGPEGLVTEVEHGGFLGNRKGVNLPNAEVDLPGLSEQDLLDLRFGVEHYVDIIFASFV |
| | | RKASDVVAVRDALGPEGRGIKIISKIENHEGVKKFDEILEVSDGIMMARGDLGIEIPAE |
| | | KVFLAQKMMIGRCNLAGKPVVCATQMLESMITKARPTRAETSDVANAVLDGADCI |
| | | MLSGETAKGSFPVEAVKMQHAIAREAEAAVYHRQLFEELRRAAPLSRDPTEVTAIGA |
| | | VEASFKCCAAAIIVLTKTGRSAQLLSRYRPRAAVIAVTRSAQAARQVHLSRGVFPLLY |
| | | REPPEAVWADDVDRRVQFGIESGKLRGFLRVGDLVIVVTGWRPGSGYTNIMRVLTIS |
| 20 | Mouse CAH3 | MAKEWGYASHNGPDHWHELYPIAKGDNQSPIELHTKDIKHDPSLQPWSASYDPGSA |
| | (carbonic | KTILNNGKTCRVVFDDTYDRSMLRGGPLSGPYRLRQFHLHWGSSDDHGSEHTVDGV |
| | anhydrase 3) | KYAAELHLVHWNPKYNTFGEALKQPDGIAVVGIFLKIGREKGEFQILLDALDKIKTK |
| | | GKEAPFTHFDPSCLFPACRDYWTYHGSFTTPPCEECIVWLLLKEPMTVSSDQMAKLR |
| | | SLFSSAENEPPVPLVGNWRPPQPVKGRVVRASFK |
| 21 | Mouse LDHA | MATLKDQLIVNLLKEEQAPQNKITVVGVGAVGMACAISILMKDLADELALVDVMED |
| | (L-lactate | KLKGEMMDLQHGSLFLKTPKIVSSKDYCVTANSKLVIITAGARQQEGESRLNLVQRN |
| | dehydrogenase | VNIFKFIIPNIVKYSPHCKLLIVSNPVDILTYVAWKISGFPKNRVIGSGCNLDSARFRYL |
| | A) | MGERLGVHALSCHGWVLGEHGDSSVPVWSGVNVAGVSLKSLNPELGTDADKEQW |
| | | KEVHKQVVDSAYEVIKLKGYTSWAIGLSVADLAESIMKNLRRVHPISTMIKGLYGIN |
| | | EDVFLSVPCILGQNGISDVVKVTLTPEEEARLKKSADTLWGIQKELQF |
| 22 | Mouse | MSSDAEMAVFGEAAPYLRKSEKERIEAQNRPFDAKTSVFVAEPKESFVKGTIQSKDA |
| | G3UW82 | GKVTVKTEAGATLTVKEDQIFPMNPPKYDKIEDMAMMTHLHEPAVLYNLKERYAA |
| | (myosin, heavy | WMIYTYSGLFCVTVNPYKWLPVYNPEVVAAYRGKKRQEAPPHIFSISDNAYQFMLT |
| | polypeptide 2) | DRENQSILITGESGAGKTVNTKRVIQYFATIAVTGDKKKEEATSGKMQGTLEDQIISA |
| | | NPLLEAFGNAKTVRNDNSSRFGKFIRIHFGTTGKLASADIETYLLEKSRVTFQLKAER |
| | | SYHIFYQITSNKKPELIEMLLITTNPYDYPFVSQGEISVASIDDQEELMATDSAIDILGFT |
| | | NDEKVSIYKLTGAVMHYGNMKFKQKQREEQAEPDGTEVADKAAYLQGLNSADLLK |
| | | ALCYPRVKVGNEYVTKGQTVEQVTNAVGALAKAMYEKMFLWMVTRINQQLDTKQ |
| | | PRQYFIGVLDIAGFEIFDFNSLEQLCINFTNEKLQQFFNHHMFVLEQEEYKKEGIEWTF |
| | | IDFGMDLAACIELIEKPMGIFSILEEECMFPKATDTSFKNKLYEQHLGKSANFQKPKV |
| | | VKGKAEAHFSLIHYAGTVDYNITGWLDKNKDPLNETVVGLYQKSSVKTLAYLFSGA |
| | | QTAEAEASSGGAAKKGAKKKGSSFQTVSALFRENLNKLMTNLRSTHPHFVRCIIPNE |
| | | TKTPGAMEHELVLHQLRCNGVLEGIRICRKGFPSRILYADFKQRYKVLNASAIPEGQY |
| | | IDSKKASEKLLGSIDIDHTQYKFGHTKVFFKAGLLGLLEEMRDDKLAQLITRTQAMC |
| | | RGFLARVEYQKMVERRESIFCIQYNIRAFMNVKHWPWMKLFFKIKPLLKSAETEKE |
| | | MATMKEEFQKTKDDLAKSEAKRKELEEKMVSLLKEKNDLQLQVQAEAEGLADAEE |
| | | RCDQLIKTKIQLEAKIKEVTERAEDEEEINAELTAKKRKLEDECSELKKDIDDLELTLA |
| | | KVEKEKHATENKVKNLTEEMAGLDETIAKLTKEKKALQEAHQQTLDDLQAEEDKV |
| | | NTLTKAKIKLEQQVDDLEGSLEQEKKLRMDLERAKRKLEGDLKLAQESIMDIENEKQ |
| | | QLDERLKKKEFEMSNLQSKIEDEQAIGIQLQKKIKELQARIEELEEEIEAERASRAKAE |
| | | KQRSDLSRELEEISERLEEAGGATSAQIEMNKKREAEFQKMRRDLEEATLQHEATAA |
| | | TLRKKHADSVAELGEQIDNLQRVKQKLEKEKSEMKMEIDDLASNVETVSKAKGNLE |
| | | KMCRTLEDQVSELKSKEEEQQRLINDLTTQRGRLQTESGEFSRQLDEKEALVSQLSR |
| | | GKQAFTQQIEELKRQLEEEVKAKNALAHALQSSRHDCDLLREQYEEEQESKAELQR |
| | | ALSKANSEVAQWRTKYETDAIQRTEELEEAKKKLAQRLQAAEEHVEAVNAKCASLE |
| | | KTKQRLQNEVEDLMLDVERTNAACAALDKKQRNFDKILAEWKQKYEETHAELEAS |
| | | QKEARSLGTELFKMKNAYEESLDQLETLKRENKNLQQEISDLTEQIAEGGKRIHELEK |
| | | IKKQVEQEKCELQAALEEAEASLEHEEGKILRIQLELNQVKSEIDRKIAEKDEEIDQLK |
| | | RNHIRVVESMQSTLDAEIRSRNDAIRIKKKMEGDLNEMEIQLNHSNRMAAEALRNYR |
| | | NTQGILKDTQLHLDDALRGQEDLKEQLAMVERRANLLQAEIEELRATLEQTERSRKI |
| | | AEQELLDASERVQLLHTQNTSLINTKKKLETDISQIQGEMEDIVQEARNAEEKAKKAI |
| | | TDAAMMAEELKKEQDTSAHLERMKKNMEQTVKDLQLRLDEAEQLALKGGKKQIQ |
| | | KLEARVRELEGEVESEQKRNAEAVKGLRKHERRVKELTYQTEEDRKNILRLQDLVD |
| | | KLQAKVKSYKRQAEEAEEQSNTNLSKFRKIQHELEEAEERADIAESQVNKLRVKSRE |
| | | VHTKIISEE |
| 23 | Mouse Q6P6L5 | MPEPTKKEENEVSAPAPPPEEPNKEKEAGTAPAKEWSLGESPAGGEEQDKQNANSQL |
| | (myosin-binding | STLFVEKPQTGSVKVGANITFVAKVRAEDLLRKPTVKWFKGKWMDLASKAGKHLQ |
| | protein C) | LKETFERQTRVYTFEMQIIKAKENYAGNYRCEVTYKDKFDSCSFDLEVHESTGTTPNI |
| | | DIRSAFKRSGEGQEDAGELDFSGLLKRREVKQQEEEPEIDVWELLKNANPNEYEKIAF |
| | | QYGITDLRGMLKRLKRMRRVEKKSAAFAKILDPAYQVDKGGKVRFVVELADPKLE |
| | | VKWFKNGQEIRPSTKYIFEHKGNERIMFINNCSLTDDSEYYVTAGDEKCSTELFVREP |
| | | PIMVTKQLEDMNAYCGERVEMEVEVSEDDANVKWFKNGEEIFPGPKSRYKIKVEGK |
| | | KHTLIIEGATKADSAEYSAMTTGGQSSAKLSVDLRPLKITTPLTDQTVKLGKEVCLKC |
| | | EISENVPGKWTKNGLPVQEGERLKVVHKGRIHKLVIANALIEDEGEYVFTPDAYNVP |
| | | LSAKVHVIDPPKIILDGLDADNTVTVIAGSKLRLEIPVTGEPPPKAIWSRADKAIMEGS |
| | | GRIRAESYPDSSTLVIDVAERDDSGVYNINLKNEAGEAHASIKIKVVDIPDPPVAPNVT |
| | | EVGDDWCIMNWEPPAYDGGSPILGYFIERKKKQSSRWMRLNFDLCKETTFEPKKMIE |
| | | GVAYEVRIFAVNAIGISKPSMPSKPFVPLAVTSPPTLLAVDSVTDSSVTMKWRPPDQI |
| | | GAAGLDGYVLEYCFEGTEDWIVANTDLIDKTKFTINGLPTDAKIFVRVKAINAAGAS |
| | | EPKYYSQPILVKEIIEPPKIRIPRHLKQTYIRRVGEAVNLVIPFQGKPRPELTWKKDGEE |
| | | IDKNQINIRNSETDTIIFIRKAERSHSGKYDLQVKVDKYVENASIDIQIVDRPGPPQTVT |
| | | IEDVWGENVALTWTPPKDDGNAAITGYTIQKADKKSMEWFTVIEHYHRTNATITEL |
| | | VIGNEYYFRVFAENMCGLSEDATMTKESAVIAKDGKIYKNPVYEDFNFTEAPMFTQP |
| | | LVNTYAIAGYNATLNCSVRGNPKPKITWMKNKVAIVDDPRYRMFSNQGVCTLEIRK |
| | | PSPYDGGTYCCKAVNDLGTVEIECKLEVKVVAQ |
| 24 | Mouse PGM1 | MVKIVTVKTQAYPDQKPGTSGLRKRVKVFQSNANYAENFIQSIVSTVEPALRQEATL |
| | (phosphogluco- | VVGGDGRFYMTEAIQLIVRIAAANGIGRLVIGQNGILSTPAVSCIIRKIKAIGGIILTASH |
| | mutase-1) | NPGGPNGDFGIKFNISNGGPAPEAITDKIFQISKTIEEYAICPDLKVDLGVLGKQQFDLE |
| | | NKFKPFTVEIVDSVEAYATMLRNIFDFNALKELLSGPNRLKIRIDAMHGVVGPYVKKI |
| | | LCEELGAPANSAVNCVPLEDFGGHHPDPNLTYAADLVETMKSGEHDFGAAFDGDG |
| | | DRNMILGKHGFFVNPSDSVAVIAANIFSIPYFQQTGVRGFARSMPTSGALDRVANAT |
| | | KIALYETPTGWKFFGNLMDASKLSLCGEESFGTGSDHIREKDGLWAVLAWLSILATR |
| | | KQSVEDILKDHWQKFGRNFFTRYDYEEVEAEGANKMMKDLEALMLDRSFVGKQFS |
| | | ANDKVYTVEKADNFEYSDPVDGSISKNQGLRLIFADGSRIIFRLSGTGSAGATIRLYID |
| | | SYEKDVAKINQDPQVMLAPLISIALKVSQLQERTGRTAPTVIT |
| 25 | Mouse TPM2 | MDAIKKKMQMLKLDKENAIDRAEQAEADKKQAEDRCKQLEEEQQALQKKLKGTE |
| | (tropomyosin | DEVEKYSESVKDAQEKLEQAEKKATDAEADVASLNRRIQLVEEELDRAQERLATAL |
| | beta chain) | QKLEEAEKAADESERGMKVIENRAMKDEEKMELQEMQLKEAKHIAEDSDRKYEEV |
| | | ARKLVILEGELERSEERAEVAESKCGDLEEELKIVTNNLKSLEAQADKYSTKEDKYE |
| | | EEIKLLEEKLKEAETRAEFAERSVAKLEKTIDDLEDEVYAQKMKYKAISEELDNALN |
| | | DITSL |
| 26 | Mouse TPIS | MAPTRKFFVGGNWKMNGRKKCLGELICTLNAANVPAGTEVVCAPPTAYIDFARQKL |
| | (triosephosphate | DPKIAVAAQNCYKVTNGAFTGEISPGMIKDLGATWVVLGHSERRHVFGESDELIGQK |
| | isomerase) | VSHALAEGLGVIACIGEKLDEREAGITEKVVFEQTKVIADNVKDWSKVVLAYEPVW |
| | | AIGTGKTATPQQAQEVHEKLRGWLKSNVNDGVAQSTRIIYGGSVTGATCKELASQP |
| | | DVDGFLVGGASLKPEFVDIINAKQ |
| 27 | Mouse PGAM2 | MTTHRLVMVRHGESLWNQENRFCGWFDAELSEKGAEEAKRGATAIKDAKIEFDICY |
| | (phosphogly- | TSVLKRAIRTLWTILDVTDQMWVPVVRTWRLNERHYGGLTGLNKAETAAKHGEEQ |
| | cerate | VKIWRRSFDTPPPPMDEKHNYYTSISKDRRYAGLKPEELPTCESLKDTIARALPFWNE |
| | mutase 2) | EIAPKIKAGQRVLIAAHGNSLRGIVKHLEGMSDQAIMELNLPTGIPIVYELDQNLKPT |
| | | KPMRFLGDEETVRKAMEAVAAQGKAK |
| 28 | Mouse SRCA | MKALLLLCCFLASLLLSGQAEVEDASEEAPLRDRSHIDKTLMLNEDKPADDYSAVLQ |
| | (sarcalumenin) | RLRKIYHTSIKPLEQSYKYNELRQHEITDGEITSKPMVLFLGPWSVGKSTMINYLLGL |
| | | EDTRYQLYTGAEPTTSEFTVLMHGPKLKTIEGIVMAADSARSFSPLEKFGQNFLEKLI |
| | | GIEVPHKLLERVTFVDTPGIIENRKQQERGYPFNDVCQWFIDRADLIFVVFDPTKLDV |
| | | GLELEMLFRQLKGRESQIRIILNKADNLATQMLMRVYGALFWSLAPLINVTEPPRVY |
| | | VSSFWPQDYKPDTHRELFLKEEISLLEDLNQVIENRLENKIAFIRQHAIRVRIHALLVD |
| | | RYLQTYKDKMTFFSDGELVFKDIVEDPDKFYIFKTILAKTNVSKFDLPNREAYKDFFG |
| | | INPISNFKLLSQQCSYMGGCFLEKIERAITQELPSLLGSIGLGKNPGAPNCDKTGCGET |
| | | PKNRYKKH |
| 29 | Mouse PGK 1 | MSLSNKLTLDKLDVKGKRVVMRVDFNVPMKNNQITNNQRIKAAVPSIKFCLDNGAK |
| | (phosphoglycerate | SVVLMSHLGRPDGVPMPDKYSLEPVAAELKSLLGKDVLFLKDCVGPEVENACANPA |
| | kinase 1) | AGTVILLENLRFHVEEEGKGKDASGNKVKAEPAKIDAFRASLSKLGDVYVNDAFGT |
| | | AHRAHSSMVGVNLPQKAGGFLMKKELNYFAKALESPERPFLAILGGAKVADKIQLIN |
| | | NMLDKVNEMIIGGGMAFTFLKVLNNMEIGTSLYDEEGAKIVKDLMSKAEKNGVKIT |
| | | LPVDFVTADKFDENAKTGQATVASGIPAGWMGLDCGTESSKKYAEAVGRAKQIVW |
| | | NGPVGVFEWEAFARGTKSLMDEVVKATSRGCITIIGGGDTATCCAKWNTEDKVSHV |
| | | STGGGASLELLEGKVLPGVDALSNV |
| 30 | Mouse PLEC | MVAGMLMPLDRLRAIYEVLFREGVMVAKKDRRPRSLHPHVPGVTNLQVMRAMAS |
| | (plectin) | LKARGLVRETFAWCHFYWYLTNEGIDHLRQYLHLPPEIVPASLQRVRRPVAMVIPAR |
| | | RRSPHVQTMQGPLGCPPKRGPLPAEDPAREERQVYRRKEREEGAPETPVVSATTVGT |
| | | LARPGPEPAPATDERDRVQKKTFTKWVNKHLIKHWRAEAQRHISDLYEDLRDGHNL |
| | | ISLLEVLSGDSLPREKGRMRFHKLQNVQIALDYLRHRQVKLVNIRNDDIADGNPKLT |
| | | LGLIWTIILHFQISDIQVSGQSEDMTAKEKLLLWSQRMVEGYQGLRCDNFTTSWRDG |
| | | RLFNAIIHRHKPMLIDMNKVYRQTNLENLDQAFSVAERDLGVTRLLDPEDVDVPQPD |
| | | EKSIITYVSSLYDAMPRVPGAQDGVRANELQLRWQEYRELVLLLLQWIRHHTAAFEE |
| | | RKFPSSFEEIEILWCQFLKFKETELPAKEADKNRSKVIYQSLEGAVQAGQLKIPPGYHP |
| | | LDVEKEWGKLHVAILEREKQLRSEFERLECLQRIVSKLQMEAGLCEEQLNQADALLQ |
| | | SDIRLLASGKVAQRAGEVERDLDKADGMIRLLFNDVQTLKDGRHPQGEQMYRRVY |
| | | RLHERLVAIRTEYNLRLKAGVGAPVTQVTLQSTQRRPELEDSTLRYLQDLLAWVEEN |
| | | QRRIDSAEWGVDLPSVEAQLGSHRGMHQSIEEFRAKIERARNDESQLSPATRGAYRD |
| | | CLGRLDLQYAKLLNSSKARLRSLESLHGFVAAATKELMWLNEKEEEEVGFDWSDRN |
| | | TNMAAKKESYSALMRELEMKEKKIKEIQNTGDRLLREDHPARPTVESFQAALQTQW |
| | | SWMLQLCCCIEAHLKENTAYFQFFSDVREAEEQLQKLQETLRRKYSCDRTITVTRLE |
| | | DLLQDAQDEKEQLNEYKGHLSGLAKRAKAIVQLKPRNPAHPVRGHVPLIAVCDYKQ |
| | | VEVTVHKGDQCQLVGPAQPSHWKVLSGSSSEAAVPSVCFLVPPPNQEAQEAVARLE |
| | | AQHQALVTLWHQLHVDMKSLLAWQSLSRDIQLIRSWSLVTFRTLKPEEQRQALRNL |
| | | ELHYQAFLRDSQDAGGFGPEDRLVAEREYGSCSRHYQQLLQSLEQGEQEESRCQRCI |
| | | SELKDIRLQLEACETRTVHRLRLPLDKDPARECAQRIAEQQKAQAEVEGLGKGVARL |
| | | SAEAEKVLALPEPSPAAPTLRSELELTLGKLEQVRSLSAIYLEKLKTISLVIRSTQGAEE |
| | | VLKTHEEQLKEAQAVPATLQELEATKASLKKLRAQAEAQQPVFNTLRDELRGAQEV |
| | | GERLQQRHGERDVEVERWRERVTQLLERWQAVLAQTDVRQRELEQLGRQLRYYRE |
| | | SADPLSAWLQDAKRRQEQIQAVPIANCQAAREQLRQEKALLEEIERHGEKVEECQKF |
| | | AKQYINAIKDYELQLITYKAQLEPVASPAKKPKVQSGSESVIQEYVDLRTRYSELTTL |
| | | TSQYIKFISETLRRMEEEERLAEQQRAEERERLAEVEAALEKQRQLAEAHAQAKAQA |
| | | ELEAQELQRRMQEEVARREEAAVDAQQQKRSIQEELQHLRQSSEAEIQAKAQQVEA |
| | | AERSRMRIEEEIRVVRLQLETTERQRGGAEGELQALRARAEEAEAQKRQAQEEAERL |
| | | RRQVQDESQRKRQAEAELALRVKAEAEAAREKQRALQALDELRLQAEEAERRLRQ |
| | | AEAERARQVQVALETAQRSAEVELQSKRASFAEKTAQLERTLQEEHVTVAQLREEA |
| | | ERRAQQQAEAERAREEAERELERWQLKANEALRLRLQAEEVAQQKSLAQADAEKQ |
| | | KEEAEREARRRGKAEEQAVRQRELAEQELEKQRQLAEGTAQQRLAAEQELIRLRAE |
| | | TEQGEQQRQLLEEELARLQHEATAATQKRQELEAELAKVRAEMEVLLASKARAEEE |
| | | SRSTSEKSKQRLEAEAGRFRELAEEAARLRALAEEAKRQRQLAEEDAARQRAEAER |
| | | VLTEKLAAISEATRLKTEAEIALKEKEAENERLRRLAEDEAFQRRRLEEQAALHKADI |
| | | EERLAQLRKASESELERQKGLVEDTLRQRRQVEEEIMALKVSFEKAAAGKAELELEL |
| | | GRIRSNAEDTMRSKEQAELEAARQRQLAAEEEQRRREAEERVQRSLAAEEEAARQR |
| | | KVALEEVERLKAKVEEARRLRERAEQESARQLQLAQEAAQKRLQAEEKAHAFVVQ |
| | | QREEELQQTLQQEQNMLDRLRSEAEAARRAAEEAEEAREQAEREAAQSRKQVEEAE |
| | | RLKQSAEEQAQAQAQAQAAAEKLRKEAEQEAARRAQAEQAALKQKQAADAEMEK |
| | | HKKFAEQTLRQKAQVEQELTTLRLQLEETDHQKSILDEELQRLKAEVTEAARQRSQV |
| | | EEELFSVRVQMEELGKLKARIEAENRALILRDKDNTQRFLEEEAEKMKQVAEEAARL |
| | | SVAAQEAARLRQLAEEDLAQQRALAEKMLKEKMQAVQEATRLKAEAELLQQQKEL |
| | | AQEQARRLQEDKEQMAQQLVEETQGFQRTLEAERQRQLEMSAEAERLKLRMAEMS |
| | | RAQARAEEDAQRFRKQAEEIGEKLHRTELATQEKVTLVQTLEIQRQQSDHDAERLRE |
| | | AIAELEREKEKLKQEAKLLQLKSEEMQTVQQEQILQETQALQKSFLSEKDSLLQRERF |
| | | IEQEKAKLEQLFQDEVAKAKQLREEQQRQQQQMEQEKQELMASMEEARRRQREAE |
| | | EGVRRKQEELQHLEQQRQQQEKLLAEENQRLRERLQRLEEEHRAALAHSEIATTQA |
| | | ASTKALPNGRDAPDGPSVEAEPEYTFEGLRQKVPAQQLQEAGILSQEELQRLAQGHT |
| | | TVAELTQREDVYRYLKGRSSIAGLLLKPTNEKLSVYTALQRQLLSPGTALILLEAQAA |
| | | SGFLLDPVRNRRLTVNEAVKEGVVGPELHHKLLSAERAVTGYKDPYTGEQISLFQA |
| | | MKKDLIVRDHGVRLLEAQIATGGIIDPVHSHRVPVDVAYKRGYFDEEMNRILSDPSD |
| | | DTKGFFDPNTHENLTYLQLLERCVEDPETGLRLLPLTDKAAKGGELVYTDTEARDVF |
| | | EKATVSAPFGKFQGRTVTIWEIINSEYFTAEQRRDLLQQFRTGHITVEKIIKIVITVVEE |
| | | HERKGQLCFEGLRALVPAAELLDSGVISHELYQQLQRGERSVREVAEADSVRQALR |
| | | GTNVIAGVWLEEAGQKLSIYEALKKDLLQPEVAVALLEAQAGTGHIIDPATSARLTV |
| | | DEAVRAGLVGPELHEKLLSAEKAVTGYRDPYSGQSVSLFQALKKGLIPREQGLRLLD |
| | | AQLSTGGIVDPSKSHRVPLDVAYARGYLDKETNRALTSPRDDARVYHDPSTQEPVTY |
| | | SQLQQRCRSDQLTGLSLLPLSEKAVRARQEEVYSELQARETLEQAKVEVPVGSFKGR |
| | | AMTVWELISSEYFTEEQRQELLRQFRTGKVTVEKVIKIVITIVEEVETRRQERLSFSGL |
| | | RAPVPASELLDAKILSRAQFDQLKDGKTSVKELSEVGSVRTLLQGSGCLAGIYLEDSK |
| | | EKVTIYEAMRRGLLRPSTATLLLEAQAATGFLVDPVRNQRLYVHEAVKAGVVGPEL |
| | | HEKLLSAEKAVTGYKDPYSGNTISLFQAMKKGLVLRDHAIRLLEAQVATGGIIDPVH |
| | | SHRLPVDVAYQRGYFDEEMNRVLADPSDDTKGFFDPNTHENLTYLQLLERCVEDPE |
| | | TGLRLLPLKGAEKTEVVETTQVYTEEETRRAFEETQIDIPGGGSHGGSSMSLWEVMQ |
| | | SNMIPEDQRARLMADFQAGRVTKERMIIIIIEIIEKTEIIRQQNLASYDYVRRRLTAEDL |
| | | YEARIISLETYNLFREGTKNLREVLEMESAWRYLYGTGAVAGVYLPGSRQTLTIYQA |
| | | LKKGLLSAEVARLLLEAQAATGFLLDPVKGERLTVDEAVRKGLVGPELHDRLLSAE |
| | | RAVTGYRDPYTEQTISLFQAMKKELIPAEEALRLLDAQLATGGIVDPRLGFHLPLEVA |
| | | YQRGYLNKDTHDQLSEPSEVRSYVDPSTDERLSYTQLLKRCRRDDPSGQMLLLLSDA |
| | | RKLTFRGLRKQITVEELVRSQVMDEATALQLQEGLTSIEEVTKNLQKFLEGTSCIAGV |
| | | FVDATKERLSVYQAMKKGIIRPGTAFELLEAQAATGYVIDPIKGLKLTVEEAVRMGI |
| | | VGPEFKDKLLSAERAVTGYKDPYSGKLISLFQAMKKGLILKDHGIRLLEAQIATGGII |
| | | DPEESHRLPVEVAYKRGLFDEEMNEILTDPSDDTKGFFDPNTEENLTYLQLMERCITD |
| | | PQTGLCLLPLKEKKRERKTSSKSSVRKRRVVIVDPETGKEMSVYEAYRKGLIDHQTY |
| | | LELSEQECEWEEITISSSDGVVKSMIIDRRSGRQYDIDDAITKNLIDRSALDQYRAGTL |
| | | SITEFADMLSGNAGGFRSRSSSVGSSSSYPISSAGPRTQLASWSDPTEETGPVAGILDT |
| | | ETLEKVSITEAMHRNLVDNITGQRLLEAQACTGGIIDPSTGERFPVTEAVNKGLVDKI |
| | | MVDRINLAQKAFCGFEDPRTKTKMSAAQALKKGWLYYEAGQRFLEVQYLTGGLIEP |
| | | DTPGRVSLDEALQRGTVDARTAQKLRDVSAYSKYLTCPKTKLKISYKDALDRSMVE |
| | | EGTGLRLLEAAAQSSKGYYSPYSVSGSGSTAGSRTGSRTGSRAGSRRGSFDATGSGFS |
| | | MTFSSSSYSSSGYGRRYASGPSASLGGPESAVA |
| 31 | Mouse ACTN2 | MNQIEPGVQYNYVYDEDEYMIQEEEWDRDLLLDPAWEKQQRKTFTAWCNSHLRKA |
| | (alpha-actinin-2) | GTQIENIEEDFRNGLKLMLLLEVISGERLPKPDRGKMRFHKIANVNKALDYIASKGVK |
| | | LVSIGAEEIVDGNVKMTLGMIWTIILRFAIQDISVEETSAKEGLLLWCQRKTAPYRNV |
| | | NIQNFHTSWKDGLGLCALIHRHRPDLIDYSKLNKDDPIGNINLAMEIAEKHLDIPKML |
| | | DAEDIVNTPKPDERAIMTYVSCFYHAFAGAEQAETAANRICKVLAVNQENERLMEE |
| | | YERLASELLEWIRRTIPWLENRTPEKTMQAMQKKLEDFRDYRRKHKPPKVQEKCQL |
| | | EINFNTLQTKLRISNRPAFMPSEGKMVSDIAGAWQRLEQAEKGYEEWLLNEIRRLER |
| | | LEHLAEKFRQKASTHETWAYGKEQILLQKDYESASLTEVRALLRKHEAFESDLAAH |
| | | QDRVEQIAAIAQELNELDYHDAVNVNDRCQKICDQWDRLGTLTQKRREALERTEKL |
| | | LETIDQLHLEFAKRAAPFNNWMEGAMEDLQDMFIVHSIEEIQSLITAHEQFKATLPEA |
| | | DGERQSILAIQNEVEKVIQSYSIRISSSNPYSTVTMDELRNKWDKVKQLVPVRDQSLQ |
| | | EELARQHANERLRRQFAAQANAIGPWIQNKMEEIARSSIQITGALEDQMNQLKQYEH |
| | | NIINYKNNIDKLEGDHQLIQEALVFDNKHTNYTMEHIRVGWELLLTTIARTINEVETQI |
| | | LTRDAKGITQEQMNEFRASFNHFDRRKNGLMDHEDFRACLISMGYDLGEAEFARIM |
| | | TLVDPNGQGTVTFQSFIDFMTRETADTDTAEQVIASFRILASDKPYILAEELRRELPPD |
| | | QAQYCIKRMPPYSGPGSVPGALDYTAFSSALYGESDL |
| 32 | Mouse PFKAM | MTHEEHHAAKTLGIGKAIAVLTSGGDAQGMNAAVRAVVRVGIFTGARVFFVHEGY |
| | (ATP-dependent | QGLVDGGEHIREATWESVSMMLQLGGTVIGSARCKDFREREGRLRAAHNLVKRGIT |
| | 6- | NLCVIGGDGSLTGADTFRSEWSDLLNDLQKDGKITAEEATKSSYLNIVGLVGSIDNDF |
| | phosphofru- | CGTDMTIGTDSALHRIVEIVDAITTTAQSHQRTFVLEVMGRHCGYLALVTSLSCGAD |
| | nctokiase) | WVFIPECPPDDDWEEHLCRRLSETRTRGSRLNIIIVAEGAIDKNGKPITSEDIKNLVVK |
| | | RLGYDTRVTVLGHVQRGGTPSAFDRILGSRMGVEAVMALLEGTPDTPACVVSLSGN |
| | | QAVRLPLMECVQVTKDVTKAMDEKRFDEAIKLRGRSFMNNWEVYKLLAHVRPPVS |
| | | KGGLHTVAVMNVGAPAAGMNAAVRSTVRIGLIQGNRVLVVHDGFEGLAKGQIEEA |
| | | GWSYVGGWTGQGGSKLGTKRTLPKKNLEQISANITKFNIQGLVIIGGFEAYTGGLEL |
| | | MEGRKQFDELCIPFVVIPATVSNNVPGSDFSIGADTALNTICTTCDRIKQSAAGTKRR |
| | | VFIIETMGGYCGYLATMAGLAAGADAAYIFEEPFTIRDLQVNVEHLVQKMKTTVKR |
| | | GLVLRNEKCNENYTTDFIFNLYSEEGKGIFDSRKNVLGHMQQGGSPTPFDRNFATKM |
| | | GAKAMNWMSGKIKESYRNGRIFANTPDSGCVLGMRKRALVFQPVTELKDQTDFEH |
| | | RIPKEQWWLKLRPILKILAKYEIDLDTSDHAHLEHISRKRSGEAAV |
| 33 | Mouse MDHM | MLSALARPAGAALRRSFSTSAQNNAKVAVLGASGGIGQPLSLLLKNSPLVSRLTLYDI |
| | (malate | AHTPGVAADLSHIETRANVKGYLGPEQLPDCLKGCDVVVIPAGVPRKPGMTRDDLF |
| | dehydrogenase) | NTNATIVATLTAACAQHCPEAMVCIIANPVNSTIPITAEVFKKHGVYNPNKIFGVTTL |
| | | DIVRANTFVAELKGLDPARVNVPVIGGHAGKTIIPLISQCTPKVDFPQDQLATLTGRIQ |
| | | EAGTEVVKAKAGAGSATLSMAYAGARFVFSLVDAMNGKEGVVECSFVQSKETECT |
| | | YFSTPLLLGKKGLEKNLGIGKITPFEEKMIAEAIPELKASIKKGEDFVKNMK |
| 34 | Mouse MYG | MGLSDGEWQLVLNVWGKVEADLAGHGQEVLIGLFKTHPETLDKFDKFKNLKSEED |
| | (myoglobin) | MKGSEDLKKHGCTVLTALGTILKKKGQHAAEIQPLAQSHATKHKIPVKYLEFISEIIIE |
| | | VLKKRHSGDFGADAQGAMSKALELFRNDIAAKYKELGFQG |
| 35 | Mouse HBBB1 | MVHLTDAEKAAVSCLWGKVNSDEVGGEALGRLLVVYPWTQRYFDSFGDLSSASAI |
| | (hemoglobin | MGNAKVKAHGKKVITAFNDGLNHLDSLKGTFASLSELHCDKLHVDPENFRLLGNMI |
| | subunit beta-1) | VIVLGHHLGKDFTPAAQAAFQKVVAGVATALAHKYH |
| 36 | Mouse FLNC | MMNNSNYSDASGLGLVDEADEMPSTEKDLAEDAPWKKIQQNTFTRWCNEHLKCVG |
| | (filamin-C) | KRLTDLQRDLSDGLRLIALLEVLSQKRMYRKFHPRPNFRQMKLENVSVALEFLEREH |
| | | IKLVSIDSKAIVDGNLKLILGLIWTLILHYSISMPMWEDEDDEDARKQTPKQRLLGWI |
| | | QNKVPQLPITNFNRDWQDGKALGALVDNCAPGLCPDWEAWDPNQPVQNAREAMQ |
| | | QADDWLGVPQVIAPEEIVDPNVDEHSVMTYLSQFPKAKLKPGAPVRSKQLNPKKAIA |
| | | YGPGIEPQGNTVLQPAHFTVQTVDAGVGEVLVYIEDPEGHTEEAKVVPNNDKDRTY |
| | | AVSYVPKVAGLHKVTVLFAGQNIERSPFEVNVGMALGDANKVSARGPGLEPVGNV |
| | | ANKPTYFDIYTAGAGTGDVAVVIVDPQGRRDTVEVALEDKGDNTFRCTYRPVMEGP |
| | | HTVHVAFAGAPITRSPFPVHVAEACNPNACRASGRGLQPKGVRVKEVADFKVFTKG |
| | | AGSGELKVTVKGPKGTEEPVKVREAGDGVFECEYYPVVPGKYVVTITWGGYAIPRS |
| | | PFEVQVSPEAGAQKVRAWGPGLETGQVGKSADFVVEAIGTEVGTLGFSIEGPSQAKI |
| | | ECDDKGDGSCDVRYWPTEPGEYAVHVICDDEDIRDSPFIAHIQPAPPDCFPDKVKAF |
| | | GPGLEPTGCIVDRPAEFTIDARAAGKGDLKLYAQDADGCPIDIKVIPNGDGTFRCSYV |
| | | PTKPIKHTIIVSWGGVNVPKSPFRVNVGEGSHPERVKVYGPGVEKTGLKANEPTYFT |
| | | VDCSEAGQGDVSIGIKCAPGVVGPVEADIDFDIIKNDNDTFTVKYTPPGAGHYTIMVL |
| | | FANQEIPASPFHIKVDPSHDASKVKAEGPGLSRTGVEVGKPTHFTVLTKGAGKAKLD |
| | | VHFAGAAKGEAVRDFEIIDNHDYSYTVKYTAVQQGNMAVTVTYGGDPVPKSPFVV |
| | | NVAPPLDLSKVKVQGLNSKVAVGQEQAFSVNTRGAGGQGQLDVRMTSPSRRPIPCK |
| | | LEPGGGAEAQAVRYMPPEEGPYKVDITYDGHPVPGSPFAVEGVLPPDPSKVCAYGPG |
| | | LKGGLVGTPAPFSIDTKGAGTGGLGLTVEGPCEAKIECQDNGDGSCAVSYLPTEPGE |
| | | YTINILFAEAHIPGSPFKATIQPVFDPSKVRASGPGLERGKAGEAATFTVDCSEAGEAE |
| | | LTIEILSDAGVKAEVLIQNNADGTYHITYSPAFPGTYTITIKYGGHPIPKFPTRVHVQPA |
| | | VDTSGIKVSGPGVEPHGVLREVTTEFTVDARSLTATGGNHVTARVLNPSGAKTDTYV |
| | | TDNGDGTYRVQYTAYEEGVHLVEVLYDEVAVPKSPFRVGVTEGCDPTRVRAFGPGL |
| | | EGGLVNKANRFTVETRGAGTGGLGLAIEGPSEAKMSCKDNKDGSCTVEYIPFTPGDY |
| | | DVNITFGGQPIPGSPFRVPVKDVVDPGKVKCSGPGLGTGVRARVPQTFTVDCSQAGR |
| | | APLQVAVLGPTGVAEPVEVRDNGDGTHTVHYTPATDGPYTVAVKYADQEVPRSPFK |
| | | IKVLPSHDASKVRASGPGLNASGIPASLPVEFTIDARDAGQGLLTVQILDPEGKPKKA |
| | | NIRDNGDGTYTVSYLPDMSGRYTITIKYGGDEIPYSPFRIHALPTGDASKCLVTVSIGG |
| | | HGLGACLGPRIQIGEETVITVDAKAAGKGKVTCTVSTPDGAELDVDVVENHDGTFDI |
| | | YYTAPEPGKYVITIRFGGEHIPNSPFHVLACDPLPHVEEPAEMLQMRQPYAPLRPGTC |
| | | PTHWATEEPVVPVEPLESMLRPFNLVIPFTVQKGELTGEVRMPSGKTARPNITDNKD |
| | | GTITVRYAPTEKGLHQMGIKYDGNHIPGSPLQFYVDAINSRHVSAYGPGLSHGMVNK |
| | | PATFTIVTKDAGEGGLSLAVEGPSKAEITCKDNKDGTCTVSYLPTAPGDYSIIVRFDD |
| | | KHIPGSPFTAKITGDDSMRTSQLNVGTSTDVSLKITEGDLSQLTASIRAPSGNEEPCLL |
| | | KRLPNRHIGISFTPKEVGEHVVSVRKSGKHVTNSPFKILVGPSEIGDASKVRVWGKGL |
| | | SEGQTFQVAEFIVDTRNAGYGGLGLSIEGPSKVDINCEDMEDGTCKVTYCPTEPGTYI |
| | | INIKFADKHVPGSPFTVKVTGEGRMKESITRRRQAPSIATIGSTCDLNLKIPGNWFQM |
| | | VSAQERLTRTFTRSSHTYTRTERTEISKTRGGETKREVRVEESTQVGGDPFPAVFGDF |
| | | LGRERLGSFGSITRQQEGEASSQDMTAQVTSPSGKTEAAEIVEGEDSAYSVRFVPQE |
| | | MGPHTVTVKYRGQHVPGSPFQFTVGPLGEGGAHKVRAGGTGLERGVAGVPAEFSIW |
| | | TREAGAGGLSIAVEGPSKAEIAFEDRKDGSCGVSYVVQEPGDYEVSIKFNDEHIPDSP |
| | | FVVPVASLSDDARRLTVTSLQETGLKVNQPASFAVQLNGARGVIDARVHTPSGAVEE |
| | | CYVSELDSDKHTIRFIPHENGVHSIDVKFNGAHIPGSPFKIRVGEQSQAGDPGLVSAY |
| | | GPGLEGGTTGVSSEFIVNTQNAGSGALSVTIDGPSKVQLDCRECPEGHVVTYTPMAP |
| | | GNYLIAIKYGGPQHIVGSPFKAKVTGPRLSGGHSLHETSTVLVETVTKSSSSRGASYSS |
| | | IPKFSSDASKVVTRGPGLSQAFVGQKNSFTVDCSKAGTNMMMVGVHGPKTPCEEVY |
| | | VKHMGNRVYNVTYTVKEKGDYILIVKWGDESVPGSPFKVNVP |
| 37 | Mouse ACON | MAPYSLLVTRLQKALGVRQYHVASVLCQRAKVAMSHFEPSEYIRYDLLEKNINIVRK |
| | (aconitate | RLNRPLTLSEKIVYGHLDDPANQEIERGKTYLRLRPDRVAMQDATAQMAMLQFISSG |
| | hydratase) | LPKVAVPSTIHCDHLIEAQVGGEKDLRRAKDINQEVYNFLATAGAKYGVGFWRPGS |
| | | GIIHQIILENYAYPGVLLIGTDSHTPNGGGLGGICIGVGGADAVDVMAGIPWELKCPK |
| | | VIGVKLTGSLSGWTSPKDVILKVAGILTVKGGTGAIVEYHGPGVDSISCTGMATICNM |
| | | GAEIGATTSVFPYNHRMKKYLSKTGRTDIANLAEEFKDHLVPDPGCQYDQVIEINLN |
| | | ELKPHINGPFTPDLAHPVADVGTVAEKEGWPLDIRVGLIGSCTNSSYEDMGRSAAVA |
| | | KQALAHGLKCKSQFTITPGSEQIRATIERDGYAQILRDVGGIVLANACGPCIGQWDRK |
| | | DIKKGEKNTIVTSYNRNFTGRNDANPETHAFVTSPEIVTALAIAGTLKFNPETDFLTG |
| | | KDGKKFKLEAPDADELPRSDFDPGQDTYQHPPKDSSGQRVDVSPTSQRLQLLEPFDK |
| | | WDGKDLEDLQILIKVKGKCTTDHISAAGPWLKFRGHLDNISNNLLIGAINIENGKANS |
| | | VRNAVTQEFGPVPDTARYYKKHGIRWVVIGDENYGEGSSREHAALEPRHLGGRAIIT |
| | | KSFARIHETNLKKQGLLPLTFADPSDYNKIHPVDKLTIQGLKDFAPGKPLKCVIKHPN |
| | | GTQETILLNHTFNETQIEWFRAGSALNRMKELQQ |
| 38 | Mouse PRVA | MSMTDVLSAEDIKKAIGAFAAADSFDHKKFFQMVGLKKKNPDEVKKVFHILDKDKS |
| | (parvalbumin | GFIEEDELGSILKGFSSDARDLSAKETKTLLAAGDKDGDGKIGVEEFSTLVAES |
| | alpha) | |
| 39 | Mouse HBA | MVLSGEDKSNIKAAWGKIGGHGAEYGAEALERMFASFPTTKTYFPHFDVSHGSAQV |
| | (hemoglobin | KGHGKKVADALASAAGHLDDLPGALSALSDLHAHKLRVDPVNFKLLSHCLLVTLAS |
| | subunit alpha) | HHPADFTPAVHASLDKFLASVSTVLTSKYR |
| 40 | Mouse HBAZ | MSLMKNERAIIMSMWEKMAAQAEPIGTETLERLFCSYPQTKTYFPHFDLHHGSQQLR |
| | (hemoglobin | AHGFKIMTAVGDAVKSIDNLSSALTKLSELHAYILRVDPVNFKLLSHCLLVTMAARF |
| | subunit zeta) | PADFTPEVHEAWDKFMSILSSILTEKYR |
| 41 | Mouse HSP7C | MSKGPAVGIDLGTTYSCVGVFQHGKVEIIANDQGNRTTPSYVAFTDTERLIGDAAKN |
| | (heat shock | QVAMNPTNTVFDAKRLIGRRFDDAVVQSDMKHWPFMVVNDAGRPKVQVEYKGET |
| | cognate 71) | KSFYPEEVSSMVLTKMKEIAEAYLGKTVTNAVVTVPAYFNDSQRQATKDAGTIAGL |
| | | NVLRIINEPTAAAIAYGLDKKVGAERNVLIFDLGGGTFDVSILTIEDGIFEVKSTAGDT |
| | | HLGGEDFDNRMVNHFIAEFKRKHKKDISENKRAVRRLRTACERAKRTLSSSTQASIEI |
| | | DSLYEGIDFYTSITRARFEELNADLFRGTLDPVEKALRDAKLDKSQIHDIVLVGGSTRI |
| | | PKIQKLLQDFFNGKELNKSINPDEAVAYGAAVQAAILSGDKSENVQDLLLLDVTPLS |
| | | LGIETAGGVMTVLIKRNTTIPTKQTQTFTTYSDNQPGVLIQVYEGERAMTKDNNLLG |
| | | KFELTGIPPAPRGVPQIEVTFDIDANGILNVSAVDKSTGKENKITITNDKGRLSKEDIER |
| | | MVQEAEKYKAEDEKQRDKVSSKNSLESYAFNMKATVEDEKLQGKINDEDKQKILD |
| | | KCNEIISWLDKNQTAEKEEFEHQQKELEKVCNPIITKLYQSAGGMPGGMPGGFPGGG |
| | | APPSGGASSGPTIEEVD |
| 42 | Mouse TNNI2 | MGDEEKRNRAITARRQHLKSVMLQIAATELEKEESRRESEKENYLSEHCPPLHIPGSM |
| | (troponin I) | SEVQELCKQLHAKIDVAEEEKYDMEVKVQKSSKELEDMNQKLFDLRGKFKRPPLRR |
| | | VRMSADAMLKALLGSKHKVCMDLRANLKQVKKEDTEKERDLRDVGDWRKNIEEK |
| | | SGMEGRKKMFESES |
| 43 | Mouse CASQ1 | MRATDRMGARAVSELRLALLFVLVLGTPRLGVQGEDGLDFPEYDGVDRVINVNAK |
| | (calsequestrin-1) | NYKNVFKKYEVLALLYHEPPEDDKASQRQFEMEELILELAAQVLEDKGVGFGLVDS |
| | | EKDAAVAKKLGLTEEDSVYVFKGDEVIEYDGEFSADTLVEFLLDVLEDPVELIEGER |
| | | ELQAFENIEDEIKLIGYFKSKDSEHYKAYEDAAEEFHPYIPFFATFDSKVAKKLTLKLN |
| | | EIDFYEAFMEEPMTIPDKPNSEEEIVSFVEEHRRSTLRKLKPESMYETWEDDLDGIHIV |
| | | AFAEEADPDGYEFLETLKAVAQDNTENPDLSIIWIDPDDFPLLVPYWEKTFDIDLSAP |
| | | QIGVVNVTDADSIWMEMDNEEDLPSADELEDWLEDVLEGEINTEDDDDDDDDDDD |
| | | DDDDDD |
| 44 | Mouse OBSCN | MDHSFSGAPRFLTRPKAFVVSVGKDATLSCQIVGNPTPHVSWEKDRQPVEAGARFRL |
| | (obscurin) | AQDGDVYRLTILDLALGDSGQYVCRARNAIGEAFAAVGLRVDSEGTCAEQAPHFLL |
| | | RPTSIRVREGADATFRCRVGGSPQPAVSWSKDGRRLGPPDAPHVRVEEHGESSALRI |
| | | RSARPRDGGTYEVRAENPLGSASAAAALVVDSDAEVAGPPGTSTATLLAHLQQRRE |
| | | AMRAEGIPPSPPGAGTRTCTVTEGKHARLSCFVTGEPKPETVWKKDGQLVTEGRRH |
| | | VVYEDEQENFVLKILFCKQSDRGLYTCTASNLVGQTYSSVLVVVREPTVPFKKRLQD |
| | | LEVREKESATFQCEVAQPATEAAWFKEETRLWASAKYDIEEEGTERRLTVRNVSAD |
| | | DDAVYICETTEGSRTVAELSVQGNLTRKLPRKTAVRTGDTAIFWVELAVPEGPVRWL |
| | | RNQEEMVAGGRVAITAEGTCHTLTIFQCTLEDMGEVAFVSGGCRTTTQFCVSAPRRP |
| | | PLYPPADPVVKAKTESSVTLSWSAPPHGDRPVTIDGYVLEKRKLGAYAWSRCHEAG |
| | | WLATTEFTITGVAEEGDFQFRVSAINHFGQGPYLEFPGTMHLVPMLAVKTPLKAVEA |
| | | VEGGEVTFSVDLTVASSGEWELDGEALKASSIYVIRCDRTRHMLTIREVPARLHGAQ |
| | | LKFVANGIETSIQMVVRAALGLPSSKLPAAAAREVLAQLHEEAQLLAELSDQAAAVT |
| | | WLKDGRELSLGPKYEMQVSAGRRALLVRDVAQDDAGLYECVSRGSRTAYQLLVQD |
| | | ITDGYRDWGPAGPQKHMCKCAGAKIARYLGSSCYRFLQYDKGVWHWLEAALDTR |
| | | QGKGTSSCSLHEKPKLVFAKGQQAHSEVKAEAGNSATLSCEVTQAQTEVTWFKDGK |
| | | KLSSSSKVRMEASGCSRRLVVQQAGKADAGEYSCEAGGQKLSFRLDVAEPKLVFAK |
| | | GQQAHSEVKAEAGASATLSCEVAQAQTEVTWFKDGKKLSSSSKVRMEASGCSRRL |
| | | VVQQAGKADAGEYSCEAGGQKLSFRLDVAEPKMVFAKEQQARSEVKAEAGASATL |
| | | SCEVAQAQTEVTWFKDGKKLSSSSKVRMEASGCSRRLVVQQAGKADAGEYSCEAG |
| | | GQKLSFRLDVTEPKLVFAKEQQARSEVKAEVGNSATLSCEVAQAQTEVTWFKDGKK |
| | | LSSSSKVRMEASGCSRRLVVQQAGKADAGEYSCEAGGQKLSFRLDVAEPKLVFAKE |
| | | QQARSEVKAEAGNSATLSCEVAQAQTEVTWFKDGKKLSSSSKVRMEASGCSRRLVV |
| | | QQAGKADAGEYSCEAGGQKLSFHLDVTEPKLVFAKEQQAHSEVKAEAGASATLSCE |
| | | VAQAQTEVTWFKDGKKLSSSSKVRMEASGCSRRLVVQQAGKADAGEYSCEAEGQK |
| | | LSFRLDVAEPKLVFAKEQQARSEVKAEAGASATLSCEVAQAQTEVTWFKDGKKLSS |
| | | SSKVRVEASGCSRRLVVQQAGKADAGEYSCEAGGQKLSFRLDVAEPKLVFAKEQQA |
| | | NSEVKAEAGASATLSCEVAQAQTEVTWFKDGKKLSSSSKVRVEASGCSRRLVVQQA |
| | | GKADAGEYSCEAGGQKLSFRLDVAEPKLAFAKEQQAHSEVKAEAGASATLSCEVAQ |
| | | AQTEVTWFKDGKKLSSSSKVRVEASGCSRRLVVQQAGKADAGEYSCEAGGQKLSF |
| | | RLDVAEPKLAFAKEQQAHSEVKAEAGASATLSCEVAQAQTEVTWFKDGKKLSSSSK |
| | | VRVEASGCSRRLVVQQAGKADAGEYSCEAGGQKLSFRLDVAEPKLAFAKEQQAHS |
| | | EVKAEAGASATLSCEVAQAQTEVTWFKDGKKLSSSSKVRVEASGCSRRLVVQQAG |
| | | KADAGEYSCEAGGQKLFFRLDVAEPKLMFAKEQQAHSEVKAEAGASATLSCEVAQ |
| | | AQTEVTWFKDGKKLSSSSKVRVEASGCSRRLVVQQAGKADAGEYSCEAGGQKLSF |
| | | RLDVAEPKLVFAKEQQAHSEVKAEAGASATLSCEVAQAQTEVTWFKDGKKLSSSSK |
| | | VRVEASGCSRRLVVQQVGKADAGEYSCEARGQKLSFRLDVADTRLMFAKEQQART |
| | | EVKAEAGNSATLSCEVAQAQTEVTWFKDGKKLSSSSKVRVEASGCSRRLVVQQAG |
| | | KADAGEYSCEAGGQKLSFRLDVAEAESQIPERPSRREPLVVKEHETIILTATIAAPSVA |
| | | AVTWLKDGVEIRRSKRHEATSLGDTHTLTVRGAQVLDSAIYSCRVGKEGQDFPVQV |
| | | EEVAAKFSKPLEPVEGELGGTVMLVCELSPEQAEVVWRCGNTQLRPGKRFQMTSEG |
| | | PRRTLTVSGLREDDAEEYVCESRDDRTSARLTVKVPRVVKFTSGLSAMVAEEGQEA |
| | | TFQCVVSPSDAGVTWYKDGMQLQPSEKFVMVESGASRSLTILGLTLEDAGQVTVEA |
| | | EGASSSAALRVREAPVLFKKKLEPQTVEERTSVTLEVELTRPWPEVKWTRNAAVLTP |
| | | SENVEIRAEGARHCLVLRSVGFADRGFFGCETPDDKTQAKLNVEMRQVRLVRGLQE |
| | | VEAKEQGTASMDVELSHAEVEGSWTRDGLRLQPGPKCHLAVQGPVHILTLSALQPQ |
| | | DSGLVAFRAEGVHTSARLIVTELPVSFTRVLQDVVATQKEKVTLECELSRPVDVRWL |
| | | KDGVELRAGKAIGIVAQGTCRSLVIYRCETGDQGVYVCDALDAQTSASLRVQGRTY |
| | | TLIFRRVLAEDAGEVKFVAENAESRAHLRVKELPVTLLRPLRDKIAMEKHRGVLECQ |
| | | VSRASAQVRWFKGGVELQSGPKYEVVSDGLYRKLVINDVQPEDEDTYTCDAGNVK |
| | | TSAQFFVEEQSITIVRGLKDMTVMEPAPAWFECETSIPSVRPPKWLLGKTVLQAGGN |
| | | VGLEQDGTVHRLTLHKTCSTMTGPVHFTIGKSRSSAQLVVSDIPVVLTRPLEPKAGRE |
| | | LQSVVLSCDFRPAPKAVQWYKDDTPLSPSEKFKMALEGQMAELRILRLTPADAGVY |
| | | RCQAGSAQSSAEVTVEAREVTVIQPLQDAEAMEEGRVCFSCELSHKDEDIEWSLNGT |
| | | PLYNDSFHEISHEGCLHTLVLKSVRQADTGTVCATSPKVSVSARLVVKGKPVVFLKA |
| | | LDDVSAEERGTLTLQCEVSDPEARVVWRKDGVELGPSDKYDFLHKAGARGLTVHD |
| | | LSHEDAGLYTCQVGSKETQSKVSVHDLHVGITKRLKTVEVLEGESCSFECVLSHESPS |
| | | DPAVWTVGGKTVGGSGHFHAVRQGRKYTLTVKDAALSDAGEVVFSVLGLTSKASLI |
| | | IRERPVDITKPLEDQRTTLGEDVMLSCELSRAGTSVRWLKDGKAIRKSQKYDLLSEG |
| | | TRAVLVVRKASLKDSGEYTCETEASKSTAKLCVEEKANRFTEELADLQVEEKGRAV |
| | | FTCKTEHPASVVTWRKGLLELRASGKHVPSQEGLTLKLTINALERTDSDTYTCDIGQ |
| | | ARTQARLLVHGQKVRVIEDLEDTAVQEGSSAKFCCRIAPADYGPVHWFLDKTPLHS |
| | | NELNEITVQPGGYHVLTLRQLALKDSGTVYFEAGDQRTSAALRVTEKPSIFSRPLTDV |
| | | TVTEGEDLTLVCETTTVDSSVRWTKDGKTLRPSARCQLSHEGCQAQLLITATTPQDG |
| | | GRYKCEIGGASSSSIVRVHALPVRFRESLKDVEVPEGKAATLRCVLSSVAAPVEWRH |
| | | GDDVLKSSNKYSLRQEGAVLELVIRDLQPQDSGQYSCSFGDQTTSATLTVKTSSAQF |
| | | VGKLRNKEATEGTTVTLRCELTKEAPVEWKKGTETLRNGDKYSLKQDGAVCELQIC |
| | | SLLVADAGEYSCVCGQEKTSATLTVKALLVHFVRRLRSKEATEGDTTTLQCELSKAA |
| | | PVEWRKGTETLRDGDRYSLKQDGAVCELQIRSLTIADAGEYLCTCGQEKTSATLTVR |
| | | ALPAKFKDSLKNEEATEGTTATLSCELSKAAPVTWKKGPKTLQSGDKYVLRQDGAV |
| | | CGLQIHGLTMADAGEYSCVCGQEKTSATLTVRGLPAKFIEDLRSQEATEGATAILRC |
| | | ELSKAAPVEWRKGSETLKDGDRYTLRQDGAVCELQIRGLAVVDTGTYSSLPTKFTE |
| | | GLRNEEATEGTMATLRCQMSKAAPVEWRKGSETLRDGDRYSLRQDGAMCELQIRG |
| | | LTIEDSGEYTCVCGQEKTSATLSVKALPSRFIEDLRSQEATEGTMATLRCQMSKTAPV |
| | | EWKKGSETLRDGGRYSLRQDGPVCELQICDLAVEDAGEYSCVCGQEKTSATLSIKAL |
| | | PPRFIEDLRSQEATEGTMATLRCQMSKAAPVEWRKGSETLGDGGRYSLRQNGAVCE |
| | | LQIHDLAVEDTGEYSCVCGQEKTSATLNVKALPPRFIEDLRSQEATEGTMATLRCQM |
| | | SKAAPVEWRKGSETLRDGGRYSLRQDGAVCELQIHDLDVEDAGQYSCVCGQEKTS |
| | | AVLTVDALPPKFTEGLKKEEATEGTMVTLRCQMSKEATVEWRKGAKTLSDGGRYS |
| | | LRQDGAMCELQICGLAVEDAGEYSCVCGQEKTSATLSVKALPPRFIEDLRSQEATEG |
| | | TMATLRCQMSKAAPVEWRKGSETLRDGDRYSLRQDGAVCELQIRDLAVEDAGEYL |
| | | CVCGQEKTSATLSVKALPPRFIEDLRSQEATEGTMATLRCQMSKAAPVEWRKGSKT |
| | | LRDGDRYSLRQDGAMCELQICDLAVEDTGDYSCVCGQEKTSATLSVKALPPRFIEDL |
| | | RSQEAREGTVATLRCRMSKAAPVEWRKGSETLKDGDRYSLRQEGNLCELQIRDLAV |
| | | EDTEEYSCVCGQEKTSATLSVKALPAKFIEDLRSQEAPESSTVTLRCKLSKKASVVW |
| | | KKGSETLRNGARYSLRQDGAVCELEIRDLTVEDTGEYSCTCGQERTSATLSIMAPQV |
| | | VFQQPLQNLQAEEGSMASLRCELSVPNAAMVWSKGGLELQGDTRREARQQGCVAE |
| | | LLLRDLRREDAGEYSCTCGSQTTSATLMVTAAPVRFLRELQAQDVDEGATARLRCE |
| | | LSREAVSVEWRKGSLQLFPCAKYQMVQEGTTAELLVHGVEQEDAGEYTCDAGHTQ |
| | | SIARLSVRAPKPKFKTDLQSTEQEAGGTARLCCQLSEAEPGTPVQWLKEGVELHVGS |
| | | KYEMRRQGAVCELLIHGLEAKDTGEYACLVGGQKTLASLRVKEPEVTIVRGLVDME |
| | | VQADEDVEFTCKVSQAGATDVQWHLQGLPLQSNEVTEVAVLADGCTHVLQLKGVT |
| | | LEDAGTVSFHVGGLSSSAQLTVRVPEVTVLEPLKDVQLSEGQDAHFRCRLSRASGQE |
| | | ARWALGGVPLQCNEMNDITVEQGTLYLLTLHKVTLEDAGTITLQVGSCSSEAQLKV |
| | | TAKNTVLRGLENVDALEGGEALFECQLSQPEVAAHTWLLDDEPVHTSEKVEVVYFE |
| | | NGLRHLLLLKNLKPQDSCRVTFLAGDVVTSAFLTVRGWRLEVLEPPHDASVKAGMQ |
| | | VRFTCILSEAVPVGEATWYINGAAIQPDDTDWTVTTDGSHHALTLSNAQPQHAGEVT |
| | | FAARDAVASARLSVLALPDPPEDAEVVGRSDHSVTLSWVAPMSDGGGGLCGYRVE |
| | | MKEASTGQWQLCHDLVPGPECVVDDLVPGKTYRFRVAAVGPAGAGEPVHLPQMV |
| | | KIAPAPAPAPAPAPAPAPETRQAVVGEDICLELEVAADGGEVVWHKGTERIQPGGHF |
| | | EVLSRGQRQMLVIKGFRTEDQGEYRCGPIQGLPSSGAATFNVVMTSGSGDEVPAQPS |
| | | LPPEAAQEGDLHLLWEALARKRRMSREPTLDSISELPEEDSRVQHLRQEAEETAPDLS |
| | | EGYSTADELARTGEADLSHTSSDDESRAGTPSLVTYLKKAGGPGISPLASKHEAQVT |
| | | TSVKPQKQQEPVVPTCPPPGDLSAADLMDPSLDKAAVKIQAAFKGYKVRKEMKQQE |
| | | GPVFSRTFGDTEAQVGDVLRLECVLATKTDMRACWLKDGIELTDGRHYHIDQLKDG |
| | | TCSLLVTGLAPTDSGRYTCQVSTKSGRVSHSACVVVSGTESEAESSSGGELDDAFRR |
| | | AARRLHRLFRTKSPAELSEEELFLSADEGPGEPEEPADWQTYREDENFVCIRFESLAE |
| | | ARQAVTCFRNMFATMGIGVEISLGEQGPRGVEMRIGKVAPTVTPAVPLAKTPGLQTS |
| | | DAAPVFLTELQNQDVQDGYPMSFDCVVTGQPVPSVRWFKDGKLLEEDDHYMINED |
| | | QQGGHQLIITAVVPADMGVYRCLAENSMGVSSTKAELRVELTSTDYDTAADATETS |
| | | SYFSAQGYLSSREQEGTESDEGQLPQVLEELKDLQVAPGTRLAKFQLKVKGYPAPKL |
| | | YWFKDGQPLTTSDHIRMTDKKTLHTLEIVSVTREDSGQYAAYISNAVGAAYSSARLL |
| | | VRGPSEPEEKPASDVHERLVPPRILEKFTPKKVKRGSSITFSVKVEGHPAPSVHWLKE |
| | | EAEKGVLWIGPDTPGYTMASSSKQHSLVLLDVGRQHQGTYTCIATNPAGQALCSAS |
| | | LHISGLAKEEEQERVKEALISSFLQGTSQAVSAQMSESAGFADLVGQSKGESLVAEEA |
| | | HSHLSLAEVGTEEFLQKLTSQITEMVSAKISQAKLQVPGGDSDEETKTPSASPRHGRS |
| | | RPSSSVQESSSESEDGDSRGEIFDIYVVTADYLPLGAEQDAIILREGQYVEVLDSAHPL |
| | | RWLVRTKPTKSSPSRQGWVSPAYLDKRLKLSPEWGPTEAPEFPGEAVSEDEYRTRLS |
| | | SVIQELLSSEQAFVGELQFLESHHMKHLERSPRVPAAVASQKTVIFRNVQDISHFHSSF |
| | | LKELQGCGTDDDVAMCFIKNQEAFEKYLEFLVGRVQAESVVVSTPVQEFYKKYAEE |
| | | TLSAKDPTQPPPPPLQHYLEQPVERVQKYQALLKELIRNKARNRQNCALLEQAYAVV |
| | | SALPQRAENKLHVSLMENYPGTLEALGEPIRQGHFIVWEGAPGARMPWKGHNRHVF |
| | | LFRNYLVICKPRRDSRTDTFSYVFRNMMKLSSIDLNDQVEGDDRAFEVWHEREDSV |
| | | RKYLLQARTVIIKNSWVKEICGIQQRLAQPVWRPPEFEEELADCTAELGETVKLACR |
| | | VTGTPKPIVSWYKDGKPVEVDPHHILIEDPDGSCTLILDNLTGIDSGQYMCFAASAAG |
| | | NASTLGKILVQVPPRFVNKVRATPFVEGEDAQITCTVEGAPYPQIRWYKDGTLLAPG |
| | | NRYRMLNEPRSGVLVLVIQAASKEDLGHYECELVNRLGSTRGGGELYMQSPALRAR |
| | | DQHHREQIVAAVEDTSVEGSAHSAQDGADQQAASVLWRLLGSEALGPSPGDLPNTR |
| | | QSEPPAFEEAASQIPGAASGTPEVSQPGTHKGLEQETTSSGSQGWTVPIRVEGTAWPG |
| | | AGTGQLLLDVHSQVIMETTQRTYVCQAPDTGVTRAPSMQVTIEDVQVQVGDMAQF |
| | | DAVIEGHPPPIVTWYKGSTQLTSSARLSQRQDGTTYSLVLTDVAPHDAGVYTCVANN |
| | | AGGQVLCKAELLVHGGDKLDAENQVYRRKLHSFYDVQEEIGRGVFGFVKRVQHKG |
| | | NKMFCAAKFIPLRSKTRAQAYQERDILATLGHPLVTGLLDQFETRKTLILILELCSSEE |
| | | LLDRLFKKGVVTEAEVKVYIQQLVEGLHYLHSHGILHLDIKPPNILMVHPAREDIKIC |
| | | DFGFAQKITPSEPQYSKYGSPEFVSPEIIEQNPVSEGSDIWAMGVISYLSLTCSSPFAGE |
| | | SDRATLLNVLEGRVSWSSPTAAHLSEDAKDFIKATLQRTPRARPSTSQCLAHPWFLK |
| | | SMPAEEAHFINTKQLKFLLARSRWQRSLMSYKSILVMRSIPELLQGPPDSPSLGVARH |
| | | LRGEASGASSSSSSSDNELAPFARAKSLPPSPVTHSPLLHPRGFLRPSASLPEETEASMP |
| | | TADAAVPASPQSAGPPASPGCVPRHSVISSLFYQQAGEGAERGNKTSGAKRHPARRR |
| | | HLLKGGYIARALPGLREPLMEYSLLEEEAAREEQASLMTKTPSFETALRLPSSSVREV |
| | | PGRSHSLDNPPVTTGPSPEACKEQLLFPPSTGLTHETTAKDRGHKEGFLQESVPFPPM |
| | | SGDSRPGKQEGSSQDSCRGKPASSCHSELGSGSQEGCGPPSSQSLGSLPPQSLKKELST |
| | | SCGPLFSEQPQAAPFPTQVSPLLGSEKEPQDGSLSEGPVPVPSSSPGSASQVDASLDTE |
| | | GLSEAGDTCDFTPPPQRPQEQATTRKFSLESRGGYAGVAGYGTFAFGGDAGGMLGQ |
| | | GPLWARMAWAVSQSSEEQDEAATESPQPLESLGPIAEASGVPLRTSPSLTPWEEVEQ |
| | | VSLVQIRDLSGDAEAADTISLDISEVDPAYLNLSDLYDIKYLPFEFMIFRRVPKPIEQPE |
| | | SPGSETEAGQGLADFLEEAAWPWPGELGLRAGLEITEEPEEPGDLEALLGEAAVGRK |
| | | RKWSPSRGLFQFPGRCLSGEEPVELGLRQRVKASMAHISRILKGRPEGPEREGPPRKK |
| | | AGLASFRLSGLKGRDQELSDEAVVLGQSVTLACQVLAQPTAQATWSKDGVLLESSG |
| | | HLLISSTLKNFQLLTILVVKEEDLGTYTCCVSNPLGTAVTTGVLRKAERPSSSPRPEVG |
| | | ELYKDAVLLVWKPVESCGPVTYIVQCCIEGGSWTTLASDISDCCYLTGKLSRGGMYI |
| | | FRTACVSKAGMGPYSSPSEQVLLGGPNHLASEEESSRGRPAQLLPSTKTFAFQMQIRR |
| | | GRFSVVRQCREKASGRALAAKIVPYQPEDKTAVLREYEALKRLHHPHLAQLHAAYL |
| | | SPRHLVLILELCSGPELLPSLAERESYSESDVKDYLWQMLSATQYLHAQHILHLDLRS |
| | | ENMMVTEYNLLKVIDLGNAQSLDQEKVPAPENFKDYLETMAPELLEGQGAVPQTDI |
| | | WAIGVTAFIMLSGEYPESSEGTRDLQKGLRKGLIRLSRCYAGLSGGAVAFLQSSLCA |
| | | QPWGRPCASTCLQCGWLTEEGPTGSRPTPVTFPTVRLRAFVREREKRRALLYKKHNL |
| | | AQVR |
| 45 | Mouse F8VPN4 | MEHSKQIRILLLNEMEKLEKTLFRLEQGFELQFRLGPTLQGKAVTVYTNYPLPGEAFN |
| | (glycogen | REKFRSLDWENPTEREDDSDKYCKLHLQQSGSFQYYFLQGNEKSGGGYIVVDPILRI |
| | debranching | GVDNHVLPLDCVTLQTFLAKCLGPFDEWESRLRVAKESGYNMIHFTPLQTLGLSRSC |
| | enzyme) | YSLADQLELNPDFSRPSKRYTWSDVGQLVEKLKREWNILCITDVVYNHTATNSKWIL |
| | | EHPESAYNLVNSPHLKPAWVLDRALWHFSCDVADGKYREKGVPALIENDQHMNCI |
| | | RKIIWEDIFPRIQLWEFFQVDVHKAVEQFRRLLSQENRRVTKSEPKEHLKIIQDPEYRR |
| | | RGCAVDMDTALATFIPHDNGPAAIEECCNWFRKRLEELNSEKHHLTSCHQEQAVNC |
| | | LLGNVFYERLAGHGPKLGPVTRKYPLVTRYFTFPFGEMALSAEEALIHLPDKACFLM |
| | | AHNGWVMGDDPLRNFAEPGSDVYLRRELICWGDSVKLRYGNKPEDCPYLWAHMK |
| | | KYTEITATHFQGVRLDNCHSTPLHVAEYMLDAARKLQPNLYVVAELFTGSEELDNIF |
| | | VTRLGISSLIREAMSAYNSHEEGRLVYRYGGEPVGSFVQPCLRPLMPAIAHALFMDIT |
| | | HDNECPIVHRSAYDALPSTTVVSMACCASGSTRGYDELVPHQISVVAEERFYTKWNP |
| | | GASPADTGDVNVHSGIIAARCAINRLHQELGAKGFIQVYVDQVDEDIVAVTRHSPSIH |
| | | QSVVAVSRTAFRNPKTSFYSKEVPQMCIPGKIEEVVLEARTIERNTKPYKKDENSING |
| | | MPNMTVELREHIQLHESKIVRQAGVATKGPNEYIQEIEFENLSPGSVIIFRVSLDPHAQ |
| | | VAVGILRNHLTQFSSHFKSGSLAVDNADPILKIPFASIASKLTLAELNQVLYRCESEEQ |
| | | EDGGGCYDIPNWSSLKYAGLQGLMSVLAEIRPKNDLGHPFCENLRSGDWMIDYVSG |
| | | RLISRSGSIAEVGKWLQAMFFYLKQIPRYLIPCYFDAILIGAYTTLLDVAWKQMSSFV |
| | | QNGSTFVKHLSLGSVQMCGVGKCPCLPLLSPSLLDVPCRLNEITKEKEQCCASLAAG |
| | | LPHFSSGLFRCWGRDTFIALRGMLLVTGRYLEARNIILAFASTLRHGLIPNLLGEGTY |
| | | ARYNCRDAVWWWLQCIQDYCRTVPNGLDILKCPVSRMYPTDDSAPLPAGTLDQPLF |
| | | EVIQEAMQRHMQGIQFRERNAGPQIDRNMKDEGFNITAGIDEETGFVYGGNRFNCGT |
| | | WMDKMGESDRARNRGIPATPRDGSAVEIVGLCKSAVRWLLELSKKNIFPYHEVRVK |
| | | RHGKVVAVSYDEWNRKIQNNFEKLFHVSEDPSDPNEKHPNLVHKRGIYKDSYGASS |
| | | PWCDYQLRPNFTIAMVVAPELFTAEKAWKALEIAEKKLLGPLGMKTLDPDDMVYC |
| | | GVYDNALDNDNYNLARGFNYHQGPEWLWPIGYFLRAKLYFSKMMGPETAAKTVFL |
| | | VKNVLSRHYVHLERSPWKGLPELTNENGQYCPFSCETQAWSMAVVLETLYDL |
| 46 | Mouse EF1A2 | MGKEKTHINIVVIGHVDSGKSTTTGHLIYKCGGIDKRTIEKFEKEAAEMGKGSFKYA |
| | (elongation | WVLDKLKAERERGITIDISLWKFETTKYYITIIDAPGHRDFIKNMITGTSQADCAVLIV |
| | factor 1-alpha 2) | AAGVGEFEAGISKNGQTREHALLAYTLGVKQLIVGVNKMDSTEPAYSEKRYDEIVK |
| | | EVSAYIKKIGYNPATVPFVPISGWHGDNMLEPSPNMPWFKGWKVERKEGNASGVSL |
| | | LEALDTILPPTRPTDKPLRLPLQDVYKIGGIGTVPVGRVETGILRPGMVVTFAPVNITT |
| | | EVKSVEMHHEALSEALPGDNVGFNVKNVSVKDIRRGNVCGDSKADPPQEAAQFTSQ |
| | | VIILNHPGQISAGYSPVIDCHTAHIACKFAELKEKIDRRSGKKLEDNPKSLKSGDAAIV |
| | | EMVPGKPMCVESFSQYPPLGRFAVRDMRQTVAVGVIKNVEKKSGGAGKVTKSAQK |
| | | AQKAGK |
| 47 | Mouse AATC | MAPPSVFAQVPQAPPVLVFKLTADFRDDPDPRKVNLGVGAYRTDESQPWVLPVVRK |
| | (aspartate | VEQKIANDNSLNHEYLPILGLAEFRSCASRLVLGDNSLAIRENRVGGVQSLGGTGAL |
| | aminotransferase | RIGADFLGRWYNGTDNKNTPIYVSSPTWENHNAVFSAAGFKDIRPYCYWDAEKRGL |
| | | DLQGFLNDLENAPEFSIFVLHACAHNPTGTDPTPEQWKQIAAVMQRRFLFPFFDSAY |
| | | QGFASGDLEKDAWAIRYFVSEGFELFCAQSFSKNFGLYNERVGNLTVVGKESDSVLR |
| | | VLSQMEKIVRITWSNPPAQGARIVAATLSDPELFKEWKGNVKTMADRILTMRSELRA |
| | | RLEALKTPGTWSHITEQIGMFSFTGLNPKQVEYLVNEKHIYLLPSGRINMCGLTTKNL |
| | | DYVATSIHEAVTKIQ |
| 48 | Mouse KAD1 | MEEKLKKAKIIFVVGGPGSGKGTQCEKIVQKYGYTHLSTGDLLRAEVSSGSERGKKL |
| | (adeny late | SAIMEKGELVPLDTVLDMLRDAMLAKVDSSNGFLIDGYPREVKQGEEFEQKIGQPTL |
| | kinase | LLYVDAGAETMTQRLLKRGETSGRVDDNEETIKKRLETYYNATEPVISFYDKRGIVR |
| | isoenzyme 1) | KVNAEGTVDTVFSEVCTYLDSLK |
| 49 | Mouse G6PI | MAALTRNPQFQKLLEWHRANSANLKLRELFEADPERFNNFSLNLNTNHGHILVDYS |
| | (glucose-6- | KNLVNKEVMQMLVELAKSRGVEAARDNMFSGSKINYTENRAVLHVALRNRSNTPI |
| | phosphate | KVDGKDVMPEVNRVLDKMKSFCQRVRSGDWKGYTGKSITDIINIGIGGSDLGPLMV |
| | isomerase) | TEALKPYSKGGPRVWFVSNIDGTHIAKTLASLSPETSLFIIASKTFTTQETITNAETAKE |
| | | WFLEAAKDPSAVAKHFVALSTNTAKVKEFGIDPQNMFEFWDWVGGRYSLWSAIGL |
| | | SIALHVGFDHFEQLLSGAHWMDQHFLKTPLEKNAPVLLALLGIWYINCYGCETHALL |
| | | PYDQYMHRFAAYFQQGDMESNGKYITKSGARVDHQTGPIVWGEPGTNGQHAFYQL |
| | | IHQGTKMIPCDFLIPVQTQHPIRKGLHHKILLANFLAQTEALMKGKLPEEARKELQAA |
| | | GKSPEDLEKLLPHKVFEGNRPTNSIVFTKLTPFILGALIAMYEHKIFVQGIMWDINSFD |
| | | QWGVELGKQLAKKIEPELEGSSAVTSHDSSTNGLISFIKQQRDTKLE |
| 50 | Mouse DESM | MSQAYSSSQRVSSYRRTFGGAPGFSLGSPLSSPVFPRAGFGTKGSSSSMTSRVYQVSR |
| | (desmin) | TSGGAGGLGSLRSSRLGTTRAPSYGAGELLDFSLADAVNQEFLATRTNEKVELQELN |
| | | DRFANYIEKVRFLEQQNAALAAEVNRLKGREPTRVAELYEEEMRELRRQVEVLTNQ |
| | | RARVDVERDNLIDDLQRLKAKLQEEIQLREEAENNLAAFRADVDAATLARIDLERRI |
| | | ESLNEEIAFLKKVHEEEIRELQAQLQEQQVQVEMDMSKPDLTAALRDIRAQYETIAA |
| | | KNISEAEEWYKSKVSDLTQAANKNNDALRQAKQEMMEYRHQIQSYTCEIDALKGT |
| | | NDSLMRQMRELEDRFASEANGYQDNIARLEEEIRHLKDEMARHLREYQDLLNVKM |
| | | ALDVEIATYRKLLEGEESRINLPIQTFSALNFRETSPEQRGSEVHTKKTVMIKTIETRD |
| | | GEVVSEATQQQHEVL |
| 51 | Mouse KCRS | MASAFSKLLTGRNASLLFTTLGTSALTTGYLLNRQKVSADAREQHKLFPPSADYPDL |
| | (creatine kinase | RKHNNCMAECLTPTIYAKLRNKMTPSGYTLDQCIQTGVDNPGHPFIKTVGMVAGDE |
| | S-type) | ESYEVFADLFDPVIKLRHNGYDPRVMKHPTDLDASKITHGQFDERYVLSSRVRTGRSI |
| | | RGLSLPPACSRAERREVENVAITALEGLKGDLAGRYYKLSEMTEQDQQRLIDDHFLF |
| | | DKPVSPLLTCAGMARDWPDARGIWHNYDKTFLIWINEEDHTRVISMEKGGNMKRVF |
| | | ERFCRGLKEVERLIQERGWEFMWNERLGYILTCPSNLGTGLRAGVHVRIPKLSKDPR |
| | | FSKILENLRLQKRGTGGVDTAAVADVYDISNIDRIGRSEVELVQIVIDGVNYLVDCEK |
| | | KLERGQDIKVPPPLPQFGRK |
| 52 | Mouse | MRKHRHLPLVAVFSLLLSGIATTHAQQHGDVKNGAAADIVFLVDSSWSAGKDRFLL |
| | E9PWQ3 | VQEFLSDVVESLAVGDNDFHFALVRLNGNPHTEFLLNTYHSKQEVLSHIVNMSYIGG |
| | (collagen, type | SNQTGKGLEYVIHSHLTEASGSRAADGVPQVIIVLTDGQSEDGFALPSAELKSADVN |
| | VI, alpha 3) | VFAVGVEGADERALGEVASEPLSMHVFNLENVTSLHGLVGNLVSCIHSSVNPERAG |
| | | DKESLKDITAQDSADIIFLIDGSQNTGNANFDVIRDFLVNVLERLSVGNQQVQVGVV |
| | | QYSEEPITMFSLNSYPSKAAVLDAVKGLSLVGGESANIGQALDFVVENHFTRAGGSR |
| | | VEEGVPQVLVLISAGPSSDEIRDSVVALKQASVFSFGLGAQAASRAELQHIATDDSLV |
| | | FTVPEFRSFGDLQEQILPYLVGVAQRHIVLQPPAIVTQVMEVNKRDIVFLVDGSSSLG |
| | | PSNFNAIRDFVTRVIQRLEIGQDLVQVSVAQYADTVKPEFYLNSYTNKRDAITAVRK |
| | | MRALNGSALYTGSSLDFVRNNLFTSSAGHRAAEGVPKLLVLITGGKSLDEVSQPAQE |
| | | LKRGSIMALAVGSKAADEDELKEIAFDSSLVFIPAEFRPAPLQNMLPSLMAPLRTLTG |
| | | TTEVHVNKRDIIFLLDGSDNVGKNNFPYVRDFVTNLVNSLDVGSDNIRVGLVQFSDT |
| | | PVTEFSLDTYQTKSELLAHLRRLQLKGGSGLNAGSALSYIHANHFTEAGGSRTREHV |
| | | PQLLLLLMAGPSEDAYLQAANALVRSGVLTFCVGTNRADKAELEHIAFNPSLVYLM |
| | | DDFRSLPSLPQQLIQPLTTYVSGGVQEVPLSQPESKRDILFLFDGSVNVLGQFPAVRDF |
| | | LYRIIEELDVKPDGTRVAIAQFSDDVRLESRFSEHQTKAEILNLVKKMKLKTGKALNL |
| | | GYALDYALRNIFVRSAGSRIEDNVQQFLVLLVAGRSSDAVAGPASSLKQRGVVPFIF |
| | | QAKNANPSELEQIVLSPAFILAAESLPKIGDLQSQIVSLLKAEQGSGPVSGEKDVVFLI |
| | | DGSEGVRSGFPLLKDFVQRVVESLDVGPDRVRVALVQYSDRTRPEFYLNSHMDQQG |
| | | VISAIRRLTLLGGPTPNTGAALEFVLRNILTSSTGSRIAEGVPQLLIVLTAEPSGDDVRG |
| | | PSVVLKQGGAVPIGIGIGNADISEMQTISFIPDFAVAIPTFRELGTIQQVISERVIQLNRE |
| | | ELSSLKPILTPSTGAGVGSKKDVVFLIDGSRNAGPEFQYIRTLIERIVEYLDIGFDTTRV |
| | | AVIQFSEDSKMEFPLNAHFSKDEVQNAVRRLRPKGGSQVYIGNALEYVLKNIFQRPL |
| | | GSRIEEGVPQFLVLISSGKSDDEVDDSAVELKQFGVAPLTIARHTDQEELVKISLSPEY |
| | | VYSVSTFRELPRLEQKLLTPITTLTSQQIHQILASTRYPPSVVESDAADIVFLIDSSDAV |
| | | KPDGIAHIRDFVSRIVRRLNIGPSKVRIGVVQFSNDVFPEFYLKTHKSQSSVLEAIRRLR |
| | | FKGGSPLNTGRALEFVARNLFVKSAGSRIEDGVPQHLVLFLGGKSQDDVARHAQVIS |
| | | SSGIVSLGIGDRNIDRTDLQTITNDPRLVFTVREFRELPNIEERVMLSFGPSGATPQPPG |
| | | VDLPSPSRPEKKKADIVFLLDGSINFRRDSFQEVLRFASEIVDTVYEDGDSIRVGLVQY |
| | | NSDPTDEFFLRDFSTKRQIIDAINKVVYKGGRHANTRVGIEHLLRNHFVPEAGSRLDE |
| | | RVPQIAFVITGGKSVEDAQDVSLALTQKGVKVFAVGVRNIDSEEVGKIASNSATAFR |
| | | VGSVQELSELSETVLETLHDAMHETLCPGVTDVSKACNLEVILGFDGSRDQNVFVSQ |
| | | KGLESKVDIILNRISQIQRISCSGNQLPTVRVSVMANTPSGPVEAFDFAEYQPELFEKF |
| | | RNMRSQRPYVLTADTLKLYQNKFRQSSPDTVKVVIHFTDGADGDMADLYRASEELR |
| | | QAGAQALILVGLERVANLERLMHLEFGRGFMYDRPLRLNLLDLDYELAEQLDNIAE |
| | | KACCGVPCKCSGERGDRGPIGSIGPKGISGEDGYRGYPGDEGGPGERGPPGVNGTQG |
| | | FQGCPGQRGVKGSRGFPGEKGELGEIGLDGLDGEEGDKGLPGSSGEKGSPGRRGDK |
| | | GPKGDKGERGDVGIRGDPGDSGRDSQQRGPKGETGDIGPMGLPGRDGIPGSPGDPGK |
| | | DGGSGRRGPAGAKGNRGGPGQPGFEGEQGTRGSQGPPGPIGPPGLIGEQGIPGPRGG |
| | | GGTAGAPGERGRTGPLGRKGEPGEPGPKGSIGNRGPRGETGDDGRDGVGSEGRRGK |
| | | KGERGFPGYPGPKGTPGEPGADGPPGPKGIRGRRGNSGPPGATGQKGDPGYPGPSGH |
| | | KGNRGDSVDQCALIQSIRDKCPCCYGPLECPVFPTELAFALDTSEGVTQDTFSRMREV |
| | | LLGIVGDLTIAESNCPRGARVAVVTYNNEVTTEIRFADSKKKSALLDSIQNLQVALTS |
| | | KQQSLETAMSFVARNTFKRVRSGFLMRKVAVFFSNKPTRASPQLREAVLKLSDAGIT |
| | | PLFLTSQEDRQLINALQINNTAVGHALVLPARRDLTDFLKNVLTCHVCLDICNIDPSC |
| | | GFGSWRPSFRDRRAAGSDVDIDLAFILDSSEATTLFQFNEMKKYIGYVIRQLDLSPDP |
| | | KASQHFARVAVVQQSTYESVDNASVPPVKVEFSLTDYGAKEKLLDFLSRRMTQLQG |
| | | TMGLGNAIEYTIENIFESAPNPRDLKIMVLMLTGDMQRQQLEEAQRAILQAKCKGYF |
| | | FVVLGIGRKVNIKEVYSFASEPNDVFFKFVDKSTELNEEPLMRFGRLLPSFVSSENAF |
| | | YLPPDLRKQCDWFQGDQPVKNGVKFGHKQINPPHTANSSLTSKVVTTMKPVTTTKP |
| | | TAIVNLPPAKPAPARPAPAQPVLAKPDPAKPAQARPAPAKPASAKLVPPQPVHVQPA |
| | | PAQTASVRPAPAKPAPPQPAAAKPVPAKPAVPAQPAPPQPAAAKPVPAKPAVPAQPA |
| | | PPQPAAAKPVPAKPAVPAQPAAAQPMPAQPVLTKSAAVKPASANKPVAAKPVATNT |
| | | ATATARPALAAKPAAAKPAATRPLAAAIRPVATKPEAPRQQAKPAATKPATTKPLAR |
| | | VSREVQVSEVTENSARLHWERPEPSSSFFYDLTVTSAHDQSLVLRQNLTVTDRVIGG |
| | | LLAGQLYHVVVVSYLQSQVRAIYQGSFNTKKTQPPPLQAAHRASSSTINLMVNTEPL |
| | | FLTKTDICKLSRDAGTCVDFKLLWHYDLESKSCKRFWYGGCGGNENRFHSQEECEK |
| | | MCSPDLLV |
| 53 | Mouse AMD1 | MPLFKLTGQGKQIDDAMRSFAEKVFASEVKDEGGRHEISPFDVDEICPISLHEMQAHI |
| | (AMP | FHMENLSMDGRRKRRFQGRKTVNLSIPQSETSSTKLSHIEEFISSSPTYESVPDFQRVQI |
| | deaminase 1) | TGDYASGVTVEDFEVVCKGLYRALCIREKYMQKSFQRFPKTPSKYLRNIDGEALVG |
| | | NESFYPVFTPPPKKGEDPFRTEDLPANLGYHLKMKAGVIYIYPDEAAANRDEPKPYP |
| | | YPNLDDFLDDMNFLLALIAQGPVKTYAHRRLKFLSSKFQVHQMLNEMDELKELKNN |
| | | PHRDFYNCRKVDTHIHAAACMNQKHLLRFIKKSYHIDADRVVYSTKEKSLTLKELFA |
| | | KLNMHPYDLTVDSLDVHAGRQTFQRFDKFNDKYNPVGASELRDLYLKTDNYINGE |
| | | YFATIIKEVGADLVEAKYQHAEPRLSIYGRSPDEWNKLSSWFVCNRIYCPNMTWMIQ |
| | | VPRIYDVFRSKNFLPHFGKMLENIFLPVFEATINPQAHPDLSVFLKHITGFDSVDDESK |
| | | HSGHMFSSKSPKPEEWTMENNPSYTYYAYYMYANITVLNSLRKERGMNTFLFRPHC |
| | | GEAGALTHLMTAFMIADNISHGLNLKKSPVLQYLFFLAQIPIAMSPLSNNSLFLEYAK |
| | | NPFLDFLQKGLMISLSTDDPMQFHFTKEPLMEEYAIAAQVFKLSTCDMCEVARNSVL |
| | | QCGISHEEKAKFLGNNYLEEGPVGNDIRRTNVAQIRMAYRYETWCYELNLIAEGLKA |
| | | TE |

An animal protein can comprise a rabbit protein (Table 2). A rabbit protein can comprise a rabbit muscle protein. A rabbit protein can comprise a rabbit blood protein. A rabbit protein can comprise a rabbit albumin protein. A rabbit protein can comprise a sequence that has at least or about 70%, at least or about 75%, at least or about 80%, at least or about 90%, at least or about 91%, at least or about 92%, at least or about 93%, at least or about 94%, at least or about 95%, at least or about 96%, at least or about 97% at least or about 98% at least or about 99%, or about 100% sequence identity to any one of SEQ ID NOs: 54-78. A rabbit albumin protein can comprise a sequence that has at least or about 70%, at least or about 75%, at least or about 80%, at least or about 90%, at least or about 91%, at least or about 92%, at least or about 93%, at least or about 94%, at least or about 95%, at least or about 96%, at least or about 97% at least or about 98% at least or about 99%, or about 100% sequence identity to SEQ ID NO: 54-78.

A food product may comprise a fragment or a truncation of a rabbit protein. The food product may include a fragment or truncation of any one of SEQ ID Nos: 54-78. A sequence of the fragment or truncation may be identical or substantially identical to a portion of any one of SEQ ID Nos: 54-78. A sequence of the fragment or truncation may be at least about 70%, 80%, 90%, 95%, 98%, or more identical to a portion of a sequence of any one of SEQ ID Nos: 54-78. A length of the fragment or truncation may be at least about 10%, 15%, 20%, 30%, 40%, 50%, 60%, 70%, 80%, 90%, or more of a length of any one of SEQ ID Nos: 54-78. A length of the fragment or truncation may be less than or equal to about 90%, 80%, 70%, 60%, 50%, 40%, 30%, 20%, 15%, 10%, or less than a length of any one of SEQ ID Nos: 54-78.

TABLE 2
Rabbit Proteins

| SEQ | | |
|---|---|---|
| ID NO | Description | Amino Acid Sequence |
| 54 | Rabbit KCRM | FGNTHNKYKLNYKSEEEYPDLSKHNNHMAKVLTPDLYKKLRDKETPSGFTL |
| | (Creatine | DDVIQTGVDNPGHPFIMTVGCVAGDEESYTVFKDLFDPIIQDRHGGFKPTDKH |
| | Kinase M-type) | KTDLNHENLKGGDDLDPHYVLSSRVRTGRSIKGYTLPPHCSRGERRAVEKLS |
| | | VEALNSLTGEFKGKYYPLKSMTEQEQQQLIDDHFLFDKPVSPLLLASGMARD |
| | | WPDARGIWHNDNKSFLVWVNEEDHLRVISMEKGGNMKEVFRRFCVGLQKIE |
| | | EIFKKAGHPFMWNEHLGYVLTCPSNLGTGLRGGVHVKLAHLSKHPKFEEILT |
| | | RLRLQKRGTGGVDTAAVGSVFDISNADRLGSSEVEQVQLVVDGVKLMVEME |
| | | KKLEKGQSIDDMIPAQK |
| 55 | Rabbit | MMMVMQPEGLGAREGPFAGGGGGGEYMEQEEDWDRDLLLDPAWEKQQRK |
| | G1U4H8 | TFTAWCNSHLRKAGTQIENIEEDFRNGLKLMLLLEVISGERLPRPDKGKMRFH |
| | (Actinin Alpha | KIANVNKALDFIASKGVKLVSIGAEEIVDGNLKMTLGMIWTIILRFAIQDISVE |
| | 3) | ETSAKEGLLLWCQRKTAPYRNVNVQNFHTSWKDGLALCALIHRHRPDLIDY |
| | | AKLRKDDPIGNLNTAFEVAEKYLDIPKMLDAEDIVNTPKPDEKAIMTYVSCFY |
| | | HAFAGAEQAETAANRICKVLAVNQENEKLMEEYEKLASELLEWIRRTVPWL |
| | | ENRVGEPSMSAMQRKLEDFRDYRRLHKPPRVQEKCQLEINFNTLQTKLRLSH |
| | | RPAFMPSEGKLVSDIANAWRGLEQVEKGYEDWLLSEIRRLQRLQHLAEKFQQ |
| | | KASLHETWTRGKEDMLTQRDYESASLQEVRALLRRHEAFESDLAAHQDRVE |
| | | HIAALAQELNELDYHEAASVNSRCQAICDQWDNLGTLTQKRRDALERMEKL |
| | | LETIDQLQLEFARRAAPFNNWLDGAIEDLQDVWLVHSVEETQSLLTAHDQFK |
| | | ATLPEADRERGAILGIQGEIQKICQTYGLRPSSTNPYINLSPQDINTKWDTVRK |
| | | LVPSRDQTLQEELARQQMNERLRRQFAAQANAVGPWIQAKVEEVGRLAAGL |
| | | AGSLEEQMAGLRQQEQNIINYKSNIDRLEGDHQLLQESLVFDNKHTVYSMEH |
| | | IRVGWEQLLTSIARTINEVENQVLTRDAKGLSQEQLNEFRASFNHFDRKRNG |
| | | MMEPDDFRACLISMYDLGEVEFARIMTMVDPNAAGVVTFQAFIDFMTRETAE |
| | | TDTAEQVVASFKILAGDKNYITPEELRRELPAEQAEYCIHRMAPYKGAGAPA |
| | | GALDYVAFSSALYGESDL |
| 56 | Rabbit PYGM | SRPLSDQEKRKQISVRGLAGVENVTELKKNFNRHLHFTLVKDRNVATPRDYY |
| | (Glycogen | FALAHTVRDHLVGRWIRTQQHYYEKDPKRIYYLSLEFYMGRTLQNTMVNLA |
| | Phosphorylase, | LENACDEATYQLGLDMEELEEIEEDAGLGNGGLGRLAACFLDSMATLGLAA |
| | muscle form) | YGYGIRYEFGIFNQKICGGWQMEEADDWLRYGNPWEKARPEFTLPVHFYGR |
| | | VEHTSQGAKWVDTQVVLAMPYDTPVPGYRNNVVNTMRLWSAKAPNDFNL |
| | | KDFNVGGYIQAVLDRNLAENISRVLYPNDNFFEGKELRLKQEYFVVAATLQD |
| | | IIRRFKSSKFGCRDPVRTNFDAFPDKVAIQLNDTHPSLAIPELMRVLVDLERLD |
| | | WDKAWEVTVKTCAYTNHTVLPEALERWPVHLLETLLPRHLQIIYEINQRFLN |
| | | RVAAAFPGDVDRLRRMSLVEEGAVKRINMAHLCIAGSHAVNGVARIHSEILK |
| | | KTIFKDFYELEPHKFQNKTNGITPRRWLVLCNPGLAEIIAERIGEEYISDLDQLR |
| | | KLLSYVDDEAFIRDVAKVKQENKLKFAAYLEREYKVHINPNSLFDVQVKRIH |
| | | EYKRQLLNCLHVITLYNRIKKEPNKFVVPRTVMIGGKAAPGYHMAKMIIKLIT |
| | | AIGDVVNHDPVVGDRLRVIFLENYRVSLAEKVIPAADLSEQISTAGTEASGTG |
| | | NMKFMLNGALTIGTMDGANVEMAEEAGEENFFIFGMRVEDVDRLDQRGYN |
| | | AQEYYDRIPELRQIIEQLSSGFFSPKQPDLFKDIVNMLMHHDRFKVFADYEEY |
| | | VKCQERVSALYKNPREWTRMVIRNIATSGKFSSDRTIAQYAREIWGVEPSRQR |
| | | LPAPDEKIP |
| 57 | Rabbit ALDOA | HSHPALTPEQKKELSDIAHRIVAPGKGILAADESTGSIAKRLQSIGTENTEENR |
| | (Fructose- | RFYRQLLLTADDRVNPCIGGVILFHETLYQKADDGRPFPQVIKSKGGVVGIKV |
| | Bisphosphate | DKGVVPLAGTNGETTTQGLDGLSERCAQYKKDGADFAKWRCVLKIGEHTPS |
| | Aldolase A) | ALAIMENANVLARYASICQQNGIVPIVEPEILPDGDHDLKRCQYVTEKVLAAV |
| | | YKALSDHHIYLEGTLLKPNMVTPGHACTQKYSHEEIAMATVTALRRTVPPAV |
| | | TGVTFLSGGQSEEEASINLNAINKCPLLKPWALTFSYGRALQASALKAWGGK |
| | | KENLKAAQEEYVKRALANSLACQGKYTPSGQAGAAASESLFISNHAY |
| 58 | Rabbit MYL1 | APKKDVKKPAAAAAPAPAPAPAPAPAPAKPKEEKIDLSAIKIEFSKEQQDEFK |
| | (Myosin Light | EAFLLYDRTGDSKITLSQVGDVLRALGTNPTNAEVKKVLGNPSNEEMNAKKI |
| | Chain, Skeletal | EFEQFLPMLQAISNNKDQGTYEDFVEGLRVFDKEGNGTVMGAELRHVLATL |
| | Muscle | GEKMKEEEVEALMAGQEDSNGCINYEAFVKHIMSI |
| | Isoform) | |
| 59 | Rabbit G3P | MVKVGVNGFGRIGRLVTRAAFNSGKVDVVAINDPFIDLHYMVYMFQYDSTH |
| | (Glyceraldehyde- | GKFHGTVKAENGKLVINGKAITIFQERDPANIKWGDAGAEYVVESTGVFTTM |
| | 3-Phosphate | EKAGAHLKGGAKRVIISAPSADAPMFVMGVNHEKYDNSLKIVSNASCTTNCL |
| | Dehydrogenase) | APLAKVIHDHFGIVEGLMTTVHAITATQKTVDGPSGKLWRDGRGAAQNIIPAS |
| | | TGAAKAVGKVIPELNGKLTGMAFRVPTPNVSVVDLTCRLEKAAKYDDIKKV |
| | | VKQASEGPLKGILGYTEDQVVSCDFNSATHSSTFDAGAGIALNDHFVKLISWY |
| | | DNEFGYSNRVVDLMVHMASKE |
| 60 | Rabbit TPM1 | MDAIKKKMQMLKLDKENALDRAEQAEADKKAAEDRSKQLEDELVSLQKKL |
| | (Tropomyosin | KGTEDELDKYSEALKDAQEKLELAEKKATDAEADVASLNRRIQLVEEELDRA |
| | Alpha-1 Chain) | QERLATALQKLEEAEKAADESERGMKVIESRAQKDEEKMEIQEIQLKEAKHI |
| | | AEDADRKYEEVARKLVIIESDLERAEERAELSEGKCAELEEELKTVINNLKSL |
| | | EAQAEKYSQKEDKYEEEIKVLSDKLKEAETRAEFAERSVTKLEKSIDDLEDEL |
| | | YAQKLKYKAISEELDHALNDMTSI |
| 61 | Rabbit Myosin- | EAKPAARKAPKGKDAPKEEPPKEAAAEPPKEAPPEDQSPTAEEPTGIFLKKPD |
| | Binding protein | SVSVETGKDAVIMAKLNGKELPAKPTIKWFKGKWLELGSKSGARFSFKETHD |
| | C (fast-type) | AASNVYSLELHIGKVVLGDRGDYRLEVKAKDVCDSCSFNIDVEAPRQDSAGQ |
| | | SLESFKRAGDAKPDGAGELDFSGLLKKREVVEEEKKKKKDDDDLGIPPEIWE |
| | | LLKGAKKSEYERIAFQYGITDLRGMLKRLKKAKVEVKKSAAFTKKLDPAYQ |
| | | VDRGNKVKLVVEISDPDLPLKWFKNGQEIKPSLKYVFENVGKKRILTINKCTL |
| | | ADDAAYEVVVNDEKCFTELFVKEPPVLIVTPLEDQQVFVGDRVEMSVEVSEE |
| | | GAQVMWMKDGVELTREGSFKSNYRFKKDGKRHILIYSEVNLEDRGHYQVM |
| | | TNGGQCEAELIVEEKQLEVLQDIADLTVKASEQAMFKCEVSDEKVTGKWYK |
| | | NGVEVRPSKRITISHVGRFHKLVIDDVRPEDEGDYTFVPDGYALSLSAKLNFL |
| | | EIKVEYVPKQEPPKIHLDCSGKTSDNAIVVVAGNKLRLDVSITGEPPPVATWM |
| | | KGDEVFTVTEGRVRIEQRPDSSSFVIESAERGDEGRYTIKVTNPAGEDVATVM |
| | | LRVVDVPDPPEAVRVTSVGEDWAILVWDPPKYDGGQPVTGYLLERKKKGSQ |
| | | RWMKLNFEVFTETTYESTKMIEGILYEMRVFAVNAIGVSQPSMNTKPFMPIAP |
| | | TSAPQHLTVEDVTDTTTTLKWRPPDRIGAGGIDGYLVEYCVEGSDEWIPANK |
| | | EPVERCGFTVKGLPTGAKILFRVIGVNIAGRSEPATLVQPVTIREIVEQPKIRLP |
| | | RHLRQTYVRKVGEHINLVIPFQGKPRPQVVWTKGGAPVDTSRVHVRTSDFDT |
| | | VFFVREAARSDSGEYELSVQIENMKDTATVRIRVVEKAGPAENVMVKEVWG |
| | | TNALVEWQPPKDNGNSEITGYLVQKADKKTMEWFTVYEHNRHTSCTVSDLI |
| | | VGNEYYFRVYTENVCGLSDSPGVSKNTAHILKTGITFKPFEYKEHDFRSPPKF |
| | | LTPLIDRVVVAGYAAALNCAVRGHPKPKVVWMKNKVEIHEDPKFLMTNHQ |
| | | GVLTLNIRRPSPFDAGTYSCRAFNELGEALAECRLDVRVPQ |
| 62 | Rabbit G1T4T6 | MSLVAVPFYQKRHKHFDQSYRNIQTKYLMDEYTAKKRASTQMSTHKSASTQ |
| | (Myomesin 2) | MATHKSALQKASSQTSSGGTTCRLCAKRLSAAEEEEQESKNRYRSLVASYGE |
| | | AKRQRFLSELAQLEESVHLARSQARDVVDKYALQQMVEDKMAWERHVFEE |
| | | RLSRAPEILVQLRSHTVWEKMSVKLCFTVQGFPTPVVQWYKDGSLICQAGEP |
| | | GKYRIDSKYGVHTLEISRASFDDTATYSAVATNAHGQVSTNAAVVVRRYRG |
| | | DEEPFYSVGLPIGYVLVKESKWTRMFFGEGQASLSFSHLNKDDEGLYTLRIVS |
| | | RGGVSDHSAFLFVRDADPLVTGAPGAPMDLQCHDANRDYVIVTWKPPNTTT |
| | | ESPVIGYFVDRCEVGTNNWVQCNDAPVKICKYPVTGLYEGRSYIFRVRAVNS |
| | | AGISRPSRVSDAVAALDPADLRRLQAIHLEGEKEVVIYQDDLEGDVQVPGPPT |
| | | NVHASEISRNYVVLSWEPPVPRGKEPLMYFIEKSVVGSGSWQRVNAQTAVRS |
| | | PRYAVFDLAEGKPYVFRVLSANKHGLSDPSEITSPIQAQDTIAVPSAPGRVLAS |
| | | RNTKTSVVVQWDRPKHEEDLLGYYVDCCVAGTNAWEPCNHKPIGYNRFVV |
| | | HGLTTGEQYIFRVKAVNALGTSENSQESEVIKVQAALTVPSHPYGVTLLNCD |
| | | GHSMTLGWKVPKFSGGSPILGYYVDKREVHHKNWHEVNSSPIQERILTVEGL |
| | | TEGSFYEFKIAATNLAGIGQPSDPSEHFKCEAWTAPEPGPAYDLTFCEVRDTSL |
| | | VMLWKAPVYSGSSPVSGYFVDFKEEDSGEWSTVNEAATPNRYLKVSDLHQG |
| | | RTYVFRVRAVNASGVGKPSDVSEPVLVEARPGTKEISAGVDEEGNIYLGFDC |
| | | QEMTDASHFTWCKSYEEIADPERFHVETKGDHSKLYFKNPDKADLGTYSVSV |
| | | SDTDGVSSSFVLDEEELERLLALSNEIKNPTIPLKSELAYEIFDKGQVRFWLQT |
| | | EPLSPDASFRFVINDREVSDSETHRMKCDKASGIIEMVMDRFTIENEGTYTVQI |
| | | HDGKAKNQSSLVLIGDAFRKVLEEAEFQRKEFLRKQGPHFAEYLHWDVTEEC |
| | | EVRLICKVANTKKETVFKWLKDDVLYETETPPDLEKGVCELLIPKLSKKDHG |
| | | EYKATLKDERGQDASILEIAGKVYEDMILAMSRVCGMSASPLKILCTPEGIRL |
| | | QCFMKYFTEEMRVNWYHKDAKISSSEHMRIGGSEDMAWLQICEPTEKDKGK |
| | | YTFEIFDGKDNHQRSLDLSGQAFDEAFAEFQRLKAAAFAEKNRGKVIGGLPD |
| | | VVTIMEGKTLNLTCTVFGNPDPEVVWFKNDQDIELSEHFSVKVEQAKYVSLTI |
| | | KGVTSEDSGKYSINVKNKYGGEKIDVTVSVYKHGEAIPEVAPPQQAKPKLIPA |
| | | SASATAP |
| 63 | Rabbit ENOB | AMQKIFAREILDSRGNPTVEVDLHTAKGRFRAAVPSGASTGIYEALELRDGDK |
| | (Beta-enolase) | SRYLGKGVLKAVEHINKTLGPALLEKKLSVVDQEKVDKFMIELDGTENKSKF |
| | | GANAILGVSLAVCKAGAAEKGVPLYRHIADLAGNHDLVLPVPAFNVINGGSH |
| | | AGNKLAMQEFMILPVGASSFREAMRIGAEVYHHLKGVIKAKYGKDATNVGD |
| | | EGGFAPNILENNEALELLKTAIQAAGYPDKVVIGMDVAASEFHRNGKYDLDF |
| | | KSPDDPARHITGQKLGELYKSFIKNYPVVSIEDPFDQDDWATWTSFLSGVDIQI |
| | | VGDDLTVTNPKRIAQAVEKKACNCLLLKVNQIGSVTESIQACKLAQSNGWGV |
| | | MVSHRSGETEDTFIADLVVGLCTGQIKTGAPCRSERLAKYNQLMRIEEALGD |
| | | KAVFAGRKFRNPKAK |
| 64 | Rabbit MLRS | APKKAKRRAAAEGGSSNVFSMFDQTQIQEFKEAFTVIDQNRDGIIDKEDLRDT |
| | (Myosin | FAAMGRLNVKNEELDAMMKEASGPINFTVFLTMFGEKLKGADPEDVITGAF |
| | Regulatory | KVLDPEGKGTIKKQFLEELLTTQCDRFSQEEIKNMWAAFPPDVGGNVDYKNI |
| | Light Chain 2, | CYVITHGDAKDQE |
| | Skeletal Muscle | |
| | Isoform Type 2) | |
| 65 | Truncation of | IFSISDNAYQFMLTDRENQSILITGESGAGKTVNTKRVIQYFATIAITGDKKKEE |
| | Rabbit G1SJN7 | PTPGKMQGTLEDQIISANPLLEAFGNAKTVRNDNSSRFGKFIRIHFGATGKLAS |
| | (Myosin 4) | ADIETYLLEKSRVTFQLKAERSYHIFYQIMSNKKPELIEMLLITTNPYDFAFVS |
| | | QGEITVPSIDDQEELMATDSAVDILGFTSDEKVAIYKLTGAVMHYGNLKFKQ |
| | | KQREEQAEPDGTEVADKAAYLTSLNSADLLKALCYPRVKVGNEYVTKGQTV |
| | | QQVYNAVGALAKAIYEKMFLWMVTRINQQLDTKQPRQYFIGVLDIAGFEIFD |
| | | FNSLEQLCINFTNEKLQQFFNHHMFVLEQEEYKKEGIEWEFIDFGMDLAACIE |
| | | LIEKPMGIFSILEEECMFPKATDTSFKNKLYEQHLGKSNNFQKPKPAKGKAEA |
| | | HFSLVHYAGTVDYNITGWLDKNKDPLNETVVGLYQKSGMKTLAFLFSGAQT |
| | | GEEGGGGGKKGGKKKGSSFQTVSALFRENLNKLMTNLRSTHPHFVRCLIPNE |
| | | TKTPGAMEHELVLHQLRCNGVLEGIRICRKGFPSRILYADFKQRYKVLNASAI |
| | | PEGQFIDSKKASEKLLGSIDVDHTQYKFGHTKVFFKAGLLGLLEEMRDEKLA |
| | | QLITRTQAICRGYLTRVEFKKMMERRESIFCIQYNIRAFMNVKHWPWMKLYF |
| | | KIKPLLKSAETEKEMANMKEEFEKTKEELAKSEAKRKELEEKMVALMQEKN |
| | | DLQLQVQAEADGLADAEERCDQLIKTKIQLEAKIKEVTERAEDEEEINAELTA |
| | | KKRKLEDECSELKKDIDDLELTLAKVEKEKHATENKVKNLTEEMAGLDENIA |
| | | KLTKEKKALQEAHQQTLDDLQAEEDKVNTLTKAKTKLEQQVDDLEGSLEQE |
| | | KKLRMDLERAKRKLEGDLKLAQESTMDIENDKQQLDEKLKKKEFEMTNLQS |
| | | KIEDEQALAMQLQKKIKELQARIEELEEEIEAERASRAKAEKQRSDLSRELEEI |
| | | SERLEEAGGATSAQIEMNKKREAEFQKMRRDLEEATLQHEATAAALRKKHA |
| | | DSVAELGEQIDNLQRVKQKLEKEKSELKMEIDDLAGNMETVSKAKGNLEKM |
| | | CRTLEDQLSEVKTKEEEHQRLINELSAQKARLHTESGEFSRQLDEKDAMVSQ |
| | | LSRGKQAFTQQIEELKRQLEEETKAKSALAHALQSSRHDCDLLREQYEEEQE |
| | | AKAELQRAMSKANSEVSQWRTKYETDAIQRTEELEEAKKKLAQRLQDAEEH |
| | | VEAVNSKCASLEKTKQRLQN |
| 66 | Truncation of | IFYQIMSNKKPELIEMLLITTNPYDFAFVSQGEITVPSIDDQEELMATDSAVDIL |
| | Rabbit G1SJN7 | GFTSDEKVAIYKLTGAVMHYGNLKFKQKQREEQAEPDGTEVADKAAYLTSL |
| | (Myosin 4) | NSADLLKALCYPRVKVGNEYVTKGQTVQQVYNAVGALAKAIYEKMFLWM |
| | | VTRINQQLDTKQPRQYFIGVLDIAGFEIFDFNSLEQLCINFTNEKLQQFFNHHM |
| | | FVLEQEEYKKEGIEWEFIDFGMDLAACIELIEKPMGIFSILEEECMFPKATDTSF |
| | | KNKLYEQHLGKSNNFQKPKPAKGKAEAHFSLVHYAGTVDYNITGWLDKNK |
| | | DPLNETVVGLYQKSGMKTLAFLFSGAQTGEEGGGGGKKGGKKKGSSFQTVS |
| | | ALFRENLNKLMTNLRSTHPHFVRCLIPNETKTPGAMEHELVLHQLRCNGVLE |
| | | GIRICRKGFPSRILYADFKQRYKVLNASAIPEGQFIDSKKASEKLLGSIDVDHT |
| | | QYKFGHTKVFFKAGLLGLLEEMRDEKLAQLITRTQAICRGYLTRVEFKKMME |
| | | RRESIFCIQYNIRAFMNVKHWPWMKLYFKIKPLLKSAETEKEMANMKEEFEK |
| | | TKEELAKSEAKRKELEEKMVALMQEKNDLQLQVQAEADGLADAEERCDQLI |
| | | KTKIQLEAKIKEVTERAEDEEEINAELTAKKRKLEDECSELKKDIDDLELTLAK |
| | | VEKEKHATENKVKNLTEEMAGLDENIAKLTKEKKALQEAHQQTLDDLQAEE |
| | | DKVNTLTKAKTKLEQQVDDLEGSLEQEKKLRMDLERAKRKLEGDLKLAQES |
| | | TMDIENDKQQLDEKLKKKEFEMTNLQSKIEDEQALAMQLQKKIKELQARIEE |
| | | LEEEIEAERASRAKAEKQRSDLSRELEEISERLEEAGGATSAQIEMNKKREAEF |
| | | QKMRRDLEEATLQHEATAAALRKKHADSVAELGEQIDNLQRVKQKLEKEKS |
| | | ELKMEIDDLAGNMETVSKAKGNLEKMCRTLEDQLSEVKTKEEEHQRLINELS |
| | | AQKARLHTESGEFSRQLDEKDAMVSQLSRGKQAFTQQIEELKRQLEEETKAK |
| | | SALAHALQSSRHDCDLLREQYEEEQEAKAELQRAMSKANSEVSQWRTKYET |
| | | DAIQRTEELEEAKKKLAQRLQDAEEHVEAVNSKCASLEKTKQRLQNEVEDL |
| | | MIDVERSNAACAALDK |
| 67 | Truncation of | TLDDLQAEEDKVNTLTKAKTKLEQQVDDLEGSLEQEKKLRMDLERAKRKLE |
| | Rabbit G1SJN7 | GDLKLAQESTMDIENDKQQLDEKLKKKEFEMTNLQSKIEDEQALAMQLQKKI |
| | (Myosin 4) | KELQARIEELEEEIEAERASRAKAEKQRSDLSRELEEISERLEEAGGATSAQIE |
| | | MNKKREAEFQKMRRDLEEATLQHEATAAALRKKHADSVAELGEQIDNLQRV |
| | | KQKLEKEKSELKMEIDDLAGNMETVSKAKGNLEKMCRTLEDQLSEVKTKEE |
| | | EHQRLINELSAQKARLHTESGEFSRQLDEKDAMVSQLSRGKQAFTQQIEELKR |
| | | QLEEETKAKSALAHALQSSRHDCDLLREQYEEEQEAKAELQRAMSKANSEVS |
| | | QWRTKYETDAIQRTEELEEAKKKLAQRLQDAEEHVEAVNSKCASLEKTKQR |
| | | LQNEVEDLMIDVERSNAACAALDKKQRNFDKVLAEWKHKYEETQAELEASQ |
| | | KESRSLSTEVFKVKNAYEESLDHLETLKRENKNLQQEISDLTEQIAESGKHIHE |
| | | LEKVKKQIDQEKSELQAALEEAEGSLEHEEGKILRIQLELNQVKSEIDRKIAEK |
| | | DEEIDQLKRNHLRVVESMQSTLDAEIRSRNDALRIKKKMEGDLNEMEIQLNH |
| | | ANRQAAEAIKNLRNTQGILKDTQLHLDDAVRGQDDLKEQLA |
| 68 | Truncation of | LAQESTMDIENDKQQLDEKLKKKEFEMTNLQSKIEDEQALAMQLQKKIKELQ |
| | Rabbit G1SJN7 | ARIEELEEEIEAERASRAKAEKQRSDLSRELEEISERLEEAGGATSAQIEMNKK |
| | (Myosin 4) | REAEFQKMRRDLEEATLQHEATAAALRKKHADSVAELGEQIDNLQRVKQKL |
| | | EKEKSELKMEIDDLAGNMETVSKAKGNLEKMCRTLEDQLSEVKTKEEEHQR |
| | | LINELSAQKARLHTESGEFSRQLDEKDAMVSQLSRGKQAFTQQIEELKRQLEE |
| | | ETKAKSALAHALQSSRHDCDLLREQYEEEQEAKAELQRAMSKANSEVSQWR |
| | | TKYETDAIQRTEELEEAKKKLAQRLQDAEEHVEAVNSKCASLEKTKQRLQNE |
| | | VEDLMIDVERSNAACAALDKKQRNFDKVLAEWKHKYEETQAELEASQKESR |
| | | SLSTEVFKVKNAYEESLDHLETLKRENKNLQQEISDLTEQIAESGKHIHELEKV |
| | | KKQIDQEKSELQAALEEAEGSLEHEEGKILRIQLELNQVKSEIDRKIAEKDEEI |
| | | DQLKRNHLRVVESMQSTLDAEIRSRNDALRIKKKMEGDLNEMEIQLNHANR |
| | | QAAEAIKNLRNTQGILKDTQLHLDDAVRGQDDLKEQLAMVERRANLMQAEI |
| | | EELRASLEQTERSRRVAEQELLDASERVQLLHTQNTSLINTKKKLETDISQIQG |
| | | EMEDIVQEARNAEEKAKKAITDAAMMAEELKKEQDTSAHLERMKKNMEQT |
| | | VKDLQHRLDEAEQLALKGGKKQIQKLEARVRELENEVESEQKRNVEAVKGL |
| | | RKHERRVKELTYQTEEDRKNVLRLQDLVDKLQSKVKAYKRQAEEAEEQSN |
| 69 | Truncation of | VFVADPKESFVKATVQSREGGKVTAKTEAGATVTVKEDQVFPMNPPKYDKI |
| | Rabbit MYH4 | EDMAMMTHLHEPAVLYNLKERYAAWMIYTYSGLFCVTVNPYKWLPVYNAE |
| | (Myosin 4) | VVTAYRGKKRQEAPPHIFSISDNAYQFMLTDRENQSILITGESGAGKTVNTKR |
| | | VIQYFATIAVTGDKKKEEATSGKMQGTLEDQIISANPLLEAFGNAKTVRNDNS |
| | | SRFGKFIRIHFGTTGKLASADIETYLLEKSRVTFQLKAERSYHIFYQIMSNKKP |
| | | DLIEMLLITTNPYDYAFVSQGEITVPSIDDQEELMATDSAIDILGFTSDERVSIY |
| | | KLTGAVMHYGNMKFKQKQREEQAEPDGTEVADKAAYLQSLNSADLLKALC |
| | | YPRVKVGNEYVTKGQTVQQVYNAVGALAKAVYEKMFLWMVTRINQQLDT |
| | | KQPRQYFIGVLDIAGFEIFDFNSLEQLCINFTNEKLQQFFNHHMFVLEQEEYKK |
| | | EGIEWEFIDFGMDLAACIELIEKPMGIFSILEEECMFPKATDTSFKNKLYEQHL |
| | | GKSNNFQKPKPAKGKVEAHFSLVHYAGTVDYNITGWLDKNKDPLNETVVGL |
| | | YQKSAMKTLAFLFTGTAAAEAEGGGKKGGKKKGSSFQTVSALFRENLNKLM |
| | | TNLRSTHPHFVRCIIPNETKTPGAMEHELVLHQLRCNGVLEGIRICRKGFPSRIL |
| | | YADFKQRYKVLNASAIPEGQFIDSKKASEKLLGSIDVDHTQYKFGHTKVFFKA |
| | | GLLGLLEEMRDDKLAQLITRTQAMCRGFLARVEYKKMVERRESIFCIQYNIR |
| | | AFMNVKHWPWMKLYFKIKPLLKSAETEKEMANMKEEFEKTKESLAKAEAK |
| | | RKELEEKMVALMQEKNDLQLQVQAEADSLADAEERCDQLIKTKIQLEAKIKE |
| | | VTERAEDEEEINAELTAKKRKLEDECSELKKDIDDLELTLAKVEKEKHATENK |
| | | VKNLTEEMAGLDETIAKLTKEKKALQEAHQQTLDDLQAEEDKVNTLTKAKT |
| | | KLEQQVDDLEGSLEQEKKIRMDLERAKRKLEGDLKLAQESTMDIENDKQQL |
| | | DEKLKKKEFEMSNLQSKIEDEQALAMQLQKKIKELQARIEELEEEIEAERASR |
| | | AKAEKQRSDLSRELEEISERLEEAGGATSAQIEMNKKREAEFQKMRRDLEEA |
| | | TLQHEATAATLRKKHADSVAELGEQIDNLQRVKQKLEKEKSELKMEIDDLAS |
| | | NMETVSKAKGNLEKMCRTL |
| 70 | Truncation of | TLVSDVDYRNYLHQWTCMPDQNDVIQAKKAYELQSDAVYKADLEWLRGIG |
| | Rabbit Nebulin | WMPSDSVSVNHAKHAADIFSEKKYRTKIETLNFTPVDDRVDYVTAKQSGEIL |
| | | DDIKYRKDWNSIKSKYTLTETPLLHTVQEAARILDQYLYKESWEKQKATGYI |
| | | LPPDAVPFVHAHHSSDVQSELKYKAEHVKQRGHYVGVPTMRDDPKLVWFE |
| | | HAGQIQNDRLYKEDYHKTKAKINIPADMVSVVAAKEGQNLVSDIDYRNYLH |
| | | QWTCHPDQNDVIQARKAYDLQSDNVYKADLEWLRGIGWIPLDSVDHVRVT |
| | | KNQEMVNQIKYKKDALDNYPNFTSVVDPPAIVLAKINAVNQSDVKYKETFN |
| | | KAKGKYTFSPDTPHISHSKDMGKLYSTILYKGAWEGTKAYGYTLDERYIPIVG |
| | | AKHADFVNSELKYKETYEKQKGHYLAGKVISEFPGVVHCLDFQKMRSVLNY |
| | | RRDYENTKANVHIPNDMMNHVLAKKCQYILSDLEYRHYFHQWTSLPDEPKV |
| | | LLARNAQEILSDNVYKDDLNWLKGIGCYVWDTPQILHAKKSYDLQSQLQYT |
| | | AAGKENLQNYNLVTDTPLYVTALQSGINASEVKYKENYHQIKDKYTTVLETV |
| | | DYDRTRNLKDLYSSNLYKEAWNKVKATSYILPTSTLSLTHAKNQKHLASHIK |
| | | YREEYEKFKALYTLPKSVEDDPNTARCLRVGKLNIDRLYRSVYEKNKMKIHI |
| | | VPDMVEMVTAKDTQKKVSEIDYRLHLHEWICHPDLQVNSHVRKVTDQISDIV |
| | | YKDDLNWLKGIGCYVWDTPEILHAKHAYDLRNDIKYKAHMMKTRNDYKLV |
| | | TDTPVYVQAVKSGKQLSDAVYHHDYVHSVRGKVAPTTKTVDLERALHAYK |
| | | LQSENLYKTSLRTLPTGYRLPGDTPHFKHNKDIRYMSSYFKYKEVYEHIKAN |
| | | GYTLGPNDVPFVNVRRVNKVTSERLYRELYHKLKDKIHTTPDTPEIRQVKKT |
| | | QEAVSELIYKSDFFKMQGHMISLPYTPHVLHCRYVGDITSDIKYKEDLQILKG |
| | | LGCFLYDTPDMVRSRHLRKLWSNYLYTDNARKMRDKYKVVLDTPEYRKVQ |
| | | ELKTHLSELVYRASGRKQKSIFTSVPDTPDLLRAKRGQKLQSQYLYVELATKE |
| | | RPHHHAGNQTTALKHARDVKDMVSEKKYKVQYEKMKDKYTPIPDTPILIRA |
| | | KNAYWNASDLRYKETFQKTKGKYHTVKDALDIVYHRKVTDDISKVKYKEN |
| | | YMRHLGIWRSIPDRPEHFHHRAVTDAVSDIKYKEDLTWLKGIGCYAYDTPDF |
| | | TLAEKNKTLYSKYKYKEVFERTKSDFKYVADCPINRHFKYATQLMNEKKYR |
| | | ADYEQRKDKYQLVVDEPRHLLAKTAGDQISQIKYRKQYEKSKDKFTSIVDTP |
| | | EHLRTTKVNKQISDILYKLEYNKAKPRGYTTIHDTPMLLHVRKVKDEVSDLK |
| | | YKEVYQRNKSNCTIKPDAVHIKAAKDAYKVNTNLDYKKQYEANKAHWRWT |
| | | PDRPDFIQAAKSSLQQSDFEYKLDREFLKGCKLSVTDDKDMVLALKNSLIESD |
| | | LKYKEKHVKERGSCHAVPDTPQILLAKTVSNLV |
| 71 | Truncation of | EHTRNVNQIQSDNVYKDEYNNFFKGIGWIPIGSLEVEKVKKAGDALNEKKYR |
| | Rabbit Nebulin | QHPDTIKFTSVPDSMGMVLAQHNTKQLSDLNYKVEGEKVKHKYTMDPEVPQ |
| | | FIQARVNAFNLSDAHYKADWKKTLAKGYDLRPDAIPIVAAKSSRNIASDCKY |
| | | KEAYEKAKGKQIGFLSLQDDPKLVHYMNVAKIQSDREYRKGYEASKTRYHT |
| | | PLDMLSVTAAKKSQEVATNANYKQPFHNYTLLPDALPVEHSRNAMQIQSDN |
| | | LYKSDFTGWMKGIGWIPIQSLEVEKAKKAGEILSEKRYRQHPEKLKFTYAMD |
| | | TMEQALNKANKLNMDKRLYTKKWDKDKTTIHVMPDTPDILLSRVNQITMSD |
| | | KLYRAGWEEEKKKGYDLRPDAISIKAAKASRDIASDYKYKQAYEQAKGKHI |
| | | GFRSLEDDPKLVHFMQVAKMQSDREYKKAYEKSKTSFHTPVDMLSVVAAK |
| | | KSQEVATNANYRNVIHTYNMLPDAMGFELAKNMMQIQSDNQYKADYADFM |
| | | KGIGWLPLGSLEAEKNKKAMEIISEKKYRQHPDTLKYSTLMDSMNMVLAQN |
| | | NAKIMNEHLYKQAWEADKTKVHIMPDIPQIVLAKANAINMSDKLYKLSMEE |
| | | AKKKGYDLRTDAIPIKAAKASRDIASDYKYKYNYEKQKGKMVGFRSLEDDP |
| | | KLVHSMQVAKMQSDREYKKNYEKTKTSYHTPADMLSVTAAKDAQANITNT |
| | | NYKHLIHKYILLPDAMSLELSRNMNRIQSDNEYKQDYNEWYKGLGWSPAGS |
| | | LDVEKAKKATEYASDQKYRQHPSNFQFKKLTDSMDMVLAKQNAHTMNKHL |
| | | YTIDWNKDKTKIHVMPDTPDILQAKQNQTLFSQKFYKLGWEEALKKGYDLP |
| | | VDAISVQLAKASRDIASDFKYKQGYRKQLGHHVGFRSLQDDPKLVLSMNVA |
| | | KMQSEREYKKDFEKWKTKFSSPVDMLGVVLAKKCQALVSDVDYKNYLHQ |
| | | WTCLPDQNDVIHAKKAYELQSENMYKSDLEWLKGIGWSPLGSLEAEKNKRA |
| | | SEILSEKKYRQPPDTNKFTSIPDAMDIVLAKTNAKNRSDRLYREAWDKDKTQ |
| | | VHIMPDTPDIILAKANLINTSDKLYRMGYEELKKKGYDLPVDAIPIKAAKASR |
| | | EIASEYKYKEGFRKQLGHHIGARNIKDDPKMMWSMHVAKIQSDREYKKDFE |
| | | KWKTKFSSPVDMLGVVLAKKCQTLVSDIDYKNYLHQWTCLPDQNDVTHAR |
| | | HAYDLQSDNLYKSDLQWLKGIGWIPTGSIEDEKNKRATQILSDHVYRQHPDK |
| | | FKFSILMDSIPMVLAKNNATNMNHRLYTEAWDKDKTTVHIMPDTPEVLLAK |
| | | QNQVNCSEKLYKLGLEETKRKGYDMRVDAIPIKAAKASRDIASEFKYKEGYR |
| | | KQLGHHIGARAIHDDPKMMWSMHVAKIQSDREYKKDFEKWKTKFSSPVDM |
| | | LGVVLAKKCQALVSDVDYKNYLHQWTCLPDQNDVMHARQAYDLQSDNLY |
| | | KSDLQWLRGIGWVPIGSLDVEKC |
| 72 | Truncation of | DVTHARHAYDLQSDNLYKSDLQWLKGIGWIPTGSIEDEKNKRATQILSDHVY |
| | Rabbit Nebulin | RQHPDKFKFSILMDSIPMVLAKNNATNMNHRLYTEAWDKDKTTVHIMPDTP |
| | | EVLLAKQNQVNCSEKLYKLGLEETKRKGYDMRVDAIPIKAAKASRDIASEFK |
| | | YKEGYRKQLGHHIGARAIHDDPKMMWSMHVAKIQSDREYKKDFEKWKTKF |
| | | SSPVDMLGVVLAKKCQALVSDVDYKNYLHQWTCLPDQNDVMHARQAYDL |
| | | QSDNLYKSDLQWLRGIGWVPIGSLDVEKCKKATEILSDKIYRQPPDRFKFTSV |
| | | TDSLEQVLAKNNAINMNKRLYTEAWDKDKTQIHIMPDTPEIMLARMNKINYS |
| | | ESLYKLANEEAKKKGYDLRSDAIPIVAAKASRDIASDYKYKDGYRKQLGHHI |
| | | GARDIKDDPKMMWSMHVAKIQSDREYKKDFEKWKTKFSSPVDMLGVVLAK |
| | | KCQTLVSDIDYKNYLHQWTCLPDQNDVMHARQAYDLQSDNIYKSDLQWLR |
| | | GIGWVPIGSLEVAKCKRAAEILSDNIYRQPPDKLKFTSVPDSLEQVLAKNNAIN |
| | | MNKRLYTEAWDKDKTQIHIMPDTPEIMLARQNKINYSESLYKLANEEAKKKG |
| | | YDLRSDAIPIVAAKASRDIASDYKYKDGYRRQLGHHIGARDIKDDPKMMWS |
| | | MHVAKIQSDREYKKDFEKWKTKFSSPVDMLGVVLAKKCQTLVSDIDYKNYL |
| | | HQWTCLPDQNDVMHARQAYDLQSDNIYKSDLQWLRGIGWVPIGSVDLVKC |
| | | KRAAEILSDNIYRQSPDKLKFTSVTDSLEQVLAKNNAINMNKRLYTEAWDKD |
| | | KTQIHMMPDTPEIMLARQNKMNYSENFYRQAMEEAKKEGYDLRSDAIPIVA |
| | | AKASRDIASDYKYKEAYRKQLGHHIGARAIHDDPKMMWSLHIAKVQSDREY |
| | | KKEFEKYKTRYSSPVDMLGIVLAKKCQTLVSDVDYKNLLHEWTCLPDQNDV |
| | | IQARKAYDLQSDNLYKSDLEWLRGIGWVPIGSLEVVKAKRATEILSDNLYRT |
| | | RPDTLKFTSVTDSLEQVLAKNNAINMNKRLYTEAWENDKKTIHVMPDTPEIM |
| | | LAKLNKINYSDKLYKLALEESKKEGYDLRVDAIPIQAAKASRDIASDYKYKE |
| | | GYRKQLGHHIGARNIKDDPKMMWSMHVAKIQSDREYKKDFEKWKTKFSSP |
| | | VDMLGVVLAKKCQILVSDIDYKHPLHEWICLPDQNDVIQARKAYDLQSDAV |
| | | YKADLEWLRGIGWVPIGSVEVEKVKRAGEILSDRKYRQPADKLKFTSITDTPE |
| | | IVLAKSNALTMSKHLYTEAWDADKTSIHVMPDTPEILLAKSNSANISQKLYTK |
| | | GWDESKMKDYDLKADAIPIKSAKASRDIASDYKYKEAYEKQKGHHIGAQSIE |
| | | DDPKIMCAIHA |
| 73 | Truncation of | DKRETSRPNWAQVSANVPITSCSVEKLIEGHEYQFRICAENKYGVGDPVFTEP |
| | Rabbit TTN | AIAKNPYDPPGRCDPPVISNVTKDHMTVSWKPPADDGGSPITGYLLEKRETHA |
| | | VNWTKVNRKPVIERTIKATGLQEGTEYEFRVTAINKAGPGKPSDASKAVYAQ |
| | | DPLYPPGPPAFPKVYDTTRSSVSLSWGKPAYDGGSPIIGYLVEVKRADTDNW |
| | | VRCNLPQKLQKTRFEVTGLMENTEYQFRVYAVNKVGYSDPSDVPDKHCPKD |
| | | ILIPPEGELDADLRKTLILRAGVTMRLYVPVKGRPPPKITWSKPNVNLRERIGL |
| | | DIKSTDFDTFLRCENVNKYDAGKYILTLENSCGKKEYTIVVKVLDTPGPPVNV |
| | | TVKEISRDSAYITWDPPIVDGGSPIINYVVEKRDAERKSWSTVTTECSKTSFRV |
| | | SNLEEGKSYFFRVFAENEYGIGDPGETRDAVKASETPGPVVDLKVLTVTKSSC |
| | | NIGWKKPRSDGGSRITGYVVDFLTEENKWQRVMKSLSLQYSTKDLNEGKQY |
| | | TFRVSAENENGEGTPSEITVVAKDDVVAPDLDLKDLPDLCYLAKENSNFRLKI |
| | | PIKGKPAPSVTWKKGEDPLATDTRVSVESSAVNTTLVVYDCQKSDAGKYTIT |
| | | LKNVAGTKEGTLSIKVVGKPGIPTGPIKFDEVTAEAITLKWGPPKDDGGSEITN |
| | | YILEKRDSVNNKWVTCASAVQKTTFRVTRLHEGMEYTFRVSAENKYGVGEG |
| | | LKSEPIVAKHPFDVPDAPPPPNIVDVRHDSVSLTWTDPRKTGGSPITGYHIEFK |
| | | ERNSLLWKRANKTPIRMKDFKVTGLTEGLEYEFRVMAINLAGVGKPSLPSEP |
| | | VVALDPIDPPGKPEVINVTRNSVTLIWTEPKYDGGHKLTGYIVEKRDLPSKTW |
| | | MKANHINVPDCAFTVTDLVEGGKYEFRIRAKNTAGAISAPSESTGTIICKDEYE |
| | | APTIVLDPTIKDGLTIKAGDTIVLNAISILGKPLPKSSWSKAGKDIRPSDITQITS |
| | | TPTSSMLTVKYASRKDAGEYTITATNPFGTKEEHVRVTVLDVPGPPGPIEISNV |
| | | SAEKATLTWTPPLEDGGSPIKSYVLEKRETSRLLWTVVAEDIQSCRHVVTKLI |
| | | QGNEYLFRVSAVNHYGKGEPVQSEPVKMVDRFGPPGPPGKPEVSNVTKNTA |
| | | TVSWKRPTDDGGSEITGYYVERREKKGLRWVRATKTPVSDLRCKVTGLQEG |
| | | NTYEFRVSAENRAGIGPPSDASNYVLMKDVAYPPGPPSNARVTDTTKKSASL |
| | | AWGKPHYDGGLEITGYVVEHQKVGDETWVKDTTGPALRITEFVVPDLHTKE |
| | | KYNFRISAINDAGVGEPAVIPDVEIVEREMAPDFELDAELRRTLVVRAGLSIRI |
| | | FVPIKGRPAPEVTWTKDDINLKTRANIENTESFTLLIIPECNRYDTGKFVMTIEN |
| | | PAGKKSGFVNVRVLDTPGPVLNLRPTDITKDSVTLHWDLPLIDGGSRITNYIV |
| | | EKREATRKSYSTVTTKCHKCT |
| 74 | Truncation of | DKETPSGFTLDDVIQTGVDNPGHPFIMTVGCVAGDEESYTVFKDLFDPIIQDR |
| | Rabbit KCRM | HGGFKPTDKHKTDLNHENLKGGDDLDPHYVLSSRVRTGRSIKGYTLPPHCSR |
| | (Creatine | GERRAVEKLSVEALNSLTGEFKGKYYPLKSMTEQEQQQLIDDHFLFDKPVSPL |
| | Kinase M- | LLASGMARDWPDARGIWHNDNKSFLVWVNEEDHLRVISMEKGGNMKEVFR |
| | Type) | RFCVGLQKIEEIFKKAGHPFMWNEHLGYVLTCPSNLGTGLRGGVHVKLAHLS |
| | | KHPKFEEILTRLRLQKRGTGGVDTAAVGSVFDISNADRLGSSEVEQVQLVVD |
| | | GVKLMVEMEKKLEKGQSIDDMIPAQK |
| 75 | Truncation of | GGGGGGEYMEQEEDWDRDLLLDPAWEKQQRKTFTAWCNSHLRKAGTQIEN |
| | Rabbit | IEEDFRNGLKLMLLLEVISGERLPRPDKGKMRFHKIANVNKALDFIASKGVKL |
| | G1U4H8 | VSIGAEEIVDGNLKMTLGMIWTIILRFAIQDISVEETSAKEGLLLWCQRKTAPY |
| | (Actinin Alpha | RNVNVQNFHTSWKDGLALCALIHRHRPDLIDYAKLRKDDPIGNLNTAFEVAE |
| | 3 | KYLDIPKMLDAEDIVNTPKPDEKAIMTYVSCFYHAFAGAEQAETAANRICKV |
| | | LAVNQENEKLMEEYEKLASELLEWIRRTVPWLENRVGEPSMSAMQRKLEDF |
| | | RDYRRLHKPPRVQEKCQLEINFNTLQTKLRLSHRPAFMPSEGKLVSDIANAW |
| | | RGLEQVEKGYEDWLLSEIRRLQRLQHLAEKFQQKASLHETWTRGKEDMLTQ |
| | | RDYESASLQEVRALLRRHEAFESDLAAHQDRVEHIAALAQELNELDYHEAAS |
| | | VNSRCQAICDQWDNLGTLTQKRRDALERMEKLLETIDQLQLEFARRAAPENN |
| | | WLDGAIEDLQDVWLVHSVEETQSLLTAHDQFKATLPEADRERGAILGIQGEIQ |
| | | KICQTYGLRPSSTNPYINLSPQDINTKWDTVRKLVPSRDQTLQEELARQQMNE |
| | | RLRRQFAAQANAVGPWIQAKVEEVGRLAAGLAGSLEEQMAGLRQQEQNIIN |
| | | YKSNIDRLEGDHQLLQESLVFDNKHTVYSMEHIRVGWEQLLTSIARTINEVEN |
| | | QVLTRDAKGLSQEQLNEFRASFNHFDRKRNGMMEPDDFRACLISMYDLGEV |
| | | EFARIMTMVDPNAAGVVTFQAFIDFMTR |
| 76 | Truncation of | IYYLSLEFYMGRTLQNTMVNLALENACDEATYQLGLDMEELEEIEEDAGLGN |
| | Rabbit PYGM | GGLGRLAACFLDSMATLGLAAYGYGIRYEFGIFNQKICGGWQMEEADDWLR |
| | (Glycogen | YGNPWEKARPEFTLPVHFYGRVEHTSQGAKWVDTQVVLAMPYDTPVPGYR |
| | Phosphorylase, | NNVVNTMRLWSAKAPNDFNLKDFNVGGYIQAVLDRNLAENISRVLYPNDNF |
| | Muscle Form) | FEGKELRLKQEYFVVAATLQDIIRRFKSSKFGCRDPVRTNFDAFPDKVAIQLN |
| | | DTHPSLAIPELMRVLVDLERLDWDKAWEVTVKTCAYTNHTVLPEALERWPV |
| | | HLLETLLPRHLQIIYEINQRFLNRVAAAFPGDVDRLRRMSLVEEGAVKRINMA |
| | | HLCIAGSHAVNGVARIHSEILKKTIFKDFYELEPHKFQNKTNGITPRRWLVLCN |
| | | PGLAEIIAERIGEEYISDLDQLRKLLSYVDDEAFIRDVAKVKQENKLKFAAYLE |
| | | REYKVHINPNSLFDVQVKRIHEYKRQLLNCLHVITLYNRIKKEPNKFVVPRTV |
| | | MIGGKAAPGYHMAKMIIKLITAIGDVVNHDPVVGDRLRVIFLENYRVSLAEK |
| | | VIPAADLSEQISTAGTEASGTGNMKFMLNGALTIGTMDGANVEMAEEAGEEN |
| | | FFIFGMRVEDVDRLDQRGYNAQEYYDRIPELRQIIEQLSSGFFSPKQPDLFKDI |
| | | VNMLMHHDRFKVFADYEEYVKCQERVSALYKNPREWTRMVIRNIATSGKFS |
| | | SDRTIAQYAREIWGVEPSRQRLPAPDEKIP |
| 77 | Truncation of | SHPALTPEQKKELSDIAHRIVAPGKGILAADESTGSIAKRLQSIGTENTEENRRF |
| | Rabbit ALDOA | YRQLLLTADDRVNPCIGGVILFHETLYQKADDGRPFPQVIKSKGGVVGIKVDK |
| | (Fructoase- | GVVPLAGTNGETTTQGLDGLSERCAQYKKDGADFAKWRCVLKIGEHTPSAL |
| | Bisphosphate | AIMENANVLARYASICQQNGIVPIVEPEILPDGDHDLKRCQYVTEKVLAAVYK |
| | Aldolase A) | ALSDHHIYLEGTLLKPNMVTPGHACTQKYSHEEIAMATVTALRRTVPPAVTG |
| | | VTFLSGGQSEEEASINLNAINKCPLLKPWALTFSYGRALQASALKAWGGKKE |
| | | NLKAAQEEYVKRALANSLACQGKYTPSGQAGAAAS |
| 78 | Rabbit ALBU | EAHKSEIAHRENDVGEEHFIGLVLITFSQYLQKCPYEEHAKLVKEVTDLAKAC |
| | (Albumin) | VADESAANCDKSLHDIFGDKICALPSLRDTYGDVADCCEKKEPERNECFLHH |
| | | KDDKPDLPPFARPEADVLCKAFHDDEKAFFGHYLYEVARRHPYFYAPELLYY |
| | | AQKYKAILTECCEAADKGACLTPKLDALEGKSLISAAQERLRCASIQKFGDRA |
| | | YKAWALVRLSQRFPKADFTDISKIVTDLTKVHKECCHGDLLECADDRADLAK |
| | | YMCEHQETISSHLKECCDKPILEKAHCIYGLHNDETPAGLPAVAEEFVEDKDV |
| | | CKNYEEAKDLFLGKFLYEYSRRHPDYSVVLLLRLGKAYEATLKKCCATDDP |
| | | HACYAKVLDEFQPLVDEPKNLVKQNCELYEQLGDYNFQNALLVRYTKKVPQ |
| | | VSTPTLVEISRSLGKVGSKCCKHPEAERLPCVEDYLSVVLNRLCVLHEKTPVS |
| | | EKVTKCCSESLVDRRPCFSALGPDETYVPKEFNAETFTFHADICTLPETERKIK |
| | | KQTALVELVKHKPHATNDQLKTVVGEFTALLDKCCSAEDKEACFAVEGPKL |
| | | VESSKATLG |

An animal protein can comprise a cow protein (Table 3). A cow protein can comprise a cow muscle protein. A cow protein can comprise a cow blood protein. A cow protein can comprise a cow albumin protein. A cow protein can comprise a sequence that has at least or about 70%, at least or about 75%, at least or about 80%, at least or about 90%, at least or about 91%, at least or about 92%, at least or about 93%, at least or about 94%, at least or about 95%, at least or about 96%, at least or about 97% at least or about 98% at least or about 99% or about 100% sequence identity to any one of SEQ ID NO: 79-93.

A food product may comprise a fragment or a truncation of a cow protein. The food product may include a fragment or truncation of any one of SEQ ID Nos: 79-93. A sequence of the fragment or truncation may be identical or substantially identical to a portion of any one of SEQ ID Nos: 79-93. A sequence of the fragment or truncation may be at least about 70%, 80%, 90%, 95%, 98%, or more identical to a portion of a sequence of any one of SEQ ID Nos: 79-93. A length of the fragment or truncation may be at least about 10%, 15%, 20%, 30%, 40%, 50%, 60%, 70%, 80%, 90%, or more of a length of any one of SEQ ID Nos: 79-93. A length of the fragment or truncation may be less than or equal to about 90%, 80%, 70%, 60%, 50%, 40%, 30%, 20%, 15%, 10%, or less than a length of any one of SEQ ID Nos: 79-93.

TABLE 3
Cow Proteins

| SEQ | | |
|---|---|---|
| ID NO | Description | Amino Acid Sequence |
| 79 | Cow KCRM | MPFGNTHNKHKLNFKAEEEYPDLSKHNNHMAKALTLEIYKKLRDKETPSG |
| | (Creatine kinase | FTLDDVIQTGVDNPGHPFIMTVGCVAGDEESYTVFKDLFDPIIQDRHGGFKP |
| | M-type) | TDKHKTDLNHENLKGGDDLDPNYVLSSRVRTGRSIKGYALPPHCSRGERR |
| | | AVEKLSVEALNSLTGEFKGKYYPLKSMTEQEQQQLIDDHFLFDKPVSPLLL |
| | | ASGMARDWPDARGIWHNDNKSFLVWVNEEDHLRVISMEKGGNMKEVFR |
| | | RFCVGLQKIEEIFKKAGHPFMWNEHLGYVLTCPSNLGTGLRGGVHVKLAH |
| | | LSKHPKFEEILTRLRLQKRGTGGVDTAAVGSVFDVSNADRLGSSEVEQVQL |
| | | VVDGVKLMVEMEKKLEKGQSIDDMIPAQK |
| 80 | Cow ACTN3 | MMMVLQPEGLGTGEGPFAGGRGGGEYMEQEEDWDRDLLLDPAWEKQQR |
| | (Alpha-actinin-3) | KTFTAWCNSHLRKAGTQIENIEEDFRNGLKLMLLLEVISGERLPRPDKGKM |
| | | RFHKIANVNKALDFIASKGVKLVSIGAEEIVDGNLKMTLGMIWTIILRFAIQ |
| | | DISVEETSAKEGLLLWCQRKTAPYRNVNVQNFHTSWKDGLALCALIHRHR |
| | | PDLIDYAKLRKDDPIGNLNTAFEVAEKYLDIPKMLDAEDIVNTPKPDEKAIM |
| | | TYVSCFYHAFAGAEQAETAANRICKVLAVNQENEKLMEEYEKLASELLEW |
| | | IRRTVPWLENRVGEPSMSAMQRKLEDFRDYRRLHKPPRVQEKCQLEINFNT |
| | | LQTKLRLSHRPAFMPSEGKLVSDIANAWRGLEQAEKGYEDWLLSEIRRLQR |
| | | LQHLAEKFQQKASLHEAWTRGKEDMLSQRDYETASLQEVRALLRRHEAFE |
| | | SDLAAHQDRVEHIAALAQELNELDYHEAASVNSRCQAICDQWDNLGTLTQ |
| | | KRRDALERMEKLLETIDQLQLEFARRAAPFNNWLDGAVEDLQDVWLVHS |
| | | VEETQSLVTAHDQFKATLPEADRERGAILGIQGEIQKICQTYGLRPSSTNPYI |
| | | TLTPQDINTKWDTVRKLVPSRDQMLQEELTRQQVNERLRRQFAAQANAIG |
| | | PWIQGKVEEVGRLAAGMAGSLEEQMAGLRQQEQNIINYKSNIDRLEGDHQ |
| | | LLQESLVFDNKHTVYSMEHIRVGWEQLLTSIARTINEVENQVLTRDAKGLS |
| | | QEQLNEFRASFNHFDRKRNGMMEPDDFRACLISMGYDLGEVEFARIMTMV |
| | | DPNAAGVVTFQAFIDFMTRETAETDTAEQVVASFKILAGDKNYITAEELRR |
| | | ELPAEQAEYCIRRMAPYKGAGAPAGALDYVAFSSALYGESDL |
| 81 | Cow PYGM | MSRPLTDQEKRKQISVRGLAGVENVTELKKNFNRHLHFTLVKDRNVATPR |
| | (Glycogen | DYYFALAYTVRDHLVGRWIRTQQHYYEKDPKRIYYLSLEFYIGRTLQNTM |
| | phosphorylase, | VNLALENACDEATYQLGLDMEELEEIEEDAGLGNGGLGRLAACFLDSMAT |
| | muscle form) | LGLAAYGYGIRYEFGIFNQKISGGWQMEEADDWLRYGNPWEKARPEFTLP |
| | | VHFYGRVEHTSQGAKWVDTQVVLAMPYDTPVPGYRNNVVNTMRLWSAK |
| | | APNDFNLKDFNVGGYIQAVLDRNLAENISRVLYPNDNFFEGKELRLKQEYF |
| | | VVAATLQDIIRRFKSSKFGCLDPVRTNFDAFPDKVAIQLNDTHPSLAIPELM |
| | | RILVDQERLEWEKAWEVTVKTCAYTNHTVLPEALERWPVHLIETLLPRHLQ |
| | | IIYEINQRFLNRVAAAFPGDVDRLRRMSLVEEGAVKRINMAHLCIAGSHAV |
| | | NGVARIHSEILKKTIFKDFYELEPHKFQNKTNGITPRRWLVMCNPGLAEIIAE |
| | | RIGEEYIADLDQLRKLLSYVDDESFIRDVAKVKQENKLKFSAYLEKEYKVHI |
| | | NPNSLFDIQVKRIHEYKRQLLNCLHVITLYNRIKKEPNKFFVPRTVMIGGKA |
| | | APGYHMAKMIIKLITAIGDVVNHDPVVGDRLRVIFLENYRVSLAEKVIPAA |
| | | DLSEQISTAGTEASGTGNMKFMLNGALTIGTMDGANVEMAEEAGEENFFIF |
| | | GMRVEDVERLDQKGYNAQEYYDRIPELRHVIDQLSSGFFSPKQPDLFKDIV |
| | | NMLMHHDRFKVFADYEEYIKCQERVSALYKNPREWTRMVIRNIATSGKFS |
| | | SDRTIAQYAREIWGVEPTRQRMPAPDEKI |
| 82 | Cow A6QLL8 | MPHQYPALTPEQKKELCDIAHRIVAPGKGILAADESTGSIAKRLQSIGTENTE |
| | (Fructose- | ENRRFYRQLLLTADDRVNPCIGGVILFHETLYQKADDGRPFPQVIKAKGGV |
| | bisphosphate | VGIKVDKGVVPLAGTNGETTTQGLDGLSERCAQYKKDGADFAKWRCVLKI |
| | aldolase) | GEHTPSSLAIMENANVLARYASICQQNGIVPIVEPEILPDGDHDLKRCQYVT |
| | | EKVLAAVYKALSDHHIYLEGTLLKPNMVTPGHACTQKYSHEEIAMATVTA |
| | | LRRTVPPAVPGITFLSGGQSEEEASINLNAINKCPLLKPWALTFSYGRALQAS |
| | | ALKAWGGKKENLKAAQEEYVKRALANSLACQGKYTPSGKAGAAASESLFI |
| | | SNHAY |
| 83 | Cow MYL1 | MAPKKDVKKPAAAAAPAPAPAPAPAPAPAPPKEEKIDLSAIKIEFSKQQQDE |
| | (Myosin light | FKEAFLLFDRTGECKITLSQVGDVLRALGTNPTNAEVKKVLGNPSNEEMNA |
| | chain 1/3, skeletal | KKIEFEQFLPMLQAISNNKDQGTYEDFVEGLRVFDKEGNGTVMGAELRHV |
| | muscle isoform) | LATLGEKMKEEEVEALMAGQEDSNGCINYEAFVKHIMSN |
| 84 | Cow G3P | MVKVGVNGFGRIGRLVTRAAFNSGKVDIVAINDPFIDLHYMVYMFQYDST |
| | (Glyceraldehyde- | HGKFNGTVKAENGKLVINGKAITIFQERDPANIKWGDAGAEYVVESTGVFT |
| | 3-phosphate | TMEKAGAHLKGGAKRVIISAPSADAPMFVMGVNHEKYNNTLKIVSNASCT |
| | dehydrogenase) | TNCLAPLAKVIHDHFGIVEGLMTTVHAITATQKTVDGPSGKLWRDGRGAA |
| | | QNIIPASTGAAKAVGKVIPELNGKLTGMAFRVPTPNVSVVDLTCRLEKPAK |
| | | YDEIKKVVKQASEGPLKGILGYTEDQVVSCDFNSDTHSSTFDAGAGIALND |
| | | HFVKLISWYDNEFGYSNRVVDLMVHMASKE |
| 85 | Cow TPM1 | MDAIKKKMQMLKLDKENALDRAEQAEADKKAAEDRSKQLEDELVSLQKK |
| | (Tropomyosin | LKATEDELDKYSEALKDAQEKLELAEKKATDAEADVASLNRRIQLVEEEL |
| | alpha-1 chain) | DRAQERLATALQKLEEAEKAADESERGMKVIESRAQKDEEKMEIQEIQLKE |
| | | AKHIAEDADRKYEEVARKLVIIESDLERAEERAELSEGKCAELEEELKTVTN |
| | | NLKSLEAQAEKYSQKEDKYEEEIKVLSDKLKEAETRAEFAERSVTKLEKSID |
| | | DLEDELYAQKLKYKAISEELDHALNDMTSI |
| 86 | Cow E1BNV1 | MPEAKPAAKKAPAGKDAAAKPAPKEATPQEAPAAPPTEAPPEDQSPTAEEP |
| | (Myosin binding | TGVFLKKPDSVSVETGKDTVIVAKLNGKELPAKPAVKWFKGKWLELGSKS |
| | protein C2) | GARFSFKESHDAASNVYTIELHITKVVLGDRGDYRIEVKAKDFCDSCAFNID |
| | | VEAPRQDSAGQSLESFKRSGEAKSDTAGELDFSGLLKKRQVVEEEKKKKK |
| | | DDDDLGIPPEIWELLKGAKKSEYERIAFQYGITDLRGMLKRLKKAKVEVKK |
| | | SAAFTKKLDPAYQVDRGNKIKLVVEISDPDLPLKWFKNGQEIKPSSKYVFE |
| | | NVGKKRILTINKCTLADDAAYEVVVKDEKCFTELFVKEPPVLIVTPLEDQQ |
| | | VFVGDRVEMAVEVSEEGAQVMWLKDGVELTREDSFKARYRLKKDGKRHI |
| | | LIYSEVTMEDKGHYQVMTNGGQCEAELIVEEKQLEVLQDIADLTVKASEQ |
| | | AVFKCEVSDEKVTGKWYKNGVEVRPSKRITISHTGRFHKLVIDDVRPEDEG |
| | | DYTFVPDGYALSLSAKLKFLEIKVEYVPKQEPPKIHLDCSGQTSENAIVVVA |
| | | GNKLRMDVSITGEPRPVATWMKEDEVFTGTEGRVRIEQRGDISSFVIESAER |
| | | GDEGRYTIKVTNPVGEDVASILLKVVDVPDPPEAVRVTSVGEDWAILVWEP |
| | | PKYDGGRPVTGYLLERKKKGSQRWMKLNFEVFTETTYESTNMIEGILYEM |
| | | RVFAVNVIGVSQPSMNTKPFMPIAPTSEPQHLTVEDVTDTTTTLKWRPPDKI |
| | | GAGGIDGYLIEYCVEGSDEWIPANTEPVERCGFTVKNLPTGAKITFRVVGV |
| | | NIAGRSQPATLAQPVTIREIVEQPKIRLPRHLRQTYVRKVGEHLNLVIPFQGK |
| | | PRPQVVWTKGGAPIDTSHVHVRTSDFDTVFFVRQAARSDSGEYELTVQIEN |
| | | MKDTATVCIRVVEKAGPPQNVMVKEVWGTNALVEWQPPKDDGNSEITGY |
| | | FVQKADKKTMEWFTVYERNRHTSCTVSDLIMGNEYYFRVYSENVCGLSDL |
| | | PGVSKNTARIVKTGMTLKLPEYKEHDFRTPPKFLTPLPDRVVVAGYAAALN |
| | | CAVRGHPKPKVVWMKNKMEIREDPKFLMTNQQGVLTLNIRRPSPFDSGTY |
| | | SCRAVNELGEALAECKLEVRVPQ |
| 87 | Cow E1BF23 | MSLVAVPFYQKRHKHFDQSYRNIQTRYLLDEYSAKKKRASAQSSLQRSVT |
| | (Myomesin 2) | RKSSSQRESSRAALGESTLGETTLGETSLGDTTLGRTTCRLCAKRMSSALEE |
| | | EAQERKRRYQSMVAAYGEAKRERYLSELAQLEEDVHVARTHARDQLDRL |
| | | ALQHAGRHGLHHAVDDRLAWERHSFEERISRAPEILVRLRSHTVWERMSV |
| | | KLCFTVQGFPTPVVQWYKDGSLICQAGEPGKYRIDSKYGVHTLEINRADFD |
| | | DTATYSAVATNVHGQVSTNAAVVVRRFRGDEEPFHSVGLPIGLPLSSVIPYT |
| | | HFDVQFIEKFGVTFRREGETLTLKCTLLVTPDLKRVQPRAEWYRDDVLLKE |
| | | SKWTKMFFGEGQASLSFSHLNKDDEGLYTLRIVSRGGITDHSAFLFVRDAD |
| | | PLVTGAPGAPMDVHCHDANRDYVIVTWKPPNATKESPVIGYFIDRCEVGTD |
| | | NWIQCNDAPVKICKYPVTGLFEGRSYIFRVRAVNSAGISRPSRVSEAVAALD |
| | | PVDLRRLQAVHLEGEKDIVVYQEELEGEVQIPGPPTNVHASETSRTYVVLS |
| | | WDPPEPRGKEPLMYFIEKSMVGSGSWQRVNAQTAVRSPRYAVFDLAEGKP |
| | | YVFRVLSANKHGLSDPSEITAPIQAQDTIVVPSAPGRVLASRNTRTSVVVQW |
| | | DRPKHEEDLLGYYVDCSVAGSNVWEPCNHKPIGYNRFVVHGLTTGEQYIF |
| | | RVKAVNAVGTSENSQESDVIKVQAALTVPSHPYGITLLNCDGHSMILGWK |
| | | VPKFSGGSPILGYYVDKREAHHKNWHEVNSSPLKERILTVEGLTEGSLYEF |
| | | KIAAANMAGIGQPSDPSELFKCEAWTMPEPGPAYDLTFCEVRDTSLVLLWK |
| | | PPVYSGSSPVSGYFVDYKEEDSGEWLTVHETATPHRYLKVCDLHQGKTYV |
| | | FRVRAVNASGVGRPSDTSEPVLVEARPGTKEVSAGVDEEGNVYLSFDCPEV |
| | | TDASHFTWCKSYEDIADDDRFKVETAGDHSKLYFKNLDKEDLGTYSVSVS |
| | | DTDGVSSSFVLDEEELERLKALSNEIKNPTIPLKSELAYEIFDKGQVRFWLQ |
| | | AEHLSPDSSYRFIINDREVADSETHRIKCDKSTGIIEMVMDRFTIDNEGTYTV |
| | | QIQDGKAKSQSSLVLIGDAFKAVLKEAEFQRKEFLRKQGPHFAEYLHWDVT |
| | | EECEVRLVCKVANTKKETVFKWLKDDVLYETEKMPDLEKGVCELLIPKLS |
| | | KKDHGEYKATLKDDRGQDVSTLEIAGKVYEDMILAMSRVCGASASPLKIL |
| | | CTPEGIRLQCFMKYFTEEMKVNWYHKDAKITSSEHMRIGGNEQMAWLQIC |
| | | EPTEKDKGKYTFEILDGKKNHQRSLDLSGQAFDEAFAEFQQLKAAAFAEKN |
| | | RGKVIGGLPDVVTIMEGKTLNLTCTVFGNPDPEVVWFKNDKDIELSDHFSV |
| | | KVEQGKYVSMTIKGVTSEDSGKYSIHVKNKYGGEKIDVTVSVYKHGEEIPD |
| | | VVLPQQAKPKLLPAAAPSPAH |
| 88 | Cow ENOB | MAMQKIFAREILDSRGNPTVEVDLHTAKGRFRAAVPSGASTGIYEALELRD |
| | (Beta-enolase) | GDKSRYLGKGVLKAVEHINKTLGPALLEKKLSVVDQEKVDKFMIELDGTE |
| | | NKSKFGANAILGVSLAVCKAGAAEKGVPLYRHIADLAGNPELILPVPAFNVI |
| | | NGGSHAGNKLAMQEFMILPVGASSFREAMRIGAEVYHHLKGVIKAKYGKD |
| | | ATNVGDEGGFAPNILENNEALELLKTAIQAAGYPDKVVIGMDVAASEFYRN |
| | | GKYDLDFKSPDDPARHISGEKLGELYKNFIKNYPVVSIEDPFDQDDWATWT |
| | | SFLSGVNIQIVGDDLTVTNPKRIAQAVEKKACNCLLLKVNQIGSVTESIQAC |
| | | KLAQSNGWGVMVSHRSGETEDTFIADLVVGLCTGQIKTGAPCRSERLAKY |
| | | NQLMRIEEALGDKAVFAGRKFRNPKAK |
| 89 | Cow MLRS | MAPKKAKRRAAAEGGSSSVFSMFDQTQIQEFKEAFTVIDQNRDGIIDKEDL |
| | (Myosin | RDTFAAMGRLNVKNEELDAMMKEASGPINFTVFLNMFGEKLKGADPEDVI |
| | regulatory light | TGAFKVLDPEGKGTIKKKFLEELLTTQCDRFSQEEIKNMWAAFPPDVGGNV |
| | chain 2) | DYKNICYVITHGDAKDQE |
| 90 | Cow MYH1 | MSSDQEMAVFGEAAPYLRKSEKERIEAQNKPFDAKTSVFVADPKESFVKAT |
| | (Myosin-1) | VQSREGGKVTAKTEAGATVTVKEDQVFPMNPPKFDKIEDMAMMTHLHEP |
| | | AVLYNLKERYAAWMIYTYSGLFCVTVNPYKWLPVYNAEVVTAYRGKKRQ |
| | | EAPPHIFSISDNAYQFMLTDRENQSILITGESGAGKTVNTKRVIQYFATIAVT |
| | | GEKKKEEPTSGKMQGTLEDQIISANPLLEAFGNAKTVRNDNSSRFGKFIRIH |
| | | FGTTGKLASADIETYLLEKSRVTFQLKAERSYHIFYQIMSNKKPELIEMLLIT |
| | | TNPYDYAYVSQGEITVPSIDDQEELMATDSAIEILGFTSDERVSIYKLTGAV |
| | | MHYGNLKFKQKQREEQAEPDGTEVADKAAYLQGLNSADLLKALCYPRVK |
| | | VGNEFVTKGQTVEQVYNAVGALAKAVYDKMFLWMVARINQQLDTKQPR |
| | | QYFIGVLDIAGFEIFDFNSLEQLCINFTNEKLQQFFNHHMFVLEQEEYKKEGI |
| | | EWEFIDFGMDLAACIELIEKPMGIFSILEEECMFPKATDMSFKNKLYEQHLG |
| | | KSNNFQKPKPAKGKAEAHFSLIHYAGTVDYNITGWLDKNKDPLNETVVGL |
| | | YQKSSVKTLALLFSGPASGEAEGGPKKGGKKKGSSFQTVSALFRENLNKLM |
| | | TNLRSTHPHFVRCIIPNETKTPGAMEHELVLHQLRCNGVLEGIRICRKGFPSR |
| | | ILYADFKQRYKVLNASAIPEGQFIDSKKASEKLLASIDVDHTQYKFGHTKVF |
| | | FKAGLLGLLEEMRDEKLAQLITRTQARCRGFLARVEYQKMVERRESIFCIQ |
| | | YNVRAFMNVKHWPWMKLYFKIKPLLKSAETEKEMANMKEEFEKTKEELA |
| | | KSEAKRKELEEKMVTLTQEKNDLQLQVQSEADALADAEERCDQLIKTKIQL |
| | | EAKIKEVTERAEDEEEINAELTAKKRKLEDECSELKKDIDDLELTLAKVEKE |
| | | KHATENKVKNLTEEMAGLDETIAKLTKEKKALQEAHQQTLDDLQAEEDK |
| | | VNTLTKAKTKLEQQVDDLEGSLEQEKKLRMDLERAKRKLEGDLKLAQEST |
| | | MDIENDKQQLDEKLKKKEFEMSNLQSKIEDEQALAMQLQKKIKELQARIEE |
| | | LEEEIEAERASRAKAEKQRSDLSRELEEISERLEEAGGATSAQIEMNKKREA |
| | | EFQKMRRDLEEATLQHEATAAALRKKHADSVAELGEQIDNLQRVKQKLEK |
| | | EKSEMKMEIDDLASNMETVSKAKGNLEKMCRALEDQLSELKTKEDEQQRL |
| | | INDLTTQRARLQTESGEFSRQLDEKDALVSQLSRGKQAFTQQIEELKRQLEE |
| | | EIKAKSALAHALQSARHDCDLLREQYEEEQEGKAELQRAMSKANSEVAQ |
| | | WRTKYETDAIQRTEELEEAKKKLAQRLQDAEEHVEAVNAKCASLEKTKQR |
| | | LQNEVEDLMIDVERTNAACAALDKKQRNFDKILSEWKQKYEETHAELEAS |
| | | QKESRSLSTELFKIKNAYEESLDQLETLKRENKNLQQEISDLTEQIAEGGKRI |
| | | HELEKVKKQVEQEKSEIQAALEEAEASLEHEEGKILRIQLELNQVKSEIDRKI |
| | | AEKDEEIDQLKRNHIRIVESMQSTLDAEIRSRNDAIRLKKKMEGDLNEMEIQ |
| | | LNHANRMAAEALKNYRSTQAILKDTQIHLDDALRGQEDLKEQLAMVERR |
| | | ANLLQAEIEELRATLEQTERSRKIAEQELLDASERVQLLHTQNTSLINTKKK |
| | | LETDITQIQGEMEDIIQEARNAEEKAKKAITDAAMMAEELKKEQDTSAHLE |
| | | RMKKNLEQTVKDLQHRLDEAEQLALKGGKKQIQKLEARVRELEGEVESEQ |
| | | KRNVEAVKGLRKHERRVKELTYQTEEDRKNILRLQDLVDKLQAKVKSYKR |
| | | QAEEAEEQSNVNLSKFRKLQHELEEAEERADIAESQVNKLRVKSREVHTKII |
| | | SEE |
| 91 | Cow | MPNFQTRTRFYETTTTRTSDYEQSETSRPALAQPVPEKPVERKRVIRKKVDP |
| | A0A3Q1M707 | SKFMTPYIAHSQKMQNLFSTNKYKENYEKAKGKPYAITTDTPELRRIKKVQ |
| | (Nebulin) | DQLSEVKYRVDGDVAKTICHVDEKAKDIEHAKKVSQQVSKVLYKQNWED |
| | | TKDKYLLPPDAPELVQAIKNTAMFSKKLYTEDWEADKTMFYPYNDSPELR |
| | | RVAQAQKALSDIAYKKGLAEQQTQFTSLPDPPDIEFAKKVTNQVSKQKYKE |
| | | DYENKVKGKWSETPCFEIATARMNSNNISTKKYQEDFEHMKDQIYFMQTE |
| | | TPEYKVNKQAGVAASKVKYKQDYEKNKGKADYNVLPASENPLLRQLKAA |
| | | GDALSDKLYKENYEKTKAKSINYCETPKFKLDTVLHNFSSDTKYKDSYLKD |
| | | ILGHYVGSYEDPYHTHCMRVSAQNSDKNYKAEYEEDRGKGFFPQTITQEY |
| | | EAIKKLDQCKDHTYKVHPDKTKFTQVTDSPVLMQAQVNSKQLSDLNYKA |
| | | KHENEKFKCHIPPDTPAFIQHKLNAYNLSDNIYKHDWEKTKAKKFDIKVDA |
| | | IPLLAAKANTKIASDVMYKKDYEKSKGKMIGALSINDDPKMLHSLKTAKN |
| | | QSDRLYKENYEKTKAKSMNYCETPKYQLDTLLKNFSEAKYKDSYVQNVL |
| | | GHYIGSFEDPYQVHCLKISAQNSDKNYKAEYEEDKGKCYFPQTITQEYEAIK |
| | | KLDQCKDHTYKVHPDKTKFTAVTDTPVLLQAQLNTKQLSDLNYKAKHEG |
| | | EKFKCHIPADAPQFIQHRVNAYNLSDNIYKHDWEKTKAKKFDIKVDAIPLL |
| | | AAKANTKIASDVMYKKDYEKSKGKMIGALSINDDPKMLHSLKTAKNQSD |
| | | HEYRKDYEKSKTIYTAPLDMLPLTHAKKSQAIASDVDYKHLLHNYSYPPDS |
| | | VNVDLAKKAYALQSDVEYKADYNSWMKGCGWMPFGSLEMEKAKRASDI |
| | | LNEKKYRQHPDTLKFTSIEDAPIIVQSKINQAQRSDVAYKAKGEEVIHKYSL |
| | | PADLPQFIQAKVNAYNISENLYKADLKDLSKKGYDLRIDAIPIKAAKAARQ |
| | | AASDVQYKKDYEKAKGKMVGFQSLQDDPKLVHYMNVAKIQSDREYKKA |
| | | YEKTKTRHNTPHDMVNIVAAKKAQDVASNVNYKHSLHHYTYLPDAMDLE |
| | | LSKNMMHIQSDNVYKEDYNNWMKGIGWIPIGSLEVEKVKKAGDALNEKK |
| | | YRQHPDTLKFTSIVDSPVMVQAKQNTQQVSDILYKAKGEDVKHKYTMSPD |
| | | LPQFLQAKCNAYNLSDVCYKRDWHDLIAKGTNVLGDAIPITAAKASRNIAS |
| | | DYKYKEAYEKAKGKQVGFRSLQDDPKLVHYMNVAKLQSDREYKKNYEN |
| | | TKTSYHTPGDMVSITAAKMAQDVATNVNYKQPIHHYTYLPDALSLEHIRN |
| | | VNQIQSDNVYKDEYNHFFKGMGWIPIGSLEVEKVKKAGDALNEKKYRQHP |
| | | DTIKFTSVPDSMGMVLAQHNTKQLSDLNYKVEGEKVKHKYTMDPDVPQFI |
| | | QAKVNAYNMSDSHYKADWKKTLAKGYDLRPDAIPIVAAKSSRNIASDFKY |
| | | KEAYEKTKGKQIGFRSLQDDPKLVHFMNVAKMQSDREYKKGYEASKTKY |
| | | HTPLDMLSVTAAKKSQEVATNTNYKQPFHHYTLLPDALNVEHSRNAMQIQ |
| | | SDNLYKSDFTNWMKGIGWLPLESLEVEKAKKAGEILSEKKYRQHPEKLKFT |
| | | YAMDTMEQALNKSNKLIMDKRLYTEKWNKDKTTIHVMPDTPDILLSRVNQ |
| | | ITMSDKLYKAGWEEEKKKGYDLRPDAISIKAARASRDIASDYKYKQAYEQ |
| | | AKGKQIGFRSLEDDPKLVHFMQVAKMQSDREYKKAYEKSKTSFQTPVDML |
| | | SVVAAKKSQEVATNANYRNVIHTYNMLPDAMSLELAKNMMQIQSDNQYK |
| | | ADYADFMKGIGWLPLGSLEAEKNKKAMEIISEKKYRQHPDTLKYSTLMDS |
| | | MNMVLAKNNAKIMNEHLYKQAWEADKTKVHIMPDIPQIILAKANAINISD |
| | | KLYKLSLEEAKKKGYDLRTDAIPIKAAKASRDIASDYKYKHSYEKERGKM |
| | | VGFRSLEDDPKLVHSMQVAKMQSDREYKKNYEKTKTSYHTPADMLSVTA |
| | | AKDAQANITNTNYKHLIHKYILLPDAMNIQLSKNMNRIQSDNEYKQDYNE |
| | | WYKGLGWSPAGSLEVEKAKKATEYASDQKYRQHPSNFQFTKLNDSMDMV |
| | | LAKQNAHTMNKYLYTVDWNKDKTKIHVMPDTPDILQAKQNQTMYSQKL |
| | | YKLGWEEALKKGYDLPVDAISVQAAKASRDIVSDFKYKQGYRKQIGHHIG |
| | | FRSLQDDPKLVLSMNVAKMQSEREYKKDFEKWKTKFSSPVDMLGVVLAK |
| | | KCQALVSDVDYKNYLHGWTCLPDQNDVIHAKKAYDLQSENLYKSDLEWL |
| | | KGIGWSPLGSLEAEKNKRASEIISEKKYRQPPDRNKFTSIPDAMDIVLAKTN |
| | | AKNRSDILYREAWDKDKTQIHIMPDTPDIILAKANLINTSDKFYRMGYEELR |
| | | KKGYDLPVDAIPIKAAKASREIASEYKYKEGFRMQLGHHIGARNIKDDPKM |
| | | MWSMHVAKIQSDREYKKDFEKWKTKFSSPVDMLGVVLAKKCQTLVSDID |
| | | YKNYLHQWTCLPDQSDVIHARRAYDLQSDNLYKSDLQWLRGIGWLPSGSL |
| | | EDEKNKRASQILSDHVYRQHPDQFKFSSLMDSIPMVLAKNNAITMNHRLYT |
| | | EAWDKDKTTVHIMPDTPEVLLAKQNQINYSEKLYKLGLDEAKRKGYDMRI |
| | | DAIPIRAAKASRDIASEFKYKEGYRRQLGHHIGARAIHDDPKMMWSMHVA |
| | | KIQSDREYKKDFEKWKTKFSSPVDMLGVVLAKKCQTLVSDIDYKNYLHQ |
| | | WTCLPDQSDVIHARQAYDLQSDNVYKSDLQWLRGIGWVPIGSLDVEKSKR |
| | | ASEILSDKLYRQPPDKFKFTSVTDSLEQVLAKNNAINMNKRLYTEAWDKD |
| | | KTQVHIMPDTPEITLARTNKVNYSENLYKLAHEEAKKKGYDLRSDAIPIVA |
| | | AKASRDIISDYKYKDGYRKQLGHHIGARDIKDDPKMMWSMHVAKIQSDRE |
| | | YKKDFEKWKTKFSSPVDMLGVVLAKKCQTLVSDIDYKNYLHQWTCLPDQ |
| | | SDVIHARQAYDLQSDNVYKSDLQWLRGIGWVPIGSVDVVKCKRAAEILSD |
| | | NLYRQPPDTLKFTSVPDSLEQVLAKNNAINMNKRLYTEAWDKDKTQIHIMP |
| | | DTPEIMLARQNKLNYSETLYKLANEEAKKKGYDLRSDAIPIVAAKASRDIIS |
| | | DYKYKDGYRKQLGHHIGARDIKDDPKMMWSMHVAKIQSDREYKKDFEK |
| | | WKTKFSSPVDMLGVVLAKKCQTLVSDIDYKNYLHQWTCLPDQSDVIHARR |
| | | AYDLQSDNIYKSDLQWLRGIGWVPIGSVDVVKCKRAAEILSDNLYRQRPDT |
| | | LKFTSVPDSLEQVLAKNNAINMNKRLYTEAWDKDKTQIHMMPDTPEITLA |
| | | RQNKINYSENLYRQAMEEAKKEGYDLRSDAIPIVAAKASREIASDYKYKEA |
| | | YRKQLGHHIGARAIHDDPKMMWSVHVAKMQSDREYKKDFEKYKTRFSSP |
| | | VDMLGIVLAKKCQTLVSDVDYKHPLHEWTCLPDQNDIIHARKAYDLQSDN |
| | | LYKSDLEWLKGIGWVPIGSVEVLKAKRAGEILSDNIYRQRPDTLKFTSVTDS |
| | | PEQVLAKNNAINMNKRLYTEAWDNDKKTIHVMPDTPEIMLAKLNRINYSD |
| | | KLYKLALEESRKEGYDLRLDAIPIQAAKASREIASDYKYKEGYRKQLGHHI |
| | | GARNIKDDPKMMWSIHAGKLQSDLEYKKDFEKWKTKFSSPVDMMGLVQA |
| | | KKCQILVSDIDYKNLLHEWTCLPDQNDIIQARKAYDLQSDAIYKADLEWLR |
| | | GIGWVPIDSVGVEHAKRAGEILSERKYRQPAVQLKFTSITDTPEIVLAKNNA |
| | | LNVSKHLYTEAWDADKTSIHVMPDTPEILLAKSNSANISHKLYTKGWDESK |
| | | MKDYDLRADAISIKSAKASRDIASDYKYKEAYEKHKGHHIGARSVEDDPRI |
| | | MCAMNAGRIQSEREYKKEFQKWKTKFSSPVDMLGILLAKKCQTLVSDIDY |
| | | RNYLHHWTCLPDQNDIIQARKAYDLQSDNVYKSDLEWLRGIGWMPEGSVE |
| | | MNRVKNAQDLINERRYRTRPEALTFTSIVDTPEVVLAKANSLQMSEKLYQE |
| | | AWNKDKTNISIPSDTPLILQAQINAMQISNKLYQKDWDEAKQKGYDLRAD |
| | | AIEIKHAKASREIASEYKYKEGYRKQLGHHVGFRTLQDDPKLVWSIHAAKI |
| | | QSDREYRKAYEKSKGIHNTPLDMMSIVQAKKCQVLVSDIDYRNYLHQWIC |
| | | LPDQNDVIQAKKAYELQSDAIYKADLEWLRGIGWMPEGSPEVLRVKNAQH |
| | | IFRDSVYRTPVVKLKYTSIVDTPEVVLAKSNAENISIPKYREVWDKDKTSIHI |
| | | MPDTPEINLARTNALNVSNKLYREGWDEMKMSCDVRLDAIPIQAAKASREI |
| | | ASDYKYKLDHEKQKGHYVGTRTARDDNKIRWALIAGKIQNEREYRLHWA |
| | | KWKSKFQSPPDMLSIEHSKNSQTLVSDIDYRHYLHQWTCMPDQNDVIQAR |
| | | KAYDLQSDAIYKADLEWLRGIGWMPADSVSVNHAKHMADIFNEKKYRTKI |
| | | ETLSFTPVDDRVDYVTAKHSGEILNDIKYRKDWNDTKSKYTLTETPQLHTA |
| | | QEAARILDQYLYKESWEKQKATGYILPPDAVPFVHAHHSGDVQSELKYKA |
| | | EHVKQKGHYVGVPTMRDDPKLVWFEHAGQIQNDRLYKENYHKTKAKIHI |
| | | PPDMVSVLAAKEGQALASDIDYRNYLHQWICHPDQNDVIQARKAYDLQSD |
| | | NIYKADLEWLRGIGWIPLDSVDHVRVTRNQEMMNQIKYKKDALANYPNFT |
| | | SVVDPPEIVLAKINSVNQSDVKYKETFNKLIKGKYIFSPDTPYITHSKDMEKL |
| | | YSTILYKRAWDGTKAYGYTLDERYIPIVGAKHADFVNSELKYKETYEKLK |
| | | GHYLAGKEISEFPNVVHCLDFQKMRSLLNYRRHYEDTKANVHIPQDMMNH |
| | | VLAKRCQYILSDLEYRHYFHQWTSLPEEPNVVRVRHAQEILSDNVYKDDL |
| | | NWLKGIGCYVWDTPQILHAKKSYDLQSQILYTAAGKENLKNYNLVTDTPL |
| | | YVTALQSGINASEVKYKENYHQTKDKYTTVLETVDYDRIKNLKDLFSSNLY |
| | | KEAWDKVKATSYILPPNTVSLTHAKNQKYMASHIKYREEYEKFKALYTLP |
| | | RSVEDDPNTARCLRVGKFNIDRLYRSVYEKNKMKIHIVPDMVEMVTAKDS |
| | | QKKVSEIDYRLHLHEWICHPDLQVNSHVRKVTDQISDIVYKDDLTWLKGIG |
| | | CYVWDTPEILHAKHAYDLRNDIKYKAHVLKTRNNYKLVTDTPVYVQAVK |
| | | SGKQLSDAVYHYDYVHSIRGRVAPTTKTVDLDRALHAYKLQSENLYRKAG |
| | | LHALPTGYRLPVDTPHFKHTKDTRYMSSYFKYKEVYEHMKAYGYTLGPN |
| | | DVPFVNVRRVNNITSERLYRQLYHKLKDKIHTTPDTPEIRQVKKTQEAVSEL |
| | | IYKSDFFKMQGHMISLPYTPQVLHCRYVGDITSDIKYKEDLQVLRGMGCFL |
| | | YDTPDMVRSRHLRKLWSHYLYTDKARKMRDKYKVVLDTPEYRKVQELKT |
| | | HLSELVYRAAGRKQKSIFTSVPDTPDLTRAKRGQKLQSQYLYVELATKERP |
| | | HHHAGNQTTALKHARHVKDMVSENKYKIQYEKMKDKYTPVPDTPILIRAK |
| | | RAYWNASDLRYKETFQKTKGKYHTVKDALDIVYHRTVTDHISKIKYKENY |
| | | MSQLGIWRSIPDRPEHFHHRAVTDAVSDVKYKQDLTWLKGIGCYAYDTPD |
| | | FTLAEKNKTLYSKYKYKEVFERTKSNFKYVADCPINRHFKFATQLMNEKK |
| | | YRADYEQRKDKYHLVVDEPRHLLAKIAGDQISQIKYRKNYEETKDKFTSIV |
| | | DTPEHLRTTKVNKQISDILYKLEYNKAKPRGYTTIHDTPMLLHVRKVKDEV |
| | | SDLKYKEVYQRTKSNCTIEPDAVHIKAAKDAYKVNTNLDYKKKYEATKAY |
| | | WKWTPDRPDFIQAAKSTLQQSDFEYKLDREYLKGCKLSVTDDKDMVLAL |
| | | KNSIIESDLKYKEKHVKERGSCHAVPDTPQILLAKTVSSLVSENKYKSYVKK |
| | | HLAQGSYTTLPETRDTIHVKEVTKNVSDTNYKKKFVKEKGKSNYSIMLEPP |
| | | DVKHAMDVAKKQSNVAYKKDAKENLHYTTVADRPDIKKATQAAKQASE |
| | | VEYRAKHRKEGSHGLSMLGRPDIEMAKKAAKLSSQVQYRENFNKEKGKTP |
| | | KYNPKDSQLYKVMKDANTLASEVKYKADLKKLHKPVTDMKESLIMNHVL |
| | | NTSHLASSYQYKKNYEKSKGHYHTIPDNLEQLHLKEATELQSIVKYKEKYE |
| | | KERGKPMLDFETPTYITAKESQQMQSGKEYRKDYEESIKGRNLTGLEVTPA |
| | | LLHVKYATKIASEKEYRKDLEESIRGKGLSEMEDTPDMLRAKNATQILNEK |
| | | EYKRDLELEVKGRGLNAMANETPDFLRARNATDIASQIKYKQSAEMEKAN |
| | | FTSVVDTPEIIHAQQVKNLSSQKKYKEDAEKCMSYYETVLDTPEMQRVREN |
| | | QKNFSLLQYQYDLKNSKGKITVVQDTPEILRVKENQKNFSSVLYKEDVSPG |
| | | TAIGKTPEMMRVKQTQDHISSVKYKEVIGQGTPIPDLPEVKRVKQTQKHISS |
| | | VMYKENLGTGIPTPVTPEIERVKRNQENFSSNLSLKHEVTSKSIILTKYNSLK |
| | | LPPHTPLPPVLYKENLGKGTPTPITPEMERVKRNQENFSSVLYKENMGKGTP |
| | | LPVTPEMERIKHNQENISSVLYKENMRKATPTPVTPEMERAKRNQENISSVL |
| | | YSDSFRKQIQGKAAYVLDTPEMRRVRETQRHISTVKYHEDFEKHKGCFTPV |
| | | VTDPITERVKKNTQDFSDICYRGIQRKVVEMEQKRNDQDQETITGLRVWRT |
| | | NPGSVFDYDPAEDNIQSRSLHMINAQAQRRSREQSRSASGLSISGGEEKSEH |
| | | SEAAHLSTYSDGGVFFSAASTGYKHARTTELPQQRSSSVATQQTTVSSIPSH |
| | | PSTAGKIFRAMYDYMAADADEVSFKDGDAIVNVQAIDEGWMYGTVQRTG |
| | | RTGMLPANYVEAI |
| 92 | Cow E1BP87 | MSSDQEMAIFGEAAPYLRKSEKERIEAQNKPFDAKTSVFVVDPKESFVKAT |
| | (Myosin heavy | VQSREGGKVTAKTEAGATVTVKEDQVFPMNPPKYDKIEDMAMMTHLHEP |
| | chain 4) | AVLYNLKERYAAWMIYTYSGLFCVTVNPYKWLPVYNAEVVTAYRGKKRQ |
| | | EAPPHIFSISDNAYQFMLTGELDVSIGESGAGKTVNTKRVIQYFATIAVTGEK |
| | | KKEEPTSGKMQGTLEDQIISANPLLEAFGNAKTVRNDNSSRFGKFIRIHFGTT |
| | | GKLASADIETYLLEKSRVTFQLKAERSYHIFYQIMSNKKPELIEMLLITTNPY |
| | | DYAYVSQGEITVPSIDDQEELIATDSAIDILGFTSDERVSIYKLTGAVMHYGN |
| | | LKFKQKQREEQAEPDGTEVADKAAYLQSLNSADLLKALCYPRVKVGNEFV |
| | | TKGQTVQQVYNAVGALAKAVYEKMFLWMVTRINQQLDTKQPRQYFIGVL |
| | | DIAGFEIFDFNSLEQLCINFTNEKLQQFFNHHMFVLEQEEYKKEGIEWEFIDF |
| | | GMDLAACIELIEKPMGIFSILEEECMFPKATDTSFKNKLYEQHLGKSNNFQK |
| | | PKPAKGKAEAHFSLVHYAGTVDYNIAGWLDKNKDPLNETVVGLYQKSAM |
| | | KTLAFLFAGGPSAEEGGGGKKGGKKKGSSFQTVSALFRENLNKLMTNLRS |
| | | THPHFVRCIIPNETKTPGAMEHELVLHQLRCNGVLEGIRICRKGFPSRILYAD |
| | | FKQRYKVLNASAIPEGQFIDSKKASEKLLGSIDIDHTQYKFGHTKVFFKAGL |
| | | LGILEEMRDEKLAQLITRTQAICRGFLMRVEFRKMMERRESIFCIQYNVRAF |
| | | MNVKHWPWMKLYFKIKPLLKSAETEKEMANMKEEFEKTKEELAKSEAKR |
| | | KELEEKMVTLMQEKNDLQLQVQSEADGLADAEERCDQLIKTKIQLEAKIKE |
| | | LTERAEDEEEINAELTAKKRKLEDECSELKKDIDDLELTLAKVEKEKHATE |
| | | NKVKNLTEEMAGLDENIAKLTKEKKALQEAHQQTLDDLQAEEDKVNTLT |
| | | KSKTKLEQQVEDLEGSLEQEKKLRMDLERAKRKLEGDLKLAQESTMDIEN |
| | | DKQQLDEKLKKKEFEMSNLQSKIEDEQALAMQLQKKIKELQARIEELEEEIE |
| | | AERASRAKAEKLRSDLSRELEEISERLEEAGGATSAQIEMNKKREAEFQKM |
| | | RRDLEEATLQHEATAAALRKKHADSVAELGEQIDNLQRVKQKLEKEKSEM |
| | | KMEIDDLASNMETVSKAKGNLEKMCRTLEDQLSEVKSKEEEHQRLINELSV |
| | | QKAHLHTESGEFSRQLEEKDALVSQLSRGKQAFTQQIEELKRQLEEETKAK |
| | | SALAHALQSARHDCDLLREQYEEEQEGKAELQRALSKANSEVAQWRTKYE |
| | | TDAIQRTEELEEAKKKLAQRLQDAEEHVEVVNAKCASLEKTKQRLQNEVE |
| | | DLMLDVERSNAACAALDKKQRNFDKVLSEWKQKYEETQADLEASQKESR |
| | | SLSTELFKVKNAYEESLDQLETLKRENKNLQQEISDLTEQIAEGGKHIHELE |
| | | KVKKQIEQEKGELQAALEEAEASLEHEEGKILRIQLELNQVKSEIDRKIAEK |
| | | DEEIDQLKRNHLRVVESMQSTLDAEIRSRNDALRIKKKMEGDLNEMEIQLN |
| | | HANRQATEAIKNLRNTQGVLKDTQLHLDDAIRGQDDLKEQLAMVERRAN |
| | | LMQAEIEELRASLEQTERSRRVAEQELLDASERVQLLHTQNTSLINTKKKLE |
| | | TDITQIQGEMEDIVQEARNAEEKAKKAITDAAMMAEELKKEQDTSAHLER |
| | | MKKNMEQTVKDLQHRLDEAEQLALKGGKKQIQKLEARVRELENEVENEQ |
| | | KRNAEAVKGLRKHERRVKELTYQTEEDRKNVLRLQDLVDKLQSKVKAYK |
| | | RQAEEAEEQSNVNLAKFRKLQHELEEAEERADIAESQVNKLRVKSREVHTK |
| | | VISEE |
| 93 | Cow ALBU | MKWVTFISLLLLFSSAYSRGVFRRDTHKSEIAHRFKDLGEEHFKGLVLIAFS |
| | (Albumin) | QYLQQCPFDEHVKLVNELTEFAKTCVADESHAGCEKSLHTLFGDELCKVA |
| | | SLRETYGDMADCCEKQEPERNECFLSHKDDSPDLPKLKPDPNTLCDEFKAD |
| | | EKKFWGKYLYEIARRHPYFYAPELLYYANKYNGVFQECCQAEDKGACLLP |
| | | KIETMREKVLASSARQRLRCASIQKFGERALKAWSVARLSQKFPKAEFVEV |
| | | TKLVTDLTKVHKECCHGDLLECADDRADLAKYICDNQDTISSKLKECCDKP |
| | | LLEKSHCIAEVEKDAIPENLPPLTADFAEDKDVCKNYQEAKDAFLGSFLYE |
| | | YSRRHPEYAVSVLLRLAKEYEATLEECCAKDDPHACYSTVFDKLKHLVDE |
| | | PQNLIKQNCDQFEKLGEYGFQNALIVRYTRKVPQVSTPTLVEVSRSLGKVG |
| | | TRCCTKPESERMPCTEDYLSLILNRLCVLHEKTPVSEKVTKCCTESLVNRRP |
| | | CFSALTPDETYVPKAFDEKLFTFHADICTLPDTEKQIKKQTALVELLKHKPK |
| | | ATEEQLKTVMENFVAFVDKCCAADDKEACFAVEGPKLVVSTQTALA |

An animal protein can comprise a sheep protein (Table 4). A sheep protein can comprise a sheep muscle protein. A sheep protein can comprise a sheep blood protein. A sheep protein can comprise a sheep albumin protein. A sheep protein can comprise a sequence that has at least or about 70%, at least or about 75%, at least or about 80%, at least or about 90%, at least or about 91%, at least or about 92%, at least or about 93%, at least or about 94%, at least or about 95%, at least or about 96%, at least or about 97% at least or about 98% at least or about 99% or about 100% sequence identity to any one of SEQ ID NO: 94-108.

A food product may comprise a fragment or a truncation of a sheep protein. The food product may include a fragment or truncation of any one of SEQ ID Nos: 94-108. A sequence of the fragment or truncation may be identical or substantially identical to a portion of any one of SEQ ID Nos: 94-108. A sequence of the fragment or truncation may be at least about 70%, 80%, 90%, 95%, 98%, or more identical to a portion of a sequence of any one of SEQ ID Nos: 94-108. A length of the fragment or truncation may be at least about 10%, 15%, 20%, 30%, 40%, 50%, 60%, 70%, 80%, 90%, or more of a length of any one of SEQ ID Nos: 94-108. A length of the fragment or truncation may be less than or equal to about 90%, 80%, 70%, 60%, 50%, 40%, 30%, 20%, 15%, 10%, or less than a length of any one of SEQ ID Nos: 94-108.

TABLE 4
Sheep Proteins

| SEQ | | |
|---|---|---|
| ID NO | Description | Amino Acid Sequence |
| 94 | Sheep W5PJ69 | MPFGNTHNKHKLNFKAEEEYPDLSKHNNHMAKALTLEIYKKLRDKETPSG |
| | (Creatine kinase) | FTLDDVIQTGVDNPGHPFIMTVGCVAGDEESYTVFKDLFDPIIQDRHGGFKP |
| | | TDKHKTDLNHENLKGGDDLDPHYVLSSRVRTGRSIKGYALPPHCSRGERR |
| | | AVEKLSVEALNSLTGEFKGKYYPLKSMTEQEQQQLIDDHFLFDKPVSPLLL |
| | | ASGMARDWPDARGIWHNDNKSFLVWVNEEDHLRVISMEKGGNMKEVFR |
| | | RFCVGLQKIEEIFKKAGHPFMWNEHLGYVLTCPSNLGTGLRGGVHVKLAH |
| | | LSKHPKFEEILTRLRLQKRGTGGVDTAAVGSVFDVSNADRLGSSEVEQVQL |
| | | VVDGVKLMVEMEKKLEKGQSIDDMIPAQK |
| 95 | Sheep W5P9P1 | MMMVLQPEGLGTGEGPFAGGGRGGGEYMEQEEDWDRDLLLDPAWEKQQ |
| | (Actinin alpha 3) | RKTFTAWCNSHLRKAGTQIENIEEDFRNGLKLMLLLEVISGERLPRPDKGK |
| | | MRFHKIANVNKALDFIASKGVKLVSIGAEEIVDGNLKMTLGMIWTIILRFAI |
| | | QDISVEETSAKEGLLLWCQRKTAPYRNVNVQNFHTSWKDGLALCALIHRH |
| | | RPDLIDYAKLRKDDPIGNLNTAFEVAEKYLDIPKMLDAEDIVNTPKPDEKAI |
| | | MTYVSCFYHAFAGAEQAETAANRICKVLAVNQENEKLMEEYEKLASELLE |
| | | WIRRTVPWLENRVGEPSMSAMQRKLEDFRDYRRLHKPPRVQEKCQLEINF |
| | | NTLQTKLRLSHRPAFMPSEGKLVSDIANAWRGLEQAEKGYEDWLLSEIRRL |
| | | QRLQHLAEKFQQKASLHEAWTRGKEEMLSQRDYETASLQEVRALLRRHEA |
| | | FESDLAAHQDRVEHIAALAQELNELDYHEAASVNSRCQAICDQWDNLGTL |
| | | TQKRRDALERMEKLLETIDQLQLEFARRAAPFNNWLDGAVEDLQDVWLV |
| | | HSVEETQSLVTAHDQFKATLPEADRERGAILGIQGEIQKICQTYGLRPSSTNP |
| | | YIALTPQDINTKWDTVRKLVPSRDQMLQEELTRQQMNERLRRQFAAQANA |
| | | IGPWIQGKVEEGGRLAAGMAGSLEEQMAGLRQQEQNIINYKSNIDRLEGDH |
| | | QLLQESLVFDNKHTVYSMEHIRVGWEQLLTSIARTINEVENQVLTRDAKGL |
| | | SQEQLNEFRASFNHFDRKRNGMMEPDDFRACLISMGYDLGEVEFARIMTM |
| | | VDPNAAGVVTFQAFIDFMTRETAETDTAEQVVASFKILAGDKNYITAEELR |
| | | RELPAEQARRAPDRGAGAPAGALDYVAFSSALYGESDL |
| 96 | Sheep PYGM | MSRPLTDQEKRKQISVRGLAGVENVTELKKNFNRHLHFTLVKDRNVATPR |
| | (Glycogen | DYYFALAYTVRDHLVGRWIRTQQHYYEKDPKRIYYLSLEFYIGRTLQNTM |
| | phosphorylase) | VNLALENACDEATYQLGLDMEELEEIEEDAGLGNGGLGRLAACFLDSMAT |
| | | LGLAAYGYGIRYEFGIFNQKISGGWQMEEADDWLRYGNPWEKARPEFTLP |
| | | VHFYGRVEHTSQGAKWVDTQVVLAMPYDTPVPGYRNNVVNTMRLWSAK |
| | | APNDFNLKDFNVGGYIQAVLDRNLAENISRVLYPNDNFFEGKELRLKQEYF |
| | | VVAATLQDIIRRFKSSKFGCLDPVRTNFDAFPDKVAIQLNDTHPSLAIPELM |
| | | RILVDQERLEWEKAWEVTVKTCAYTNHTVLPEALERWPVHLIETLLPRHLQ |
| | | IIYEINQRFLNRVAAAFPGDVDRLRRMSLVEEGAVKRINMAHLCIAGSHAV |
| | | NGVARIHSEILKKTIFKDFYELEPHKFQNKTNGITPRRWLVMCNPGLAEVIA |
| | | ERIGEEYIADLDQLRKLLSYVDDESFIRDVAKVKQENKLKFSAYLEKEYKV |
| | | HINPNSLFDIQVKRIHEYKRQLLNCLHVITLYNRIKKEPNKFFVPRTVMIGGK |
| | | AAPGYHMAKMIIRLITAIGDVVNHDPVVGDRLRVIFLENYRVSLAEKVIPAA |
| | | DLSEQISTAGTEASGTGNMKFMLNGALTIGTMDGANVEMAEEAGEENFFIF |
| | | GMRVEDVERLDQKGYNAQEYYDRIPELRHIIDQLSSGFFSPKQPDLFKDIVN |
| | | MLMHHDRFKVFADYEEYVKCQERVSALYKNPREWTRMVIRNIATSGKFSS |
| | | DRTIAQYAREIWGVEPTRQRMPAPDEKI |
| 97 | Sheep | MPHQYPALTPEQKKELCDIAHRIVAPGKGILAADESTGSIAKRLQSIGTENTE |
| | A0A6P7DGM3 | ENRRFYRQLLLTADDRVNPCIGGVILFHETLYQKADDGRPFPQVIKAKGGV |
| | (Fructose- | VGIKVDKGVVPLAGTNGETTTQGLDGLSERCAQYKKDGADFAKWRCVLKI |
| | bisphosphate | GEHTPSSLAIMENANVLARYASICQQNGIVPIVEPEILPDGDHDLKRCQYVT |
| | aldolase) | EKVLAAVYKALSDHHIYLEGTLLKPNMVTPGHACTQKYSHEEIAMATVTA |
| | | LRRTVPPAVPGITFLSGGQSEEEASINLNAINKCPLLKPWALTFSYGRALQAS |
| | | ALKAWGGKKENLKAAQEEYVKRALANSLACQGKYTPSGKAGAAASESLFI |
| | | SNHAY |
| 98 | Sheep W5QD16 | ILLNKMAPKKDVKKPAAAAAPAPAPAPAPAPAPAPPKEEKIDLSAIKIEFSK |
| | (Myosin light | QQQDEFKEAFLLYDRTGDGKITLSQVGDVLRALGTNPTNAEVKKVMGNPS |
| | chain 1) | NEEMNAKKIEFEQFLPMLQAISNNKDQGTYEDFVEGLRVFDKEGNGTVMG |
| | | AELRHVLATLGEKMKEEEVEALMAGQEDSNGCINYEAFVKHIMSN |
| 99 | Fragment of | IGRLVTRAAFNTGKVDIVAINDPFIDLHYMVYMFQYDSTHGKFHGTVKAEN |
| | Sheep G3P | GKLVINGKAITIFQERDPANIKWGDAGAEYVVESTGVFTTMEKAGAHLKG |
| | (Glyceraldehyde- | GAKKVIISAPSADAPMFVMGVNHEKYNNTLKIVSNASCTTNCLAPLAKVIH |
| | 3-phosphate | DHFGIVEGLMTTVHAITATQKTVDGPSGKLWRDGRGAAQNIIPASTGAAKA |
| | dehydrogenase) | VGKVIPELNGKLTGMAFRVPTPNVSVVDLTCRLEKPAKYDEIKKVVKQASE |
| | | GPLKGILGYTEDQVVSCDFNSDTHSSTFDAGAGIALNDHFVKLISWYDNEF |
| | | GYSNRVVDLMVHMASKE |
| 100 | Sheep W5QI03 | MDAIKKKMQMLKLDKENALDRAEQAEADKKAAEDRSKQLEDELVSLQKK |
| | (Tropomyosin 1) | LKATEDELDKYSEALKDAQEKLELAEKKATDAEADVASLNRRIQLVEEEL |
| | | DRAQERLATALQKLEEAEKAADESERGMKVIESRAQKDEEKMEIQEIQLKE |
| | | AKHIAEDADRKYEEVARKLVIIESDLERAEERAELSEGQVRQLEEQLRIMDQ |
| | | TLKALMAAEDKYSQKEDKYEEEIKVLSDKLKEAETRAEFAERSVTKLEKSI |
| | | DDLEDELYAQKLKYKAISEELDHALNDMTSIESGSCQRRKP |
| 101 | Sheep W5PX04 | MPEAKPAAKKAPAGKDAAAKPAPKEAPPQEAPAAPSTEAPPEDQSPTAEEP |
| | (Myosin binding | TGVFLKKPDSVSVENGKDTVIVAKLNGKELPAKPAVKWFKGKWLELGSKS |
| | protein C2) | GARFSFKESHDAASNVYTVELHITKVVLGDRGDYRIEVKAKDFCDSCTFNI |
| | | DVESPRQDSAGQSLESFKRSSEAKSDTAGELDFSGLLKKRQVVEEEKKKKK |
| | | DDDDLGIPPEIWELLKGAKKSEYERIAFQYGITDLRGMLKRLKKAKVEVKK |
| | | SAAFTKKLDPAYQVDKGNKIKLVVEISDPDLPLKWFKNGQEIKPSSKYVFE |
| | | NVGKKRILTINKCTLADDAAYEVVVKDEKCFTELFVKVEPPVLIVTPLEDQ |
| | | QVFVGNRVEMAVEVSEEGAQVMWLKDGVELTREDSFKSRYRLKKDGKRH |
| | | ILIYAEVALEDKGHYKVMTNGDQCEAELIVEEKQLEVLQDIADLTVKASEQ |
| | | AVFKCEVSDEKVTGKWYKNGVEVRPSKRITISHTGRIHKLVIDDVRPEDEG |
| | | DYTFVPDGYALSLSAKLNFLGAGLSPLPPEPPKIHLDCSGQTSENSVVVVAG |
| | | NKLRLDVSITGEPRPVATWMKEDEVFTGTEGRVRIEQRGDISSFVIESAERG |
| | | DEGRYTIKVTNPAGEDVASILLKVVDVPDPPEAVRVTSVGEDWAVLVWEP |
| | | PKYDGGRPVTGYLLERKKKGSQRWMKLNFEVFTETTYESTNMIEGILYEM |
| | | RVFAVNAIGVSQPSMNTKPFMPIAPTSEPQHLTVEDVTDTTTTLKWRPPEKI |
| | | GAGGIDGYLIEYCVEGSDEWIPANTEPVERCGFTVKNLPTGAKITFRVVGV |
| | | NIAGRSQPATLAQPVTIREIVEQPKIRLPRHLRQTYVRKVGEHLNLVIPFQGK |
| | | PRPQVVWTKGGAPIDTNHVHVRTSDFDTVFFVRQAARSDSGEYELTVQIEN |
| | | MKDTATIRIRVVEKAGPPENVMVKEVWGTNALVEWQPPRDDGNSEITGYL |
| | | VQKADKKTMEWFTVYERNRHTSCTVSDLIMGNEYYFRVYSENICGLSDLP |
| | | GVSKNTARIVKTGMTLKLPEYKEHDFRTPPKFLTPLFDRVVVAGYAAALNC |
| | | AVRGHPKPKVVWMKNKMEIREDPKFLMTNHQGVLTLNIRRPSPFDSGTYS |
| | | CRAVNELGEALAECKLEVRGEP |
| 102 | Sheep W5P6W4 | MSLVAVPFYQKRHKHFDQSYRNIQTRYLLDEYAAKKKRASAQSSLQRSVT |
| | (Myomesin 2) | RKSSSQRESSRAALGETTLGETTLGRTTCPLCAKRMSSALEEEAQERKRRY |
| | | QSMVAAYGEAKRERYLSELAQLEEDVHVARTHAREELDRLALQHAGRHG |
| | | LHHAVDDRLAWERHWFEDRISRAPEILVRLRSHTVWERMSVKLCFTVQGF |
| | | PTPVVQWYKDGSLICQAGEPGKYRIDSKYGVHTLEINRADFDDTATYSAVA |
| | | TNVHGQVSTNAAVVVRRFRGDEEPFHSVGLPIGLPLSSVIPYTHFDVQFIEK |
| | | FGVTFRREGETLTLKCTLLVTPDLKRVQPRAEWYRDDVLLKESKWTKMFF |
| | | GEGQASLSFSHLNKDDEGLYTLRIVSRGGVTDHSAFLFVRDADPLVTGAPG |
| | | APMDVHCHDANRDYVIVTWKPPNTTKESPVIGYFIDRCEVGTDNWVQCND |
| | | APVKICKYPVTGLFEGRSYIFRVRAVNSAGISRPSRVSEAVAALDPVDLRRL |
| | | QAVHLEGEKDIVIYQEDLEGEVQIPGPPTNVHASETSRTYVVLSWDPPEPRG |
| | | KEPLMYFIEKSMVGSGSWQRVNAQTAVRSPRYAVFDLAEGKPYVFRVLSA |
| | | NKHGLSEPSEITAPIQAQDTIVVPSAPGRVLASRNTRTSVVVQWDRPKHEED |
| | | LLGYYVDCSVAGSNVWEPCNHKPIGYNRFVVHGLTTGEQYIFRVKAVNAV |
| | | GTSENSQESDVIKVQAALTVPSHPYGITLLNCDGHSMILGWKVPKFSGGSPI |
| | | LGYYIDKREAHHKNWHEVNSSPLKERILKVEGLTEGSLYEFKIAAANMAG |
| | | MGSQQTELFKCEAWTMPEPGPAYDLTFCEVRDTSLVLLWKPPVYSGSSPVS |
| | | GYFVDYKEEDSGEWLTVNEVTTPHRYLKVCDLHQGKTYVFRVRAVNASG |
| | | VGRPSDTSEPVLVEARPGTKEVSAGVDEEGNIYLSFDCPEVTDASHFTWCK |
| | | SYEDIADDDRFKVETAGDHSKLYFKNLDKEDLGTYSVSVSDTDGVSSSFVL |
| | | DEEELERLKALSNEIKNPTIPLKSELAYEIFDKGQVRFWLQAEHLSPDSSYRF |
| | | IINDREVADSETHRIKCDKSTGVIEMVMDRFTVDNEGTYTVQIQDGKAKSQ |
| | | SSLVLIGDAFKAVLEEAEFQRKEFLRKQGPHFAEYLHWDVTEECEVRLVCK |
| | | VANTKKETVFKWLKDDVLYETEKMPDLEKGVCELLIPKLSKKDHGEYKAT |
| | | LKDDRGQDVSTLEIAGKVYEDMILAMSRVCGASASPLKILCTPEGIRLQCF |
| | | MKYFTEEMKVNWYHKDAKIASSEHMRIGGSEEMAWLQICEPTEKDKGKY |
| | | TFEILDGKKNHQRSLDLSGQAFDEAFAEFQQLKAAAFAEKNRGKVIGGLPD |
| | | VVTIMEGKTLNLTCTVFGNPDPEVVWFKNDKDIELSDHFSVKVEQAKYVS |
| | | MTIKGVTSEDSGKYSMHVKNKYGGEKIDVTVSVYKHGEEIPDVVLPQQAK |
| | | PKLLPAAAPSPAH |
| 103 | Sheep W5P663 | MAMQKIFAREILDSRGNPTVEVDLHTAKGRFRAAVPSGASTGIYEALELRD |
| | (Phosphopyruvate | GDKSRYLGKGVLKAVEHINKTLGPALLEKKLSVVDQEKVDKFMIELDGTE |
| | hydratase) | NKSKFGANAILGVSLAVCKAGAAEKGVPLYRHIADLAGNPELILPVPAFNVI |
| | | NGGSHAGNKLAMQEFMILPVGASSFREAMRIGAEVYHHLKGVIKAKYGKD |
| | | ATNVGDEGGFAPNILENNEALELLKTAIQAAGYPDKVVIGMDVAASEFYRN |
| | | GKYDLDFKSPDDPARHISGEKLGELYKSFIKNYPVVSIEDPFDQDDWATWT |
| | | SFLSGVNIQIVGDDLTVTNPKRIAQAVEKKACNCLLLKVNQIGSVTESIQAC |
| | | KLAQSNGWGVMVSHRSGETEDTFIADLVVGLCTGQIKTGAPCRSERLAKY |
| | | NQLMRIEEALGDKAVFAGRKFRNPKAK |
| 104 | Sheep W5P7V0 | QAPKKAKRRAAAEGGSSNVFSMFDQTQIQEFKEAFTVIDQNRDGIIDKEDL |
| | (Myosin light | RDTFAAMGRLNVKNEELDAMMKEASGPINFTVFLTMFGEKLKGADPEDVI |
| | chain) | TGAFKVLDPEGKGTIKKQFLEELLTTQCDRFSQEEIRNMWAAFPPDVGGNV |
| | | DYKNICYVITHGDAKDQE |
| 105 | Sheep W5PPG6 | MSSDQEMAIFGEAAPYLRKSEKERIEAQNKPFDAKTSVFVADPKESFVKAT |
| | (Myosin heavy | VQSREGGKVTAKTEAGATVTVKEDQVFPMNPPKFDKIEDMAMMTHLHEP |
| | chain 1) | AVLYNLKERYAAWMIYTYSGLFCVTVNPYKWLPVYNAEVVAAYRGKKR |
| | | QEAPPHIFSISDNAYQFMLTDRENQSILITGESGAGKTVNTKRVIQYFATIAV |
| | | TGEKKKEEPTSGKMQGTLEDQIISANPLLEAFGNAKTVRNDNSSRFGKFIRI |
| | | HFGTTGKLASADIETYLLEKSRVTFQLKAERSYHIFYQIMSNKKPELIEMLLI |
| | | TTNPYDYAFVSQGEITVPSIDDQEELMATDSAIEILGFTSDERVSIYKLTGAV |
| | | MHYGNLKFKQKQREEQAEPDGTEVADKAAYLQGLNSADLLKALCYPRVK |
| | | VGNEFVTKGQTVEQVYNAVGALAKAIYDKMFLWMVTRINQQLDTKQPRQ |
| | | YFIGVLDIAGFEIFDFNSLEQLCINFTNEKLQQFFNHHMFVLEQEEYKKEGIE |
| | | WEFIDFGMDLAACIELIEKPMGIFSILEEECMFPKATDTSFKNKLYEQHLGKS |
| | | NNFQKPKPAKGKAEAHFSLIHYAGTVDYNITGWLDKNKDPLNETVVGLYQ |
| | | KSSVKTLAFLFSGAASAEAEGGGAKKGGKKKGSSFQTVSALFRENLNKLM |
| | | TNLRSTHPHFVRCIIPNETKTPGAMEHELVLHQLRCNGVLEGIRICRKGFPSR |
| | | ILYADFKQRYKVLNASAIPEGQFIDSKKASEKLLGSIDVDHTQYKFGHTKVF |
| | | FKAGLLGLLEEMRDEKLAQLITRTQARCRGFLARVEYQRMVERRESIFCIQ |
| | | YNVRAFMNVKHWPWMKLYFKIKPLLKSAETEKEMANMKEEFEKTKEELA |
| | | KSEAKRKELEEKMVTLTQEKNDLQLQVQSEADALADAEERCDQLIKTKIQL |
| | | EAKIKEVTERAEDEEEINAELTAKKRKLEDECSELKKDIDDLELTLAKVEKE |
| | | KHATENKVKNLTEEMAGLDETIAKLTKEKKALQEAHQQTLDDLQAEEDK |
| | | VNTLTKAKTKLEQQVDDLEGSLEQEKKLRMDLERAKRKLEGDLKLAQEST |
| | | MDIENDKQQLDEKLKKKEFEMSNLQSKIEDEQALGMQLQKKIKELQARIEE |
| | | LEEEIEAERASRAKAEKQRSDLSRELEEISERLEEAGGATSAQIEMNKKREA |
| | | EFQKMRRDLEEATLQHEATAAALRKKQGGVGEQLDNLQRVKQKLEKEKS |
| | | EMKMEIDDLASNMETVSKAKGNLEKMCRTLEDQLSELKTKEDEQQRLIND |
| | | LTTQRARLQTESGEFSRQLDEKDALVSQLSRGKQAFTQQIEELKRQLEEEIK |
| | | AKSALAHALQSARHDCDLLREQYEEEQEAKAELQRALSKANSEVAQWRT |
| | | KYETDAIQRTEELEEAKKKLAQRLQDAEEHVEAVNAKCASLEKTKQRLQN |
| | | EVEDLMIDVERTNAACAALDKKQRNFDKILSEWKQKYEETHAELEASQKE |
| | | SRSLSTELFKIKNAYEESLDQLETLKRENKNLQPTEEISDLTEQIAEGGKRIH |
| | | ELEKVKKQVEQEKSEIQAALEEAEASLEHEEGKILRIQLELNQVKSEIDRKIA |
| | | EKDEEIDQLKRNHIRVVESMQSTLDAEIRSRNDAIRLKKKMEGDLNEMEIQ |
| | | LNHANRMAAEALKNYRNTQAILKDTQLHLDDALRGQEDLKEQLAMVERR |
| | | ANLLQAEIEELRATLEQTERSRKIAEQELLDASERVQLLHTQNTSLINTKKK |
| | | LETDITQIQGEMEDIIQEARNAEEKAKKAITDAAMMAEELKKEQDTSAHLE |
| | | RMKKNLEQTVKDLQHRLDEAEQLALKGGKKQIQKLEARVRELEAEVESEQ |
| | | KRNVETVKGLRKHERRVKELTYQTEEDRKNILRLQDLVDKLQAKVKSYKR |
| | | QAEEAEEQSNVNLSKFRKLQHELEEAEERADIAESQVNKLRVKSREVHTKII |
| | | SEE |
| 106 | Sheep W5PFV9 | MADDEEYEEVVEYYTEETVYEEVPGETITEFYETTTTRTSDYEQSDTSRPAL |
| | (Nebulin) | AKPVPEKPVERKRVIRKKVDPSKFMTPYIAHSQKMQDLFSTNKYKENYEK |
| | | AKGQPYAITTDTPELRRIKKVQDQLSEVKYRTDGDVAKTICHVDEKAKDIE |
| | | HAKKVSQQVSKVLYKQNWEDTKDKYLLPPDAPELVQAIKNTAMFSKKLY |
| | | TEDWEADKTLFYPYNDSPELRRVAQAQKALSDITYKKGLAEQQTQFTSLPD |
| | | PPDIEFAKKVTNQVSKQKYKEDYENKVKGKWSETPCFEIATARMNSQNIST |
| | | KKYQEDFENTKDQIYFMQTETPEYKVNKQAGVAASKVKYKQDYEKNKGK |
| | | ADYNVLPASENPLLRQLKAAGDALSDKLYKENYEKTKAKSINYCETPKFKL |
| | | DTVLHNFSSDTKYKDSYLKNILGHYVGSYEDPYHTHCMRVSAQNSDKNYK |
| | | AEYEEDRGKGFFPQTITQEYEAIKKLDQCKDHTYKVHPDKTKFTQVTDSPV |
| | | LVQAQVNSKQLSDLNYKAKHENEKFKCHIPPDTPAFIQHKVNAYNLSDNIY |
| | | KHDWEKTKAKKFDIKVDAIPLLAAKANTKIASDVMYKKDYEKSKGKMIG |
| | | ALSINDDPKMLHSLKTAKNQSDRLYKENYEKTKAKSMNYCETPKYQLDTL |
| | | LKNFSEAKYKDSYVQNVLGHYIGSFEDPYREHCMKVAAQNSDKNYKAEY |
| | | EEDKGKCYFPQTITQEYEAIKKLDQCKDHTYKVHPDKTKFTAVTDTPVLLQ |
| | | AQLNTKQLSDLNYKAKHEGEKFKCHIPADAPQFIQHRVNAYNLSDNIYKH |
| | | DWEKTKAKKFDIKVDAIPLLAAKANTKIASDVMYKKDYEKSKGKMIGALS |
| | | INDDPKMLHSLKTAKNQSDREYRKDYEKSKTIYTAPLDMLPLTHAKKSQEI |
| | | ASDVDYKHLLHNYSYPPDSVNVDLAKKAYALQSDVEYKADYNSWMKGC |
| | | GWVPFGSLEMEKAKRASDILNEKKYRQHPDTLKFTSIEDAPIIVQSKINQAQ |
| | | RSDVAYKAKGEEVIHKYSLPADLPQFIQAKVNAYNISENMYKADLKDLSK |
| | | KGYDLRIDAIPIKAAKAARQAASDVQYKKDYEKAKGKMVGFQSLQDDPKL |
| | | VHYMNVAKIQSDREYKKDYEKTKTRHNTPHDMVNIVAAKKAQDVASNV |
| | | NYKHSLHHYTYLPDAMDLELSKNMMHIQSDNVYKEDYNNWMKGIGWIPI |
| | | GSLEVEKVKKAGDALNEKKYRQHPDTLKFTSIVDSPVMVQAKQNTQQVSD |
| | | ILYKAKGEDVKHKYTMSPDLPQFLQAKCNAYNLSDVCYKRDWHDLIAKG |
| | | TNVLGDAIPITAAKASRNIASDYKYKEAYEKAKGKQVGFRSLQDDPKLVH |
| | | YMNVAKLQSDREYKKNYENTKTSYHTPGDMVSITAAKMAQDVATNVNY |
| | | KQPIHHYTYLPDALSLEHIRNVNQIQSDKVYKDEYNHFFKGIGWIPIGSLEV |
| | | EKVKKAGDALNEKKYRQHPDTIKFTSVPDSMGMVLAQHNTKQLSDLNYK |
| | | VEGEKVKHKYTLDPDVPQFIQAKVNAFNMSDSHYKADWKKTLAKGYDLR |
| | | PDAIPIVAAKSSRNIASDFKYKEGYEKAKGKQIGFRSLQDDPKLVHFMNVA |
| | | KMQSDREYKKGYEASKTKYHTPLDMLSVTAAKKSQEVATNTNYKQPFHH |
| | | YTLLPDALNVEHSRNAMQIQSDNLYKSDFTSWMKGIGWMPLESLEVEKAK |
| | | KAGEILSEKKYRQHPEKLKFTYAMDTMEQALNKSNKLIMDKRRYTEKWN |
| | | KDKTTIHVMPDTPDILLSRVNQITMSDKLYKAGWEEEKKKGYDLRPDAIPI |
| | | KAARASRDIASDYKYKQAYEQAKGKQIGFRSLEDDPKLVHFMQVAKMQS |
| | | DREYKKAYEKSKTSFHTPVDMLSVVAAKKSQEVATNANYRNVIHNYNML |
| | | PDAMSLELAKNMMQIQSDNQYKADYADFMKGIGWLPLGSLEAEKNKKA |
| | | MEIISEKKYRQHPDTLKYSTLMDSMNMVLAKNNAKIMNEHLYKQAWEAD |
| | | KTKVHIMPDIPQIILAKANAINMSDKLYKLSLEEAKKKGYDLRTDAIPIKAA |
| | | KASRDIASDYKYKHNYEKERGKMVGFRSLEDDPKLVHSMQVAKMQSDRE |
| | | YKKNYEKTKTSYHTPADMLSVTAAKDAQANITNTNYKHLIHKYILLPDAM |
| | | NIQLSKNMNRLQSDNEYKQDYNEWYKGLGWSPAGSLEVEKAKRATEYAS |
| | | DQKYRQHPSNFQFSKLNDSMDMVLAKQNAHTMNKYLYTVDWNKDKTKI |
| | | HVMPDTPDILQAKQNQTIYSQKLYKLGWEEALKKGYDLPVDAISVQAAKA |
| | | SRDIVSDFKYKQGYRKQIGHHIGFRSLQDDPKLVLSMNVAKMQSEREYKK |
| | | DFEKWKTKFSSPVDMLGVVLAKKCQALVSDVDYKNYLHGWTCLPDQND |
| | | VIHAKKAYDLQSENLYKSDLEWLKGIGWSPLGSLEAEKNKRASEIISEKKY |
| | | RQPPDRNKFTSIPDAMDIVLAKTNAKNRSDILYREAWDKDKTQVHIMPDTP |
| | | DIILAKANLINASDKFYRMGYEELRKKGYDLPVDAIPIKAAKASREIASEYK |
| | | YKEGFRKQLGHHIGARNIKDDPKMMWSMHVAKIQSDREYKKDFEKWKTK |
| | | FSSPVDMLGVVLAKKCQTLVSDIDYKNYLHQWTCLPDQSDVIHARRAYDL |
| | | QSDNLYKSDLQWLKGIGWLPSGSLEDEKNKRATQILSDHVYRQHPDKFKFS |
| | | SLMDSIPMVLAKNNAITMNHRLYTEAWDKDKTTVHIMPDTPGVLLAKQNQ |
| | | INYSEKLYKLGLDEAKRKGYDMRIDAIPIRAAKASRDIASEFKYKEGYRKQ |
| | | LGHHIGARAIHDDPKMMWSMHVAKIQSDREYKKDFEKWKTKFSSPVDML |
| | | GVVLAKKCQTLVSDIDYKNYLHQWTCLPDQSDVIHARQAYDLQSDNVYK |
| | | SDLQWLKGIGWVPIGSLDVEKCKRASEILSDKLYRQPPDKFKFTSVTDSLEQ |
| | | VLAKNNAINMNKRLYTEAWDKDKTQVHIMPDTPEITLARMNKVNYSESLY |
| | | KLAHEEAKKKGYDLRSDAIPIVAAKASRDIISDYKYKDGYRKQLGHHIGAR |
| | | DIQDDPKMMWSMHVAKIQSDREYKKDFEKWKTKFSSPVDMLGVVLAKKC |
| | | QTLVSDIDYKNYLHQWTCLPDQSDVIHARRAYDLQSDNVYKSDLQWLRGI |
| | | GWVPIGSVDVVKCKRAAEILSDNLYRQPPDTLKFTSVPDSLEQVLAKNNAI |
| | | NMNKRLYTEAWDKDKTQVHIMPDTPEIMLARQNKLNYSETLYKLANEEA |
| | | KKKGYDLRSDAIPIVAAKASRDIISDYKYKDGYRKQLGHHIGARDIKDDPK |
| | | MMWSMHVAKIQSDREYKKDFEKWKTKFSSPVDMLGVVLAKKCQTLVSDI |
| | | DYKNYLHQWTCLPDQSDVIHARQAYDLQSDNIYKSDLQWLRGIGWVPIGS |
| | | VDVVKCKRAAEILSDNLYRQRPDTLKFTSVPDSLEQVLAKNNAINMNKRL |
| | | YTEAWDKDKTQIHMMPDTPEITLARQNKINYSENLYRQAMEEAKKEGYDL |
| | | RSDAIPIVAAKASREIASDYKYKEAYRKQLGHHIGARAIHDDPKMMWSVH |
| | | VAKMQSDREYKKDFEKYKTRFSSPVDMLGIVLAKKCQTLVSDVDYKHPLH |
| | | EWTCLPDQNDIIHARKAYDLQSDNLYKSDLEWLKGIGWVPIGSFDVVKAK |
| | | RAGEILSDNIYRQRPDTLKFTSVTDSPEQVLAKNNAINMSKRLYTEAWDND |
| | | KKTIHVMPDTPEIMLAKLNRINYSDKLYKLALEESKKEGYDLRLDAIPIQAA |
| | | KASREIASDYKYKEGYRKQLGHHIGARNIKDDPKMMWSIHATKLQSDREY |
| | | KKDFEKWKTKFSSPVDMMGLVQAKKCQILVSDIDYKNLLHEWICLPDQND |
| | | VIQARKAYDLQSDAIYKADLEWLRGIGWVPIGSVGVENVKRAGEILSERKY |
| | | RQPAVQLKFTSITDTPEIVLAKNNALNMSKHLYTEAWDADKTSIHVMPDTP |
| | | EILLAKSNSANISHKLYTKGWDESKMKDYDLRADAISIKSAKASRDIASDY |
| | | KYKEAYEKQKGHHIGARSVEDDPKIMCAMNAGKIQSEREYKKEFQKWKT |
| | | KFSSPVDMLGILQAKKCQILVSDIDYRNYLHHWTCLPDQNDIIQARKAYDL |
| | | QSDAIYKADLEWLRGIGWMPEGSPEVLRVKNAQHIFRDSVYRTPVVKLKY |
| | | TSIVDTPEVVLAKSNAENISIPKYREVWDKDKTSIHIMPDTPEINLARTNALN |
| | | VSNKLYREGWDEMKMSCDVRLDAIPIQAAKASREIASDYKYKLDHEKQKG |
| | | HYVGTRTARDDNKIRWALIAGKIQNEREYRMHWAKWKSKFQSPPDMLSIE |
| | | HSKNSQTLVSDIDYRHYLHQWTCMPDQNDVIQARKAYDLQSDAIYKADLE |
| | | WLRGIGWMPADSVSVNHAKHMGDIFNEKKYRTKIETLNFTPVDDRVDYVT |
| | | AKHSGEILNDIKYRKDWNDTKSKYTLTETPQLHAAQEAARILDQYLYKES |
| | | WEKQKATGYILPPDAVPFVHAHHSGDVQSELKYKAEHVKQKGHYVGVPT |
| | | MRDDPKLVWFEHAGQIQNDRLYKESYHKTKAKIHIPPDMVSVLAAKEGQA |
| | | LASDIDYRTYLHQWICHPDQNDVIQARKAYDLQSDNIYKADLEWLRGIGW |
| | | MPLDSVDHVRVTRNQEMVNQIKYKKDALANYPNFTSVVDPPEIVLAKINA |
| | | VNQSDVKYKETFNKLIKGKYIFSPDTPYITHSKDMEKLYSTILYKRAWEGT |
| | | KAYGYTLDERYIPIVGAKHADFVNSELKYKETYEKLKGHYLAGKEISEFPN |
| | | VVHCLDFQKMRSLLNYRRHYEDTKANVHIPQDMMNHVLAKRCQYILSDL |
| | | EYRHYFHQWTSLPEEPNVVRVRHAQEILSDNVYKDDLNWLKGIGCYVWD |
| | | TPQILHAKKSYDLQSQILYTAAGKENLKNYNLVTDTPLYVTALQSGINASE |
| | | VKYKENYHQIKDKYTTVLETVDYDRTRNLKDLFSSNLYKEAWDKVKATS |
| | | YILPPNTVSLTHAKNQKHMASYVKYREEYDKFKALYTLPRSVEDDPNTAR |
| | | CLRVGKFNIDRLYRSVYEKNKMNIHIVPDMVEMVTAKDSQKKVSEIDYRL |
| | | HLHEWICHPDLQVNSHVRKVTDQISDIVYKDDLTWLKGIGCYVWDTPEILH |
| | | AKHAYDLRNDIKYKAHVLKTRNNYKLVTDTPVYVQAVKSGKQLSDAVYH |
| | | YDYVHSIRGRVAPTTKTVDLDRALHAYKLQSENLYRKAGLHALPTGYRLP |
| | | VDTPHFKHTKDTRYMSSYFKYKEVYEHMKAYGYTLGPNDVPFVNVRRVN |
| | | NITSERLYRQLYHKLKDKIHTTPDTPEIRQVKKTQEAVSELIYKSDFFKMQG |
| | | LRKLWSHYLYTDKARKMRDKYQVVLDTPEYRTVQELKTHLSELVYRASG |
| | | RKQKSIFTSVPDTPDLIRAKRGQKLQSQYLYVELATKERPHHHAGNQTTAL |
| | | KHARHVKDMVSENKYKIQYEKMKDKYTPVPDTPILIRAKRAYWNASDLR |
| | | YKETFQKTKGRYHTVKDALDIVYHRTVTDHISKIKYKENYMSQLGIWRSIP |
| | | DRPEHFHHRAVTDAVSDVKYKADLTWLKGIGCYAYDTPDFTLAEKNKTLY |
| | | SKYKYKEVFERTKSNFKYVADCPINRHFKFATQLMNEKKYRADYEQRKDK |
| | | YHLVVDEPRHLLAKIAGDQISQIRYRKNYEKTKDKFTSIVDTPEHLRTTKVN |
| | | KQISDILYKLEYNKAKPRGYTTIHDTPMLLHVRKVKDEVSDLKYKEVYQRT |
| | | KSNCTIEPDAVHIKAAKDAYKVNTNLDYKKQYEATKAYWKWTPDRPDFIQ |
| | | AAKSTLQQSDFEYKLDREYLKGCKLSVTDDKDMVLALKNSIIESDLKYKEK |
| | | HVKERGSCHAVPDTPQILLAKTVSSLVSENKYKQYVKKHLAQGSYTTLPET |
| | | RDTIHVKEVTKNVSDTNYKKKFVKEKGKSNYSIMLEPPDVKHAMDVAKK |
| | | QSNMKVAYKKDAKENLHYTTVADRPDIKKATQAAKQASEVEYRAKHRKE |
| | | GSHGLSMLGRPDIEMAKKAAKLSSQVKYRENFNKEKGKTPKYNPKDSQLY |
| | | KVMKDANTLASEVKYKADLKKLHKPVTDMKESLIMNHVLNTSHLASSYQ |
| | | YKKNYEKSKGHYHTIPDNLEQLHLKEATELQSIVKYKEKYEKERGKPMLD |
| | | FETPTYITAKESQQMQSGKEYRRDYEESIKGRNLTGLEVTPALLHVKYATKI |
| | | ASEKEYRKDLEESIRGKGLSEMEDTPDMLRAKNATQILNEKEYKRDLELEV |
| | | KGRGLNAMANETPDFLRARNATDIASQIKYKQSAEMEKANFTSVVDTPEII |
| | | HAQQVKNLSSQKKYKEDAEKCMSYYETVLDTPEMQRVRENQKNFSLLQY |
| | | QYDLKNSKGKITVVQDTPEILRVKENQKNFSSVLYKEDVSPGTAIGKTPEM |
| | | MRVKQTQDHISSVKYKEVIGQGTPIPDLPEVKRVKQTQKHISSVMYKENLG |
| | | TGIPTPVTPEIERVKRNQENFSSVLYKENLGKGTPTPITPEMERVKRNQENFS |
| | | SILYKENLSKGTPLPVTPEMERVKRNQENFSSVLYKENVGKGTPTPVTPEM |
| | | QRVKRNQENISSVLYKENLGKATPTPFTPEMERVKRNQENFSSVLYKENMR |
| | | KATPTPVTPEMERAKRNQENISSVLYSDSFRKQIQGKAAYVLDTPEMRRVR |
| | | ETQRHISTVKYHEDFEKHKGCFTPVVTDPITERVKKNMQDFSDISYRGIQRK |
| | | VVEMEQKRNDQDQETVTGLRVWRTNPGSVFDYDPAEDNIQSRSLHMINAQ |
| | | AQRRSREQSRSASGLSVSGGEEKSEHSEAAHLSTYSDGGVFFSATSTGYKH |
| | | ARTTELPQQRSSSVATQQTTVSSIPSHPSTAGKIFRAMYDYMAADADEVSF |
| | | KDGDAIVNVQAIDEGWMYGTVQRTGRTGMLPANYVEAI |
| 107 | Sheep W5PIU1 | MSSDQEMAIFGEAAPYLRKSEKERIEAQNKPFDAKTSVFVADPKESFVKAT |
| | (Myosin-4) | VQSREGGKVTAKTEAGATVTVKEDQVFPMNPPKFDKIEDMAMMTHLHEP |
| | | GVLYNLKERYAAWMIYTYSGLFCVTVNPYKWLPVYNAEVVTAYRGKKRQ |
| | | EAPPHIFSISDNAYQFMLTDRENQSILITGESGAGKTVNTKRVIQYFATIAVT |
| | | GEKKKEEPTSGKMQGTLEDQIISANPLLEAFGNAKTVRNDNSSRFGKFIRIH |
| | | FGTTGKLASADIETYLLEKSRVTFQLKAERSYHIFYQIMSNKKPELIEMLLIT |
| | | TNPYDYAFVSQGEITVPSIDDQEELMATDSAIEILGFTSDERVSIYKLTGAVM |
| | | HYGNLKFKQKQREEQAEPDGTEVADKAAYLQSLNSADLLKALCYPRVKV |
| | | GNEFVTKGQTVQQVYNAVGALAKAVYEKMFLWMVTRINQQLDTKQPRQ |
| | | YFIGVLDIAGFEIFDFNSLEQLCINFTNEKLQQFFNHHMFVLEQEEYKKEGIE |
| | | WEFIDFGMDLAACIELIEKPMGIFSILEEECMFPKATDTSFKNKLYEQHLGKS |
| | | NNFQKPKPAKGKAEAHFSLVHYAGTVDYNIAGWLDKNKDPLNETVVGLY |
| | | QKSAMKTLAFLFAGGPSAEVHVSEGGGGKKGGKKKGSSFQTVSALFRENL |
| | | NKLMTNLRSTHPHFVRCIIPNETKTPGAMEHELVLHQLRCNGVLEGIRICRK |
| | | GFPSRILYADFKQRYKVLNASAIPEGQFIDSKKASEKLLGSIDIDHTQYKFGH |
| | | TKVFFKAGLLGILEEMRDEKLAQLITRTQAICRGFLTRVEFRKMMERSRESI |
| | | FCIQYNVRAFMNVKHWPWMKLYFKIKPLLKSAETEKEMANMKEEFEKTK |
| | | EELAKSEAKRKELEEKMVTLMQEKNDLQLQVQSEADGLADAEERCDQLIK |
| | | TKIQLEAKIKELTERAEDEEEINAELTAKKRKLEDECSELKKDIDDLELTLAK |
| | | VEKEKHATENKVKNLTEEMAGLDENIAKLTKEKKALQEAHQQTLDDLQA |
| | | EEDKVNTLTKSKTKLEQQVDDLEGSLEQEKKLRMDLERAKRKLEGDLKLA |
| | | QESTMDIENDKQQLDEKLKKKEFEMSNLQSKIEDEQALGMQLQKKIKELQ |
| | | ARIEELEEEIEAERASRAKAEKQRSDLSRELEEISERLEEAGGATSAQIEMNK |
| | | KREAEFQKMRRDLEEATLQHEATAAALRKKHADSVAELSEQIDNLQRVKQ |
| | | KLEEEGSNLTVQALDIISNFKIINRYYYIEGNLEKMCRTLEDQLSEVKSKEEE |
| | | HQRLINELSAQKARLHTESGEFSRQLDEKDALVSQLSRGKQAFTQQIEELKR |
| | | QLEEETKAKSALAHALQSARHDCDLLREQYEEEQEAKAELQRSMSKANSE |
| | | VAQWRTKYETDAIQRTEELEEAKKKLAQRLQEAEEHVEAVNAKCASLEKT |
| | | KQRLQNEVEDLMLDVERSNAACAALDKKQRNFDKVLSEWKQKYEETQAE |
| | | LEASQKESRSLSTELFKVKNAYEESLDQLETLKRENKNLQQEISDLTEQIAE |
| | | GGKHIHELEKVKKQIEQEKGELQAALEEAEASLEHEEGKILRIQLELNQVKS |
| | | EIDRKIAEKDEEIDQLKRNHLRVVESMQSTLDAEIRSRNDALRIKKKMEGDL |
| | | NEMEIQLNHANRQATEATKNLRNTQGVLKDTQLHLDDAIRGQDDLKEQLA |
| | | MVERRANLMQAEIEELRASLEQTERSRRVAEQELLDASERVQLLHTQNTSL |
| | | INTKKKLETDITRVHGSVVGSGIISKHLTFVYISVGCAFQAAMMAEELKKEQ |
| | | DTSAHLERMKKNLEQTVKDLQHRLDEAEQLALKGGKKQIQKLEARVRELE |
| | | NEVENEQKRNAEAVKGLRKHERRVKELTYQTEEDRKNVLRLQDLVDKLQ |
| | | SKVKAYKRQAEEAEEQANVNLSKFRKLQHELEEAEERADIAESQVNKLRV |
| | | KSREVHTKVISEE |
| 108 | Sheep ALBU | MKWVTFISLLLLFSSAYSRGVFRRDTHKSEIAHRENDLGEENFQGLVLIAFS |
| | (Albumin) | QYLQQCPFDEHVKLVKELTEFAKTCVADESHAGCDKSLHTLFGDELCKVA |
| | | TLRETYGDMADCCEKQEPERNECFLNHKDDSPDLPKLKPEPDTLCAEFKAD |
| | | EKKFWGKYLYEVARRHPYFYAPELLYYANKYNGVFQECCQAEDKGACLL |
| | | PKIDAMREKVLASSARQRLRCASIQKFGERALKAWSVARLSQKFPKADFTD |
| | | VTKIVTDLTKVHKECCHGDLLECADDRADLAKYICDHQDALSSKLKECCD |
| | | KPVLEKSHCIAEVDKDAVPENLPPLTADFAEDKEVCKNYQEAKDVFLGSFL |
| | | YEYSRRHPEYAVSVLLRLAKEYEATLEDCCAKEDPHACYATVFDKLKHLV |
| | | DEPQNLIKKNCELFEKHGEYGFQNALIVRYTRKAPQVSTPTLVEISRSLGKV |
| | | GTKCCAKPESERMPCTEDYLSLILNRLCVLHEKTPVSEKVTKCCTESLVNRR |
| | | PCFSDLTLDETYVPKPFDEKFFTFHADICTLPDTEKQIKKQTALVELLKHKP |
| | | KATDEQLKTVMENFVAFVDKCCAADDKEGCFVLEGPKLVASTQAALA |

An animal protein can comprise a pig protein (Table 5). A pig protein can comprise a pig muscle protein. A pig protein can comprise a pig blood protein. A pig protein can comprise a pig albumin protein. A pig protein can comprise a sequence that has at least or about 70%, at least or about 75%, at least or about 80%, at least or about 90%, at least or about 91%, at least or about 92%, at least or about 93%, at least or about 94%, at least or about 95%, at least or about 96%, at least or about 97% at least or about 98% at least or about 99% or about 100% sequence identity to any one of SEQ ID NO: 109-123.

A food product may comprise a fragment or a truncation of a pig protein. The food product may include a fragment or truncation of any one of SEQ ID Nos: 109-123. A sequence of the fragment or truncation may be identical or substantially identical to a portion of any one of SEQ ID Nos: 109-123. A sequence of the fragment or truncation may be at least about 70%, 80%, 90%, 95%, 98%, or more identical to a portion of a sequence of any one of SEQ ID Nos: 109-123. A length of the fragment or truncation may be at least about 10%, 15%, 20%, 30%, 40%, 50%, 60%, 70%, 80%, 90%, or more of a length of any one of SEQ ID Nos: 109-123. A length of the fragment or truncation may be less than or equal to about 90%, 80%, 70%, 60%, 50%, 40%, 30%, 20%, 15%, 10%, or less than a length of any one of SEQ ID Nos: 109-123.

TABLE5
Pig Proteins

| SEQ | | |
|---|---|---|
| ID NO | Description | Amino Acid Sequence |
| 109 | Pig KCRM | MPFGNTHNKYKLNFKAEEEYPDLSKHNNHMAKALTLEIYKKLRDKETPSGF |
| | (Creatine kinase | TLDDVIQTGVDNPGHPFIMTVGCVAGDEESYVVFKDLFDPIIQDRHGGYKPT |
| | M-type) | DKHKTDLNHENLKGGDDLDPNYVLSSRVRTGRSIKGYTLPPHCSRGERRAV |
| | | EKLSVEALNSLTGEFKGKYYPLKSMTEQEQQQLIDDHFLFDKPVSPLLLASG |
| | | MARDWPDARGIWHNDNKSFLVWVNEEDHLRVISMEKGGNMKEVFRRFCV |
| | | GLQKIEEIFKKAGHPFMWNEHLGYVLTCPSNLGTGLRGGVHVKLAHLSKHP |
| | | KFEEILTRLRLQKRGTGGVDTAAVGSVFDVSNADRLGSSEVEQVQLVVDGV |
| | | KLMVEMEKKLEKGQSIDDMIPAQK |
| 110 | Pig | MMMVMQPEGLGTREGPFAGGSGGGGEYMEQEEDWDRDLLLDPAWEKQQ |
| | A0A480REY2 | RKTFTAWCNSHLRKAGTQIENIEEDFRNGLKLMLLLEVISGERLPRPDKGKM |
| | (Actinin alpha 3) | RFHKIANVNKALDFIASKGVKLVSIGAEEIVDGNLKMTLGMIWTIILRFAIQDI |
| | | SVEETSAKEGLLLWCQRKTAPYRNVNVQNFHTSWKDGLALCALIHRHRPDL |
| | | IDYAKLRKDDPIGNLNTAFEVAEKYLDIPKMLDAEDIVNTPKPDEKAIMTYV |
| | | SCFYHAFAGAEQAETAANRICKVLAVNQENEKLMEEYEKLASELLEWIRRT |
| | | VPWLENRVGEPSMSAMQRKLEDFRDYRRLHKPPRVQEKCQLEINFNTLQTK |
| | | LRLSHRPAFMPSEGKLVSDIANAWRGLEQVEKGYEDWLLSEIRRLQRLQHL |
| | | AEKFQQKASLHEAWTRGKEDMLSQRDYESASLQEVRALLRRHEAFESDLA |
| | | AHQDRVEHIAALAQELNELDYHEAASVNSRCQAICDQWDNLGTLTQKRRD |
| | | ALERMEKLLETIDQLQLEFARRAAPFNNWLDGAVEDLQDVWLVHSVEETQS |
| | | LVTAHEQFKATLPEADRERGAILGIQGEIQKICQTYGLRPSSTNPYIALSPQDI |
| | | NTKWDTVRKLVPSRDQMLQEELTRQQVNERLRRQFAAQANAIGPWIQGKV |
| | | EEVGRLAAGMAGSLEEQMAGLRQQEQNIINYKSNIDRLEGDHQLLQESLVF |
| | | DNKHTVYSMEHIRVGWEQLLTSIARTINEVENQVLTRDAKGLSQEQLNEFR |
| | | ASFNHFDRKRNGMMEPDDFRACLISMGYDLGEVEFARIMTMVDPNAAGVV |
| | | TFQAFIDFMTRETAETDTAEQVVASFKILAGDKNYITAEELRRELPAEQAEYC |
| | | IRRMAPYKGAGAPAGALDYVAFSSALYGESDL |
| 111 | Pig | MAMSRPLTDQEKRKQISVRGLAGVENVAELKKNFNRHLHFTLVKDRNVAN |
| | A0A5G2QU59 | ARDYYFALAYTVRDHLVGRWIRTQQHYYEKDPKRIYYLSLEFYMGRTLQN |
| | (Alpha-1,4 | TMVNLALENACDEATYQLGLDMEELEEMEEDAGLGNGGLGRLAACFLDSM |
| | glucan | ATLGLAAYGYGIRYEFGIFNQKITGGWQMEEADDWLRYGNAWEKARPEFT |
| | phosphorylase) | LPVHFYGRVEHTNQGAKWVDTQVVLAMPYDTPVPGYRNNVVNTMRLWSA |
| | | KAPNDFNLKDFNVGGYIQAVLDRNLAENISRVLYPNDNFFEGKELRLKQEYF |
| | | VVAATLQDIIRRFKSSKFGCRDPVRTSFDAFPDKVAIQLNDTHPSLAIPELMRI |
| | | LVDEERLEWEKAWDVTVRTCAYTNHTVLPEALERWPVHLMETLLPRHLQII |
| | | YEINQRFLNRVAAAYPGDVDRLRRMSLVEEGAVKRINMAHLCIAGSHAVNG |
| | | VARIHSEILKKTIFKDFYELEPHKFQNKTNGITPRRWLVMCNPGLAEVIAERI |
| | | GEEYIADLDQLRKLLSYVDDEAFIRDVAKVKQENKLKFSAYLEREYKVHINP |
| | | NSLFDIQVKRIHEYKRQLLNCLHVITLYNRIKKEPNKFVVPRTVMIGGKAAP |
| | | GYHMAKMIIKLITAIGDVVNHDPVVGDRLRVIFLENYRVSLAEKVIPAADLS |
| | | EQISTAGTEASGTGNMKFMLNGALTIGTMDGANVEMAEEAGEENFFIFGMR |
| | | VEDVERLDQKGYNAQEYYDRIPELRHVIDQLSSGFFSPKQPDLFKDIVNMLM |
| | | HHDRFKVFADYEDYIKCQERVSALYKNPREWTRMVIRNIATSGKFSSDRTIA |
| | | QYAREIWGVEPSRQRLPAPDEKI |
| 112 | Pig | MPHQYPALTPEQKKELSDIAHRIVAPGKGILAADESTGSIAKRLQSIGTENTE |
| | A0A286ZYX8 | ENRRFYRQLLLTADDRVNPCIGGVILFHETLYQKADDGRPFPQVIKAKGGVV |
| | (Fructose- | GIKVDKGVVPLAGTNGETTTQGLDGLSERCAQYKKDGADFAKWRCVLKIG |
| | bisphosphate | EHTPSSLAIMENANVLARYASICQQVGLQNGIVPIVEPEILPDGDHDLKRCQY |
| | aldolase) | VTEKVLAAVYKALSDHHIYLEGTLLKPNMVTPGHACTQKYSHEEIAMATVT |
| | | ALRRTVPPAVPGITFLSGGQSEEEASINLNAINKCPLLKPWALTFSYGRALQA |
| | | SALKAWGGKKENLKAAQEEYVKRALANSLACQGKYTPSGKAGAAASESLF |
| | | ISNHAY |
| 113 | Pig | MAPKKDVKKPAAAAAPAPAPAPAPAPAPAPPKEEKIDLSAIKSFSADQIAEFK |
| | A0A287BJF1 | EAFLLFDRTGECKITLSQVGDVLRALGTNPTNAEVKKVLGNPSNEEMNAKKI |
| | (Myosin light | EFEQFLPMLQAISNNKDQGSYEDFVEGLRVFDKEGNGTVMGAELRHVLATL |
| | chain 1) | GEKMKEEEVEALMAGQEDSNGCINYEAFVKHIMSI |
| 114 | Pig G3P | MVKVGVNGFGRIGRLVTRAAFNSGKVDIVAINDPFIDLHYMVYMFQYDSTH |
| | (Glyceraldehyde- | GKFHGTVKAENGKLVINGKAITIFQERDPANIKWGDAGATYVVESTGVFTT |
| | 3-phosphate | MEKAGAHLKGGAKRVIISAPSADAPMFVMGVNHEKYDNSLKIVSNASCTTN |
| | dehydrogenase) | CLAPLAKVIHDHFGIVEGLMTTVHAITATQKTVDGPSGKLWRDGRGAAQNII |
| | | PASTGAAKAVGKVIPELNGKLTGMAFRVPTPNVSVVDLTCRLEKPAKYDDI |
| | | KKVVKQASEGPLKGILGYTEDQVVSCDFNSDTHSSTFDAGAGIALNDHFVK |
| | | LISWYDNEFGYSNRVVDLMVHMASKE |
| 115 | Pig TPM1 | MDAIKKKMQMLKLDKENALDRAEQAEADKKAAEDRSKRLEDELVSLQKK |
| | (Tropomyosin | LKATEDELDKYSEAPKDAQEKLELAEKKATDAEADVASLNRRIQLVEEELD |
| | alpha-1 chain) | RAQERLATALQKLEEAEKAADESERGMKVIESRAQKDEEKMEIQEIQLKEA |
| | | KHIAEDADRKYEEVARKLVIIESDLERAEERAELSEGKCAELEEELKTVINNL |
| | | KSLEAQAEKYSQKEDKYEEEIKVLSDKLKEAETRAEFAERSVTKLEKSIDDL |
| | | EDELYAQKLKYKAISEELDHALNDMTSI |
| 116 | Pig | MPEAKPATKKAPAGKDAAKPAPKEAPPKGAPEEPPKEAPPEDQSPTAEEPTG |
| | A0A5G2QB13 | IFLKKPDSVSVENGKDALIVAKVNGKELPGKPEIKWFKGKWLELGSKSGARF |
| | (Myosin binding | SFKESHDSASNVYTLELRIGKVVLGDRGDYRLEVKAKDFCDSCAFNIDVEAP |
| | protein C2) | RQDSAGQSLESFKRSGEGKPEGAGELDFSGLLKKRQVVEEEKKKKKDDDDL |
| | | GIPPEIWELLKGAKKSEYEKIAFQYGITDLRGMLKRLKKAKVEVKKSAAFTK |
| | | KLDPAYQVDQGNKIKLVVEISDPDLPLKWYKNGQEIKPSSKYVFENVGKKRI |
| | | LCINKCTLADDAAYEVAVKDEKCFTELFVREPPVLIVTPLEDQQVYVGDRVE |
| | | MTVEVSEEGAQVMWLKDGVELTREDSFKSRYRFKKDGKRHILIYSDVSLED |
| | | RGRYQVMTNGGQCEAELSVEEKQLEVLQDIADLTVKASEQAVFKCEVSDEK |
| | | VTGKWYKNGVEVRPSKRITISHVGRFHKLVIDDVRPEDEGDYTFVPDGYALS |
| | | LSAKLNFLEIKVEYVPKQEPPKIHLDCSGKTSENAVVVVAGNKLRLDVSITG |
| | | EPPPVATWMKEDEVFSGTEGRVRLEQRADISSFVIESAERGDEGRYTIKVTNP |
| | | AGEDVASIFLRVVDVPDPPEAVRVTTVGEDWAILVWEPPKYDGGQPVTGYL |
| | | LERKKKGSQRWMKLNFEVFTETTYESTNMIEGILYEMRVFAVNAIGVSQPSV |
| | | NTKPFMPIAPTSEPQHLTVEDVTDTTTTLKWRPPDRIGAGGIDGYLVEYCVE |
| | | GSDEWIPANTEPVERCGFTVKNLPTGARITFRVVGVNIAGRSQPATLAQPVTI |
| | | REIVGEPPRQSSVLIRASPDSLVATTLSVPLRRVPQPYAEPAPGIVSPEQPKIRL |
| | | PRHLRQTYIRKVGEHLNLVIPFQGKPRPQVVWTKGGAPIDTSRVHVRTSNFD |
| | | TVFFVRQAARSDSGEYELSVQIENMKDTATVHIRVVEKAGPPQNVMVKEV |
| | | WGTNALVEWQPPKDDGNTEITGYFVQKADKKTMEWFNVYEHNRHTSCTV |
| | | SDLIMGNEYYFRVYSENICGLSDSPGVSKNTARILKTGITFKLPEFKEHDFRTP |
| | | PKFLTPLPDRVVVAGYTAALNCAVRGHPKPKVVWMKNQMEIGEDPKFLMT |
| | | NHQGVLTLNIRRPSPFDSGTYSCRASTSWGRPWPSASWKSECRSEAPASTQG |
| | | SWLLSPDPDLPEPLSLSRVITRFLSRTQ |
| 117 | Pig | TMQHVYTKMSLVAVPFYQKRHRHFDQSYRNIQTRYLLDEYAAKKRASTQS |
| | A0A287BSB6 | SSQKSVTQESSSQRESSSERASSQTLLGGTTCRLCAKRVSTALEEEEQERKRR |
| | (Uncharacterized | YHSLLAAYGEAKRERYLSELAQLEEDVHLARTHARDKLDRFALQHVDTHA |
| | protein) | LRHAVGDRLAWERHSFEDRISRAPEILVRLRSHTVWERMSVKLCFTVQGFPT |
| | | PVVQWYKDGSLICQAGEPGKYRIESKYGVHTLEINRADFDDTATYSAVATN |
| | | VHGQVSTNAAVVVRRFHGDEEPFHSVGLPIRLPLSSVIPYTHFDVQFLEKFGV |
| | | TFRREGETLTLKCTLLVTPDLKRVQPRAEWYRDDVLLKESKWTKMFFGEGQ |
| | | ASLSFSHLNKDDEGLYTLRIVSRGGISDHSAFLFVRDADPLVMGAPGAPMDV |
| | | HCHDANRDYVIVTWKPPNTTKESPVLGYFIDRCEVGTDNWVQCNDAPVKIC |
| | | KYPVSGLVEGRSYTFRVRAVNSAGVSRPSRASEAVAALDPLDLKRLQGIHW |
| | | EGDKDIVIYQDDLEGEVQIPRPPTNVHASEISRTYVVLSWDPPTPRGKEPLMY |
| | | FIEKSVVGSGTWQRENAQTAVRSPRYAVFDLAEGKSYVFRVLSANKHGLSD |
| | | PSEITSPIQAQDTIVVPSAPGRVLASRNTKTSVVVQWDQPKHGEDLLGYYVD |
| | | CCVAGSNVWEPCNHKPIGYNRFVVHGLTTGEQYIFRVKAVNAVGTSENSQE |
| | | SDVIKVQAALTVPSHPYGITLLNCDGHSMTLGWKVPKFSGGSPIIGYYVDKR |
| | | EGHHKNWHEVNSSPLKERILTVEGLTEGSTYEFKIAAPTWWGSGCRRIPVSS |
| | | SSVRPGQRLSQVPTSGWVGTLGFFLVLLSLYRRYLLGSGEHGDS |
| 118 | Pig ENOB | MAMQKIFAREILDSRGNPTVEVDLHTAKGRFRAAVPSGASTGIYEALELRDG |
| | (Beta-enolase) | DKSRYLGKGVLKAVEHINKTLGPALLEKKLSVVDQEKVDKFMIELDGTENK |
| | | SKFGANAILGVSLAVCKAGAAEKGVPLYRHIADLAGNPDLVLPVPAFNVING |
| | | GSHAGNKLAMQEFMILPVGASSFREAMRIGAEVYHHLKGVIKGKYGKDAT |
| | | NVGDEGGFAPNILENNEALELLKTAIQAAGYPDKVVIGMDVAASEFYRNGK |
| | | YDLDFKSPDDPSRHITGEKLGELYKSFIKNYPVVSIEDPFDQDDWKTWTSFLS |
| | | GVNIQIVGDDLTVTNLKRIAQAVEKKACNCLLLKVNQIGSVTESIQACKLAQ |
| | | SNGWGVMVSHRSGETEDTFIADLVVGLCTGQIKTGAPCRSERLAKYNQLMR |
| | | IEEALGDKAVFAGRKFRNPKAK |
| 119 | Pig Q5XLD2 | MAPKKAKRRAAAEGSSNVFSMFDQTQIQEFKEAFTVIDQNRDGIIDKEDLRD |
| | (Myosin light | TFAAMGRLNVKNEELDAMMKEASGPINFTVFLTMFGEKLKGADPEDVITGA |
| | chain) | FKVLDPEGKGTIKKHFLEELLTTQCDRFSQEEIKNMWAAFPPDVGGNVDYK |
| | | NICYVITHGDAKDQE |
| 120 | Pig MYH1 | MSSDQEMAIFGEAAPYLRKSEKERIEAQNKPFDAKTSVFVAEPKESFVKGTV |
| | (Myosin-1) | QSREGGKVTVKTEAGATLTVKEDQVFPMNPPKFDKIEDMAMMTHLHEPAV |
| | | LYNLKERYAAWMIYTYSGLFCVTVNPYKWLPVYNAEVVTAYRGKKRQEAP |
| | | PHIFSISDNAYQFMLTDRENQSILITGESGAGKTVNTKRVIQYFATIAVTGEKK |
| | | KEEPTSGKMQGTLEDQIISANPLLEAFGNAKTVRNDNSSRFGKFIRIHFGTTG |
| | | KLASADIETYLLEKSRVTFQLKAERSYHIFYQIMSNKKPELIEMLLITTNPYDY |
| | | AFVSQGEITVPSIDDQEELMATDSAIEILGFTSDERVSIYKLTGAVMHYGNLK |
| | | FKQKQREEQAEPDGTEVADKAAYLQGLNSADLLKALCYPRVKVGNEFVTK |
| | | GQTVQQVYNAVGALAKAVYDKMFLWMVTRINQQLDTKQPRQYFIGVLDIA |
| | | GFEIFDFNSLEQLCINFTNEKLQQFFNHHMFVLEQEEYKKEGIEWEFIDFGMD |
| | | LAACIELIEKPMGIFSILEEECMFPKATDTSFKNKLYEQHLGKSNNFQKPKPA |
| | | KGKVEAHFSLIHYAGTVDYNITGWLDKNKDPLNETVVGLYQKSSVKTLAFL |
| | | FTGAAGADAEAGGGKKGGKKKGSSFQTVSALFRENLNKLMTNLRSTHPHF |
| | | VRCIIPNETKTPGAMEHELVLHQLRCNGVLEGIRICRKGFPSRILYADFKQRY |
| | | KVLNASAIPEGQFIDSKKASEKLLGSIDIDHTQYKFGHTKVFFKAGLLGLLEE |
| | | MRDEKLAQLITRTQARCRGFLARVEYQKMVERRESIFCIQYNIRAFMNVKH |
| | | WPWMKLYFKIKPLLKSAETEKEMANMKEEFEKTKESLAKAEAKRKELEEK |
| | | MVALMQEKNDLQLQVQAEADSLADAEERCDQLIKTKIQLEAKIKEVTERAE |
| | | DEEEINAELTAKKRKLEDECSELKKDIDDLELTLAKVEKEKHATENKVKNLT |
| | | EEMAGLDETIAKLTKEKKALQEAHQQTLDDLQAEEDKVNTLTKAKTKLEQ |
| | | QVDDLEGSLEQEKKLRMDLERAKRKLEGDLKLAQESTMDIENDKQQLDEK |
| | | LKKKEFEMSNLQSKIEDEQALAMQLQKKIKELQARIEELEEEIEAERASRAK |
| | | AEKQRSDLSRELEEISERLEEAGGATSAQIEMNKKREAEFQKMRRDLEEATL |
| | | QHEATAATLRKKHADSVAELGEQIDNLQRVKQKLEKEKSEMKMEIDDLAS |
| | | NMETVSKAKGNLEKMCRTLEDQLSELKTKEEEQQRLINDLTAQRARLQTES |
| | | GEYSRQLDEKDTLVSQLSRGKQAFTQQIEELKRQLEEEIKAKSALAHAVQSS |
| | | RHDCDLLREQYEEEQEAKAELQRAMSKANSEVAQWRTKYETDAIQRTEELE |
| | | EAKKKLAQRLQDAEEHVEAVNAKCASLEKTKQRLQNEVEDLMIDVERSNA |
| | | ACAALDKKQRNFDKILAEWKQKYEETHAELEASQKESRSLSTELFKVKNAY |
| | | EESLDQLETLKRENKNLQQEISDLTEQIAEGGKRIHELEKIKKQVEQEKSEIQ |
| | | AALEEAEASLEHEEGKILRIQLELNQVKSEVDRKIAEKDEEIDQLKRNHVRV |
| | | VESMQSMLDAEIRSRNDAIRLKKKMEGDLNEMEIQLNHANRMAAEALRNY |
| | | RNTQGILKDTQIHLDDALRSQEDLKEQLAMVERRANLLQAEIEELRATLEQT |
| | | ERSRKVAEQELLDASERVQLLHTQNTSLINTKKKLETDISQIQGEMEDIIQEA |
| | | RNAEEKAKKAITDAAMMAEELKKEQDTSAHLERMKKNLEQTVKDLQHRL |
| | | DEAEQLALKGGKKQIQKLEARVRELEGEVESEQKRNVETVKGLRKHERRVK |
| | | ELTYQTEEDRKNILRLQDLVDKLQAKVKSYKRQAEEAEEQSNVNLSKFRKL |
| | | QHELEEAEERADIAESQVNKLRVKSREVHTKIISEE |
| 121 | Pig F1SHX0 | MADDEEYEEVVEYYTEETVYEEVPGETITEVYETTTTRTSGYEQSDTSQPAV |
| | (Nebulin) | AQPMPSKPVERKKVIRKKVDPSKFMTPYIAHSQKMQDLFSTNKYKKNYEKA |
| | | KGKPYAITTDTPELRRIKKVQDQLSEVKYRIDGDVAKTICHVDEKAKDIEHA |
| | | KKVSQQVSKVLYKQNWEDTKDKYLLPPDAPELVQAIKNTAMFSKKLYTED |
| | | WEADKSLFYPYNDSPELRRVAQAQKALSDIAYKKGHAEQQTQFTSLPDPPDI |
| | | EFAKKVTNQVSKQKYKEDYENKIKGKWSETPCFEIATARMNAKNISTRKYQ |
| | | EDFENMKDQIYFMQTETPEYKVNKQAGTAASKVKYKEDYEKNKGKADYN |
| | | VLPASENPLLRHLKAAGDALSDKRYKENYEKTKAKSINYCETPKFKLDTVL |
| | | HNFSSDTKYKDSYLKNILGHYVGSFEDPYHTHCMKVAAENSDKNYKAEYE |
| | | EDRGKGFFPQTITQEYEAIKKLDQCKDHTYKVHPDKTKFTQVTDSPVLVQA |
| | | QVNSKQLSDLNYKAKHENEKFKCHIPPDTPAFIQHKVNAYNLSDNVYKHD |
| | | WEKTKAKKFDIKVDAIPLLAAKANTKNTSDVMYKKDYEKSKGKMIGALSIN |
| | | DDPKMLHSLKTAKNQSDRLYKENYEKTKAKSMNYCETPKYQLDTLLKNFS |
| | | EAKYKDSYVQNVLGHYIGSFEDPYQLHCMKVSAQNSDKNYKAEYEEDKGK |
| | | CYFPQTITQEYEAIKKLDQCKDHTYKVHPDKTKFTAVTDTPVLLQAQLNTK |
| | | QLSDLNYKAKHEGEKFKCNIPADAPQFIQHRVNAYNLSDNVYKHDWEKTK |
| | | AKKFDIKVDAIPLLAAKANTKNTSDVMYKKDYEKSKGKMIGALSINDDPKM |
| | | LHSLKTAKNQSDREYRKDYEKSKTIYTAPLDMLQVTQAKKSQAIASDVDYK |
| | | HLLHNYSYPPDSINVDLAKKAYALQSDVEYKADYNSWMKGCGWIPFGSLE |
| | | MEKAKRASDILNEKKYRQHPETLKFTSIEDAPIIVQSKINQAQRSDIAYKAKG |
| | | EEIIHKYSLPADLPFIQAKVNAYNISESMYKADLKDLSKKGYDLRTDAIPIKA |
| | | AKAARQAASDVQYKKDYEKAKGKMVGFQSLQDDPKLVHYMNVAKIQSDR |
| | | EYKKDYEKTKTKYNTPHDMFNVVAAKKAQDVASNVNYRHSLHHYTYLPD |
| | | AMDLELSKNMMHIQSDNAYKEDYNTWMKGIGWIPLGSLEVEKVKKAGDA |
| | | LNEKKYRQHPDTLKFTSIVDSPVMVQAKQNTRQVSDILYKAKGEDVKHKYT |
| | | MGPDLPQFLQAKCNAYNISDVCYKRDWHDLIAKGNNVLGDAIPITAAKASR |
| | | NIASDYKYKEAYEKAKGKHVGFRSLQDDPKLVHYMNVAKLQSDREYKKN |
| | | YENTKTSYHTPGDMVSITAAKMAQDVATNVNYKQPIHHYTYLPDALSLEHT |
| | | RNVNQIQSDNVYKDEYNHFFKGIGWIPLGSLEVEKVKKAGDALNERKYRQH |
| | | PDTIKFTSVPDSMGMVLAQHNTKQLSDLNYKVEGEKLKHKYTMDPDVPQFI |
| | | QAKVNAINMSDAHYKADWKKTLAKGYDLTPDAIPIVAAKSSRNIASDFKYK |
| | | EAYEKAKGKQVGFRSLQDDPKLVHYMNVAKIQSDREYKKGYEASKTRYHT |
| | | PLDMLSVTAAKKSQEVATNTNYKQPFHHYTLLPDALNVEHSRNAMQIQSDN |
| | | LYKSDFTNWMKGIGWIPMESLEVEKAKKAGEILSEKKYRQHPEKLKFTYAM |
| | | DTMEQALNKSNKLIMDKRLYTEKWNKDKTTIHVMPDTPDILLSRVNQITLS |
| | | DKLYKAGWEEEKKKGYDLRPDAISIKAARASRDIASDYKYKQAYEQAKGK |
| | | HIGFRSLEDDPKLVHFMQVAKMQSDREYKKAYEKSKTSFHTPVDMLSVVA |
| | | AKKSQEVATNANYRNVIHTYNMLPDAMSFELAKNMMQLQSDNQYKADYA |
| | | DFMKGIGWLPLGSLEAEKNKKAMEIISEKKYRQHPDTLKYSTLMDSMNMVL |
| | | AKNNAKIMNEHLYKQAWEADKTKVHIMPDIPQIILAKANAVNMSDKLYKLS |
| | | LEESKKKGYDLRTDAISIQAAKASRDIASDYKYKHSYEKERGKMVGFRSLED |
| | | DPKLVHSMQVAKMQSDREYKKSYEKTKTSYHTPADMLSVTAARDAQANIT |
| | | NTNYKHLIHKYILLPDAMNIQLSKNMNHIQSDNEYKQDYNECYKGLGWSPA |
| | | GSLEVEKAKKATEYASDQKYRQHPSNFQFQKLNDSMDMVLAKQNAHTMN |
| | | KYLYTADWNKDKTKIHVMPDTPDILQAKQNQTLFSQKLYKLGWEEALKKG |
| | | YDLPVDAISVQLAKVSRDNASDFKYKQGYRKQLGHHIGFRNLQDDPKLVLS |
| | | MNVAKMQSEREYKKDFEKWKTKFTSPVDMLGVVLAKKCQALVSDVDYKN |
| | | YLHQWTCLPDQNDVIQAKRSYELQSENLYKSDLEWLRGIGWSPLGSLEAEK |
| | | NKRASEIISEKKYRQPPDRNKFTSIPDAMDIVLAKTNAKNRSDRLYREAWDK |
| | | DKTQVHIMPDTPDIILAKANLINSSDKLYRMGYEELKKKGYDLPVDAIPIKA |
| | | AKASREIASEYKYKEGFRKQLGHHVGARNIEDDPKMMWSMHVAKIQSDRE |
| | | YKKDFEKWKTKFSSPVDMLGVVLAKKCQTLVSDIDYKNYLHQWTCLPDQS |
| | | DVIHARRAYDLQSDNLYKSDLQWLKGIGWLPSGSLEDEKNKRATQILSDHV |
| | | YRQHPDKFKFSSLMDSIPMVLAKNNAITMNHRLYTEAWDKDKTTVHIMPDT |
| | | PEVLLAKQNKINYSEKLYKLGLEEAKRKGYDMRLDAIPIRAAKASRDIASEF |
| | | KYKEGYRKQLGHHIGARAIHDDPKMMWSMHVAKVQSDREYKKDFEKWKT |
| | | KFSSPVDMLGVVLAKKCQTLVSDIDYKNYLHQWTCLPDQSDVIHARRAYDL |
| | | QSDNMYKSDLQWLRGIGWVPIGSLDVEKCKRATEILSDKIYRQPPDTFKFTS |
| | | VPDSLEQVLAKNNAINMNKRLYTEAWDKDKTQVHIMPDTPEIMLARQNRL |
| | | NYSETLYKLANEEAKKKGYDLRSDAIPIVAAKASRDIVSDYKYKDGYRKQL |
| | | GHHIGARNIEDDPKMMWSMHVAKIQSDREYKKDFEKWKTKFSSPVDMLGL |
| | | VQAKKCQILVSDVDYKNYLHQWTCLPDQNDVIHARQAYDLQSDNIYKSDL |
| | | QWLKGIGWVPIGSVDVVKCKRAAEILSDHIYRQPPDKLKFTSVPDSLEQVLA |
| | | KNNAINMNKRLYTEAWDKDKTQIHIMPDTPEIMLARQNKINYSESLYRQAM |
| | | EEAKKEGYDLRSDAIPIVAAKASRDIASDYKYKEAYRKQLGHHIGARAVHD |
| | | DPKMMWSLHVAKMQSDREYKKDFEKYKTRFSSPVDMLGIVLAKKCQTLVS |
| | | DVDYKHPLHEWTCLPDQNDIIHARKAYDLQSDNLYKSDLEWMKGIGWIPLG |
| | | SVEVVKAKRAGEILSDNIYRQRPDTLKFTSVTDSPELVLAKNNALNMNKRL |
| | | YTEAWDNDKKTIHVMPDTPEIMLAKLNRINYSDKLYRLALEESKKEGCDLR |
| | | VDAIPIQAAKASREIASDYKYKEAYRKQLGHHIGARNIEDDPKMMWSIHAA |
| | | KIQSDREYKKDFEKWKTRFSSPVDMLGLVQAKKCQILVSDIDYKHPLHEWIC |
| | | LPDQNDIIQARKAYDLQSDAIYKADLEWLRGIGWVPLGSVEVEKVKRAGEIL |
| | | SERKYRQPADQLKFTCITDTPEIVLAKNNALTMSKHLYTEAWDTDKTSIHV |
| | | MPDTPEILLAKSNSANISQKLYTKGWDESKMKDYDLKADAISIKSAKASRDI |
| | | ASDYKYKEAYEKQKGHHIGAQSVEDDPKIMCAIHAGKIQSEREYKKDFQKW |
| | | KTKFTSPVDMLGILLAKKCQTLVSDIDYRNYLHHWTCLPDQNDIIQARKAYE |
| | | LQSDALYKADLEWLRGIGWMPQGSPEVLRVKNAQEILRDSVYRTPVVKLK |
| | | YTSIVDTPEVVLAKSNAENISIPKYREVWDKDKTSVHIMPDTPEINLARANAL |
| | | NVSNKIYREGWDEMKMSCDVRLDAIPIQAAKASREIASDYKYKLDHEKQKG |
| | | HYVGTLTARDDNKIRWALIAGKIQNEREYRLHWAKWKSKFQSPADMLSITH |
| | | SKNCQALVSDIDYRNYLHQWTCLPDQHDVIQARKAYDLQSDAIYKADLEW |
| | | LRGIGWMPSDSVSVNHAKHAADIFSEKKYRTKIETLNFTPVDDRVDYVTAK |
| | | QSGEILNDIKYRKDWNDTKSKYTLTETPQLHAAQEAARILDQYLYKESWEK |
| | | QKATGYILPPDAVPFVHAHHSSDVQSELKYKADHVKQKGHYVGVPTMRDD |
| | | PKLVWFEHAGQIQNDRLYKEHYHKTKAKINIPPDMVSVVAAKEGQILVSDID |
| | | YRNYLHQWTCHPDQNDVIQARKAYDLQSDNVYKADLEWLRGIGWIPLDSV |
| | | DHVRVTRNQEMVNQIKYKKDALANYPNFTSVVDPPAVVLAKINSVNQSDV |
| | | KYKETFNKLLKGKYIFSPDTPYITHSKDMGKLYSTILYKGAWEGTKAYGYTL |
| | | DERYIPIVGAKHADFVNSELKYKETYEKQKGHYLAGKEISEFPNVVHCLDFQ |
| | | KMRSVLNYRRHYEDTKANVHIPNDMMNHVLAKRCQYILSDLEYRHYFHQ |
| | | WTSLPEEPNVIRARNAQEILSDNVYKDDLNWLKGIGCYVWDTPQILHAKKS |
| | | YDLQSQILYTADGKENLKNYNMVTDTPLYVTALQSGINASEVKYKENYHQI |
| | | KDKYTTVLETVDYDRTRNLKDLYSSNVYKEAWDKVKATSYILPSNTVSLTH |
| | | AKNQKHLASHIKYREEYEKFKALYTLPKSVDDDPNTARCLRVGKFNIDRLY |
| | | RSVYEKNKMKIHMVPDMVEMVTAKDSQKKVSEIDYRLHLHEWICHPDLQV |
| | | NSHVRKVTDQISDIVYKDDLNWLKGIGCYVWDTPEILHAKHAYDLRNDIKY |
| | | QAHVKKTRNNYKLVTDTPLYVQAVKSGKQLSDAVYHYDYVHSVRGKVAP |
| | | TTKTVDLDRALHAYKLQSENLYRKDGLNSLPTGYRLPGDTPHFKHNKDIRY |
| | | MSSYFKYKEVYEHFKANGYTLGPKDVPFVHVRRVNNVTSERLYRQLYHKL |
| | | KDKIHTTPDTPEIRQVKKTQEAVSELIYKSDFFKMQGHMISMPYTPHVLHCR |
| | | YVGDITSDIKYKEDLQILRGLGCFLYDTPDMVRSRHLRKLWSNYLYTDNAR |
| | | KMRDKYKVVLDTPEYRKVQELKTHLSELVYRAAGKKQKSIFTSVPDTPDLL |
| | | RAKRGQKLQSQYLYVELATKERPHHHAGNQTTALKHARDVKDMVSEKKY |
| | | KVQYEKMKDKYTPVPDTPILIRAKKAYWNASDLRYKETFQKTKGKYHTVK |
| | | DALDIVYHRKVTDHISKIKYKENYMSQLGIWRSIPDRPEHFHHRAVTDAVSD |
| | | VKYKEDLTWLKGIGCYAYDTPDFTLAEKNKTLYSKYKYKEVFERTKSDFKY |
| | | EADCPINRHFKFATQLMNEKKYRADYEQRKDKYQLVVDEPRHLLAKIAGD |
| | | QISQIKYRKNYEKTKDKFTSVVDTPEHLRTTKVNKQISDILYKLEYNKAKPR |
| | | GYTTIHDTPMLLHVRKVKDEVSDLKYKEVYQRTKSNCTIEPDAVHIKAAKD |
| | | AYKVNTNLDYKKQYEANKAHWRWTPDRPDFIQAAKSSLQQSDFEYKLDRE |
| | | FLKGCKLSVTDDKDMVLALRNSVIESDLKYKEKHVKERGTCHAVPDTPQIL |
| | | LAKTVSNLVSENKYKRHVKKHLAQGSYTTLPETRDTVHVKEVTKHVSDTN |
| | | YKKKFVKERGKSNYSIMLEPPEVKHAMEVAKKQSDVAYKKDAKENLHYTT |
| | | VADRPDIKKATQAAKQASEVEYRAKHRKEGSHGLSMLGRPDIEMAKKAAT |
| | | LSSQVKYRENFNKEKGKTPKYNPKESQLYKIMKDANNLASEVKYKADLKK |
| | | LHKPVTDMKESLIMNHVLNTSHLASSYQYKKNYEKSKGHYHTIPDNLEQLH |
| | | LKEATELQSIVKYKEKYEKERGKPMLDFETPTYITAKESQQMQSGKEYRKD |
| | | YEESIKGRNLTGLEVTPALLHVKYATKIASEKEYRKDLEESIRGKGLSEMEDT |
| | | PDMLRAKNATQILNEKEYKRDLELEVKGRGLNAMANETPDFMRARNATDI |
| | | ASQIKYKQLAEMEKANFTSVVDTPEIIHAQQVKNLSSQKKYKEDAEKCMSY |
| | | YETVLDTPEMQRVRENQKNFSLLQYQCDLKNSKGKITVVQDTPEILRVKEN |
| | | QKNFSSVLYKEDVSPGTAIGKTPEMMRVKQTQDHISSVKYKETIGQGTPIPD |
| | | LPEVKRVKETQKHISSVMYKENLGTGIPTPVTPEIERVKRNQENFSSVLYKEN |
| | | LGKGTPTPITPEMERVKRNQENFSSVLYKENMGKGTPLPVTPEMERVKHNQ |
| | | ENISSILYKENVGKGTPTPVTPEMQRVKRNQENISSVIYKENLGKATPTPFTPE |
| | | MERVKRNQENFSSVLYKENMRKATPTPVTPEMERAKRNQENISSVLYSDSF |
| | | RKQIQGKAAYVLDTPEMRRVRETQRHISTVKYHEDFEKHKGCFTPVVTDPIT |
| | | ERVKKNTQDFSDINYRGIQRKVVEMEQKRNDQDQETITGLRVWRTNPGSVF |
| | | DYDPAEDNIQSRSLHMLNAQAQRRSREQSRSASALSISGGEEKSEHSEAARL |
| | | STYSDGGVFFSATSAAYKHAKTTELPQQRSSSVATQQTTVSSIPSHPSTAGKI |
| | | FRAMYDYMAADADEVSFRDGDAIVNVQAIDEGWMYGTVQRTGRTGMLPA |
| | | NYVEAI |
| 122 | Pig MYH4 | MSSDQEMAIFGEAAPYLRKSEKERIEAQNKPFDAKTSVFVAEPKESFVKGTV |
| | (Myosin-4) | QSREGGKVTVKTEAGATLTVKEDQVFPMNPPKFDKIEDMAMMTHLHEPAV |
| | | LYNLKERYAAWMIYTYSGLFCVTVNPYKWLPVYNAEVVTAYRGKKRQEAP |
| | | PHIFSISDNAYQFMLTDRENQSILITGESGAGKTVNTKRVIQYFATIAVTGEKK |
| | | KEEPTPGKMQGTLEDQIISANPLLEAFGNAKTVRNDNSSRFGKFIRIHFGTTG |
| | | KLASADIETYLLEKSRVTFQLKAERSYHIFYQIMSNKKPELIEMLLITTNPYDY |
| | | AFVSQGEITVPSIDDQEELMATDSAIEILGFTSDERVSIYKLTGAVMHYGNLK |
| | | FKQKQREEQAEPDGTEVADKAAYLQGLNSADLLKALCYPRVKVGNEFVTK |
| | | GQTVQQVYNAVGALAKAVYDKMFLWMVTRINQQLDTKQPRQYFIGVLDIA |
| | | GFEIFDFNSLEQLCINFTNEKLQQFFNHHMFVLEQEEYKKEGIEWEFIDFGMD |
| | | LAACIELIEKPMGIFSILEEECMFPKATDTSFKNKLYEQHLGKSNNFQKPKPA |
| | | KGKAEAHFSLIHYAGTVDYNITGWLDKNKDPLNETVVGLYQKSSVKTLAFL |
| | | FAERQSSEEGGTKKGGKKKGSSFQTVSALFRENLNKLMTNLRSTHPHFVRCI |
| | | IPNETKTPGAMEHELVLHQLRCNGVLEGIRICRKGFPSRILYADFKQRYKVLN |
| | | ASAIPEGQFIDSKKASEKLLGSIDIDHTQYKFGHTKVFFKAGLLGTLEEMRDE |
| | | KLAQLITRTQAMCRGFLMRVEFRKMMERRESIFCIQYNIRAFMNVKHWPW |
| | | MKLYFKIKPLLKSAETEKEMANMKEEFEKTKEDLAKSEAKRKELEEKMVAL |
| | | MQEKNDLQLQVQAEADGLADAEERCDQLIKTKIQLEAKIKEVTERAEDEEEI |
| | | NAELTAKKRKLEDECSELKKDIDDLELTLAKVEKEKHATENKVKNLTEEMA |
| | | GLDENIAKLTKEKKALQEAHQQTLDDLQAEEDKVNTLTKAKTKLEQQVDD |
| | | LEGSLEQEKKLRMDLERAKRKLEGDLKLAQESTMDIENDKQQLDEKLKKKE |
| | | FEMSNLQSKIEDEQALAMQLQKKIKELQARTEELEEEIEAERASRAKAEKQR |
| | | SDLSRELEEISERLEEAGGATSAQIEMNKKREAEFQKMRRDLEEATLQHEAT |
| | | AAALRKKHADSVAELGEQIDNLQRVKQKLEKEKSELKMEIDDLASNMETVS |
| | | KAKGNLEKMCRTLEDQLSEVKTKEEEHQRLINELSAQKARLQTESGEFSRQL |
| | | DEKEALVSQLSRGKQAFTQQIEELKRQLEEETKAKSALAHAVQSSRHDCDLL |
| | | REQYEEEQEAKAELQRAMSKANSEVAQWRTKYETDAIQRTEELEEAKKKL |
| | | AQRLQDAEEHVEAVNAKCASLEKTKQRLQNEVEDLMLDVERSNAACAALD |
| | | KKQRNFDKILAEWKHKYEETQAELEASQKESRSLSTELFKVKNAYEESLDQ |
| | | LETLKRENKNLQQEISDLTEQIAEGGKHIHELEKVKKQIEQEKSELQAALEEA |
| | | EASLEHEEGKILRIQLELNQVKSEIDRKIAEKDEEIDQMKRNHIRVVESMQST |
| | | LDAEIRSRNDALRIKKKMEGDLNEMEIQLNHANRQATEAIRNLRNTQGVLK |
| | | DTQLHLDDAIRGQDDLKEQLAMVERRANLMQAEIEELRASLEQTERSRRVA |
| | | EQELLDASERVQLLHTQNTSLINTKKKLETDISQIQGEMEDIVQEARNAEEKA |
| | | KKAITDAAMMAEELKKEQDTSAHLERMKKNMEQTVKDLQHRLDEAEQLA |
| | | LKGGKKQIQKLEARVRELENEVENEQKRNVEAVKGLRKHERRVKELTYQT |
| | | EEDRKNVLRLQDLVDKLQSKVKAYKRQAEEAEEQSNVNLSKFRKLQHELEE |
| | | AEERADIAESQVNKLRVKSREVHTKVISEE |
| 123 | Pig ALBU | MKWVTFISLLFLFSSAYSRGVFRRDTYKSEIAHRFKDLGEQYFKGLVLIAFSQ |
| | (Albumin) | HLQQCPYEEHVKLVREVTEFAKTCVADESAENCDKSIHTLFGDKLCAIPSLR |
| | | EHYGDLADCCEKEEPERNECFLQHKNDNPDIPKLKPDPVALCADFQEDEQK |
| | | FWGKYLYEIARRHPYFYAPELLYYAIIYKDVFSECCQAADKAACLLPKIEHL |
| | | REKVLTSAAKQRLKCASIQKFGERAFKAWSLARLSQRFPKADFTEISKIVTDL |
| | | AKVHKECCHGDLLECADDRADLAKYICENQDTISTKLKECCDKPLLEKSHCI |
| | | AEAKRDELPADLNPLEHDFVEDKEVCKNYKEAKHVFLGTFLYEYSRRHPDY |
| | | SVSLLLRIAKIYEATLEDCCAKEDPPACYATVFDKFQPLVDEPKNLIKQNCEL |
| | | FEKLGEYGFQNALIVRYTKKVPQVSTPTLVEVARKLGLVGSRCCKRPEEERL |
| | | SCAEDYLSLVLNRLCVLHEKTPVSEKVTKCCTESLVNRRPCFSALTPDETYK |
| | | PKEFVEGTFTFHADLCTLPEDEKQIKKQTALVELLKHKPHATEEQLRTVLGN |
| | | FAAFVQKCCAAPDHEACFAVEGPKFVIEIRGILA |

An animal protein can comprise a bison protein (Table 6). A bison protein can comprise a bison muscle protein. A bison protein can comprise a bison blood protein. A bison protein can comprise a bison albumin protein. A bison protein can comprise a sequence that has at least or about 70%, at least or about 75%, at least or about 80%, at least or about 90%, at least or about 91%, at least or about 92%, at least or about 93%, at least or about 94%, at least or about 95%, at least or about 96%, at least or about 97% at least or about 98% at least or about 99% or about 100% sequence identity to any one of SEQ ID NO: 124-138.

A food product may comprise a fragment or a truncation of a bison protein. The food product may include a fragment or truncation of any one of SEQ ID Nos: 124-138. A sequence of the fragment or truncation may be identical or substantially identical to a portion of any one of SEQ ID Nos: 124-138. A sequence of the fragment or truncation may be at least about 70%, 80%, 90%, 95%, 98%, or more identical to a portion of a sequence of any one of SEQ ID Nos: 124-138. A length of the fragment or truncation may be at least about 10%, 15%, 20%, 30%, 40%, 50%, 60%, 70%, 80%, 90%, or more of a length of any one of SEQ ID Nos: 124-138. A length of the fragment or truncation may be less than or equal to about 90%, 80%, 70%, 60%, 50%, 40%, 30%, 20%, 15%, 10%, or less than a length of any one of SEQ ID Nos: 124-138.

TABLE6
Bison Proteins

| SEQ | | |
|---|---|---|
| ID NO | Description | Amino Acid Sequence |
| 124 | Bison | MPFGNTHNKHKLNFKAEEEYPDLSKHNNHMAKALTLEIYKKLRDKETPSG |
| | A0A6P3IIV2 | FTLDDVIQTGVDNPGHPFIMTVGCVAGDEESYTVFKDLFDPIIQDRHGGFKP |
| | (Creatine kinase) | TDKHKTDLNHENLKGGDDLDPNYVLSSRVRTGRSIKGYALPPHCSRGERR |
| | | AVEKLSVEALNSLTGEFKGKYYPLKSMTEQEQQQLIDDHFLFDKPVSPLLL |
| | | ASGMARDWPDARGIWHNDNKSFLVWVNEEDHLRVISMEKGGNMKEVFR |
| | | RFCVGLQKIEEIFKKAGHPFMWNEHLGYVLTCPSNLGTGLRGGVHVKLAH |
| | | LSKHPKFEEILTRLRLQKRGTGGVDTAAVGSVFDVSNADRLGSSEVEQVQL |
| | | VVDGVKLMVEMEKKLEKGQSIDDMIPAQK |
| 125 | Bison | MNQIEPGVQYNYVYEDDEYMIQEEEWDRDLLLDPAWEKQQRKTFTAWCN |
| | A0A6P3J4W6 | SHLRKAGTQIENIEEDFRNGLKLMLLLEVISGERLPKPDRGKMRFHKIANVN |
| | (Alpha-actinin-2) | KALDYIASKGVKLVSIGAEEIVDGNVKMTLGMIWTIILRFAIQDISVEETSAK |
| | | EGLLLWCQRKTAPYRNVNIQNFHTSWKDGLGLCALIHRHRPDLIDYSKLNK |
| | | DDPIGNINLAMEIAEKHLDIPKMLDAEDIVNTPKPDERAIMTYVSCFYHAFA |
| | | GAEQAETAANRICKVLAVNQENERLMEEYERLASELLEWIRRTIPWLENRT |
| | | PEKTMQAMQKKLEDFRDYRRKHKPPKVQEKCQLEINFNTLQTKLRISNRPA |
| | | FMPSEGKMVSDIAGAWQRLEQAEKGYEEWLLNEIRRLERVEHLAEKFRQK |
| | | ASTHETWAYGKEQILLQKDYESSTLTEVRALLRKHEAFESDLAAHQDRVE |
| | | QIAAIAQELNELDYHDAVNVNDRCQKICDQWDRLGTLTQKRREALERTEK |
| | | LLETIDQLHLEFAKRAAPFNNWMEGAMEDLQDMFIVHSIEEIQSLITAHEQF |
| | | KATLPEADGERQSILAIQNEVEKVIQSYSIRISSSNPYSTVTVDEIRSKWDKV |
| | | KQLVPIRDQSLQEELARQHANERLRRQFAAQANAIGPWIQNKMEEIARSSIQ |
| | | ITGALEDQMNQLKQYEHNIINYKNNIDKLEGDHQLIQEALVFDNKHTNYTM |
| | | EHIRVGWELLLTTIARTINEVETQILTRDAKGITQEQMNEFRASFNHFDRRK |
| | | NGLMDHEDFRACLISMGYDLGEAEFARIMTLVDPNGQGTVTFQSFIDFMTR |
| | | ETADTDTAEQVIASFRILASDKPYILAEELRRELPPDQAQYCIKRMPAYSGP |
| | | GSVPGALDYTAFSSALYGESDL |
| 126 | Bison | MSRPLTDQEKRKQISVRGLAGVENVTELKKNFNRHLHFTLVKDRNVATPR |
| | A0A6P3HAI4 | DYYFALAYTVRDHLVGRWIRTQQHYYEKDPKRIYYLSLEFYIGRTLQNTM |
| | (Alpha-1,4 glucan | VNLALENACDEATYQLGLDMEELEEIEEDAGLGNGGLGRLAACFLDSMAT |
| | phosphorylase) | LGLAAYGYGIRYEFGIFNQKISGGWQMEEADDWLRYGNPWEKARPEFTLP |
| | | VHFYGRVEHTSQGAKWVDTQVVLAMPYDTPVPGYRNNVVNTMRLWSAK |
| | | APNDFNLKDFNVGGYIQAVLDRNLAENISRVLYPNDNFFEGKELRLKQEYF |
| | | VVAATLQDIIRRFKSSKFGCLDPVRTNFDAFPDKVAIQLNDTHPSLAIPELM |
| | | RILVDQERLEWEKAWEVTVKTCAYTNHTVLPEALERWPVHLIETLLPRHLQ |
| | | IIYEINQRFLNRVAAAFPGDVDRLRRMSLVEEGAVKRINMAHLCIAGSHAV |
| | | NGVARIHSEILKKTIFKDFYELEPHKFQNKTNGITPRRWLVMCNPGLAEIIAE |
| | | RIGEEYIADLDQLRKLLSYVDDESFIRDVAKVKQENKLKFSAYLEKEYKVHI |
| | | NPNSLFDIQVKRIHEYKRQLLNCLHVITLYNRIKKEPNKFFVPRTVMIGGKA |
| | | APGYHMAKMIIKLITAIGDVVNHDPVVGDRLRVIFLENYRVSLAEKVIPAA |
| | | DLSEQISTAGTEASGTGNMKFMLNGALTIGTMDGANVEMAEEAGEENFFIF |
| | | GMRVEDVERLDQKGYNAQEYYDRIPELRHIIDQLSSGFFSPKQPDLFKDIVN |
| | | MLMHHDRFKVFADYEEYVKCQERVSALYKNPREWTRMVIRNIATSGKFSS |
| | | DRTIAQYAREIWGVEPTRQRMPAPDEKI |
| 127 | Bison | MPHQYPALTPEQKKELCDIAHRIVAPGKGILAADESTGSIAKRLQSIGTENTE |
| | A0A6P3H8G9 | ENRRFYRQLLLTADDRVNPCIGGVILFHETLYQKADDGRPFPQVIKAKGGV |
| | (Fructose- | VGIKVDKGVVPLAGTNGETTTQGLDGLSERCAQYKKDGADFAKWRCVLKI |
| | bisphosphate | GEHTPSSLAIMENANVLARYASICQQNGIVPIVEPEILPDGDHDLKRCQYVT |
| | aldolase) | EKVLAAVYKALSDHHIYLEGTLLKPNMVTPGHACTQKYSHEEIAMATVTA |
| | | LRRTVPPAVPGITFLSGGQSEEEASINLNAINKCPLLKPWALTFSYGRALQAS |
| | | ALKAWGGKKENLKAAQEEYVKRALANSLACQGKYTPSGKAGAAASESLFI |
| | | SNHAY |
| 128 | Bison | MAPKKDVKNPAAAAAPAPAPAPAPAPAPAPPKEEKIDLSAIKIEFSKQQQDE |
| | A0A6P3J7N1 | FKEAFLLFDRTGECKITLSQVGDVLRALGTNPTNAEVKKVLGNPSNEEMNA |
| | (Myosin light | KKIEFEQFLPMLQAISNNKDQGTYEDFVEGLRVFDKEGNGTVMGAELRHV |
| | chain 1/3) | LATLGEKMKEEEVEALMAGQEDSNGCINYEAFVKHIMSN |
| 129 | Bison | MVKVGVNGFGHIGRLVTRAAFNSGKVDVIAINDPFIDLHYIVYMFQYDTTH |
| | A0A6P3J4R7 | GKFHGTVKAENGKLIINGKAVTIFQERDPASIKWGDAGAEYVVESTGVFTT |
| | (Glyceraldehyde- | MEKAGAHLKGGAKSVIISAPSADAPMFVLGMNHEKYNNNLKIVSNASCTT |
| | 3-phosphate | NCLAPXAKVIHDHFGIVEGLMTTFHAITVTQKTVDGPSGKLWHDGGGAAQ |
| | dehydrogenase) | NIIPASTGAAKAVGKVIPELNRKLTGMAFCVPTPNVSVMDLTYHLEKPAKY |
| | | DEIKKVVKQTSEGPLEGILGYTQDQVVSCDFNSDSYXSTFDAGAGIALNDH |
| | | FVKLISWYDNEFGYSNRVVDLMVHMASKE |
| 130 | Bison | MDAIKKKMQMLKLDKENALDRAEQAEADKXPVPHGHSQLEDELVSLQKK |
| | A0A6P3GY04 | LKATEDELDKYSEALKDAQEKLELAEKKATDAEADVASLNRRIQLVEEEL |
| | (Tropomyosin | DRAQERLATALQKLEEAEKAADESERGMKVIESRAQKDEEKMEIQEIQLKE |
| | alpha-1 chain | AKHIAEDADRKYEEVARKLVIIESDLERAEERAELSEGKCAELEEELKTVTN |
| | isoform X2) | NLKSLEAQAEKYSQKEDKYEEEIKVLSDKLKEAETRAEFAERSVTKLEKSID |
| | | DLEDELYAQKLKYKAISEELDHALNDMTSM |
| 131 | Bison | MPEAKPAAKKAPAGKDAAAKPAPKEATPQEAPAAPPTGEIDGAPAGRAEP |
| | A0A6P3IB01 | GGSSAASRVVPPRPLLRSVFPTAPGLTALPYSQPCRAPQRFQGGGLTVPLPP |
| | (Myosin-binding | HSPLLPGKDTVIVAKLNGKELPAKPAVKWFKGKWLELGSKSGARFSFKESH |
| | protein C, fast | DAASNVYTIELHITKVVLGDRGDYRIEVKAKDFCDSCAFNIDVEAPRQDSA |
| | type) | GQSLESFKRSGEAKSDTAGELDFSGLLKKRQVVEEEKKKKKDDDDLGIPPEI |
| | | WELLKGAKKSEYERIAFQYGITDLRGMLKRLKKAKVEVKKSAAFTKKLDP |
| | | AYQVDRGNKIKLVVEISDPDLPLKWFKNGQEIKPSSKYVFENVGKKRILTIN |
| | | KCTLADDAAYEVVVKDEKCFTELFVKEPPVLIVTPLEDQQVFVGDRVEMA |
| | | VEVSEEGAQVMWLKDGVELTREDSFKARYRLKKDGKRHILIYSEVTMEDK |
| | | GHYQVMTNGGQCEAELIVEEKQLEVLQDIADLTVKASEQAVFKCEVSDEK |
| | | VTGKWYKNGVEVRPSKRITISHTGRFHKLVIDDVRPEDEGDYTFVPDGYAL |
| | | SLSAKLKFLEIKVEYVPKQEPPKIHLDCSGQTSENAIVVVAGNKLRMDVSIT |
| | | GEPRPVATWMKEDEVFTGTEGRVHIEQRGDISSFVIESAERGDEGRYTIKVT |
| | | NPVGEDVASILLKVVDVPDPPEAVRVTSVGEDWAVLVWEPPKYDGGRPVT |
| | | GYLLERKKKGSQRWMKLNFEVFTETTYESTNMIEGILYEMRVFAVNVIGVS |
| | | QPSMNTKPFMPIAPTSEPRHLTVEDVTDTTTTLKWRPPDKIGAGGIDGYLIE |
| | | YCVEGSDEWIPANTEPVERCGFTVKNLPTGAKITFRVVGVNIAGRSQPATL |
| | | AQPVTIREIVEQPKIRLPRHLRQTYVRKVGEHLNLVIPFQGKPRPQVVWTKG |
| | | GAPIDTSHVHVRTSDFDTVFFVRQAARSDSGEYELTVQIENMKDTATVCIR |
| | | VVEKAGPPQNVMVKEVWGTNALVEWQPPKDDGNSEITGYFVQKADKKT |
| | | MEWFTVYERNRHTSCTVSDLIMGNEYYFRVYSENVCGLSDLPGVSKNTARI |
| | | VKTGMTLKLPEYKEHDFRTPPKFLTPLPDRVVVAGYAAALNCAVRGHPKP |
| | | KVVWMKNKMEIREDPKFLMTNQQGVLTLNIRRPSPFDSGTYSCRAVNELG |
| | | EALAECKLEVRVPQ |
| 132 | Bison | MSLVAVPFYQKRHKHFDQSYRNIQTRYLLDEYSAKKKRASAQSSLQRSVT |
| | A0A6P3GV86 | RKSSSQRESSRAALGESTLGETTLGETSLGDTTLGRTTCRLCAKRMSSALEE |
| | (Myomesin-2) | EAQERKRRYQSMVAAYGEILVRLRSHTVWERMSVKLCFTVQGFPTPVVQ |
| | | WYKDGSLICQAGEPGKYRIDSKYGVHTLEINRADFDDTATYSAVATNVHG |
| | | QVSTNAAVVVRRFRGDEEPFHSVGLPIGLPLSSVIPYTHFDVQFIEKFGVTFR |
| | | REGETLTLKCTLLVTPDLKRVQPRAEWYRDDVLLKESKWTKMFFGEGQAS |
| | | LSFSHLNKDDEGLYTLRIVSRGGITDHSAFLFVRDADPLVTGAPGAPMDVH |
| | | CHDANRDYVIVTWKPPNTTKESPVIGYFIDRCEVGTDNWIQCNDAPVKICK |
| | | YPVTGLFEGRSYIFRMRAVNSAGISRPSRVSEAVAALDPVDLRRLQAVHLE |
| | | GEKDIVIYQEELEGEVQIPGPPTNVHASETSRTYVVLSWDPPEPRGKEPLMY |
| | | FIEKSMVGSGSWQRVNAQTAVRSPRYAVFDLAEGKPYVFRVLSANKHGLS |
| | | DPSEITAPIQAQDTIVVPSAPGRVLASRNTRTSVVVQWDRPKHEEDLLGYY |
| | | VDCSVAGSNVGEPCNHKPIGYNRFVVHGLTTGEQYIFRVKAVNAVGTSENS |
| | | QESDVIKVQAALTVPSHPYGITLLNCDGHSMILGWKVPKFSGGSPILGYYV |
| | | DKREAHHKNWHEVNSSPLKERILTVEGLTEGSLYEFKIAAANMAGIGQPSD |
| | | PSELFKCEAWTMPEPGPAYDLTFCEVRDTSLVLLWKPPVYSGSSPVSGYFV |
| | | DYKEEDSGEWLTVHETATPHRYLKVCDLHQGKTYVFRVRAVNASGVGRP |
| | | SDTSEPVLVEARPGTKEVSAGVDEEGNVYLSFDCPEVTDASHFTWCKSYED |
| | | IADDDRFKVETAGDHSKLYFKNLDKEDLGTYSVSVSDTDGVSSSFVLDEEE |
| | | LERLKALSNEIKNPTIPLKSELAYEIFDKGQVRFWLQAEHLSPDSSYRFIIND |
| | | REVADSETHRIKCDKSTGIIEMVMDRFTIDNEGTYTVQIQDGKAKSQSSLVL |
| | | IGDAFKAVLKEAEFQRKEFLRKQGPHFAEYLHWDVTEECEVRLVCKVANT |
| | | KKETVFKWLKDDVLYETEKMPDLEKGVCELLIPKLSKKDHGEYKATLKDD |
| | | RGQDVSTLEIAGKVYEDMILAMSRVCGASASPLKILCTPEGIRLQCFMKYFT |
| | | EEMKVNWYHKDAKITSSEHMRIGGSEQMAWLQICEPTEKDKGKYTFEILD |
| | | DKKNHQRSLDLSGQAFDEAFAEFQQLKAAAFAEKNRGKVIGGLPDVVTIM |
| | | EGKTLNLTCTVFGNPDPEVVWFKNDKDIELSDHFSVKVEQAKYVSMTIKG |
| | | VTSEDSGKYSIHVKNKYGGEKIDVTVSVYKHGEEIPDVVLPQQAKPKLLPA |
| | | AAPSPAH |
| 133 | Bison | MRTLRAMAMQKIFAREILDSRGNPTVEVDLHTAKGRFRAAVPSGASTGIYE |
| | A0A6P3HSX8 | ALELRDGDKSRYLGKGVLKAVEHINKTLGPALLEKKLSVVDQEKVDKFMI |
| | (Phosphopyruvate | ELDGTENKSKFGANAILGVSLAVCKAGAAEKGVPLYRHIADLAGNPELILP |
| | hydratase) | VPAFNVINGGSHAGNKLAMQEFMILPVGASSFREAMRIGAEVYHHLKGVIK |
| | | AKYGKDATNVGDEGGFAPNILENNEALELLKTAIQAAGYPDKVVIGMDVA |
| | | ASEFYRNGKYDLDFKSPDDPARHISGEKLGELYKNFIKNYPVVSIEDPFDQD |
| | | DWATWTSFLSGVNIQIVGDDLTVTNPKRIAQAVEKKACNCLLLKVNQIGSV |
| | | TESIQACKLAQSNGWGVMVSHRSGETEDTFIADLVVGLCTGQIKTGAPCRS |
| | | ERLAKYNQLMRIEEALGDKAVFAGRKFRNPKAK |
| 134 | Bison | MAPKKAKRRAAAEGGSSSVFSMFDQTQIQEFKEAFTVIDQNRDGIIDKEDL |
| | A0A6P3H0Z6 | RDTFAAMGRLNVKNEELDAMMKEASGPINFTVFLNMFGEKLKGADPEDVI |
| | (Myosin | TGAFKVLDPEGKGTIKKQFLEELLTTQCDRFSHEEIKNMWAAFPPDVGGNV |
| | regulatory light | DYKNICYVITHGDAKDQE |
| | chain 2) | |
| 135 | Bison | MSSDQEMAIFGEAAPYLRKSEKERIEAQNKPFDAKTSVFVADPKESFVKAT |
| | A0A6P3HQ42 | VQSREGGKVTAKTEAGATVTVKEDQVFPMNPPKFDKIEDMAMMTHLHEP |
| | (Myosin-1) | AVLYNLKERYAAWMIYTYSGLFCVTVNPYKWLPVYNAEVVTAYRGKKRQ |
| | | EAPPHIFSISDNAYQFMLTDRENQSILITGESGAGKTVNTKRVIQYFATIAVT |
| | | GEKKKEEPTSGKMQGTLEDQIISANPLLEAFGNAKTVRNDNSSRFGKFIRIH |
| | | FGTTGKLASADIETYLLEKSRVTFQLKAERSYHIFYQIMSNKKPELIEMLLIT |
| | | TNPYDYAYVSQGEITVPSIDDQEELMATDSAIEILGFTSDERVSIYKLTGAV |
| | | MHYGNLKFKQKQREEQAEPDGTEVADKAAYLQGLNSADLLKALCYPRVK |
| | | VGNEFVTKGQTVEQVYNAVGALAKAVYDKMFLWMVARINQQLDTKQPR |
| | | QYFIGVLDIAGFEIFDFNSLEQLCINFTNEKLQQFFNHHMFVLEQEEYKKEGI |
| | | EWEFIDFGMDLAACIELIEKPMGIFSILEEECMFPKATDMSFKNKLYEQHLG |
| | | KSNNFQKPKPAKGKAEAHFSLIHYAGTVDYNITGWLDKNKDPLNETVVGL |
| | | YQKSSVKTLALLFSGPASGEAEGGPKKGGKKKGSSFQTVSALFRENLNKLM |
| | | TNLRSTHPHFVRCIIPNETKTPGAMEHELVLHQLRCNGVLEGIRICRKGFPSR |
| | | ILYADFKQRYKVLNASAIPEGQFIDSKKASEKLLASIDVDHTQYKFGHTKVF |
| | | FKAGLLGLLEEMRDEKLAQLITRTQARCRGFLARVEYQKMVERRESIFCIQ |
| | | YNVRAFMNVKHWPWMKLYFKIKPLLKSAETEKEMANMKEEFEKTKEELA |
| | | KSEAKRKELEEKMVTLTQEKNDLQLQVQSEADALADAEERCDQLIKTKIQL |
| | | EAKIKEVTERAEDEEEINAELTAKKRKLEDECSELKKDIDDLELTLAKVEKE |
| | | KHATENKVKNLTEEMAGLDETIAKLTKEKKALQEAHQQTLDDLQAEEDK |
| | | VNTLTKAKTKLEQQVDDLEGSLEQEKKLRMDLERAKRKLEGDLKLAQEST |
| | | MDIENDKQQLDEKLKKKEFEMSNLQSKIEDEQALAMQLQKKIKELQARIEE |
| | | LEEEIEAERASRAKAEKQRSDLSRELEEISERLEEAGGATSAQIEMNKKREA |
| | | EFQKMRRDLEEATLQHEATAAALRKKHADSVAELGEQIDNLQRVKQKLEK |
| | | EKSEMKMEIDDLASNMETVSKAKGNLEKMCRALEDQLSELKTKEDEQQRL |
| | | INDLTTQRARLQTESGEFSRQLDEKDALVSQLSRGKQAFTQQIEELKRQLEE |
| | | EIKAKSALAHALQSARHDCDLLREQYEEEQEGKAELQRAMSKANSEVAQ |
| | | WRTKYETDAIQRTEELEEAKKKLAQRLQDAEEHVEAVNAKCASLEKTKQR |
| | | LQNEVEDLMIDVERTNAACAALDKKQRNFDKILSEWKQKYEETHAELEAS |
| | | QKESRSLSTELFKIKNAYEESLDQLETLKRENKNLQQEISDLTEQIAEGGKRI |
| | | HELEKVKKQVEQEKSEIQAALEEAEASLEHEEGKILRIQLELNQVKSEIDRKI |
| | | AEKDEEIDQLKRNHIRVVESMQSTLDAEIRSRNDAIRLKKKMEGDLNEMEI |
| | | QLNHANRMAAEALKNYRNTQAILKDTQIHLDDALRGQEDLKEQLAMVER |
| | | RANLLQAEIEELRATLEQTERSRKIAEQELLDASERVQLLHTQNTSLINTKK |
| | | KLETDITQIQGEMEDIIQEARNAEEKAKKAITDAAMMAEELKKEQDTSAHL |
| | | ERMKKNLEQTVKDLQHRLDEAEQLALKGGKKQIQKLEARVRELEGEVESE |
| | | QKRNVEAVKGLRKHERRVKELTYQTEEDRKNILRLQDLVDKLQAKVKSY |
| | | KRQAEEAEEQSNVNLSKFRKLQHELEEAEERADIAESQVNKLRVKSREVHT |
| | | KIISEE |
| 136 | Bison | MADDEEYEEVVEYYTEETVYKEVPGETITGFYETTTTRTSDYEQSETSRPAL |
| | A0A6P3H861 | AQPVPEKPVERKRVIRKKVDPSKFMTPYIAHSQKMQNLFSTNKYKENYEK |
| | (Nebulin isoform | AKGKPYAITTDTPELRRIKKVQDQLSEVKYRVDGDVAKTICHVDEKAKDIE |
| | X11) | HAKKVSQQVSKVLYKQNWEDTKDKYLLPPDAPELVQAIKNTAMFSKKLY |
| | | TEDWEADKTMFYPYNDSPELRRVAQAQKALSDIAYKKGLAEQQTQFTSLP |
| | | DPPDIEFAKKVTNQVSKQKYKEDYENKVKGKWSETPCFEIATARMNSNNIS |
| | | TKKYQEDFEHMKDQIYFMQTETPEYKVNKQAGVAASKVKYKQDYEKNK |
| | | GKADYNVLPASENPLLRQLKAAGDALSDKLYKENYEKTKAKSINYCETPK |
| | | FKLDTVLHNFSSDTKYKDSYLKDILGHYVGSYEDPYHTHCMRVSAQNSDK |
| | | NYKAEYEEDRGKGFFPQTITQEYEAIKKLDQCKDHTYKVHPDKTKFTQVT |
| | | DSPVLMQAQVNSKQLSDLNYKAKHENEKFKCHIPPDTPAFIQHKLNAYNLS |
| | | DNIYKHDWEKTKAKKFDIKVDAIPLLAAKANTKIASDVMYKKDYEKSKGK |
| | | MIGALSINDDPKMLHSLKTAKNQSDRLYKENYEKTKAKSMNYCETPKYQL |
| | | DTLLKNFSEAKYKDSYVQNVLGHYIGSFEDPYQVHCLKISAQNSDKNYKA |
| | | EYEEDKGKCYFPQTITQEYEAIKKLDQCKDHTYKVHPDKTKFTAVTDTPVL |
| | | LQAQLNTKQLSDLNYKAKHEGEKFKCHIPADAPQFIQHRVNAYNLSDNIYK |
| | | HDWEKTKAKKFDIKVDAIPLLAAKANTKIASDVMYKKDYEKSKGKMIGAL |
| | | SINDDPKMLHSLKTAKNQSDHEYRKDYEKSKTIYTAPLDMLPLTHAKKSQ |
| | | AIASDVDYKHLLHNYSYPPDSVNVDLAKKAYALQSDVEYKADYNSWMKG |
| | | CGWMPFGSLEMEKAKRASDILNEKKYRQHPDTLKFTSIEDAPIIVQSKINQA |
| | | QRSDVAYKAKGEEVIHKYSLPADLPQFIQAKVNAYNISENLYKADLKDLSK |
| | | KGYDLRIDAIPIKAAKAARQAASDVQYKKDYEKAKGKMVGFQSLQDDPKL |
| | | VHYMNVAKIQSDREYKKAYEKTKTRHNTPHDMVNIVAAKKAQDVASNV |
| | | NYKHSLHHYTYLPDAMDLELSKNMMHIRSDNVYKEDYNNWMKGIGWIPI |
| | | GSLEVEKVKKAGDALNEKKYRQHPDTLKFTSIVDSPVMVQAKQNTQQVSD |
| | | ILYKAKGEDVKHKYTMSPDLPQFLQAKCNAYNLSDVCYKRDWHDLIAKG |
| | | TNVLGDAIPITAAKASRNIASDYKYKEAYEKAKGKQVGFRSLQDDPKLVH |
| | | YMNVAKLQSDREYKKNYENTKTSYHTPGDMVSITAAKMAQDVATNVNY |
| | | KQPIHHYTYLPDALSLEHIRNVNQIQSDNVYKDEYNHFFKGMGWIPIGSLEV |
| | | EKVKKAGDALNEKKYRQHPDTIKFTSVPDSMGMVLAQHNTKQLSDLNYK |
| | | VEGEKVKHKYTMDPDVPQFIQAKVNAYNMSDSHYKADWKKTLAKGYDL |
| | | RPDAIPIVAAKSSRNIASDFKYKEAYEKTKGKQIGFRSLQDDPKLVHFMNV |
| | | AKMQSDREYKKGYEARKTKYHTPLDMLSVTAAKKSQEVATNTNYKQPFH |
| | | HYTLLPDALNVEHSRNAMQIQSDNLYKSDFTNWMKGIGWLPLESLEVEKA |
| | | KKAGEILSEKKYRQHPEKLKFTYAMDTMEQALNKSNKLIMDKRLYTEKW |
| | | NKDKTTIHVMPDTPDILLSRVNQITMSDKLYKAGWEEEKKKGYDLRPDAIS |
| | | IKAARASRDIASDYKYKQAYEQAKGRQIGFRSLEDDPKLVHFMQVAKMQS |
| | | DREYKKAYEKSKTSFHTPVDMLSVVAAKKSQEVATNANYRNVIHTYNML |
| | | PDAMSLELAKNMMQIQSDNQYKADYADFMKGIGWLPLGSLEAEKNKKA |
| | | MEIISEKKYRQHPDTLKYSTLMDSMNMVLAKNNAKIMNEHLYKQAWEAD |
| | | KTKVHIMPDIPQIILAKANAINISDKLYKLSLEEAKKKGYDLRTDAIPIKAAK |
| | | ASRDIASDYKYKHSYEKERGKMVGFRSLEDDPKLVHSMQVAKMQSDREY |
| | | KKNYEKTKTSYHTPADMLSVTAAKDAQANITNTNYKHLIHKYILLPDAMNI |
| | | QLSKNMNRIQSDNEYKQDYNEWYKGLGWSPAGSLEVEKAKKATEYASDQ |
| | | KYRQHPSNFQFTKLNDSMDMVLAKQNAHTMNKYLYTIDWNKDKTKIHV |
| | | MPDTPDILQAKQNQTMYSQKLYKLGWEEALKKGYDLPVDAISVQAAKAS |
| | | RDIVSDFKYKQGYRKQIGHHIGFRSLQDDPKLVLSMNVAKMQSEREYKKD |
| | | FEKWKTKFSSPVDMLGVVLAKKCQALVSDVDYKNYLHGWTCLPDQNDVI |
| | | HAKKAYDLQSENLYKSDLEWLKGIGWSPLGSLEAEKNKRASEIISEKKYRQ |
| | | PPDRNKFTSIPDAMDIVLAKTNAKNRSDILYREAWDKDKTQIHIMPDTPDIIL |
| | | AKANLINTSDKFYRMGYEELRKKGYDLPVDAIPIKAAKASREIASEYKYKE |
| | | GFRMQLGHHIGARNIKDDPKMMWSMHVAKIQSDREYKKDFEKWKTKFSS |
| | | PVDMLGVVLAKKCQTLVSDIDYKNYLHQWTCLPDQSDVIHARRAYDLQS |
| | | DNLYKSDLQWLRGIGWLPSGSLEDEKNKRASQILSDHVYRQHPDQFKFSSL |
| | | MDSIPMVLAKNNAITMNHRLYTEAWDKDKTTVHIMPDTPEVLLAKQNQIN |
| | | YSEKLYKLGLDEAKRKGYDMRIDAIPIRAAKASRDIASEFKYKEGYRRQLG |
| | | HHIGARAIHDDPKMMWSMHVAKIQSDREYKKDFEKWKTKFSSPVDMLGV |
| | | VLAKKCQTLVSDIDYKNYLHQWTCLPDQSDVIHARRAYDLQSDNVYKSDL |
| | | QWLRGIGWVPIGSLDVEKSKRASEILSDKLYRQPPDKFKFTSVTDSLEQVLA |
| | | KNNAINMNKRLYTEAWDKDKTQVHIMPDTPEITLARTNKVNYSENLYKLA |
| | | HEEAKKKGYDLRSDAIPIVAAKASRDIISDYKYKDGYRKQLGHHIGARDIK |
| | | DDPKMMWSMHVAKIQSDREYKKDFEKWKTKFSSPVDMLGVVLAKKCQT |
| | | LVSDIDYKNYLHQWTCLPDQSDVIHARRAYDLQSDNVYKSDLQWLRGIG |
| | | WVPIESVDVVKCKRAAEILSDNLYRQPPDTLKFTSVPDSLEQVLAKNNAIN |
| | | MNKRLYTEAWDKDKTQIHIMPDTPEIMLARQNKLNYSETLYKLANEEAKK |
| | | KGYDLRSDAIPIVAAKASRDIISDYKYKDGYRKQLGHHIGARDIKDDPKMM |
| | | WSMHVAKIQSDREYKKDFEKWKTKFSSPVDMLGVVLAKKCQTLVSDIDY |
| | | RNYLHQWTCLPDQSDVIHARQAYDLQSDNIYKSDLQWLRGIGWVPIGSVD |
| | | VVKCKRAAEILSDNLYRQRPDTLKFTSVPDSLEQVLAKNNAINMNKRLYTE |
| | | AWDKDKTQIHMMPDTPEITLARQNKINYSENLYRQAMEEAKKEGYDLRSD |
| | | AIPIVAAKASREIASDYKYKEAYRKQLGHHIGARAIHDDPKMMWSVHVAK |
| | | MQSDREYKKDFEKYKTRFSSPVDMLGIVLAKKCQTLVSDVDYKHPLHEWT |
| | | CLPDQNDIIHARKAYDLQSDNLYKSDLEWLKGIGWVPIGSVEVLKAKRAGE |
| | | ILSDNIYRQRPDTLKFTSVTDSPEQVLAKNNAINMNKRLYTEAWDNDKKTI |
| | | HVMPDTPEIMLAKLNRINYSDKLYKLALEESRKEGYDLRLDAIPIQAAKAS |
| | | REIASDYKYKEGYRKQLGHHIGARNIKDDPKMMWSIHAGKIQSDLEYKKD |
| | | FEKWKTKFSSPVDMMGLVQAKKCQILVSDIDYKNLLHEWTCLPDQNDIIQ |
| | | ARKAYDLQSDAIYKADLEWLRGIGWVPIDSVGVEHAKRAGEILSERKYRQP |
| | | AVQLKFTSITDTPEIVLAKNNALNVSKHLYTEAWDADKTSIHVMPDTPEILL |
| | | AKSNSANISHKLYTKGWDESKMKDYDLRADAISIKSAKASRDIASDYKYKE |
| | | AYEKHKGHHIGARSVEDDPRIMCAMNAGRIQSEREYKKEFQKWKTKFSSP |
| | | VDMLGILLAKKCQTLVSDIDYRNYLHHWTCLPDQNDIIQARKAYDLQSDAI |
| | | YKADLEWLRGIGWMPEGSPEVLRVKNAQHIFRDSVYRTPVVKLKYTSIVD |
| | | TPEVVLAKSNAENISIPKYREVWDKDKTSIHIMPDTPEINLARTNALNVSNK |
| | | LYREGWDEMKMSCDVRLDAIPIQAAKASREIASDYKYKLDHEKQKGHYV |
| | | GTRTARDDNKIRWALIAGKIQNEREYRLHWAKWKSKFQSPPDMLSIEHSK |
| | | NSQTLVSDIDYRHYLHQWTCMPDQNDVIQARKAYDLQSDAIYKADLEWL |
| | | RGIGWMPADSVSVNHAKHMADIFNEKKYRTKIETLSFTPVDDRVDYVTAK |
| | | HSGEILNDIKYRKDWNDTKSKYTLTETPQLHTAQEAARILDQYLYKESWEK |
| | | QKATGYILPPDAVPFVHAHHSGDVQSELKYKAEHVKQKGHYVGVPTMRD |
| | | DPKLVWFEHAGQIQNDRLYKENYHKTKAKIHIPPDMVSVLAAKEGQALAS |
| | | DIDYRNYLHQWICHPDQNDVIQARKAYDLQSDNIYKADLEWLRGIGWIPL |
| | | DSVDHVRVTRNQEMMNQIKYKKDALANYPNFTSVVDPPEIVLAKINSVNQ |
| | | SDVKYKETFNKLIKGKYIFSPDTPYITHSKDMEKLYSTILYKRAWDGTKAY |
| | | GYTLDERYIPIVGAKHADFVNSELKYKETYEKLKGHYLAGKEISEFPNVVH |
| | | CLDFQKMRSLLNYRRHYEDTKANVHIPQDMMNHVLAKRCQYILSDLEYR |
| | | HYFHQWTSLPEEPNVVRVRHAQEILSDNVYKDDLNWLKGIGCYVWDTPQI |
| | | LHAKKSYDLQSQILYTAAGKENLKNYNLVTDTPLYVTALQSGINASEVKY |
| | | KENYHQIKDKYTTVLETVDYDRIKNLKDLFSSNLYKEAWDKVKATSYILPP |
| | | NTVSLTHAKNQKYMASHIKYREEYEKFKALYTLPRSVEDDPNTARCLRVG |
| | | KFNIDRLYRSVYEKNKMKIHIVPDMVEMVTAKDSQKKVSEIDYRLHLHEWI |
| | | CHPDLQVNSHVRKVTDQISDIVYKDDLTWLKGIGCYVWDTPEILHAKHAY |
| | | DLRNDIKYKAHVLKTRNNYKLVTDTPVYVQAVKSGKQLSDAVYHYDYVH |
| | | SIRGRVAPTTKTVDLDRALHAYKLQSENLYRKAGLHALPTGYRLPVDTPHF |
| | | KHTKDTRYMSSYFKYKEVYEHMKAYGYTLGPNDVPFVNVRRVNNITSERL |
| | | YRQLYHKLKDKIHTTPDTPEIRQVKKTQEAVSELIYKSDFFKMQGHMISLPY |
| | | TPQVLHCRYVGDITSDIKYKEDLQVLRGMGCFLYDTPDMVRSRHLRKLWS |
| | | HYLYTDKARKMRDKYKVVLDTPEYRKVQELKTHLSELVYRAAGRKQKSIF |
| | | TSVPDTPDLTRAKRGQKLQSQYLYVELATKERPHHHAGNQTTALKHARHV |
| | | KDMVSENKYKIQYEKMKDKYTPVPDTPILIRAKRAYWNASDLRYKETFQK |
| | | TKGKYHTVKDALDIVYHRTVTDHVSKIKYKENYMSQLGIWRSIPDRPEHFH |
| | | HRAVTDAVSDVKYKQDLTWLKGIGCYAYDTPDFTLAEKNKTLYSKYKYK |
| | | EVFERTKSNFKYVADCPINRHFKFATQLMNEKKYRADYEQRKDKYHLVVD |
| | | EPRHLLAKIAGDQISQIKYRKNYEETKDKFTSIVDTPEHLRTTKVNKQISDIL |
| | | YKLEYNKAKPRGYTTIHDTPMLLHVRKVKDEVSDLKYKEVYQRTKSNCTI |
| | | EPDAVHIKAAKDAYKVNTNLDYKKKYEATKAYWKWTPDRPDFIQAAKST |
| | | LQQSDFEYKLDREYLKGCKLSVTDDKDMVLALKNSIIESDLKYKEKHVKE |
| | | RGSCHAVPDTPQILLAKTVSSLVSENKYKSYVKKHLAQGSYTTLPETRDTIH |
| | | VKEVTKNVSDTNYKKKFVKEKGKSNYSIMLEPPDVKHAMDVAKKQSNVA |
| | | YKKDAKENLHYTTVADRPDIKKATQAAKQASEVEYRAKHRKEGSHGLSM |
| | | LGRPDIEMAKKAAKLSSQVQYRENFNKEKGKTPKYNPKDSQLYKVMKDA |
| | | NTLASEVKYKADLKKLHKPVTDMKESLIMNHVLNTSHLASSYQYKKNYEK |
| | | SKGHYHTIPDNLEQLHLKEATELQSIVKYKEKYEKERGKPMLDFETPTYITA |
| | | KESQQMQSGKEYRKDYEESIKGRNLTGLEVTPALLHVKYATKIASEKEYRK |
| | | DLEESIRGKGLSEMEDTPDMLRAKNATQILNEKEYKRDLELEVKGRGLNA |
| | | MANETPDFLRARNATDIASQIKYKQSAEMEKANFTSVVDTPEIIHAQQVKN |
| | | LSSQKKYKEDAEKCMSYYETVLDTPEMQRVRENQKNFSLLQYQYDLKNS |
| | | KGKITVVQDTPEILRVKENQKNFSSVLYKEDVSPGTAIGKTPEMMRVKQTQ |
| | | DHISSVKYKEVIGQGTPIPDLPEVKRVKQTQKHISSVMYKENLGTGIPTPVTP |
| | | EIERVKRNQENFSSVLYKENLGKGTPTPITPEMERVKRNQENFSSVLYKEN |
| | | MGKGTPLPVTPEMERIKHNQENISSVLYKENVGKGTPTPVTPEMQRVKRNQ |
| | | ENISSVLYKENLGKATPTPFTPEMERVKRNQENFSSVLYKENMRKATPTPV |
| | | TPEMERAKRNQENISSVLYSDSFRKQIQGKAAYVLDTPEMRRVRETQRHIS |
| | | TVKYHEDFEKHKGCFTPVVTDPITERVKKNTQDFSDICYRGIQRKVVEMEQ |
| | | KRNDQDQETITGLRVWRTNPGSVFDYDPAEDNIQSRSLHMINAQAQRRSRE |
| | | QSRSASGLSISGGEEKSEHSEAAHLSTYSDGGVFFSAASTGYKHARTTELPQ |
| | | QRSSSVATQQTTVSSIPSHPSTAGKIFRAMYDYMAADADEVSFKDGDAIVN |
| | | VQAIDEGWMYGTVQRTGRTGMLPANYVEAI |
| 137 | Bison | MSSDQEMAIFGEAAPYLRKSEKERIEAQNKPFDAKTSVFVVDPKESFVKAT |
| | A0A6P3HPK5 | VQSREGGKVTAKTEAGATVTVKEDQIFPMNPPKYDKIEDMAMMTHLHEPA |
| | (Myosin-4) | VLYNLKERYAAWMIYTYSGLFCVTVNPYKWLPVYNAEVVTAYRGKKRQE |
| | | APPHIFSISDNAYQFMLTDRENQSILITGESGAGKTVNTKRVIQYFATIAVTG |
| | | EKKKEEPTSGKMQGTLEDQIISANPLLEAFGNAKTVRNDNSSRFGKFIRIHF |
| | | GTTGKLASADIETYLLEKSRVTFQLKAERSYHIFYQIMSNKKPELIEMLLITT |
| | | NPYDYAFVSQGEITVPSIDDQEELIATDSAIDILGFTSDERVSIYKLTGAVMH |
| | | YGNLKFKQKQREEQAEPDGTEVADKAAYLQSLNSADLLKALCYPRVKVG |
| | | NEFVTKGQTVQQVYNAVGALAKAVYEKMFLWMVTRINQQLDTKQPRQY |
| | | FIGVLDIAGFEIFDFNSLEQLCINFTNEKLQQFFNHHMFVLEQEEYKKEGIEW |
| | | EFIDFGMDLAACIELIEKPMGIFSILEEECMFPKATDTSFKNKLYEQHLGKSN |
| | | NFQKPKPAKGKAEAHFSLVHYAGTVDYNIAGWLDKNKDPLNETVVGLYQ |
| | | KSAMKTLAFLFAGGPSAEEGGGGKKGGKKKGSSFQTVSALFRENLNKLMT |
| | | NLRSTHPHFVRCIIPNETKTPGAMEHELVLHQLRCNGVLEGIRICRKGFPSRI |
| | | LYADFKQRYKVLNASAIPEGQFIDSKKASEKLLGSIDIDHTQYKFGHTKVFF |
| | | KAGLLGILEEMRDEKLAQLITRTQAICRGFLMRVEFRKMMERRESIFCIQYN |
| | | VRAFMNVKHWPWMKLYFKIKPLLKSAETEKEMANMKEEFEKTKEELAKS |
| | | EAKRKELEEKMVTLMQEKNDLQLQVQSEADGLADAEERCDQLIKTKIQLE |
| | | AKIKELTERAEDEEEINAELTAKKRKLEDECSELKKDIDDLELTLAKVEKEK |
| | | HATENKVKNLTEEMAGLDENIAKLTKEKKALQEAHQQTLDDLQAEEDKV |
| | | NTLTKSKTKLEQQVDDLEGSLEQEKKLRMDLERAKRKLEGDLKLAQESTM |
| | | DIENDKQQLDEKLKKKEFEMSNLQSKIEDEQALAMQLQKKIKELQARIEEL |
| | | EEEIEAERASRAKAEKQRSDLSRELEEISERLEEAGGATSAQIEMNKKREAE |
| | | FQKMRRDLEEATLQHEATAAALRKKHADSVAELGEQIDNLQRVKQKLEKE |
| | | KSEMKMEIDDLASNMETVSKAKGNLEKMCRTLEDQLSEVKSKEEEHQRLI |
| | | NELSAQKARLHAESGEFSRQLEEKDALVSQLSRGKQAFTQQIEELKRQLEE |
| | | ETKAKSALAHALQSARHDCDLLREQYEEEQEGKAELQRALSKANSEVAQ |
| | | WRTKYETDAIQRTEELEEAKKKLAQRLQDAEEHVEAVNAKCASLEKTKQR |
| | | LQNEVEDLMLDVERSNAACAALDKKQRNFDKVLSEWKQKYEETQAELEA |
| | | SQKESRSLSTELFKVKNACEESLDQLETLKRENKNLQQEISDLTEQIAEGGK |
| | | HIHELEKVKKQIEQEKGELQAALEEAEASLEHEEGKILRIQLELNQVKSEIDR |
| | | KIAEKDEEIDQLKRNHLRVMESTQSTLDAEIRSRNDALRIKKKMEGDLNEM |
| | | EIQLNHANRQATEAIKNLRNTQGVLKDTQLHLDDAIRGQDDLKEQLAMVE |
| | | RRANLMQAEIEELRASLEQTERSRRVAEQELLDASERVQLLHTQNTSLINTK |
| | | KKLETDITQIQGEMEDILQEARNAEEKAKKAITDAAMMAEELKKEQDTSA |
| | | HLERMKKNMEQTVKDLQHRLDEAEQLALKGGKKQIQKLEARVRELENEV |
| | | ENEQKRNAEAVKGLRKHERRVKELTYQTEEYRKNVLRLQDLVDKLQSKV |
| | | KAYKRQAEEAEEQSNVNLAKFRKLQHELEEAEERADIAESQVNKLRVKSR |
| | | EVHTKVISEE |
| 138 | Bison | MKWVTFISLLLLFSSAYSRGVFRRDTHKSEIAHRFKDLGEEHFKGLVLIAFS |
| | A0A6P3HBK1 | QYLQQCPFDEHVKLVNELTEFAKTCVADESHAGCEKSLHTLFGDELCKVA |
| | (Serum albumin) | SLRETYGNMADCCEKQEPERNECFLSHKDDSPDLPKLKPDPNTLCDEFKAD |
| | | EKKFWGKYLYEIARRHPYFYAPELLYYANKYNGVFQECCQAEDKGACLLP |
| | | KIETMREKVLTSSARQRLRCASIQKFGERALKAWSVARLSQKFPKAEFVEV |
| | | TKLVTDLTKVHKECCHGDLLECADDRADLAKYICDNQDTISSKLKECCDKP |
| | | LLEKSHCIAEVEKDAVPENLPPLTADFAEDKDVCKNYQEAKDAFLGSFLYE |
| | | YSRRHPEYAVSVLLRLAKEYEATLEECCAKDDPHACYSTVFDKLKHLVDE |
| | | PQNLIKQNCDQFEKLGEYGFQNALIVRYTRKVPQVSTPTLVEVSRSLGKVG |
| | | TRCCTKPESERMPCTEDYLSLILNRLCVLHEKTPVSEKVTKCCTESLVNRRP |
| | | CFSALTPDETYVPKAFDEKLFTFHADICTLPDTEKQIKKQTALVELLKHKPK |
| | | ATEEQLKTVMENFVAFVDKCCAADDKEACFAVEGPKLVVSTQTALA |

An animal protein can comprise a deer protein (Table 7). A deer protein can comprise a deer muscle protein. A deer protein can comprise a deer blood protein. A deer protein can comprise a deer albumin protein. A deer protein can comprise a sequence that has at least or about 70%, at least or about 75%, at least or about 80%, at least or about 90%, at least or about 91%, at least or about 92%, at least or about 93%, at least or about 94%, at least or about 95%, at least or about 96%, at least or about 97% at least or about 98% at least or about 99% or about 100% sequence identity to any one of SEQ ID NO: 139-147.

A food product may comprise a fragment or a truncation of a deer protein. The food product may include a fragment or truncation of any one of SEQ ID Nos: 139-147. A sequence of the fragment or truncation may be identical or substantially identical to a portion of any one of SEQ ID Nos: 139-147. A sequence of the fragment or truncation may be at least about 70%, 80%, 90%, 95%, 98%, or more identical to a portion of a sequence of any one of SEQ ID Nos: 139-147. A length of the fragment or truncation may be at least about 10%, 15%, 20%, 30%, 40%, 50%, 60%, 70%, 80%, 90%, or more of a length of any one of SEQ ID Nos: 139-147. A length of the fragment or truncation may be less than or equal to about 90%, 80%, 70%, 60%, 50%, 40%, 30%, 20%, 15%, 10%, or less than a length of any one of SEQ ID Nos: 139-147.

TABLE7
Deer Proteins

| SEQ | | |
|---|---|---|
| ID NO | Description | Amino Acid Sequence |
| 139 | Deer | MLDAEDIVNTPKPDEKAIMTYVSCFYHAFAGAEQAETAANRICKVLAVNQ |
| | A0A212DH10 | ENEKLMEEYEKLASELLEWIRRTVPWLENRVGEPSMSAMQRKLEDFRDYR |
| | (ACTN3) | RLHKPPRVQEKCQLEINFNTLQTKLRLSHRPAFMPSEGKLVSDIAKAWRGL |
| | | EQVEKGYEDWLLSEIRRLQRLQHLAEKFQQKASLHEAWTRGKEEMLSQRD |
| | | YETASLQEVRALLRRHEAFESDLAAHQDRVEHIAALAQELNELDYHEAAS |
| | | VNSRCQAICDQWDNLGTLTQKRRDALERMEKLLETIDQLQLEFARRAAPF |
| | | NNWLDGAVEDLQDVWLVHSVEETQSLVTAHDQFKATLPEADRERGAILGI |
| | | QGEIQKICQTYGLRPSSTNPYIALTPQDINTKWDTVRKLVPSRDQMLQEELT |
| | | RQQVNERLRRQFAAQANAIGPWIQGKVEEVGRLAAGMAGSLEEQMAGLR |
| | | QQEQNIINYKSNIDRLEGDHQLLQESLVFDNKHTVYSMEHIRVGWEQLLTSI |
| | | ARTINEVENQVLTRDAKGLSQEQLNEFRASFNHFDRKRNGMMEPDDFRAC |
| | | LISMGYDLGEVEFARIMTMVDPNAAGVVTFQAFIDFMTRETAETDTAEQV |
| | | VASFKILAGDKNYITAEELRRELPAEQAEYCIRRMAPYKGAGAPAGALDYV |
| | | AFSSALYGESDL |
| 140 | Deer | MSRPLTDQEKRKQISVRGLAGVENVTELKKNFNRHLHFTLVKDRNVATPR |
| | A0A212DGZ3 | DYYFALAYTVRDHLVGRWIRTQQHYYEKDPKRIYYLSLEFYMGRTLQNTM |
| | (Alpha-1,4 glucan | VNLALENACDEATYQLGLDMEELEEIEEDAGLGNGGLGRLAACFLDSMAT |
| | phosphorylase) | LGLAAYGYGIRYEFGIFNQKISGGWQMEEADDWLRYGNPWEKARPEFTLP |
| | | VHFYGRVEHTSQGAKWVDTQVVLAMPYDTPVPGYRNNVVNTMRLWSAK |
| | | APNDFNLKDFNVGGYIQAVLDRNLAENISRVLYPNDNFFEGKELRLKQEYF |
| | | VVAATLQDIIRRFKSSKFGCLDPVAIQLNDTHPSLAIPELMRILVDQERLEWE |
| | | KAWEVTVKTCAYTNHTVLPEALERWPVHLIETLLPRHLQIIYEINQRFLNRV |
| | | AAAFPGDVDRLRRMSLVEEGAVKRINMAHLCIAGSHAVNGVARIHSEILKK |
| | | TMTSKSWSLINSRIRPMGSPLGAGWLCRIGEEYIADLDQLRKLLSYVDDESF |
| | | IRDVAKVKQENKLKFSAYLEKEYKVHINPNSLFDIQVKRIHEYKRQLLNCL |
| | | HAAPGYHMAKMIIKLITAIGDVVNHDPVVGDRLRVIFLENYRVSLAEKVIP |
| | | AADLSEQISTAGTEASGTGNMKFMLNGALTIGTMDGANVEMAEEAGEENF |
| | | FIFGMRVEDVERLDQRGYNAQEYYDRIPELRHVIDQLSSGFFSPKQPDLFKD |
| | | IVNMLMHHDRFKVFADYEEYVKCQERVSALYKNPREWTRMVIRNIATSGK |
| | | FSSDRTIAQYAREIWGVEPNRQRMPAPDEKI |
| 141 | Deer | MAQRKPEGSGFHMTCLSMALAYSFRPDANIQPHPQLGNTQKQTELGKELT |
| | A0A212CZ49 | GTMPHQYPALTPEQKKELCDIAHRIVAPGKGILAADESTGSIAKRLQSIGTE |
| | (Fructose- | NTEENRRFYRQLLLTADDRVNPCIGGVILFHETLYQKADDGRPFPQVIKAK |
| | bisphosphate | GGVVDKGVVPLAGTNGETTTQGLDGLSERCAQYKKDGADFAKWRCVLKI |
| | aldolase) | GEHTPSSLAIMENANVLARYASICQQNGIVPIVEPEILPDGDHDLKRCQYVT |
| | | EKVLAAVYKALSDHHIYLEGTLLKPNMVTPGHACTQKYSHEEIAMATVTA |
| | | LRRTVPPAVPGITFLSGGQSEEEASINLNAINKCPLLKPWALTFSYGRALQAS |
| | | ALKAWGGKKENLKAAQEEYVKRALANSLACQGKYTPSGKAGAAASESLFI |
| | | SNHAY |
| 142 | Fragment of Deer | PAPAPPKEEKIDLSAIKIEFSKQQQDEFKEAFLLFDRTGECKITLNQVGDVLR |
| | A0A212D3R8 | ALGTNPTNAEVKKVLGNPSNEEMNAKKIEFEQFLPMLQAISNNKDQGTYE |
| | (MYL1) | DFVEGLRVFDKEGNGTVMGAELRHVLATLGEKMKEEEVEALMAGQEDSN |
| | | GCINYEAFVKHIMSN |
| 143 | Deer | MFVMGVNHEKYNNTLKIVSNASCTTNCLAPLAKVIHDHFGIVEGLMTTVH |
| | A0A212CCI7 | AITATQKTVDGPSGKLWRDGRGAAQNIIPASTGAAKAVGKVIPELNGKLTG |
| | (GAPDH) | MAFRVPTPNVSVVDLTCRLEKPAKYDEIKKVVKQASEGPLKGILGYTEDQV |
| | | VSCDFNSDTHSSTFDAGAGIALNDHFVKLISWYDNEFGYSNRVVDLMVHM |
| | | ASKE |
| 144 | Deer | MRTLRAMAMQKIFAREILDSRGNPTVEVDLHTAKGRFRAAVPSGASTGIYE |
| | A0A212D927 | ALELRDGDKSRYLGKGVLKAVEHINKTLGPALLEKASLGRLYLLPPAKFGA |
| | (Phosphopyruvate | NAILGVSLAVCKAGAAEKGVPLYRHIADLAGNPELILPVPAFNVINGGSHA |
| | hydratase) | GNKLAMQEFMILPVGASSFKEAMRIGAEVYHHLKGVIKAKYGKDATNVG |
| | | DEGGFAPNILENNEALELLKTAIQAAGYPDKVVIGMDVAASEFYRNGKYDL |
| | | DFKSPDDPARHISGEKLGELYKNFIKNYPVVSIEDPFDQDDWATWTSFLSGV |
| | | NIQIVGDDLTVTNPKRIAQAVEKKACNCLLLKVNQIGSVTESIQACKLAQSN |
| | | GWGVMVSHRSGETEDTFIADLVVGLCTGQIKTGAPCRSERLAKYNQLMRIE |
| | | EALGDKAVFAGRKFRNPKAK |
| 145 | Deer | MFDQTQIQEFKEAPKKAKRRAAAEGGSSSVFSMFDQTQIQEFKEAFTVIDQ |
| | A0A212CY74 | NRDGIIDKEDLRDTFAAMGRLNVKNEELDAMMKEASGPINFTVFLNMFGE |
| | (MYLPF) | KLKGADPEDVITGAFKVLDPEGKGTIKKQFLEELLTTQCDRFSQEEMRNM |
| | | WAAFPPDVGGNVDYKNICYVITHGDAKDQE |
| 146 | Deer | MCEEEDSTALVCDNGSGLCKAGFAGDDAPRAVFPSIVGRPRHQGVMVGM |
| | A0A212CNJ3 | GQKDSYVGDEAQSKRGILTLKYPIEHGIITNWDDMEKIMFETFNVPAMYVA |
| | (Actin) | IQAVLSLYASGRTTGIVLDSGDGVTHNVPIYEGYALPHAIMRLDLAGRDLT |
| | | DYLMKILTERGYSFVTTAEREIVRDIKEKLCYVALDFENEMATAASSSSLEK |
| | | SYELPDGQVITIGNERFRCPETLFQPSFIGMESAGIHETTYNSIMKCDIDIRKD |
| | | LYANNVLSGGTTMYPGIADRMQKEITALAPSTMKIKIIAPPERKYSVWIGGS |
| | | ILASLSTFQQMWISKQEYDEAGPSIVHRKCF |
| 147 | Deer | MKWVTFISLLLLFSSAYSRGVFRRDTHKSEIAHRFKDLGEDNFQGLVLIAFS |
| | A0A212D5P0 | QYLQQCPFDEHVKLVKELTDFAKECVADESHAGCEKSLHTLFGDELCKVA |
| | (ALB) | SLRETYGDMADCCEKQEPERNECFLKHKDDNPDLPKLKPDPDTLCAEFKA |
| | | DEKKFWGKYLYEVARRHPYFYAPELLYYANKYNGVFQECCQADDKAACL |
| | | LPKIDTMREKVLTSSARQRLKCASIQKFGERALKAWTVALLSQKFPKADFA |
| | | EVTKIVTDLTKVHKECCHGDLLECADDRADLAKYICDNQDTLSSKLKECC |
| | | DKPVLEKSHCIAEIEKDAVPENLPPLTADFAEDKEVCKNYQEAKDVFLGTF |
| | | LYEYARRHPEYAVSVLLRVAKEYEATLEDCCAKDDPHACYATVFDKLQHL |
| | | VDEPQNLIKQNCDLFEKVGEYGFQNALIVRYTKKAPQVSTPTLVEVSRNLG |
| | | RVGTKCCTKPESERMSCTEDYLSLVLNRLCVLHEKTPVSEKVTKCCTESLV |
| | | NRRPCFSALTLDETYVPKAFDEKLFTFHADICTVPDAEKQIKKQTALVELLK |
| | | HKPKATDEQLKTVMENFVAFVDKCCAAVDKEACFAVEPTLRVRGKKEK |

An animal protein can comprise a chicken protein (Table 8). A chicken protein can comprise a chicken muscle protein. A chicken protein can comprise a chicken blood protein. A chicken protein can comprise a chicken albumin protein. A chicken protein can comprise a sequence that has at least or about 70%, at least or about 75%, at least or about 80%, at least or about 90%, at least or about 91%, at least or about 92%, at least or about 93%, at least or about 94%, at least or about 95%, at least or about 96%, at least or about 97% at least or about 98% at least or about 99% or about 100% sequence identity to any one of SEQ ID NO: 148-161.

A food product may comprise a fragment or a truncation of a chicken protein. The food product may include a fragment or truncation of any one of SEQ ID Nos: 148-161. A sequence of the fragment or truncation may be identical or substantially identical to a portion of any one of SEQ ID Nos: 148-161. A sequence of the fragment or truncation may be at least about 70%, 80%, 90%, 95%, 98%, or more identical to a portion of a sequence of any one of SEQ ID Nos: 148-161. A length of the fragment or truncation may be at least about 10%, 15%, 20%, 30%, 40%, 50%, 60%, 70%, 80%, 90%, or more of a length of any one of SEQ ID Nos: 148-161. A length of the fragment or truncation may be less than or equal to about 90%, 80%, 70%, 60%, 50%, 40%, 30%, 20%, 15%, 10%, or less than a length of any one of SEQ ID Nos: 148-161.

TABLE8
Chicken Proteins

| SEQ | | |
|---|---|---|
| ID NO | Description | Amino Acid Sequence |
| 148 | Chicken KCRM | MPFSSTHNKHKLKFSAEEEFPDLSKHNNHMAKVLTPELYKRLRDKETPSGF |
| | (Creatine kinase | TLDDVIQTGVDNPGHPFIMTVGCVAGDEESYEVFKDLFDPVIQDRHGGYKP |
| | M-type) | TDKHRTDLNHENLKGGDDLDPKYVLSSRVRTGRSIKGYSLPPHCSRGERRA |
| | | VEKLSVEALNSLEGEFKGRYYPLKAMTEQEQQQLIDDHFLFDKPVSPLLLA |
| | | SGMARDWPDARGIWHNDNKTFLVWVNEEDHLRVISMEKGGNMKEVFRRF |
| | | CVGLKKIEEIFKKAGHPFMWTEHLGYILTCPSNLGTGLRGGVHVKLPKLSQ |
| | | HPKFEEILHRLRLQKRGTGGVDTAAVGAVFDISNADRLGFSEVEQVQMVV |
| | | DGVKLMVEMEKKLEQNQPIDDMIPAQK |
| 149 | Chicken (ACTN2 | MNSMNQIETNMQYTYNYEEDEYMTQEEEWDRDLLLDPAWEKQQRKTFTA |
| | Alpha-actinin-2) | WCNSHLRKAGTQIENIEEDFRNGLKLMLLLEVISGERLPKPDRGKMRFHKI |
| | | ANVNKALDYIASKGVKLVSIGAEEIVDGNVKMTLGMIWTIILRFAIQDISVE |
| | | ETSAKEGLLLWCQRKTAPYRNVNIQNFHLSWKDGLGLCALIHRHRPDLIDY |
| | | SKLNKDDPIGNINLAMEIAEKHLDIPKMLDAEDIVNTPKPDERAIMTYVSCF |
| | | YHAFAGAEQAETAANRICKVLAVNQENERLMEEYERLASELLEWIRRTIPW |
| | | LENRTPEKTMQAMQKKLEDFRDYRRKHKPPKVQEKCQLEINFNTLQTKLRI |
| | | SNRPAFMPSEGKMVSDIAGAWQRLEQAEKGYEEWLLNEIRRLERLEHLAE |
| | | KFRQKASTHEQWAYGKEQILLQKDYESASLTEVRAMLRKHEAFESDLAAH |
| | | QDRVEQIAAIAQELNELDYHDAASVNDRCQKICDQWDSLGTLTQKRREAL |
| | | ERTEKLLETIDQLHLEFAKRAAPFNNWMEGAMEDLQDMFIVHSIEEIQSLIS |
| | | AHDQFKATLPEADGERQAILSIQNEVEKVIQSYSMRISASNPYSTVTVEEIRT |
| | | KWEKVKQLVPQRDQSLQEELARQHANERLRRQFAAQANVIGPWIQTKME |
| | | EIARSSIEMTGPLEDQMNQLKQYEQNIINYKHNIDKLEGDHQLIQEALVFDN |
| | | KHTNYTMEHIRVGWELLLTTIARTINEVETQILTRDAKGITQEQMNDFRASF |
| | | NHFDRRKNGLMDHDDFRACLISMGYDLGEAEFARIMSLVDPNGQGTVTFQ |
| | | SFIDFMTRETADTDTAEQVIASFRILASDKPYILADELRRELPPEQAQYCIKR |
| | | MPQYTGPGSVPGALDYTSFSSALYGESDL |
| 150 | Chicken E1BSN7 | MAAPLSDGERRKQISVRGIAGLGDVAEVRKSFNRHLHFTLVKDRNVATPR |
| | (Alpha-1,4 glucan | DYFFALAHTVRDHLVGRWIRTQQHYYERDPKRIYYLSLEFYMGRTLQNTM |
| | phosphorylase) | VNLGLQNACDEAIYQLGLDLEELEEIEEDAGLGNGGLGRLAACFLDSMATL |
| | | GLAAYGYGIRYEFGIFNQKIIDGWQVEEADDWLRYGNPWEKARPEYMLPV |
| | | HFYGRVEHSPEGVKWVDTQVVLAMPYDTPVPGYKNNTVNTMRLWSAKA |
| | | PNDFNLQEFNVGDYIEAVLDRNLAENISRVLYPNDNFFEGKELRLKQEYFV |
| | | VAATLQDIIRRFKSSKFGCQDPVRTSFETFPDKVAIQLNDTHPALSIPELMRIL |
| | | VDVEKVDWDKAWEITKQTCAYTNHTVLPEALERWPVSMFEKLLPRHLEII |
| | | YALNQMHLDRVAALYPGDVDRLRRMSVIEEGDCKRINMAHLCVIGSHAV |
| | | NGVARIHSDIVKNTVFKDFYELEPEKFQNKTNGITPRRWLLLCNPGLADVIA |
| | | EKIGEDFVTDLSQLKKLLDFINNETFIRDVAKVKQENKLKFSAYLEERYKVK |
| | | INPSSMFDIQVKRIHEYKRQLLNCLHAITLYNRIRSDPSKSFVPRTIMIGGKA |
| | | APGYHMAKMIIKLITSIGEVVNNDPYVGDKLKVIFLENYRVSLAEKVIPAAD |
| | | LSQQISTAGTEASGTGNMKFMLNGALTIGTMDGANVEMAEEAGEENLFIFG |
| | | MRVEEVEALDRRGYNAREYYDRIPELRQAIDQISSGFFSPRDPGCFRDVVN |
| | | MLMHHDRFKVFADYEAYIKCQGQVDQLFMDPREWTKKVIRNIACSGKFSS |
| | | DRTITEYAREIWGVEPSATKIPPPNLPRD |
| 151 | Chicken R4GM10 | MTHQYPALTAEQKKELSDIALRIVAPGKGILAADESVGSMAKRLNQIGVEN |
| | (Fructose- | TEENRRLYRQILFSADSRVKKCIGGVIFFHETMYQKADDGTPFVQMIKDKGI |
| | bisphosphate | VVGIKVDKGVVPLAGTDGETTTQGLDGLSERCAQYKKDGADFAKWRCVL |
| | aldolase) | KISDNTPSALAIMENANVLARYASICQQNGIVPIVEPEILPDGDHDLKRCQY |
| | | VTEKVLAAVYKALSDHHVYLEGTLLKPNMVTPGHSCPTKYSPEEIAMATV |
| | | TALRRTVPPAVPGVTFLSGGQSEEEASINLNAINTCPLVRPWALTFSYGRAL |
| | | QASALSAWRGQRDNANAATEEFVKRAEVNGLAALGKYEGSGDDSGAAGQ |
| | | SLYVANHAY |
| 152 | Chicken MLE1 | MAPKKDVKKPAAAAAPAPAPAPAPAPAPAKPKEPAIDLKSIKIEFSKEQQD |
| | (Myosin light | DFKEAFLLFDRTGDAKITLSQVGDIVRALGQNPTNAEINKILGNPSKEEMNA |
| | chain 1) | KKITFEEFLPMLQAAANNKDQGTFEDFVEGLRVFDKEGNGTVMGAELRHV |
| | | LATLGEKMTEEEVEELMKGQEDSNGCINYEAFVKHIMSV |
| 153 | Chicken G3P | MVKVGVNGFGRIGRLVTRAAVLSGKVQVVAINDPFIDLNYMVYMFKYDST |
| | (Glyceraldehyde- | HGHFKGTVKAENGKLVINGHAITIFQERDPSNIKWADAGAEYVVESTGVFT |
| | 3-phosphate | TMEKAGAHLKGGAKRVIISAPSADAPMFVMGVNHEKYDKSLKIVSNASCT |
| | dehydrogenase) | TNCLAPLAKVIHDNFGIVEGLMTTVHAITATQKTVDGPSGKLWRDGRGAA |
| | | QNIIPASTGAAKAVGKVIPELNGKLTGMAFRVPTPNVSVVDLTCRLEKPAK |
| | | YDDIKRVVKAAADGPLKGILGYTEDQVVSCDFNGDSHSSTFDAGAGIALND |
| | | HFVKLVSWYDNEFGYSNRVVDLMVHMASKE |
| 154 | Chicken TPM1 | MDAIKKKMQMLKLDKENALDRAEQAEADKKAAEERSKQLEDELVALQK |
| | (Tropomyosin | KLKGTEDELDKYSESLKDAQEKLELADKKATDAESEVASLNRRIQLVEEEL |
| | alpha-1 chain) | DRAQERLATALQKLEEAEKAADESERGMKVIENRAQKDEEKMEIQEIQLKE |
| | | AKHIAEEADRKYEEVARKLVIIEGDLERAEERAELSESKCAELEEELKTVTN |
| | | NLKSLEAQAEKYSQKEDKYEEEIKVLTDKLKEAETRAEFAERSVTKLEKSID |
| | | DLEDELYAQKLKYKAISEELDHALNDMTSI; |
| 155 | Chicken MYPC2 | MPEPSKAAPKKEAKKKEEKKEEKKEAPPPQEHKDEAPDDVHPPETPDPEGL |
| | (Myosin-binding | FLSKPQNVMVESGRDVTVSARVAGAALPCAPAVKWFKGKWAELGDKSAR |
| | protein C) | CRLRHSVDDDKVHTFELTITKVAMGDRGDYRCEVTAKEQKDSCSFSIDVEA |
| | | PRSSEGNVLQAFKRTGEGKDDTAGELDFSGLLKKREVQVEEKKKKKDEDD |
| | | QFPPEIWELLKGVTKKSEYERIAFQYGITDLRGMLKRLKKVHVEPKKSEAFI |
| | | RKLDPAYQVDKGNKIKLVVELSDPDLPLKWYKNGQLLKPSTKYVFENVGL |
| | | KRILTIHKCSLADDAAYECRVNDEKCFTEVFVKEPPVTVVRGLEDQQVVVG |
| | | DRVVLEAEVSEEGAQVMWLKDGVDVTRDDAFKYRFKKDGKKHFLIINEA |
| | | ELSDSAHYKIMTNGGESEAELSVEEKQLEVLQDMADLTVKASEQAVFKCE |
| | | VSDEKVTGRWFRNGVEVKPSKRIHISHNGRFHKLVIDDVRPEDEGDYTFIPD |
| | | GYALSLSAKLNFLEIKVEYVPKQEPPKIHLDCSGKAAENTIVVVAGNKVRL |
| | | DVPISGEPAPTVTWKRGDQLFTATEGRVHIDSQADLSSFVIESAERSDEGRY |
| | | CITVTNPVGEDSATLHVRVVDVPDPPQSVRVTSVGEDWAVLSWEAPPFDG |
| | | GMPITGYLMERKKKGSMRWMKLNFEVFPDTTYESTKMIEGVFYEMRVFA |
| | | VNAIGVSQPSLNTQPFMPIAPTSEPTHVVLEDVTDTTATIKWRPPERIGAGG |
| | | VDGYLVEWCREGSNEWVAANTELVERCGLTARGLPTGERLLFRVISVNMA |
| | | GKSPPATMAQPVTIREIVERPKIRLPRHLRQTYIRRVGEQVNLVIPFQGKPRP |
| | | QVTWSREGGALPAEVQTRTSDVDSVFFIRSAARPLSGNYEMRVRIDNMEDC |
| | | ATLRLRVVERPGPPQAVRVMEVWGSNALLQWEPPKDDGNAEISGYTVQK |
| | | ADTRTMEWFTVLEHSRPTRCTVSELVMGNEYRFRVYSENVCGTSQEPATS |
| | | HNTARIAKEGLTLKMVPYKERDLRAAPQFLTPLVDRSVVAGYTVTLNCAV |
| | | RGHPKPKVTWLKNSVEIGADPKFLSRHGLGVLSLLIRRPGPFDGGTYGCRA |
| | | VNEMGEATTECRLDVRVPQ |
| 156 | Chicken MPSF | MSSVAVPFYQRRHKHFDQSYRNIQTRYVLEEYAARKAASRQAAHYESTGL |
| | (M-protein) | GKTTCRLCARRARSLAHEAMQESRKRTHEQKSHASDEKRIKFASELSSLER |
| | | EIHMARHHAREQLDRLAIQRMVEENMALERHVVEEKISRAPEILVRLRSHT |
| | | VWEKMSVRLCFTVQGFPSPVVQWYKNEELITPASDPAKYSVENKYGVHVL |
| | | HINRADFDDSATYSAVATNIHGQASTNCAVVVRRFRESEEPHPAGIMPFHLP |
| | | LSYDVCFTHFDVQFLEKFGVTFATEGETLTLKCSVLVTPELKRLRPRAEWY |
| | | RDDVLIKDSKWTKLYFGEGQAALSFTHLNKDDEGLYTLRMVTKGGVNECS |
| | | AFLFVRDADALIAGAPGAPMDVKCHDANRDYVIVTWKPPNTTSQNPVIGY |
| | | FVDKCEVGLENWVQCNDAPVKICKYPVTGLYEGRSYIFRVRAVNSAGISRP |
| | | SRVSEPVAALDPVDLERTQTVHVDEGRKIVISKDDLEGDIQIPGPPTNVHAS |
| | | EISKTYVVLSWDPPVPRGREPLTYFIEKSMVGSGSWQRVNAQVAVKSPRYA |
| | | VFDLAEGKPYVFRVLSANKHGISDPSEITEPIQPQDIVVVPSAPGRVVATRNT |
| | | KTSVVVQWDKPKHEENLYGYYIDYSVVGSNQWEPANHKPINYNRFVVHG |
| | | LETGEQYIFRVKAVNAVGFSENSQESEAIKVQAALTCPSYPHGITLLNCDGH |
| | | SMTLGWKAPKYSGGSPILGYYIDKREANHKNWHEVNSSVISRTIYTVEDLT |
| | | EDAFYEFKIAAANVVGIGHPSDPSEHFKCKAWTMPEPGPAYDLTVCEVRNT |
| | | SLVLLWKAPVYEGKSPITGYLVDYKEVDTEDWITANEKPTSHRYFKVTDLH |
| | | QGHTYVFKVRAVNDAGVGKSSEISEPVFVEASPGTKEIFSGVDEEGNIYLGF |
| | | ECKEATDASHFLWGKSYEEIEDSDKFKIETKGDHSKLYFKHPDKSDLGTYCI |
| | | SVSDTDGVSSSFVLDEEELERLMTLSNEIKNPTIPLKSELAYEVLDKGEVRF |
| | | WIQAESLSPNSTYRFVINDKEVENGDRHKISCDHSNGIIEMVMDKFTIDNEG |
| | | TYTVQIQDGKAKNQSSLVLIGDAFKAILAESELQRKEFLRKQGPHFSEFLYW |
| | | EVTEECEVLLACKIANTKKETVFKWYRNGSGIDVDEAPDLQKGECHLTVPK |
| | | LSRKDEGVYKATLSDDRGHDVSTLELSGKVYNDIILALSRVSGKTASPLKIL |
| | | CTEEGIRLQCFLKYYNEEMKVTWSHRESKISSGEKMKIGGGEDVAWLQITE |
| | | PTEKDKGNYTFEIFSDKESFKRTLDLSGQAFDDALTEFQRLKAAAFAEKNR |
| | | GKVIGGLPDVVTIMDGKTLNLTCTVFGNPDPEVVWFKNDKALELNEHYLV |
| | | SLEQGKYASLTIKGVTSEDSGKYSIYVKNKYGGETVDVTVSVYRHGEKIPE |
| | | VNQGQLAKPRLIPPSSST |
| 157 | Chicken ENOB | MSIQKIHAREILDSRGEPTVEVDLHTAKGHFRAAVPSGASTGIHEALEPRDG |
| | (Beta-enolase) | DKKRFLGKGVLKAVEHINKTIGPALIEKKISVVEQEKIDKVMIEMDGTENKS |
| | | KFGANAILGVSLAVCKAGAAEKGVPLYRHIADLAGNTELILPVPAFNVING |
| | | GSHAGNKLAMQEFMVLPVGAASFHDAMRVGAEVYHSLKGVIKAKYGKD |
| | | ATNVGGEGGFAPNILDNHEALELLKAAIAQAGYTDKVVIGMDVAASEFCR |
| | | DGRYHLDFKSPPHTKRYITGEQLGEIYRGFIKDYPVVSIEDPFDQDDWEAW |
| | | KRFVFHVDIQVVGDDLTVTNPKRIAHGAEQHACNCLLLKVNQIGSVTESIQ |
| | | ACKLAQSHGWGVMVSHRSGETEDTFIADLVVGLCTGQIKTGAPCRSERLA |
| | | KYNQLMRIEEALGDKAKFAGRKFRNPKAK |
| 158 | Chicken MYL11 | MAPKKAKRRAAEGSSNVFSMFDQTQIQEFKEAFTVIDQNRDGIIDKDDLRE |
| | (Myosin | TFAAMGRLNVKNEELDAMIKEASGPINFTVFLTMFGEKLKGADPEDVIMG |
| | regulatory light | AFKVLDPDGKGSIKKSFLEELLTTQCDRFTPEEIKNMWAAFPPDVAGNVDY |
| | chain 11) | KNICYVITHGEDKEGE |
| 159 | Chicken MYH1B | MATDADMAIFGEAAPYLRKSEKERIEAQNKPFDAKSSVFVVHAKESYVKST |
| | (Myosin-1B) | IQSKESGKVTVKTEGGETLTVKEDQIFSMNPPKYDKIEDMAMMTHLHEPAV |
| | | LYNLKERYAAWMIYTYSGLFCVTVNPYKWLPVYNPEVVLAYRGKKRQEA |
| | | PPHIFSISDNAYQFMLTDRENQSILITGESGAGKTVNTKRVIQYFATIAASGD |
| | | KKKEEQPAGKMQGTLEDQIISANPLLEAFGNAKTVRNDNSSRFGKFIRIHFG |
| | | ATGKLASADIETYLLEKSRVTFQLKAERSYHIFYQIMSNKKPELIEMLLITTN |
| | | PYDYQYVSQGEITVPSINDQEELMATDSAIDILGFTPDEKTAIYKLTGAVMH |
| | | YGNLKFKQKQREEQAEPGGTEVADKAAYLMGLNSADLLKALCYPRVKVG |
| | | NEYVTKGQTVQQVYNSVGALAKSVFEKMFLWMVVRINQQLDTKQPRQYF |
| | | IGVLDIAGFEIFDFNSLEQLCINFTNEKLQQFFNHHMFVLEQEEYKKEGIEWE |
| | | FIDFGMDLAACIELIEKPMGIFSILEEECMFPKATDTSFKNKLYDQHLGKSNN |
| | | FQKPKPGKGKAEAHFSLVHYAGTVDYNITGWLEKNKDPLNETVVGLYQKS |
| | | SLKTLALLFASVGGAEAESGAGGKKGGKKKGSSFQTVSALFRENLNKLMS |
| | | NLRSTHPHFVRCLIPNETKTPGAMEHELVLHQLRCNGVLEGIRICRKGFPIRI |
| | | LYADFKQRYKVLNASAIPEGQFIDSKKASEKLLGSIDVDHTQYKFGHTKVF |
| | | FKAGLLGLLEEMRDEKLAQLITRTQARCRGFLMRVEFKKMMERRESIFCIQ |
| | | YNVRAFMNVKHWPWMKLFFKIKPLLKSAESEKEMANMKEEFEKTKEELA |
| | | KSEAKRKELEEKMVSLLQEKNDLQLQVQAEADGLADAEERCDQLIKTKIQ |
| | | LEAKIKELTERAEDEEEMNAELTAKKRKLEDECSELKKDIDDLELTLAKVE |
| | | KEKHATENKVKNLTEEMAALDETIAKLTKEKKALQEAHQQTLDDLQAEED |
| | | KVNTLTKAKTKLEQQVDDLEGSLEQEKKLRMDLERAKRKLEGDLKMTQE |
| | | STMDLENDKQQLDEKLKKKDFEISQIQSKIEDEQALGMQLQKKIKELQARIE |
| | | ELEEEIEAERTSRAKAEKHRADLSRELEEISERLEEAGGATAAQIDMNKKRE |
| | | AEFQKMRRDLEEATLQHEATAAALRKKHADSTADVGEQIDNLQRVKQKL |
| | | EKEKSELKMEIDDLASNMESVSKAKANLEKMCRSLEDQLSEIKTKEEEQQR |
| | | TINDISAQKARLQTESGEYSRQVEEKDALISQLSRGKQAFTQQIEELKRHLEE |
| | | EIKAKKCPAHALQSARHDCDLLREQYEEEQEAKGELQRALSKANSEVAQW |
| | | RTKYETDAIQRTEELEEAKKKLAQRLQDAEEHVEAVNSKCASLEKTKQRL |
| | | QNEVEDLMIDVERSNAACAALDKKQKNFDKILSEWKQKYEETQAELEASQ |
| | | KESRSLSTELFKMKNAYEESLDHLETLKRENKNLQQEISDLTEQIAEGGKAI |
| | | HELEKVKKQIEQEKSELQTALEEAEASLEHEEGKILRVQLELNQVKSDIDRK |
| | | IAEKDEEIDQLKRNHLRVVDSMQSTLDAEIRSRNEALRLKKKMEGDLNEIEI |
| | | QLSHANRQAAEAQKNLRNTQGVLKDTQIHLDDALRSQEDLKEQVAMVER |
| | | RANLLQAEIEELRAALEQTERSRKVAEQELLDASERVQLLHTQNTSLINTKK |
| | | KLESDISQIQSEMEDTIQEARNAEEKAKKAITDAAMMAEELKKEQDTSAHL |
| | | ERMKKNLDQTVKDLQHRLDEAEQLALKGGKKQIQKLEARVRELEGEVDA |
| | | EQKRSAEAVKGVRKYERRVKELTYQSEEDRKNVLRLQDLVDKLQMKVKS |
| | | YKRQAEEAEELSNVNLSKFRKIQHELEEAEERADIAESQVNKLRAKSREIGK |
| | | KAESEE |
| 160 | Chicken R4GIG1 | MATDADMAIFGEAAPYLRKSEKERIEAQNKPFDAKSSVFVVHAKESYVKST |
| | (Uncharacterized | IQSKESGKVTVKTEGGETLTVKEDQIFSMNPPKYDKIEDMAMMTHLHEPAV |
| | protein) | LYNLKERYAAWMIYTYSGLFCVTVNPYKWLPVYNPEVVLAYRGKKRQEA |
| | | PPHIFSISDNAYQFMLTDRENQSILITGESGAGKTVNTKRVIQYFATIAASGD |
| | | KKKEEQPAGKMQGTLEDQIISANPLLEAFGNAKTVRNDNSSRFGKFIRIHFG |
| | | ATGKLASADIETYLLEKSRVTFQLKAERSYHIFYQIMSNKKPELIEMLLITTN |
| | | PYDYQYVSQGEITVPSINDQEELMATDSAIDILGFTPDEKTAIYKLTGAVMH |
| | | YGNLKFKQKQREEQAEPDGTEVADKAAYLMGLNSADLLKALCYPRVKVG |
| | | NEYVTKGQTVQQVYNSVGALAKSVFEKMFLWMVVRINQQLDTKQPRQYF |
| | | IGVLDIAGFEIFDFNSLEQLCINFTNEKLQQFFNHHMFVLEQEEYKKEGIEWE |
| | | FIDFGMDLAACIELIEKPMGIFSILEEECMFPKATDTSFKNKLYDQHLGKSNN |
| | | FQKPKPGKGKAEAHFSLVHYAGTVDYNITGWLEKNKDPLNETVVGLYQKS |
| | | SLKTLALLFASVGGAEAESGAGGKKGGKKKGSSFQTVSALFRENLNKLMS |
| | | NLRSTHPHFVRCLIPNETKTPGAMEHELVLHQLRCNGVLEGIRICRKGFPSRI |
| | | LYADFKQRYKVLNASAIPEGQFIDSKKASEKLLGSIDVDHTQYKFGHTKVF |
| | | FKAGLLGLLEEMRDEKLAQLITRTQARCRGFLMRVEFKKMMERRESIFCIQ |
| | | YNVRAFMNVKHWPWMKLFFKIKPLLKSAESEKEMANMKEEFEKTKEELA |
| | | KSEAKRKELEEKMVSLLQEKNDLQLQVQAEADGLADAEERCDQLIKTKIQ |
| | | LEAKIKELTERAEDEEEMNAELTAKKRKLEDECSELKKDIDDLELTLAKVE |
| | | KEKHATENKVKNLTEEMAALDETIAKLTKEKKALQEAHQQTLDDLQAEED |
| | | KVNTLTKAKTKLEQQVDDLEGSLEQEKKLRMDLERAKRKLEGDLKMTQE |
| | | STMDLENDKQQLDEKLKKKDFEISQIQSKIEDEQALGMQLQKKIKELQARIE |
| | | ELEEEIEAERTSRAKAEKHRADLSRELEEISERLEEAGGATAAQIDMNKKRE |
| | | AEFQKMRRDLEEATLQHEATAAALRKKHADSTAELGEQIDNLQRVKQKLE |
| | | KEKSELKMEIDDLASNMESVSKAKANLEKMCRSLEDQLSEIKTKEEEQQRT |
| | | INDISAQKARLQTESGEYSRQVEEKDALISQLSRGKQAFTQQIEELKRHLEEE |
| | | IKAKNALAHALQSARHDCDLLREQYEEEQEAKGELQRALSKANSEVAQWR |
| | | TKYETDAIQRTEELEEAKKKLAQRLQDAEEHVEAVNSKCASLEKTKQRLQ |
| | | NEVEDLMIDVERSNAACAALDKKQKNFDKILSEWKQKYEETQAELEASQK |
| | | ESRSLSTELFKMKNAYEESLDHLETLKRENKNLQQEISDLTEQIAEGGKAIH |
| | | ELEKVKKQIEQEKSELQAALEEAEASLEHEEGKILRVQLELNQVKSDIDRKI |
| | | AEKDEEIDQLKRNHLRVVDSMQSTLDAEIRSRNEALRLKKKMEGDLNEIEI |
| | | QLSHANRQAAEAQKNLRNTQGVLKDTQIHLDDALRSQEDLKEQVAMVER |
| | | RANLLQAEIEELRAALEQTERSRKVAEQELLDASERVQLLHTQNTSLINTKK |
| | | KLESDISQIQSEMEDTIQEARNAEEKAKKAITDAAMMAEELKKEQDTSAHL |
| | | ERMKKNLDQTVKDLQHRLDEAEQLALKGGKKQIQKLEARVRELEGEVDA |
| | | EQKRSAEAVKGVRKYERRVKELTYQSEEDRKNVLRLQDLVDKLQMKVKS |
| | | YKRQAEEAEELSNVNLSKFRKIQHELEEAEERADIAESQVNKLRAKSREIGK |
| | | KAESEE |
| 161 | Chicken ALBU | MKWVTLISFIFLFSSATSRNLQRFARDAEHKSEIAHRYNDLKEETFKAVAMI |
| | (Albumin) | TFAQYLQRCSYEGLSKLVKDVVDLAQKCVANEDAPECSKPLPSIILDEICQV |
| | | EKLRDSYGAMADCCSKADPERNECFLSFKVSQPDFVQPYQRPASDVICQEY |
| | | QDNRVSFLGHFIYSVARRHPFLYAPAILSFAVDFEHALQSCCKESDVGACLD |
| | | TKEIVMREKAKGVSVKQQYFCGILKQFGDRVFQARQLIYLSQKYPKAPFSE |
| | | VSKFVHDSIGVHKECCEGDMVECMDDMARMMSNLCSQQDVFSGKIKDCC |
| | | EKPIVERSQCIMEAEFDEKPADLPSLVEKYIEDKEVCKSFEAGHDAFMAEFV |
| | | YEYSRRHPEFSIQLIMRIAKGYESLLEKCCKTDNPAECYANAQEQLNQHIKE |
| | | TQDVVKTNCDLLHDHGEADFLKSILIRYTKKMPQVPTDLLLETGKKMTTIG |
| | | TKCCQLGEDRRMACSEGYLSIVIHDTCRKQETTPINDNVSQCCSQLYANRR |
| | | PCFTAMGVDTKYVPPPFNPDMFSFDEKLCSAPAEEREVGQMKLLINLIKRK |
| | | PQMTEEQIKTIADGFTAMVDKCCKQSDINTCFGEEGANLIVQSRATLGIGA |

An animal protein can comprise a turkey protein (Table 9). A turkey protein can comprise a turkey muscle protein. A turkey protein can comprise a turkey blood protein. A turkey protein can comprise a turkey albumin protein. A turkey protein can comprise a sequence that has at least or about 70%, at least or about 75%, at least or about 80%, at least or about 90%, at least or about 91%, at least or about 92%, at least or about 93%, at least or about 94%, at least or about 95%, at least or about 96%, at least or about 97% at least or about 98% at least or about 99% or about 100% sequence identity to any one of SEQ ID NO: 162-174.

A food product may comprise a fragment or a truncation of a turkey protein. The food product may include a fragment or truncation of any one of SEQ ID Nos: 162-174. A sequence of the fragment or truncation may be identical or substantially identical to a portion of any one of SEQ ID Nos: 162-174. A sequence of the fragment or truncation may be at least about 70%, 80%, 90%, 95%, 98%, or more identical to a portion of a sequence of any one of SEQ ID Nos: 162-174. A length of the fragment or truncation may be at least about 10%, 15%, 20%, 30%, 40%, 50%, 60%, 70%, 80%, 90%, or more of a length of any one of SEQ ID Nos: 162-174. A length of the fragment or truncation may be less than or equal to about 90%, 80%, 70%, 60%, 50%, 40%, 30%, 20%, 15%, 10%, or less than a length of any one of SEQ ID Nos: 162-174.

TABLE9
Turkey Proteins

| SEQ | | |
|---|---|---|
| ID NO | Description | Amino Acid Sequence |
| 162 | Turkey G1NL16 | MPFSNSHNLLKMKHSADDEFPDLSAHNNHMAKVLTLDLYKKLRDRQTSSG |
| | (Creatine kinase) | FTLDDVIQTGVDNPGHPFIMTVGCVAGDEESYEVFKELFDPVIEDRHGGYK |
| | | PTDEHKTDLNADNLQGGDDLDPNYVLSSRVRTGRSIRGFCFPHCSRGERRA |
| | | IEKLSVEALGSLGGDLKGKYYALRNMTDAEQQQLIDDHFLFDKPVSPLLLA |
| | | SGMARDWPDARGIWHNDNKTFLVWINEEDHLRVISMQKGGNMKEVFTRF |
| | | CTGLTQIETLFKSKNYEFMWNPHLGYILTCPSNLGTGLRAGVHIKLPNLGK |
| | | HEKFGEVLKRLRLQKRGTGGVDTAAVGGVFDVSNADRLGFSEVELVQMV |
| | | VDGVKLLIEMEKRLEKGQSIDDLMPAQK |
| 163 | Turkey | MNSMNQIETNMQYTYNYEEDEYMTQEEEWDRDLLLDPAWEKQQRKTFTA |
| | A0A803Y9V3 | WCNSHLRKAGTQIENIEEDFRNGLKLMLLLEVISGERLPKPDRGKMRFHKI |
| | (Actinin alpha 2) | ANVNKALDYIASKGVKLVSIGAEEIVDGNVKMTLGMIWTIILRFAIQDISVE |
| | | ETSAKEGLLLWCQRKTAPYRNVNIQNFHLSWKDGLGLCALIHRHRPDLIDY |
| | | SKLNKDDPIGNINLAMEIAEKHLDIPKMLDAEDIVNTPKPDERAIMTYVSCF |
| | | YHAFAGAEQAETAANRICKVLAVNQENERLMEEYERLASELLEWIRRTIPW |
| | | LENRTPEKTMQAMQKKLEDFRDYRRKHKPPKVQEKCQLEINFNTLQTKLRI |
| | | SNRPAFMPSEGKMVSDIAGAWQRLEQAEKGYEEWLLNEIRRLERLEHLAE |
| | | KFRQKASTHEQWAYGKEQILLQKDYESASLTEVRAMLRKHEAFESDLAAH |
| | | QDRVEQIAAIAQELNELDYHDAASVNDRCQKICDQWDSLGTLTQKRREAL |
| | | ERTEKLLETIDQLHLEFAKRAAPFNNWMEGAMEDLQDMFIVHSIEEIQSLIS |
| | | AHDQFKATLPEADGERQAILSIQNEVEKVIQSYSMRISASNPYSTVTVEEIRT |
| | | KWEKVKQLVPQRDQSLQEELARQHANERLRRQFAAQANVIGPWIQTKME |
| | | EIARSSIEMTGPLEDQMNQLKQYEQNIINYKHNIDKLEGDHQLIQEALVEDN |
| | | KHTNYTMEHIRVGWELLLTTIARTINEVETQILTRDAKGITQEQMNDFRASF |
| | | NHFDRRKNGLMDHDDFRACLISMGYDLGEAEFARIMSLVDPNGQGTVTFQ |
| | | SFIDFMTRETADTDTAEQVIASFRILASDKPYILADELRRELPPEQAQYCIKR |
| | | MPQYTGPGSVPGALDYTSFSSALYGESDL |
| 164 | Turkey | MAAPLSDGERRKQISVRGIAGLGDVAEVRKSFNRHLHFTLVKDRNVATPR |
| | A0A803Y8V3 | DYFFALAHTVRDHLVGRWIRTQQHYYERDPKRIYYLSLEFYMGRTLQNTM |
| | (Alpha-1,4 glucan | VNLGLQNACDEAIYQLGLDLEELEEIEEDAGLGNGGLGRLAACFLDSMATL |
| | phosphorylase) | GLAAYGYGIRYEFGIFNQKIVDGWQVEEADDWLRYGNPWEKARPEYMLP |
| | | VHFYGRVEHSPEGVKWVDTQVVLAMPYDTPVPGYKNNTVNTMRLWSAK |
| | | APNDFNLQEFNVGDYIEAVLDRNLAENISRVLYPNDNFFEGKELRLKQEYF |
| | | VVAATLQDIIRRFKSSKFGCQDPVRTSFETFPDKVAIQLNDTHPALSIPELMR |
| | | ILVDVEKVDWDKAWEITKQTCAYTNHTVLPEALERWPVSMFEKLLPRHLEI |
| | | IYALNQMHLDRVAALYPGDIDRLRRMSVIEEGDCKRINMAHLCVIGSHAVN |
| | | GVARIHSDIVKNTVFKDFYELDPEKFQNKTNGITPRRWLLLCNPGLADVIAE |
| | | KIGEDFVTDLSQLKKLLDFIDDETFIRDVAKVKQENKLKFSAYLEEHYKVKI |
| | | NPSSMFDIQVKRIHEYKRQLLNCLHAITLYNRKSDPSKSFVPRTIMIGGKAA |
| | | PGYHMAKMIIKLITSIGEVVNNDPYVGDKLKVIFLENYRVSLAEKVIPAADL |
| | | SQQISTAGTEASGTGNMKFMLNGALTIGTMDGANVEMAEEAGEENLFIFG |
| | | MRVEEVEALDRQGYNAREYYDRIPELRQAIDQISSGFFSPRDPGCFRDVVN |
| | | MLMHHDRFKVFADYEAYIKCQGQVDQLFMDPREWTKKVIRNIACSGKFSS |
| | | DRTITEYAREIWGVEPSGDVFPAWEA |
| 165 | Turkey GIN2A4 | MTHQYPALTAEQKKELSDIALRIVAPGKGILAADESVGSMAKRLNQIGVEN |
| | (Fructose- | TEENRRLYRQILFSADSRVKKCIGGVIFFHETMYQKADDGTPFVQMIKDKGI |
| | bisphosphate | VVGIKVDKGVVPLAGTDGETTTQGLDGLSERCAQYKKDGADFAKWRCVL |
| | aldolase) | KISDNTPSALAIMENANVLARYASICQQNGIVPIVEPEILPDGDHDLKRCQY |
| | | VTEKVLAAVYKALSDHHVYLEGTLLKPNMVTPGHSCPTKYSPEEIAMATV |
| | | TALRRTVPPAVPGVTFLSGGQSEEEASINLNAINTCPLVRPWALTFSYGRAL |
| | | QASALSAWRGQRDNANAATEEFVKRAEVNGLAALGKYEGSGDDSGAAGQ |
| | | SLYVANHAY |
| 166 | Turkey Q6W5H1 | MAPKKDVKKPAAAAAPAPAPAPAPAPAPAKPKEPAIDLKSIKIEFSKEQQD |
| | (Myosin essential | DFKEAFLLFDKTGDAKITLSQVGDIVRALGQNPTNAEMNKILGNPSKEEMN |
| | light chain | AKKITFEEFLPMLQAAANNKDQGTYEDFVEGLRVFDKEGNGTVMGAELRH |
| | isoform 1) | VLATLGEKMTEEEVEELMKGQEDSNGCINYEAFVKHIMSV |
| 167 | Turkey G1NMR6 | MVKVGVNGFGRIGRLVTRAAVLSGKVQVVAINDPFIDLNYMVYMFKYDST |
| | Glyceraldehyde- | HGHFKGTVKAENGKLVINGHAITIFQERDPSNIKWADAGAEYVVESTGVFT |
| | (3-phosphate | TMEKAGAHLKGGAKRVIISAPSADAPMFVMGVNHEKYDKSLKIVSNASCT |
| | dehydrogenase) | TNCLAPLAKVIHDNFGIVEGLMTTVHAITATQKTVDGPSGKLWRDGRGAA |
| | | QNIIPASTGAAKAVGKVIPELNGKLTGMAFRVPTPNVSVVDLTCRLEKPAK |
| | | YDDIKRVVKAAADGPLKGILGYTEDQVVSCDFNGDSHSSTFDAGAGIALND |
| | | HFVKLVSWYDNEFGYSNRVVDLMVHMASKE |
| 168 | Turkey | MEAIKKKMQMLKLDKENALDRAEQAEAEQKQAEERSKQLEDELAAMQK |
| | A0A803XNQ2 | KLKGTEDELDKYSEALKDAQEKLELAEKKAADAEAEVASLNRRIQLVEEE |
| | (Tropomyosin 3) | LDRAQERLATALQKLEEAEKAADESERGMKVIENRALKDEEKMELQEIQL |
| | | KEAKHIAEEADRKYEEVARKLVIIEGDLERTEERAELAESKCSELEEELKNV |
| | | TNNLKSLEAQAEKYSQKEDKYEEEIKILTDKLKEAETRAEFAERSVAKLEK |
| | | TIDDLEDELYAQKLKYKAISEELDHALNDMTSI |
| 169 | Turkey | MCLQLFVFSELPPPFSFLKNKELTFVYCLHKALMVECWCHSICSSPTLADQI |
| | A0A803YPL8 | REVVGLESLVSSGKCILERIKHWLIIDIFLFLCISVWSIGESPPEETEKHDDSQ |
| | (Myosin-binding | RSTLFIEKPQSGSVSVGGNITFIAKVEAKDLLRKPNVKWFKGKWMDLASKA |
| | protein C) | GKHLQLKESFERHTKIHTFEMHIIQAKENYAGNYRCEVSYKDKFDSCSFDL |
| | | EVTESSQAAPSIDIRSAFKRSGDGQDDAGELDFSGLLKRREIKQQEEEPEVD |
| | | VWELLKNANPSEYEKIAFQYGITDLRGMLKRLKRMRREVKKSAAFSKGLD |
| | | PAYQVDKGGKVRFMVELADPTVELKWYKNGQEIRPSAKYIFEHKGNQRIL |
| | | FINNCTMTDDARYYVTAGDEKCSTELFVRDPPILVTKGLEDTNTYVGERVE |
| | | LSCEVSEDDANVKWFKNGVELTNEPKSRYRIKVEGKKHTLIIEEAAKNDNA |
| | | TYSVMTTGGQSEAKLSVDLRPLKISLALEDQTVRLGQEIHLKCEISENVEGK |
| | | WYKNGQLIEASDRVKLYHKGRIHRFIIASAAVDDEGEYMFVPDAYNINIPC |
| | | KVHVVDPPKLHLDGLGENNTVTVVAGTKLRLEIPITGEPTPKVMWSKGDK |
| | | WITDSGRIRAETYSDSSCLVIDTAEREDSGPFRITLKNEAGEDSALINIKVVD |
| | | VPDPPQAPNVTEVGEDWCVMTWEPPANDGGSPILGYFIERKKKQSSRWMR |
| | | LNFELCKETTFEPKKMIEGVAYEVRVFAVNAIGTSKPSMPSKSFVPLAVTSP |
| | | PTLLAVDSVTDTSVTMKWRPPDHIGAAGLDGYVVEYCFEGTDEWIVANPE |
| | | LTDKTKFTINGLPTGSKILVRVKAVNAAGESEPRYHPQPILVKEVIEPPKIRL |
| | | PRHLKQTYTRRVGETVNLVIPFQVRTKNTDRKTLNTHFSYATASYLLFLYV |
| | | NLGYWTAFVNILHPEGKSHDKASGIIHSYLGTKIGHYHMSSQSN |
| 170 | Turkey G3URS1 | MNVKGEISAYASLVVKRYKGEIDASHLHAGTFSMPLSLGVTPHGFASRFEI |
| | (Myomesin 1) | KFIDKFEVMFGREGETMSLGCTVVISPDIKRFKPDIEWYRNGVLITPSKWVQ |
| | | MHWTGERASLTLTHANKEDEGLYTLRVVMGEYYEEYSGYVFVRDADAEI |
| | | PGAPGAPLDVQCLDANKDYVIVSWKQPAVDGGNPILGYFIDRCEVGTTHW |
| | | TQCNETPVKFARFPVTGLIEGRSYIFRVRAVNRAGVSLPSRVSEAVAALDPA |
| | | DRARLRSHPSAPWTGQIIVTEEEPAEGVIPGPPTDLQVIEATKNYVVLSWKP |
| | | PGQRGHEGIMYFVEKCIAGTEDWQRVNTEIPVKSPRFAVFDLSEGKSYCFR |
| | | VRCCNSAGIGEPSEATEATVVDDKLDIPKPPGRIMPTRNTDTSVVVTWTEPP |
| | | DAKELVGYYIESSVVGSGHWEPCNNNPVKGTRFICHGLVNGEKYIFRVRAV |
| | | NAAGLSEFSQDSEPIEVQAAIAAPSPPYDITVLESVRDSMVLGWKQPKATGG |
| | | TEITGYYVNYREVIGGVPGKWREANIKAISERAYRIENLKENMVYQFQVAA |
| | | ANLAGVGTPSLPSKPFKCEEWTIAVPGPPHDVTYTEVRKDSLVLLWKEPVY |
| | | TGRSPIAGYYIDMKETEAKEEHWRSVNEKPLQKKFLKIAGLTEGVSYVFRV |
| | | RATNQAGVGRPSDITDPVVAETRPGTKEVVVNVDDDGVISLNFECDQMSP |
| | | NSKFVWSKNYEPIEDDSRLDIDSKGGKSKAIFKNLGEDDLGIYSCVVTDTDG |
| | | VSSGYTIDEEEMKRLLALSHERKFPTIPLASELAVEILEKGEVRFWLQAEKLS |
| | | GNAKANFVFNDKEIFNGEKYKMKVDQRTGLVEMIMDKLEDKDEGTYTFQ |
| | | LQDGKATNQSSLVLIGDVFKKLRDEAEFQRKEWHRKQGPHFVDQLGWEV |
| | | TDECNVMLKCKVANIKKETHIVWYKDDREIMVDEEHDFKDGVCTLLISEFS |
| | | KKDTGIYEVILKDDRGKDVSELKLTDTAFADLMNEVCRRIALSATDLKIQST |
| | | AEGIRLYSFVTYYVDDLRVGWVHNDTQIRYTDRVKTGVTGEQIWLQINEPT |
| | | PQDKGKYTMELFDGKTGHKKTVDLSGQAFDEAFAEFQRLKQAAIAEKNRA |
| | | RVLGGLPDVVTIQEGKALNLSCTVWGDPTPEVSWLKNEKSFVSDANCILKF |
| | | EAGKNVSFTISAVSTHDSGKYSLVVKNKYGTETSDVTVIVITVSGQTDTAK |
| | | HTVSISGQSQNNITVSKKKKATTMSKPQQEKEKKDIKPHVDEIIATEETSDS |
| | | KEIPRVEKEKNTENNQQTAKKTHGKAAAARTVP |
| 171 | Turkey | MSILKIQAREIFDSRGNPTVEVDLYTNKGLFRAAVPSGASTGIYEALELRDN |
| | A0A803YQS2 | DKTRYLGKGVSKAVEHVNKTIAPALISKNVNVVEQEKIDKLMLEMDGTEN |
| | (Phosphopyruvate | KSKFGANAILGVSLAVCKAGAAEKGVPLYRHIADLAGNPEVILPVPAFNVI |
| | hydratase) | NGGSHAGNKLAMQEFMILPVGADNFKEAMRIGAEVYHNLKNVIKEKYGK |
| | | DATNVGDEGGFAPNILENKEALELLKTAIGKAGYSDKVVIGMDVAASEFYR |
| | | DGKYDLDFKSPDDPSRYISPDQLADLYKGFVKNYPLVSIEDPFDQDDWAA |
| | | WKKFTASVGIQVVGDDLTVTNPKRIAKAVEEKSCNCLLLKVNQIGSVTESL |
| | | QACKLAQSNGWGVMVSHRSGETEDTFIADLVVGLCTGQIKTGAPCRSERL |
| | | AKYNQLLRIEEELGSKARFAGRNFRNPRIN |
| 172 | Turkey G1N0W1 | MAPKKAKKRIEGANSNVFSMFEQAQIQEFKEAFTIMDQNRDGFIDKADLRD |
| | (Myosin light | TFAALGRLNVKNEEIDEMIKEAPGPINFTVFLTMFGEKLKGADPEETILNAF |
| | chain 2) | KVFDPEGKGLKSAYIKEMLMTQGERFSQEEIDQMFAAFPPDVSGNLDYKNL |
| | | VHVITHGEEKD |
| 173 | Turkey GIMSQ0 | MSSDAEMAIFGEAAPYLRKSEKERIEAQNRPFDAKAACFVVDQKQMYVKG |
| | (Myosin heavy | TIQSREGGKVTVKTYDDTTVTVKDDEVFPMNPPKYDKIEDMAMMTHLHEP |
| | chain 2) | AVLYNLKERYAAWMIYTYSGLFCVTVNPYKWLPVYNPEVVSGYRGKKRQ |
| | | EAPPHIFSISDNAYQFMLTDRDNQSILITGESGAGKTVNTKRVIQYFATIAVT |
| | | GEKKKDQQPSKMQGTLEDQIISANPLLEAFGNAKTVRNDNSSRFGKFIRIHF |
| | | GTTGKLASADIETYLLEKSRVTFQLASERSYHIFYQIMSNKKPELIGEYLLLI |
| | | TTNPYDFTYVSQGEITVASIDDSEELLATDNAVDILGFSPDEKVGMYKLTGA |
| | | VMHYGNMKFKQKQREEQAEPDGTEVADKAAYLMGLNSAELLKALCYPR |
| | | VKVGNEYVTKGQNVQQVYNSVGALAKSVYEKMFLWMVTRINQQLDTKQ |
| | | PRQHFIGVLDIAGFEIFDFNSLEQLCINFTNEKLQQFFNHHMFVLEQEEYKKE |
| | | GIDWEFIDFGMDLAACIELIEKPMGIFSILEEECMFPKATDTSFKNKLYDQHL |
| | | GKSNNFQKPKPAKGKAEAHFSLIHYAGTVDYNITGWLEKNKDPLNETVVG |
| | | LYQKSSLKLLSFLFSNYAGAETSDSGSGKKGGKKKGGSFQTVSAVFRENLN |
| | | KLMTNLRSTHPHFVRCLIPNETKTPGEMDHYLVMHQLRCNGVLEGIRICRK |
| | | GFPSRILYADFKQRYKILNASAIPEGQFIDSKKASEKLLSSIDVDHNQYRFGH |
| | | TKVFFKAGLLGLLEEMRDEKLVSLITHMQAMCRGYLMRTEFKKMNARRES |
| | | IYIIQYNVRAFMNVKHWPWMKLYFKMKPMLKSVESEKEMANMKEEFEKT |
| | | KEELAKSEAKRKELEEKMVSLLQEKNDLQLQVQSESENLADAEERCEGLIK |
| | | SKIQLEARIKELNERMEDEEEMNAELTAKKRKLEDECSELKKDIDDLELTLA |
| | | KVEKEKHATENKVKNLTEEMAALDENISKLTKEKKALQEAHQQTLEYLQA |
| | | EEDKVSILTKIKTKLEQQVDDLEGSLEQERKQRMDLERAKRKLDGDLKMS |
| | | QDSIMDLKNDKQQTDERLKKKDFEISHLQSKIEDEQAQSSQLQKKIKELQA |
| | | RTEELEEEIEAERANCAKTEKQRADLSRELEEISERLEEAGGATAAQIEMNK |
| | | KREAEFQKMRRDLEEATLQHEATAAALRKKHADSTAELGEQIDNLQRVKQ |
| | | KLEKEKSELKMEIDDLASNMESVSKAKSNLEKMCRALEDQYSEVRTKDDE |
| | | HMRLINDMNTQKTRLQTENGELTRQLEEKESLISQLTRGKQAITQQTEELK |
| | | RQLEEENKAKNALAHGLQSARHDCDLLREQYEEELEAKGELQRALSKANS |
| | | EVAQWRTKYETDAIQCMEELEDAKKKLAQRLQDSEEQIEAVNSKCASLEK |
| | | TKQRLQGEVDDLMIAVERSNAACAAFDKKQKNFDKVMAEWKQKYQESQ |
| | | AELEAAQKESRTLSTEIFRMKNAYEEVLDQVETVRRENKNLQQEISDLTEQ |
| | | LAEAGKANHGLEKAKKQTEQEKYDLQAALEEAEVSLEHEEGKILRVQLEL |
| | | NQVKSGVDRRSAEKDEEIQQLKKNHQRVLESMQTMLDAEIRSRNDALRLK |
| | | KKMEGDLNDMEIQLSRANCQIAEIQKHLKGMQGQLKDSQLHLDDALREND |
| | | DLKEQLAVVERRNNLMMTELEQMRAALEQTERARKVSEQELTDTSERVQI |
| | | LHSQNTSLLNTKKKLEVDITHLQNEVEDSIQEASNAEEKAKKAITDAAMMA |
| | | EELKKEQDTSAHLERMKRNLEQMVKDLQHRLDEAEQLTLKGGKKQLQKL |
| | | EARIHELENELDIEQKRGTESLKGARKYERRLKELTYQSEEDKKNILRLQNL |
| | | VDKLQLKVKAYKKQAEEAEEQASTNLSRCRKTQHELEEAEERADIAECQV |
| | | NKLRAKSRDTGRQKSGLSSELGDNTFLPVIWHNMCNIDRPETTPQVILLHQ |
| 174 | Turkey GINCR2 | MKWVTLISFIFLFSSATSRNLQRVARDAEHKSEIAHRYNDLKEETFKAVAMI |
| | (Albumin) | TFAQYLQRCSYDGLSKLVKDVVDLAQKCVANEDAPECSKSLPSIILDEICQV |
| | | EKLRDSYGAMADCCSKADPERNECFLSFKVSQPDFIQPYQRPASDVICKEY |
| | | QDNRVSLLGHFIYSVARRNPYMYAPTILSFAADYEHALQSCCQESDVGACL |
| | | DAKETAMREKAKKISVKQQYSCGILKKFGDRVFQAEKLVRLSQKYPKAPFS |
| | | EVAKLVRESKDVHKECCEGDMVECMDDMAEIINNMCSKQDVFSSKIKGCC |
| | | EKPIMERSQCIMEAEFDEKPADLPSLVEKYIQDKEVCKSFEAGHDAFMSEFV |
| | | YEYSRRHPEFSTQLILRIAKGYESLLEKCCKTDNPAECYANAQDQLNQHIKE |
| | | TQDVVKTNCDLLSAHGEPDFLKSILIRYTKKMPQVSTETLLEIGKKMTAIGT |
| | | KCCQLSEDRRMACSEGYLSIVIHDMCRRQETTPINDNVSHCCSDSYAHRRP |
| | | CFTAMGVDTKYVPPPFNPDMFSFDEKLCSAPAEEREVGQMKLLINLIKRKP |
| | | QMTEEQIKTIADGFTAMVDKCCKQSDINTCFGEEGANLIVQSRATLGIGV |

An animal protein can comprise a duck protein (Table 10 and Table 11). A duck protein can comprise a duck muscle protein. A duck protein can comprise a duck blood protein. A duck protein can comprise a duck albumin protein. A duck protein can comprise a protein from a mallard duck (Anas platyrhynchos). A duck protein can comprise a protein from a Northern mallard duck (Anas platyrhynchos platyrhynchos). A duck protein can comprise a protein from a Muscovy duck (Cairina moschata). A duck protein can comprise a protein from a domestic Muscovy duck (Cairina moschata domestica). A duck protein can comprise a sequence that has at least or about 70%, at least or about 75%, at least or about 80%, at least or about 90%, at least or about 91%, at least or about 92%, at least or about 93%, at least or about 94%, at least or about 95%, at least or about 96%, at least or about 97% at least or about 98% at least or about 99% or about 100% sequence identity to any one of SEQ ID NO: 175-200.

A food product may comprise a fragment or a truncation of a duck protein. The food product may include a fragment or truncation of any one of SEQ ID Nos: 175-200. A sequence of the fragment or truncation may be identical or substantially identical to a portion of any one of SEQ ID Nos: 175-200. A sequence of the fragment or truncation may be at least about 70%, 80%, 90%, 95%, 98%, or more identical to a portion of a sequence of any one of SEQ ID Nos: 175-200. A length of the fragment or truncation that is at least about 10%, 15%, 20%, 30%, 40%, 50%, 60%, 70%, 80%, 90%, or more of a length of any one of SEQ ID Nos: 175-200. A length of the fragment or truncation may be less than or equal to about 90%, 80%, 70%, 60%, 50%, 40%, 30%, 20%, 15%, 10%, or less than a length of any one of SEQ ID Nos: 175-200.

TABLE 10
Northern Mallard Duck Proteins

| SEQ | | |
|---|---|---|
| ID NO | Description | Amino Acid Sequence |
| 175 | Northern Mallard | MPFSNSHNLLKMKHSATDEFPDLSAHNNHMAKVLTLDLYKKLRDKQTPSG |
| | Duck | FTLDDVIQTGVDNPGHPFIMTVGCVAGDEESYEVFKELFDPVIEDRHGGYK |
| | A0A493TFK1 | PTDQHKTDLNAGNLQGGDDLDPNYVLSSRVRTGRSIRGFCLPPHCSRGERR |
| | (Creatine kinase) | AIEKLSVEALGSLGGDLKGKYYALRNMTDAEQQQLIDDHFLFDKPVSPLLL |
| | | ASGMARDWPDARGIWHNDNKTFLVWINEEDHLRVISMQKGGNMKEVFTR |
| | | FCTGLTQIENLFKSKNHEFMWNPHLGYILTCPSNLGTGLRAGVHIKLPHLGK |
| | | HEKFGEVLKKLRLQKRGTGGVDTAAVGGVFDVSNADRLGFSEVELVQMV |
| | | VDGVKLLIEMEKRLEKGQSIDDLIPAQK |
| 176 | Northern Mallard | MNSMNQIETNMQYTYNYEEDEYMTQEEEWDRDLLLDPAWEKQQRKTFTA |
| | Duck | WCNSHLRKAGTQIENIEEDFRNGLKLMLLLEVISGERLPKPDRGKMRFHKI |
| | A0A493TCE5 | ANVNKALDYIASKGVKLVSIGAEEIVDGNVKMTLGMIWTIILRFAIQDISVE |
| | (Actinin alpha 2) | ETSAKEGLLLWCQRKTAPYRNVNIQNFHLSWKDGLGLCALIHRHRPDLIDY |
| | | SKLNKDDPIGNINLAMEIAEKHLDIPKMLDAEDIVNTPKPDERAIMTYVSCF |
| | | YHAFAGAEQAETAANRICKVLAVNQENERLMEEYERLASELLEWIRRTIPW |
| | | LENRTPEKTMQAMQKKLEDFRDYRRKHKPPKVQEKCQLEINFNTLQTKLRI |
| | | SNRPAFMPSEGKMVSDIAGAWQRLEQAEKGYEEWLLNEIRRLERLEHLAE |
| | | KFRQKASTHEQWAYGKEQILLQKDYESASLTEVRAMLRKHEAFESDLAAH |
| | | QDRVEQIAAIAQELNELDYHDAASVNDRCQKICDQWDSLGTLTQKRREAL |
| | | ERTEKLLETIDQLHLEFAKRAAPFNNWMEGAMEDLQDMFIVHSIEEIQSLIS |
| | | AHDQFKATLPEADGERQAILSIQNEVEKVIQSYSMRISASNPYSTVTVEEIRT |
| | | KWEKVKQLVPQRDQSLQEELARQHANERLRRQFAAQANVIGPWIQTKME |
| | | EIARSSIEMTGPLEDQMNQLKQYEQNIINYKHNIDKLEGDHQLIQEALVEDN |
| | | KHTNYTMEHIRVGWELLLTTIARTINEVETQILTRDAKGITQEQMNDFRASF |
| | | NHFDRRKNGLMDHDDFRACLISMGYDLGEAEFARIMSLVDPNGQGTVTFQ |
| | | SFIDFMTRETADTDTAEQVIASFRILASDKPYILADELRRELPPEQAQYCIKR |
| | | MPPYTGPGSVPGALDYTSFSSALYGESDL |
| 177 | Northern Mallard | MAAPLSDGEKRKQISVRGIAGLGDVAEVRKSFNRHLHFTLVKDRNVATPR |
| | Duck U3ISG6 | DYFFALAHTVRDHLVGRWIRTQQHYYERDPKRIYYLSLEFYMGRTLQNTM |
| | (Alpha-1,4 glucan | VNLGLQNACDEAIYQLGLDLEELEEIEEDAGLGNGGLGRLAACFLDSMATL |
| | phosphorylase) | GLAAYGYGIRYEFGIFNQKIVDGWQVEEADDWLRYGNPWEKARPEYMLP |
| | | VHFYGRVEHTPEGVKWVDTQVVLAMPYDTPVPGYKNNTVNTMRLWSAK |
| | | APNDFNLQEFNVGDYIEAVLDRNLAENISRVLYPNDNFFEGKELRLKQEYF |
| | | VVAATLQDIIRRFKSSKFGCRDPVRTSFETFPDKVAIQLNDTHPALSIPELMR |
| | | ILVDVEKVDWDKAWEITKRTCAYTNHTVLPEALERWPVSMFEKLLPRHLEI |
| | | IYALNQMHLDRVAALYPGDVDRLRRMSVIEEGDCKRINMAHLCVIGSHAV |
| | | NGVARIHSDIVKHSVFKDFYELEPEKFQNKTNGITPRRWLLLCNPGLADVIA |
| | | EKIGEGFVTDLSQLKKLLDFIDNEMFIRDVAKVKQENKLKFSAYLEEQYKV |
| | | KINPSSMFDVQVKRIHEYKRQLLNCLHAITLYNRIRSDPSKSFVPRTIMIGGK |
| | | AAPGYHMAKMIIKLITSIGEVINNDPYVGDKLKVIFLENYRVSLAEKVIPAA |
| | | DLSQQISTAGTEASGTGNMKFMLNGALTIGTMDGANVEMAEEAGEENLFIF |
| | | GMRVEDVEALDRKGYNALEYYERLPELRQAIDQISSGFFSPRDPGCFRDVV |
| | | NMLMHHDRFKVFADYEAYIKCQGQVDQLFMDPREWTKKVIRNIACSGKFS |
| | | SDRTITEYAQEIWGVEPSSTKIPPPNIPRD |
| 178 | Northern Mallard | MTHQFPALSPEQKKALSDIAQRIVASGKGILAADESVGTMGNRLQRINVEN |
| | Duck U3IHG8 | TEENRRAFREILFSSDTSINQSIGGVILFHETLYQKDSSGKPFPAIIKEKGIVVG |
| | (Fructose- | IKLDKGTSPLAGTNGETTIEGLDGLSERCAQYKKDGVDFGKWRAVLKITST |
| | bisphosphate | TPSQLAIQENANTLARYASICQQNGLVPIVEPEVLPDGDHDLQRCQYVTEK |
| | aldolase) | VLAAVYKALNDHHVYLEGTLLKPNMVTAGHACTKKYTPQDVAAATVTTL |
| | | LRTVPAAVPGICFLSGGQSEEEASLNLNAMNQSSLPKPWKLTFSYGRALQA |
| | | SALAAWLGKSENKKAAQEAFRKRAQINSLACRGQYVVSGKTDAAATQSLF |
| | | TASYTY |
| 179 | Northern Mallard | MAPKKDVKKPAAAAAPAPAPAPAPAPAPAKPKEPAIDLKSIKIEFSKEQQD |
| | Duck U3IA79 | DFKEAFLLFDRTGDAKITLSQVGDIVRALGQNPTNAEINKILGNPSKEEMNA |
| | (Myosin light | KKITFEEFLPMLQAAANNKDQGTYEDFVEGLRVFDKEGNGTVMGAELRHV |
| | chain 1) | LATLGEKMTEEEVDELMKGQEDSNGCINYEAFVKHILSV |
| 180 | Northern Mallard | MVKVGVNGFGRIGRLVTRAAILSGKVQVVAINDPFIDLNYMVYMFKYDST |
| | Duck | HGHFRGTVKAENGKLVINGNAITIFQERDPSNIKWADAGAEYVVESTGVFT |
| | A0A493TQC9 | TMEKAGAHLKGGAKRVIISAPSADAPMFVMGVNHEKYDKSLKIVSNASCT |
| | (Glyceraldehyde- | TNCLAPLAKVIHDNFGIVEGLMTTVHAITATQKTVDGPSGKLWRDGRGAA |
| | 3-phosphate | QNIIPASTGAAKAVGKVIPELNGKLTGMAFRVPTPNVSVVDLTCRLEKPAK |
| | dehydrogenase) | YDDIKRVVKAAADGPLKGILAYTEDQVVSCDFNGDSHSSTFDAGAGIALND |
| | | HFVKLVSWYDNEYGYSNRVLALSTLLHAANIFPSSR |
| 181 | Northern Mallard | MEAIKKKMQMLKLDKENALDRAEQAEAEQKQAEERSKQLEDELAAMQK |
| | Duck | KLKGTEDELDKYSEALKDAQEKLELAEKKAADAEAEVASLNRRIQLVEEE |
| | A0A493TRM1 | LDRAQERLATALQKLEEAEKAADESERGMKVIENRALKDEEKMELQEIQL |
| | (Tropomyosin 3) | KEAKHIAEEADRKYEEVARKLVIIEGDLERTEERAELAESKCSELEEELKNV |
| | | TNNLKSLEAQAEKYSQKEDKYEEEIKILTDKLKEAETRAEFAERSVAKLEK |
| | | TIDDLEDELYAQKLKYKAISEELDHALNDMTSI |
| 182 | Northern Mallard | MPEPVKKTVSAFTKKPKTTEVTAGSTAVFEAETEKASVKVKWQRAGTEIT |
| | Duck U3J9W2 | DSEKYVIKAEGNKHSLTINNVVKEDDVTYAVIAGSSKVKFELKVKEPEKSE |
| | (Myosin binding | TVTPAEAAPAPAASELPAPPAESSQNTEVPSAGTQPEEPADPIGLFVTRPQDG |
| | protein C3) | EVTVGGNITFTAKVAGESLLKKPSVKWFKGKWMDLASKVGQHLQLHDSY |
| | | DRNNKVYTFELEIIEANMTFAGGYRCEVSTKDKFDSSNFNLTVNEAPLSGD |
| | | LDIRAAFRRTTEGHDEAGELNFSALLKKRGDGKTESQPDVDVWEILRKAPP |
| | | SEYEKIAFQYGITDLRGMLKRLKRIKKEEKKSTAFLKKLDPAYQVDKGQKI |
| | | KLMVEVANPDADVKWLKNGQEIQVSGSRYIFEAVGNKRILTINHCSLADD |
| | | AAYECVVGEEKSFTELFVKEPPILITRPLEDQMVMVGERVEFECEVSEEGAT |
| | | VKWEKDGVELTREETFKYRFKKDGKKQYLIINESTKEDSGHYTVKTNGGV |
| | | SVAELIVQEKKLEVYQSIADLTVKARDQAVFKCEVSDENVKGIWLKNGKE |
| | | VVPDERIKISHIGRIHKLTIEDVTPGDEADYSFIPQGFAYNLSAKLQFLEVKID |
| | | FVPREEPPKIHLDCLGQSPDTIVVVAGNKLRLDVPISGDPTPTVIWQKVNKK |
| | | TDLLRSNDDSMTPSENSSDLNTDSKLLFESEGRVRVEKHEDHCVFIIEGAEK |
| | | EDEGVYRVIVKNPVGEDKADITVKVIDVPDPPEAPKISNIGEDYCTVQWQPP |
| | | KYDGGQPVLGYILERKKKKSYRWMRLNFDLLKELTYEAKRMIEGVVYEM |
| | | RIYAVNSIGMSRPSPASQPFMPIAPPSEPTHFTVEDVSDSTVALKWRPPERIG |
| | | AGGLDGYIVEYCKEGSTEWIPALPGLTERTSALIKDLVTGDKLHFRIKAINL |
| | | AGESGAAIIKEPITVQEILQRPKIWLPRHLRQTLVKKVGETINIVIPFQGKPRP |
| | | KITWTKDGQSLDAKDVGIRNSNTDTILFIRKAELHHSGAYEITLQIENMTDK |
| | | VAITIQIIDKPGPPQNIKLTDVWGFNAALEWTPPQDDGNAQILGYTVQKAD |
| | | KKTMEWYTVFDHYRRTNCVVSELIMGNEYFFRVFSENLCGLSETAATTKN |
| | | PAYIQKTGTTYKPPCYKEHDFSEPPKFTHPLVNRSVIAGYNTTLSCAVRGIP |
| | | KPKIYWYKNKVDLSGDAKYRMFSKQGVLTLEIRKPTPFDGGFYTCKAVNE |
| | | CGEAEIECKLDVRVPQ |
| 183 | Northern Mallard | MSSLAVPFYQRRHKHFDQSYRNIQTRYVLEEYAARKAASRQAAYYESSGL |
| | Duck | GKTTCRLCARRARTLAHETMQESRKRTHEQKSHASEEKRIRFASELSALEK |
| | A0A493TRW8 | EIHTARHRAREQLDRLAIQRMVEENMAMERHAVEERISRAPEILVRLRSHT |
| | (Myomesin 2) | VWEKMSVSLCFTVQGFPTPVVQWYKNEELITPASDPAKYRVESKYGVHVL |
| | | HINRADFDDSATYSAVATNVHGQASTNCAVVVRRFREGEEPHPAGIMPFHL |
| | | PLSYDVCFTHFDVQFLEKFGVTFATEGETLTLKCSVLVTPELKRLRPRAEW |
| | | YRDDVLIKDSKWTKLYFGEGQAALSFTHLNKDDEGLYTLRMITKGGVNEC |
| | | SAYLFVRDADALIAGAPGAPMDVKCHDANRDYVIVTWKPPNTTSQSPVIG |
| | | YFVDKCEVGLENWVQCNDAPVKICKYPVTGLYEGRSYIFRVRAVNNAGIS |
| | | RPSRVSEPVAALDPVDLERTQTIHVDEGRKIVISKDDLEGDIQIPGPPTNVHA |
| | | SEISKTYVVLSWDPPVPRGREPLTYFIEKSMVGSGSWQRVNAQVAVKSPRY |
| | | AVFDLAEGKPYVFRVVSANKHGISDPSEITEPIQPQDTVVVPSAPGRVVATR |
| | | NTKTSVVVQWDKPKHEEDLYGYYIDYSVVGSNHWEPSNHKPIKYNRFVVH |
| | | GLETGEQYIFRVKAVNAVGFSENSQESEAIKVQAALTCPSYPHGITLLNCDG |
| | | HSMTLGWKAPKYSGGSPILGYYMDKREANHQNWHEVNSSLITRTIYTVED |
| | | LTEDAFYEFKIAAANIVGIGQPSDPSEHFKCKAWTMPEPGPAYDLTVCEVR |
| | | NTSLVLLWKAPVYEGKSPITGYLVDYKEVDKEDWITANEKPTAQRYFKVT |
| | | DLHEGHTYVFKVRAVNDAGVGKSSEVSEPVLVEAKPGTKEIFSGVDDEGNI |
| | | YLAFECKEATDASHFAWGKSYEEIEDSDKYKIETEGDHSKLYFKNPDKSDL |
| | | GTYSISVSDTDGVSSSFVMDDEELERLMAISNEIKNPTIPLKSELAYEVLEKG |
| | | EVRFWIQAESLSPDSTFRFVINDKEVENSDRHKINCDRPNGIIEMVMDRFTID |
| | | NEGTYTVQIQDGKAKNQSSLVLIGDAFKAVLAESELQRKEFLRKQGPHFSE |
| | | FLYWEVTEECEVLLACKVANTKKETVFKWYKDGSGIDVDEMPDLQKGEC |
| | | HLSIPRLSRKDEGVYKATLSDDRGHDVSTLELSGKVYDDIILALSRVSGKTA |
| | | SPLKILCTEEGIRLQCFLKYYNEEMKISWSHRESKISSGEKMKIGGGENIAWL |
| | | QITEPTEKDKGNYTFEIFGDKESFKRTLDLSGQAFDDAYAEFQRLKAAAFAE |
| | | KNRGKVIGGLPDVVTIMDGKTLNLTCTVFGNPDPEVVWFKNDKALELNEH |
| | | YVVSLEQGKYASLTIKGVTSEDSGKFSIHVKNKYGGETVDVTVSVYRHGEK |
| | | VPEVKPGQLAKPRPIPPSEDSS |
| 184 | Northern Mallard | MPERSWIPVGTPLLRWTCTHTKACFEQRSPAVPPLVSTKHWSSETTTSPVSL |
| | Duck | EKGLSVVDQEKIDNLMLEMDGTENKSKFGANAILGVSLAVCKAGAAEKDV |
| | A0A493T6U6 | PLYRHIADLAGNSDLILPVPAFNVINGGSHAGNKLAMQEFMILPVGAESFRD |
| | (Phosphopyruvate | AMRIGAEVYHNLKSVIKEKYGKDATNVGDEGGFAPNILENSEALELLKEAI |
| | hydratase) | DKAGYTDKIVIGMDVAASEFYRDGKYDLDFKSPDDPSRYISADELGDLYQS |
| | | FVRDYPVVSIEDPFDQDDWEAWSKFTANVGIQIVGDDLTVTNPKRIERAVE |
| | | EKACNCLLLKVNQIGSVTEAIQACKLAQENGWGVMVSHRSGETEDTFIADL |
| | | VVGLCTGQIKTGAPCRSERLAKYNQLMRIEEELGDEARFAGHNFRNPSVL |
| 185 | Northern Mallard | MQGENHRAPKKAKKRIEGANSNVFSMFEQAQIQEFKEAFTIMDQNRDGFID |
| | Duck U3I9B1 | KADLRDTFAALGRLNVKNEEIDEMIKEAPGPINFTVFLTMFGEKLKGADPE |
| | (Myosin light | ETILNAFKVFDPEGKGLKSAYIKEMLMTQGERFSQEEVDQMFAAFPPDVSG |
| | chain 2) | NLDYKNLVHVITHGEEKD |
| 186 | Northern Mallard | FSISDNAYQFMLTDRENQSILITGESGAGKTVNTKRVIQYFATIAASGEKKK |
| | Duck U3J7T0 | EEQSGKMQGTLEDQIISANPLLEAFGNAKTVRNDNSSRFGKFIRIHFGATGK |
| | (Myosin motor | LASADIETYLLEKSRVTFQLKAERSYHIFYQIMSNKKPELIDMLLITTNPYDY |
| | domain- | HFVSQGEITVASINDQEELMATDSAIDILGFTADEKVAIYKLTGAVMHYGN |
| | containing | LKFKQKQREEQAEPDGTEVADKAAYLMGLNSADLLKALCYPRVKVGNEY |
| | protein) | VTKGQTVQQVNNSVGALAKAVYEKMFLWMVVRINQQLDTKQPRQYFIGV |
| | | LDIAGFEIFDFNSLEQLCINFTNEKLQQFFNHHMFVLEQEEYKKEGIEWEFID |
| | | FGMDLAACIELIEKPMGIFSILEEECMFPKATDTSFKNKLYDQHLGKSSNFQ |
| | | KPKPAKGKVEAHFSLVHYAGTVDYNITGWLEKNKDPLNETVIGLYQKSSV |
| | | KTLALLFANYGGAEINPQKIIIKDTFKGSSFQTVSALFRENLNKLMTNLRSTH |
| | | PHFVRCIIPNETKTPGAMEHELVLHQLRCNGVLEGIRICRKGFPSRVLYADF |
| | | KQRYKVLNASAIPEGQFIDSKKASEKLLGSIDVDHTQYKFGHTKVFFKAGL |
| | | LGLLEEMRDEKLAQLITRTQARCRGFLMRVEYQRMVERRESIFCIQYNVRA |
| | | FMNVKHWPWMKLFFKIKPLLKSAESEKEMANMKEEFEKTKEELAKSEAK |
| | | KKELEEKMVKLVQEKNDLQLQVQAEADALADAEERCDQLIKTKIQLEAKI |
| | | KEVTERAEDEEEINAELTAKKRKLEDECSELKKDIDDLELTLAKVEKEKHA |
| | | TENKVKNLTEEMAALDETIAKLTKEKKALQEAHQQTLDDLQAEEDKVNTL |
| | | TKAKTKLEQQVDDLEGSLEQEKKLRMDLERAKRKLEGDLKLSQDSIMDLE |
| | | NDKQQLDEKLKKKDFEISQIQSKIEDEQALGMQLQKKIKELQARIEELEEEIE |
| | | AERTSRAKAEKHRADLSRELEEISERLEEAGGATAAQIEMNKKREAEFQKM |
| | | RRDLEEATLQHEATAAALRKKHADSTAELGEQIDNLQRVKQKLEKEKSEL |
| | | KMEIDDLASNMESVSKAKANLEKMCRTLEDQLSEIKTKEEEHQRMINDLTA |
| | | QRARLQTESGEYSRQVEEKDSQISQLSRSKQAFTQQIEELKRHLEEEIKAKN |
| | | ALAHALQSARHDCDLLREQYEEEQEAKGELQRALSKANSEVAQWRTKYE |
| | | TDAIQRTEELEEAKKKLAQRLQDAEEHVEAVNAKCASLEKTKQRLQNEVE |
| | | DLMIDVERSNAACAALDKKQKNEDKILAEWKQKYEETQAELEASQKESRS |
| | | LSTELFKMKNAYEESLDHLETLKRENKNLQQEISDLTEQIAEGGKAIHELEK |
| | | VKKQIEQEKSEIQAALEEAEASLEHEEGKILRLQLELNQVKSEIDRKIAEKDE |
| | | EIDQLKRNHLRIVESMQSTLDAEIRSRNEALRLKKKMEGDLNEMEIQLSHA |
| | | NRQAAEAQKNLRNTQGVLKDTQIHLDDALRTQEDLKEQVAMVERRANLL |
| | | QAEIEELRAALEQTERSRKVAEQELMDASERVQLLHTQNTSLINTKKKLET |
| | | DITQIQSEMEDAIQEARNAEEKAKKAITDASMMAEELKKEQDTSAH |
| 187 | Northern Mallard | MITFAQYLQRCSYDGLSKLVKDVVDLAQKCVANEDAPECSKSLPSIFLDEIC |
| | Duck U3IU92 | QVEKLRDSYGAMADCCAKSDPERNECFLSFKIPQPDFVQPYQRPASDVICK |
| | (Albumin) | EYEDNRVSFLGHFIYSVARRNPFLYAPTILGVAADYEHALKSCCKESDVGA |
| | | CLDGKETGIREKVKKISVKQQYSCGILKKFGDRIFQADKLALLSQKYPKASF |
| | | AEISKLIHDVKDVYKECCEGDMVECMDDRAELVNYMCSKHEAFSSKIKDC |
| | | CEKPIVERSQCIMEAEFDEKPADLPSLVEKYIQDKEVCKSFEAGHDAFMSEF |
| | | VYEYSRRHPEFSTQLILRIAKGYETLLEKCCKTDNPAECYANAQDQLNEHIK |
| | | ETQDVVKTNCDLLTTHGEADFLKALLIRYTKKMPQVSTETLLEIGKKMTGV |
| | | GTKCCKLPEDRRLPCSEGYLSMVIQDMCRRQETSPINDNVSHCCSDSYAYR |
| | | RPCFSAMGVDTKYVPPPFNPDVFNFDEKLCSAPAEERAEGQMKLLINLIKR |
| | | KPQMTEEQIKTIADGFTAMVDKCCKQSDIETCFGEEGANLIVQSRATLGIGA |

TABLE 11
Domestic Muscovy Duck Proteins

| SEQ | | |
|---|---|---|
| ID NO | Description | Amino Acid Sequence |
| 188 | Domestic | MCCLKCQQGFWDFCAPAVLARGPCFGCGALVTAVGSAMPFSNSHNLLKM |
| | Muscovy Duck | KHSATDEFPDLSAHNNHMAKVLTLDLYKKLRDKQTPSGFTLDDVIQTGVD |
| | A0A8C3BBL6 | NPGHPFIMTVGCVAGDEESYEVFKELFDPVIEDRHGGYKPTDQHKTDLNAG |
| | (Creatine kinase) | NLQGGDDLDPNYVLSSRVRTGRSIRGFCLPPHCSRGERRAIEKLSVEALGSL |
| | | GGDLKGKYYALRNMTDAEQQQLIDDHFLFDKPVSPLLLASGMARDWPDA |
| | | RGIWHNDNKTFLVWINEEDHLRVISMQKGGNMKEVFTRFCTGLTQIENLFK |
| | | SKNHEFMWNPHLGYILTCPSNLGTGLRAGVHIKLPHLGKHEKFGEVLKKLR |
| | | LQKRGTGGVDTAAVGGVFDVSNADRLGFSEVELVQMVVDGVKLLIEMEK |
| | | RLEKGQSIDDLIPAQK |
| 189 | Domestic | RAGDKPQDEYMTQEEEWDRDLLLDPAWEKQQRKTFTAWCNSHLRKAGT |
| | Muscovy Duck | QIENIEEDFRNGLKLMLLLEVISGERLPKPDRGKMRFHKIANVNKALDYIAS |
| | A0A8C3BEA7 | KGVKLVSIGAEEIVDGNVKMTLGMIWTIILRFAIQDISVEETSAKEGLLLWC |
| | (Actinin alpha 2) | QRKTAPYRNVNIQNFHLSWKDGLGLCALIHRHRPDLIDYSKLNKDDPIGNI |
| | | NLAMEIAEKHLDIPKMLDAEDIVNTPKPDERAIMTYVSCFYHAFAGAEQAE |
| | | TAANRICKVLAVNQENERLMEEYERLASELLEWIRRTIPWLENRTPEKTMQ |
| | | AMQKKLEDFRDYRRKHKPPKVQEKCQLEINFNTLQTKLRISNRPAFMPSEG |
| | | KMVSDIAGAWQRLEQAEKGYEEWLLNEIRRLERLEHLAEKFRQKASTHEQ |
| | | WAYGKEQILLQKDYESASLTEVRAMLRKHEAFESDLAAHQDRVEQIAAIA |
| | | QELNELDYHDAASVNDRCQKICDQWDSLGTLTQKRREALERTEKLLETIDQ |
| | | LHLEFAKRAAPFNNWMEGAMEDLQDMFIVHSIEEIQSLITAHDQFKATLPE |
| | | ADGERQAILSIQNEVEKVIQSYSMRISASNPYSTVTVEEIRTKWEKVKQLVP |
| | | QRDQSLQEELARQHANERLRRQFAAQANVIGPWIQTKMEEIARSSIEMTGP |
| | | LEDQMNQLKQYEQNIINYKHNIDKLEGDHQLIQEALVFDNKHTNYTMEHIR |
| | | VGWELLLTTIARTINEVETQILTRDAKGITQEQMNDFRASFNHFDRRKNGL |
| | | MDHDDFRACLISMGYDLGEAEFARIMSLVDPNGQGTVTFQSFIDFMTRETA |
| | | DTDTAEQVIASFRILASDKPYILADELRRELPPEQAQYCIKRMPPYTGPGSVP |
| | | GALDYTSFSSALYGESDL |
| 190 | Domestic | MAAPLSDGEKRKQISVRGIAGLGDVAEVRKSFNRHLHFTLVKDRNVATPR |
| | Muscovy Duck | DYFFALAHTVRDHLVGRWIRTQQHYYERDPKRIYYLSLEFYMGRTLQNTM |
| | A0A8C3BW27 | VNLGLQNACDEAIYQLGLDLEELEEIEEDAGLGNGGLGRLAACFLDSMATL |
| | (Alpha-1,4 glucan | GLAAYGYGIRYEFGIFNQKIVDGWQVEEADDWLRYGNPWEKARPEYMLP |
| | phosphorylase) | VHFYGRVEHNPEGVKWVDTQVVLAMPYDTPVPGYKNNTVNTMRLWSAK |
| | | APNDFNLQEFNVGDYIEAVLDRNLAENISRVLYPNDNFFEGKELRLKQEYF |
| | | VVAATLQDIIRRFKSSKFGCRDPVRTSFETFPDKVAIQLNDTHPALSIPELMR |
| | | ILVDVEKVDWDKAWEITKRTCAYTNHTVLPEALERWPVSMFEKLLPRHLEI |
| | | IYALNQMHLDRVAALYPGDVDRLRRMSVIEEGDCKRINMAHLCVIGSHAV |
| | | NGVARIHSDIVKHSVFKDFYELEPEKFQNKTNGITPRRWLLLCNPGLADVIA |
| | | EKIGEGFVTDLSQLKKLLDFIDNKMFIRDVAKVKQENKLKFSAYLEEQYKV |
| | | KINPSSMFDVQVKRIHEYKRQLLNCLHAITLYNRIRSDPSKSFVPRTIMIGGK |
| | | AAPGYHMAKMIIKLITSIGEVINNDPYVGDKLKVIFLENYRVSLAEKVIPAA |
| | | DLSQQISTAGTEASGTGNMKFMLNGALTIGTMDGANVEMAEEAGEENLFIF |
| | | GMRVEDVEALDRKGYNALEYYERLPELRQAIDQISSGFFSPGDPGCFRDVV |
| | | NMLMHHDRFKVFADYEAYIKCQGQVDQLFMDPWEWTKKVIRNIACSGKF |
| | | SSDRTITEYAQEIWGVEPSSTKIPPPNIPRD |
| 191 | Domestic | MTHQFPALSPEQKKALSDIAQRIVASGKGILAADESVGTMGNRLQRINVEN |
| | Muscovy Duck | TEENRRAFREILFSSDTSINQSIGGVILFHETLYQKDSSGKPFPAIIKEKGIVVG |
| | A0A8C3CD90 | IKLDKGTSPLAGTNGETTIEGLDGLSERCAQYKKDGVDFGKWRAVLKITST |
| | (Fructose- | TPSQLAIQENANTLARYASICQQNGLVPIVEPEVLPDGDHDLQRCQYVTEK |
| | bisphosphate | VLAAVYKALNDHHVYLEGTLLKPNMVTAGHACTKKYTPQDVAAATVTTL |
| | aldolase) | LRTVPAAVPGICFLSGGQSEEEASLNLNAMNQSSLPKPWKLTFSYGRALQA |
| | | SALAAWLGKPENKKAAQEAFRKRAQINSLACRGQYVVSGKTDAAATQSLF |
| | | TASYTY |
| 192 | Domestic | MVKVGVNGFGRIGRLVTRAAILSGKVQVVAINDPFIDLNYMVYMFKYDST |
| | Muscovy Duck | HGHFRGTVKAENGKLVINGNAITIFQERDPSNIKWADAGAEYVVESTGVFT |
| | A0A8C3CJK8 | TMEKAGAHLKGGAKRVIISAPSADAPMFVMGVNHEKYDKSLKIVSNASCT |
| | (Glyceraldehyde- | TNCLAPLAKVIHDNFGIVEGLMTTVHAITATQKTVDGPSGKLWRDGRGAA |
| | 3-phosphate | QNIIPASTGAAKAVGKVIPELNGKLTGMAFRVPTPNVSVVDLTCRLEKPAK |
| | dehydrogenase) | YDDIKRVVKAAADGPLKGILAYTEDQVVSCDFNGDSHSSTFDAGAGIALND |
| | | HFVKLVSWYDNEYGYSNRVVDLMVHMASKE |
| 193 | Domestic | MDAIKKKMQMLKLDKENALDRAEQAEADKKAAEERSKQLEDELVALQK |
| | Muscovy Duck | KLKGTEDELDKYSESLKDAQEKLELADKKATDAESEVASLNRRIQLVEEEL |
| | A0A8C3GR18 | DRAQERLATALQKLEEAEKAADESERGMKVIENRAQKDEEKMEIQEIQLKE |
| | (Tropomyosin 1) | AKHIAEEADRKYEEVARKLVIIESDLERAEERAELSESKCAELEEELKTVTN |
| | | NLKSLEAQAEKYSQKEDKYEEEIKVLTDKLKEAETRAEFAERSVTKLEKSID |
| | | DLEDELYAQKLKYKAISEELDHALNDMTSM |
| 194 | Domestic | YPTTKPKTTEVTAGSTAVFEAETEKAGVKVKWQRAGTEITDSEKYVIKAEG |
| | Muscovy Duck | NKHSLTINNVVKEDDVTYAVIAGSSKVKFELKVKEPEKSETVTPAEAAPAP |
| | A0A8C3CSW2 | AASELPAPPAESSQNTEVPSAGTQPEEPVDPIGLFVTRPQDGEVTVGGNITFT |
| | (Myosin binding | AKVAGESLLKKPSVKWFKGKWMDLASKVGQHLQLHDSYDRNNKVYTFE |
| | protein C3) | LEIIEANMTFAGGYRCEVSTKDKFDSSNFNLTVNEIYHLCHDIVSFPFPSKTN |
| | | YIFLLLRSTEGHDEAGELNFSALLKKRGDGKTESQPDVDVWEILRKAPPSEY |
| | | EKIAFQYGITDLRGMLKRLKRIKKEEKKSTAFLKKLDPAYQVDKGQKIKLM |
| | | VEVANPDADVKWLKNGQEIQVSGRYIFEAVGNKRILTINHCSLADDAAYEC |
| | | VVGEEKSFTELFVKEPPILITHPLEDQMVMVGERVEFECEVSEEGATVKWE |
| | | KDGVELTREETFKYRFKKDGKKQYLIINESTKEDSGHYTVKTNGGVSVAEL |
| | | IVQEKKLEVYQSIADLTVKARDQAVFKCEVSDENVKGIWLKNGKEVVPDE |
| | | RIKISHIGRIHKLTIEDVTPGDEADYSFIPQGFAYNLSAKLQFLEVKIDFVPRE |
| | | EPPKIHLDCLGQSPDTIVVVAGNKLRLDVPISGDPTPTVIWQKVNKKTDLLR |
| | | SNDDSMTPSENSSDLNTDSKLLFESEGRVRVEKHEDHCVFIIEGAEKEDEGV |
| | | YRVIVKNPVGEDKADITVKVIDVPDPPEAPKISNIGEDYCTVQWQPPKYDG |
| | | GQPVLGYILERKKKKSYRWMRLNFDLLKELTYEAKRMIEGVVYEMRIYAV |
| | | NSIGMSRPSPASQPFMPIAPPSEPTHFTVEDVSDSTVALKWRPPERIGAGGLD |
| | | GYIVEYCKEGSTEWIPALPGLTERTSALIKDLVTGDKLHFRIKAINLAGESGA |
| | | AIIKEPITVQEILQRPKIWLPRHLRQTLVKKVGETINIVIPFQGKPRPKITWTK |
| | | DGQSLDAKDVGIRNSNTDTILFIRKAELHHSGAYEITLQIENMTDKVAITIQII |
| | | DKPGPPQNIKLTDVWGFNAALEWTPPQDDGNAQILGYTVQKADKKTMEW |
| | | YTVFDHYRRTNCVVSELIMGNEYFFRVFSENLCGLSETAATTKNPAYIQKT |
| | | GTTYKPPCYKEHDFSEPPKFTHPLVNRSVIAGYNTTLSCAVRGIPKPKIYWY |
| | | KNKMDLSGDAKYRMFSKQGVLTLEIRKPTPFDGGFYTCKAVNERGEAEIEC |
| | | KLDVRGNHMSIMHNTNTAVK |
| 195 | Domestic | MSSLAVPFYQRRHKHFDQSYRNIQTRYVLEEYAARKAASRQAAYYESSGL |
| | Muscovy Duck | GKTTCRLCARRARTLAHETMQESRKRTHEQKSHASEEKRIRFASELSALEK |
| | A0A8C3CXN6 | EIHTARHRAREQLDRLAIQRMVEENMAMERHAVEERISRAPEILVRLRSHT |
| | (Myomesin 2) | VWEKMSVSLCFTVQGFPTPVVQWYKNEELITPASDPAKYRVESKYGVHVL |
| | | HINRADFDDSATYSAVATNVHGQASTNCAVVVRRFREGEEPHPAGIMPFHL |
| | | PLSYDVCFTHFDVQFLEKFGVTFATEGETLTLKCSVLVTPELKRLRPRAEW |
| | | YRDDVLIKDSKWTKLYFGEGQAALSFTHLNKDDEGLYTLRMITKGGVNEC |
| | | SAYLFVRDADALIAGAPGAPMDVKCHDANRDYVIVTWKPPNTTSQSPVIG |
| | | YFVDKCEVGLENWVQCNDAPVKICKYPVTGLYEGRSYIFRVRAVNNAGIS |
| | | RPSRVSEPVAALDPVDLERTQTIHVDEGRKIVISKDDLEGDIQIPGPPTNVHA |
| | | SEISKTYVVLSWDPPVPRGREPLTYFIEKSMVGSGSWQRVNAQVAVKSPRY |
| | | AVFDLAEGKPYVFRVVSANKHGISDPSEITEPIQPQDTVVVPSAPGRVVATR |
| | | NTKTSVVVQWDKPKHEEDLYGYYIDYSVVGSNHWEPSNHKPIKYNRFVVH |
| | | GLETGEQYIFRVKAVNAVGFSENSQESEAIKVQAALTCPSYPHGITLLNCDG |
| | | HSMTLGWKAPKYSGGSPILGYYMDKREANHQNWHEVNSSLITRTIYTVED |
| | | LTEDAFYEFKIAAANIVGIGQPSDPSEHFKCKAWTMPEPGPAYDLTVCEVR |
| | | NTSLVLLWKAPVYEGKSPITGYLVDYKEVDKEDWITANEKPTAQRYFKVT |
| | | DLHEGHTYVFKVRAVNDAGVGKSSEVSEPVLVEAKPGTKEIFSGVDDEGNI |
| | | YLAFECKEATDASHFAWGKSYEEIEDSDKYKIETEGDRSKLYFKNPDKSDL |
| | | GTYSISVSDTDGVSSSFVMDDEELERLMAISNEIKNPTIPLKSELAYEVLEKG |
| | | EVRFWIQAESLSPDSTFRFVINDKEVENSDRHKINCDRPNGIIEMVMDRFTID |
| | | NEGTYTVQIQDGKAKNQSSLVLIGDAFKAVLAESELQRKEFLRKQGPHFSE |
| | | FLYWEVTEECEVLLACKVANTKKETVFKWYKDGSGIDVDEMPDLQKGEC |
| | | HLSIPRLSRKDEGVYKATLSDDRGHDVSTLELSGKVYDDIILALSRVSGKTA |
| | | SPLKILCTEEGIRLQCFLKYYNEEMKISWSHRESKISSGEKMKIGGGENIAWL |
| | | QITEPTEKDKGNYTFEIFGDKESFKRTLDLSGQAFDDAYAEFQRLKAAAFAE |
| | | KNRGKVIGGLPDVVTIMDGKTLNLTCTVFGNPDPEVVWFKNDKALELNEH |
| | | YVVSLEQGKYASLTIKGVTSEDSGKFSIHVKNKYGGETVDVTVSVYRHGEK |
| | | VPEVKPGQLAKPRPIPPSEGSS |
| 196 | Domestic | MVFKMSILKIHAREIFDSRGNPTVEVDLYTNKGLFRAAVPSGASTGIYEALE |
| | Muscovy Duck | LRDNDKTRYMGKGVSKAVEHVNKTIAPALISKNVNVVEQEKIDKLMLDM |
| | A0A8C3CT67 | DGSENKSKFGANAILGVSLAVCKAGAAEKGVPLYRHIADLAGNPEVILPVP |
| | (Phosphopyruvate | AFNVINGGSHAGNKLAMQEFMILPVGADSFKEAMRIGAEVYHNLKNVIKE |
| | hydratase) | KYGKDATNVGDEGGFAPNILENKEALELLKTAIGKAGYSDKVVIGMDVAA |
| | | SEFYRDGKYDLDFKSPDDPSRYISPDQLADLYKGFVKNYPVVSIEDPFDQD |
| | | DWAAWKKFTGSVGIQVVGDDLTVTNPKRIAKAVEEKSCNCLLLKVNQIGS |
| | | VTESLQACKLAQSNGWGVMVSHRSGETEDTFIADLVVGLCTGQIKTGAPC |
| | | RSERLAKYNQLLRIEEELGSKARFAGRNFRNPRIN |
| 197 | Domestic | MAPKKAKKRIEGANSNVFSMFEQAQIQEFKEAFTIMDQNRDGFIDKADLRD |
| | Muscovy Duck | TFAALGRLNVKNEEIDEMIKEAPGPINFTVFLTMFGEKLKGADPEETILNAF |
| | A0A8C3BXQ0 | KVFDPEGKGLKSAYIKEMLMTQGERFSQEEVDQMFAAFPPDVSGNLDYKN |
| | (Myosin light | LVHVITHGEEKD |
| | chain 2) | |
| 198 | Domestic | MTSPDAEMAAFGEAAPYLRKSEKERIEAQNKPFDAKSSVFVAHPKESFVKG |
| | Muscovy Duck | TIQSKESGKVTVKTEGGETLTVKEDQIFSMNPPKYDKIEDMAMMTHLHEPA |
| | A0A8C3BJT4 | VLYNLKERYAAWMIYTYSGLFCVTVNPYKWLPVYNPEVVLAYRGKKRQE |
| | (Uncharacterized | APPHIFSISDNAYQFMLTDRENQSILITGESGAGKTVNTKRVIQYFATIAASG |
| | protein) | EKKKEEQSGKMQGTLEDQIISANPLLEAFGNAKTVRNDNSSRFGKFIRIHFG |
| | | ATGKLASADIETYLLEKSRVTFQLKAERSYHIFYQIMSNKKPELIDMLLITTN |
| | | PYDYHFVSQGEITVASINDQEELMATDSAIDILGFTADEKVAIYKLTGAVMH |
| | | YGNLKFKQKQREEQAEPDGTEVADKAAYLMGLNSADLLKALCYPRVKVG |
| | | NEYVTKGQTVQQVNNSVGALAKAVYEKMFLWMVVRINQQLDTKQPRQY |
| | | FIGVLDIAGFEIFDFNSLEQLCINFTNEKLQQFFNHHMFVLEQEEYKKEGIEW |
| | | EFIDFGMDLAACIELIEKPMGIFSILEEECMFPKATDTSFKNKLYDQHLGKSS |
| | | NFQKPKPAKGKAEAHFSLVHYAGTVDYNITGWLEKNKDPLNETVIGLYQK |
| | | SSVKTLALLFANYGGAEAEASGGGASGGKKGGKKKGSSFQTVSALFRENL |
| | | NKLMTNLRSTHPHFVRCIIPNETKTPGAMEHELVLHQLRCNGVLEGIRICRK |
| | | GFPSRVLYADFKQRYKVLNASAIPEGQFIDSKKASEKLLGSIDVDHTQYKFG |
| | | HTKVFFKAGLLGLLEEMRDEKLAQLITRTQARCRGFLMRVEYQRMVERRE |
| | | SIFCIQYNVRAFMNVKHWPWMKLFFKIKPLLKSAESEKEMANMKEEFEKT |
| | | KEELAKSEAKRKELEEKMVKLVQEKNDLQLQVQAEADALADAEERCDQLI |
| | | KTKIQLEAKIKEVTERAEDEEEINAELTAKKRKLEDECSELKKDIDDLELTL |
| | | AKVEKEKHATENKVKNLTEEMASLDETIAKLTKEKKALQEAHQQTLDDLQ |
| | | AEEDKVNTLTKAKTKLEQQVDDLEGSLEQEKKLRMDLERAKRKLEGDLKL |
| | | SQDTIMDLENDKQQLDEKLKKKDFEISQIQSKIEDEQALGMQLQKKIKELQ |
| | | ARIEELEEEIEAERTSRAKAEKHRADLSRELEEISERLEEAGGATAAQVEMN |
| | | KKREAEFQKMRRDLEEATLQHEATAATLRKKHADSTAELGEQIDNLQRVK |
| | | QKLEKEKSELKMEIDDLASNMESVSKAKANLEKMCRTLEDQLSEIKTKEEE |
| | | HQRMINDLTAQRARLQTESGEYSRQVEEKDSQISQLSRSKQAFTQQIEELKR |
| | | HLEEEIKAKNALAHALQSARHDCDLLREQYEEEQEAKGELQRALSKANSE |
| | | VAQWRTKYETDAIQRTEELEEAKKKLAQRLQDAEEHVEAVNAKCASLEKT |
| | | KQRLQNEVEDLMIDVERTNAACAALDKKQKNFDKILAEWKQKYEETQAE |
| | | LEASQKESRSLSTELFKMKNAYEESLDHLETLKRENKNLQQEISDLTEQIAE |
| | | GGKAIHELEKVKKQIEQEKSEIQAALEEAEASLEHEEGKILRLQLELNQVKS |
| | | EIDRKIAEKDEEIDQLKRNHLRIVESMQSTLDAEIRSRNEALRLKKKMEGDL |
| | | NEMEIQLSHANRQAAEAQKNLRNTQAVLKDTQIHLDDALRTQEDLKEQVA |
| | | MVERRANLLQAEIEELRAALEQTERSRKVAEQELMDASERVQLLHTQNTSL |
| | | INTKKKLETDIVQIQGEMEETIQEARNAEEKAKKAITDASMMAEELKKEQD |
| | | TSAHLERMKKNLDQTVKDLQHRLDEAEQLALKGGKKQIQKLEARVRELEG |
| | | DVDAEQKRSAEAVKGVRKYERRVKELTYQSEEDRKNILRLQDLVDKLQM |
| | | KVKSYKRQAEEAEELSNVNLSKFRKIQHELEEAEERADIAESQVNKLRAKS |
| | | REFHGKKIEEEE |
| 199 | Domestic | MTSPDAEMAAFGEAAPYLRKSEKERIEAQNKPFDAKSSVFVAHPKESFVKG |
| | Muscovy Duck | TIQSKESGKVTVKTEGGETLTVKEDQIFSMNPPKYDKIEDMAMMTHLHEPA |
| | A0A8C3BJT4 | VLYNLKERYAAWMIYTYSGLFCVTVNPYKWLPVYNPEVVLAYRGKKRQE |
| | (Uncharacterized | APPHIFSISDNAYQFMLTDRENQSILITGESGAGKTVNTKRVIQYFATIAASG |
| | protein) | EKKKEEQSGKMQGTLEDQIISANPLLEAFGNAKTVRNDNSSRFGKFIRIHFG |
| | | ATGKLASADIETYLLEKSRVTFQLKAERSYHIFYQIMSNKKPELIDMLLITTN |
| | | PYDYHFVSQGEITVASINDQEELMATDSAIDILGFTADEKVAIYKLTGAVMH |
| | | YGNLKFKQKQREEQAEPDGTEVADKAAYLMGLNSADLLKALCYPRVKVG |
| | | NEYVTKGQTVQQVNNSVGALAKAVYEKMFLWMVVRINQQLDTKQPRQY |
| | | FIGVLDIAGFEIFDFNSLEQLCINFTNEKLQQFFNHHMFVLEQEEYKKEGIEW |
| | | EFIDFGMDLAACIELIEKPMGIFSILEEECMFPKATDTSFKNKLYDQHLGKSS |
| | | NFQKPKPAKGKAEAHFSLVHYAGTVDYNITGWLEKNKDPLNETVIGLYQK |
| | | SSVKTLALLFANYGGAEAEASGGGASGGKKGGKKKGSSFQTVSALFRENL |
| | | NKLMTNLRSTHPHFVRCIIPNETKTPGAMEHELVLHQLRCNGVLEGIRICRK |
| | | GFPSRVLYADFKQRYKVLNASAIPEGQFIDSKKASEKLLGSIDVDHTQYKFG |
| | | HTKVFFKAGLLGLLEEMRDEKLAQLITRTQARCRGFLMRVEYQRMVERRE |
| | | SIFCIQYNVRAFMNVKHWPWMKLFFKIKPLLKSAESEKEMANMKEEFEKT |
| | | KEELAKSEAKRKELEEKMVKLVQEKNDLQLQVQAEADALADAEERCDQLI |
| | | KTKIQLEAKIKEVTERAEDEEEINAELTAKKRKLEDECSELKKDIDDLELTL |
| | | AKVEKEKHATENKVKNLTEEMASLDETIAKLTKEKKALQEAHQQTLDDLQ |
| | | AEEDKVNTLTKAKTKLEQQVDDLEGSLEQEKKLRMDLERAKRKLEGDLKL |
| | | SQDTIMDLENDKQQLDEKLKKKDFEISQIQSKIEDEQALGMQLQKKIKELQ |
| | | ARIEELEEEIEAERTSRAKAEKHRADLSRELEEISERLEEAGGATAAQVEMN |
| | | KKREAEFQKMRRDLEEATLQHEATAATLRKKHADSTAELGEQIDNLQRVK |
| | | QKLEKEKSELKMEIDDLASNMESVSKAKANLEKMCRTLEDQLSEIKTKEEE |
| | | HQRMINDLTAQRARLQTESGEYSRQVEEKDSQISQLSRSKQAFTQQIEELKR |
| | | HLEEEIKAKNALAHALQSARHDCDLLREQYEEEQEAKGELQRALSKANSE |
| | | VAQWRTKYETDAIQRTEELEEAKKKLAQRLQDAEEHVEAVNAKCASLEKT |
| | | KQRLQNEVEDLMIDVERTNAACAALDKKQKNFDKILAEWKQKYEETQAE |
| | | LEASQKESRSLSTELFKMKNAYEESLDHLETLKRENKNLQQEISDLTEQIAE |
| | | GGKAIHELEKVKKQIEQEKSEIQAALEEAEASLEHEEGKILRLQLELNQVKS |
| | | EIDRKIAEKDEEIDQLKRNHLRIVESMQSTLDAEIRSRNEALRLKKKMEGDL |
| | | NEMEIQLSHANRQAAEAQKNLRNTQAVLKDTQIHLDDALRTQEDLKEQVA |
| | | MVERRANLLQAEIEELRAALEQTERSRKVAEQELMDASERVQLLHTQNTSL |
| | | INTKKKLETDIVQIQGEMEETIQEARNAEEKAKKAITDASMMAEELKKEQD |
| | | TSAHLERMKKNLDQTVKDLQHRLDEAEQLALKGGKKQIQKLEARVRELEG |
| | | DVDAEQKRSAEAVKGVRKYERRVKELTYQSEEDRKNILRLQDLVDKLQM |
| | | KVKSYKRQAEEAEELSNVNLSKFRKIQHELEEAEERADIAESQVNKLRAKS |
| | | REFHGKKIEEEE |
| 200 | Domestic | MKWVTLISFIFLFSSATSRNLQRVARDADHKSEIAHRYNDLKEETFKAVAMI |
| | Muscovy Duck | TFAQYLQRCSYDGLSKLVKDVVDLAQKCVANEDAPECSKSLPSIFLDEICQ |
| | A0A8C3CNB8 | VEKLRDSYGAMADCCAKSDPERNECFLSFKIPQPDFVQPYQRPASDVICKE |
| | (Albumin) | YEDNRVSFLGHFIYSVARRNPFLYAPTILGVAADYEHALKSCCKESDVGAC |
| | | LDGKETAIREKVKKISVKQQYSCGILQKFGDRIFQADKLAFLSQKYPKASFT |
| | | EISKLIHDVKDVYKECCEGDMVECMDDRAELVNYICSKHESFSSKIKDCCE |
| | | KPIVERSQCIMEAEFDEKPADLPSLVEKYIQDKEVCKSFEAGHDAFMSEFVY |
| | | EYSRRHPEFSTQLILRIAKGYETLLEKCCKTDNPAECYANAQDQLNEHIKET |
| | | QDVVKTNCDLLTTHGEADFLKALLIRYTKKMPQVSTETLLEIGKKMTGVG |
| | | TKCCKLPEDRRLPCSEGYLSMVIQDMCRRQETSPINDNVSHCCSDSYAYRR |
| | | PCFSAMGVDTKYVPPPFNPEMFNFDEKLCSAPAEERAAGQMKLLVNLIKRK |
| | | PQMTEEQIKTIADGFTAMVDKCCKQSDIETCFGEEGANLIVQSRATLGIGA |

An animal protein can comprise a salmon protein (Table 12). A salmon protein can comprise a salmon muscle protein. A salmon protein can comprise a salmon blood protein. A salmon protein can comprise a salmon albumin protein. A salmon protein can comprise a sequence that has at least or about 70%, at least or about 75%, at least or about 80%, at least or about 90%, at least or about 91%, at least or about 92%, at least or about 93%, at least or about 94%, at least or about 95%, at least or about 96%, at least or about 97% at least or about 98% at least or about 99% or about 100% sequence identity to any one of SEQ ID NO: 201-216.

A food product may comprise a fragment or a truncation of a salmon protein. The food product may include a fragment or truncation of any one of SEQ ID Nos: 201-216. A sequence of the fragment or truncation may be identical or substantially identical to a portion of any one of SEQ ID Nos: 201-216. A sequence of the fragment or truncation may be at least about 70%, 80%, 90%, 95%, 98%, or more identical to a portion of a sequence of any one of SEQ ID Nos: 201-216. A length of the fragment or truncation may be at least about 10%, 15%, 20%, 30%, 40%, 50%, 60%, 70%, 80%, 90%, or more of a length of any one of SEQ ID Nos: 201-216. A length of the fragment or truncation may be less than or equal to about 90%, 80%, 70%, 60%, 50%, 40%, 30%, 20%, 15%, 10%, or less than a length of any one of SEQ ID Nos: 201-216.

TABLE 12
Salmon Proteins

| SEQ | | |
|---|---|---|
| ID NO | Description | Amino Acid Sequence |
| 201 | Salmon B5DGP0 | MPFGNTHNNFKLNFKVEEEYPDLTKHNNHMAKVLTKDMYAKLRDKQTPSG |
| | (Creatine kinase) | FTLDDVIQTGVDNPGHPFIMTVGCVAGDEESYEIFKDLLDPIISDRHSGYKPT |
| | | DKHKTDLNFENLKGGDDLDPNYVLSSRVRTGRSIKGYTLPPHNSRGERRAV |
| | | EKLSVEALNTLDGEFKGKYYPLNKMTDAEQEQLIADHFLFDKPVSPLLLGA |
| | | GMARDWPDARGIWHNDAKSFLVWVNEEDHLRVISMEKGGNMKEVFRRFC |
| | | VGLKRIEETFKKHNHGFMWNEHLGYVLTCPSNLGTGLRGGVHVKLPKLSTH |
| | | PKFEEILTRLRLQKRGTGGVDTASVGGVFDISNADRLGSSEVDQVQMVVDG |
| | | VKLMVEMEKKLEKGEAIDGMIPAQK |
| 202 | Salmon | MTAIETQVQYGSYMTSEQVYMTQEDDWDRDLLLDPAWEKQQRKTFTAWC |
| | A0A1S3RGX6 | NSHLRKAGTQIENIEEDFRNGLKLMLLLEVISGERLPKPDKGKMRFHKIANV |
| | (Alpha-actinin-3) | NKALDFICSKGVKLVSIGAEEIVDGNVKMTLGMIWTIILRFAIQDISVEETSAK |
| | | EGLLLWCQRKTAPYRNVNVQNFHISWKDGLALCALIHRHRPDLIDYSKLRK |
| | | DDPMGNLNTAFEVAEKYLDIPKMLDAEDIVNTPKPDEKAIMTYVSCFYHAF |
| | | AGAEQAETAANRICKVLAVNQENEKLMEEYEKLASELLEWIRRTIPWLENR |
| | | VAEQTMRAMQQKLEDFRDYRRVHKPPKVQEKCQLEINFNTLQTKLRLSNRP |
| | | AFMPSEGKMVSDIANAWKGLEQVEKGYEEWLLTEIRRLERLDHLAEKFKQK |
| | | SSLHESWTSGKVELLSMKDYESASLMEIRALMRKHEAFESDLAAHQDRVEQ |
| | | IAAIAQELNELDYHDAVTINARCQGICDQWDNLGTLTQKRRDSLERVEKLW |
| | | ETIDQLYLEFAKRAAPFNNWMDGAMEDLQDMFIVHSIEEIQSLITAHDQFKA |
| | | TLPEADKERVATMGIQNEIVKIAQTYGIKLSGVNPYTNLSPQDITDKWDAVK |
| | | HLVPLRDQMLQEEVARQQSNERLRRQFAAQANIIGPWIQTKMEEISHVSVDI |
| | | AGSLEEQMSNLKQYEQNIINYKCNIDKLEGDHQLSQESLIFDNKHTNYTMEH |
| | | VRVGWEQLLTTIARTINEVENQILTRDAKGISQEQLNEFRASFNHFDRKRNG |
| | | MMDPDDFRACLISMGYDLGEVEFARIMTLVDSNNTGVVTFQAFIDFMTRET |
| | | AETDTADQVMASFKILASDKTYITVEELRRELPPEQADYCISRMTSYIGSGAP |
| | | PGALDYISFSSALYGESDL |
| 203 | Salmon | MPKPLTDQDKRKQISVRGLAGVENVSDLKTNFNRHLHFTLVKDRNVATKR |
| | C0H8W5 | DYYFALANTVRDHLVGRWIRTQQHYYEKDPKRVYYLSLEFYMGRTLQNTM |
| | (Alpha-1,4 | VNLALENACDEATYQLGLDMEELQDIEEDAGLGNGGLGRLAACFLDSMAS |
| | glucan | LGLAAYGYGIRYEFGIFNQKIVNGWQVEEADDWLRYGNPWEKARPEYMRP |
| | phosphorylase) | VHFYGRVEHTPEGVKWVDTQVVLALPYDTPVPGYRNNVVNTMRLWSAKA |
| | | PCDFNLKDFNVGGYIQAVLDRNLAENISRVLYPNDNFFEGKELRLKQEYFVV |
| | | AATLQDIVRRFKSSKFGSTEVVRVDLSTLPDKVAIQLNDTHPAMAIPELMRIL |
| | | LDTEHQTWEKAWDICTRTCAYTNHTVLPEALERWPTDLLQNLLPRHLEIIYE |
| | | INRRHLERIAKLYPGDHDRLRRMSLVEEGDQKKINMAHLCIVGSHAVNGVA |
| | | RIHSDILKATLFKDFYEVDPHKFQNKTNGITPRRWLVMCNPGLAEVIAERIGE |
| | | DYIRDLDQLKKLLTFVDDDSLIHDIAKVKQENKLKFSAYLEEHYKVKINPNS |
| | | MFDVQVKRIHEYKRQLLNCLHIITLYNRIKKEPNKNWTPRTIMIGGKAAPGY |
| | | HTAKLIIRLITAIGEIVNHDTVVGDRLKVIFLENYKVTLAEKIIPASDLSEQIST |
| | | AGTEASGTGNMKFMLNGALTIGTMDGANVEMAEEAGEENLFIFGMRVDDV |
| | | DAMDKSGYDAMDYYNRIPELKQAMDQIAGGFFSPDQHDLFKDIVNMLLHH |
| | | DRFKVFADYEAYIKCQEKVSALYKNPKEWTKMVIHNIAGCGKFSSDRTISQY |
| | | AREIWGMEPSLERIPAPDDPRQ |
| 204 | Salmon C0H911 | MPHSFPFLSTEQKKELSDIAQKIVAPGKGILAADESTGSVAKRFQSINAENTE |
| | (Fructose- | ENRRLYRQLLFTADRVEPCIGGVIFFHETLYQKTDDGKLFPQLIKERDMVVGI |
| | bisphosphate | KVDKGVVPLAGTNGETTTQGLDGLYERCAQYKKDGADFAKWRCVLKITDT |
| | aldolase) | TPSELAIKENANVLARYASICQMHGIVPIVEPEILPDGDHDLKRCQYVTEKVL |
| | | AAVYKALSDHHVYLEGTLLKPNMVTPGHSCSQKYSNQEIAMATVTALRRT |
| | | VPPAVPGITFLSGGQSEEEASINLNAMNQCPLHRPWAITFSYGRALQASALK |
| | | AWGGKPENGKACQEEFIKRALNNSKACQGKYVSSGDKGAAGGESLYVANH |
| | | AY |
| 205 | Salmon B5DH12 | MAPKKDAKAPAKKAEPAKAAAPAPAPEPDVVAAPPPLDLSTVKVEFTPDQQ |
| | (Myosin light | EDYKEAFGLFDRVGDAKVAYNQVADIMRALGQNPTNKEVRKVLGNPSDED |
| | chain 1-1) | MAAKRLEFEAFLPMLQHIVNDPNKGTEDDYVEGLRVFDKEGNGTVMGAEL |
| | | RIVLGTLGEKMSEAEIDALMQGQEDENGSINFEAFVKHIMSI |
| 206 | Salmon | MVKVGVNGFGRIGRLVTRAAFHSKKGVEIVAINDPFIDLDYMVYMFKYDST |
| | B5DGR3 | HGRFHGEVKAEGGKLVIDGHKITVFHERDPANIKWGDAGATYVVESTGVFT |
| | (Glyceraldehyde- | TIEKASTHLKGGAKRVVISAPSADAPMFVMGVNHEKYDNSLKVVSNASCTT |
| | 3-phosphate | NCLAPLAKVIHDNYHIIEGLMSTVHAVTATQKTVDGPSGKLWRDGRGASQN |
| | dehydrogenase) | IIPASTGAAKAVGKVIPELNGKITGMAFRVPTPNVSVVDLTVRLEKPASYDAI |
| | | KKVVKAAADGPMKGILGYTEQQVVSSDFNGDTHSSIFDAGAGIALNDHFVK |
| | | LVTWYDNEFGYSNRVIDLMAHMATKE |
| 207 | Salmon Q91472 | MDAIKKKMQMLKLDKENALDRAEGAEGDKKAAEDKSKQLEDDLVALQKK |
| | (Fast myotomal | LKGTEDELDKYSESLKDAQEKLEVAEKTATDAEADVASLNRRIQLVEEELD |
| | muscle | RAQERLATALTKLEEAEKAADESERGMKVIENRASKDEEKMELQDIQLKEA |
| | tropomyosin) | KHIAEEADRKYEEVARKLVIIESDLERTEERAELSEGKCSELEEELKTVTNNL |
| | | KSLEAQAEKYSQKEDKYEEEIKVLTDKLKEAETRAEFAERSVAKLEKTIDDL |
| | | EDELYAQKLKYKAISEELDNALNDMTSI |
| 208 | Salmon | MPEAKKPAAKTDKAAAAPADKEARSAIEEGSECRDELPDDETVPGGQSELT |
| | A0A1S3RC27 | GLFVERPESTTVIKGKNVTFVAKVDSSDLLRKPNMKWLKGKWLDLGSKAG |
| | (Myosin-binding | KHVQFKEAYDRNSKVYTYEMSIIKVVEGDAGGYRCEVTSKDKCDSCTFEVT |
| | protein C) | VEAVQEEQPANILDAFKRSGDAGEDAGDLDFSALLKKREKKARDEPKEDVD |
| | | VWEILKDAKPCDYEKIAFDYGITDLRGLLKRLKKMKKVEPKKSDAFLKKLE |
| | | QCYSVDKGKRTQMHVELHDPNVQVKWLKNGVEIKPSAKYVFECVGNKRTL |
| | | TINKCNLSDDAAYECVVGEEKSFTEVFVKEPPVTITKLLDDVHVVVGEKVEF |
| | | ECEVSEEGANVKWMKDGVELTKDGKIRIKKDGKKHTLVINEATIEDIGMYY |
| | | VYTNGGESKGELEVEAKELEVLQSIADLSVKACEQAMFKCEVSDEKVVGK |
| | | WFKDGVEVKPGNRIKMSHIGRIHKLTIDDVKPQDEGNYTFVPEGYALSLSAK |
| | | LNFIEIKIEYVPRQDPPKIHLDTSSTGSKNTIVVVAGNKLRLDVEITGEPVPTV |
| | | CWMKGDTVISEAEGRVRVETRTTLSSFVIEGAERPDEGQYSIIVTNPAGEDRA |
| | | ELTVKIVDVPNPPENVRCMGVGEDTATITWDPPKFDGGAPIKGYLMERKKQ |
| | | GSSRWTKLNFEVFESTTYEAKKMIEGVFYEMRVFAVNGIGISQPSGNSKPFM |
| | | PIAPTSEPTRLTVEDVTDSTCALKWRPPERVGAGGVDGYIIEWCKEGEDNWV |
| | | AANKEPVDKNTYRVKGLPTGEKLLFRVVAMNIAGRSPPCTMKQSVTIREIM |
| | | EYPKIRLPRQLRTKFIRKVGEKINLVIPFQGKPRPVVNWLKDGEPLENKSVGI |
| | | RTSDFDTILFIRSAERDHSGTYTLSVQIDNMQDKADIHIQIVDKPGPPINVMVT |
| | | DVWGFNAALEWKPPKDTGNTDITGYTIQKADKKTQGWFTVYEHNRRPSCT |
| | | ASDLVMGNEYSFRVFSENICGLSDEVAFSKNTAIIGKTDLEYNKPAFKERDM |
| | | SSSPKFTAPLVDRVVVAGYSTAISCAVRAYPKAKIVWMKNKMIIGEDPKFLM |
| | | QNNQGVLTLNIRKPGQFDGGKYSCKAINDLGEDEVECRLEVRVLKEKKEGD |
| | | EEKK |
| 209 | Salmon | MATKVMHFNQKKHVSHVSHHSSHTTKHVVKEQSSRKSSSKSVNQVQHTHA |
| | A0A1S3L6A0 | TEAMAEPAYTVPAFRQRSADEIEEYQRVSSHVGKGLASIQKELHRMRVVTK |
| | (Myomesin-2 | TQVENIAIKREVQEMMHKKMTLSTETDKAPDFMVALRPHTVWEKTPVKLF |
| | isoform X1) | CTVDGHPRPIVKWYKGGKPVDPLSAPGKYKIESKYGVHSLIISRCMVSDTAE |
| | | YSAVATNSHGSTTSKASVIVKRPAGGAYGSCQLFGQVPHLTVIHHSKLEITM |
| | | LDRFGVSFGTEGGSISLVCTMVVVPDLPNVPPLAQWYRDDKLLKAGKLAEI |
| | | KVGGGAATLTLPHLAKDDEGLYTLRMWTKDGTTEHSAYLFVKDAAPSVAG |
| | | APGAPISVKAFDINSDYVLVAWKPPNTTNEAAITGYFVDKRESGSATWSQCN |
| | | DAPVQICKYPVHGLNVGHAYHFRVRAVNSAGISRPSRESDKVTALDPAERE |
| | | RLQVIKLDGKHEVVIKDDDLEGYVTLPGEPTDMHASEIFKSYIVLSWKPPSPR |
| | | GRAPLWYIIEKSVSGTGAWQRINTAVKLQSPRYPVFDLEAGKKYQFRVYSE |
| | | NLYGASEASKPTDPIEKVDEHGVPSAPGQVVATRNTKSSVFVQWDPPKHPN |
| | | HLMGYYIDASLVGSKTWAPCNHKPYKNTRFVVHGLTPGETYVFRVQAVNV |
| | | YGLSDESQESTPIAVEPALTTPSAPHGITMLSCDGASMIIAWNSPKHRGGSKI |
| | | NAYYVDKRDADSLAWKEVNSAPTNTRTYTVDGLTEGTFYEFKVQAGNLAG |
| | | VGVPSAPSTPLKCEAWTSAVPGPAYDLAFREVRGDSLVILWKAPVYTGASA |
| | | VTGYIVEMAKKGHHYHPLNEEAIGHCYLQVTGLEAGAEYTFKVKAVNAEG |
| | | VGKASQTSEPVFAKALPGTQEIQCGVDEDTGDIFLSFEACEISETSNFAWSKN |
| | | YKEISDCTRVSIETKGKHSKLTFVNPDVSDLGAFSVHVTDTDGVSASHTVTE |
| | | DALNTMLELSYNIRHPIVPLKHQLNYEVLEKGHVRFWLQCLKMSPAVNYRF |
| | | IVNDKEVTSSDSHKISHDVNTGVIQMTVDHFSKASEATYTVQIHDGRAKNQS |
| | | SLVLVGDVFKAALKEAEFQRSEHIRKQGPHFAEYLSYHVDDDCTVLLVCKV |
| | | ANVKKETVFHWFKDEEEIVPETPPNVMSGSVALPISLFSRKDQGHYKAKLSD |
| | | DRGKDTSVFDISGQVFDDIINAIAHIAGSSASELVLQCTPEGIRLQCYMKYYT |
| | | EEIRTAWLHKDSKISSSEKMRIAGTAEMAWMQIREPSEKEKGHYSIQIMDAT |
| | | KSHTRTFDLSGQAYTDAYEEYLRLKAAAFAEKNRGRVVGGLPDVVTIMEQ |
| | | KTLSLTCTVWGDPSPEVTWFKNENEVVSTEHAKITLEANKFASLTITAVTSE |
| | | DSGKYSINVRNKYGGEFVEITVSVYKQGESPPEPKMGGRGATPAPLASKTPA |
| | | KTPTPAQTPTPSLKSPTPSLKSPTPSLRSPAKSPTPTPKSPTPPRRVKSPSPARSL |
| | | KSPTPPRK |
| 210 | Salmon ENOB | MSITKIHAREILDSRGNPTVEVDLYTAKGRFRAAVPSGASTGVHEALELRDG |
| | (Beta-enolase) | DKSRYLGKGTVKAVDHVNKDIAAKLIEKKFSVVDQEKIDHFMLELDGTENK |
| | | SKFGANAILGVSLAVCKAGAAEKGVPLYRHIADLAGHKDVILPCPAFNVING |
| | | GSHAGNKLAMQEFMILPIGASNFHEAMRIGAEVYHNLKNVIKAKYGKDATN |
| | | VGDEGGFAPNILENNEALELLKTAIEKAGYPDKIIIGMDVAASEFYKAGKYD |
| | | LDFKSPDDPARYITGDQLGDLYKSFIKGYPVQSIEDPFDQDDWAAWTKFTAA |
| | | VDIQVVGDDLTVTNPKRIQQAVEKKACNCLLLKVNQIGSVTESIKACKLAQS |
| | | NGWGVMVSHRSGETEDTFIADLVVGLCTGQIKTGAPCRSERLAKYNQLMRI |
| | | EEELGAKAKFAGKDYRHPKIN |
| 211 | Salmon Q7ZZN0 | MAPKKAKRRGAAAEGGSSNVFSMFEQSQIQEYKEAFTIIDQNRDGIISKDDL |
| | (Myosin | RDVLASMGQLNVKNEELEAMVKEASGPINFTVFLTMFGEKLKGADPEDVIV |
| | regulatory light | SAFKVLDPEATGFIKKEFLQELLTTQCDRFSAEEMKNLWAAFPPDVAGNVD |
| | chain 2) | YKQICYVITHGEEKEE |
| 212 | Salmon | MTTLGLTEGHPAKYQHLTNLVPAISYELGQKKERVIMSTDAEMAAFGPAAI |
| | A0A1S3LA68 | YLRKPERERIASQAAPFDAKTAFFVVEPKEMYLKGVLQSKEGGKATVKTLC |
| | (Myosin heavy | NKVLTVKEDDIHPMNPPKYDKIEDMAMMTHLNEATVLYNLKERYAAWMI |
| | chain) | YTYSGLFCVTVNPYKWLPVYDSVVVNAYRGKKRIEAPPHIFSISDNAYQFML |
| | | TDHENQSILITGESGAGKTVNTKRVIQYFATIAVAGGKKEQTAAATSGKIKG |
| | | SLEDQIIAANPLLEAYGNAKTVRNDNSSRFGKFIRIHFGTTGKLASADIETYLL |
| | | EKSRVTFQLSAERSYHIFYQLMTGHQPQLLEALLITTNPYDYPIISHGEIAVKS |
| | | IDDTEEFIATDTAIDILGFTAEEKIGIYKLTGSVMHHGAMKFKQKQREEQAEP |
| | | DGTEEADKISYLMGLNSADLLKALCYPRVKVGNEMVTKGQTVPQVNNSTM |
| | | ALCKSVYEKMFLWMVVRINEMLDTKQSRQFFIGVLDIAGFEIFDYNSLEQLC |
| | | INFTNEKLQQFFNHHMFVLEQEEYKKEGIEWAFIDFGMDLAACIELIEKPMGI |
| | | FSILEEECMFPKASDTTFKSKLYDQHIGKTKAFEKPKPGKGKAEAHFALVHY |
| | | AGTVDYNVTGWLDKNKDPLNDSVVQLYQKSSVKLLAMLYVGAAASQADD |
| | | AARGGGGKKKKGGSFQTVSALFRENLGKLMTNLRSTHPHFVRCLIPNESKTP |
| | | GLMENFLVIHQLRCNGVLEGIRICRKGFPSRIQYGDFKQRYRVLNASVIPDGQ |
| | | FIDNKKASEKLLGSIDVDKSQYKFGHTKVFFKAGLLGTLEEMRDEKLATLVT |
| | | MTQALCRGYVMRKEFVKMMARREAIYSIQYNIRSFMNVKHWPWMKLYFKI |
| | | KPLLKSAETEKEMAAMKENFEKMKEDLAKALAKIKGLEEKIVSLLQENNDL |
| | | QLQIAAEENTLCDAEERCEGLIKSKIQMEAKLKETFERLEDEEELNAELTAKK |
| | | RKLEDECSELKKDIDDLELTLAKVEKEKHATENKVKNLTEEMSSQDESLIKL |
| | | TKEKKALQEVHQQVLDDLQAEEDKVNNLTKAKTKLEQQVDDLEGSLEQEK |
| | | KVRMDLERAKRKLEGDLKLALESLMDLENDKQQSDEKIKKKDFETSQLLSK |
| | | IEDEQALGAQLQKKIKELQARIEELEEEIEAERAARAKVEKQRSDLSRELEEIS |
| | | ERLEEAGGATSVQIEMSKKREAEFQKLRRDLEESTLQHEATAAALRRKQAD |
| | | TVAELGEQIDNLQRVKQKLEKEKSEYKMEIDDLSSNMEAIAKAKGNLEKMC |
| | | RSLEDQLSELKTKNDEHVRQLNDINVQRARLLTENGELGRQMEEKESLVSQ |
| | | LTRAKQTFTQQIEDLKRQIEEEVKAKNALAHGLQSARHDCDLLREQYEEEQE |
| | | AKAELQRALSKANSEVAQWRSRYETDAIQRTEELEDAKKKLTQRLQDAQE |
| | | AVEATNAKCASLEKTKQRLLGEVEDLMIDVERANASAAQLDKKQRNFDKV |
| | | LSEWKQKYEEGQAELEGSLKEARSLSTELFKMKNSYEEALDHLETLKRENK |
| | | NLQHEISDLTELVGESGKSIHELDKAKKTVENEKAEIQAALEEAEGTLEHEES |
| | | KILRIQLELNQIKGEVDRKLAEKDEETEQIKRNSQRMMDSMQSTLDSEIRSRN |
| | | DAMRVKKKMEGDLNEMEIQLSHANRQAAESQKQLRNVQGALKDAQLQLD |
| | | DAIRVADDMKEQVAMVERRNNLMMAEIEELRAALEQTERGRKVAEQELVD |
| | | ASERVGLLHSQNTSLINTKKKLEVDLTQVQGEMEDTVQEARNAEEKAKKAI |
| | | TDAAMMAEELKKEQDTSSHLERMKKNLEVTVKDLQHRLDEAENLAMKGG |
| | | KKQLQKLEARVRELESEVDAEQKRGVEAIKGVRKYERRVKELTYQTEEDNK |
| | | NVSRLQDLVDRLQLKMKAYKRAAEDAEEQSNIHLSRYRKVQHELEEAQER |
| | | ADISESQVNKLRAKSREIGSKGNVAEE |
| 213 | Salmon | MDENEEFEEEFEEEEIIEEEYFEETVTSTTSHQTTSRAAAVTQQPGVPVRRVV |
| | A0A1S3NZV0 | RKKVKVDTSKFMTPYLAHSQKMLDLFSYNKYRRAYDKAKGKPYAITADTP |
| | (Nebulin isoform | EMMRIKKAQEQLSEVKYRMEGNKVRCTSLYDGEAREIAHVKHVSELISKVL |
| | X4) | YRAKWDETKDRYLLPPDAPELVLAVKNAANYSKKLYTEQWDEDKTMCYP |
| | | YSDSPELRRVAKAQEVLSDILYKKGHAERTAKYTSLAVPNDMELAKKNFEH |
| | | RSDLKYHEDYNKNVKGHWCETPYIEVAVARVAMDNLSNKMYTAAYEDIK |
| | | DQINFMQTETPEYKQHKKAGHAASSIKYKKDYENSKSVADYNVLPATDNPL |
| | | LRQLRYAGTILSDKVYKASYEKSRGSSINYCDTPKFKMDSVVQQFSDAHYK |
| | | DKYENEVKGHYIGSYEDIYMQHCKKMEEIKSEQNYKADYEDIKTRCFYPQT |
| | | MSPEYEAHKKVAQCKDKVYRQHPDTVKFTQVTDSPVQLQAAINAKQLSDL |
| | | NYHAAYELDKFKCALPPDYPFFIQSRANAYNLSDSCYRYDWEKVKAKQFEV |
| | | KGDAISILAARAHTNIASDVKYKKEFEKNKGKMVGALSIHDDPKILHSMHV |
| | | AKIQSDREYKKNYEKTKTKYITTLDMMSNTLAKKSQGIASMTGYRTIHNRY |
| | | LLPHDAMSLDLAKKANIIQSDNEYHSDYNNSTKGTGWVAFGSMEVEKAKK |
| | | AGDILNERKYRQHPDTVPFTSIADHPVMMQAQVNQIMRSDLHYKKGLEEIH |
| | | QKYSLPHNDPQFLQAKCNAYNISKNYYKQSWEETIAKGYDMRPDAISVIHA |
| | | KHGRHIASDIQYKKDYVKGKGKQIGCRNLQDDPLLVHYMEVARLQSDKNY |
| | | KKDYHKSKLKYHSPVDMMSVVHAKHASTVQTFAGYRKIQHHYSLLPGAM |
| | | NLELARTMNTNASDVNYKADWENSVKGTGWIPIGSIPVELAKQAGAVLDEH |
| | | KYRQHPENFKFSSKMDGMNMELCKHNQKIMNVQEYKASGEKFLHTYNLPA |
| | | DAPEFMQAKCNAVNISKNYYTYAHRQDILKGHQVKADAIPILAARACTNIAS |
| | | NYKYKLAYEKGRGKHVGFRNVKDDPLLVHYMEVAKMQSEQNYKKDYHK |
| | | SKLKYHSPVDMMSLVHAKHASAAQTFAGYRKSLHNYTLLPEAMPVQLART |
| | | MMEIQSNNAYKSDYNNMCRGSGWVPIGSMQVELAKTAMAARDEQGYRVH |
| | | PSTLKFTSLTGQMNMALAMTNTKLLDDASYRASGEKFKHTYNLPADSPQFL |
| | | QAKCNAQTMSESHYKHKWVEDIAKGYDIRPDAISVIHAKHGRHIASDIQYK |
| | | KDYVKGKGKQIGCRNLQDDPLLVHYMEVAKMQSDKNYKKDYHKSKLKYH |
| | | SPVDMMSLEHAKHASTVQTFAGYRKIQHHYTLTADSMNLALARTMNASSS |
| | | DYEYKSDWNNSVRGTGWIPIGSLPVELAKVGGDIQSEQKYRTHPSKFQFKKL |
| | | MDSMDIVLATDNNKIMNKQEYTKQWHNDKLTVHVMPDAPEIVLAKANAIN |
| | | MSQKTYKAGLAKMYKNGYDLKVDAIAIQAAKQGTKIASDYKYKLSHRMN |
| | | RGKQIGCRNLQDDPLLVHYMEVARLQSDKNYKKDYHKSKLKYHSPVDMM |
| | | SVVHAKHASTVQTFAGYRKIQHHYTVLADAMNVELAKQMQTIASDNLYKS |
| | | DYETSVKGTGWIPIGSLDVEKAKTAGLIGSEKLYRQHPSSFKFTKDMHSMDV |
| | | VLANANNAIMNKQTYVDAWNKDKTSVHIMPDSMEVVLARANKINCSATLY |
| | | KLANEQDKKKGYDLRNDAIAILAAKQGTSIASDFKYRAGYRKQVGHHIGAR |
| | | CVEEDPLLMLAINSAKIASDALYKKDFHQSKTKFHLPLDMIAFELAKKNQIQ |
| | | VNHAKYVTRLHNWTCLPDSTDVVHARHVYDIASDAVYKADLKCLQGIGW |
| | | VPIGSLDVEKAKKAGEVLSEKLYRQHPSNFKFTSSVQDIGMVLAKQNNDNA |
| | | NKQAYTEAWETDKTKNHIMPDAMDIVLANANKSNYSWNQYRKAMVEDK |
| | | KKGYDMRNDAISIIAAKHGREIISDHKYKSSYRKQVGHHIGARCVEDDPLLV |
| | | LALNSAKIASDALYKKDFHQSKTKFHLPLDMIAFELCKKNQIQVNDFNYRTH |
| | | LHNWTCLPDSTDVVHARHVYDIASEAVYRADMKWLRGIGWVPTGSLDVEK |
| | | AKKAGEILSEKKYRQHPSTLDFKSPLDGMDIVLAKHNGQIMNKRQYVEQW |
| | | DADKIKIHVGNELPELVLAKANAITMSNKLYKQGFDETIKKGYYLPVDAISV |
| | | KAAKHGREIISDYKYKAGYSKQVGHHIGARCVEEDPLLMLAINSAKIASDAL |
| | | YKKDFHQSKTKFHLPLDMIAFELCKKNQIQVNDFNYRTHLHNWTCLPDSTD |
| | | VVHARRVYDLHSDAVYRADMEFIRGTGWVPIGSLDVLKAKKAAEILSDRLY |
| | | KQKPDSLKYTTDMHSMPMVLAKANAEIINKGNYVAQWEADKIKIHVGNEL |
| | | PELVLAKANAINNSYKLYKAELEELYKKGYNLKSDCIAIKAAKHGREIISDY |
| | | KYKAGYSEQVGHHIGARCVEEDPLIMLALNSAKIASDALYKKDFHQSKTKF |
| | | HLPVDMLTIELAKKCQMQVNDFNYRTRLHNWTCLPDSSDVVQARKVYALH |
| | | SDAVYRADMEWLKGTGWVPIGSIDVEKAKKAGEILSERKYREHPSNFKFTA |
| | | TTDDMPYALATANAQIMDKRQYVQQWEADKIQIHVGNELPELVLAKANAI |
| | | TMSNKLYREGFNEMIGQGYHLDKDAIAVQHAKKSREIISDYKYKAGFRNQC |
| | | GHHIGAHCVEDDPLLVLAANAARISSDINYKKDFHLTKTKFSIPLDMMSFDL |
| | | AKKSQFQIQEFTYRTKLHNWTCMPDNTDVVRARKVYSLHSDAVYRADLEW |
| | | LRGCGWLPQGSVDVIKAKNAQTILNERLYRQPPSSIKFTSVVDSPEVVLSKQ |
| | | NADILSDRKYREVWDTEKTNIHLNNDLPSLVLSKANAIAISNKLYTKDWDA |
| | | DKAKGYHIKEDAIAVLKARKSRDIISDYKYHEGYRKQVGHHIGARCVEEDPL |
| | | IMLALNSAKIASDALYKKDFNKSKTKFHLPVDMLTIELAKKCQVQVNDFNY |
| | | RTHLHNWTCLPDSTDVVQARKTYDLQSDAVYKADLEWLRGCGWNPHESV |
| | | EVLKVKHAQAVLADRGYRVKLDQQKFTAPVDRIDMVCAKNAAEILNEANY |
| | | RALWHTEKTKYTITDTPGLATAREVAKNLHPKLYTKDWDKVKATGFFMPG |
| | | DAVPIKQCIHNNKVQSDHKYKADYRKQAGHHIGARCVEDDPLIMLAINSAKI |
| | | ASDALYKKDFNKSKTKFHLPVDMLTIELAKKCQVQVNDDHYRTRLHNWTC |
| | | LPDSTDVTQARKAYDLQSDVVYKADLEWLRGCGWVAADCVDHVKVRNA |
| | | QKIMNERLYKKDAKDNFAKYTHIVDRPEIILATINAFNLSDLKYKETFNAEK |
| | | GKYIGSEDTPQLAHSREVAKVISEKYYKLAWDESKATGYQLDAHYIPVVGA |
| | | KKTREIVSDVKYHDTHEKAKGHYLAGTLVDFPEVMRCGEQEKQKGLRDYH |
| | | KEYNTNKAKNHIPADIIPNMVAKRCQDMLSDVLYRTLLHNWTCHPDQQDII |
| | | RARKNNEILSDVYYKDDLNWIKGIGCYAWDTPELVRAKNAHLLQSDLKYK |
| | | EEGKKEFNNYSIVTDTPVYVTALLGHTWASDLNYREAYHKDKHMYTTILDT |
| | | YNYAHNATLKDIYSTKTYTSAWDKIKAKDYTLPSDSHALKHAKQQKVILSN |
| | | VKYREDYEKFKSLYTLPKSLEDDPQTLRCLKAGKMGLDRLYKADYEKTKA |
| | | KNHIPPDMLDVLSARQTQNYVSEIGYRKHLHQWICMPDMQVYVQARKVNE |
| | | QLSDIFYKDDLNWLRGIGCYAWDTPEILRVKAADKLQSDNKYRAKGIEAFK |
| | | DYSVVIDTPTYETAKLSSQNLSDLHYRNDYATNVKGTNSSPAVTVDTERAR |
| | | LANFNQSDNLYKEANKNFMPTGYSLPHDNPLMKQAKQNSIVASSVKYKND |
| | | YEKSKATNYTMDPDGVSFVNIRNANKVTNPRLYRELYEKGKGKIHSTYDTP |
| | | EIRQVKMTQEAVSDLCYKEKYFANRGTMIPMGVTPQMIHCNHVNEITSDLR |
| | | YKEDLLWLRGIGCFLYDTPEMSTVRNITKFRSNYEVEAKKNHANFTVVLDT |
| | | PEYQRVTELKTHMSDLMYRAQGNVEKTKCTTTLDSLDVKRVKWAQQLSN |
| | | KYMYTDLASKERAHFTPEKDTPIMSHARAMKVVFSEIKYKDQYEKMKHKY |
| | | TSIHDTPILVRAKKAYLQSSDLRYKESFERSKGHYHSVKDALDIVCHRRVTD |
| | | DISEVKYREKYINSLGTWRSMPDRPEFFHSRIIGENVSDIKYKEDLDWLKGIG |
| | | CYVWDRPEMVQAEKNKVLFSERHYKETFEKNRHRFKYTCDTPFFQAAKKA |
| | | SVIINDRHYRAAYEKTKDKYSSVLDDPRNLLAKENAKNSQWKYREQYERA |
| | | KNKFTSILETPEHESHKRSKAIGDNVYKMDYNKNKAKGYTLGHDTPHSTHM |
| | | KKVKEITSVLKYKELYEKSKAQINMDPESFEIRAAKEAYKNISNLDYRKKYQ |
| | | ATKNKWIWTVDRPDFIQAAKANFQQSDIEYTYDKEMMKGCVISIVDDKLAV |
| | | LAKMNADLSNVKYREKFEKAKGQYKTVLDTPTNLHAKSVRILASESKYKLA |
| | | GKMEQQTGQFTTLPTTRDTIHAKDMGKLVSEKQYKQKFEKEKGKSKYNSM |
| | | RVPPDVQHAMDVAKNQSNFAYRQEAKAKLNYTSVAMRPDIMRATQASKL |
| | | ASNIGYREKARQEAAHGGSLVHRPDIALATAVSKLNSQVQYKAKYDKEKGL |
| | | RPKYDMKESVVYKTQKDAHQLASEVKYKGDLKKLHKPVTDMAESLSMQH |
| | | NLSTSKLSSDYQYKKKHEDSKGHYLMIADSPEQLHHKEASELQSHVKYKEK |
| | | YEKAKGKAMLDFETPTYVTAKEAQHMQSAKDYHKEYEESIKGKNLSGLEV |
| | | TPAMLHVRHATKIASERDYRRDLEEGVKGKGLTVFEETPELLRARNATAILS |
| | | QKEYKKTLETEIKGKGMLALATDTPDFMRARNATDILSQQKYKQTAEADRA |
| | | SYTSVIDTPDILHAQNIRNMVSQKKYREEAEKCMSHYVPVLDTPEMMRVRE |
| | | NQKNFSTIKYKDLLCRCTAISDLPEVKRVREMQKNISSVLYKDSSAKGTPQV |
| | | FTPEMERAKRNQEQISSVLYSDSFRKQVQGKAAFVLDTPEMRRVKETQRNIS |
| | | GVKYHQDFEKSKGSFTPTTSDPVMDRVKKNTQEFSDINYRGIQRRVVEMEK |
| | | RRVVEHEQETVTDLRVWRTNPGSVFDYDPAEDNIQSRSLHMMSVQAQRRS |
| | | KEHSRSTSAMSGVGDEKSEVSEDPAHHVSLYSNGFATSSMGYTHTKTVELQ |
| | | QRSSSVATQQTTVSSVPSHPSTTGKTVRAMYDYSSADMDEVSFKDGDVIVN |
| | | VQSIDEGWMYGTVQRSGKTGMLPANYVEAI |
| 214 | Salmon Q78BU2 | MCDDDETTALVCDNGSGLVKAGFAGDDAPRAVFPSIVGRPRHQGVMVGM |
| | (Actin alpha 1-1) | GQKDSYVGDEAQSKRGILTLKYPIEHGIITNWDDMEKIWHHTFYNELRVAPE |
| | | EHPTLLTEAPLNPKANREKMTQIMFETFNVPAMYVAIQAVLSLYASGRTTGI |
| | | VLDSGDGVTHNVPIYEGYALPHAIMRLDLAGRDLTDYLMKILTERGYSFVTT |
| | | AEREIVRDIKEKLCYVALDFENEMATAASSSSLEKSYELPDGQVITIGNERFR |
| | | CPETLFQPSFIGMESAGIHETAYNSIMKCDIDIRKDLYANNVLSGGTTMYPGI |
| | | ADRMQKEITALAPSTMKIKIIAPPERKYSVWIGGSILASLSTFQAMWITKQEY |
| | | DEAGPSIVHRKCF |
| 215 | Salmon ALBU2 | MQWLSVCSLLVLLSVLSRSQAQNQICTIFTEAKEDGFKSLILVGLAQNLPDST |
| | (Albumin 2) | LGDLVPLIAEALAMGVKCCSDTPPEDCERDVADLFQSAVCSSETLVEKNDLK |
| | | MCCEKTAAERTHCFVDHKAKIPRDLSLKAELPAADQCEDFKKDHKAFVGRF |
| | | IFKFSKSNPMLPPHVVLAIAKGYGEVLTTCCGEAEAQTCFDTKKATFQHAIA |
| | | KRVAELKSLCIVHKKYGDRVVKAKKLVQYSQKMPQASFQEMAGMVDKIV |
| | | ATVAPCCSGDMVTCMKERKTLVDEVCADESVLSRAAGLSACCKEDAVHRG |
| | | SCVEAMKPDPKPDGLSEHYDVHADIAAVCQTFTKTPDVAMGKLVYEISVRH |
| | | PESSQQVILRFAKEAEQALLQCCDMEDHAECVKTALAGSDIDKKITDETDYY |
| | | KKMCAAEAAVSDDNFEKSMMVYYTRIMPQASFDQLHMVSETVHDVLHAC |
| | | CKDEPGHFVLPCAEEKLTDAIDATCDDYDPSSINPHIAHCCNQSYSMRRHCIL |
| | | AIQPDTEFTPPELDASSFHMGPELCTKDSKDLLLSGKKLLYGVVRHKTTITED |
| | | HLKTISTKYHTMKDKCCAAEDQAACFTEEAPKLVSESAELVKV |
| 216 | Salmon ALBU1 | MQWLSVCSLLVLLSVLSRSQAQNQICTIFTEAKEDGFKSLILVGLAQNLPDST |
| | (Albumin 1) | LGDLVPLIAEALAMGVKCCSDTPPEDCERDVADLFQSAVCSSETLVEKNDLK |
| | | MCCEKTAAERTHCFVDHKAKIPRDLSLKAELPAADQCEDFKKDHKAFVGRF |
| | | IFKFSKSNPMLPPHVVLAIAKGYGEVLTTCCGEAEAQTCFDTKKATFQHAV |
| | | MKRVAELRSLCIVHKKYGDRVVKAKKLVQYSQKMPQASFQEMGGMVDKI |
| | | VATVAPCCSGDMVTCMKERKTLVDEVCADESVLSRAAGLSACCKEDAVHR |
| | | GSCVEAMKPDPKPDGLSEHYDIHADIAAVCQTFTKTPDVAMGKLVYEISVR |
| | | HPESSQQVILRFAKEAEQALLQCCDMEDHAECVKTALAGSDIDKKITDETDY |
| | | YKKMCAAEAAVSDDSFEKSMMVYYTRIMPQASFDQLHMVSETVHDVLHA |
| | | CCKDEQGHFVLPCAEEKLTDAIDATCDDYDPSSINPHIAHCCNQSYSMRRHC |
| | | ILAIQPDTEFTPPELDASSFHMGPELCTKDSKDLLLSGKKLLYGVVRHKTTIT |
| | | EDHLKTISTKYHTMKEKCCAAEDQAACFTEEAPKLVSESAELVKV |

An animal protein can comprise a cod protein (Table 13). A cod protein can comprise a cod muscle protein. A cod protein can comprise a cod blood protein. A cod protein can comprise a cod albumin protein. A cod protein can comprise a sequence that has at least or about 70%, at least or about 75%, at least or about 80%, at least or about 90%, at least or about 91%, at least or about 92%, at least or about 93%, at least or about 94%, at least or about 95%, at least or about 96%, at least or about 97% at least or about 98% at least or about 99% or about 100% sequence identity to any one of SEQ ID NO: 217-231.

A food product may comprise a fragment or a truncation of a cod protein. The food product may include a fragment or truncation of any one of SEQ ID Nos: 217-231. A sequence of the fragment or truncation may be identical or substantially identical to a portion of any one of SEQ ID Nos: 217-231. A sequence of the fragment or truncation may be at least about 70%, 80%, 90%, 95%, 98%, or more identical to a portion of a sequence of any one of SEQ ID Nos: 217-231. A length of the fragment or truncation may be at least about 10%, 15%, 20%, 30%, 40%, 50%, 60%, 70%, 80%, 90%, or more of a length of any one of SEQ ID Nos: 217-231. A length of the fragment or truncation may be less than or equal to about 90%, 80%, 70%, 60%, 50%, 40%, 30%, 20%, 15%, 10%, or less than a length of any one of SEQ ID Nos: 217-231.

TABLE 13
Cod Proteins

| SEQ | | |
|---|---|---|
| ID NO | Description | Amino Acid Sequence |
| 217 | Cod | MAKNCHNDYKMKFSGDEEFPDLSLHNNHMAKVLTKDIYNKLRGKSTPSGF |
| | A0A8C4Z9R4 | TVDDIIQTGVDNPGHPFIMTVGCVAGDEESYEVFKDLLDPIISDRHSGYKPT |
| | (Creatine kinase) | DKHRTDLNFENLKGGDDLDPAYVLSSRVRTGRSIKGFTLPPHNSRGERRTIE |
| | | KLSIEALATLSGEFKGKYYPLNGMTDKEQDQLINDHFLFDKPVSPLLTCAG |
| | | MARDWPDARGIWHNDAKTFLVWVNEEDHLRVISMQQGGNMKEVFRRFC |
| | | TGLLKIEETFKKHNHGFMWNEHLGYVLTCPSNLGTGLRGGVHVKLPKLST |
| | | HAKFEEILTRLRLQKRGTGGVDTASVGGVFDISNADRLGSSEVDQVQMVV |
| | | DGVKLMVAMEKKLEKSESIDDMIPAQK |
| 218 | Cod | FHKLLQTNGKGMIMLAYMTQEEGWDRDLLLDPAWEKQQRKTFTAWCNS |
| | A0A8C5D617 | HLRKAGTQIENIEEDFRNGLKLMLLLEVISGERLPKPDKGKMRFHKIANVN |
| | (Actinin alpha 3b) | KALDYICSKGVKLVSIGAEEIVDGNAKMTLGMIWTIILRFAIQDISVEETSAK |
| | | EGLLLWCQRKTAPYRNVNVQNFHISWKDGLALCALIHRHRPDLIDYSKLR |
| | | KDDPIGNLNTAFEVAEKFLDIPKMLDAEDIVNTPKPDEKAIMTYVSCFYHAF |
| | | AGAEQAETAANRICKVLAVNQENEKLMEEYEKLASELLEWIRRTIPWLENR |
| | | AAEQTMRAMQQKLEDFRDYRRVHKPPRVQEKCQLEINFNTLQTKLRLSNR |
| | | PAFMPSEGKMVSDIANAWKGLEQVEKGYEEWLLTEIRRLERLDHLAEKFK |
| | | QKCSLHETWTSGKEELLSQKDFESASLMEIRALMRKHEAFESDLAAHQDR |
| | | VEQIAAIAQELNELDYHDAVTVNARCQGICDQWDNLGTLTQKRRDSLERV |
| | | EKLWETIDQLYLEFAKRAAPFNNWMDGAMEDLQDMFIVHSIEEIQSLITAH |
| | | DQFKATLPEADKERMATMGIQSEILKIAQTYGIKLSGINPYTNLSPQDISNK |
| | | WDAVKHLVPLRDQMLQEEVARQQANERLRRQFAAQANIIGPWIQTKMEEI |
| | | GHVSADITGSLEDQMNNLKQYEQNIINYKSNIDKLEGDHQLSQESLIFDNKH |
| | | TNYTMEHIRVGWEQLLTTIARTINEVENQILTRDAKGISQEQLNEFRASFNH |
| | | FDRKRNGMMDPDDFRACLISMGYDLGEVEFARIMTLVDANNTGVVTFQAF |
| | | IDFMTRETAETDTAEQVMASFKILASDKSYITVDELRRELPLEQAEYCISRM |
| | | TRYVGADGTTGALDYISFSSALYGESDL |
| 219 | Cod | MAKPLTDQEKRKQISVRGLAGVENVTDIKSNFNRHLHYTLVKDRNVATKR |
| | A0A8C5D074 | DYYFALANTVRDHLVGRWIRTQQHYYEKDPKRVYYLSLEFYMGRTLQNT |
| | (Alpha-1,4 glucan | MVNLALENACDEATYQLGLDMEELQDIEEDAGLGNGGLGRLAACFLDSM |
| | phosphorylase) | ASLGLAAYGYGIRYEFGIFNQKIVDGWQVEEADDWLRYGNPWEKARPEY |
| | | MRPVHYYGRVEHTPGGVKWVDTQVVLALPYDTPVPGYRNNVVNTMRLW |
| | | SAKAPCDFNLKDFNVGGYIQAVLDRNLAENISRVLYPNDNFFEGKELRLKQ |
| | | EYFVVAATLQDIIRRFKSSKFGSTEFVRMDLSTLPDKVAIQLNDTHPALAIPE |
| | | LMRILMDLEELPWEKAWDIVVRTCAYTNHTVLPEALERWPIDLFQNLLPRH |
| | | LDIVYEINRRHLERVAQQFPGDEGRLRRMSLIEEGDHKKINMAHLCIVGSH |
| | | AVNGVARIHSEIIKDTVFKDFYEMDPEKFQNKTNGITPRRWLVMSNPGLAE |
| | | VIAERIGDDYIRDLDQLKNLLEFVDDDSLIRDVAKVKQENKMKFSAYLEEH |
| | | YKVKINPESMFDVQVKRIHEYKRQLLNCLHIITFYNRIKKQPNKAWTPRTIM |
| | | IGGKAAPGYHTAKMIIKLITAIGDIVNHDAVVGDRLKVIFLENYKVTLAEKII |
| | | PASDLSEQISTAGTEASGTGNMKFMLNGALTIGTMDGANVEMAEEAGEEN |
| | | LFIFGMRVDDVKAMDQTGYDAKSYYDRIPELKQAMDQISGGFFSPGQPDLF |
| | | KDIVNMLMNHDRFKVFADYEEYIKCQDKVSALYKDPKEWTKMVIRNIGSS |
| | | GKFSSDRTIAQYAREIWGMEPSLEKIPAPDDPRD |
| 220 | Cod | MPHAYPFLSTEQKKELSDIAKKIVAPGKGILAADESTGSVAKRFQGINAENT |
| | A0A8C5F6L1 | EENRRLYRQLLFTADDRVAPCIGGVIFFHETLYQKTDDGKLFPQLIRERGQV |
| | (Fructose- | VGIKVDKGVMPLAGTDGETTTQGLDGLYERCAQYKKDGADFAKWRCVLK |
| | bisphosphate | ITENTPSELAIMENANVLARYASICQMVRHHGIVPIVEPEILPDGDHDLQRTQ |
| | aldolase) | YVTEKVLAAVYKALSDHHVYLEGTLLKPNMVTAGHSCTRKYSAQEVAMA |
| | | TVTALRRTVPPAVPGVTFLSGGQSEEQASINLNAINQCPLGRPWALTFSYGR |
| | | ALQASALKAWGGKKENGKACQEEYIKRALNNNLASQGKYVSSGDKGAAG |
| | | GESLYVANHAY |
| 221 | Cod | MAPKKDVKAPAAAAKKAEPAKKAEPAPEPVAVPAPKTVDLSAVKVEFTPD |
| | A0A8C4YT70 | QMEDYREAFGLFDRVGDNKVCYNQIADIMRALGQNPTNKEVKAILGNPSD |
| | (Myosin, light | EDMNSKRVDFEGFLPMMQTIVNSPNKGTLDDYVEGLRVFDKEGNGTVMG |
| | chain 1, alkali; | AELRIVLSTLGEKMTEAEIDALMQGQEDENGCINYESFVKHIMSI |
| | skeletal, fast) | |
| 222 | Cod Q8AWX8 | MVKVGINGFGRIGRLVTRAGFQSKKVEIVAINDPFIDLEYMVYMFKYDSTH |
| | (Glyceraldehyde- | GRFHGEVKIEGDKLVIDGHKISVFHERDPANIKWGDAGAQYVVESTGVFTT |
| | 3-phosphate | IEKASAHLKGGAKRVIISAPSADAPMFVMGVNHEKYENSLQVVSNASCTTN |
| | dehydrogenase) | CLAPLAKVIHDNFGIVEGLMSTVHAITATQKTVDGPSGKLWRDGRGASQNI |
| | | IPASTGAAKAVGKVIPELNGKITGMAFRVPTPNVSVVDLTVRLEKPAKYDDI |
| | | KKVIKAASEGPMKGILGYTEDQVVSTDFNGDIRSSIFDAGAGIALNDHFVKL |
| | | VTWYDNEFGYSNRVIDLMAHMSCKE |
| 223 | Cod | MDAIKKKMQMLKLDKENALDRAEQAEGDKKAAEDRSKQLEDDLVALQK |
| | A0A8C5CXA2 | KLKGTEDELDKYSEALKDAQEKLELAEKKAADAESDVASLNRRIQLVEEEL |
| | (Tropomyosin 1, | DRAQERLATALTKLEEAEKAADESERGMKVIENRAMKDEEKMELQEIQLK |
| | alpha) | EAKHIAEEADRKYEEVARKLVIIESDLERTEERAELSEGKCSELEEELKTVT |
| | | NNLKSLEAQAEKYSQKEDKYEEEIKVLTDKLKEAETRAEFAERSVAKLEKT |
| | | IDDLEDELYAQKLKYKAISEELDHALNDMTSI |
| 224 | Cod | MPEAADAGELPTAEEDSGDEEGQGTELTGLFVERPQENVVIIAGADVVITA |
| | A0A8C5B6F3 | KVDSTTLTRKPTVKWLKGKWLDLASKAGKHLQFKETYDRETKIYTYEMK |
| | (Myosin binding | VIKAKVGAAGAAGAYRCEVTSKDKFDSASFEITVEAAQQSDDVDILSSFKR |
| | protein Cb) | ADAGEDDGNLDFSALLKAAKKKKRPEDDKPVIDVWELLKSAHPSEYEKIA |
| | | FEYGITDLRGMLKRLKKMKAEPKPSEAFLKRMESCYSVEKGKKIVLTCEVF |
| | | DPNVQVKWLKNGQEIKGSAKYIMETIGNIRKLTIMKTTLADDAAYECVVG |
| | | EDKCFTEVFVKEPPVTITKLMDDYHVVVGERVEFEVEVSEEGANAMWFFE |
| | | DIEIFKDTNPAKYRIKKDGRKHMLIILEAGLEDIGMYHCWTNGGHTKGELE |
| | | VEEKELLVLQDIADLTVKATDQAIFKCEVSDEKVTGKWFKDGVEVLPDER |
| | | HKMTHIGRFHKLLIDNVKPQDAGDYTFIPNGYALSLSAKLNFLEIKIDYVPR |
| | | QDPPKIHLDTTGNMVSQNTIIVVAGNKLRLDVEITGEPAPTVVWSKGEEVIT |
| | | ATEGRVRVDNRKTLSSFVIEGAEREDEANYTICVTNPAGEDKAKLFVKIVD |
| | | VPDPPQNVKCLSVGEDSSSMVWEPPAFDGGAPLKGYLMERKKKGSSRWT |
| | | KLNFDVYEATTYEAKRMIEGVYYEMRVFAINSIGMSQPSITSKPFMPLAPTS |
| | | EPTRLLVHDVTDGTCTLKWMAPERIGAGGLDGYIIEYCKEGETEWVVANK |
| | | ELCERQGYVVRGLPVGEKINFRVFAKNLAGVSPPATLAQPVTVREIVDLPKI |
| | | RLPRDLRTKYIRKVGDKINLVIPFQGKPRPVVKWYKDGAPVDEKIVGVRNS |
| | | TVDSILFIRSAERDHSGKYELVVQIENMEDRATIDLRVVERPGPPTKVKVTE |
| | | VWGFNAALEWEAPKDDGNCEITGYTIQKADKKTMEWYTIYEHNRRPSCTS |
| | | SDLIMGNEYMFRVYSENLVGPSEEPGQSKNTAVIGKTGLAYNPPEFKEKDV |
| | | SCIPTFTQPLLDRSVVAGYSTAMSCSVKGFPKPKIVWMKNKMILGGDPKYI |
| | | MLNNQGVLTLNIRKPSTFDGGRYSCMAVNDLGQALVECKLEIRIPTDPTAE |
| | | VK |
| 225 | Cod A0A8C5F718 | MAMRSGNYNHEYYHKQSKYVVKEYSSSESVVDDYQHKAAVRIQGVVRK |
| | (Myomesin 2b) | KMVEKYVREEPMIRGPKFVVRLRSHTVFENSPIKLFCTVEGFPVPIVTWYKD |
| | | GVILEHGSAGRYIVDDNVGMRSLEIPRCGTDDTAQYSAVASNIHGQASSQA |
| | | SIIVKRFRQEDESCLYAWLPYTVPMLPSIQYTKINITFTEKFGPVFASEGGSAS |
| | | LTATMHLEPNLANLQPDSQWYRDDTRLVDSKWVKINTGRGTSTLTIPNLFK |
| | | DDEGLYTLRLVTKGGTAEHSAFISVSDGPPPVVGAPGAPMDIRIHDANRDY |
| | | VIVSWKPPNTTTEGPILGYFVDKCEVGTENWAQCNDHPIKICKYPVPGLFEG |
| | | HSYYFRVRAVNARGISKPSRMSDPIAALDPTEFERLHALKLGGKLDVVTYD |
| | | DDMEAEGKPPGVSSGIYASETDRTYVVLSWKAPAYSSKAPMWYYIEKCQV |
| | | GTAAWQRVNTQVPIRSPRYAVFDLAEGKEYQFRVLSANMYGTSEPSEPSKL |
| | | IQTQALKGVPSAPGQVISTRETDTSLLIQWAPPKEPNNLIGYYIDQCVKGSK |
| | | DWTSANHKPHSSTKFVVSGLTTGETYVFRVQAINELGLSDESQESSPLSVKA |
| | | ALTSPTAPYDVALLGCDGQSMVLNWKKPLHTGGTAINAYFVDKRRSGAT |
| | | MWREVHIPAVTERVYQVEGLTQGAIYQFRVYAANLAGLGKSSKASADFSC |
| | | EAWTMPEPGPAYDLTASEVRDTSLVLEWKKPVYYGSGPITGYHVEYAKKG |
| | | SSDWTRANETAVSHRYLKVEGLEEGAKYVFRVRGVNEAGVGMASLPSDS |
| | | VVAKALAGAQEVSCSVDEKSGDIVFAFESCQMNVGSEFLWKKNYKEITEFS |
| | | SSIVVHTEGNLCKLMFKNAVKEDFGTYSVSVSHTDGVSSSCSISAEELQKM |
| | | LELSYSIRHPIIPLKSELAYKILERGRMRFWLQAEEISSKVTYKFFANNKELS |
| | | GADPNKMAHDVSTGVIEFILDHFTEDSEGTYTCQITDGGGKAQSSLVLIGDA |
| | | FKAALAEADYQRREYIRVKEGPHFSEFLTVKVGEDCSVTLVCKVANLKKES |
| | | EFHWYKEDTEIIPDVKPDLGSGVCKLPIHLFSRKTTGVYKATISDDRGKDMS |
| | | QVDISGKVFDDVINKINQLAGASAAELVINCTATGIQLQCHMKYYTSEMNI |
| | | HWMHGESKVGHSEKCVIGGTPAMATMEVIEPNDKDKGVYSIVITDLENTN |
| | | KRTLDLSGDVYDKAYAQFQKLKAEAYAEMNRGKVMGGLPDVVTIMEKK |
| | | TLSLTCTVCGDPKPLVSWLKNGNDVEPDNQYVVSLDQGKFACLTINGIGM |
| | | DDSGKYTMIVQNKYGGECVDIVVSVYRHGDTIPVAKPNLTAKTIIPPKMPIE |
| | | LPQPKTAAPSPAPPASPSPAPPKAVGGRGMKSPTPTRRK |
| 226 | Cod | MKSNPLTSNTGEHASYLLHVNGVMSITKIHAREILDSRGNPTVEVDLWTAK |
| | A0A8C5C5F8 | GLFRAAVPSGASTGIHEALELRDGDKTRYLGKGTQKAVDHVNKDISPKLIE |
| | (Phosphopyruvate | KNFSVVDQEKIDKFMLELDGTENKSKFGANAILGVSLAVCKAGAAEKGVP |
| | hydratase) | LYRHIADLAGNKDVILPVPAFNVINGGSHAGNKLAMQEFMILPVGAANFHE |
| | | AMRIGAEVYHNLKNVIKAKYGKDATNVGDEGGFAPNILENNEALELLKSAI |
| | | EKAGYPDKIIIGMDVAASEFYRSGKYDLDFKSPDDPARHISGKELGDLYKTF |
| | | IANYPVQSIEDPFDQDDWANWADFTAAVDIQVVGDDLTVTNPKRIQQAVE |
| | | KKACNCLLLKVNQIGSVTESIQACKLAQSNGWGVMVSHRSGETEDTFISDL |
| | | VVGLCTGQIKTGAPCRSERLSKYNQLMRIEEELGDKAKFAGKDYRHPKIN |
| 227 | Cod | MAPKKAKRRAGGGDSGSSNVFSMFEQSQIQEYKEAFTIIDQNRDGIISKDDL |
| | A0A8C4ZUF5 | RDVLASMGQINVKNEELEAMVKEASGPINFTVFLTMFGEKLKGADPEDVIV |
| | (Myosin light | AAFKILDPEATGSIKKEFLQELLTTQCDRFSPEEIKNMWSAFPPDVAGNVDY |
| | chain) | KNICYVITHGEEKEE |
| 228 | Cod | IMGDGEMQCFGEAAVYLRKPERERLEAQTSPFDAKTAFFVSEPKEMYLKG |
| | A0A8C5FJN9 | VLQSRDGGKATVKTLCDKTLTVKEEEIYPMNPPKYDKIEDMAMMTHLNEP |
| | (Myosin, heavy | TVLYNLKERYAAWMIYTYSGLFCVTVNPYKWLPVYDSSVVNAYRGKKRIE |
| | chain b) | APPHIFSISDNAYQAMLTDRENQSILITGESGAGKTVNTKRVIQYFATIAVSG |
| | | PKKTEQVAGKMQGSLEDQIIAANPLLEAYGNAKTVRNDNSSRFGKFIRIHF |
| | | ATSGKLASADIETYLLEKSRVTFQLSAERSYHIFYQLMTGHKPELIDALLITK |
| | | NPFDYHMISQGEITVKSIDDVEEFIATDDAIDILGFTADEKIGIYKLTGAVLH |
| | | HGNMKFKQKQREEQAEPDGNEEADKIGYLMGLNSADVLKALCYPRVKVG |
| | | NEYVTKGQTCPQVHNSVMALCKSVYEKMFLWMVVRINEMLDTKQPRQFF |
| | | IGVLDIAGFEIFNYNSLEQLCINFTNEKLQQFFNHHMFVLEQEEYKKEGIEW |
| | | EFIDFGMDLAACIELIEKPMGIFSILEEECMFPKASDTTFKNKLHDQHLGKTK |
| | | AFEKPKPAKGKAEAHFSLVHYAGTVDYNITGWLDKNKDPLNDSVIQLYQK |
| | | SSVKLLALLYASHALPDADAASKKGAKKKGGSFQTVSALFRENLGKLMTN |
| | | LRSTHPHFVRCLIPNESKTPGLMENFLVIHQLRCNGVLEGIRICRKGFPSRVL |
| | | YGDFKQRYKVLNASVIPEGQFIDNKKASEKLLGSIDVDSTQYKFGHTKVFF |
| | | KAGLLGALEEMRDEKLAALVTLTQALCRGFNMRKEFVKMMERREAVFTI |
| | | QYNIRSFMNVKTWPWMKVYFKIKPLLKSAETEKEMAQMKETFGKTKEDL |
| | | AKALSKKKELEEKMVSLLQEKNDLLLQVQSEAETLSDSEERCEGLIKSKIQL |
| | | EARLKETSERLEDEEEINSELTAKKRKLEDECSELKKDIDDLELTLAKVEKE |
| | | KHATENKVKNLTEEMSSQDEIIAKLTKEKKALQEAHQQTLDDLQAEEDKV |
| | | NTLSKAKCKLEQQVDDLEGSLEQEKKLRMDLERAKRKLEGDLKLAQESIM |
| | | DLENDKQQSDEKIKKRDFETSQLLNKIEDEQTLSAQFQKKIKELQARIEELE |
| | | EEIEAERAARAKVEKQRADLSRELEEISERLEEAGGATSLQIEMNKKREAEF |
| | | QKLRRDLEESTLQHEATSAALRKKQADSVAELGEQIDNLQRVKQKLEKEK |
| | | SECKMEIDDLSSNMEAVAKSKANLEKLARTLEDQLSELKSKNDEHVRQLN |
| | | DLSVORARLQTENGEFGRQQEEKEALISQLTRGKQAYTQQIEELKRHIEEEV |
| | | KAKNALAHSVQSARHDCDLLREQFEEEQEAKAELQRGMSKANSEVAQWR |
| | | SKYETDAIQRTEELEEAKKKLAQRLQDAEEAVEATNSKCASLEKTKQRLLG |
| | | EVEDLMIDVERANALAANLDKKQRNFDKVLAEWKQKYEESQAELEGAQK |
| | | EARSLSTEMFKLKNSYEESLDQLETLKRENKNLQQEISDLSEQMGETGKTIH |
| | | ELEKGKKTVEVEKSELQTSLEEAEATLEHEESKILRVQLELTQIKGEVDRKM |
| | | AEKDEEMEQIKRNSLRVIDSMQSNLDSEIRSRNDALRIKKKMEGDLNEMEI |
| | | QLSHANRQASEAQKQLRNVQGQLKDAQLHLDDALRGQEDMKEQVAMVE |
| | | RRNNLMLAEIEELRAALEQTERGRKVAEQELVDASERLGLLHSQNTSLINT |
| | | KKKLETDLIQIQGEVEDTVQEARNAEEKAKKAITDAAMMAEELKKEQDTS |
| | | SHLERMKKNLELTVKDLQHRLDEAESLAMKGGKKQLQKLEARVRELETEC |
| | | EAEQRRGSEAIKGVRKYERKVKELSYQCDEDKKNICRLQDLVDKLQLKMK |
| | | AYKRQAEESEEQANTHLSRYRKVQHELEEAQERADVSESQVNKLRTKSRN |
| | | GITKIIN |
| 229 | Cod | MATAGMPTRKVRKRVAVDTSKFMTPYLAHSMKMLEQYSHNMYRKAYDL |
| | A0A8C5F7Y0 | SRGTPPVHIADTPEMVRIRKAQEQLSEVKYRMEGNKARAGSLYDGEAKEIA |
| | (Nebulin) | HVKHMSELLSKVLYRQKWDETKDRYLLPPDAPELVLAVKNAANYSKKLY |
| | | TEDWDLEKASFLPYSDSPELRRVAKAQEALSDIQYKKGYDQRKLKFTSLAD |
| | | RPDVELAKKVFTQRSDLKYKEDYNKNVKGQWCETPYFDVATARVAMENL |
| | | SSRRYTQDWEAAKDQINFMQTETPVYDTNKKAGTSASSVKYRKDYEKTK |
| | | DKSDYNTLPATENPLLRQLRYAGTILSDKVYKKNYEKSRGFSINYCDTPKF |
| | | KMDSVLKKFTDTHYQDKYKNEVQGHYIGSYEDIYMLHCQKVEGFKSEQN |
| | | YKAEYEDIKTRCFFPQTITPEYEVDKKLAQCKDKVYRQHPDKVKFTQVTDS |
| | | PVQVQAALNAIQLSDINYKAKYEQTKTKNSLPADYPFFIQSRVNAFNLSENC |
| | | YKYNWEKEKAKRFEMKGDAISILAARAHTNIASDVKYKKEYEKNRGRMV |
| | | GALSIHDDPKILHSVHVAKIVSNREYTKDYEKVKTKYHTSLDMMSVTLAK |
| | | KSQAIASMTGYKKISNRYLMPYDAILVDLAKKANSIQSDNEYKSDYNQYTK |
| | | GSPWITFGSMDVELAKKAGEILSEKKYRKHPDTVPFTAIDDHPVMLQARVN |
| | | QLQRSDLFYKKGLEEIHQKYSLPIDMPELQLARANSYNISKNYYKAAWEET |
| | | ISKGYDLKLDAISIKAAKAARHAASDIQYKKAYEKARGHHVGFRNIKDDPL |
| | | LVHYMQVAKMQSEKNYKKDYHKSKLKYHSPVDMMSLVHAKQASKAQT |
| | | YAGYRKIQHHYSLMPDAMNLVLARKMNTDASDCEYKSEWNNYVRGTGW |
| | | VPIGSIGVETAKIGGQIQSENKYRTHPSTFKFSKLMDSMDLELATQAYTSAW |
| | | EKDKRTIHVMPDSPEIVLAKANAITMSDVREYYYKLHQNGYHLKGDAIPIV |
| | | AAKLGTKIASNYKYKLDYEKARGHHVGFRNIKDDPLLVHYMQVAKMQSE |
| | | KNYKKDYEKAKLKYHSPVDMMSLVHAKEASKAQTYAGYRKIPHTYFLLP |
| | | DNMQLQLCRKMQEIQSDNEYKSEYNNLYKGTGWVPIGSIDVELAKTANSA |
| | | LREHGYRQHPSSFKFTSKTDSMDMVLALANSKIIDQAAYKASGEKVKHTY |
| | | HLPADAPEFIQAKFNAQTMSKSHYKQKWVEHIAKGYDMRPDAISILHAKQ |
| | | GRHIASDIQYKKAYEKARGHHVGFRSIKDDPLLVHYMQVAKMQSEKNYK |
| | | KDYEKAKLKYHSPVDMMSLVHAKEASKAQTYAGYRKIQHHYNLMPDAM |
| | | NLVLARKMNTDASDCEYKSEWNNYVRGTGWVPIGSIGVETAKIGGEIQSE |
| | | NKYRTHPSTFKFSKLMDSMDLELATANNKIMNKDKRTIHVMPDSPEIVLAK |
| | | ANAITMSDKLYKAGVMEMAKKGYHLKGDAIPIVAAKLGTKIASNYKYKL |
| | | DYEKARGHHVGFRNIKDDPLLVHYMQVAKMQSERNYTKDYQKSKLKYHS |
| | | PVDMMSLVHAKEASKAQTYAGYRKIEHHYNLMPDAMNLVLARKMNTDA |
| | | SDCEYKSEWNNYVRGTGWVPIGSIGVETAKIGGEIQSENKYRTHPSTFKFSK |
| | | LMDSMDLELATANNKIMNKQAYTSAWEKDKRTIHVMPDSPEIVLAKANAI |
| | | TMSDKLYKAGVMEMAKKGYHLKGDAIPIVAAKLGTKIASNYKYKLDYEK |
| | | ARGHHVGFRCIQDDPLLVHYMQLAKQQSEKNYKKDYEKAKLKYHSPVDM |
| | | MSLVHAKDATKAQTYAGYRKIQHHYSLLPDAMNLVLARNMQVNISDNQY |
| | | KSDYESYCKGIGWIPIGSLDVEKAKIAGKIQSEKLYRQHPSNLKFTKDMFSM |
| | | DLTLATANNQIMNKVLLALYCVDYTLMNILSSGNNLTNNFVVSFSKLYKL |
| | | DNELSKKKGHNVRKDAIAILAAKNCTTIASDYKYKAGYRKQVGHHIGARC |
| | | VEDDPLLVLALNSARIASDALYKKDFNKSKTKFNLPVDMMAFELAKKNQI |
| | | QVNHANYITRLHNWTCLPDSTDVVNARRAYEIQSDNYYKEDQKLMQGIG |
| | | WVPIGSLDVEKAKKAGEILSEHLYRKHPSNYKFTSKCDAMPLVLAKANAQI |
| | | TNKKAYHDAWDKEKSNIHIMPDAMDVVLAKANRANYSIKRYKQANEDAK |
| | | KKGYDLRNDAIGIIAARAQRNIISDIKYKAGYRKQVGHHIGARCVEDDPLL |
| | | MLALNSARIASDYLYKKDFNKSKTKFNLPVDMMAFELAKKNQIQVNHAN |
| | | YITRLHNWTCLPDSTDVVNARRAYEIQSDNYYKEDQKLMQGIGWVPIGSL |
| | | DVEKAKKAGEILSEHLYRKHPSNYKFTSKCDAMPLVLAKANAQITNKKAY |
| | | HDAWDKEKSNIHIMPDAMDVVLAKANRANYSIKGYKQANEDAKKKGYD |
| | | LRNDAIGIIAARAQRNIISDIKYKAGYRKQVGHHIGARCVEDDPLLVLALNS |
| | | ARIASDYLYKKDFNKSKTKFNLPVDMMAFELAKKNQIQVNHANYITRLHN |
| | | WTCLPDSTDVVNARRAYEIQSDNYYKEDQKLMQGIGWVPIGSLDVEKAKK |
| | | AGQILSEHLYRKHPSNYKFTSKCDAMPLVLAKANAQITNKKAYHEAWDKE |
| | | KSNIHIMPDAMDVVLAKANRANYSIKKYKEANEESKKKGYDLRNDAISIIA |
| | | ARAQRNIVSDYKYKAGYRKQTGHHIGARCVEDDPLLVLALNSARIASDAL |
| | | YKKDFNKSKTKFNLPVDMMAFELAKKNQIQVNHANYITRLHNWTCLPDST |
| | | DVVHARHVYEIQSDAVYKADLKWLQGMGWVPIGSLDVEKTKKAGEILSE |
| | | RKYRQPASSHKFTSPLDAMEIVLAKNNALTMNQRLYTEAWEKDKTKLHIT |
| | | SETPEIILSQQNAINMSNKKYKQGYEDTIKKGYYLPGDAISVKSAKHSRNIIS |
| | | DYKYKAGYRTQVGHHIGARCVEDDPLLMLALNSARIASDYLYKKDFNKSK |
| | | TKFNLPVDMMAFELAKKNQIQVNHANYITRLHNWTCLPDSTDVVNARKA |
| | | YDLQSDAVYKADMEFMKGIGWVPIGSLDVVKAKHAGKILSDHLYKQKTD |
| | | KLKYTSDTKAMPMLLAKANAEVMNKKSYVAAWEKDKIKTHLIPDLPEILL |
| | | SRSNAYNLSNKMYKEDVVKMFRKGYHLKGDAIAIVAAKKGREIISDIKYK |
| | | AGYRKQVGHHIGALSVRDDPLIMLALNSARIASDALYKKDFNQSKTKFHLP |
| | | VDMLTIELAKKCQVQVNDFNYRTRLHNWTCLPDSTDVVQARKAYDLQSD |
| | | AVYKADMEWIKGQGWVPIGSLDVEKAKKAAEILSERKYREHPSKFKFTAK |
| | | TDDMPYALAKANAEIMNKQSYVAAWESDKTKLHIMPDTPEIILGQQNKLN |
| | | NSRKYYREAYEETLKKGYYLPKDAISVLAARASRNIISDYKYKAGFRKQCG |
| | | HHIGALSVQDDPLLMMAANAAKIFSANLYKQDFHKTKTKYKLPVDMVAF |
| | | DLAKTAQSHMKEVTYRTRLHNWTCMPDNTDVVHARKVYDLHSDAVYRA |
| | | DMEWIRGCGWSPQDSVDVVKARNAQKILNENLYRQAPSTVKFTSVVDLPA |
| | | IVLSKQNAENISEMKYTAAWNKDKLTFHLKPDIPSVQLAKANAINISNKLYT |
| | | KAWDNQKAKAYHIKEDTIAVLKARASRDIISDYKYKTGYRKQVGHHIGAL |
| | | SVRDDPLIMLALNSARIASDALYKKDFNQSKTKFHLPVDMLTIELAKKCQIQ |
| | | VNDFNYRTRLHNWTCLPDSTDVVQARKAYDLQSDAVYKADMEWIRGCG |
| | | WVPHECVDVVKVKHAQKILADRGYRVKLDTQKFTAPVDRMDFVCAKNA |
| | | AEVLNDAKYREAWHTDKSKFSLVDTPGLATSREVAKIMHPKLYTKDWDK |
| | | VKATGYFMPGDAVPIKQCTQTTKVQSSHKYLEGYRKQVGHHIGALSVFDD |
| | | PLIMLAMNSAKIASEALYKKDFNKSKTKFHLPVDMLTIELAKKCQIQVNDF |
| | | NYRTRLHNWTCLPDSTDVVNARKAYDLQSDFVYKADLEWIRGCGWVPAE |
| | | SVHHVKVRKAQEILNERLYKKGAIENFSKYTHVVDRPELILARVAAANLSD |
| | | LKYKETFNNEKGHYIGSEDTPQLAHSRECAKNISEAKYHESHEKAKGHYM |
| | | ANTLVDFPEVVRCGEQEKAKALRVYQKEYHESKTNNHIPSDIIANQVAKRC |
| | | QDMLSDVIYRTHLHQWTCPPDQLDTVRARKGNEILSDVFYKEDLTWMKG |
| | | MGCYVWDTPEIVRAKKSYELQSELKYKEAGKKEFNNYSIVTDTPVYVTAIL |
| | | GSTWASELNYREAYHKEKHLYTTILDTHDYARCHDFKHFFSNKIYTAAWD |
| | | KIKAKGYKIPHDSHAMMHAKQQKVILSGVKYKEDYEKFKSLYSLPKCLQD |
| | | DPATARCIKAGKLNLDRLYKEDYEKTKAKNHIPADMLDIVSGREVQRNVS |
| | | EINYRKRLHQWVCLPDMQVFVQARKVNDQLSDIFYKDDLNYLRGIGCYA |
| | | WDTPEILRVKTAGDQQSENKYKAKAVEAYKDYTVVLDTPTYETAKQNAV |
| | | NLSDLHYRLDYNANVKGTNTAPAVTMETERARLANYNQSDNLYKEANKN |
| | | SMATGYLMTNDTPVVKQVKNNSLVASSVKYKAAYEMMKATGYTLHPEG |
| | | VTFVNTRKVNQVINPRLYREQYEKDKDKIHSTLDTPDIRQVKMNQEHLSEL |
| | | CYREKYNNTKGQLINMPMTPQLMHCQHVNQITSDLRYKEDLLWLRGLGCF |
| | | MYDTPEMVNVRNITKFKNNYQVEAKKNHSKFSVVLDTPEYQRVTELKSH |
| | | MSMVSPTSNTSLNTLDSVDIQRSKWAQQLSNKFLYTDLASKERAHFSQEVE |
| | | TPAMGLARTMKVTNSYNKYKEQYEKMKHRYTSIADTPILIRAKKAYLQSS |
| | | DLRYKETFERSKGHYHTVKDALDILCHRRVTDDISEVKYREKYINSLGTWK |
| | | SIPDRPEFFFNRIVGENVSSVKYKEDLEWMKGIGCFVWDTPEMVQADINKT |
| | | LYSGRLYKASFEKNRYNFKYTCDTPFFHAAKNASSLINERSYRSSYEKNKD |
| | | KFTITTDDPRYQLARENNKMSQAKYKSRAKELLKSGCNELHRPDILTALYQ |
| | | SVLGSKWKYREQYERVKSKFTSILETPEYESHKRSKKIGDIVYRMEYNKNK |
| | | AKGYSLPYDTPHQNHMKKVQALTSNLKYREVYEKAKSQINMDPDAFEIRA |
| | | AKEAYKNTTNLDYKKKYEATKNKWIWTVDRPDFISHSRNTLQQSDVEYKY |
| | | DKEMMKGCVIPIVDDKITLLAKKGCEMASDIKYKEKYEKAKGHYNPVADS |
| | | PQILHAKAVRTLASESKYKEASKKEMQSGSFTTLPETRDTVHSKEVNKLISG |
| | | KLYKSKFEKEKGQSKYNRMIVPPDVQHAMDVAKSQSSYAYKRDAKDNLH |
| | | YTTTADRPDIRKATQAAKLISDVGYREKARAEASRGGSLTYRPDIALATEVS |
| | | KLTSLVDAKSALHGAASYDTLQMRHSKKMSALTSNVKYHEKFDKEMKGK |
| | | KPKYDLKDSKIYQTLKDATNLASEVKYKGDLKKLHKPVTDMAESLSMQH |
| | | NLSTSKLSSDYRYKKKFKDSKGHYLVIADTPEQLHHKEATELQSSVSGPSLC |
| | | GVHVCKDYKKDYEEGMRGKNLCGLEVTPAMIHVRHATKIASEREYRKDL |
| | | EEGVKGKGLSAPEETPEHLRAKNATHILSEREYKKSLELEIKGKGMLALAT |
| | | DTPDFMRARNATDILSQTKYKHTAELDRASFTSVIDTPDIIHAQQMRNIISQN |
| | | KYKAEAGKTMSHYVPVFDTPEMQRVRENQKNFSTLQYQADHKIIKGKVLT |
| | | VKDTPEMLRVKENTKNFSSVLYKDSKAKGTPVVFTPEMERVKRNQEHISSV |
| | | LYSDSFRKQVQGKAAFVLDTPEMRRVKESQLNISGVRYHQDHEKSKGSFT |
| | | AMASYPEMERVKKNTQEFSDINYRGIQRRVVEMERRRVVEHEQETTTDLR |
| | | VWRTNPGSVFDYDPAEDNIQSRSLHMMSVHAQRRSKEHSRSTSAMSGPAD |
| | | EKSEMSHEGDHHMSTYSNGFGASSVGYQTTKTVELQQRSSSVATQQTTVS |
| | | SIPSHPSTTGKTVRAMYDYGAADNDEVTFKDGDVIVNVQSIDEGWMYGTV |
| | | QRTGKTGMLPANYVEAV |
| 230 | Cod | MSTDAEMAVYGQAAIYLRKPEKERLEAQSKPFDAKSAAYVTDVKELYLKC |
| | A0A8C5ARQ5 | TILKKEGGKVTVKVHDTEEEKTVKDDDVTPMNPPKYDKIEDMAMMTHLN |
| | (Myosin heavy | EASVLYNLKERYAAWMIYTYSGLFCATVNPYKWLPVYDQHVVCAYRGKK |
| | chain, fast skeletal | RMEAPPHIFSVSDNAYQFMLTDRENQSVLITGESGAGKTVNTKRVIQYFATI |
| | muscle-like) | SVGGDKKKDIGAGKIQGSLEDQIIAANPLLEAYGNAKTVRNDNSSRFGKFIR |
| | | IHFGTSGKLSSADIETYLLEKSRVTFQLPAERGYHIFYQMMTNHKPEIIEMTL |
| | | ITTNPYDFPTCSMGQIAVASIDDKEELVATDTAIDILGFNNDEKMGIYKLTG |
| | | AVLHHGNMKFKQKQREEQAEPEGTEDADKVAYLLGLNSADMLKGLCYPR |
| | | VKVGNEYVTKGQTVPQVLNSVSALAKSIYERMFLWMVIRINQMLDTKQPR |
| | | SSFIGVLDIAGFEIFDYNSMEQLCINFTNEKLQQFFNHHMFVLEQEEYKKEGI |
| | | IWEFIDFGMDLAACIELIEKPMGIFSILEEECMFPKASDVTFKAKLYDQHLGK |
| | | TKAFEKPKPAKGKAEAHFSLVHYAGTVDYNITGWLDKNKDPLNDSVVQL |
| | | YQKSGVKLLPVLYPPVASEDSTKKGGKKKGGSMQTVSSQFRENLGKLMTN |
| | | LRSTHPHFVRCLIPNESKTPGLMENFLVIHQLRCNGVLEGIRICRKGFPSRILY |
| | | ADFKQRYKVLNASVIPEGQFIDNKKASEKLLGSIDVNHEEYKFGHTKVFFK |
| | | AGLLGTLEEMRDEKLATLVGMIQALCRGFQMRKEFKKMMERRHAVYTIQ |
| | | YNIRSFMNVKTWPWMKVYYKIKPLLKSAETEKELSAMKENYEKMKKDLA |
| | | TALAKKKELEEKMVSLLQEKNDLSLQVASEGENLNDAEERCEGLIKSKIQL |
| | | EARLKETTERLEDEEEMNADLTAKKRKLEDECSELKKDIDDLELTLAKVEK |
| | | EKHATENKVKNLTEEMASQDESVAKLSKEKKALQEAHQQTLDDLQAEED |
| | | KVNTLSKAKTKLEQQVDDLEGSLEQEKKLRMDLERSKRKLEGDLKLAQES |
| | | IMDLENDKQQSDEKIKKKDFETSQLLSKIEDEQSLAIQLQKKIKELQARIEEL |
| | | EEEIEAERAARAKVEKQRADLSRELEEISERLEEAGGATSAQIEMNKKREAE |
| | | FQKLRRDLEESTLQHEATAAALRKKQADSVAELGEQIDNLQRVKQKLEKE |
| | | KSEYKMEIDDLSSNMETVAKAKGNLEKICRTLEDQLSEIKAKSDENSRQIND |
| | | IGAQRARLLTENGEFGRQLEEKEALVSQLTRGKQAYTQQIEELKRQIEEEVK |
| | | AKNALAHGVQSARHDCDLLREQFEEEQEAKAELQRGMSKANGEVAQWRS |
| | | KYETDAIQRTEELEESKKKLAQRLQEAEEQIEAVNSKCASLEKTKQRLQGE |
| | | VEDLMVDVERANGLAANLDKKQRNFDKVLAEWKQKYEEGQAELEGTQK |
| | | EARSLSTELFKMKNSYEEALDQLETLKRENKNLQQEISDLSEQIGETGKSIH |
| | | ELEKSKKQVETEKTEIQTALEEAEGTLEHEESKILRVQLELNQIKGEVDRKL |
| | | AEKDEEMEQIKRNSLRITDSMQSTLDSEVRSRNDALRIKKKMEGDLNEMEI |
| | | QLSHANRQAAEAQKQLRNVQGQLKDAQLHLDDAVRGGEDLKEQAAMVD |
| | | RRNGLMVAEIEELRAALEQTERGRKVAEQELIDASERVGLLHSQNTSLLNT |
| | | KKKLETDLVQVQGEVDDTVQEARNAEEKAKKAITDAAMMAEELKKEQDT |
| | | SSHLERMKKNLEVTVKDLQHRLDEAENLAMKGGKKQLQKLESRVRELETE |
| | | VEGEQRRGVDAVKGVRKYERRVKELTYQTEEDKKNGARLQDLVDKLQLK |
| | | VKAYKRQAEEAEEQANSYLSKCRKVQHELEEAEERADIAETQVNKLRSKS |
| | | RDGGKGKDPAE |
| 231 | Cod Q78AY8 | MCDDEETTALVCDNGSGLVKAGFAGDDAPRAVFPSIVGRPRHQGVMVGM |
| | (Actin alpha 1) | GQKDSYVGDEAQSKRGILTLKYPIEHGIITNWDDMEKIWHHTFYNELRVAP |
| | | EEHPTLLTEAPLNPKANREKMTQIMFETFNVPAMYVAIQAVLSLYASGRTT |
| | | GIVLDSGDGVTHNVPVYEGYALPHAIMRLDLAGRDLTDYLMKILTERGYSF |
| | | VTTAEREIVRDIKEKLCYVALDFENEMATAASSSSLEKSYELPDGQVITIGN |
| | | ERFRCPETLFQPSFIGMESAGIHETAYNSIMKCDIDIRKDLYANNVLSGGTT |
| | | MYPGIADRMQKEITALAPSTMKIKIIAPPERKYSVWIGGSILASLSTFQQMWI |
| | | SKQEYDEAGPSIVHRKCF |

An animal protein can comprise a tuna protein (Table 14). A tuna protein can comprise a tuna muscle protein. A tuna protein can comprise a tuna blood protein. A tuna protein can comprise a tuna albumin protein. A tuna protein can comprise a sequence that has at least or about 70%, at least or about 75%, at least or about 80%, at least or about 90%, at least or about 91%, at least or about 92%, at least or about 93%, at least or about 94%, at least or about 95%, at least or about 96%, at least or about 97% at least or about 98% at least or about 99% or about 100% sequence identity to SEQ ID NO: 232.

A food product may comprise a fragment or a truncation of a tuna protein. The food product may include a fragment or truncation of any one of SEQ ID Nos: 232. A sequence of the fragment or truncation may be identical or substantially identical to a portion of any one of SEQ ID Nos: 232. A sequence of the fragment or truncation may be at least about 70%, 80%, 90%, 95%, 98%, or more identical to a portion of a sequence of any one of SEQ ID Nos: 232. A length of the fragment or truncation that is at least about 10%, 15%, 20%, 30%, 40%, 50%, 60%, 70%, 80%, 90%, or more of a length of any one of SEQ ID Nos: 232. A length of the fragment or truncation may be less than or equal to about 90%, 80%, 70%, 60%, 50%, 40%, 30%, 20%, 15%, 10%, or less than a length of any one of SEQ ID Nos: 232.

TABLE 14
Tuna Proteins

| SEQ | | |
|---|---|---|
| ID NO | Description | Amino Acid Sequence |
| 232 | Tuna | MCDDDETTALVCDNGSGLVKAGFAGDDA |
| | XP_042264497.1 | PRAVFPSIVGRPRHQGVMVGMGQKDSYV |
| | (actin alpha 1, | GDEAQSKRGILTLKYPIEHGIITNWDDM |
| | skeletal | EKIWHHTFYNELRVAPEEHPTLLTEAPL |
| | muscle b) | NPKANREKMTQIMFETFNVPAMYVAIQA |
| | | VLSLYASGRTTGIVLDAGDGVTHNVPVY |
| | | EGYALPHAIMRLDLAGRDLTDYLMKILT |
| | | ERGYSFVTTAEREIVRDIKEKLCYVALD |
| | | FENEMATAASSSSLEKSYELPDGQVITI |
| | | GNERFRCPETLFQPSFIGMESAGIHETA |
| | | YNSIMKCDIDIRKDLYANNVLSGGTTMY |
| | | PGIADRMQKEITALAPSTMKIKIIAPPE |
| | | RKYSVWIGGSILASLSTFQQMWISKQEY |
| | | DEAGPSIVHRKCF |

An additional gene that is not a gene of interest can be a gene that modulates protein expression. A gene that modulates protein expression can enhance protein expression, decrease protein expression, stop protein expression, or maintain protein expression.

A gene that modulates protein expression can increase protein expression by at least about 5%, at least about 10%, at least about 20%, at least about 30%, at least about 40%, at least about 50%, at least about 60%, at least about 70%, at least about 80%, at least about 90%, at least about 100%, at least about 150%, at least about 200%, at least about 300%, at least about 400%, at least about 500%, at least about 600%, at least about 700%, at least about 800%, at least about 900% at least about 1,000%, at least about 5,000%, at least about 10,000% or more. A gene that modulates protein expression can increase protein expression by at most about 10,000%, at most about 5,000%, at most about 1,000%, at most about 900%, at most about 800%, at most about 700%, at most about 600%, at most about 500%, at most about 400%, at most about 300%, at most about 200%, at most about 150%, at most about 100%, at most about 90%, at most about 80%, at most about 70%, at most about 60%, at most about 50%, at most about 40%, at most about 30%, at most about 20%, at most about 10%, at most about 5%, or less.

A gene that modulates protein expression can increase protein expression by at least or up to about 1-fold, at least or up to about 2-fold, at least or up to about 3-fold, at least or up to about 4-fold, at least or up to about 5-fold, at least or up to about 6-fold, at least or up to about 7-fold, at least or up to about 8-fold, at least or up to about 9-fold, at least or up to about 10-fold, at least or up to about 20-fold, at least or up to about 30-fold, at least or up to about 40-fold, at least or up to about 50-fold, at least or up to about 60-fold, at least or up to about 70-fold, at least or up to about 80-fold, at least or up to about 90-fold, at least or up to about 100-fold, at least or up to about 500-fold, at least or up to about 1,000-fold, at least or up to about 5,000-fold, or at least or up to about 10,000-fold. A gene that modulates protein expression can increase protein expression by at most or less than about 10,000-fold, at most or less than about 5,000-fold, at most or less than about 1,000-fold, at most or less than about 500-fold, at most or less than about 100-fold, at most or less than about 90-fold, at most or less than about 80-fold, at most or less than about 70-fold, at most or less than about 60-fold, at most or less than about 50-fold, at most or less than about 40-fold, at most or less than about 30-fold, at most or less than about 20-fold, at most or less than about 10-fold, at most or less than about 9-fold, at most or less than about 8-fold, at most or less than about 7-fold, at most or less than about 6-fold, at most or less than about 5-fold, at most or less than about 4-fold, at most or less than about 3-fold, at most or less than about 2-fold, or at most or less than about 1-fold.

A gene that modulates protein expression can decrease protein expression by at least about 5%, at least about 10%, at least about 20%, at least about 30%, at least about 40%, at least about 50%, at least about 60%, at least about 70%, at least about 80%, at least about 90%, at least about 100%, at least about 150%, at least about 200%, at least about 300%, at least about 400%, at least about 500%, at least about 600%, at least about 700%, at least about 800%, at least about 900% at least about 1,000%, at least about 5,000%, at least about 10,000% or more. A gene that modulates protein expression can decrease protein expression by at most about 10,000%, at most about 5,000%, at most about 1,000%, at most about 900%, at most about 800%, at most about 700%, at most about 600%, at most about 500%, at most about 400%, at most about 300%, at most about 200%, at most about 150%, at most about 100%, at most about 90%, at most about 80%, at most about 70%, at most about 60%, at most about 50%, at most about 40%, at most about 30%, at most about 20%, at most about 10%, at most about 5%, or less.

A gene that modulates protein expression can decrease protein expression by at least or up to about 1-fold, at least or up to about 2-fold, at least or up to about 3-fold, at least or up to about 4-fold, at least or up to about 5-fold, at least or up to about 6-fold, at least or up to about 7-fold, at least or up to about 8-fold, at least or up to about 9-fold, at least or up to about 10-fold, at least or up to about 20-fold, at least or up to about 30-fold, at least or up to about 40-fold, at least or up to about 50-fold, at least or up to about 60-fold, at least or up to about 70-fold, at least or up to about 80-fold, at least or up to about 90-fold, at least or up to about 100-fold, at least or up to about 500-fold, at least or up to about 1,000-fold, at least or up to about 5,000-fold, or at least or up to about 10,000-fold. A gene that modulates protein expression can decrease protein expression by at most or less than about 10,000-fold, at most or less than about 5,000-fold, at most or less than about 1,000-fold, at most or less than about 500-fold, at most or less than about 100-fold, at most or less than about 90-fold, at most or less than about 80-fold, at most or less than about 70-fold, at most or less than about 60-fold, at most or less than about 50-fold, at most or less than about 40-fold, at most or less than about 30-fold, at most or less than about 20-fold, at most or less than about 10-fold, at most or less than about 9-fold, at most or less than about 8-fold, at most or less than about 7-fold, at most or less than about 6-fold, at most or less than about 5-fold, at most or less than about 4-fold, at most or less than about 3-fold, at most or less than about 2-fold, or at most or less than about 1-fold.

An additional gene of interest can be a gene that modulates protein secretion. A gene that modulates protein secretion can enhance protein secretion, decrease protein secretion, stop protein secretion, or maintain protein secretion.

A gene that modulates protein secretion can increase protein secretion by at least about 5%, at least about 10%, at least about 20%, at least about 30%, at least about 40%, at least about 50%, at least about 60%, at least about 70%, at least about 80%, at least about 90%, at least about 100%, at least about 150%, at least about 200%, at least about 300%, at least about 400%, at least about 500%, at least about 600%, at least about 700%, at least about 800%, at least about 900% at least about 1,000%, at least about 5,000%, at least about 10,000% or more. A gene that modulates protein secretion can increase protein secretion by at most about 10,000%, at most about 5,000%, at most about 1,000%, at most about 900%, at most about 800%, at most about 700%, at most about 600%, at most about 500%, at most about 400%, at most about 300%, at most about 200%, at most about 150%, at most about 100%, at most about 90%, at most about 80%, at most about 70%, at most about 60%, at most about 50%, at most about 40%, at most about 30%, at most about 20%, at most about 10%, at most about 5%, or less.

A gene that modulates protein secretion can increase protein secretion by at least or up to about 1-fold, at least or up to about 2-fold, at least or up to about 3-fold, at least or up to about 4-fold, at least or up to about 5-fold, at least or up to about 6-fold, at least or up to about 7-fold, at least or up to about 8-fold, at least or up to about 9-fold, at least or up to about 10-fold, at least or up to about 20-fold, at least or up to about 30-fold, at least or up to about 40-fold, at least or up to about 50-fold, at least or up to about 60-fold, at least or up to about 70-fold, at least or up to about 80-fold, at least or up to about 90-fold, at least or up to about 100-fold, at least or up to about 500-fold, at least or up to about 1,000-fold, at least or up to about 5,000-fold, or at least or up to about 10,000-fold. A gene that modulates protein secretion can increase protein secretion by at most or less than about 10,000-fold, at most or less than about 5,000-fold, at most or less than about 1,000-fold, at most or less than about 500-fold, at most or less than about 100-fold, at most or less than about 90-fold, at most or less than about 80-fold, at most or less than about 70-fold, at most or less than about 60-fold, at most or less than about 50-fold, at most or less than about 40-fold, at most or less than about 30-fold, at most or less than about 20-fold, at most or less than about 10-fold, at most or less than about 9-fold, at most or less than about 8-fold, at most or less than about 7-fold, at most or less than about 6-fold, at most or less than about 5-fold, at most or less than about 4-fold, at most or less than about 3-fold, at most or less than about 2-fold, or at most or less than about 1-fold.

A gene that modulates protein secretion can decrease protein secretion by at least about 5%, at least about 10%, at least about 20%, at least about 30%, at least about 40%, at least about 50%, at least about 60%, at least about 70%, at least about 80%, at least about 90%, at least about 100%, at least about 150%, at least about 200%, at least about 300%, at least about 400%, at least about 500%, at least about 600%, at least about 700%, at least about 800%, at least about 900% at least about 1,000%, at least about 5,000%, at least about 10,000% or more. A gene that modulates protein secretion can decrease protein secretion by at most about 10,000%, at most about 5,000%, at most about 1,000%, at most about 900%, at most about 800%, at most about 700%, at most about 600%, at most about 500%, at most about 400%, at most about 300%, at most about 200%, at most about 150%, at most about 100%, at most about 90%, at most about 80%, at most about 70%, at most about 60%, at most about 50%, at most about 40%, at most about 30%, at most about 20%, at most about 10%, at most about 5%, or less.

A gene that modulates protein secretion can decrease protein secretion by at least or up to about 1-fold, at least or up to about 2-fold, at least or up to about 3-fold, at least or up to about 4-fold, at least or up to about 5-fold, at least or up to about 6-fold, at least or up to about 7-fold, at least or up to about 8-fold, at least or up to about 9-fold, at least or up to about 10-fold, at least or up to about 20-fold, at least or up to about 30-fold, at least or up to about 40-fold, at least or up to about 50-fold, at least or up to about 60-fold, at least or up to about 70-fold, at least or up to about 80-fold, at least or up to about 90-fold, at least or up to about 100-fold, at least or up to about 500-fold, at least or up to about 1,000-fold, at least or up to about 5,000-fold, or at least or up to about 10,000-fold. A gene that modulates protein secretion can decrease protein secretion by at most or less than about 10,000-fold, at most or less than about 5,000-fold, at most or less than about 1,000-fold, at most or less than about 500-fold, at most or less than about 100-fold, at most or less than about 90-fold, at most or less than about 80-fold, at most or less than about 70-fold, at most or less than about 60-fold, at most or less than about 50-fold, at most or less than about 40-fold, at most or less than about 30-fold, at most or less than about 20-fold, at most or less than about 10-fold, at most or less than about 9-fold, at most or less than about 8-fold, at most or less than about 7-fold, at most or less than about 6-fold, at most or less than about 5-fold, at most or less than about 4-fold, at most or less than about 3-fold, at most or less than about 2-fold, or at most or less than about 1-fold.

An expression cassette can comprise at least one regulatory sequence. A regulatory sequence can comprise a promoter, a repressor, an enhancer, a terminator, a 5′ UTR, a 3′ UTR or a combination thereof. A regulatory sequence can be part of a coding sequence. Alternatively, a regulatory sequence can be a non-coding sequence. An expression cassette can comprise at least 1, at least 2, at least 3, at least 4, at least 5, at least 6, at least 7, at least 8, at least 9, at least 10 or more regulatory sequences.

A promoter can be an endogenous P. pastoris promoter or variant thereof. Alternatively, a promoter can be a yeast promoter from a different species (e.g., S. cerevisiae) or variant thereof. Alternatively, a promoter can be a synthetic or novel promoter. A promoter can be an AOX1 promoter, an ADH3 promoter, a TEF1 promoter, a PGK1 promoter, a TPI1 promoter, a PHO89 promoter, a THI11 promoter, an AOD promoter, or a GAP promoter.

An enhancer can be an endogenous P. pastoris enhancer or variant thereof. Alternatively, an enhancer can be a yeast enhancer from a different species (e.g., S. cerevisiae) or variant thereof. Alternatively, an enhancer can be a synthetic or novel enhancer. An enhancer can be an AOX1 enhancer or a GAP enhancer.

A repressor can be an endogenous P. pastoris repressor or variant thereof. Alternatively, a repressor can be a yeast repressor from a different species (e.g., S. cerevisiae) or variant thereof. Alternatively, a repressor can be a synthetic or novel repressor. A repressor can be an AOX1 repressor or a GAP repressor.

A terminator can be an endogenous P. pastoris terminator or variant thereof. Alternatively, a terminator can be a yeast terminator from a different species (e.g., S. cerevisiae) or variant thereof. Alternatively, a terminator can be a synthetic or novel terminator. A terminator can be an AOX1 terminator, a RPL15A terminator, a RPL41B terminator, a DAS1 terminator, a VMA2 terminator, a SRP54 terminator, a FUR4 terminator, a PLB3 terminator, a GIC1 terminator, a FBP1 terminator, a CYC1 terminator, a IDP1 terminator, a PRC1 terminator, a RPL3 terminator, a PGK1 terminator, a YPT31 terminator, an ASP3-1 terminator, a TPO4 terminator, a RPS28A terminator, a VMA16 terminator, a LSC2 terminator, a DIT1 terminator, a MIG2 terminator, an ADH2 terminator, an ADH3 terminator, an ICY2 terminator, a PHO89 terminator, a THI11 terminator, an AOD terminator, or a GAP terminator.

A UTR can be a 5′ UTR or a 3′ UTR. A UTR can be an endogenous P. pastoris UTR or variant thereof. Alternatively, a UTR can be a yeast UTR from a different species (e.g., S. cerevisiae) or variant thereof. Alternatively, a UTR can be a synthetic or novel UTR. A UTR can be an AOX1 UTR, a RPL15A UTR, a RPL41B UTR, a DAS1 UTR, a VMA2 UTR, a SRP54 UTR, a FUR4 UTR, a PLB3 UTR, a GIC1 UTR, a FBP1 UTR, a CYC1 UTR, an IDP1 UTR, a PRC1 UTR, a RPL3 UTR, a PGK1 UTR, a YPT31 UTR, an ASP3-1 UTR, a TPO4 UTR, a RPS28A UTR, a VMA16 UTR, a LSC2 UTR, a DIT1 UTR, a MIG2 UTR, an ADH2 UTR, an ADH3 UTR, a TEF1 UTR, a PGK1 UTR, a TPI1 UTR, a PHO89 UTR, a THI11 UTR, an AOD UTR, or a GAP UTR.

An expression cassette can comprise a signal sequence. A signal sequence (e.g., a secretion sequence or a nuclear localization sequence) can aid with and/or induce protein secretion. An expression cassette can comprise at least 1, at least 2, at least 3, at least 4, at least 5, at least 6, at least 7, at least 8, at least 9, at least 10 or more signal sequences.

A signal sequence can be an endogenous P. pastoris signal sequence or variant thereof. Alternatively, a signal sequence can be a yeast signal sequence from a different species (e.g., S. cerevisiae) or variant thereof. Alternatively, a signal sequence can be a synthetic or novel promoter. A signal sequence can be an amylase signal sequence, an inulinase signal sequence, an invertase signal sequence, a lysozyme signal sequence, an albumin signal sequence, a glucoamylase signal sequence, or a killer protein signal sequence, a S. cerevisiae Ost1 signal sequence, a S. cerevisiae alpha mating factor, a partial S. cerevisiae alpha mating factor, or a Pichia Exg1p signal sequence.

A regulatory sequence can modulate protein yield. A regulatory sequence can enhance protein yield, decrease protein yield, or maintain protein yield.

A regulatory sequence that modulates protein yield can increase protein yield by at least about 5%, at least about 10%, at least about 20%, at least about 30%, at least about 40%, at least about 50%, at least about 60%, at least about 70%, at least about 80%, at least about 90%, at least about 100%, at least about 150%, at least about 200%, at least about 300%, at least about 400%, at least about 500%, at least about 600%, at least about 700%, at least about 800%, at least about 900% at least about 1,000%, at least about 5,000%, at least about 10,000% or more. A regulatory sequence that modulates protein yield can increase protein yield by at most about 10,000%, at most about 5,000%, at most about 1,000%, at most about 900%, at most about 800%, at most about 700%, at most about 600%, at most about 500%, at most about 400%, at most about 300%, at most about 200%, at most about 150%, at most about 100%, at most about 90%, at most about 80%, at most about 70%, at most about 60%, at most about 50%, at most about 40%, at most about 30%, at most about 20%, at most about 10%, at most about 5%, or less.

A regulatory sequence that modulates protein yield can increase protein yield by at least or up to about 1-fold, at least or up to about 2-fold, at least or up to about 3-fold, at least or up to about 4-fold, at least or up to about 5-fold, at least or up to about 6-fold, at least or up to about 7-fold, at least or up to about 8-fold, at least or up to about 9-fold, at least or up to about 10-fold, at least or up to about 20-fold, at least or up to about 30-fold, at least or up to about 40-fold, at least or up to about 50-fold, at least or up to about 60-fold, at least or up to about 70-fold, at least or up to about 80-fold, at least or up to about 90-fold, at least or up to about 100-fold, at least or up to about 500-fold, at least or up to about 1,000-fold, at least or up to about 5,000-fold, or at least or up to about 10,000-fold. A regulatory sequence that modulates protein yield can increase protein yield by at most or less than about 10,000-fold, at most or less than about 5,000-fold, at most or less than about 1,000-fold, at most or less than about 500-fold, at most or less than about 100-fold, at most or less than about 90-fold, at most or less than about 80-fold, at most or less than about 70-fold, at most or less than about 60-fold, at most or less than about 50-fold, at most or less than about 40-fold, at most or less than about 30-fold, at most or less than about 20-fold, at most or less than about 10-fold, at most or less than about 9-fold, at most or less than about 8-fold, at most or less than about 7-fold, at most or less than about 6-fold, at most or less than about 5-fold, at most or less than about 4-fold, at most or less than about 3-fold, at most or less than about 2-fold, or at most or less than about 1-fold.

A regulatory sequence that modulates protein yield can decrease protein yield by at least about 5%, at least about 10%, at least about 20%, at least about 30%, at least about 40%, at least about 50%, at least about 60%, at least about 70%, at least about 80%, at least about 90%, at least about 100%, at least about 150%, at least about 200%, at least about 300%, at least about 400%, at least about 500%, at least about 600%, at least about 700%, at least about 800%, at least about 900% at least about 1,000%, at least about 5,000%, at least about 10,000% or more. A regulatory sequence that modulates protein yield can decrease protein yield by at most about 10,000%, at most about 5,000%, at most about 1,000%, at most about 900%, at most about 800%, at most about 700%, at most about 600%, at most about 500%, at most about 400%, at most about 300%, at most about 200%, at most about 150%, at most about 100%, at most about 90%, at most about 80%, at most about 70%, at most about 60%, at most about 50%, at most about 40%, at most about 30%, at most about 20%, at most about 10%, at most about 5%, or less.

A regulatory sequence that modulates protein yield can decrease protein yield by at least or up to about 1-fold, at least or up to about 2-fold, at least or up to about 3-fold, at least or up to about 4-fold, at least or up to about 5-fold, at least or up to about 6-fold, at least or up to about 7-fold, at least or up to about 8-fold, at least or up to about 9-fold, at least or up to about 10-fold, at least or up to about 20-fold, at least or up to about 30-fold, at least or up to about 40-fold, at least or up to about 50-fold, at least or up to about 60-fold, at least or up to about 70-fold, at least or up to about 80-fold, at least or up to about 90-fold, at least or up to about 100-fold, at least or up to about 500-fold, at least or up to about 1,000-fold, at least or up to about 5,000-fold, or at least or up to about 10,000-fold. A regulatory sequence that modulates protein yield can decrease protein yield by at most or less than about 10,000-fold, at most or less than about 5,000-fold, at most or less than about 1,000-fold, at most or less than about 500-fold, at most or less than about 100-fold, at most or less than about 90-fold, at most or less than about 80-fold, at most or less than about 70-fold, at most or less than about 60-fold, at most or less than about 50-fold, at most or less than about 40-fold, at most or less than about 30-fold, at most or less than about 20-fold, at most or less than about 10-fold, at most or less than about 9-fold, at most or less than about 8-fold, at most or less than about 7-fold, at most or less than about 6-fold, at most or less than about 5-fold, at most or less than about 4-fold, at most or less than about 3-fold, at most or less than about 2-fold, or at most or less than about 1-fold.

A regulatory sequence can modulate protein secretion. A regulatory sequence that modulates protein secretion can enhance protein secretion, decrease protein secretion, stop protein secretion, or maintain protein secretion.

A regulatory sequence that modulates protein secretion can increase protein secretion by at least about 5%, at least about 10%, at least about 20%, at least about 30%, at least about 40%, at least about 50%, at least about 60%, at least about 70%, at least about 80%, at least about 90%, at least about 100%, at least about 150%, at least about 200%, at least about 300%, at least about 400%, at least about 500%, at least about 600%, at least about 700%, at least about 800%, at least about 900% at least about 1,000%, at least about 5,000%, at least about 10,000% or more. A regulatory sequence that modulates protein secretion can increase protein secretion by at most about 10,000%, at most about 5,000%, at most about 1,000%, at most about 900%, at most about 800%, at most about 700%, at most about 600%, at most about 500%, at most about 400%, at most about 300%, at most about 200%, at most about 150%, at most about 100%, at most about 90%, at most about 80%, at most about 70%, at most about 60%, at most about 50%, at most about 40%, at most about 30%, at most about 20%, at most about 10%, at most about 5%, or less.

A regulatory sequence that modulates protein secretion can increase protein secretion by at least or up to about 1-fold, at least or up to about 2-fold, at least or up to about 3-fold, at least or up to about 4-fold, at least or up to about 5-fold, at least or up to about 6-fold, at least or up to about 7-fold, at least or up to about 8-fold, at least or up to about 9-fold, at least or up to about 10-fold, at least or up to about 20-fold, at least or up to about 30-fold, at least or up to about 40-fold, at least or up to about 50-fold, at least or up to about 60-fold, at least or up to about 70-fold, at least or up to about 80-fold, at least or up to about 90-fold, at least or up to about 100-fold, at least or up to about 500-fold, at least or up to about 1,000-fold, at least or up to about 5,000-fold, or at least or up to about 10,000-fold. A regulatory sequence that modulates protein secretion can increase protein secretion by at most or less than about 10,000-fold, at most or less than about 5,000-fold, at most or less than about 1,000-fold, at most or less than about 500-fold, at most or less than about 100-fold, at most or less than about 90-fold, at most or less than about 80-fold, at most or less than about 70-fold, at most or less than about 60-fold, at most or less than about 50-fold, at most or less than about 40-fold, at most or less than about 30-fold, at most or less than about 20-fold, at most or less than about 10-fold, at most or less than about 9-fold, at most or less than about 8-fold, at most or less than about 7-fold, at most or less than about 6-fold, at most or less than about 5-fold, at most or less than about 4-fold, at most or less than about 3-fold, at most or less than about 2-fold, or at most or less than about 1-fold.

A regulatory sequence that modulates protein secretion can decrease protein secretion by at least about 5%, at least about 10%, at least about 20%, at least about 30%, at least about 40%, at least about 50%, at least about 60%, at least about 70%, at least about 80%, at least about 90%, at least about 100%, at least about 150%, at least about 200%, at least about 300%, at least about 400%, at least about 500%, at least about 600%, at least about 700%, at least about 800%, at least about 900% at least about 1,000%, at least about 5,000%, at least about 10,000% or more. A regulatory sequence that modulates protein secretion can decrease protein secretion by at most about 10,000%, at most about 5,000%, at most about 1,000%, at most about 900%, at most about 800%, at most about 700%, at most about 600%, at most about 500%, at most about 400%, at most about 300%, at most about 200%, at most about 150%, at most about 100%, at most about 90%, at most about 80%, at most about 70%, at most about 60%, at most about 50%, at most about 40%, at most about 30%, at most about 20%, at most about 10%, at most about 5%, or less.

A regulatory sequence that modulates protein secretion can decrease protein secretion by at least or up to about 1-fold, at least or up to about 2-fold, at least or up to about 3-fold, at least or up to about 4-fold, at least or up to about 5-fold, at least or up to about 6-fold, at least or up to about 7-fold, at least or up to about 8-fold, at least or up to about 9-fold, at least or up to about 10-fold, at least or up to about 20-fold, at least or up to about 30-fold, at least or up to about 40-fold, at least or up to about 50-fold, at least or up to about 60-fold, at least or up to about 70-fold, at least or up to about 80-fold, at least or up to about 90-fold, at least or up to about 100-fold, at least or up to about 500-fold, at least or up to about 1,000-fold, at least or up to about 5,000-fold, or at least or up to about 10,000-fold. A regulatory sequence that modulates protein secretion can decrease protein secretion by at most or less than about 10,000-fold, at most or less than about 5,000-fold, at most or less than about 1,000-fold, at most or less than about 500-fold, at most or less than about 100-fold, at most or less than about 90-fold, at most or less than about 80-fold, at most or less than about 70-fold, at most or less than about 60-fold, at most or less than about 50-fold, at most or less than about 40-fold, at most or less than about 30-fold, at most or less than about 20-fold, at most or less than about 10-fold, at most or less than about 9-fold, at most or less than about 8-fold, at most or less than about 7-fold, at most or less than about 6-fold, at most or less than about 5-fold, at most or less than about 4-fold, at most or less than about 3-fold, at most or less than about 2-fold, or at most or less than about 1-fold.

A food product or substance can be comprised of the recombinant proteins described herein. The food product or substance may comprise recombinant proteins described herein provided in an isolated or purified form. Alternatively, or in addition to, the recombinant proteins described herein may be provided to the food product or substance as whole cells comprising the recombinant proteins. A food product or substance can be a food for animals. A food product or substance can be food for a pet (e.g., companion animal), a farm or agricultural animal, a wild animal, or a human. A food product or substance can be food for a obligate carnivore (e.g., a feline), which requires animal protein as a part of the diet.

A food product can be a wet puree. Alternatively, a food product can be a dry pellet. A food product can replace an animal or human diet. Alternatively, a food product can supplement an animal or human diet.

A food product can contain additional ingredients such as, but not limited to, animal-based meat, amino acid supplements (e.g., taurine), vitamins (e.g., vitamin E), fats, nutritional yeast, spirulina, agar, xanthan gum, kelp powder, squash powder, sweet potato powder, papaya powder, flaxseed oil, coconut oil, probiotics, cranberry powder, wheat grass powder, turmeric, coconut flour, chitosan, pumpkin puree, inulin powder, guar gum, green tea extract, koji, stabilizers, vegetables, fruits, microbes, fillers, or flavoring compounds.

### EXAMPLES

### Example 1: Recombinant Protein Expression

In this experiment, a Pichia pastoris cell was transfected with a DNA sequence, resulting in recombinant protein expression.

Host cells were chosen based on predetermined characteristics such as, but not limited to, doubling time, ability to properly fold and make appropriate post-translational modifications on recombinant proteins, protein secretion, ability to grow on cost-effective and/or easily available media/feedstock, or genetic tractability. Host cells that do not efficiently secrete proteins may be lysed to release the target recombinant proteins.

Pichia cells were grown overnight in YPD media in baffled flasks at 30° C. to an optical density at 600 nm wavelength (OD 600) of about 1 to 2.6. Cells were collected by centrifugation and resuspended at room temperature for 30 min in 100 mM lithium acetate (LiOAc), 10 mM dithiothreitol (DTT), 0.6M sorbitol, and 10 mM Tris-HCl pH 7.5, at 1e8 cells/ml. Cells were pelleted and washed four times in 1 mL 1M sorbitol. Cells were resuspended in 1M sorbitol at a final concentration of 1e9 cells/ml. 40 μL of cell suspension was mixed with 1 uL (100-1 ug) of DNA, transferred to a 0.2 cm gap cuvette, and incubated on ice for 5 minutes.

The expression cassette used comprised a promoter, a 5′ untranslated region, a signal sequence for protein secretion, an open reading frame (ORF) of an animal muscle protein, a 3′ UTR, and a transcriptional termination and 3′ end processing sequence. The construct used a P. pastoris AOX1 promoter, a P. pastoris 5′ UTR, a P. pastoris 3′ UTR, a S. cerevisiae alpha mating factor, a rabbit serum albumin ORF, and a P. pastoris terminator.

The expression cassette was transfected into the P. pastoris host cell using electroporation at 1.5 kV, 200 ohms, and 25 uF, where the expression cassette was integrated into the genome of P. pastoris. Electroporated cells were diluted in 1 mL 1M sorbitol. Cells were transferred to 15 ml round-bottom tubes with 1 ml YPD media and incubated while shaking at 30° C. for 1 hour.

Cells were plated on YPDS plates and incubated at 30° C. Once the cells' endogenous protein expression machinery expressed the protein of interest, the cells secreted the protein of interest into the media, which was harvested and purified for further use.

### Example 2: Analysis of Secreted Proteins

In this experiment, genes of interest were cloned into plasmids containing the GAP1 promotor, Saccharomyces cerevisiae alpha-Mating factor signal sequence, and a zeocin resistance gene. Plasmids were linearized and transformed into Pichia pastoris cells using electroporation. Transformed cells were spread onto YPDS plates containing 100 micrograms per milliliter (ug/mL) zeocin and incubated at 30° C. for 3-4 days. Colonies were restruck to single colony twice. To produce secreted protein, cells were resuspended in 4 milliliters (mL) of buffered complex glycerol medium (BMGY) (rich glycerol) and cultured shaking at 250 revolutions per minute (rpm) and 30° C. for 4 days. Every 24 hours, 200 microliters (uL) of culture was removed and centrifuged to separate cells from supernatant. A 10 μL aliquot of supernatant was used to analyze secreted proteins on a sodium dodecyl sulfate-polyacrylamide gel electrophoresis (SDS-PAGE) gel.

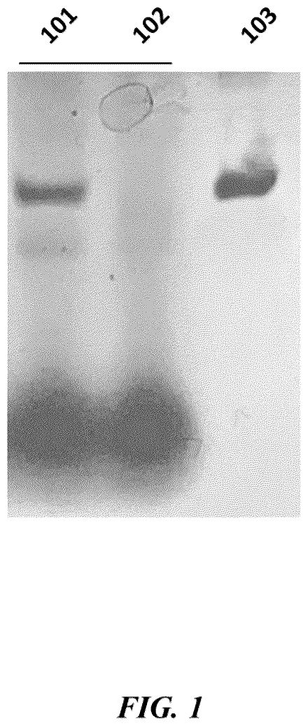

FIG. 1 shows a Coomassie stained SDS-PAGE gel of an example media from Pichia pastoris cells including a construct for secreted recombinant rabbit albumin or wildtype cells. Wildtype cells lack the construct for secreted recombinant rabbit albumin. Lane 1 101 shows a band at 66 kilo Daltons (kDa), which corresponds to the recombinant rabbit albumin. Lane 2 102, which correspond to the wildtype media, lacks a band at 66 kDa. Lane 3 103 shows a bovine serum albumin marker with a band at 66 kDa.

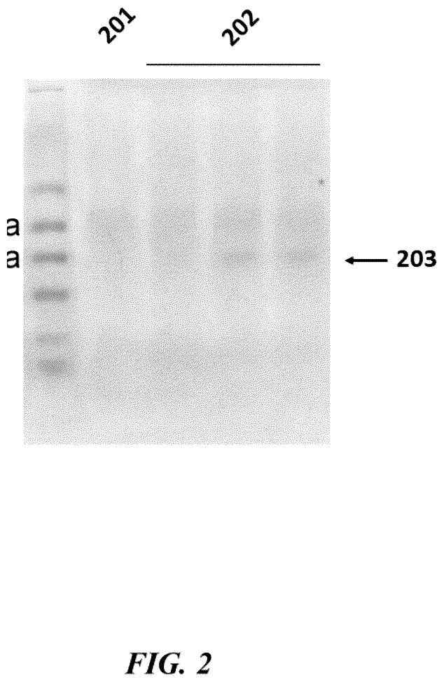

FIG. 2 shows a Coomassie stained sodium SDS-PAGE gel of an example media from Pichia pastoris cells including a construct for secreted recombinant rabbit Tropomyosin alpha-1 chain or wildtype cells. The wildtype cells lack the construct. Lane 2 201 shows the wildtype expressed protein from wildtype cells. Lanes 3-5 202 show secreted protein expression for Tropomyosin alpha-1 chain. Lanes 3-5 show a band 203 at 33 kDa corresponding to the Tropomyosin alpha-1 chain.

For proteins produced in a bioreactor, cells were resuspended in YPG medium and grown in shake flasks at 30° C. for 16 hours. The cells were transferred to seed fermenters and grown at 30° C. for another 16 hours. The cultures were transferred to a 100 liter (L) fermenter and batch fed a glycerol-based media for 92 hours at 30° C. The culture was then centrifuged to separate solid and liquid phases, filtered, and spray dried to produce the final protein powder.

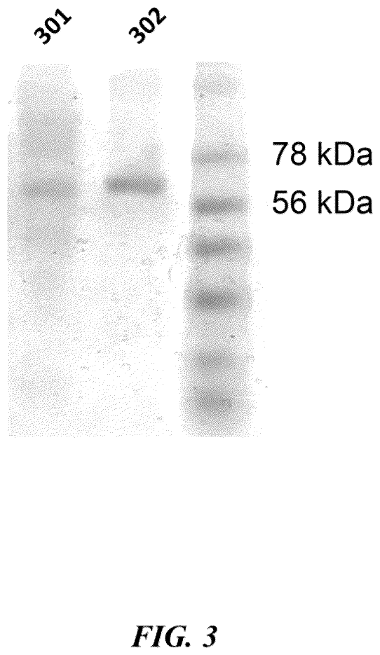

FIG. 3 shows a Coomassie stained SDS-PAGE gel of an example recombinant rabbit albumin. Lane 1 301 shows an example recombinant rabbit albumin (rALBU-RABIT) grown from a bioreactor. The rabbit albumin peptides were purified and spray dried. The protein powder was resuspended and run on an SDS-PAGE gel. Lane 2 302 shows a bovine serum albumin marker.

### Example 3: Cat Food with Rabbit Proteins

In this example, cat food is made using rabbit proteins. Rabbits are naturally hunted by felines in the wild, and rabbit proteins are hypoallergenic for cats. A recombinant rabbit protein is produced and used as an ingredient in both wet and dry cat food, snacks, and/or treats. In some formulations, the protein is in dried powder form. In some formulations, the protein is in a solution. For dry food, ingredients are mixed and extruded.

Twenty-five rabbit proteins are produced using the recombinant protein expression method of Example 1. The twenty-five rabbit proteins tested are found in Table 2.

The rabbit proteins are harvested from the recombinant protein expression method and produced into a wet pureed cat treat. Additionally, the rabbit proteins are harvested from the recombinant protein expression method and produced into a dry extruded cat food kibble.

### Example 4: Pet Food with Cow Proteins

In this example, pet food is made using cow proteins. A recombinant cow protein is produced and used as an ingredient in both wet and dry pet food, snacks, and/or treats. In some formulations, the protein is in dried powder form. In some formulations, the protein is in a solution. For dry food, ingredients are mixed and extruded.

The cow proteins are produced using the recombinant protein expression method of Example 1. The cow proteins tested are found in Table 3.

### Example 5: Pet Food with Sheep Proteins

In this example, pet food is made using sheep proteins. A recombinant sheep protein is produced and used as an ingredient in both wet and dry pet food, snacks, and/or treats. In some formulations, the protein is in dried powder form. In some formulations, the protein is in a solution. For dry food, ingredients are mixed and extruded.

The sheep proteins are produced using the recombinant protein expression method of Example 1. The sheep proteins tested are found in Table 4.

### Example 6: Pet Food with Pig Proteins

In this example, pet food is made using pig proteins. A recombinant pig protein is produced and used as an ingredient in both wet and dry pet food, snacks, and/or treats. In some formulations, the protein is in dried powder form. In some formulations, the protein is in a solution. For dry food, ingredients are mixed and extruded.

The pig proteins are produced using the recombinant protein expression method of Example 1. The pig proteins tested are found in Table 5.

### Example 7: Pet Food with Bison Proteins

In this example, pet food is made using bison proteins. A recombinant bison protein is produced and used as an ingredient in both wet and dry cat food, snacks, and/or treats. In some formulations, the protein is in dried powder form. In some formulations, the protein is in a solution. For dry food, ingredients are mixed and extruded.

The bison proteins are produced using the recombinant protein expression method of Example 1. The bison proteins tested are found in Table 6.

### Example 8: Pet Food with Deer Proteins

In this example, pet food is made using deer proteins. A recombinant deer protein is produced and used as an ingredient in both wet and dry pet food, snacks, and/or treats. In some formulations, the protein is in dried powder form. In some formulations, the protein is in a solution. For dry food, ingredients are mixed and extruded.

The deer proteins are produced using the recombinant protein expression method of Example 1. The deer proteins tested are found in Table 7.

### Example 9: Pet Food with Chicken Proteins

In this example, pet food is made using chicken proteins. A recombinant chicken protein is produced and used as an ingredient in both wet and dry pet food, snacks, and/or treats. In some formulations, the protein is in dried powder form. In some formulations, the protein is in a solution. For dry food, ingredients are mixed and extruded.

The chicken proteins are produced using the recombinant protein expression method of Example 1. The chicken proteins tested is found in Table 8.

### Example 10: Pet Food with Turkey Proteins

In this example, cat food is made using turkey proteins. A recombinant turkey protein is produced and used as an ingredient in both wet and dry pet food, snacks, and/or treats. In some formulations, the protein is in dried powder form. In some formulations, the protein is in a solution. For dry food, ingredients are mixed and extruded.

The turkey proteins are produced using the recombinant protein expression method of Example 1. The turkey proteins tested are found in Table 9.

### Example 11: Cat Food with Duck Proteins

In this example, cat food is made using duck proteins. Ducks are naturally hunted by felines in the wild, and duck proteins are hypoallergenic for cats. A recombinant duck protein is produced and used as an ingredient in both wet and dry cat food, snacks, and/or treats. In some formulations, the protein is in dried powder form. In some formulations, the protein is in a solution. For dry food, ingredients are mixed and extruded.

The duck proteins are produced using the recombinant protein expression method of Example 1. The duck proteins tested are found in Table 10 and Table 11.

### Example 12: Pet Food with Salmon Proteins

In this example, pet food is made using salmon proteins. A recombinant salmon protein is produced and used as an ingredient in both wet and dry pet food, snacks, and/or treats. In some formulations, the protein is in dried powder form. In some formulations, the protein is in a solution. For dry food, ingredients are mixed and extruded.

The salmon proteins are produced using the recombinant protein expression method of Example 1. The salmon proteins tested are found in Table 12.

### Example 13: Pet Food with Cod Proteins

In this example, pet food is made using cod proteins. A recombinant cod protein is produced and used as an ingredient in both wet and dry pet food, snacks, and/or treats. In some formulations, the protein is in dried powder form. In some formulations, the protein is in a solution. For dry food, ingredients are mixed and extruded.

The cod proteins are produced using the recombinant protein expression method of Example 1. The cod proteins tested are found in Table 13.

### Example 14: Pet Food with Tuna Proteins

In this example, pet food is made using tuna proteins. A recombinant tuna protein is produced and used as an ingredient in both wet and dry pet food, snacks, and/or treats. In some formulations, the protein is in dried powder form. In some formulations, the protein is in a solution. For dry food, ingredients are mixed and extruded.

The tuna proteins are produced using the recombinant protein expression method of Example 1. The tuna protein tested is found in Table 14.

### Example 15: Food Product for Human Consumption

In this example, a food product for human consumption is made using any of the proteins described in Tables 2-14. A recombinant protein from Tables 2-14 is produced and provided in a food, beverage, or supplement. In some formulations, the protein is in dried powder form. In some formulations, the protein is in a solution.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

1.-41. (canceled)

42. A method for producing a pet food product, the method comprising:
(a) contacting a plurality of host cells with a recombinant nucleic acid, wherein the recombinant nucleic acid encodes an albumin protein of an animal selected from the group consisting of a mammal, a bird, and a fish;
(b) expressing the albumin protein encoded by the recombinant nucleic acid in the plurality of host cells; and
(c) generating a pet food product comprising the albumin protein expressed in the plurality of host cells.

43. The method of claim 42, wherein the albumin protein is a rabbit albumin protein.

44. The method of claim 42, wherein host cells of the plurality of host cells comprise one or more members selected from the group consisting of Pichia cells, Saccharomyces cells, Schizosaccharomyces cells, Trichoderma cells, Aspergillus cells, Escherichia cells, Bacillus cells, and Lactococcus cells.

45. The method of claim 42, wherein the albumin protein comprises an amino acid sequence that is at least 70% identical to any one of SEQ ID NOs: 16, 78, 93, 108, 123, 138, 161, 174, 187, 200, 215, or 216.

46. The method of claim 45, wherein the amino acid sequence is at least 70% identical to SEQ ID NO 78.

47. The method of claim 45, wherein the amino acid sequence is at least 90% identical to any one of SEQ ID NOs: 16, 78, 123, 161, 174, 187, 200, 215, or 216.

48. The method of claim 45, wherein the amino acid sequence is at least 95% identical to any one of SEQ ID NOs: 16, 78, 108, 123, 161, 174, 187, 200, 215, or 216.

49. The method of claim 42, further comprising isolating the albumin protein from the plurality of host cells.

50. A method for producing a pet food product, the method comprising:
(a) contacting a plurality of host cells with a first recombinant nucleic acid encoding an albumin protein and a second recombinant nucleic acid that encodes a protein different from the albumin protein;
(b) expressing the albumin protein encoded by the first recombinant nucleic acid and the protein encoded by the second recombinant nucleic acid in the plurality of host cells; and
(c) generating a pet food product comprising the albumin protein and the protein expressed in the plurality of host cells.

51. The method of claim 50, further comprising isolating the albumin protein and the protein from the plurality of host cells.

52. The method of claim 50, wherein the albumin protein or the protein is a rabbit protein.

53. The method of claim 52, wherein the albumin protein and the protein are rabbit proteins.

54. The method of claim 50, wherein the albumin protein comprises an amino acid sequence that is at least 70% identical to any one of SEQ ID NOs: 16, 78, 93, 108, 123, 138, 161, 174, 187, 200, 215, or 216.

55. The method of claim 54, wherein the amino acid sequence is at least 70% identical to SEQ ID NO 78.

56. The method of claim 50, wherein the protein comprises an amino acid sequence that is at least 70% identical to any one of SEQ ID NOs: 1-15, 17-77, 79-92, 94-107, 109-122, 124-137, 139-160, 162-173, 175-186, 188-199, 201-204, or 217-232.

57. The method of claim 56, wherein the amino acid sequence is at least 70% identical to any one of SEQ ID NOs: 4, 54, 57, 58, 59, 60, 63, 64, 67, 74, or 77.

58. A method for producing a pet food product, the method comprising:
(a) contacting a plurality of host cells with a recombinant nucleic acid;
(b) expressing a protein encoded by the recombinant nucleic acid; and
(c) generating a pet food product comprising the protein expressed in the plurality of host cells,
wherein the protein comprises an amino acid sequence that is at least 70% identical to any one of SEQ ID NOs: 5, 8, 9, 11, 14, 16, 19-21, 23, 24, 26-29, 32-35, 37-41, 44-49, 51-54, 56, 57, 59, 65-79, 81, 82, 84, 93, 94, 96, 97, 99, 108, 109, 111, 112, 114, 117, 123, 124, 126, 127, 129, 138-141, 143, 147, 148, 150, 151, 153, 161, 162, 164, 165, 167, 169, 170, 172, 174, 175, 177, 178, 180, 184, 185, 187, 188, 190-192, 197, 200, 201, 203-206, 208, 209, 213, 215, 216, 217, 219-222, 224, 225, or 229.

59. The method of claim 58, wherein the protein is a rabbit protein.

60. The method of claim 58, wherein the protein is an albumin protein.

61. The method of claim 58, wherein the amino acid sequence is any one of SEQ ID NOs: 5, 8, 9, 11, 14, 16, 19-21, 23, 24, 26-29, 32-35, 37-41, 44-49, 51-54, 56, 57, 59, 65-79, 81, 82, 84, 93, 94, 96, 97, 99, 108, 109, 111, 112, 114, 117, 123, 124, 126, 127, 129, 138-141, 143, 147, 148, 150, 151, 153, 161, 162, 164, 165, 167, 169, 170, 172, 174, 175, 177, 178, 180, 184, 185, 187, 188, 190-192, 197, 200, 201, 203-206, 208, 209, 213, 215, 216, 217, 219-222, 224, 225, or 229.
